(19) United States
(12) Patent Application Publication
(10) Pub. No.: US 2023/0295183 A1
(43) Pub. Date: Sep. 21, 2023

(54) HOT MELT EXTRUDED SOLID DISPERSIONS CONTAINING A BCL2 INHIBITOR

(71) Applicant: Newave Pharmaceutical Inc., Pleasanton, CA (US)

(72) Inventor: Yi Chen, Pleasanton, CA (US)

(21) Appl. No.: 17/798,904

(22) PCT Filed: Feb. 23, 2021
(86) PCT No.: PCT/US2021/019181

§ 371 Date: Aug. 11, 2022

Related U.S. Application Data
(60) Provisional application No. 63/053,565, filed on Jul. 17, 2020.
(60) Provisional application No. 62/980,700, filed on Feb. 24, 2020.

(51) Int. Cl. C07D 498/14; A61K 9/20

(52) U.S. Cl. CPC C07D 498/14; A61K 9/2095; A61K 9/2027; A61K 9/2013; A61K 9/2009

(57) ABSTRACT

A pro-apoptotic solid dispersion comprises, a Bcl-2 family protein inhibitory compound of Formula A as defined herein, dispersed in a solid matrix that comprises (a) a pharmaceutically acceptable water-soluble polymeric carrier, and (b) a pharmaceutically acceptable surfactant. A process for preparing such a solid dispersion comprises subjecting to elevated temperature the compound of Formula A, the water-soluble polymeric carrier, and the surfactant to provide an extrudable semi-solid mixture; extruding the semi-solid mixture; and cooling the resulting extrudate to provide a solid matrix comprising the polymeric carrier, and the surfactant and having the compound dispersed in essentially non-crystalline form therein. The solid dispersion is suitable for oral administration to a subject in need thereof for treatment of a disease characterized by overexpression of one or more anti-apoptotic Bcl-2 family proteins, for example cancer or an immune or autoimmune disease.

### REFERENCE TO RELATED APPLICATIONS

This application claims the benefit of the filing date of U.S. Provisional Patent Application Nos. 62/980,700, filed on Feb. 24, 2020; and 63/053,565, filed on Jul. 17, 2020, the entire contents of each of the above-referenced applications are incorporated herein by reference.

### FIELD OF THE INVENTION

The present invention relates to solid dispersions comprising an apoptosis-inducing agent, to pharmaceutical dosage forms comprising such dispersions, to processes for preparing such dispersions and dosage forms and to methods of use thereof for treating diseases characterized by overexpression of anti-apoptotic Bcl-2 family proteins.

### BACKGROUND OF THE INVENTION

Apoptosis, or programmed cell death, is a conserved and regulated process that is the primary mechanism for the removal of aged, damaged and unnecessary cells. The ability to block apoptotic signaling is a key hallmark of cancer and is thus important for oncogenesis, tumor maintenance and chemoresistance [Hanahan, D. & Weinberg, R. A. The hallmarks of cancer. Cell 100, 57-70 (2000).]. Dynamic binding interactions between prodeath (for example, BCL-2-associated X protein (BAX), BCL-2 antagonist/killer 1 (BAK), BCL-2-associated agonist of cell death (BAD), BCL-2-like 11 (BIM), NOXA and BCL-2 binding component 3 (PUMA)) and prosurvival (BCL-2, BCL-XL, BCL-2-like 2 (BCL-W), myeloid cell leukemia sequence 1 (MCL-1) and BCL-2-related protein A1 (BFL-1)) proteins in the BCL-2 family control commitment to programmed cell death. Altering the balance among these opposing factions provides one means by which cancer cells undermine normal apoptosis and gain a survival advantage [Youle, R. J. & Strasser, A. The BCL-2 protein family: opposing activities that mediate cell death. Nat. Rev. Mol. Cell Biol. 9, 47-59 (2008)].

BCL-2, the first identified apoptotic regulator, was originally cloned from the breakpoint of a t(14;18) translocation present in human B cell lymphomas [Tsujimoto, Y., et al. Science 228, 1440-1443 (1985); Cleary, M. L., et al Cell 47, 19-28 (1986); Boise, L. H. et al. Cell 74, 597-608 (1993)]. This protein has since been shown to have a dominant role in the survival of multiple lymphoid malignancies [Vaux, D. L., et al pre-B cells. Nature 335, 440-442 (1988)]. Overexpression of BCL-2 proteins correlates with resistance to chemotherapy, clinical outcome, disease progression, overall prognosis or a combination thereof in various cancers and disorders of the immune system. Involvement of BCL-2 proteins in bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, and the like is described in PCT/US2004/36770, published as WO 2005/049593, and PCT/US2004/37911, published as WO/2005/049594. Involvement of BCL-2 proteins in immune and autoimmune diseases is described in Current Allergy and Asthma Reports 2003, 3, 378-384; British Journal of Hematology 2000, 110(3), 584-90; Blood 2000, 95(4), 1283-92; and New England Journal of Medicine 2004, 351(14), 1409-1418. Involvement of BCL-2 proteins in arthritis is disclosed in WO 2009/064938. Involvement of BCL-2 proteins in bone marrow transplant rejection is disclosed in US 2008-0182845 A1. All incorporated herein by reference.

In the last decade, several BCL-2 inhibitors such as ABT-737, ABT-263, and ABT-199 as shown below have been identified and entered human clinical trials for cancers treatment.

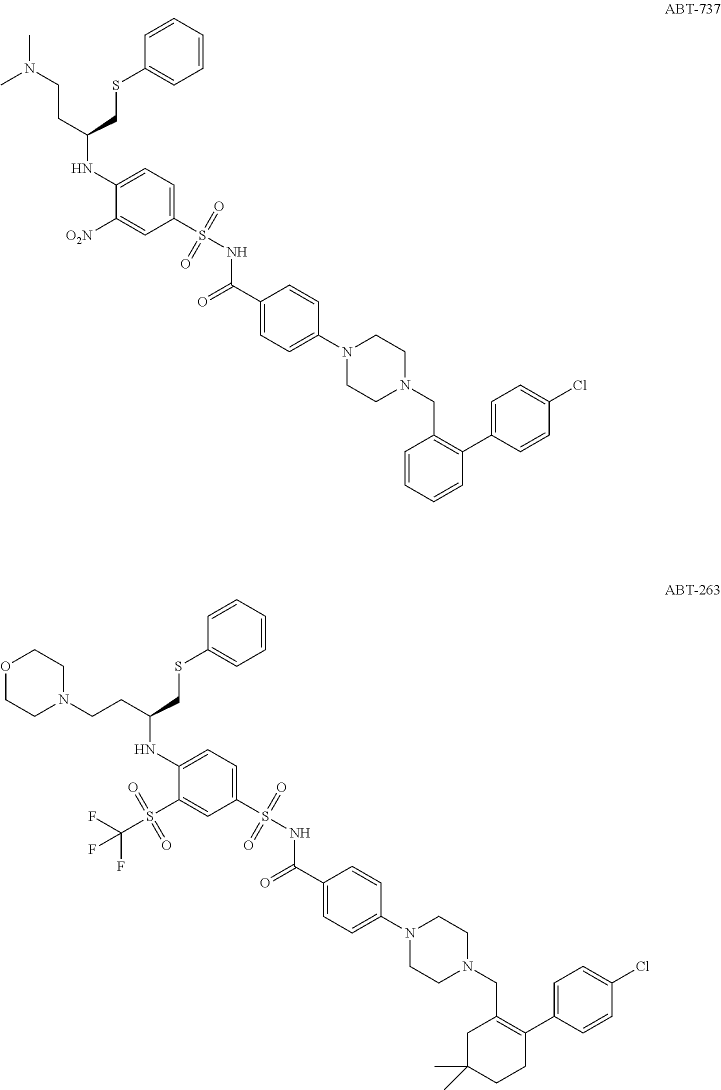 [SMILES: CN(C)CC[C@@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; CC1(C)CCC(c2ccc(Cl)cc2)=C(CN2CCN(c3ccc(C(=O)NS(=O)(=O)c4ccc(N[C@@H](CCN5CCOCC5)CSc5ccccc5)c(S(=O)(=O)C(F)(F)F)c4)cc3)CC2)C1]

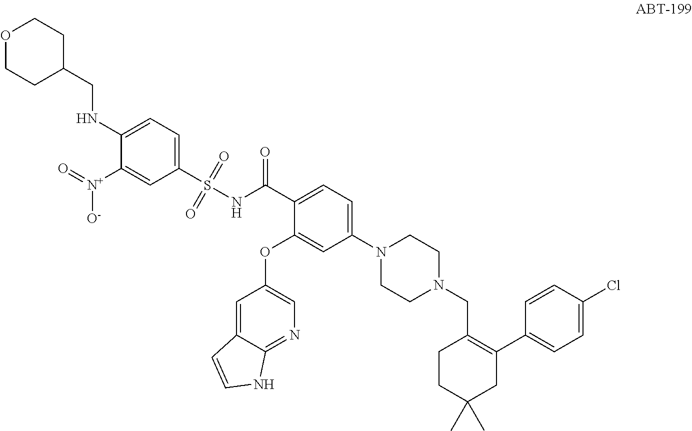 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

ABT-737 is discovered by nuclear magnetic resonance (NMR)-based screening, parallel synthesis and structure based fragment drug design [Tillman Oltersdorf, et al, Nature, Vol 435, 2005, p 677]. ABT-737 a small-molecule inhibitor of the anti-apoptotic proteins BCL-2, Bcl-XL and Bcl-w, with an affinity two to three orders of magnitude more potent than previously reported compounds. Mechanistic studies reveal that ABT-737 does not directly initiate the apoptotic process, but enhances the effects of death signals, displaying synergistic cytotoxicity with chemotherapeutics and radiation. ABT-737 exhibits single-agent-mechanism-based killing of cells from lymphoma and small-cell lung carcinoma lines, as well as primary patient-derived cells, and in animal models, ABT-737 improves survival, causes regression of established tumors, and produces cures in a high percentage of the mice. Unfortunately, ABT-737 is not orally bioavailable, and its formulation for intravenous delivery is hampered by its low aqueous solubility.

After extensive MedChem effort, an orally bioavailable BCL-2 inhibitor ABT-263 (Navitoclax) has been developed [Cheol-Min Park, et al J. Med. Chem. 2008, 51, 6902-6915]. ABT-263 is a potent inhibitor of Bcl-xL, BCL-2 and Bcl-w with Ki of ≤0.5 nM, ≤1 nM and ≤1 nM. ABT-263 has an IC₅₀ of 110 nM against SCLC H146 cell line. When ABT-263 is administered at 100 mg/kg/day in the H345 xenograft model, significant antitumor efficacy is observed with 80% TGI and 20% of treated tumors indicating at least a 50% reduction in tumor volume. Oral administration of ABT-263 alone causes complete tumor regressions in xenograft models of small-cell lung cancer and acute lymphoblastic leukemia [Tse C, et al. Cancer Res. 2008, 68(9), 3421-3428]. In the clinical trial, however, the inhibition of BCL-XL by ABT-263 (navitoclax) induces a rapid, concentration-dependent decrease in the number of circulating platelets. This mechanism-based thrombocytopenia is the dose-limiting toxicity of single-agent navitoclax treatment in patients and limits the ability to drive drug concentrations into a highly efficacious range.

Thus, a BCL-2 selective (BCL-XL sparing) inhibitor would culminate in substantially reduced thrombocytopenia while maintaining efficacy in lymphoid malignancies. The resulting increase in the therapeutic window should allow for greater BCL-2 suppression and clinical efficacy in BCL-2-dependent tumor types. After extensive MedChem, ABT-199 (GDC-0199) has been successfully developed [Andrew J Souers, et al, Nature Medicine, Volume 19, 22, p202, 2013]. ABT-199 is a BCL-2-selective inhibitor with Ki of <0.01 nM, >4800-fold more selective versus Bcl-xL and Bcl-w, and no activity to Mcl-1. ABT-199 potently inhibits RS4;11 cells with EC₅₀ of 8 nM. In addition, ABT-199 induces a rapid apoptosis in RS4;11 cells with cytochrome c release, caspase activation, and the accumulation of sub-G0/G1 DNA. Quantitative immunoblotting reveals that sensitivity to ABT-199 correlated strongly with the expression of BCL-2, including NHL, DLBCL, MCL, AML and ALL cell lines. ABT-199 also induces apoptosis in CLL with an average EC₅₀ of 3.0 nM. A single dose of 100 mg/kg of ABT-199 causes a maximal tumor growth inhibition of 95% and tumor growth delay of 152% in RS4;11 xenografts. ABT-199 also inhibits xenograft growth (DoHH2, Granta-519) as a single agent or in combination with Bendamustine and other agents. Human Phase I and II data showed that ABT-199 is highly efficacious for CLL who have 17p deletion, and was approved by FDA in 2016.

WO/2017/132474, WO/2019/040550, WO/2019/040573, PCT/US2019/047404 and PCT/US2019/047403 disclosed a novel class of BCL-2 inhibitors. Where aqueous solubility of Bcl-2 binding compounds is very low, the formulator faces a significant challenge in assuring acceptable oral bioavailability, which is strongly dependent on solubility in the aqueous medium of the gastrointestinal tract. This is true even where binding affinity is very high. The challenge becomes even greater when considering the need to provide an adequate drug loading in the formulation, so that a therapeutically effective dose can be administered in an acceptably small volume of formulated product.

Liquid dosage forms (including encapsulated liquids) can be useful for some drugs of low aqueous solubility, provided a suitable pharmaceutically acceptable solvent system (generally lipid-based) can be found that provides adequate drug loading without posing solubility or storage-stability issues. Other approaches that have been proposed for such drugs include solid dispersions, which bring their own challenges.

For a variety of reasons, such as patient compliance and taste masking, a solid dosage form is usually preferred over a liquid dosage form. In most instances, however, oral solid dosage forms of a drug provide a lower bioavailability than oral solutions of the drug.

There have been attempts to improve the bioavailability provided by solid dosage forms by forming solid dispersions, or more particularly solid solutions, of drugs. Solid dispersions, or solutions, are preferred physical systems because the components therein readily form liquid solutions when contacted with a liquid medium, such as gastric juice. The ease of dissolution may be attributed at least in part to the fact that the energy required for dissolution of the components from a solid dispersion, or solid solution, is less than that required for the dissolution of the components from a crystalline or microcrystalline solid phase. It is, however, important that the drug released from the solid dispersion, or solid solution, remains water-solubilized in the aqueous fluids of the gastrointestinal tract; otherwise, the drug may precipitate in the gastrointestinal tract, resulting in low bioavailability.

International Patent Publication WO 01/00175 relates to mechanically stable pharmaceutical dosage forms which are solid solutions of active ingredients in an auxiliary agent matrix. The matrix contains a homopolymer or a copolymer of N-vinyl pyrrolidone and a liquid or semi-solid surfactant.

International Patent Publication WO 00/57854 relates to mechanically stable pharmaceutical dosage forms for oral administration, containing at least one active compound, at least one thermoplastically moldable, matrix-forming auxiliary and more than 10% and up to 40% by weight of a surface-active substance that has a hydrophilic-lipophilic balance (HLB) value of between 2 and 18 and is liquid at 20° C., or has a drop point between 20° C. and 50° C.

U.S. Patent Application Publication No. 2005/0208082 relates to a solubilizing composition comprising a mixture of TPGS (a-tocopheryl polyethylene glycol succinate or vitamin E polyethylene glycol succinate) and linoleic acid. The solubilizing composition is used to disperse a lipophile in an aqueous phase.

Hot melt-extrusion, an enabling technology in increasing use for enhancing bioavailability of poorly water-soluble drug compounds, is a solvent-free, non-ambient process that has been said to afford many advantages over conventional solid dosage forms in terms of robustness and versatility (Crowley et al. (2007) Drug Development and Industrial Pharmacy 33:908-926).

Hot melt-extrusion technology has been shown to significantly improve the pharmacokinetic properties of certain drugs over alternative formulations. See, for example, Klein et al. (2007) J. Acquir. Immune Defic. Syndr. 44:401-410.

Apoptosis-inducing drugs that target Bcl-2 family proteins such as Bcl-2 and Bcl-Xx are best administered according to a regimen that provides continual, for example daily, replenishment of the plasma concentration, to maintain the concentration in a therapeutically effective range. This can be achieved by daily parenteral, e.g., intravenous (i.v.) or intraperitoneal (i.p.) administration. However, daily parenteral administration is often not practical in a clinical setting, particularly for outpatients. To enhance clinical utility of an apoptosis-inducing agent, for example as a chemotherapeutic in cancer patients, a solid dosage form with acceptable oral bioavailability would be highly desirable. Such a dosage form, and a regimen for oral administration thereof, would represent an important advance in treatment of many types of cancer, including NHL, CLL and ALL, and would more readily enable combination therapies with other chemotherapeutics.

### SUMMARY OF THE INVENTION

One aspect of the invention described herein provides a solid dispersion (such as hot-melt extrusion) comprising a compound of Formula (A), in essentially non-crystalline (e.g., amorphous) form,

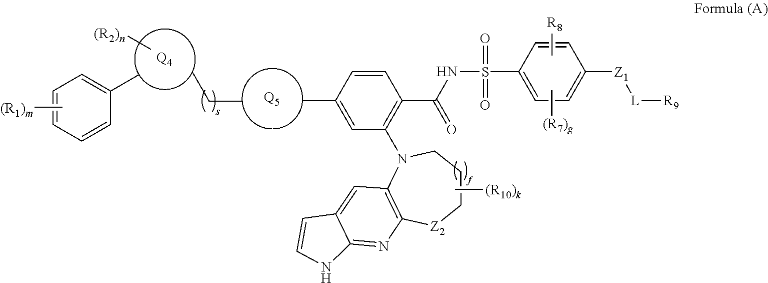

wherein

Q₄ is cycloalkyl, cycloalkenyl, heterocycloalkyl, heterocycloalkenyl, aryl, heteroaryl, or spiro heterocyclic;

Q₅ is cycloalkyl, cycloalkenyl, heterocycloalkyl, heterocycloalkenyl, aryl, heteroaryl, or spiro heterocyclic;

each of R₁, R₂, R₇, R₈, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c},-alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more Rd;

Rₐ, R_{b}, Rₑ, R_{bb}, R_{cc}, and R_{d}, independently, is H, D, alkyl, spiroalkyl, alkenyl, alkynyl, alkoxy, alkoxyalkyl, haloalkyl, hydroxyalkyl, aminoalkyl, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;

Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;

Z₁ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;

L is-L₁-L₂-;

L₁ is a bond, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally substituted with one or more R_{d};

L₂ is a bond, or an alkyl in which one or more -Lᵢ—are optionally inserted between any two adjacent carbon atoms;

-Lᵢ- is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;

two of R₁ group, taken together with the atoms to which they are attached, may optionally form a cycloalkyl or heterocycloalkyl, in which said cycloalkyl or heterocycloalkyl of R₁, is optionally substituted with one or more R_{d};

two of R₂ group, taken together with the atom(s) to which they are attached, may optionally form a cycloalkyl or heterocycloalkyl, in which said cycloalkyl or heterocycloalkyl of R₂, is optionally substituted with one or more R_{d};

two of R₇ group, taken together with the atoms to which they are attached, may optionally form a cycloalkyl or heterocycloalkyl, in which said cycloalkyl or heterocycloalkyl of R₇, is optionally substituted with one or more R_{d};

two of R₁₀ group, taken together with the atom(s) to which they are attached, may optionally form a cycloalkyl or heterocycloalkyl, in which said cycloalkyl or heterocycloalkyl of R₁₀, is optionally substituted with one or more R_{d};

R₇ and L group, taken together with the atom to which they are attached, may optionally form a cycloalkyl, or heterocycloalkyl, in which said cycloalkyl or heterocycloalkyl of R₇ and L, is optionally substituted with one or more Rₑ;

R_{b} and Rₑ group, taken together with the atom to which they are attached, may optionally form a cycloalkyl, or heterocycloalkyl, in which said cycloalkyl or heterocycloalkyl of R_{b} and Rₑ, is optionally substituted with one or more Rₑ;

two of R_{d} group, taken together with the atom(s) to which they are attached, may optionally form a cycloalkyl, or heterocycloalkyl, in which said cycloalkyl or heterocycloalkyl of R_{d}, is optionally substituted with one or more Rₑ;

two of Rₑ group, taken together with the atom(s) to which they are attached, may optionally form a cycloalkyl or heterocycloalkyl, in which said cycloalkyl or heterocycloalkyl of Rₑ is optionally substituted with one or more groups selected from H, D, alkyl, alkenyl, alkynyl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, aryl, or heteroaryl;
each of k, g, m, n, p and q is, independently, 0, 1, 2, 3, 4, or 5;
s is 0 or 1; and
f is 0 or 1,

or a pharmaceutically acceptable salt thereof; dispersed in a solid matrix that comprises (a) at least one pharmaceutically acceptable water-soluble polymeric carrier, (b) at least one pharmaceutically acceptable surfactant, and optionally, and optionally (c) at least one pharmaceutically acceptable antioxidant.

Another aspect of the invention described herein provides a solid orally deliverable dosage form comprising such a solid dispersion, optionally together with one or more additional excipients.

Another aspect of the invention described herein provides a process for preparing a solid dispersion as described above. The process comprises: (a) subjecting to elevated temperature (i) an active pharmaceutical ingredient (API) that comprises a compound of Formula (A) or a pharmaceutically acceptable salt thereof, (ii) a pharmaceutically acceptable water-soluble polymeric carrier, (iii) a pharmaceutically acceptable surfactant, and optionally (iv) a pharmaceutically acceptable antioxidant, to provide an extrudable semi-solid mixture:

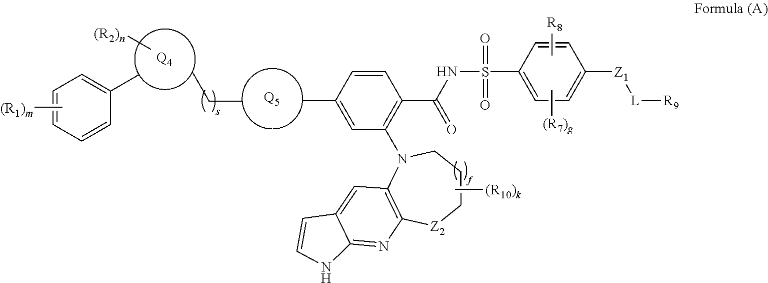

wherein

Q₄ is cycloalkyl, cycloalkenyl, heterocycloalkyl, heterocycloalkenyl, aryl, heteroaryl, or spiro heterocyclic;

Q₅ is cycloalkyl, cycloalkenyl, heterocycloalkyl, heterocycloalkenyl, aryl, heteroaryl, or spiro heterocyclic;

each of R₁, R₂, R₇, R₈, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c},-alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more R_{d};

Rₐ, R_{b}, Rₑ, R_{bb}, R_{cc}, and R_{d}, independently, is H, D, alkyl, spiroalkyl, alkenyl, alkynyl, alkoxy, alkoxyalkyl, haloalkyl, hydroxyalkyl, aminoalkyl, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;

Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;

Z₁ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;

L is-L₁-L₂-;

L₁ is a bond, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally substituted with one or more R_{d};

L₂ is a bond, or an alkyl in which one or more -Lᵢ- are optionally inserted between any two adjacent carbon atoms;

-Lᵢ- is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;

two of R₁ group, taken together with the atoms to which they are attached, may optionally form a cycloalkyl or heterocycloalkyl, in which said cycloalkyl or heterocycloalkyl of R₁, is optionally substituted with one or more R_{d};

two of R₂ group, taken together with the atom(s) to which they are attached, may optionally form a cycloalkyl or heterocycloalkyl, in which said cycloalkyl or heterocycloalkyl of R₂, is optionally substituted with one or more R_{d};

two of R₇ group, taken together with the atoms to which they are attached, may optionally form a cycloalkyl or heterocycloalkyl, in which said cycloalkyl or heterocycloalkyl of R₇, is optionally substituted with one or more R_{d};

two of R₁₀ group, taken together with the atom(s) to which they are attached, may optionally form a cycloalkyl or heterocycloalkyl, in which said cycloalkyl or heterocycloalkyl of R₁₀, is optionally substituted with one or more R_{d};

R₇ and L group, taken together with the atom to which they are attached, may optionally form a cycloalkyl, or heterocycloalkyl, in which said cycloalkyl or heterocycloalkyl of R₇ and L, is optionally substituted with one or more Rₑ;

R_{b} and R, group, taken together with the atom to which they are attached, may optionally form a cycloalkyl, or heterocycloalkyl, in which said cycloalkyl or heterocycloalkyl of R_{b} and R_{c}, is optionally substituted with one or more Rₑ;

two of R_{d} group, taken together with the atom(s) to which they are attached, may optionally form a cycloalkyl, or heterocycloalkyl, in which said cycloalkyl or heterocycloalkyl of R_{d}, is optionally substituted with one or more Rₑ;

two of Rₑ group, taken together with the atom(s) to which they are attached, may optionally form a cycloalkyl or heterocycloalkyl, in which said cycloalkyl or heterocycloalkyl of Rₑ is optionally substituted with one or more groups selected from H, D, alkyl, alkenyl, alkynyl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, aryl, or heteroaryl;
each of k, g, m, n, p and q is, independently, 0, 1, 2, 3, 4, or 5;
s is 0 or 1; and
f is 0 or 1;

(b) extruding the semi-solid mixture; and

(c) cooling the resulting extrudate to provide a solid matrix comprising the polymeric carrier, and the surfactant (and optionally the antioxidant), and having the compound or salt thereof dispersed in an essentially non-crystalline form therein.

As used herein, a “melt” is a liquid or semi-solid (e.g., rubbery) state induced by elevated temperature wherein it is possible for a first component to become homogeneously distributed in a matrix comprising a second component. Typically, the second (matrix) component, for example a polymeric carrier, is in such a state and other components, for example including a compound of Formula (A) or a salt thereof, dissolve in the melt, thus forming a solution.

As used herein, “elevated temperature” is meant a temperature above a softening point of the polymeric carrier, as affected by other components if present, such as plasticizers or surfactants. In certain embodiments, the elevated temperature is about 100° C. to about 200° C., about 125° C. to about 175° C., or about 140-160° C.

Preparation of the Melt can take place in a Variety of Ways. Mixing of the components can take place before, during or after formation of the melt. For example, the components can be mixed first and then subjected to elevated temperature to form the melt; alternatively mixing and melting can take place simultaneously. In one embodiment the polymeric carrier is first melted, optionally with the surfactant component, and the API is then added to the resulting melt. Usually, the melt is thoroughly mixed while at elevated temperature in order to ensure homogeneous dispersion of the API.

A related aspect of the invention provides a solid dispersion prepared by the process described above.

A further related aspect of the invention provides an orally deliverable pharmaceutical dosage form comprising the solid dispersion of the invention.

Another aspect of the invention provides a method for treating a neoplastic, immune or autoimmune disease, comprising orally administering to a subject having the disease a therapeutically effective amount of the solid dispersion of the invention, or one or more solid dosage forms comprising such a dispersion.

Examples of neoplastic diseases include cancers. A specific illustrative type of cancer that can be treated according to the present method is non-Hodgkin's lymphoma (NHL). Another specific illustrative type of cancer that can be treated according to the present method is chronic lymphocytic leukemia (CLL). Yet another specific illustrative type of cancer that can be treated according to the present method is acute lymphocytic leukemia (ALL), for example in a pediatric patient.

Additional embodiments of the invention, including more particular aspects of those provided above, are provided hereinbelow. It should be understood that any embodiment of the invention, including those described only under one aspect of the invention or only in the examples, can be combined with any other embodiments of the invention, unless explicitly disclaimed or would be improper.

### DETAILED DESCRIPTION OF THE INVENTION

The invention described herein provides a solid dispersion comprises an active ingredient in an essentially non-crystalline or amorphous form, which is usually more soluble than the crystalline form.

The term “solid dispersion” herein encompasses systems having small solid-state particles (e.g., essentially non-crystalline or amorphous particles) of one phase dispersed in another solid-state phase. More particularly, the present solid dispersions comprise particles of one or more active ingredients dispersed in an inert carrier or matrix in solid state, and can be prepared by melting (such as hot melt extrusion or HME) or solvent (such as spray-drying) methods or by a combination of melting and solvent methods. According to the present invention, a melt-extrusion method as described herein is preferred.

As used herein, an “amorphous form” refers to a particle without definite structure, i.e., lacking crystalline structure.

As used herein, the term “essentially non-crystalline” means that no more than about 5%, for example no more than about 2% or no more than about 1% crystallinity, is observed by X-ray diffraction analysis. In a particular embodiment, no detectable crystallinity is observed by one or both of X-ray diffraction analysis or polarization microscopy. When no detectable crystallinity is observed, the solid dispersion herein may additionally or alternatively be described as a solid solution.

Thus in one aspect, the invention provides a solid dispersion comprising a compound of Formula (A),

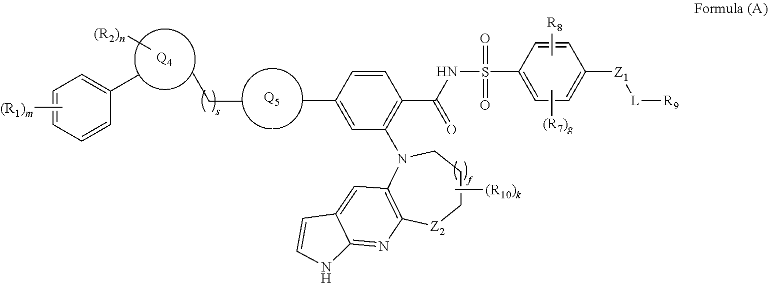

wherein

Q₄ is cycloalkyl, cycloalkenyl, heterocycloalkyl, heterocycloalkenyl, aryl, heteroaryl, or spiro heterocyclic;

Q₅ is cycloalkyl, cycloalkenyl, heterocycloalkyl, heterocycloalkenyl, aryl, heteroaryl, or spiro heterocyclic;

each of R₁, R₂, R₇, R₈, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c},-alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more R_{d};

Rₐ, R_{b}, Rₑ, R_{bb}, R_{cc}, and R_{d}, independently, is H, D, alkyl, spiroalkyl, alkenyl, alkynyl, alkoxy, alkoxyalkyl, haloalkyl, hydroxyalkyl, aminoalkyl, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;

Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;

Z₁ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;

L is-L₁-L₂-;

L₁ is a bond, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally substituted with one or more R_{d};

L₂ is a bond, or an alkyl in which one or more -Lᵢ- are optionally inserted between any two adjacent carbon atoms;

-Lᵢ- is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;

two of R₁ group, taken together with the atoms to which they are attached, may optionally form a cycloalkyl or heterocycloalkyl, in which said cycloalkyl or heterocycloalkyl of R₁, is optionally substituted with one or more R_{d};

two of R₂ group, taken together with the atom(s) to which they are attached, may optionally form a cycloalkyl or heterocycloalkyl, in which said cycloalkyl or heterocycloalkyl of R₂, is optionally substituted with one or more R_{d};

two of R₇ group, taken together with the atoms to which they are attached, may optionally form a cycloalkyl or heterocycloalkyl, in which said cycloalkyl or heterocycloalkyl of R₇, is optionally substituted with one or more R_{d};

two of R₁₀ group, taken together with the atom(s) to which they are attached, may optionally form a cycloalkyl or heterocycloalkyl, in which said cycloalkyl or heterocycloalkyl of R₁₀, is optionally substituted with one or more R_{d};

R₇ and L group, taken together with the atom to which they are attached, may optionally form a cycloalkyl, or heterocycloalkyl, in which said cycloalkyl or heterocycloalkyl of R₇ and L, is optionally substituted with one or more Rₑ;

R_{b} and R, group, taken together with the atom to which they are attached, may optionally form a cycloalkyl, or heterocycloalkyl, in which said cycloalkyl or heterocycloalkyl of R_{b} and R_{c}, is optionally substituted with one or more Rₑ;

two of R_{d} group, taken together with the atom(s) to which they are attached, may optionally form a cycloalkyl, or heterocycloalkyl, in which said cycloalkyl or heterocycloalkyl of R_{d}, is optionally substituted with one or more Rₑ;

two of Rₑ group, taken together with the atom(s) to which they are attached, may optionally form a cycloalkyl or heterocycloalkyl, in which said cycloalkyl or heterocycloalkyl of Rₑ is optionally substituted with one or more groups selected from H, D, alkyl, alkenyl, alkynyl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, aryl, or heteroaryl;
each of k, g, m, n, p and q is, independently, 0, 1, 2, 3, 4, or 5;
s is 0 or 1; and
f is 0 or 1,
   or a pharmaceutically acceptable salt thereof; dispersed in a solid matrix that comprises (a) at least one pharmaceutically acceptable water-soluble polymeric carrier, (b) at least one pharmaceutically acceptable surfactant, and optionally, and optionally (c) at least one pharmaceutically acceptable antioxidant. In certain embodiments, the compound is represented by Formula (A-1):

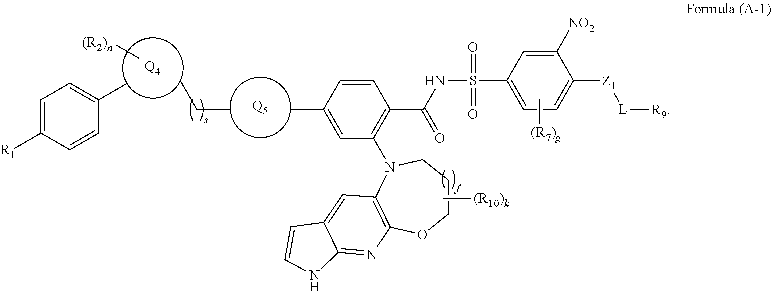

In certain embodiments, the compound is represented by Formula (A-2):

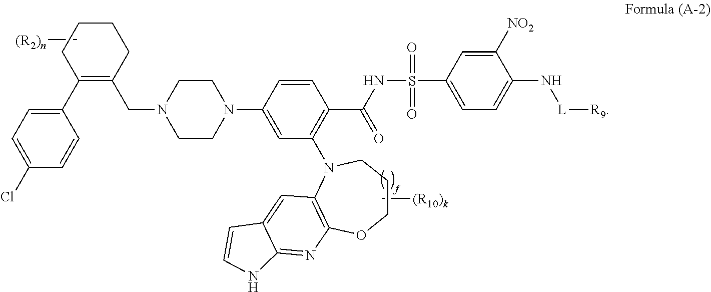

In certain embodiments, the compound is represented by Formula (A-3):

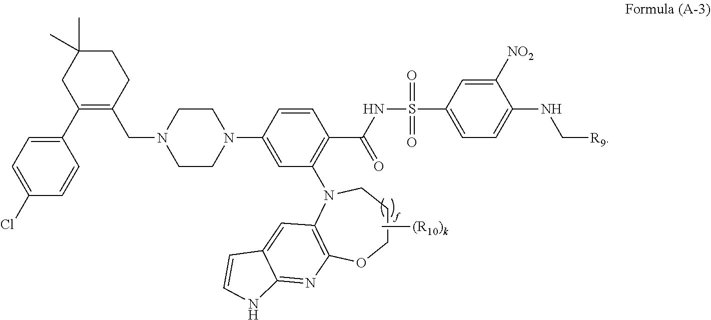

In certain embodiments, the compound is selected from the group consisting of
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
(R)-4-(4-((4′-chloro-2′-fluoro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-2-(4-methyl-3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(4-methyl-3,4-dihydro-2H-pyrrolo[3′,2′:5,6]pyrido[2,3-b][1,4]oxazepin-1(7H)-yl)benzamide,
N-((4-((((R)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-methyl-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-methyl-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-methyl-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-(trifluoromethyl)-3,4-dihydro-2H-pyrrolo[3′,2′:5,6]pyrido[2,3-b][1,4]oxazepin-1(7H)-yl)benzamide,
(S)-N-((4-(((1,4-dioxan-2-yl)methyl)amino)-3-nitrophenyl)sulfonyl)-4-(4-((4′-chloro-4,4-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(S)-N-((4-(((1,4-dioxan-2-yl)methyl)amino)-3-nitrophenyl)sulfonyl)-4-(4-((4′-chloro-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((S)-5-nitro-3-(tetrahydro-2H-pyran-4-yl)-3,4-dihydro-2H-benzo[b][1,4]oxazin-7-yl)sulfonyl)benzamide,
4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((R)-5-nitro-3-(tetrahydro-2H-pyran-4-yl)-3,4-dihydro-2H-benzo[b][1,4]oxazin-7-yl)sulfonyl)benzamide,
N-(((R)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
N-(((S)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
(S)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((5-nitro-3-(tetrahydro-2H-pyran-4-yl)-3,4-dihydro-2H-benzo[b][1,4]oxazin-7-yl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((5-nitro-3-(tetrahydro-2H-pyran-4-yl)-3,4-dihydro-2H-benzo[b][1,4]oxazin-7-yl)sulfonyl)benzamide,
N-(((R)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
N-(((S)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide.

In certain embodiments, the compound or salt is present in the solid dispersion in a parent-compound-equivalent amount of about 5% to about 40% by weight.

As used herein, “parent-compound-equivalent amount” includes the amount of a pharmaceutically acceptable salt of a parent compound that equates the amount of the parent compound in molar.

In certain embodiments, the at least one polymeric carrier comprises homopolymers and copolymers of N-vinyl lactams, cellulose esters, cellulose ethers, high molecular weight polyalkylene oxides, polyacrylates, polymethacrylates, polyacrylamides, vinyl acetate polymers, graft copolymers of polyethylene glycol, polyvinyl caprolactam and polyvinyl acetate, oligo- and polysaccharides, and/or mixtures thereof.

In certain embodiments, the at least one polymeric carrier comprises povidones, copovidones (such as KOLLIDON® VA64 type copovidone), HPMCs, polyethylene glycol/polyvinyl caprolactam/polyvinyl acetate graft copolymers, and/or mixtures thereof; Optionally, the at least one polymeric carrier comprises, consists essentially of, or consists of KOLLIDON® VA64 type copovidone.

In certain embodiments, the at least one surfactant comprises a non-ionic surfactant.

In certain embodiments, the at least one surfactant is a non-ionic surfactant.

In certain embodiments, the at least one surfactant comprises polyoxyethylene glycerides, fatty acid monoesters of sorbitan, polysorbates (such as TWEEN® 80 brand Polysorbate 80 or Polyoxyethylene (20) sorbitan monooleate), a-tocopheryl polyethylene glycol succinate (TPGS) and/or mixtures thereof.

In certain embodiments, the at least one antioxidant comprises ascorbic acid, an ascorbate, a bisulfite, a metabisulfate, a sulfite, curcumin, curcumin derivatives, ursolic acid, resveratrol, resveratrol derivatives, alpha-lipoic acid, thioglycerol, a polyphenol, catachins, grapeseed extract, green tea extract, citric acid, methionine, cysteine, glutathione, tocopherol, propyl gallate, sodium mercaptoacetate, sodium formaldehyde sulfoxylate, ascorbyl palmitate, butylated hydroxyanisole, butylated hydroxytoluene, lecithin, vitamin E, uric acid, and/or mixtures thereof.

In certain embodiments, the at least one antioxidant comprises ascorbic acid or ascorbate.

In certain embodiments, the at least one antioxidant is ascorbic acid or ascorbate.

In certain embodiments, the solid dispersion further comprises at least one glidant.

In certain embodiments, the at least one glidant comprises colloidal silicon dioxide.

In certain embodiments, the compound or salt is present in a parent-compound-equivalent amount of about 5% to about 40% by weight, the at least one polymeric carrier is present in an amount of about 40% to about 85% by weight, the at least one surfactant is present in an amount of about 2.5% to about 20% by weight, and the at least one antioxidant is present in an amount of about 0.25% to about 5% by weight.

In certain embodiments, the compound or salt is present in a parent-compound-equivalent amount of about 5% to about 25% by weight (e.g., about 12-20%, about 15-20%, or about 18%), the at least one polymeric carrier is present in an amount of about 50% to about 80% by weight (e.g., about 60-80%, or about 70-80%), the at least one surfactant is present in an amount of about 2.5% to about 15% by weight (e.g., about 5-10%, or about 7-9%), and the at least one antioxidant is present in an amount of about 0.5% to about 2.5% by weight (e.g., about 0.5-2%, or about 0.5-1%).

In certain embodiments, the solid dispersion further comprises at least one disintegrant (such as 10-30 wt % Croscarmellose Sodium), at least one lubricant (such as 0.2-1.0 wt % Sodium Stearyl Fumarate), and/or at least one coating (such as 2-5 wt % Opadry® II 85F92209-CN Yellow).

In certain embodiments, the compound is
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-(trifluoromethyl)-3,4-dihydro-2H-pyrrolo[3′,2′:5,6]pyrido[2,3-b][1,4]oxazepin-1(7H)-yl)benzamide.

In certain embodiments, the at least one polymeric carrier is a copovidone or a vinylpyrrolidone-vinyl acetate copolymer (such as KOLLIDON® VA64 brand/type copovidone). As used herein, “KOLLIDON® VA64” refers to KOLLIDON® VA64 type copovidone that has substantially the same physical, chemical, and/or biological property as the specific brand of copovidone (i.e., KOLLIDON® brand) used in the formulations of the invention. For the sake of simplicity, the term includes (but is not limited to) the specific brand of copovidone (i.e., KOLLIDON® brand) used in the formulations of the invention.

The same applies to any other brand-named or trademarked products such as TWEEN© 80 brand and other KOLLIDON® brand products.

In certain embodiments, the at least one surfactant is a polysorbate (such as Polysorbate 80 type surfactant).

In certain embodiments, the at least one antioxidant is ascorbic acid or sodium ascorbate.

In certain embodiments, the solid dispersion further comprises at least one glidant.

In certain embodiments, the at least one glidant comprises colloidal silicon dioxide.

In certain embodiments, the solid dispersion is prepared using hot-melt extrusion (HME), or wherein the solid dispersion is a hot-melt-extrusion (HME) formulation.

In certain embodiments, the solid dispersion comprises any one of the formulations of Examples 7-24 and 26-29, and wherein the API is any one of the compounds recited herein, including any one of the following compounds:
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-(trifluoromethyl)-3,4-dihydro-2H-pyrrolo[3′,2′:5,6]pyrido[2,3-b][1,4]oxazepin-1(7H)-yl)benzamide.

In certain embodiments, the solid dispersion exhibits an AUC₍ₒ₋ₜ₎ value of at least about 25,000-150,000 h*ng/ml, at least about 30,000-100,000 h*ng/ml, at least about 40,000-80,000 h*ng/ml, or at least about 50,000-60,000 h*ng/ml when a 100 mg oral dose of said solid dispersion is administered by gavage to a 5-10 kg Beagle dog.

Another aspect of the invention provides a process for preparing a solid dispersion, comprising: (a) subjecting to elevated temperature (i) an active pharmaceutical ingredient (API) that comprises a compound of Formula (A) or a pharmaceutically acceptable salt thereof, (ii) a pharmaceutically acceptable water-soluble polymeric carrier, (iii) a pharmaceutically acceptable surfactant, and optionally, and optionally (iv) a pharmaceutically acceptable antioxidant, to provide an extrudable semi-solid mixture:

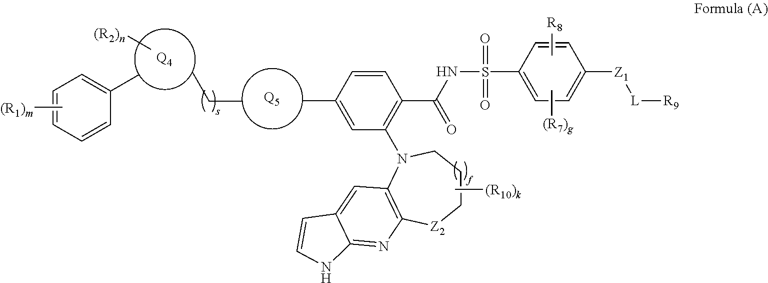

wherein

Q₄ is cycloalkyl, cycloalkenyl, heterocycloalkyl, heterocycloalkenyl, aryl, heteroaryl, or spiro heterocyclic;

Q₅ is cycloalkyl, cycloalkenyl, heterocycloalkyl, heterocycloalkenyl, aryl, heteroaryl, or spiro heterocyclic;

each of R₁, R₂, R₇, R₈, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c},-alkyl-P(O)R_{b}R_{c}, —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more R_{d};

Rₐ, R_{b}, R_{c}, R_{bb}, R_{cc}, and R_{d}, independently, is H, D, alkyl, spiroalkyl, alkenyl, alkynyl, alkoxy, alkoxyalkyl, haloalkyl, hydroxyalkyl, aminoalkyl, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;

Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;

Z₁ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;

L is-L₁-L₂-;

L₁ is a bond, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally substituted with one or more R_{d};

L₂ is a bond, or an alkyl in which one or more -Lᵢ- are optionally inserted between any two adjacent carbon atoms;

-Lᵢ- is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;

two of R₁ group, taken together with the atoms to which they are attached, may optionally form a cycloalkyl or heterocycloalkyl, in which said cycloalkyl or heterocycloalkyl of R₁, is optionally substituted with one or more R_{d};

two of R₂ group, taken together with the atom(s) to which they are attached, may optionally form a cycloalkyl or heterocycloalkyl, in which said cycloalkyl or heterocycloalkyl of R₂, is optionally substituted with one or more R_{d};

two of R₇ group, taken together with the atoms to which they are attached, may optionally form a cycloalkyl or heterocycloalkyl, in which said cycloalkyl or heterocycloalkyl of R₇, is optionally substituted with one or more R_{d};

two of R₁₀ group, taken together with the atom(s) to which they are attached, may optionally form a cycloalkyl or heterocycloalkyl, in which said cycloalkyl or heterocycloalkyl of R₁₀, is optionally substituted with one or more R_{d};

R₇ and L group, taken together with the atom to which they are attached, may optionally form a cycloalkyl, or heterocycloalkyl, in which said cycloalkyl or heterocycloalkyl of R₇ and L, is optionally substituted with one or more Rₑ;

R_{b} and R, group, taken together with the atom to which they are attached, may optionally form a cycloalkyl, or heterocycloalkyl, in which said cycloalkyl or heterocycloalkyl of R_{b} and R_{c}, is optionally substituted with one or more Rₑ;

two of R_{d} group, taken together with the atom(s) to which they are attached, may optionally form a cycloalkyl, or heterocycloalkyl, in which said cycloalkyl or heterocycloalkyl of R_{d}, is optionally substituted with one or more Rₑ;

two of Rₑ group, taken together with the atom(s) to which they are attached, may optionally form a cycloalkyl or heterocycloalkyl, in which said cycloalkyl or heterocycloalkyl of Rₑ is optionally substituted with one or more groups selected from H, D, alkyl, alkenyl, alkynyl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, aryl, or heteroaryl;
each of k, g, m, n, p and q is, independently, 0, 1, 2, 3, 4, or 5;
s is 0 or 1; and
f is 0 or 1;
   (b) extruding the semi-solid mixture; and
   (c) cooling the resulting extrudate to provide a solid matrix comprising the polymeric carrier and the surfactant and having the compound or salt thereof dispersed in an essentially non-crystalline form therein.
   In certain embodiments, the compound is represented by Formula (A-1):

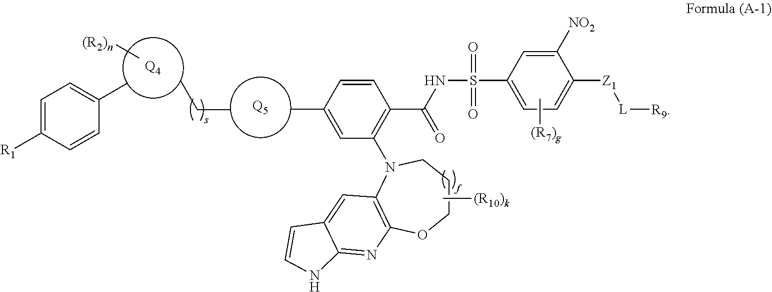

In certain embodiments, the compound is represented by Formula (A-2):

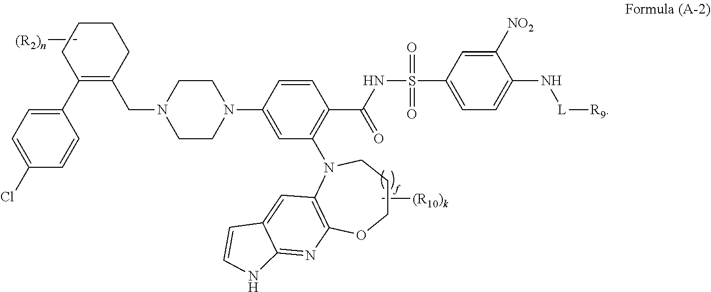

In certain embodiments, the compound is represented by Formula (A-3):

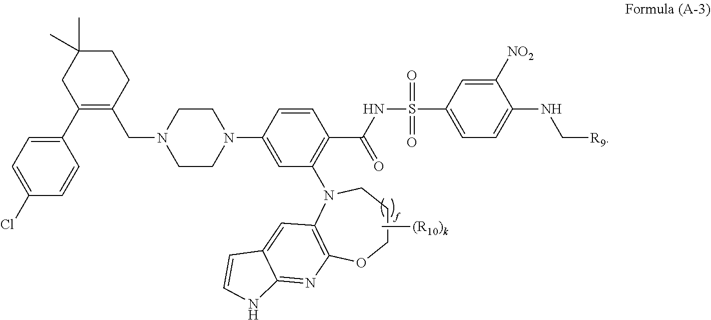

In certain embodiments, the API, polymeric carrier, surfactant, and antioxidant are mixed together before said subjecting to elevated temperature.

In certain embodiments, the API, polymeric carrier, surfactant, and antioxidant are mixed together while subjecting to elevated temperature.

In certain embodiments, the elevated temperature is about 100° C. to about 200° C.

In certain embodiments, the elevated temperature is about 125° C. to about 175° C., or about 140-160° C.

In certain embodiments, the process further comprises calendering the extrudate before or while cooling.

In certain embodiments, the polymeric carrier comprises a copovidone, such as a KOLLIDON® VA64 type copovidone.

In certain embodiments, the surfactant comprises a polysorbate, such as a TWEEN 80 type polysorbate 80.

In certain embodiments, the antioxidant comprises ascorbic acid or sodium ascorbate.

Another aspect of the invention provides an orally deliverable pharmaceutical dosage form comprising the solid dispersion of the invention described herein.

Another aspect of the invention provides a method for treating a neoplastic, immune or autoimmune disease, comprising orally administering to a subject having the disease a therapeutically effective amount of the solid dispersion of the invention.

In certain embodiments, the disease is a neoplastic disease, such as cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, non-Hodgkin's lymphoma, Hodgkin's lymphoma, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma and combinations thereof.

In certain embodiments, the neoplastic disease is chronic lymphocytic leukemia or acute lymphocytic leukemia.

In certain embodiments, the neoplastic disease is non-Hodgkin's lymphoma or Hodgkin's lymphoma.

In certain embodiments, the disease is an immune or autoimmune disease.

In certain embodiments, the solid dispersion is administered in a parent-compound-equivalent dose of about 10 to about 1,000 mg per day of the compound of Formula A or salt thereof at an average treatment interval of about 6 hours to about 7 days.

In certain embodiments, the compound is selected from the group consisting of
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′0.5,6]pyrido[2,3-b][1,4]oxazin-1(6H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
(R)-4-(4-((4′-chloro-2′-fluoro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-2-(4-methyl-3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(4-methyl-3,4-dihydro-2H-pyrrolo[3′,2′:5,6]pyrido[2,3-b][1,4]oxazepin-1(7H)-yl)benzamide,
N-((4-((((R)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-methyl-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-methyl-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-methyl-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-N-((4-(((1,4-dioxan-2-yl)methyl)amino)-3-nitrophenyl)sulfonyl)-4-(4-((4′-chloro-4,4-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(S)-N-((4-(((1,4-dioxan-2-yl)methyl)amino)-3-nitrophenyl)sulfonyl)-4-(4-((4′-chloro-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((S)-5-nitro-3-(tetrahydro-2H-pyran-4-yl)-3,4-dihydro-2H-benzo[b][1,4]oxazin-7-yl)sulfonyl)benzamide,
4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((R)-5-nitro-3-(tetrahydro-2H-pyran-4-yl)-3,4-dihydro-2H-benzo[b][1,4]oxazin-7-yl)sulfonyl)benzamide,
N-(((R)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
N-(((S)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
(S)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((5-nitro-3-(tetrahydro-2H-pyran-4-yl)-3,4-dihydro-2H-benzo[b][1,4]oxazin-7-yl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((5-nitro-3-(tetrahydro-2H-pyran-4-yl)-3,4-dihydro-2H-benzo[b][1,4]oxazin-7-yl)sulfonyl)benzamide,
N-(((R)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
N-(((S)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide.

With the general aspect of the invention having been described, the following sections provide more detailed descriptions for the various aspects of the invention.

### Part A: Chemical Synthesis.

In one embodiment, the active pharmaceutical ingredient (or API) present in the solid dispersion is a compound of Formula (A).

In an alternative embodiment, the API of the solid dispersion of the invention is one selected from compounds specifically identified in WO/2017/132474, WO/2019/040550, WO/2019/040573, PCT/US2019/047404 and PCT/US2019/047403, and pharmaceutically acceptable salts of such compounds, independently of whether these compounds are individually embraced by the present Formula A. Compounds in these Examples, and illustrative procedures for their synthesis, are reproduced hereinbelow.

In a further embodiment, the API present in the solid dispersion is selected from Compounds and pharmaceutically acceptable salts thereof, but only to the extent that such Examples are individually embraced by the present Formula A. The entire disclosure of WO/2017/132474, WO/2019/040550, WO/2019/040573, PCT/US2019/047404 and PCT/US2019/047403 are expressly incorporated herein by reference.

Description of the synthesis of representative compounds is given below. Other compounds of Formula A can be prepared by substantially analogous methods, as will be clear to one of skill in the art. Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### INT-1: Preparation of 1-((4′-chloro-5,5-dimethyl-3,4,5,6-tetrahydro-[1,1′-biphenyl]-2-yl)methyl)piperazine

Synthesis of 2-bromo-4,4-dimethylcyclohex-1-enecarbaldehyde 2: A solution of anhydrous chloroform (57 ml) and anhydrous N,N-dimethylformamide (9 mL) were cooled to ˜3° C. (internal temperature) under nitrogen before phosphorus tribromide (10 mL, 0.1 mol) was introduced dropwise at a rate so that the reaction was maintained at ˜3° C. After the addition was complete the reaction was allowed to warm slowly to ˜10° C. and then the temperature was raised to 70° C. where it was maintained for 30 min. The reaction was cooled to rt and 3,3-dimethylcyclohexanone 1 (5 g, 0.04 mol) was added slowly over 20 min. After the addition was complete the reaction was warmed to 70° C. and it was stirred for 1.5 h. The mixture was then cooled to 0° C. and a solution of 4M sodium acetate (53 ml) was added slowly. The pH of the resulting solution was adjusted to -7 using a solution of 5M NaOH and the mixture was then extracted with heptanes (100 mL×3). The combined organic fractions were dried (Na₂SO₄), filtered and concentrated under reduced pressure to give 2-bromo-4,4-dimethylcyclohex-1-enecarbaldehyde 2 (4 g, 49%) as a yellow oil.

### Synthesis of 2-(4-chlorophenyl)-4,4-dimethylcyclohex-1-enecarbaldehyde 3: To a degassed solution of 2-bromo-4,4-dimethylcyclohex-1-enecarbaldehyde 2 (5 g, 0.023 mol) and 4-chlorophenyl boronic acid (3.6 g, 0.023 mol) in 1,4-dioxane (50 mL) at rt was added a solution of 2M Na₂CO₃ (20.4 ml). Nitrogen was bubbled through the mixture for 2 min and then PdCl₂(dppf) (0.5 g) was added. The reaction flask was heated to 120° C. where it was maintained for 3 h. After this time the suspension was cooled to rt and filtered through Celite. The collected solids were washed with additional dichloromethane and the combined filtrate and washings were concentrated under reduced pressure. Purification by column chromatography on silica with PE: EA=20:1 gave 2-(4-chlorophenyl)-4,4-dimethylcyclohex-1-enecarbaldehyde 3 (3 g, 53%) as a white solid. MS: 249[M+H]⁺

Synthesis of (2-(4-chlorophenyl)-4,4-dimethylcyclohex-1-enyl)methanol 4: A solution of 2-(4-chlorophenyl)-4,4-dimethylcyclohex-1-enecarbaldehyde 3 (20 g, 80.6 mmol) in MeOH (100 mL) was cooled to 0° C., NaBH₄ (3.1 g, 80.6 mmol) was added portionwise to the reaction at a rate so that the reaction was maintained at 0-5° C. After added, the mixture was stirred for 1 h at 0° C. Water was added slowly to the mixture and extracted with EA (200 mL×3), the organic layer was washed with brine and dried Na₂SO₄, filtered and concentrated under reduced pressure to give (2-(4-chlorophenyl)-4,4-dimethylcyclohex-1-enyl)methanol 4 (15 g, 75%) as a white solid. MS: 233[M+H−H₂O]⁺.

### Synthesis of 1-(2-(bromomethyl)-5,5-dimethylcyclohex-1-enyl)-4-chlorobenzene 5: A solution of (2-(4-chlorophenyl)-4,4-dimethylcyclohex-1-enyl)methanol 4 (15 g, 0.060 mol) and in Et₂O (300 ml) was cooled to 0° C. before phosphorus tribromide (7.5 mL) was added dropwise to the mixture, after added, the mixture was stirred for 1 h at 0° C. for 90 minutes. The reaction mixture was added H₂O before being extracted with EA. The organic layer was washed with a saturated NaHCO₃ solution and brine and dried Na₂SO₄, filtered and concentrated under reduced pressure to give 1-(2-(bromomethyl)-5,5-dimethylcyclohex-1-enyl)-4-chlorobenzene 5 (18 g, 96%) as a colorless oil

Synthesis of tert-butyl 4-((2-(4-chlorophenyl)-4,4-dimethylcyclohex-1-enyl)methyl)piperazine-1-carboxylate-To a solution of 1-bromo-2-(bromomethyl)-5,5-dimethylcyclohex-1-ene 5 (21 g, 0.067 mol) and tert-butyl piperazine-1-carboxylate (12.4 g, 0.067 mol) in dichloromethane (200 ml) at rt was added TEA (12.2 g, 0.12 mol). The reaction was stirred for 2 h. The reaction mixture was concentrated under reduced pressure to give the crude product. Purification by column chromatography on silica with PE:EA=20:1 provided tert-butyl 4-((2-(4-chlorophenyl)-4,4-dimethylcyclohex-1-enyl)methyl)piperazine-1-carboxylate 6 (21 g, 75%).

Synthesis of 1-((2-(4-chlorophenyl)-4,4-dimethylcyclohex-1-enyl)methyl)piperazine hydrogen chloride: To a solution of tert-butyl 4-((2-(4-chlorophenyl)-4,4-dimethylcyclohex-1-enyl)methyl)piperazine-1-carboxylate 6 (30 g, 0.072 mol) in MeOH (20 ml) was added conc. HCl (50 mL). The reaction was stirred for 24 hours and then concentrated under reduced pressure. A saturated solution of Na₂CO₃ was added to adjust the pH to -8-9 and the mixture was extracted with dichloromethane (x2). The combined extracts were washed with brine, dried (Na₂SO₄), filtered and concentrated under reduced pressure. The oil product was treated with MeOH/HCl(g) (3M, 500 mL) and stirred for 1 hour, then concentrated under reduced pressure to get the product 1-((2-(4-chlorophenyl)-4,4-dimethylcyclohex-1-enyl)methyl)piperazine hydrogen chloride IM-14-1 (23 g, 83%). MS: 319[M+H]⁺ ¹H NMR (400 MHz, DMSO) δ 11.51 (s, 1H), 9.60 (s, 1H), 9.18 (s, 1H), 7.45 (d, J=8.2 Hz, 2H), 7.15 (d, J=8.0 Hz, 2H), 3.43 (s, 8H), 2.84 (s, 2H), 2.39 (s, 2H), 2.03 (s, 2H), 1.45 (t, J=6.0 Hz, 2H), 0.96 (s, 6H).

### INT-2: Preparation of 3-nitro-4-(((tetrahydro-2H-pyran-4-yl)methyl)amino)benzenesulfonamide

To a 500 mL three-neck RB flask equipped with a mechanical stirrer were charged the 4-chloro-3-nitrobenzenesulfonamide (23.7 g, 100 mmol), DIPEA (12.9 g, 100 mmol), (tetrahydro-2H-pyran-4-yl)methanamine(11.5 g, 100 mmol) and acetonitrile (200 mL). The reaction mixture was adjusted to an internal temperature of 80° C. and agitated for no less than 12 hours. The product solution was cooled down to 40° C. and agitated for no less than 1 hour until precipitation observed. The product slurry was further cooled to 20° C. Water (80 mL) was slowly charged over no less than 1 hour, and the mixture cooled to 10° C. and agitated for no less than 2 hours before collected by filtration. The wet cake was washed with 1:1 mix of acetonitrile:water (40 mL). The wet cake was rinsed with water (80 mL) at 40° C. for no less than 1 hour before collected by filtration. The wet cake was rinsed with water (20 mL), and dried at 750° C. under vacuum to give the 3-nitro-4-(((tetrahydro-2H-pyran-4-yl)methyl)amino)benzenesulfonamide (24.5 g, 78%) as an orange solid. ¹H NMR (400 MHz, DMSO) δ 8.60 (t, J=5.9 Hz, 1H), 8.48 (d, J=2.2 Hz, 1H), 7.84 (dd, J=9.2, 2.0 Hz, 1H), 7.54-7.18 (m, 3H), 3.86 (dd, J=11.3, 3.2 Hz, 2H), 3.35 (s, 2H), 3.27 (t, J=10.9 Hz, 2H), 1.92 (ddd, J=11.2, 7.4, 3.9 Hz, 1H), 1.62 (d, J=11.4 Hz, 2H), 1.27 (qd, J=12.3, 4.4 Hz, 2H).

### INT-3: Preparation of 4-[[(4-fluorooxan-4-yl)methyl]amino]-3-nitrobenzene-1-sulfonamide

Into a 50-mL round-bottom flask, was placed (4-fluorooxan-4-yl)methanamine hydrochloride (500 mg, 2.95 mmol, 1.00 equiv), 4-fluoro-3-nitrobenzene-1-sulfonamide (650 mg, 2.95 mmol, 1.00 equiv), tetrahydrofuran (15 mL), Cs₂CO₃ (2.8 g, 8.59 mmol, 3.00 equiv). The resulting solution was stirred for 14 h at 50° C. in an oil bath. The reaction mixture was cooled to room temperature. The resulting mixture was filtered and concentrated under vacuum. The residue was applied onto a silica gel column with ethyl acetate/petroleum ether (4:1). This resulted in 650 mg (66%) of 4-[[(4-fluorooxan-4-yl)methyl]amino]-3-nitrobenzene-1-sulfonamide as a yellow solid. LCMS (ES, m/z): M+1: 334. H-NMR: (300 MHz, DMSO, ppm): δ8.58 (t, J=6.3 Hz, 1H), 8.49 (d, J=2.1 Hz, 1H), 7.90-7.80 (m, 1H), 7.44 (d, J=9.3 Hz, 1H), 7.34 (s, 2H), 3.87-3.70 (m, 4H), 3.61-3.50 (m, 2H), 1.95-1.70 (m, 4H).

### INT-4: Preparation of (S)-4-((1,4-dioxan-2-yl)methylamino)-3-nitrobenzenesulfonamide Synthesis of (R)-1-chloro-3-(2-chloroethoxy)propan-2-ol: (R)-2-(chloromethyl)oxirane (500.0 g, 5.4 mol, 1.00 equiv) was slowly added to a stirred solution of 2-chloroethanol (870.0, 10.8 mol, 2.00 equiv) and BF₃.Et₂O (38.0 g, 27 mmol, 0.05 equiv) at 45° C. The reaction mixture was heated on an oil bath for 3 h at 45° C. The reaction mixture was cooled to R,T and Diethyl ether (100 mL) was added to this solution. The organic layer was washed with water (2×300 mL), dried over magnesium sulfate, and concentrated to yield a light brown liquid (R)-1-chloro-3-(2-chloroethoxy)propan-2-ol (800.0 g, quantitative). H-NMR: (300 MHz, DMSO-d₆,ppm) δ: 3.85-3.47 (m, 9H)

Synthesis of (R)-2-((2-chloroethoxy)methyl)oxirane. (R)-1-chloro-3-(2-chloroethoxy)propan-2-ol (800.0 g, crude, 4.7 mol, 1.0 eq) was added dropwise to a stirred solution of NaOH (465.0 g, 11.6 mol, 2.5 eq) in water (500 mL) on an ice-bath. The ice-bath was immediately removed after addition of (R)-1-chloro-3-(2-chloroethoxy)propan-2-ol. After stirring 2 h at an ambient temperature, diethyl ether (1.5 L) and water (500 mL) were added. The organic layer was washed with water (1×50 mL), dried over sodium sulfate, and concentrated to give a light brown liquid (R)-2-((2-chloroethoxy)methyl)oxirane (400.0 g). H-NMR: (300 MHz, CDCL3, ppm) δ:3.82-3.52 (m, 5H), 3.40-3.35 (m, 1H), 3.11-3.09 (m, 1H), 2.75-2.73.

Synthesis of (S)-(1,4-dioxan-2-yl)methanol. (R)-2-((2-chloroethoxy)methyl)oxirane (400.0 g, 2.94 mol, 1.0 eq) was added to a solution of NaOH (294.0 g, 7.35 mol, 2.5 eq) in water (2900 mL) at room temperature. The reaction mixture was heated on an oil bath for 2 h at 90° C. The resulting solution was cooled to R,T and adjusted PH value to 5 by HCl (6 M). The mixture was concentrated and the residue was distilled (90-95° C., 0.1 kPa) under vacuum pump to give a colorless oil (S)-(1,4-dioxan-2-yl)methanol (110 g, 31.7%). H-NMR: (300 MHz, CDCL3, ppm) δ: 3.85-3.42 (m, 9H), 2.15 (bs, 1H).

Synthesis of (R)-(1,4-dioxan-2-yl)methyl methanesulfonate. A mixture of (S)-(1, 4-dioxan-2-yl)methanol (50.0 g, 0.42 mol, 1.0 eq), TEA (63.6 g, 0.63 mol, 1.5 eq) and DCM (500 mL) at ice-bath, MsCl (48.1 g, 0.42 mol, 1.0 eq) was added dropwise. And then, the ice-bath removed and the mixture was stirred at R,T for 2 hours. The reaction mixture was washed by water (2×50 mL) and the organic phase was dried over sodium sulfate, and concentrated to give a light brown oil (R)-(1,4-dioxan-2-yl)methyl methanesulfonate (71.0 g, 83%). H-NMR: (300 MHz, CDCL3, ppm) δ:4.23-4.20 (m, 2H), 3.82-3.56 (m, 6H), 3.50-3.40 (m, 1H), 3.02 (m, 3H).

Synthesis of (S)-(1,4-dioxan-2-yl)methanamine: In 1000 mL autoclave, to a solution of (R)-(1,4-dioxan-2-yl)methyl methanesulfonate (70.0 g, 0.36 mol, 1.0 eq) in NH₃.MeOH (7 M, 500 mL) was stirred at 80° C. for 12 hours, the reaction mixture was cooled to R,T and concentrated to give a light brown oil (S)-(1,4-dioxan-2-yl)methanamine (30.0 g, 73%). NMR: (300 MHz, DMSO-d₆,ppm) δ: 8.27 (bs, 2H), 3.82-3.42 (m, 6H), 3.24-3.20 (m, 1H), 2.98-2.62 (m, 2H).

### Synthesis of (S)-4-((1,4-dioxan-2-yl)methylamino)-3-nitrobenzenesulfonamide: A mixture of (S)-(1, 4-dioxan-2-yl)methanamine (25.0 g, 0.21 mol, 1.0 eq), 4-fluoro-3-nitrobenzenesulfonamide (46.0 g, 0.21 mol, 1.0 eq) and Cs₂CO₃ (137.3 g, 0.42 mol, 2.0 eq) in THE (700 mL) was stirred at 50° C. for 6 hours, LCMS showed material was consumed completely, the reaction mixture was cooled R,T and poured into water (3500 mL). The mixture was filtrated and collected filtrate cake and dried by oven to give a yellow solid (S)-4-((1,4-dioxan-2-yl)methylamino)-3-nitrobenzenesulfonamide (60.0 g, 89.5%). H-NMR: (300 MHz, DMSO-d₆,ppm) δ: 8.52-8.47 (m, 2H), 7.86-7.83 (m, 1H), 7.28-7.00 (m, 3H), 3.82-3.29 (m, 9H)

### INT-5: Preparation of methyl 2-bromo-4-(4-[[2-(4-chlorophenyl)-4,4-dimethylcyclohex-1-en-1-yl]methyl]piperazin-1-yl)benzoate

Into a 250-mL round-bottom flask, was placed a solution of Example 1-1, i.e, 1-[[2-(4-chlorophenyl)-4,4-dimethylcyclohex-1-en-1-yl]methyl]piperazine (15.09 g, 47.32 mmol, 1.00 equiv) in DMA (150 mL), DIEA (12.9 g, 99.81 mmol, 2.00 equiv), methyl 2-bromo-4-fluorobenzoate (11.6 g, 49.78 mmol, 1.00 equiv). The resulting solution was stirred for 12 h at 100 degree. The reaction mixture was cooled to room temperature. The reaction was then quenched by the addition of 50 mL of water. The resulting solution was extracted with 3×100 mL of ethyl acetate and the organic layers combined. The resulting mixture was washed with 3×100 mL of brine. The mixture was dried over anhydrous sodium sulfate, then filtered and concentrated under vacuum. The residue was applied onto a silica gel column with ethyl acetate/petroleum ether (0:1-1:5). This resulted in 7 g (crude) of methyl 2-bromo-4-(4-[[2-(4-chlorophenyl)-4,4-dimethylcyclohex-1-en-1-yl]methyl]piperazin-1-yl)benzoate as yellow oil. LC-MS: (ES, m/z): M+1=533, 531.

### INT-6: Preparation of 2-bromo-4-(4-((2-(4-chlorophenyl)-4,4-dimethylcyclohex-1-enyl)methyl)piperazin-1-yl)-N-(3-nitro-4-((tetrahydro-2H-pyran-4-yl)methylamino)phenylsulfonyl)benzamide

### Synthesis of methyl 2-bromo-4-(4-((2-(4-chlorophenyl)-4,4-dimethylcyclohex -1-enyl) methyl)piperazin-1-yl)benzoate Into a 20000-mL round-bottom flask, was placed 1-((2-(4-chlorophenyl)-4, 4-dimethylcyclohex-1-enyl)methyl)piperazine dihydrochloride (600 g, 1.53 mol, 1 equiv), methyl 2-bromo-4-fluorobenzoate (357 g, 1.53 mol, 1 equiv), DBU (319 g, 6.12 mol, 4 equiv) and DMSO (8000 mL). The resulting solution was stirred for 20 h at 70 degrees C. LCMS showed material was completely consumed. The resulting mixture was cooled to R,T and poured into water (32 L). The mixture was filtrated, collection of filter cake and the filter cake was washed by water (3000 mL×3) and dried by oven to give product 740 g (Y: 91%) methyl 2-bromo-4-(4-((2-(4-chlorophenyl)-4,4-dimethylcyclohex-1-enyl)methyl)piperazin-1-yl)benzoate as a white solid. H-NMR-PH-PHNW-4-55-400: (300 MHz, DMSO-d₆,ppm) δ: 7.73 (d, J=9.0 Hz, 1H), 7.42-7.39 (m, 2H), 7.18-7.12 (m, 3H), 6.97-6.94 (m, 1H), 4.00-3.84 (m, 2H), 3.76 (s, 2H), 3.57 (s, 3H), 3.51-3.33 (m, 4H), 2.79-2.60 (m, 2H), 2.32-2.30 (m, 2H), 2.03-1.97 (m, 2H), 1.47-1.45 (m, 2H), 0.96 (s, 6H)

Synthesis of 2-bromo-4-(4-((2-(4-chlorophenyl)-4,4-dimethylcyclohex-1-enyl) methyl)piperazin-1-yl)benzoic acid: Into a 20000-mL round-bottom flask, was placed methyl 2-bromo-4-(4-((2-(4-chlorophenyl)-4,4-dimethylcyclohex-1-enyl)methyl)piperazin-1-yl)benzoate (730 g, 1.37 mol, 1 equiv), LiOH (131.5 g, 5.48 mol, 4 equiv) and MeOH/THF/water (4500 mL/3000 mL/1000 mL). The resulting solution was stirred for 16 h at 70 degrees C. LCMS showed material was completely consumed. The resulting mixture was cooled to R,T and concentrated. The residue was diluted with water (5000 mL) and the mixture was adjust PH to 3-5 with HCl (6 M), followed by filtrated, collection of filter cake and dried by oven to give product 650 g (Y: 93%) 2-bromo-4-(4-((2-(4-chlorophenyl)-4,4-dimethylcyclohex-1-enyl)methyl)piperazin-1-yl)benzoic acid as a white solid. H-NMR-PH-PHNW-4-55-400: (300 MHz, DMSO-d₆,ppm) δ:10.60 (bs, 1H), 7.73 (d, J=8.4 Hz, 1H), 7.42-7.39 (m, 2H), 7.14-7.11 (m, 3H), 6.95-6.92 (m, 1H), 4.00-3.84 (m, 2H), 3.76 (s, 2H), 3.51-3.33 (m, 4H), 2.79-2.60 (m, 2H), 2.32-2.30 (m, 2H), 2.03-1.97 (m, 2H), 1.47-1.45 (m, 2H), 0.97 (s, 6H).

### Synthesis of 2-bromo-4-(4-((2-(4-chlorophenyl)-4,4-dimethylcyclohex-1-enyl) methyl)piperazin-1-yl)-N-(3-nitro-4-((tetrahydro-2H-pyran-4-yl)methylamino)phenylsulfonyl)benzamide: Into a 20000-mL round-bottom flask, was placed 2-bromo-4-(4-((2-(4-chlorophenyl)-4,4-dimethylcyclohex-1-enyl)methyl)piperazin-1-yl)benzoic acid (583 g, 1.13 mol, 1 equiv), DCM (10 L), 3-nitro-4-[[(oxan-4-yl)methyl]amino]benzene-1-sulfonamide (338 g, 1.07 mol, 0.95 equiv), EDCI (326 g, 1.7 mol, 1.5 equiv), DMAP (551 g, 4.52 mol, 4 equiv). The resulting solution was stirred for overnight at 25 degrees C. LCMS showed material was completely consumed. The resulting mixture is followed by dilute hydrochloric acid (1.0 M) (1000 mL×3), saturated sodium bicarbonate (1000 mL×3) and brine (1000 mL×1), and then the organic phase was dried by Na₂SO₄, filtrated. The filtrate was concentrated to give product 857 g (Y: 93%) as a light brown yellow solid 2-bromo-4-(4-((2-(4-chlorophenyl)-4,4-dimethylcyclohex-1-enyl)methyl)piperazin-1-yl)-N-(3-nitro-4-((tetrahydro-2H-pyran-4-yl)methylamino)phenylsulfonyl)benzamide as a brown yellow solid. LC-MS: (ES, m/z): M+1=814/816/818, R,T=2.01 min. H-NMR-PH-PHNW-4-55-400: (300 MHz, DMSO-d₆, ppm) δ: 8.63-8.61 (m, 2H), 7.94-7.92 (m, 1H), 7.37-7.35 (m, 3H), 7.27-7.24 (m, 1H), 7.05-7.02 (m, 3H), 6.86-6.83 (m, 1H), 3.87-3.82 (m, 2H), 3.37-3.23 (m, 8H), 2.92 (s, 2H), 2.50-2.38 (m, 4H), 2.22-2.20 (m, 2H), 2.00-1.97 (m, 2H), 1.64-1.60 (m, 2H), 1.48-1.46 (m, 2H), 1.26-1.20 (m, 2H), 0.97 (s, 6H)

### Compound 2-1: Preparation of 4-(4-((4′-chloro-5,5-dimethyl-3,4,5,6-tetrahydro-[1,1′-biphenyl]-2-yl)methyl)piperazin-1-yl)-2-(2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide

Into a 250-mL round-bottom flask, was placed a solution of 1-[[2-(4-chlorophenyl)-4,4-dimethylcyclohex-1-en-1-yl]methyl]piperazine (15.09 g, 47.32 mmol, 1.00 equiv) in DMA (150 mL), DIEA (12.9 g, 99.81 mmol, 2.00 equiv), methyl 2-bromo-4-fluorobenzoate (11.6 g, 49.78 mmol, 1.00 equiv). The resulting solution was stirred for 12 h at 100 degree. The reaction mixture was cooled to room temperature. The reaction was then quenched by the addition of 50 mL of water. The resulting solution was extracted with 3×100 mL of ethyl acetate and the organic layers combined. The resulting mixture was washed with 3×100 mL of brine. The mixture was dried over anhydrous sodium sulfate, then filtered and concentrated under vacuum. The residue was applied onto a silica gel column with ethyl acetate/petroleum ether (0:1-1:5). This resulted in 7 g (crude) of methyl 2-bromo-4-(4-[[2-(4-chlorophenyl)-4,4-dimethylcyclohex-1-en-1-yl]methyl]piperazin-1-yl)benzoate as yellow oil. LC-MS (ES, m/z): M+1=533, 531.

Into a 40-mL round-bottom flask, was placed a solution of 4-[[2-(trimethylsilyl)ethoxy]methyl]-13-oxa-2,4,10-triazatricyclo[7.4.0.0\hat{ }[3,7]]trideca-1,3(7),5,8-tetraene (175 mg, 0.57 mmol, 1.00 equiv) in dioxane (10 mL), Cs₂CO₃ (560 mg, 1.72 mmol, 3.00 equiv), XantPhos Pd G2 (CAS: 1375325-77-1) (53 mg, 0.06 mmol, 0.10 equiv), methyl 2-bromo-4-(4-[2-(4-chlorophenyl)-4,4-dimethylcyclohex-1-en-1-yl]methylpiperazin-1-yl)benzoate (334.7 mg, 0.63 mmol, 1.10 equiv). The resulting solution was stirred for 2 h at 110 degree. The reaction mixture was cooled to room temperature. The solids were filtered out. The resulting mixture was concentrated under vacuum. The residue was applied onto a silica gel column with ethyl acetate/petroleum ether (0:1-1:3). This resulted in 200 mg (46%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-13-oxa-2,4,10-triazatricyclo[7.4.0.0\hat{ }[3,7]]trideca-1,3(7),5,8-tetraen-10-yl)benzoate as yellow oil. LC-MS: (ES, m/z): M+1=756, R,T=1.252 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Into a 50-mL round-bottom flask, was placed a solution of methyl 4-(4-[[2-(4-chlorophenyl)-4,4-dimethylcyclohex-1-en-1-yl]methyl]piperazin-1-yl)-2-(4-[[2-(trimethylsilyl) ethoxy]methyl]-13-oxa-2,4,10-triazatricyclo[7.4.0.0\hat{ }[3,7]]trideca-1,3(7),5,8-tetraen-10-yl)benzoate (200 mg, 0.26 mmol, 1.00 equiv) in tetrahydrofuran (20 mL), TBAF (3 mg, 0.01 mmol), ethane-1,2-diamine (3 mL). The resulting solution was stirred for 24 h at 60 degree. The reaction mixture was cooled to room temperature. The reaction was then quenched by the addition of 30 mL of water. The resulting solution was extracted with 2×30 mL of ethyl acetate and the organic layers combined. The resulting mixture was washed with 2×30 mL of brine. The mixture was dried over anhydrous sodium sulfate and concentrated under vacuum. The residue was applied onto a silica gel column with ethyl acetate/petroleum ether (0:1-1:2). This resulted in 90 mg (54%) of methyl 4-(4-[[2-(4-chlorophenyl)-4,4-dimethylcyclohex-1-en-1-yl]methyl]piperazin-1-yl)-2-[13-oxa-2,4,10-triazatricyclo[7.4.0.0\hat{ }[3,7]]trideca-1,3(7),5,8-tetraen-10-yl]benzoate as a yellow solid. LC-MS(ES, m/z): M+1=626, R,T=1.052 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Into a 8-mL round-bottom flask, was placed a solution of methyl 4-(4-[[2-(4-chlorophenyl)-4,4-dimethylcyclohex-1-en-1-yl]methyl]piperazin-1-yl)-2-[13-oxa-2,4,10-triazatricyclo[7.4.0.0\hat{ }[3,7]]trideca-1,3(7),5,8-tetraen-10-yl]benzoate (90 mg, 0.14 mmol, 1.00 equiv) in tetrahydrofuran/MeOH/H₂O (2/2/2 mL), sodium hydroxide (23 mg, 0.57 mmol, 4.00 equiv). The resulting solution was stirred for overnight at 60 degree. The reaction mixture was cooled to room temperature. The reaction was then quenched by the addition of 5 mL of water. The pH value of the solution was adjusted to 6 with hydrogen chloride (1 mol/L). The resulting solution was extracted with 2×10 mL of ethyl acetate and the organic layers combined. The resulting mixture was washed with 3×10 mL of brine. The mixture was dried over anhydrous sodium sulfate and concentrated under vacuum. The residue was applied onto a silica gel column with ethyl acetate/petroleum ether (0:1-1:1). This resulted in 70 mg (80%) of 4-(4-[[2-(4-chlorophenyl)-4,4-dimethylcyclohex-1-en-1-yl]methyl]piperazin-1-yl)-2-[13-oxa-2,4,10-triazatricyclo[7.4.0.0\hat{ }[3,7]]trideca-1,3(7),5,8-tetraen-10-yl]benzoic acid as a yellow solid.

LC-MS(ES, m/z): M+1=612, R,T=1.005 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Into a 8-mL round-bottom flask, was placed a solution of 4-(4-[[2-(4-chlorophenyl)-4,4-dimethylcyclohex-1-en-1-yl]methyl]piperazin-1-yl)-2-[13-oxa-2,4,10-triazatricyclo[7.4.0.0\hat{ }[3,7]]trideca-1,3(7),5,8-tetraen-10-yl]benzoic acid (35 mg, 0.06 mmol, 1.00 equiv) in dichloromethane (5 mL), 4-dimethylaminopyridine (27.8 mg, 0.23 mmol, 4.00 equiv), 3-nitro-4-[(oxan-4-ylmethyl)amino]benzene-1-sulfonamide (21.7 mg, 0.07 mmol, 1.20 equiv), EDCI (22 mg, 0.11 mmol, 2.00 equiv). The resulting solution was stirred for overnight at room temperature. The resulting mixture was concentrated under vacuum. The crude product was purified by Prep-HPLC with the following conditions (Waters-2767): Column, X-bridge RP18, 5 um, 19*100 mm; mobile phase, 0.03% ammonia in water (0.03% NH4HCO3 & NH4OH) and CH3CN (32% CH3CN up to 52% in 6 min); Detector, UV 254 nm. This resulted in 28.3 mg (54%) of 4-(4-[[2-(4-chlorophenyl)-4,4-dimethylcyclohex-1-en-1-yl]methyl]piperazin-1-yl)-N-([3-nitro-4-[(oxan-4-ylmethyl)amino]benzene]sulfonyl)-2-[13-oxa-2,4,10-triazatricyclo[7.4.0.0\hat{ }[3,7]]trideca-1,3(7),5,8-tetraen-10-yl]benzamide as a yellow solid. LC-MS-: (ES, m/z): (ES, m/z): M+1=909, R.T=1.52 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient. H-NMR: (CDCl₃, 300 MHz) δ: 8.70 (s, 1H), 8.46 (m, 2H), 8.10-8.06 (m, 1 H), 7.89-7.60 (m, 1H), 7.10 (s, 1H), 6.94-6.71 (m, 5H), 6.49 (s, 1H), 6.16 (s, 1H), 4.70-4.65 (m, 2H), 4.00-4.10 (m, 2H), 3.67-3.19 (m, 7H), 3.20-3.00 (m, 4H), 2.78 (s, 1H), 2.58-2.52 (m, 2H), 2.27-2.17 (m, 3H), 2.05-1.98 (m, 4H), 1.74-1.70 (m, 3H), 1.55-1.40 (m, 3H), 0.98 (s, 6H).The measurements of the NMR spectra were done with BrukerAvanceIII HD 300 MHz with a probe head of BBOF.

### Compound 2-2: Preparation of 4-(4-((4′-chloro-5,5-dimethyl-3,4,5,6-tetrahydro-[1,1′-biphenyl]-2-yl)methyl)piperazin-1-yl)-2-(2,3-dihydropyrrolo[3′,2′:5,6]pyrido[2,3-b][1,4]oxazin-1(6H)-yl)-N-((4-(((4-fluorotetrahydro-2H-pyran-4-yl)methyl)amino)-3-nitrophenyl)sulfonyl)benzamide

Into a 8-mL round-bottom flask, was placed a solution of 4-(4-[[2-(4-chlorophenyl)-4,4-dimethylcyclohex-1-en-1-yl]methyl]piperazin-1-yl)-2-[13-oxa-2,4,10-triazatricyclo[7.4.0.0\hat{ }[3,7]]trideca-1,3(7),5,8-tetraen-10-yl]benzoic acid (35 mg, 0.06 mmol, 1.00 equiv) in dichloromethane (5 mL), 4-dimethylaminopyridine (27.8 mg, 0.23 mmol, 4.00 equiv), EDCI (22 mg, 0.11 mmol, 2.00 equiv), 4-[(4-fluorooxan-4-yl)methyl]amino-3-nitrobenzene-1-sulfonamide (22.7 mg, 0.07 mmol, 1.20 equiv). The resulting solution was stirred for overnight at room temperature. The resulting mixture was concentrated under vacuum. The crude product was purified by Prep-HPLC with the following conditions (Waters-2767): Column, X-bridge RP18, Sum, 19*100 mm; mobile phase, 0.03% ammonia in water (0.03% NH₄HCO₃ & NH₄OH) and CH₃CN (32% CH₃CN up to 52% in 6 min); Detector, UV 254 nm. This resulted in 26.2 mg (50%) of 4-(4-[[2-(4-chlorophenyl)-4,4-dimethylcyclohex-1-en-1-yl]methyl]piperazin-1-yl)-N-[(4-[[(4-fluorooxan-4-yl)methyl]amino]-3-nitrobenzene)sulfonyl]-2-[13-oxa-2,4,10-triazatricyclo[7.4.0.0\hat{ }[3,7]]trideca-1,3(7),5,8-tetraen-10-yl]benzamide as a yellow solid. LC-MS: (ES, m/z): (ES, m/z): M+1=927, R,T=1.27 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR: (CDCl₃, 300 MHz) δ: 12.38 (bs, 1H), 8.69 (d, J=2.1 Hz, 1H), 8.58 (t, J=6.3 Hz, 1H), 8.44 (s, 1H), 8.07 (d, J=9.0 Hz, 1H), 7.90-7.87 (m, 1H), 7.24-7.22 (m, 2H), 7.08 (s, 1H), 6.94 (m, 2H), 6.85 (s, 1H), 6.80-6.77 (m, 2H), 6.49 (s, 1H), 6.14 (s, 1H), 4.74-4.67 (m, 2H), 3.91-3.80 (m, 2H), 3.80-3.44 (m, 6H), 3.17 (m, 4H), 2.77 (s, 1H), 2.22-2.10 (m, 6H), 2.00 (s, 2H), 1.98-1.75 (m, 3H), 1.80-1.60 (m, 2H), 1.55-1.40 (m, 2H), 0.94 (s, 6H). The measurements of the NMR spectra were done with BrukerAvanceIII HD 300 MHz with a probe head of BBOF.

### Compound 2-3: Preparation of 4-(4-((4′-chloro-5,5-dimethyl-3,4,5,6-tetrahydro-[1,1′-biphenyl]-2-yl)methyl)piperazin-1-yl)-2-(2,3-dihydropyrrolo[3′,2′:5,6]pyrido[2,3-b][1,4]oxazin-1(6H)-yl-2,2,3,3-d4)-N-((4-(((4-fluorotetrahydro-2H-pyran-4-yl)methyl)amino)-3-nitrophenyl)sulfonyl)benzamide

Synthesis of 4-[[2-(trimethylsilyl)ethoxy]methyl](12,12-2H2)-13-oxa-2,4,10-triazatricyclo[7.4.0.0\hat{ }[3,7]]trideca-1(9),2,5,7-tetraen-11-one: Into a 8-mL vial, was placed 4-[[2-(trimethylsilyl)ethoxy]methyl]-13-oxa-2,4,10-triazatricyclo[7.4.0.0\hat{ }[3,7]]trideca-1(9),2,5,7-tetraen-11-one (90 mg, 0.28 mmol, 1 equiv), D20 (1 mL), MeOD (1 mL), Na₂CO₃ (89.6 mg, 0.85 mmol, 3.00 equiv). The resulting solution was stirred for 48 h at 60° C. in an oil bath. The resulting solution was extracted with 3×3 mL of dichloromethane. The organic layer was dried over anhydrous sodium sulfate and concentrated under vacuum. This resulted in 60 mg (66%) of 4-[[2-(trimethylsilyl)ethoxy]methyl](12,12-2H2)-13-oxa-2,4,10-triazatricyclo[7.4.0.0\hat{ }[3,7]]trideca-1(9),2,5,7-tetraen-11-one as a yellow solid.LC-MS-PH-PHNW-4-34-1: (ES, m/z): M+1=322,The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-34-2: (d-DMSO, 300 ppm): 7.44-7.41 (m, 2H), 6.43-6.42 (d, J=6 Hz, 1H), 5.46-5.41 (m, 2H), 3.51-3.46 (m, 2H), 1.24 (s, 1H), 0.86-0.79 (m, 4H), -0.04--0.05 (m, 9H).

Synthesis of 4-[[2-(trimethylsilyl)ethoxy]methyl](11,11,12,12-2H4)-13-oxa-2,4,10-triazatricyclo[7.4.0.0\hat{ }[3,7]]trideca-1(9),2,5,7-tetraene: Into a 8-mL vial, was placed 4-[[2-(trimethylsilyl)ethoxy]methyl](12,12-2H2)-13-oxa-2,4,10-triazatricyclo[7.4.0.0\hat{ }[3,7]]trideca-1(9),2,5,7-tetraen-11-one (60 mg, 0.19 mmol, 1 equiv), THE (3 mL). This was followed by the addition of LiAlD₄ (31.3 mg, 0.75 mmol, 3.99 equiv) in portions at 0° C. The resulting solution was stirred for overnight at room temperature. The reaction was then quenched by the addition of 1 mL of D₂O. The resulting solution was extracted with 3×5 mL of ethyl acetate. The organic layer was dried over anhydrous sodium sulfate and concentrated under vacuum. This resulted in 25 mg (43.28%) of 4-[[2-(trimethylsilyl)ethoxy]methyl](11,11,12,12-2H4)-13-oxa-2,4,10-triazatricyclo[7.4.0.0\hat{ }[3,7]]trideca-1(9),2,5,7-tetraene as a yellow solid. LC-MS-PH-PHNW-4-34-2: (ES, m/z): M+1=931. H-NMR-PH-PHNW-4-34-2: (CDCl₃,300 ppm): 7.35 (s, 1H), 7.17-7.15 (d, J=6 Hz, 1H), 6.35-6.34 (d, J=3 Hz, 1H), 5.56-5.53 (m, 2H), 3.58-3.52 (m, 2H), 2.08 (s, 1H), 1.28 (s, 1H), 0.93-0.88 (m, 3H), -0.04--0.05 (m, 9H).

### Synthesis of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl](11,11,12,12-2H4)-13-oxa-2,4,10-triazatricyclo[7.4.0.0\hat{ }[3,7]]trideca-1(9),2,5,7-tetraen-10-yl)benzoate: Into a 8-mL vial, was placed 4-[[2-(trimethylsilyl)ethoxy]methyl](11,11,12,12-2H4)-13-oxa-2,4,10-triazatricyclo[7.4.0.0\hat{ }[3,7]]trideca-1(9),2,5,7-tetraene (25 mg, 0.08 mmol, 1 equiv), methyl 2-bromo-4-(4-[[2-(4-chlorophenyl)-4,4-dimethylcyclohex-1-en-1-yl]methyl]piperazin-1-yl)benzoate (55.9 mg, 0.11 mmol, 1.3 equiv), Dioxane (5 mL), Cs₂CO₃ (52.6 mg, 0.16 mmol, 2 equiv), Chloro[9,9-dimethyl-4,5-bis(diphenylphosphino)xanthene][2-amino-1,1-biphenyl-2-yl]palladium(II) (5 mg). The resulting solution was stirred for 2 h at 100° C. in an oil bath. After the reaction completed, the crude solution is concentrated and the residue was applied onto a silica gel column with ethyl acetate/petroleum ether (0:1-1:1). This resulted in 20 mg (32.56%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl](11,11,12,12-2H4)-13-oxa-2,4,10-triazatricyclo[7.4.0.0\hat{ }[3,7]]trideca-1(9),2,5,7-tetraen-10-yl)benzoate as a yellow solid. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min

Synthesis of methyl 4-(4-[[2-(4-chlorophenyl)-4,4-dimethylcyclohex-1-en-1-yl]methyl]piperazin-1-yl)-2-[(11,11,12,12-2H4)-13-oxa-2,4,10-triazatricyclo[7.4.0.0\hat{ }[3,7]]trideca-1(9),2,5,7-tetraen-10-yl]benzoate. Into a 8-mL vial, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl](11,11,12,12-2H4)-13-oxa-2,4,10-triazatricyclo[7.4.0.0\hat{ }[3,7]]trideca-1(9), (20 mg, 0.03 mmol, 1 equiv), THE (2 mL), TBAF (100 mg, 0.38 mmol, 14.54 equiv), ethane-1,2-diamine (1 mL, 0.02 mmol, 0.63 equiv). The resulting solution was stirred for overnight at 70° C. The resulting mixture was concentrated under vacuum. The residue was applied onto a silica gel column with ethyl acetate/petroleum ether (0:1-1:1). This resulted in 12 mg (72.40%) of methyl 4-(4-[[2-(4-chlorophenyl)-4,4-dimethylcyclohex-1-en-1-yl]methyl]piperazin-1-yl)-2-[(11,11,12,12-2H4)-13-oxa-2,4,10-triazatricyclo[7.4.0.0\hat{ }[3,7]]trideca-1(9),2,5,7-tetraen-10-yl]benzoate as a yellow solid. LC-MS-PH-PHNW-4-34-4: (ES, m/z): M+1=630. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of 4-(4-[[2-(4-chlorophenyl)-4,4-dimethylcyclohex-1-en-1-yl]methyl]piperazin-1-yl)-2-[(11,11,12,12-2H4)-13-oxa-2,4,10-triazatricyclo[7.4.0.0\hat{ }[3,7]]trideca-1(9),2,5,7-tetraen-10-yl]benzoic acid: Into a 8-mL vial, was placed methyl 4-(4-[[2-(4-chlorophenyl)-4,4-dimethylcyclohex-1-en-1-yl]methyl]piperazin-1-yl)-2-[(11,11,12,12-2H4)-13-oxa-2,4,10-triazatricyclo[7.4.0.0\hat{ }[3,7]]trideca-1(9),2,5,7-tetraen-10-yl]benzoate (12 mg, 0.02 mmol, 1 equiv), MeOH (1 mL), H₂O (1 mL), THF (1 mL), NaOH (3.0 mg, 0.08 mmol, 3.94 equiv). The resulting solution was stirred for overnight at 60° C. in an oil bath. The pH value of the solution was adjusted to 5 with HCl (1 mol/L). The reaction mixture was concentrated under vacuum. The residue was applied on a silica gel column and eluted with PE/EA(1:0-2:3). This resulted in 9 mg (76.71%) of 4-(4-[[2-(4-chlorophenyl)-4,4-dimethylcyclohex-1-en-1-yl]methyl]piperazin-1-yl)-2-[(11,11,12,12-2H4)-13-oxa-2,4,10-triazatricyclo[7.4.0.0\hat{ }[3,7]]trideca-1(9),2,5,7-tetraen-10-yl]benzoic acid as an off-white solid. LC-MS-PH-PHNW-4-34-5: (ES, m/z): M+1=616. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(11,11,12,12-2H4)-13-oxa-2,4,10-triazatricyclo[7.4.0.0.0\hat{ }[3,7]]trideca-1(9),2,5,7-tetraen-10-yl]benzamide: Into a 8-mL vial, was placed 4-(4-[[2-(4-chlorophenyl)-4,4-dimethylcyclohex-1-en-1-yl]methyl]piperazin-1-yl)-2-[(11,11,12,12-2H4)-13-oxa-2,4,10-triazatricyclo[7.4.0.0\hat{ }[3,7]]trideca-1,3(7),5,8-tetraen-10-yl]benzoic acid (9 mg, 0.01 mmol, 1 equiv), 4-[[(4-fluorooxan-4-yl)methyl]amino]-3-nitrobenzene-1-sulfonamide (6.3 mg, 0.02 mmol, 1.3 equiv), DCM (2 mL), DMAP (7.1 mg, 0.06 mmol, 3.98 equiv), EDCI (5.6 mg, 0.03 mmol, 2 equiv). The resulting solution was stirred for overnight at room temperature.

The resulting mixture was concentrated under vacuum. The residue was applied onto a silica gel column with dichloromethane/methanol (10:1). This resulted in 6 mg (44.10%) of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(11,11,12,12-2H4)-13-oxa-2,4,10-triazatricyclo[7.4.0.0.0\hat{ }[3,7]]trideca-1(9),2,5,7-tetraen-10-yl]benzamide as a yellow solid. LC-MS-PH-PHNW-4-34-0: (ES, m/z): M+1=931. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-34-0: (d-DMSO, 300 ppm): 8.57 (s, 1H), 8.37 (s, 1H), 7.58-7.55 (m, 1H), 7.37-7.35 (m, 3H), 7.08-7.05 (m, 3H), 6.87-6.76 (m, 3H), 3.76-3.73 (m, 6H), 3.57-3.53 (m, 6H), 3.33 (m, 3H), 2.76-2.73 (m, 2H), 2.26-2.20 (m, 6H), 1.98 (m, 2H), 1.81-1.76 (m, 5H), 1.41-1.39 (m, 5H), 1.39 (m, 4H), 0.91-0.88 (m, 6H).

### Compound 2-4: Preparation of 4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(2-methyl-2,3-dihydropyrrolo[3′,2′:5,6]pyrido[2,3-b][1,4]oxazin-1(6H)-yl)benzamide

Synthesis of tert-butyl 1-(5-bromo-1-((2-(trimethylsilyl)ethoxy)methyl)-1H-pyrrolo[2,3-b]pyridin-6-yloxy)propan-2-ylcarbamate. Into a 250 mL round-bottom flask was placed tert-butyl 1-hydroxypropan-2-ylcarbamate (2.28 g, 13.05 mmol, 1.50 equiv), DMF (30 mL), NaH (0.87 g, 21.75 mmol, 2.50 equiv) at 0° C. for 10 min. 5-bromo-6-fluoro-1-((2-(trimethylsilyl)ethoxy)methyl)-1H-pyrrolo[2,3-b]pyridine (3.0 g, 8.70 mmol, 1.00 equiv) was added. The resulting solution was stirred overnight at room temperature. The resulting solution was diluted with 200 mL of H₂O. The resulting solution was extracted with 3×300 mL of ethyl acetate and the organic layers combined. The resulting mixture was washed with 1×200 mL of H₂O and 1×200 mL sodium chloride(aq). The resulting mixture was concentrated under vacuum. The residue was applied onto a silica gel column with PE/EA (5:1). This resulted in 2.2 g (50%) of tert-butyl 1-(5-bromo-1-((2-(trimethylsilyl)ethoxy)methyl)-1H-pyrrolo[2,3-b]pyridin-6-yloxy)propan-2-ylcarbamate as a yellow oil. LC-MS-PH-PHNW-4-35-1(ES, m/z): LC-MS (M+1): 502; RT=1.50 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 2.0 minutes; Oven temperature 40° C.; flow:1.5 mL/min.

Synthesis of tert-butyl 2-methyl-6-((2-(trimethylsilyl)ethoxy)methyl)-2,3-dihydropyrrolo[3′,2′:5,6]pyrido[2,3-b][1,4]oxazine-1(6H)-carboxylate. Into a 100 mL round-bottom flask was placed tert-butyl 1-(5-bromo-1-((2-(trimethylsilyl)ethoxy)methyl)-1H-pyrrolo[2,3-b]pyridin-6-yloxy)propan-2-ylcarbamate (2.2 g, 4.39 mmol, 1.00 equiv), dioxane (25 mL), Cs₂CO₃ (4.30 g, 13.17 mmol, 3.00 equiv), X-phosPd 3G (1.04 g, 1.317 mmol, 0.30 equiv). The resulting solution was stirred overnight at 100° C. under N2. The resulting mixture was concentrated under vacuum. The residue was applied onto a silica gel column with PE/EA (3:1).This resulted in 1.26 g (68%) of tert-butyl 2-methyl-6-((2-(trimethylsilyl)ethoxy)methyl)-2,3-dihydropyrrolo[3′,2′:5,6]pyrido[2,3-b][1,4]oxazine-1(6H)-carboxylate as a yellow oil.LC-MS-PH-PHNW-4-35-2(ES, m/z): LC-MS (M+1): 420; RT=1.84 min. The measurements of the retention were done with a reversed phase column (C18). Shimadzu LCMS 2020; 50*3.0 Kinetex 2.6u XB-ODS, 2.6 microm; Eluent A: water (0.05% TFA); Eluent B: Acetonitrile; linear gradient from 5% acetonitrile to 100% acetonitrile in 2.6 minutes; Oven temperature 40° C.; flow:1.0 mL/min.

Synthesis of 2-methyl-6-((2-(trimethylsilyl)ethoxy)methyl)-1,2,3,6-tetrahydropyrrolo [3′,2′:5,6]pyrido[2,3-b][1,4]oxazine: Into a 250 mL round-bottom flask was placedtert-butyl 2-methyl-6-((2-(trimethylsilyl)ethoxy)methyl)-2,3-dihydropyrrolo[3′,2′:5,6]pyrido[2,3-b][1,4]oxazine-1(6H)-carboxylate (1.26 g, 3.00 mmol, 1.00 equiv), DCM (15 mL), ZnBr₂ (10.0 g, 30 mmol, 10.0 equiv). The resulting solution was stirred at room temperature for 3 h. The resulting solution was diluted with 50 mL of NaHCO₃. The resulting solution was extracted with 3×50 mL of DCM and the organic layers combined. The resulting mixture was washed with 1×50 mL of H₂O and 1×50 mL sodium chloride(aq). The resulting mixture was concentrated under vacuum. The residue was applied onto a silica gel column with PE/EA (1:1). This resulted in 800 mg (83%) of 2-methyl-6-((2-(trimethylsilyl)ethoxy)methyl)-1,2,3,6-tetrahydropyrrolo[3′,2′:5,6]pyrido[2,3-b][1,4]oxazine as a yellow oil.LC-MS-PH-PHNW-4-35-3(ES, m/z): LC-MS (M+1): 320; RT=1.54 min. The measurements of the retention were done with a reversed phase column (C18). Shimadzu LCMS 2020; 50*3.0 Kinetex 2.6u XB-ODS, 2.6 microm; Eluent A: water (0.05% TFA); Eluent B: Acetonitrile; linear gradient from 5% acetonitrile to 100% acetonitrile in 2.6 minutes; Oven temperature 40° C.; flow:1.0 mL/min.

### Synthesis of methyl 4-(4-((4′-chloro-5,5-dimethyl-3,4,5,6-tetrahydro-[1,1′-biphenyl]-2-yl)methyl) piperazin-1-yl)-2-(2-methyl-6-((2-(trimethylsilyl)ethoxy)methyl)-2,3-dihydropyrrolo[3′,2′:5,6]pyrido[2,3-b][1,4]oxazin-1(6H)-yl)benzoate: Into a 100 mL round-bottom flask was placed tert-butyl 1-(5-bromo-1-((2-(trimethylsilyl)ethoxy)methyl)-1H-pyrrolo[2,3-b]pyridin-6-yloxy)propan-2-ylcarbamate (300 mg, 0.94 mmol, 1.00 equiv), dioxane (15 mL), Cs₂CO₃ (920 mg, 2.82 mmol, 3.00 equiv), XantphosPd 2G (83 mg, 0.094 mmol, 0.10 equiv), methyl 2-bromo-4-(4-((4′-chloro-5,5-dimethyl-3,4,5,6-tetrahydro-[1,1′-biphenyl]-2-yl)methyl)piperazin-1-yl)benzoate(1.0 g, 1.88 mmol, 2.00equiv). The resulting solution was stirred overnight at 110° C. under N2. The resulting mixture was concentrated under vacuum. The residue was applied onto a silica gel column with PE/EA (5:1).This resulted in 360 mg (50%) of methyl 4-(4-((4′-chloro-5,5-dimethyl-3,4,5,6-tetrahydro-[1,1′-biphenyl]-2-yl)methyl)piperazin-1-yl)-2-(2-methyl-6-((2-(trimethylsilyl)ethoxy)methyl)-2,3-dihydropyrrolo[3′,2′:5,6]pyrido[2,3-b][1,4]oxazin-1(6H)-yl)benzoate as a yellow oil. LC-MS-PH-PHNW-4-35-4(ES, m/z): LC-MS (M+1): 770; RT=1.56 min. The measurements of the retention were done with a reversed phase column (C18). Shimadzu LCMS 2020; 50*3.0 Kinetex 2.6u XB-ODS, 2.6 microm; Eluent A: water (0.05% TFA); Eluent B: Acetonitrile; linear gradient from 5% acetonitrile to 100% acetonitrile in 2.6 minutes; Oven temperature 40° C.; flow:1.0 mL/min

Synthesis of methyl 4-(4-((4′-chloro-5,5-dimethyl-3,4,5,6-tetrahydro-[1,1′-biphenyl]-2-yl)methyl) piperazin-1-yl)-2-(2-methyl-2,3-dihydropyrrolo[3′,2′:5,6]pyrido[2,3-b][1,4]oxazin-1(6H)-yl) benzoate Into a 100 mL round-bottom flask was placedtert-butyl methyl 4-(4-((4′-chloro-5,5-dimethyl-3,4,5,6-tetrahydro-[1,1′-biphenyl]-2-yl)methyl)piperazin-1-yl)-2-(2-methyl-6-((2-(trimethylsilyl)ethoxy)methyl)-2,3-dihydropyrrolo[3′,2′:5,6]pyrido[2,3-b][1,4]oxazin-1(6H)-yl)benzoate (300 mg, 0.39 mmol, 1.00 equiv), THE (10 mL), TBAF (3.0 g), ethane-1,2-diamine(5 mL). The resulting solution was stirred overnight at 60° C. The resulting mixture was concentrated under vacuum. The mixture was adjust PH<7 by 2N HCl. The resulting solution was extracted with 3×200 mL of EA and the organic layers combined. The resulting mixture was washed with 1×10 mL of H₂O and 1×10 mL sodium chloride(aq). The resulting mixture was concentrated under vacuum. This resulted in 120 mg (48%) of methyl 4-(4-((4′-chloro-5,5-dimethyl-3,4,5,6-tetrahydro-[1,1′-biphenyl]-2-yl)methyl)piperazin-1-yl)-2-(2-methyl-2,3-dihydropyrrolo[3′,2′:5,6]pyrido[2,3-b][1,4]oxazin-1(6H)-yl)benzoate as a yellow oil. LC-MS-PH-PHNW-4-35-5(ES, m/z): LC-MS (M+1): 640; RT=2.82 min. The measurements of the retention were done with a reversed phase column (C18). Shimadzu LCMS 2020; 50*3.0 Kinetex 2.6u HPH-C18, 2.6 microm; Eluent A: water (0.05% NH₄HCO₃); Eluent B: Methanol; linear gradient from 10% acetonitrile to 98% acetonitrile in 3.5 minutes; Oven temperature 40° C.; flow:0.8 mL/min.

Synthesis of 4-(4-((4′-chloro-5,5-dimethyl-3,4,5,6-tetrahydro-[1,1′-biphenyl]-2-yl)methyl)piperazin-1-yl)-2-(2-methyl-2,3-dihydropyrrolo[3′,2′:5,6]pyrido[2,3-b][1,4]oxazin-1(6H)-yl)benzoic acid. Into a 50 mL round-bottom flask was methyl 4-(4-((4′-chloro-5,5-dimethyl-3,4,5,6-tetrahydro-[1,1′-biphenyl]-2-yl)methyl)piperazin-1-yl)-2-(2-methyl-2,3-dihydropyrrolo[3′,2′:5,6]pyrido[2,3-b][1,4]oxazin-1(6H)-yl)benzoate (70 mg, 0.11 mmol, 1.00 equiv), MeOH/H₂O (5/5 mL), NaOH (44 mg, 1.10 mmol, 10 equiv).The resulting solution was stirred at 60° C. for 3h. The resulting mixture was concentrated under vacuum. The residue was applied onto a silica gel column with dichloromethane/methanol (10:1). This resulted in 50 mg (73%) of 4-(4-((4′-chloro-5,5-dimethyl-3,4,5,6-tetrahydro-[1,1′-biphenyl]-2-yl)methyl)piperazin-1-yl)-2-(2-methyl-2,3-dihydropyrrolo[3′,2′:5,6]pyrido[2,3-b][1,4]oxazin-1(6H)-yl)benzoic acid as a white solid. LC-MS-PH-PHNW-4-35-6(ES, m/z): LC-MS (M+1): 626; RT=2.45 min. The measurements of the retention were done with a reversed phase column (C18). Shimadzu LCMS 2020; 50*3.0 Kinetex 2.6u HPH-C18, 2.6 microm; Eluent A: water (0.05% NH₄HCO₃); Eluent B: Methanol; linear gradient from 10% acetonitrile to 98% acetonitrile in 3.5 minutes; Oven temperature 40° C.; flow:0.8 mL/min.

Synthesis of 4-(4-((4′-chloro-5,5-dimethyl-3,4,5,6-tetrahydro-[1,1′-biphenyl]-2-yl) methy 1)piperazin-1-yl)-N-((4-(((4-fluorotetrahydro-2H-pyran-4-yl)methyl)amino)-3-nitrophenyl 1)sulfonyl)-2-(2-methyl-2,3-dihydropyrrolo[3′,2′:5,6]pyrido[2,3-b][1,4]oxazin-1(6H)-yl)benzamide: Into a 50-mL 1-necked round-bottom flask was placed 4-(4-((4′-chloro-5,5-dimethyl-3,4,5,6-tetrahydro-[1,1′-biphenyl]-2-yl)methyl)piperazin-1-yl)-2-(2-methyl-2,3-dihydropyrrolo[3′,2′:5,6]pyrido[2,3-b][1,4]oxazin-1(6H)-yl)benzoic acid (40 mg, 0.064 mol, 1.00 equiv), DCM (4 mL), EDCI (49 mg, 0.256 mmol, 4.00 equiv), DMAP (16 mg, 0.128 mmol, 2.00 equiv), 4-(((4-fluorotetrahydro-2H-pyran-4-yl)methyl)amino)-3-nitrobenzenesulfonamide(28 mg, 0.0832 mol, 1.30 equiv). The resulting solution was stirred overnight at 40° C. The resulting mixture was concentrated under vacuum. This resulted in 2.9 mg (70%) of 4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(2-methyl-2,3-dihydropyrrolo[3′,2′:5,6]pyrido[2,3-b][1,4]oxazin-1(6H)-yl)benzamide as a yellow solid.LC-MS-PH-PHNW-4-35-0A(ES, m/z): LC-MS (M+1):941; RT=5.02 min. The measurements of the retention were done with a reversed phase column (C18). Shimadzu LCMS 2020; 50*3.0 Kinetex 2.6u Ascentis Express C18, 2.6 microm; Eluent A: water (0.05% TFA); Eluent B: Methanol; linear gradient from 5% acetonitrile to 95% acetonitrile in 7.0 minutes; Oven temperature 40° C.; flow:1.0 mL/min.

### Compound 2-5: Preparation of (S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide and (R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide

Synthesis of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-11-one: Into a 100-mL round-bottom flask, was placed 5-amino-1-[[2-(trimethylsilyl)ethoxy]methyl]-1H-pyrrolo[2,3-b]pyridin-6-ol (1.5 g, 5.37 mmol, 1 equiv), DMF (50 mL), K₂CO₃ (2.2 g, 16.11 mmol, 3 equiv). This was followed by the addition of 2-chloropropanoyl chloride (1.4 g, 10.74 mmol, 2 equiv) dropwise with stirring at 0° C. The resulting solution was stirred for overnight at room temperature. The reaction was then quenched by the addition of 50 mL of water. The resulting solution was extracted with 2×50 mL of ethyl acetate. The resulting mixture was washed with 2 x50 mL of brine. The mixture was dried over anhydrous sodium sulfate and filtrate and concentrated under vacuum. The residue was applied onto a silica gel column with ethyl acetate/petroleum ether (0:1-1:3). This resulted in 500 mg (27.93%) of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-11-one as a yellow solid. LC-MS-PH-PHNW-4-37-1: (ES, m/z): M+1=334, R,T=1.123 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-37-1: (CDCl₃,300 ppm): 8.34 (s, 1H), 7.63 (d, J=3 Hz, 1H), 7.42-7.28 (m, 1H), 6.46 (d, J=3 Hz, 1H), 5.68 (s, 2H), 4.92-4.85 (m, 1H), 3.63-3.53 (m, 2H), 1.85-1.81 (d, J=12 Hz, 3H), 0.94-0.89 (m, 2H), -0.154 (s, 9H). The measurements of the NMR spectra were done with BrukerAvanceIII HD 300 MHz with a probe head of BBOF.

Synthesis of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene & (12R or S)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene(Assumed) & (12S or R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene(Assumed): Into a 100-mL 3-necked round-bottom flask, was placed 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-11-one (500 mg, 1.50 mmol, 1 equiv), THE (20 mL). This was followed by the addition of LiAlH₄ (113.8 mg, 3.00 mmol, 2 equiv), in portions at 0° C. The resulting solution was stirred for 1 overnight at room temperature. The reaction was then quenched by the addition of 20 mL of water. The solids were filtered out. The resulting solution was extracted with 2×20 mL of ethyl acetate. The resulting mixture was washed with 2 x20 mL of brine. The mixture was dried over anhydrous sodium sulfate and filtrate and concentrated under vacuum. The residue was applied onto a silica gel column with ethyl acetate/petroleum ether (0:1-1:3). This resulted in 450 mg (93%) of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene as a yellow solid. The crude 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (450 mg) was purified by Chiral-Prep-HPLC with the following conditions: (SHIMADZU LC-20AT): Column, CHIRALPAK IC; mobile phase A:n-hexane, Phase B:ethanol; Detector, 220 nm. This resulted in 200 mg (44%) of (12R or S)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene as a yellow solid(Assumed).

This resulted in 200 mg (44%) of (12S or R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene as a yellow solid(Assumed).

LC-MS-PH-PHNW-4-37-2: (ES, m/z): M+1=320, R,T=1.107 min.

The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

H-NMR-PH-PHNW-4-37-2: (CDCl₃,300 ppm): 7.63 (s, 1H), 7.17 (s, 1H), 6.36-6.35 (d, J=3 Hz, 1H), 5.57-5.52 (m, 2H), 4.59-4.55 (m, 1H), 3.62-3.46 (m, 3H), 3.18-3.14 (m, 1H), 1.62-1.44 (m, 3H), 0.93-0.88 (m, 2H), -0.17 (s, 9H).

Synthesis of methyl 4-(4-[[2-(4-chlorophenyl)-4,4-dimethylcyclohex-1-en-1-yl]methyl]piperazin-1-yl)-2-[(12R or S)-12-methyl-4-[[2-(trimethylsilyl)ethoxy]methyl]-13-oxa-2,4,10-triazatricyclo[7.4.0.0\hat{ }[3,7]]trideca-1(9),2,4,10-triazatricyclo[7.4.0.0\hat{ },7]trideca-1(9),2,5,7-tetraen-10-yl]benzoate(Assumed): Into a 8-mL vial, was placed (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (200 mg, 0.63 mmol, 1 equiv), methyl 2-bromo-4-(4-[[2-(4-chlorophenyl)-4,4-dimethylcyclohex-1-en-1-yl]methyl]piperazin-1-yl)benzoate (399.6 mg, 0.75 mmol, 1.2 equiv), Cs₂CO₃ (611.9 mg, 1.88 mmol, 3 equiv), Dioxane (5 mL), Chloro[9,9-dimethyl-4,5-bis(diphenylphosphino)xanthene][2-amino-1,1-biphenyl-2-yl]palladium(II) (120 mg). The resulting solution was stirred for 2 hr at 110° C. in an oil bath. The resulting mixture was concentrated under vacuum. The residue was applied onto a silica gel column with ethyl acetate/petroleum ether (0:1-1:10). This resulted in 250 mg (51.83%) of methyl 4-(4-[[2-(4-chlorophenyl)-4,4-dimethylcyclohex-1-en-1-yl]methyl]piperazin-1-yl)-2-[(12R or S)-12-methyl-4-[[2-(trimethylsilyl)ethoxy]methyl]-13-oxa-2,4,10-triazatricyclo[7.4.0.0\hat{ }[3,7]]trideca-1(9),2,4,10-triazatricyclo[7.4.0.0\hat{ },7]trideca-1(9),2,5,7-tetraen-10-yl]benzoate as a yellow solid(Assumed).

Synthesis of methyl 4-(4-[[2-(4-chlorophenyl)-4,4-dimethylcyclohex-1-en-1-yl]methyl]piperazin-1-yl)-2-[(12R or S)-12-methyl-13-oxa-2,4,10-triazatricyclo[7.4.0.0\hat{ }[3,7]]trideca-1(9),2,5,7-tetraen-10-yl]benzoate(Assumed) Into a 100-mL round-bottom flask, was placed methyl 4-(4-[[2-(4-chlorophenyl)-4,4-dimethylcyclohex-1-en-1-yl]methyl]piperazin-1-yl)-2-[(12R or S)-12-methyl-4-[[2-(trimethylsilyl)ethoxy]methyl]-13-oxa-2,4,10-triazatricyclo[7.4.0.0\hat{ }[3,7]]trideca-1(9),2 (250 mg, 0.32 mmol, 1 equiv), TBAF (3 g, 11.47 mmol, 35.36 equiv), THE (30 mL), ethane-1,2-diamine (2 g, 33.28 mmol, 102.56 equiv). The resulting solution was stirred for 12 h at 70° C. in an oil bath. The reaction was then quenched by the addition of 50 mL of water. The resulting solution was extracted with 2×50 mL of ethyl acetate. The resulting mixture was washed with 3 x50 mL of brine. The mixture was dried over anhydrous sodium sulfate and filtrate and concentrated under vacuum. The residue was applied onto a silica gel column with ethyl acetate/petroleum ether (0:1-1:3). This resulted in 90 mg (43%) of methyl 4-(4-[[2-(4-chlorophenyl)-4,4-dimethylcyclohex-1-en-1-yl]methyl]piperazin-1-yl)-2-[(12R or S)-12-methyl-13-oxa-2,4,10-triazatricyclo[7.4.0.0\hat{ }[3,7]]trideca-1(9),2,5,7-tetraen-10-yl]benzoate as a yellow solid(Assumed).H-NMR-PH-PHNW-4-37-50: (CDCl₃,300 ppm): 8.24 (s, 1H), 7.89-7.86 (d, J=9 Hz, 1H), 7.32-7.24 (m, 6H), 7.14-7.01 (m, 1H), 6.97-6.94 (m, 2H), 6.73-6.65 (m, 2H), 6.18 (s, 1H), 3.67-3.53 (m, 3H), 3.32-3.18 (m, 3H), 2.98-2.80 (m, 1H), 2.30-2.04 (m, 6H), 1.99 (s, 2H), 1.56-1.50 (m, 5H), 0.91-0.88 (m, 6H).

Synthesis of 4-(4-[[2-(4-chlorophenyl)-4,4-dimethylcyclohex-1-en-1-yl]methyl]piperazin-1-yl)-2-[(12R or S)-12-methyl-13-oxa-2,4,10-triazatricyclo[7.4.0.0\hat{ }[3,7]]trideca-1(9),2,5,7-tetraen-10-yl]benzoic acid(Assumed). Into a 8-mL vial, was placed methyl 4-(4-[[2-(4-chlorophenyl)-4,4-dimethylcyclohex-1-en-1-yl]methyl]piperazin-1-yl)-2-[(12R or S)-12-methyl-13-oxa-2,4,10-triazatricyclo[7.4.0.0\hat{ }[3,7]]trideca-1(9),2,5,7-tetraen-10-yl]benzoate (90 mg, 0.14 mmol, 1 equiv), MeOH (1 mL), H₂O (1 mL), THF (1 mL), NaOH (22.5 mg, 0.56 mmol, 4 equiv). The resulting solution was stirred for overnight at 60° C. in an oil bath. The pH value of the solution was adjusted to 5 with HCl (1 mol/L). The resulting mixture was concentrated under vacuum. The residue was applied onto a silica gel column with dichloromethane/methanol (1:0-10:1). This resulted in 80 mg (90%) of 4-(4-[[2-(4-chlorophenyl)-4,4-dimethylcyclohex-1-en-1-yl]methyl]piperazin-1-yl)-2-[(12R or S)-12-methyl-13-oxa-2,4,10-triazatricyclo[7.4.0.0\hat{ }[3,7]]trideca-1(9),2,5,7-tetraen-10-yl]benzoic acid as a yellow solid(Assumed). LC-MS-PH-PHNW-4-37-60: (ES, m/z): M+1=626, R,T=1.035 min. vThe measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(12R or S)-12-methyl-13-oxa-2,4,10-triazatricyclo[7.4.0.0\hat{ }[3,7]]trideca-1(9),2,5,7-tetraen-10-yl]benzamide(Assumed): Into a 8-mL vial, was placed 4-(4-[[2-(4-chlorophenyl)-4,4-dimethylcyclohex-1-en-1-yl]methyl]piperazin-1-yl)-2-[(12R or S)-12-methyl-13-oxa-2,4,10-triazatricyclo[7.4.0.0\hat{ }[3,7]]trideca-1(9),2,5,7-tetraen-10-yl]benzoic acid (90 mg, 0.14 mmol, 1 equiv), 4-[[(4-fluorooxan-4-yl)methyl]amino]-3-nitrobenzene-1-sulfonamide (57.5 mg, 0.17 mmol, 1.2 equiv), DCM (5 mL), DMAP (70.2 mg, 0.57 mmol, 4 equiv), EDCI (55.1 mg, 0.29 mmol, 2.00 equiv). The resulting solution was stirred for overnight at room temperature. The resulting mixture was concentrated. The crude product was purified by Flash-Prep-HPLC with the following conditions (IntelFlash-1): Column, C18 silica gel; mobile phase, Water(0.I % FA) and ACN (48.0% ACN up to 53.0% in 7 min, hold 95.0% in 1 min, down to 48.0% in 1 min, hold 48.0% in 1 min) within 5 min; Detector, UV 254 nm. This resulted in 28 mg (20%) of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(12R or S)-12-methyl-13-oxa-2,4,10-triazatricyclo[7.4.0.0\hat{ }[3,7]]trideca-1(9),2,5,7-tetraen-10-yl]benzamide as a yellow solid(Assumed). 19.8 mg product was submitted(Assumed). LC-MS-PH-PHNW-4-37-0: (ES, m/z): M+1=941, R,T=3.04 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-37-0: (d-DMSO, 300 ppm): 8.59 (s, 1H), 8.54 (s, 1H), 7.55-7.53 (m, 1H), 7.36-7.29 (d, J=6 Hz, 3H), 7.12-7.06 (m, 3H), 6.80-6.72 (m, 3H), 5.95 (m, 1H), 4.53-4.51 (m, 1H), 3.78-3.43 (m, 7H), 3.21-3.00 (m, 5H), 2.22-2.17 (m, 5H), 1.96-1.75 (m, 6H), 1.58-1.56 (m, 5H), 0.93-0.88 (m, 6H).

Synthesis of methyl 4-(4-[[2-(4-chlorophenyl)-4,4-dimethylcyclohex-1-en-1-yl]methyl]piperazin-1-yl)-2-[(12S or R)-12-methyl-4-[[2-(trimethylsilyl)ethoxy]methyl]-13-oxa-2,4,10-triazatricyclo[7.4.0.0\hat{ }[3,7]]trideca-1(9),2,2,5,7-tetraen-10-yl]benzoate(Assumed): Into a 8-mL vial, was placed (12S or R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (200 mg, 0.63 mmol, 1 equiv), methyl 2-bromo-4-(4-[[2-(4-chlorophenyl)-4,4-dimethylcyclohex-1-en-1-yl]methyl]piperazin-1-yl)benzoate (399.6 mg, 0.75 mmol, 1.2 equiv), Cs₂CO₃ (611.9 mg, 1.88 mmol, 3 equiv), Dioxane (5 mL), Chloro[9,9-dimethyl-4,5-bis(diphenylphosphino)xanthene][2-amino-1,1-biphenyl-2-yl]palladium(II) (120 mg). The resulting solution was stirred for 2 h at 110° C. in an oil bath. The resulting mixture was concentrated under vacuum. The residue was applied onto a silica gel column with ethyl acetate/petroleum ether (0:1-1:10). This resulted in 250 mg (51%) of methyl 4-(4-[[2-(4-chlorophenyl)-4,4-dimethylcyclohex-1-en-1-yl]methyl]piperazin-1-yl)-2-[(12S or R)-12-methyl-4-[[2-(trimethylsilyl)ethoxy]methyl]-13-oxa-2,4,10-triazatricyclo[7.4.0.0\hat{ }[3,7]]trideca-1(9),2,2,5,7-tetraen-10-yl]benzoate as a yellow solid(Assumed).

Synthesis of methyl 4-(4-[[2-(4-chlorophenyl)-4,4-dimethylcyclohex-1-en-1-yl]methyl]piperazin-1-yl)-2-[(12S or R)-12-methyl-13-oxa-2,4,10-triazatricyclo[7.4.0.0\hat{ }[3,7]]trideca-1(9),2,5,7-tetraen-10-yl]benzoate (Assumed): into a 100-mL round-bottom flask, was placed methyl 4-(4-[[2-(4-chlorophenyl)-4,4-dimethylcyclohex-1-en-1-yl]methyl]piperazin-1-yl)-2-[(12S or R)-12-methyl-4-[[2-(trimethylsilyl)ethoxy]methyl]-13-oxa-2,4,10-triazatricyclo[7.4.0.0\hat{ }[3,7]]trideca-1(9),2 (250 mg, 0.32 mmol, 1 equiv), TBAF (3 g, 11.47 mmol, 35.36 equiv), THE (30 mL), ethane-1,2-diamine (2 g, 33.28 mmol, 102.56 equiv). The resulting solution was stirred for 12 h at 70° C. in an oil bath. The reaction was then quenched by the addition of 50 mL of water. The resulting solution was extracted with 2×50 mL of ethyl acetate. The resulting mixture was washed with 3 x50 mL of brine. The mixture was dried over anhydrous sodium sulfate and filtrate and concentrated under vacuum. The residue was applied onto a silica gel column with ethyl acetate/petroleum ether (0:1-1:3). This resulted in 90 mg (43%) of methyl 4-(4-[[2-(4-chlorophenyl)-4,4-dimethylcyclohex-1-en-1-yl]methyl]piperazin-1-yl)-2-[(12S or R)-12-methyl-13-oxa-2,4,10-triazatricyclo[7.4.0.0\hat{ }[3,7]]trideca-1(9),2,5,7-tetraen-10-yl]benzoate as a yellow solid(Assumed).

LC-MS-PH-PHNW-4-38-50: (ES, m/z): M+1=640, R,T=1.424 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-38-50: (CDCl₃, 300 ppm): 8.24 (s, 1H), 7.89-7.86 (d, J=9 Hz, 1H), 7.32-7.24 (m, 6H), 7.14-7.01 (m, 1H), 6.97-6.94 (m, 2H), 6.73-6.65 (m, 2H), 6.18 (s, 1H), 3.67-3.53 (m, 3H), 3.32-3.18 (m, 3H), 2.98-2.80 (m, 1H), 2.30-2.04 (m, 6H), 1.99 (s, 2H), 1.56-1.50 (m, 5H), 0.91-0.88 (m, 6H).

Synthesis of 4-(4-[[2-(4-chlorophenyl)-4,4-dimethylcyclohex-1-en-1-yl]methyl]piperazin-1-yl)-2-[(12S or R)-12-methyl-13-oxa-2,4,10-triazatricyclo[7.4.0.0\hat{ }[3,7]]trideca-1(9),2,5,7-tetraen-10-yl]benzoic acid (Assumed): Into a 8-mL vial, was placed methyl 4-(4-[[2-(4-chlorophenyl)-4,4-dimethylcyclohex-1-en-1-yl]methyl]piperazin-1-yl)-2-[(12S or R)-12-methyl-13-oxa-2,4,10-triazatricyclo[7.4.0.0\hat{ }[3,7]]trideca-1(9),2,5,7-tetraen-10-yl]benzoate (90 mg, 0.14 mmol, 1 equiv), MeOH (1 mL), H₂O (1 mL), THF (1 mL), NaOH (22.5 mg, 0.56 mmol, 4.00 equiv).

The resulting solution was stirred for overnight at 60° C. in an oil bath. The pH value of the solution was adjusted to 5 with HCl (1 mol/L). The resulting mixture was concentrated under vacuum. The residue was applied onto a silica gel column with chloroform/methanol (1:0-10:1). This resulted in 80 mg (90%) of 4-(4-[[2-(4-chlorophenyl)-4,4-dimethylcyclohex-1-en-1-yl]methyl]piperazin-1-yl)-2-[(12S or R)-12-methyl-13-oxa-2,4,10-triazatricyclo[7.4.0.0\hat{ }[3,7]]trideca-1(9),2,5,7-tetraen-10-yl]benzoic acid as a solid(Assumed): LC-MS-PH-PHNW-4-38-60: (ES, m/z): M+1=626, R,T=1.039 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(12S or R)-12-methyl-13-oxa-2,4,10-triazatricyclo[7.4.0.0\hat{ }[3,7]]trideca- 1(9),2,5,7-tetraen-10-yl]benzamide(Assumed): Into a 8-mL vial, was placed 4-(4-[[2-(4-chlorophenyl)-4,4-dimethylcyclohex-1-en-1-yl]methyl]piperazin-1-yl)-2-[(12S or R)-12-methyl-13-oxa-2,4,10-triazatricyclo[7.4.0.0\hat{ }[3,7]]trideca-1(9),2,5,7-tetraen-10-yl]benzoic acid (90 mg, 0.14 mmol, 1 equiv), 4-[[(4-fluorooxan-4-yl)methyl]amino]-3-nitrobenzene-1-sulfonamide (57.5 mg, 0.17 mmol, 1.2 equiv), DCM (5 mL), DMAP (70.2 mg, 0.57 mmol, 4 equiv), EDCI (55.1 mg, 0.29 mmol, 2 equiv). The resulting solution was stirred for overnight at room temperature. The resulting mixture was concentrated. The crude product was purified by Flash-Prep-HPLC with the following conditions (IntelFlash-1): Column, C18 silica gel; mobile phase, Water (0.1% FA) and ACN (48.0% ACN up to 53.0% in 7 min, hold 95.0% in 1 min, down to 48.0% in 1 min, hold 48.0% in 1 min) within 5 min; Detector, UV 254 nm. This resulted in 26 mg (19%) of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(12S or R)-12-methyl-13-oxa-2,4,10-triazatricyclo[7.4.0.0\hat{ }[3,7]]trideca- 1(9),2,5,7-tetraen-10-yl]benzamide as a yellow solid. 19.6 mg product was submitted(Assumed). LC-MS-PH-PHNW-4-38-0: (ES, m/z): M+1=941, R,T=3.036 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-38-0: (d-DMSO, 300 ppm): 8.56 (s, 1H), 8.36 (s, 1H), 7.62-7.54 (m, 1H), 7.37-7.30 (m, 3H), 7.14-7.04 (m, 3H), 6.98-6.92 (m, 3H), 5.94 (m, 1H), 4.53 (m, 1H), 3.79-3.71 (m, 4H), 3.65-3.54 (m, 3H), 3.44 (m, 4H), 2.78-2.73 (m, 2H), 2.23-2.18 (m, 6H), 1.97-1.91 (m, 2H), 1.87-1.81 (m, 4H), 1.50-1.20 (m, 5H), 0.91-0.88 (m, 6H).

### Compound 2-6: Preparation of 4-(4-((4′-chloro-5,5-dimethyl-3,4,5,6-tetrahydro-[1,1′-biphenyl]-2-yl)methyl)piperazin-1-yl)-2-(3,3-dimethyl-2,3-dihydropyrrolo[3′,2′:5,6]pyrido[2,3-b][1,4]oxazin-1(6H)-yl)-N-((4-(((4-fluorotetrahydro-2H-pyran-4-yl)methyl)amino)-3-nitrophenyl)sulfonyl)benzamide

Synthesis of 12,12-dimethyl-4-[[2-(trimethylsilyl)ethoxy]methyl]-13-oxa-2,4,10-triazatricyclo[7.4.0.0\hat{ }[3,7]]trideca-1,3(7),5,8-tetraen-11-one: Into a 100-mL round-bottom flask, was placed 5-amino-1-[[2-(trimethylsilyl)ethoxy]methyl]-1H-pyrrolo[2,3-b]pyridin-6-ol (1 g, 3.58 mmol, 1 equiv), CH₃CN (20 mL), methyl 2-bromo-2-methylpropanoate (647.9 mg, 3.58 mmol, 1.0 equiv), Cs₂CO₃ (1.7 g, 5.37 mmol, 1.5 equiv). The resulting solution was stirred for 2 h at 80° C. in an oil bath. The resulting mixture was concentrated under vacuum. The residue was applied onto a silica gel column with ethyl acetate/petroleum ether (1:3). This resulted in 270 mg (21%) of 12,12-dimethyl-4-[[2-(trimethylsilyl)ethoxy]methyl]-13-oxa-2,4,10-triazatricyclo[7.4.0.0\hat{ }[3,7]]trideca-1,3(7),5,8-tetraen-11-one as a light yellow solid. LC-MS-PH-PHNW-4-40-2: (ES, m/z): M+1=348, R,T=1.164 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of 12,12-dimethyl-4-[[2-(trimethylsilyl)ethoxy]methyl]-13-oxa-2,4,10-triazatricyclo[7.4.0.0\hat{ }[3,7]]trideca-1,3(7),5,8-tetraene: Into a 8-mL vial, was placed 12,12-dimethyl-4-[[2-(trimethylsilyl)ethoxy]methyl]-13-oxa-2,4,10-triazatricyclo[7.4.0.0\hat{ }[3,7]]trideca-1,3(7),5,8-tetraen-11-one (250 mg, 0.72 mmol, 1 equiv), THE (3 mL). This was followed by the addition of LiAlH₄ (54.6 mg, 1.44 mmol, 2 equiv), in portions at 0° C. The resulting solution was stirred for overnight at room temperature. The reaction was then quenched by the addition of 5 mL of water. The solids were filtered out. The resulting solution was extracted with 2×10 mL of ethyl acetate. The organic layer was dried over anhydrous sodium sulfate and concentrated under vacuum. The residue was applied onto a silica gel column with ethyl acetate/petroleum ether (0:1-1:5). This resulted in 140 mg (58%) of 12,12-dimethyl-4-[[2-(trimethylsilyl)ethoxy]methyl]-13-oxa-2,4,10-triazatricyclo[7.4.0.0\hat{ }[3,7]]trideca-1,3(7),5,8-tetraene as a yellow solid.

Synthesis of 4-(4-[[2-(4-chlorophenyl)-4,4-dimethylcyclohex-1-en-1-yl]methyl]piperazin-1-yl)-2-(12,12-dimethyl-4-[[2-(trimethylsilyl)ethoxy]methyl]-13-oxa-2,4,10-triazatricyclo[7.4.0.0\hat{ }[3,7]]trideca-1,3(7), 5,8-tetraen-10-yl)benzoate: Into a 8-mL vial, was placed 12,12-dimethyl-4-[[2-(trimethylsilyl)ethoxy]methyl]-13-oxa-2,4,10-triazatricyclo[7.4.0.0\hat{ }[3,7]]trideca-1,3(7),5,8-tetraene (140 mg, 0.42 mmol, 1 equiv), methyl 2-bromo-4-(4-[[2-(4-chlorophenyl)-4,4-dimethylcyclohex-1-en-1-yl]methyl]piperazin-1-yl)benzoate (267.9 mg, 0.50 mmol, 1.2 equiv), Cs₂CO₃ (410.3 mg, 1.26 mmol, 3 equiv), Dioxane (2 mL), Chloro[9,9-dimethyl-4,5-bis(diphenylphosphino)xanthene][2-amino-1,1-biphenyl-2-yl]palladium(II) (80 mg). The resulting solution was stirred for 2 h at 110° C. in an oil bath. The resulting mixture was concentrated under vacuum. The residue was applied onto a silica gel column with ethyl acetate/petroleum ether (0:1-1:3). This resulted in 130 mg (39%) of methyl 4-(4-[[2-(4-chlorophenyl)-4,4-dimethylcyclohex-1-en-1-yl]methyl]piperazin-1-yl)-2-(12,12-dimethyl-4-[[2-(trimethylsilyl)ethoxy]methyl]-13-oxa-2,4,10-triazatricyclo[7.4.0.0\hat{ }[3,7]]trideca-1,3(7), 5,8-tetraen-10-yl)benzoate as a yellow solid. LC-MS-PH-PHNW-4-40-4: (ES, m/z): M+1=784, R,T=1.331 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of methyl 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1,3(7),5,8-tetraen-10-yl]benzoate: Into a 40-mL vial, was placed methyl 4-(4-[[2-(4-chlorophenyl)-4,4-dimethylcyclohex-1-en-1-yl]methyl]piperazin-1-yl)-2-(12,12-dimethyl-4-[[2-(trimethylsilyl)ethoxy]methyl]-13-oxa-2,4,10-triazatricyclo[7.4.0.0\hat{ }[3,7]]trideca-1,3(7), (130 mg, 0.17 mmol, 1 equiv), TBAF (3 g), THF (10 mL), ethane-1,2-diamine (2 g). The resulting solution was stirred for overnight at 60° C. in an oil bath. The reaction was then quenched by the addition of 10 mL of water. The resulting solution was extracted with 2×10 mL of ethyl acetate. The resulting mixture was washed with 3 x10 mL of brine. The mixture was dried over anhydrous sodium sulfate and filtrate and concentrated under vacuum. The residue was applied onto a silica gel column with ethyl acetate/petroleum ether (0:1-1:3). This resulted in 110 mg (crude) of methyl 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1,3(7),5,8-tetraen-10-yl]benzoate as a yellow solid. LC-MS-PH-PHNW-4-40-5: (ES, m/z): M+1=654, R,T=1.107 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1,3(7),5,8-tetraen-10-yl]benzoic acid: Into a 8-mL vial, was placed methyl 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1,3(7),5,8-tetraen-10-yl]benzoate (80 mg, 0.12 mmol, 1 equiv), MeOH (1 mL), THE (1 mL), H₂O (1 mL), NaOH (19.6 mg, 0.49 mmol, 4 equiv). The resulting solution was stirred for overnight at 60° C. in an oil bath. The pH value of the solution was adjusted to 5 with HCl (1 mol/L). The resulting mixture was concentrated under vacuum. The residue was applied onto a silica gel column with dichloromethane/methanol (1:0-10:1). This resulted in 60 mg (76.64%) of 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1,3(7),5,8-tetraen-10-yl]benzoic acid as a yellow solid. LC-MS-PH-PHNW-4-40-6: (ES, m/z): M+1=640, R,T=1.359 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1(9),2,5,7-tetraen-10-yl]-N-(4-[[(4-fluorooxan-4-yl)methyl]amino]-3-nitrobenzenesulfonyl)benzamide: Into a 8-mL vial, was placed 4-[[(4-fluorooxan-4-yl)methyl]amino]-3-nitrobenzene-1-sulfonamide (37.5 mg, 0.11 mmol, 1.2 equiv), DCM (5 mL), 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1,3(7),5,8-tetraen-10-yl]benzoic acid (60 mg, 0.09 mmol, 1 equiv), EDCI (35.9 mg, 0.19 mmol, 2 equiv), DMAP (45.8 mg, 0.37 mmol, 4 equiv). The resulting solution was stirred for overnight at room temperature. The resulting mixture was concentrated under vacuum. The crude product was purified by Flash-Prep-HPLC with the following conditions (IntelFlash-1): Column, C18 silica gel; mobile phase, Water(0.1% FA) and ACN (48.0% ACN up to 53.0% in 7 min, hold 95.0% in 1 min, down to 48.0% in 1 min within 5; Detector, UV 254 nm. This resulted in 25 mg (27%) of 4-(4-[[2-(4-chlorophenyl)-4,4-dimethylcyclohex-1-en-1-yl]methyl]piperazin-1-yl)-2-[12,12-dimethyl-13-oxa-2,4,10-triazatricyclo[7.4.0.0\hat{ }[3,7]]trideca-1(9),2,5,7-tetraen-10-yl]-N-(4-[[(4-fluorooxan-4-yl)methyl]amino]-3-nitrobenzenesulfonyl)benzamide as a yellow solid. 20.6 mg product was submitted. LC-MS-PH-PHNW-4-40-0: (ES, m/z): M+1=955, R,T=2.655 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-40-0: (d-DMSO, 300 ppm): 8.56 (s, 1H), 8.36 (s, 1H), 7.56-7.49 (m, 2H), 7.36-7.33 (m, 2H), 7.06-7.00 (m, 3H), 6.93-6.90 (m, 1H), 6.88-6.74(M, 2H), 5.99 (m, 1H), 3.78-3.74 (m, 4H), 3.67-3.50 (m, 2H), 3.23 (m, 4H), 2.76-2.72 (m, 2H), 2.22-2.16 (m, 6H), 1.96-1.75 (m, 6H), 1.41-1.39 (m, 8H), 0.91-0.88 (m, 6H).

### Compound 2-7: Preparation of 4-(4-((4′-chloro-5,5-dimethyl-3,4,5,6-tetrahydro-[1,1′-biphenyl]-2-yl)methyl)piperazin-1-yl)-2-(2,3-dihydropyrrolo[3′,2′:5,6]pyrido[2,3-b][1,4]oxazin-1(6H)-yl)-N-((3-nitro-4-(((tetrahydro-2H-pyran-3-yl)methyl)amino)phenyl)sulfonyl)benzamide

Synthesis of 3-nitro-4-(((tetrahydro-2H-pyran-3-yl)methyl)amino)benzenesulfonamide: Into a 50-mL round-bottom flask, was placed (tetrahydro-2H-pyran-3-yl)methanamine (200 mg, 1.74 mmol, 1.00 equiv), THE (5 mL), 4-fluoro-3-nitrobenzene-1-sulfonamide (383 mg, 1.74 mmol, 1.00 equiv), Cs₂CO₃ (1.134 g, 3.48 mmol, 2.00 equiv). The resulting solution was stirred for 3h at 50 degrees C. in an oil bath. The resulting mixture was concentrated under vacuum. The residue was applied onto a silica gel column with ethyl acetate/petroleum ether (1/1). This resulted in 270 mg (49.3%) of 3-nitro-4-(((tetrahydro-2H-pyran-3-yl)methyl)amino) benzenesulfonamide as a yellow solid. LC-MS-PH-PHNW-4-41-1: (ES, m/z): M+1=316, R,T=1.25 min. The measurements of the retention were done with a reversed phase column (C18). Shimadzu LCMS 2020; 50*3.0 Kinetex 2.6u XB-ODS, 2.6 microm; Eluent A: water (0.5% FA); Eluent B: Acetonitrile(0.05% TFA); linear gradient from 5% acetonitrile to 100% acetonitrile in 2.6 minutes; Oven temperature 40° C.; flow:1.0 mL/min.

Synthesis of 4-(4-((4′-chloro-5,5-dimethyl-3,4,5,6-tetrahydro-[1,1′-biphenyl]-2-yl)methyl)piperazin-1-yl)-2-(2,3-dihydropyrrolo[3′,2′:5,6]pyrido[2,3-b][1,4]oxazin-1(6H)-yl)-N-((3-nitro-4-(((tetrahydro-2H-pyran-3-yl)methyl)amino)phenyl)sulfonyl)benzamide: Into a 100-mL round-bottom flask, was placed 4-(4-[[2-(4-chlorophenyl)-4,4-dimethylcyclohex-1-en-1-yl]methyl]piperazin-1-yl)-2-[13-oxa-2,4,10-triazatricyclo[7.4.0.0\hat{ }[3,7]]trideca-1,3(7),5,8-tetraen-10-yl]benzoic acid (50 mg, 0.082 mmol, 1.00 equiv) in dichloromethane (5 mL), EDCI (63 mg, 0.328 mmol, 4.00 equiv), 4-dimethylaminopyridine (20 mg, 0.164 mmol, 2.00 equiv), 3-nitro-4-(((tetrahydro-2H-pyran-4-yl)methyl)amino)benzenesulfonamide (26 mg, 0.082 mmol, 1.00 equiv). The resulting solution was stirred for overnight at 40° C. The resulting mixture was concentrated under vacuum. The crude product was purified by Prep-HPLC with the following conditions (Waters-2767): Column, X-bridge RP18, 5 um, 19*100 mm; mobile phase, 0.03% ammonia in water (0.03% NH₄HCO₃ & NH₄OH) and CH₃CN (32% CH₃CN up to 52% in 6 min); Detector, UV 254 nm. This resulted in 12.8 mg (17%) of 4-(4-((4′-chloro-5,5-dimethyl-3,4,5,6-tetrahydro-[1,1′-biphenyl]-2-yl)methyl)piperazin-1-yl)-2-(2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide as a yellow solid. LC-MS-PH-PHNW-4-41-0: (ES, m/z): M+1=909, R,T=1.60 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.0 minutes; Oven temperature 40° C.; flow:1.5 mL/min. 1H NMR (300 MHz, Chloroform-d) δ 12.40 (s, 1H), 8.72 (s, 1H), 8.63 (s, 1H), 8.44 (t, J=5.5 Hz, 1H), 8.10 (d, J=9.0 Hz, 1H), 7.93-7.82 (m, 1H), 7.25 (s, 4H), 7.13 (t, J=2.9 Hz, 1H), 7.00-6.68 (m, 6H), 6.52 (s, 1H), 6.17 (d, J=3.3 Hz, 1H), 4.71 (dd, J=23.6, 10.7 Hz, 2H), 3.99-3.79 (m, 3H), 3.70 (s, 1H), 3.57 (dd, J=18.5, 10.9 Hz, 3H), 3.46-3.17 (m, 7H), 2.80 (s, 2H), 2.29 (s, 3H), 2.21 (s, 2H), 2.03 (d, J=12.3 Hz, 5H), 1.75-1.60 (m, 4H), 1.44 (d, J=8.7 Hz, 3H), 1.28 (s, 1H), 0.97 (s, 6H), 0.87 (s, 1H). The measurements of the NMR spectra were done with BrukerAvanceIII HD 300 MHz with a probe head of BBOF.

### Compound 2-8: Preparation of (S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide

Synthesis of 6-(tert-butoxy)-N-(diphenylmethylidene)-1-[[2-(trimethylsilyl)ethoxy]methyl]-1H-pyrrolo[2,3-b]pyridin-5-amine: Into a 250-mL round-bottom flask, was placed a solution of 5-bromo-6-fluoro-1-[[2-(trimethylsilyl)ethoxy]methyl]-1H-pyrrolo[2,3-b]pyridine (20.7 g, 60 mmol, 1.00 equiv) in dioxane (300 mL), t-BuOK (20.5 g, 180 mmol, 3.00 equiv), xantphos (6.9 g, 12 mmol, 0.20 equiv), Pd₂(dba)₃.CHCl₃ (5.7 g, 0.10 equiv), diphenylmethanimine (14.04 g, 78 mmol, 1.20 equiv). The resulting solution was stirred for overnight at 100° C. The resulting mixture was concentrated under vacuum. The residue was applied onto a silica gel column with ethyl acetate/petroleum ether (0:1-1:20). This resulted in 15 g (crude) of 6-(tert-butoxy)-N-(diphenylmethylidene)-1-[[2-(trimethylsilyl)ethoxy]methyl]-1H-pyrrolo[2,3-b]pyridin-5-amine as white oil. LC-MS-PH-PHNW-4-7-9: (ES, m/z): M+1=500.

Synthesis of 5-amino-1-[[2-(trimethylsilyl)ethoxy]methyl]-1H-pyrrolo[2,3-b]pyridin-6-ol hydrogen chloride salt: Into a 500-mL round-bottom flask, was placed 6-(tert-butoxy)-N-(diphenylmethylidene)-1-[[2-(trimethylsilyl)ethoxy]methyl]-1H-pyrrolo[2,3-b]pyridin-5-amine (15 g, 30.02 mmol, 1.00 equiv) in dioxane (120 mL), HCl/dioxane(4M, 30 mL). The resulting solution was stirred for 5 h at room temperature. The resulting solution was diluted with 500 mL of ether. The solids were collected by filtration. This resulted in 5 g (crude) of 5-amino-1-[[2-(trimethylsilyl)ethoxy]methyl]-1H-pyrrolo[2,3-b]pyridin-6-ol hydrogen chloride salt as a red solid, Q-NMR show it might converted into 3 hydrogen chloride salt. LC-MS-PH-PHNW-4-10-1: (ES, m/z): M+1=280, R,T=0.811 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

### Synthesis of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-11-one: Into a 100-mL round-bottom flask, was placed 5-amino-1-[[2-(trimethylsilyl)ethoxy]methyl]-1H-pyrrolo[2,3-b]pyridin-6-ol (1.5 g, 5.37 mmol, 1 equiv), DMF (50 mL), K₂CO₃ (2.2 g, 16.11 mmol, 3 equiv). This was followed by the addition of 2-chloropropanoyl chloride (1.4 g, 10.74 mmol, 2 equiv) dropwise with stirring at 0° C. The resulting solution was stirred for overnight at room temperature. The reaction was then quenched by the addition of 50 mL of water. The resulting solution was extracted with 2×50 mL of ethyl acetate. The resulting mixture was washed with 2 x50 mL of brine. The mixture was dried over anhydrous sodium sulfate and filtrate and concentrated under vacuum. The residue was applied onto a silica gel column with ethyl acetate/petroleum ether (0:1-1:3). This resulted in 500 mg (27.93%) of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-11-one as a yellow solid. LC-MS-PH-PHNW-4-37-1: (ES, m/z): M+1=334, R,T=1.123 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-37-1: (CDCl₃,300 ppm): 8.34 (s, 1H), 7.63 (d, J=3 Hz, 1H), 7.42-7.28 (m, 1H), 6.46 (d, J=3 Hz, 1H), 5.68 (s, 2H), 4.92-4.85 (m, 1H), 3.63-3.53 (m, 2H), 1.85-1.81 (d, J=12 Hz, 3H), 0.94-0.89 (m, 2H), -0.154 (s, 9H)

Synthesis of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene & (12 S)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene & (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene: Into a 100-mL 3-necked round-bottom flask, was placed 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-11-one (500 mg, 1.50 mmol, 1 equiv), THE (20 mL). This was followed by the addition of LiAlH₄ (113.8 mg, 3.00 mmol, 2 equiv), in portions at 0° C. The resulting solution was stirred for 1 overnight at room temperature. The reaction was then quenched by the addition of 20 mL of water. The solids were filtered out. The resulting solution was extracted with 2×20 mL of ethyl acetate. The resulting mixture was washed with 2 x20 mL of brine. The mixture was dried over anhydrous sodium sulfate and filtrate and concentrated under vacuum. The residue was applied onto a silica gel column with ethyl acetate/petroleum ether (0:1-1:3). This resulted in 450 mg (93%) of 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene as a yellow solid. The crude 12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (450 mg) was purified by Chiral-Prep-HPLC with the following conditions: (SHIMADZU LC-20AT): Column, CHIRALPAK IC; mobile phase A:n-hexane, Phase B:ethanol; Detector, 220 nm. This resulted in 200 mg (44%) of (12S)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene as a yellow solid.

This resulted in 200 mg (44%) of (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene as a yellow solid. LC-MS-PH-PHNW-4-37-2: (ES, m/z): M+1=320, R,T=1.107 min.

The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR-PH-PHNW-4-37-2: (CDCl₃,300 ppm): 7.63 (s, 1H), 7.17 (s, 1H), 6.36-6.35 (d, J=3 Hz, 1H), 5.57-5.52 (m, 2H), 4.59-4.55 (m, 1H), 3.62-3.46 (m, 3H), 3.18-3.14 (m, 1H), 1.62-1.44 (m, 3H), 0.93-0.88 (m, 2H),-0.17 (s, 9H).

Synthesis of methyl 4-(4-[[2-(4-chlorophenyl)-4,4-dimethylcyclohex-1-en-1-yl]methyl]piperazin-1-yl)-2-[(12 S)-12-methyl-4-[[2-(trimethylsilyl)ethoxy]methyl]-13-oxa-2,4,10-triazatricyclo[7.4.0.0\hat{ }[3,7]]trideca-1(9),2,4,10-triazatricyclo[7.4.0.0\hat{ },7]trideca-1(9),2,5,7-tetraen-10-yl]benzoate: Into a 8-mL vial, was placed (12S)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (200 mg, 0.63 mmol, 1 equiv), methyl 2-bromo-4-(4-[[2-(4-chlorophenyl)-4,4-dimethylcyclohex-1-en-1-yl]methyl]piperazin-1-yl)benzoate (399.6 mg, 0.75 mmol, 1.2 equiv), Cs₂CO₃ (611.9 mg, 1.88 mmol, 3 equiv), Dioxane (5 mL), Chloro[9,9-dimethyl-4,5-bis(diphenylphosphino)xanthene][2-amino-1,1-biphenyl-2-yl]palladium(II) (120 mg). The resulting solution was stirred for 2 hr at 110° C. in an oil bath. The resulting mixture was concentrated under vacuum. The residue was applied onto a silica gel column with ethyl acetate/petroleum ether (0:1-1:10). This resulted in 250 mg (51.83%) of methyl 4-(4-[[2-(4-chlorophenyl)-4,4-dimethylcyclohex-1-en-1-yl]methyl]piperazin-1-yl)-2-[(12S)-12-methyl-4-[[2-(trimethylsilyl)ethoxy]methyl]-13-oxa-2,4,10-triazatricyclo[7.4.0.0\hat{ }[3,7]]trideca-1(9),2,4,10- triazatricyclo[7.4.0.0\hat{ },7]trideca-1(9),2,5,7-tetraen-10-yl]benzoate as a yellow solid.

Synthesis of methyl 4-(4-[[2-(4-chlorophenyl)-4,4-dimethylcyclohex-1-en-1-yl]methyl]piperazin-1-yl)-2-[(12S)-12-methyl-13-oxa-2,4,10-triazatricyclo[7.4.0.0\hat{ }[3,7]]trideca-1(9),2,5,7-tetraen-10-yl]benzoate. Into a 100-mL round-bottom flask, was placed methyl 4-(4-[[2-(4-chlorophenyl)-4,4-dimethylcyclohex-1-en-1-yl]methyl]piperazin-1-yl)-2-[(12S)-12-methyl-4-[[2-(trimethylsilyl)ethoxy]methyl]-13-oxa-2,4,10-triazatricyclo[7.4.0.0\hat{ }[3,7]]trideca-1(9),2 (250 mg, 0.32 mmol, 1 equiv), TBAF (3 g, 11.47 mmol, 35.36 equiv), THE (30 mL), ethane-1,2-diamine (2 g, 33.28 mmol, 102.56 equiv). The resulting solution was stirred for 12 h at 70° C. in an oil bath. The reaction was then quenched by the addition of 50 mL of water. The resulting solution was extracted with 2×50 mL of ethyl acetate. The resulting mixture was washed with 3 x50 mL of brine. The mixture was dried over anhydrous sodium sulfate and filtrate and concentrated under vacuum. The residue was applied onto a silica gel column with ethyl acetate/petroleum ether (0:1-1:3). This resulted in 90 mg (43%) of methyl 4-(4-[[2-(4-chlorophenyl)-4,4-dimethylcyclohex-1-en-1-yl]methyl]piperazin-1-yl)-2-[(12S)-12-methyl-13-oxa-2,4,10-triazatricyclo[7.4.0.0\hat{ }[3,7]]trideca-1(9),2,5,7-tetraen-10-yl]benzoate as a yellow solid. H-NMR-PH-PHNW-4-37-50: (CDCl₃,300 ppm): 8.24 (s, 1H), 7.89-7.86 (d, J=9 Hz, 1H), 7.32-7.24 (m, 6H), 7.14-7.01 (m, 1H), 6.97-6.94 (m, 2H), 6.73-6.65 (m, 2H), 6.18 (s, 1H), 3.67-3.53 (m, 3H), 3.32-3.18 (m, 3H), 2.98-2.80 (m, 1H), 2.30-2.04 (m, 6H), 1.99 (s, 2H), 1.56-1.50 (m, 5H), 0.91-0.88 (m, 6H).

Synthesis of 4-(4-[[2-(4-chlorophenyl)-4,4-dimethylcyclohex-1-en-1-yl]methyl]piperazin-1-yl)-2-[(12 S)-12-methyl-13-oxa-2,4,10-triazatricyclo[7.4.0.0\hat{ }[3,7]]trideca-1(9),2,5,7-tetraen-10-yl]benzoic acid. Into a 8-mL vial, was placed methyl 4-(4-[[2-(4-chlorophenyl)-4,4-dimethylcyclohex-1-en-1-yl]methyl]piperazin-1-yl)-2-[(12S)-12-methyl-13-oxa-2,4,10-triazatricyclo[7.4.0.0\hat{ }[3,7]]trideca-1(9),2,5,7-tetraen-10-yl]benzoate (90 mg, 0.14 mmol, 1 equiv), MeOH (1 mL), H₂O (1 mL), THF (1 mL), NaOH (22.5 mg, 0.56 mmol, 4 equiv). The resulting solution was stirred for overnight at 60° C. in an oil bath. The pH value of the solution was adjusted to 5 with HCl (1 mol/L). The resulting mixture was concentrated under vacuum. The residue was applied onto a silica gel column with dichloromethane/methanol (1:0-10:1). This resulted in 80 mg (90%) of 4-(4-[[2-(4-chlorophenyl)-4,4-dimethylcyclohex-1-en-1-yl]methyl]piperazin-1-yl)-2-[(12S)-12-methyl-13-oxa-2,4,10-triazatricyclo[7.4.0.0\hat{ }[3,7]]trideca-1(9),2,5,7-tetraen-10-yl]benzoic acid as a yellow solid. LC-MS: (ES, m/z): M+1=626, R,T=1.035 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min.

Synthesis of (S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide. Into a 8-mL vial, was placed 4-(4-[[2-(4-chlorophenyl)-4,4-dimethylcyclohex-1-en-1-yl]methyl]piperazin-1-yl)-2-[(12R or S)-12-methyl-13-oxa-2,4,10-triazatricyclo[7.4.0.0\hat{ }[3,7]]trideca-1(9),2,5,7-tetraen-10-yl]benzoic acid (90 mg, 0.14 mmol, 1 equiv), 4-[[(4-fluorooxan-4-yl)methyl]amino]-3-nitrobenzene-1-sulfonamide (57.5 mg, 0.17 mmol, 1.2 equiv), DCM (5 mL), DMAP (70.2 mg, 0.57 mmol, 4 equiv), EDCI (55.1 mg, 0.29 mmol, 2.00 equiv). The resulting solution was stirred for overnight at room temperature. The resulting mixture was concentrated. The crude product was purified by Flash-Prep-HPLC with the following conditions (IntelFlash-1): Column, C18 silica gel; mobile phase, Water(0.I % FA) and ACN (48.0% ACN up to 53.0% in 7 min, hold 95.0% in 1 min, down to 48.0% in 1 min, hold 48.0% in 1 min) within 5 min; Detector, UV 254 nm. This resulted in 22 mg (20%) of (S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide. LC-MS: (ES, m/z): M+1=923, R,T=2.653 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.5 minutes; Oven temperature 40° C.; flow:1.5 mL/min. H-NMR: (d-CDCl₃, 300 ppm): 8.62 (s, 1H), 8.44-8.42 (m, 2H), 8.09-8.06 (m, 1H), 7.85-7.70 (m, 1H), 7.28-7.22 (m, 2H), 6.94-6.80 (m, 3H), 6.72-6.62 (m, 2H), 6.10 (s, 1H), 4.05-4.00 (m, 2H), 3.50-3.17 (m, 10H), 2.95-2.35 (m, 2H), 2.27-2.19 (m, 4H), 1.98-1.70 (m, 5H), 1.60-1.26 (m, 11H), 0.95 (s, 6H).

### Compound 2-9: Preparation of (R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide

Synthesis of N-[(2R)-2-hydroxypropyl]-4-methylbenzene-1-sulfonamide: Into a 250-mL round-bottom flask, was placed (2R)-1-aminopropan-2-ol (5 g, 1 equiv), DCM (70 mL), TsCl (12.67 g, 1 equiv). This was followed by the addition of TEA (7.41 g, 1.1 equiv) at 0° C. The resulting solution was stirred for 3 hr at 0° C. The resulting mixture was concentrated. The residue was applied onto a silica gel column with ethyl acetate/petroleum ether (1/1). This resulted in 8 g of N-(2R)-2-hydroxypropyl]-4-methylbenzene-1-sulfonamide as a white solid. ¹H NMR ((300 MHz, DMSO-d₆, ppm): δ7.74-7.63 (m, 2H), 7.52-7.34 (m, 3H), 4.66 (d, J=4.7 Hz, 1H), 3.58 (qd, J=6.3, 4.8 Hz, 1H), 2.73-2.50 (m, 2H), 2.39 (s, 3H), 0.99 (d, J=6.2 Hz, 3H).

Synthesis of methyl 2-bromo-4-(4-[[2-(4-chlorophenyl)-4,4-dimethylcyclohex-1-en-1-yl]methyl]piperazin-1-yl)benzoate: Into a 250-mL round-bottom flask, was placed a solution of 1-[[2-(4-chlorophenyl)-4,4-dimethylcyclohex-1-en-1-yl]methyl]piperazine (15.09 g, 47.32 mmol, 1.00 equiv) in DMA (150 mL), DIEA (12.9 g, 99.81 mmol, 2.00 equiv), methyl 2-bromo-4-fluorobenzoate (11.6 g, 49.78 mmol, 1.00 equiv). The resulting solution was stirred for 12 h at 100 degree. The reaction mixture was cooled to room temperature. The reaction was then quenched by the addition of 50 mL of water. The resulting solution was extracted with 3×100 mL of ethyl acetate and the organic layers combined. The resulting mixture was washed with 3×100 mL of brine. The mixture was dried over anhydrous sodium sulfate, then filtered and concentrated under vacuum. The residue was applied onto a silica gel column with ethyl acetate/petroleum ether (0:1-1:5). This resulted in 7 g (crude) of methyl 2-bromo-4-(4-[[2-(4-chlorophenyl)-4,4-dimethylcyclohex-1-en-1-yl]methyl]piperazin-1-yl)benzoate as yellow oil. LC-MS: (ES, m/z): M+1=533, 531.

Synthesis of N-[(2R)-2-[(5-bromo-1-[[2-(trimethylsilyl)ethoxy]methyl]-1H-pyrrolo[2,3-b]pyridin-6-yl)oxy]propyl]-4-methylbenzene-1-sulfonamide: Into a 100-mL round-bottom flask, was placed N-[(2R)-2-hydroxypropyl]-4-methylbenzene-1-sulfonamide (1 g, 1.5 equiv), DMF (3 mL). This was followed by the addition of NaH (0.35 g, 3 equiv) at 0° C. in 10 min. To this was added 5-bromo-6-fluoro-1-[[2-(trimethylsilyl)ethoxy]methyl]-1H-pyrrolo[2,3-b]pyridine (1 g, 1 equiv) at 0° C. The resulting solution was stirred for 4 hr at room temperature. The reaction was then quenched by the addition of 20 mL of water. The resulting solution was extracted with 3×50 mL of ethyl acetate and the organic layers combined and concentrated. The residue was applied onto a silica gel column with ethyl acetate/petroleum ether (⅓). This resulted in 400 mg of N-[(2R)-2-[(5-bromo-1-[[2-(trimethylsilyl)ethoxy]methyl]-1H-pyrrolo[2,3-b]pyridin-6-yl)oxy]propyl]-4-methylbenzene-1-sulfonamide as a yellow solid. ¹H-NMR (300 MHz, DMSO-d₆, ppm): δ8.18 (s, 1H), 7.72-7.62 (m, 2H), 7.41 (d, J=3.5 Hz, 1H), 7.28 (d, J=8.0 Hz, 2H), 6.42 (d, J=3.6 Hz, 1H), 5.50 (q, J=10.8 Hz, 2H), 5.16 (q, J=6.1 Hz, 1H), 3.53-3.38 (m, 2H), 3.13 (dd, J=13.4, 6.0 Hz, 1H), 2.98 (dd, J=13.4, 5.8 Hz, 1H), 2.29 (s, 3H), 1.28 (d, J=6.2 Hz, 3H), 0.80 (ddt, J=16.1, 14.1, 7.1 Hz, 2H), 0.13 (s, 9H).

### Synthesis of (12R)-12-methyl-10-(4-methylbenzenesulfonyl)-4-[[2-(trimethylsilyl)ethoxy]methyl]-13-oxa-2,4,10-triazatricyclo[7.4.0.0\hat{ }[3,7]]trideca-1(9),2,5,7-tetraene: Into a 100-mL round-bottom flask purged and maintained with an inert atmosphere of nitrogen, was placed N-[(2R)-2-[(5-bromo-1-[[2-(trimethylsilyl)ethoxy]methyl]-1H-pyrrolo[2,3-b]pyridin-6-yl)oxy]propyl]-4-methylbenzene-1-sulfonamide (3.9 g, 1 equiv), dioxane (50 mL), t-BuXPhos 3G (560 mg, 0.1 equiv), Cs₂O₃ (6.9 g, 3 equiv). The resulting solution was stirred for 4 hr at 90° C. The resulting mixture was concentrated. The residue was applied onto a silica gel column with ethyl acetate/petroleum ether (⅓). This resulted in 3.3 g of (12R)-12-methyl-10-(4-methylbenzenesulfonyl)-4-[[2-(trimethylsilyl)ethoxy]methyl]-13-oxa-2,4,10-triazatricyclo[7.4.0.0\hat{ }[3,7]]trideca-1(9),2,5,7-tetraene as colorless oil. ¹H-NMR (300 MHz, DMSO-d₆, ppm): δ8.28 (s, 1H), 7.56-7.39 (m, 3H), 7.39-7.30 (m, 2H), 6.52 (d, J=3.6 Hz, 1H), 5.43 (s, 2H), 4.28 (dd, J=14.6, 2.6 Hz, 1H), 3.61-3.41 (m, 3H), 3.26-3.11 (m, 1H), 2.35 (s, 3H), 1.32-1.11 (m, 3H), 0.89-0.74 (m, 2H), 0.11 (s, 9H)

Synthesis of (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene: Into a 100-mL 3-necked round-bottom flask, was placed Na (1.3 1 g, 9.4 equiv), naphthalene (0.76 g, 6 equiv). This was followed by the addition of DME (15 mL) for 30 min at room temperature. To this was added the solution (12R)-12-methyl-10-(4-methylbenzenesulfonyl)-4-[[2-(trimethylsilyl)ethoxy]methyl]-13-oxa-2,4,10-triazatricyclo[7.4.0.0\hat{ }[3,7]]trideca-1(9),2,5,7-tetraene (2.8 g, 1 equiv) in THF (15 mL) at ˜78° C. The resulting solution was stirred for 3 hr at room temperature. The reaction was then quenched by the addition of 30 mL of NH₄Cl. The resulting solution was extracted with 3×100 mL of ethyl acetate and the organic layers combined and concentrated. The residue was applied onto a silica gel column with ethyl acetate/petroleum ether (⅓). This resulted in 1.4 g of (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene as a colorless oil. LC-MS (ES, m/z): 320 [M+1]*; RT=1.61 min.

Synthesis of methyl 4-(4-[[2-(4-chlorophenyl)-4,4-dimethylcyclohex-1-en-1-yl]methyl]piperazin-1-yl)-2-[(12S)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzoate: Into a 250-mL round-bottom flask purged and maintained with an inert atmosphere of nitrogen, was placed (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (1.178 g, 1 equiv), methyl 2-bromo-4-(4-[[2-(4-chlorophenyl)-4,4-dimethylcyclohex-1-en-1-yl]methyl]piperazin-1-yl)benzoate (2.35 g, 1.2 equiv), dioxane (15 mg, 15 equiv), Cs₂CO₃ (3.62 g, 3 equiv), XantPhos (328 mg, 0.1 equiv). The resulting solution was stirred for 2 hr at 110° C. The resulting mixture was concentrated. The residue was applied onto a silica gel column with ethyl acetate/petroleum ether (⅓). This resulted in 3.2 g of methyl 4-(4-[[2-(4-chlorophenyl)-4,4-dimethylcyclohex-1-en-1-yl]methyl]piperazin-1-yl)-2-[(12S)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzoate as a yellow solid. LC-MS (ES, m/z): 770 [M+1]*; RT=1.33 min.

Synthesis of methyl 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoate: Into a 250-mL round-bottom flask, was placed methyl 4-(4-[[2-(4-chlorophenyl)-4,4-dimethylcyclohex-1-en-1-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzoate (3.2 g, 1 equiv), THE (50 mL), TBAF (20 g), ethane-1,2-diamine (33 g). The resulting solution was stirred for 5 hr at 70° C. The reaction was then quenched by the addition of 50 mL of water. The resulting solution was extracted with 3×100 mL of ethyl acetate and the organic layers combined and concentrated. The residue was applied onto a silica gel column with dichloromethane/methanol (10/1). This resulted in 2.2 g of methyl 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoate as a yellow solid. LC-MS-(ES, m/z): 640 [M+1]⁺; RT=2.51 min.

Synthesis of 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid: Into a 250-mL round-bottom flask, was placed methyl 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoate (2.2 g, 1 equiv), MEOH/H₂O/THF (10 mL/10 ml/10 mL). The resulting solution was stirred for 5 hr at 60° C. The resulting mixture was concentrated. The resulting solution was extracted with 3×100 mL of dichloromethane and the organic layers combined and concentrated. The residue was applied onto a silica gel column with dichloromethane/methanol (10/1). This resulted in 1.6 g of 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid as yellow oil. LC-MS (ES, m/z): 626 [M+1]⁺; RT=2.47 min.

Synthesis of 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-(3-nitro-4-[[(oxan-4-yl)methyl]amino]benzenesulfonyl)benzamide: Into a 250-mL round-bottom flask, was placed 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid (1.6 g, 1 equiv), DCM (20 mL), 3-nitro-4-[[(oxan-4-yl)methyl]amino]benzene-1-sulfonamide (890 mg, 1.2 equiv), DMAP (1.25 g, 4 equiv), EDCI (980 mg, 2 equiv). The resulting solution was stirred for 14 hr at room temperature. The resulting mixture was concentrated. The residue was applied onto a silica gel column with EA/DCM ( 1/10). This resulted in 0.82 g of 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-(3-nitro-4-[[(oxan-4-yl)methyl]amino]benzenesulfonyl)benzamide as a yellow solid. LC-MS (ES, m/z): 923 [M+1]*; RT=3.50 min. ¹H NMR (300 MHz, CDCl₃, ppm): δ12.48 (s, 1H), 8.62 (d, J=2.4 Hz, 2H), 8.54-8.38 (m, 1H), 8.16-7.95 (m, 1H), 7.81-7.71 (m, 1H), 7.32-7.12 (m, 6H), 7.07 (t, J=2.9 Hz, 1H), 6.97-6.77 (m, 3H), 6.76-6.60 (m, 2H), 6.52 (s, 1H), 6.09 (d, J=3.0 Hz, 1H), 4.88 (d, J=7.7 Hz, 1H), 4.02 (dd, J=11.8, 4.2 Hz, 2H), 3.55-3.34 (m, 4H), 3.32-3.14 (m, 5H), 3.08 (s, 1H), 2.77 (d, J=9.6 Hz, 2H), 2.28 (s, 3H), 2.19 (s, 2H), 1.99 (d, J=7.6 Hz, 4H), 1.72 (d, J=12.7 Hz, 2H), 1.45 (ddd, J=24.5, 12.3, 5.8 Hz, 6H), 0.94 (d, J=2.1 Hz, 6H).

### Compound 2-10: Preparation of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[13-thia-2,4,10-triazatricyclo[7.4.0.0\hat{ }[3,7]]trideca-1(9),2,5,7-tetraen-10-yl]benzamide

Into a 250-mL round-bottom flask, was placed cysteamine hydrochloride (5.00 g, 44.014 mmol, 1.00equiv), DCM (100.00 mL), TEA (13.36 g, 0.132 mmol, 3equiv), acetic anhydride (4.94 g, 0.048 mmol, 1.1 equiv). The resulting solution was stirred for overnight at room temperature. The reaction was then quenched by the addition of 100 mL of water. The resulting solution was extracted with 3×50 mL of ethyl acetate dried over anhydrous sodium sulfate. The resulting solution was concentrated under vacuum. The residue was applied onto a silica gel column and eluted with ethyl acetate/petroleum ether (7:1). This resulted in 7.2 g (crude) of 2-acetamidoethanethiol as light yellow oil. ¹H ¹H NMR (300 MHz, DMSO-d₆) δ 7.99 (s, 1H), 3.20-3.214 (m, 2H), 2.54-2.47 (m, 2H), 1.90 (s, 1H), 1.81 (s, 3H).

Into a 100-mL round-bottom flask, was placed 2-acetamidoethanethiol (1.19 g, 9.985 mmol, 1.00equiv), DMF (30.00 mL), 5-bromo-6-fluoro-1-[[2-(trimethylsilyl)ethoxy]methyl]pyrrolo[2,3-b]pyridine (3.45 g, 0.010 mmol, 1 equiv), Cs₂CO₃ (9.76 g, 0.030 mmol, 3equiv). The resulting solution was stirred for 5 h at 80 degrees C. in an oil bath. The reaction mixture was cooled to room temperature with a water/ice bath. The reaction was then quenched by the addition of 50 mL of water. The resulting solution was extracted with 3×50 mL of ethyl acetate dried over anhydrous sodium sulfate and concentrated. The residue was applied onto a silica gel column and eluted with ethyl acetate/petroleum ether (1:1). The collected fractions were combined and concentrated. This resulted in 2 g (45.07%) of N-[2-[(5-bromo-1-[[2-(trimethylsilyl)ethoxy]methyl]pyrrolo[2,3-b]pyridin-6-yl)sulfanyl]ethyl]acetamide as yellow oil. LCMS-PH-PHNW-4-121-2 (ES, m/z): M+1: 444/446.

Into a 50-mL round-bottom flask purged and maintained with an inert atmosphere of nitrogen, was placed N-[2-[(5-bromo-1-[[2-(trimethylsilyl)ethoxy]methyl]pyrrolo[2,3-b]pyridin-6-yl)sulfanyl]ethyl]acetamide (2.00 g, 0.004 mmol, 1.00equiv), DMF(30 ml), Cs₂CO₃ (4.40 g, 0.013 mmol, 3equiv), BrettPhos Pd G3 Precatalyst (0.61 mg, 0.001 mmol, 0.15equiv). The resulting solution was stirred for overnight at 100 degrees C. in an oil bath. The reaction was cooled down to r.t then quenched by the addition of 30 mL of water. The resulting solution was extracted with 3×50 mL of ethyl acetate dried over anhydrous sodium sulfate and concentrated. The residue was applied onto a silica gel column and eluted with ethyl acetate/petroleum ether (5:2). The collected fractions were combined and concentrated. This resulted in 670 mg (40.96%) of 1-(4-[[2-(trimethylsilyl)ethoxy]methyl]-13-thia-2,4,10-triazatricyclo[7.4.0.0\hat{ }[3,7]]trideca-1(9),2,5,7-tetraen-10-yl)ethanone as yellow oil. LCMS-PH-PHNW-4-121-3 (ES, m/z): M+1: 364.

Into a 40-mL vial, was placed 1-(4-[[2-(trimethylsilyl)ethoxy]methyl]-13-thia-2,4,10-triazatricyclo[7.4.0.0\hat{ }[3,7]]trideca-1(9),2,5,7-tetraen-10-yl)ethanone (600.00 mg, 1.650 mmol, 1.00equiv), MeOH (10.00 mL), NaOH(4M) (10.00 mL). The resulting solution was stirred for overnight at 80 degrees C. in an oil bath. The resulting mixture was concentrated. The resulting solution was extracted with 3×30 L of ethyl acetate dried over anhydrous sodium sulfate and concentrated. The residue was applied onto a silica gel column and eluted with ethyl acetate/petroleum ether (3:1). The collected fractions were combined and concentrated. This resulted in 410 mg (77.27%) of 4-[[2-(trimethylsilyl)ethoxy]methyl]-13-thia-2,4,10-triazatricyclo[7.4.0.0\hat{ }[3,7]]trideca-1(9),2,5,7-tetraene as yellow oil. LC-MS-PH-PHNW-4-121-4: (ES, m/z): M+1:322.

Into a 100-mL round-bottom flask purged and maintained with an inert atmosphere of nitrogen, was placed 4-[[2-(trimethylsilyl)ethoxy]methyl]-13-thia-2,4,10-triazatricyclo[7.4.0.0\hat{ }[3,7]]trideca-1(9),2,5,7-tetraene (200.00 mg, 0.622 mmol, 1.00equiv), 2-bromo-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide (1016.69 mg, 1.244 mmol, 2equiv), DMF (20.00 mL), Cs₂CO₃ (608.04 mg, 1.866 mmol, 3equiv), CuI (47.39 mg, 0.249 mmol, 0.4equiv), 1,10-phenanthroline (22.42 mg, 0.124 mmol, 0.2equiv). The resulting solution was stirred for 4 h at 95 degrees C. in an oil bath. The reaction was then quenched by the addition of 30 mL of water. The solids were filtered out. The resulting solution was extracted with 3×20 mL of ethyl acetate concentrated. The residue was applied onto a silica gel column and eluted with ethyl acetate/petroleum ether (2:1). The collected fractions were combined and concentrated. This resulted in 300 mg (45.59%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-13-thia-2,4,10-triazatricyclo[7.4.0.0\hat{ }[3,7]]trideca-1(9),2,5,7-tetraen-10-yl)benzamide as yellow oil. LC-MS-PH-PHNW-4-121-5: (ES, m/z): M+1:1057.

Into a 100-mL round-bottom flask, was placed 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-13-thia-2,4,10-triazatricyclo[7.4.0.0\hat{ }[3,7]]trideca-1(9),2,5,7-tetraen-10-yl)benzamide (300.00 mg, 0.284 mmol, 1.00equiv), THE (20.00 mL, 0.277 mmol, 0.98equiv), TBAF (741.53 mg, 2.836 mmol, 10.00equiv), ethane-1,2-diamine (170.44 mg, 2.836 mmol, 10equiv). The resulting solution was stirred for overnight at 70 degrees C. in an oil bath. The resulting mixture was concentrated. The crude product was purified by Chiral-Prep-HPLC with the following conditions:Column, XBridge Prep C18 OBD 19*150 mm 5 um; mobile phase, A: 0.1% HCl in water; B: ACN; Gradient: 24-95% B in 7.9 min; Flow rate: 20 ml/min; Detector, 220 nm. This resulted in 50 mg (18.29%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[13-thia-2,4,10-triazatricyclo[7.4.0.0\hat{ }[3,7]]trideca-1(9),2,5,7-tetraen-10-yl]benzamide hydrochloride as a yellow solid. LC-MS-PH-PHNW-4-121-0: (ES, m/z): M+1-HCl: 963. H-NMR-PH-PHNW-4-121-0: ‘H’H NMR (300 MHz, DMSO-d₆) δ 8.35 (d, J=2.4 Hz, 1H), 7.53 (d, J=8.7 Hz, 1H), 7.39 (d, J=8.4 Hz, 2H), 7.32 (dd, J=9.3, 2.4 Hz, 1H), 7.15-7.06 (m, 3H), 6.86 (d, J=8.7 Hz, 1H), 6.74 (s, 1H), 6.62 (d, J=9.3 Hz, 1H), 6.45 (s, 1H), 5.94 (d, J=3.3 Hz, 1H), 3.88-3.74 (m, 6H), 3.61 (d, J=9.9 Hz, 4H), 3.49 (dd, J=12.0, 9.6 Hz, 2H), 3.43-3.26 (m, 7H), 3.15 (s, 2H), 2.74 (s, 2H), 2.21 (s, 2H), 2.03 (s, 2H), 1.45 (d, J=6.9 Hz, 2H), 0.94 (s, 6H).

### Compound 2-11: Preparation of 4-(4-[[2-(4-chlorophenyl)-4,4-dimethylcyclohex-1-en-1-yl]methyl]piperazin-1-yl)-2-(3,4-dihydro-2H-1,4-benzoxazin-4-yl)-N-(3-nitro-4-[[(oxan-4-yl)methyl]amino]benzenesulfonyl)benzamide

Into a 50-mL round-bottom flask purged and maintained with an inert atmosphere of nitrogen, was placed 2-bromo-4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)benzamide (1568.13 mg, 1.924 mmol, 1.30equiv), 3,4-dihydro-2H-1,4-benzoxazine (200 mg, 1.480 mmol, 1.00equiv), DMF (20.00 mL), CS₂CO₃ (1.45 g, 4.450 mmol, 3.01equiv), CuI (112.00 mg, 0.588 mmol, 0.40equiv), 1,10-phenanthroline (53.30 mg, 0.296 mmol, 0.20equiv). The resulting solution was stirred for 5 h at 90 degrees C. in an oil bath. The solids were filtered out. The resulting solution was extracted with 3×40 mL of ethyl acetate concentrated. The residue was applied onto a silica gel column and eluted with ethyl acetate/petroleum ether (5:1). The collected fractions were combined and concentrated. This resulted in 100 mg (7.77%) of 4-(4-[[2-(4-chlorophenyl)-4,4-dimethylcyclohex-1-en-1-yl]methyl]piperazin-1-yl)-2-(3,4-dihydro-2H-1,4-benzoxazin-4-yl)-N-(3-nitro-4-[[(oxan-4-yl)methyl]amino]benzenesulfonyl)benzamide as a yellow solid. LC-MS-PH-PHNW-4-101-0: (ES, m/z): M+1: 869. H-NMR-PH-PHNW-4-101-0: ¹H NMR (300 MHz, DMSO-d₆) δ 11.87 (s, 1H), 8.64 (t, J=6.0 Hz, 1H), 8.36 (d, J=2.4 Hz, 1H), 7.69-7.56 (m, 1H), 7.48 (d, J=8.7 Hz, 1H), 7.41-7.32 (m, 2H), 7.13-7.01 (m, 3H), 6.81 (dd, J=9.0, 2.4 Hz, 1H), 6.73 (d, J=2.4 Hz, 1H), 6.58 (dd, J=7.8, 1.8 Hz, 1H), 6.37 (dtd, J=20.1, 7.5, 1.8 Hz, 2H), 6.09 (dd, J=7.8, 1.8 Hz, 1H), 4.22 (s, 2H), 3.88 (dd, J=11.4, 4.2 Hz, 2H), 3.54 (s, 2H), 3.35 (d, J=7.2 Hz, 2H), 3.30 (s, 2H), 3.21 (s, 4H), 2.81 (s, 2H), 2.23 (d, J=24.3 Hz, 6H), 1.99 (d, J=2.1 Hz, 3H), 1.66 (d, J=12.6 Hz, 2H), 1.42 (t, J=6.3 Hz, 2H), 1.30 (tt, J=12.5, 6.3 Hz, 2H), 0.95 (s, 6H).

### Compound 2-12. Preparation of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[2H,3H-pyrido[4,3-b][1,4]oxazin-4-yl]benzamide

Into a 8-mL vial purged and maintained with an inert atmosphere of nitrogen, was placed 2-bromo-4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]benzamide (233.51 mg, 0.286 mmol, 1.30equiv), DMF (4.00 mL), 2H,3H,4H-pyrido[4,3-b][1,4]oxazine (30.00 mg, 0.220 mmol, 1.00equiv), 1,10-phenanthroline (7.94 mg, 0.044 mmol, 0.2equiv), CuI (16.79 mg, 0.088 mmol, 0.4equiv), Cs₂CO₃ (215.37 mg, 0.661 mmol, 3equiv). The resulting solution was stirred for 3 h at 95 degrees C. in an oil bath. The solids were filtered out. The resulting solution was quenched with 10 mL water and extracted with 3×10 mL of ethyl acetate concentrated. The crude product was purified by Prep-HPLC with the following conditions (2 #SHIMADZU (HPLC-01)): Column, SunFire Prep C18 OBD Column, 19*150 mm Sum 10 nm; mobile phase, Water(0.1% HCl) and ACN (20% PhaseB up to 40% in 8 min); Detector, UV=254 nm. This resulted in 8 mg (4.17%) of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[2H,3H-pyrido[4,3-b][1,4]oxazin-4-yl]benzamide as a yellow solid as HCl salt. LC-MS-PH-PHNW-4-104-0: (ES, m/z): M+1-HCl: 870. H-NMR-PH-PHNW-4-104-0: ¹H NMR (300 MHz, DMSO-d₆) δ 12.30 (s, 1H), δ10.37 (s, 1H), 8.64 (d, J=6.3 Hz, 1H), 8.43 (d, J=2.4 Hz, 1H), 7.88 (d, J=6.3 Hz, 1H), 7.80-7.65 (m, 1H), 7.54 (d, J=9.3 Hz, 1H), 7.42 (d, J=8.1 Hz, 2H), 7.30 (s, 1H), 7.19-7.09 (m, 4H), 6.96 (s, 2H), 4.58 (s, 1H), 4.31 (s, 1H), 3.89 (dd, J=11.7, 4.2 Hz, 4H), 3.66-3.56 (m, 4H), 3.29 (d, J=12.0 Hz, 6H), 2.76 (d, J=16.8 Hz, 2H), 2.42-2.24 (m, 4H), 2.06 (s, 2H), 1.95 (s, 1H), 1.66 (d, J=13.2 Hz, 2H), 1.49 (d, J=6.6 Hz, 2H), 1.40-1.21 (m, 2H), 0.97 (s, 6H).

### Compound 2-13: Preparation of 4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((S)-5-nitro-3-(tetrahydro-2H-pyran-4-yl)-3,4-dihydro-2H-benzo[b][1,4]oxazin-7-yl)sulfonyl)benzamide, and 4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((R)-5-nitro-3-(tetrahydro-2H-pyran-4-yl)-3,4-dihydro-2H-benzo[b][1,4]oxazin-7-yl)sulfonyl)benzamide

### Synthesis of 3-bromo-4-chloro-5-nitrobenzenesulfonamide, Into a 50-mL round-bottom flask, was placed 4-chloro-3-nitrobenzenesulfonamide (20.00 g, 84.520 mmol, 1.00 equiv), H₂SO₄ (20.00 mL). This was followed by the addition of NBS (22.56 g, 126.753 mmol, 1.50 equiv), in portions at 50 degrees C. The resulting solution was stirred for 2 hr at 60 degrees C. The resulting solution was diluted with 200 mL of cold H₂O. The resulting solution was extracted with 3×30 mL of ethyl acetate. The resulting mixture was washed with 1×20 ml of H₂O. The resulting mixture was washed with 1×20 mL of NaCl (aq.). The residue was applied onto a silica gel column with ethyl and eluted with ethyl acetate/petroleum ether (1:3). This resulted in 5 g (18.75%) of 3-bromo-4-chloro-5- nitrobenzenesulfonamide as a white solid. LC-MS-1: (ES, m/z): 312.9 [M−H]

Synthesis of 2-amino-2-(oxan-4-yl)ethanol: Into a 100-mL round-bottom flask, was placed amino(oxan-4-yl)acetic acid (4.00 g, 25.128 mmol, 1.00 equiv), THE (50.00 mL), LiAlH₄ (1.91 g, 50.324 mmol, 2.00 equiv). The resulting solution was stirred for 3 hr at 60 degrees C. in an oil bath. The reaction was then quenched by the addition of 10 mL of water. The resulting mixture was concentrated. This resulted in 6 g (crude) of 2-amino-2-(oxan-4-yl)ethanol as a white solid. LC-MS-21: (ES, m/z): 146.2 [M+H]⁺

Synthesis of 3-bromo-4-[[2-hydroxy-1-(oxan-4-yl)ethyl]amino]-5-nitrobenzenesulfonamide: Into a 100-mL round-bottom flask, was placed 3-bromo-4-chloro-5-nitrobenzenesulfonamide (5.00 g, 15.847 mmol, 1.00 equiv), 2-amino-2-(oxan-4-yl)ethanol (5.00 g), CH₃CN (30.00 mL, 0.731 mmol), DIEA (6.10 g, 47.198 mmol, 2.98 equiv). The resulting solution was stirred for 48 hr at 80 degrees C. in an oil bath. The resulting mixture was concentrated. The residue was applied onto a silica gel column and eluted with dichloromethane/methanol (10:1). This resulted in 1.2 g (17.85%) of 3-bromo-4-[[2-hydroxy-1-(oxan-4-yl) ethyl]amino]-5-nitrobenzenesulfonamide as a yellow solid. LC-MS-2: (ES, m/z): 424.1 [M+H]⁺.

Synthesis of methyl 4-(4-[[2-(4-chlorophenyl)-4,4-dimethylcyclohex-1-en-1-yl]methyl]piperazin-1-yl) -2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzoate. Into a 50-mL round-bottom flask, was placed (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (500.00 mg, 1.565 mmol, 1.00 equiv), methyl 2-bromo-4-(4-[[2-(4-chlorophenyl)-4,4-dimethylcyclohex-1-en-1-yl]methyl]piperazin-1-yl)benzoate (832.48 mg, 1.565 mmol, 1.00 equiv), xantphos Pd 2G (138.82 mg, 0.157 mmol, 0.10 equiv), Cs₂CO₃ (1529.77 mg, 4.695 mmol, 3.00 equiv), dioxane (10.00 mL). The resulting solution was stirred for 14 hr at 80 degrees C. The resulting mixture was concentrated. The residue was applied onto a silica gel column and eluted with ethyl acetate/petroleum ether (1:3). This resulted in 611 mg (50.67%) of methyl 4-(4-[[2-(4-chlorophenyl)-4,4-dimethylcyclohex-1-en-1-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzoate as a yellow solid. LC-MS-11: (ES, m/z):770.4 [M+H]⁺.

### Synthesis of methyl 4-(4-[[2-(4-chlorophenyl)-4,4-dimethylcyclohex-1-en-1-yl]methyl]piperazin-1-yl) -2-[(12R)-12-methyl-13-oxa-2,4,10-triazatricyclo[7.4.0.0\hat{ }[3,7]]trideca-1(9),2,5,7-tetraen-10-yl]benzoate. Into a 8-mL vial, was placed methyl 4-(4-[[2-(4-chlorophenyl)-4,4-dimethylcyclohex-1-en-1-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzoate (300.00 mg, 0.389 mmol,), ethane-1,2-diamine (0.20 mL), TBAF in TIF(1 ml, 2M). The resulting solution was stirred for 24 hr at 70 degrees C. in an oil bath. The resulting mixture was concentrated. The residue was applied onto a silica gel column with ethyl acetate/petroleum ether (1:1). This resulted in 100 mg (40.12%) of methyl 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoate as a white solid. LC-MS-11: (ES, m/z):640.3 [M+H]⁺

Synthesis of 4-(4-[[2-(4-chlorophenyl)-4,4-dimethylcyclohex-1-en-1-yl]methyl]piperazin-1-yl)-2-[(12R) -12-methyl-13-oxa-2,4,10-triazatricyclo[7.4.0.0\hat{ }[3,7]]trideca-1(9),2,5,7-tetraen-10-yl]benzoic acid. Into a 8-mL vial, was placed methyl 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoate (100.00 mg, 0.156 mmol, 1.00 equiv), NaOH (37.49 mg, 0.937 mmol, 6.00 equiv), MeOH (0.50 mL), dioxane (0.50 mL), H₂O (0.20 mL). The resulting solution was stirred for 14 hr at 70 degrees C. in an oil bath. The resulting mixture was concentrated. The resulting solution was diluted with 1 mL of H₂O. The pH value of the solution was adjusted to 6 with CH₃COOH. The resulting mixture was concentrated. The residue was applied onto a silica gel column and eluted with dichloromethane/methanol (10:1). This resulted in 60 mg of 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid as a brown solid. LC-MS-13: (ES, m/z):626.3 [M+H]⁺.

### Synthesis of 5-nitro-3-(oxan-4-yl)-3,4-dihydro-2H-1,4-benzoxazine-7-sulfonamide. Into a 50-mL round-bottom flask purged and maintained with an inert atmosphere of nitrogen, was placed 3-bromo-4-[[2-hydroxy-1-(oxan-4-yl)ethyl]amino]-5-nitrobenzenesulfonamide (1.40 g, 3.300 mmol, 1.00 equiv), dioxane (20.00 mL), Pd₂(dba)₃ (604.34 mg, 0.660 mmol, 0.20 equiv), XantPhos (763.73 mg, 1.320 mmol, 0.40 equiv), Cs₂CO₃ (3.23 g, 9.913 mmol, 3.00 equiv). The resulting solution was stirred for 2 hr at 100 degrees C. in an oil bath. The resulting mixture was concentrated. The residue was applied onto a silica gel column and eluted with ethyl acetate/petroleum ether (1:1). This resulted in 230 mg (20.30%) of 5-nitro-3-(oxan-4-yl)-3,4-dihydro-2H-1,4-benzoxazine-7-sulfonamide as a yellow solid. LC-MS-3: (ES, m/z):342.1 [M−H]⁻

Synthesis of (3S)-5-nitro-3-(oxan-4-yl)-3,4-dihydro-2H-1,4-benzoxazine-7-sulfonamide and (3R)-5-nitro-3-(oxan-4-yl)-3,4-dihydro-2H-1,4-benzoxazine-7-sulfonamide. Into a 50-mL round-bottom flask, was placed 5-nitro-3-(oxan-4-yl)-3,4-dihydro-2H-1,4-benzoxazine-7- sulfonamide (150.00 mg) and DMF(10 ml). The sample was purified by Chiral-Prep-HPLC with the following conditions (WATERS 2767): Column, CHIRALPAK IA, 250*20 mm, Sum; Mobile phase: A:n-Hexane:DMC=3:1, 0.1% DEA), B:EtOH; Gradient:10% B in 20 min; Detector, 220 nm. This resulted in 30 mg of (3S)-5-nitro-3-(oxan-4-yl)-3,4-dihydro-2H-1,4-benzoxazine-7-sulfonamide as a yellow solid. This resulted in 32 mg of (3R)-5-nitro-3-(oxan-4-yl)-3,4-dihydro-2H-1,4-benzoxazine-7-sulfonamide as a yellow solid.

Synthesis of 4-(4-[[2-(4-chlorophenyl)-4,4-dimethylcyclohex-1-en-1-yl]methyl]piperazin-1-yl)-2-[(12R) -12-methyl-13-oxa-2,4,10-triazatricyclo[7.4.0.0\hat{ }[3,7]]trideca-1(9),2,5,7-tetraen-10-yl]-N-[(3S)-5-nitro-3-(oxan-4-yl)-3,4-dihydro-2H-1,4-benzoxazin-7-ylsulfonyl]benzamide hydrochloride: Into a 8-mL vial, was placed (3S)-5-nitro-3-(oxan-4-yl)-3,4-dihydro-2H-1,4-benzoxazine-7- sulfonamide (20 mg, 0.058 mmol, 1.00 equiv), 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid (36.48 mg, 0.058 mmol, 1.00 equiv), DCM (0.5 mL), EDC.HCl (22.33 mg, 0.116 mmol, 2.00 equiv), DMAP (17.79 mg, 0.146 mmol, 2.50 equiv). The resulting solution was stirred for 14 hr at room temperature. The resulting mixture was concentrated. The residue was purified with Prep-TLC with dichloromethane/methanol (10:1). The crude product (30 mg) was purified by Prep-HPLC with the following conditions: Column, XBridge Prep C18 OBD 19*150 mm 5 um; Mobile phase, A: 0.1% HCl in water; B: ACN; Gradient: 38-58% B in 7 min; Flow rate: 20 ml/min; Detector, 220 nm. This resulted in 10 mg (17.38%) of 4-(4-[[2-(4-chlorophenyl)-4,4-dimethylcyclohex-1-en-1-yl]methyl]piperazin-1-yl)-2-[(12R) -12-methyl-13-oxa-2,4,10-triazatricyclo[7.4.0.0\hat{ }[3,7]]trideca-1(9),2,5,7-tetraen-10-yl]-N-[(3S)-5-nitro-3-(oxan-4-yl)-3,4-dihydro-2H-1,4-benzoxazin-7-ylsulfonyl]benzamide hydrochloride as a yellow solid.LC-MS-OA: (ES, m/z): 951.4 [M-HCl+H]f. ¹H NMR- OA (300 MHz, DMSO, ppm): δ12.15 (s, 1H), 10.86 (s, 1H), 9.68 (s, 1H), 8.78 (s, 1H), 8.05-7.81 (m, 1H), 7.58-7.31 (m, 1H), 7.21-6.72 (m, 6H), 6.70-6.40 (m, 1H), 6.02-5.81 (m, 1H), 4.61-4.11 (m, 3H), 3.92-3.78 (m, 4H), 3.65-3.61 (m, 2H), 3.38-3.13 (m, 8H), 2.90-2.72 (m, 2H), 2.30-2.21 (m, 2H), 2.13-1.94 (m, 2H), 1.92-1.51 (m, 3H), 1.51-1.23 (m, 8H), 0.96 (s, 6H).

Synthesis of 4-(4-[[2-(4-chlorophenyl)-4-methylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[(3R)-5-nitro-3-(oxan-4-yl)-3,4-dihydro-2H-1,4-benzoxazin-7-ylsulfonyl]benzamide hydrochloride. Into a 8-mL vial, was placed (3R)-5-nitro-3-(oxan-4-yl)-3,4-dihydro-2H-1,4-benzoxazine-7-sulfonami de (20.00 mg, 0.058 mmol, 1.00 equiv),4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid (36.48 mg, 0.058 mmol, 1.00 equiv), DCM, EDCI (22.33 mg, 0.116 mmol, 2.00 equiv), DMAP (17.79 mg, 0.146 mmol, 2.50 equiv). The resulting solution was stirred for 14 hr at room temperature. The resulting mixture was concentrated. The residue was purified with Prep-TLC with DCM/methanol (10:1). The crude product (30 mg) was purified by Prep-HPLC with the following conditions: Column, XBridge Prep C18 OBD 19*150 mm 5 um; Mobile phase, A: 0.1% HCl in water; B: ACN; Gradient: 38-58% B in 7 min; Flow rate: 20 ml/min; Detector, 220 nm. This resulted in 10.3 mg (18.16%) of 4-(4-[[2-(4-chlorophenyl)-4-methylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[(3R)-5-nitro-3-(oxan-4-yl)-3,4-dihydro-2H-1,4-benzoxazin-7-ylsulfonyl]benzamide hydrochloride as a yellow solid. LC-MS-0B: (ES, m/z): 951.4 [M-HCl+H]⁺ ¹H NMR- OB (300 MHz, DMSO, ppm): δ12.16 (s, 1H), 10.89 (s, 1H), 10.02 (s, 1H), 8.78 (s, 1H), 8.05-7.41 (m, 5H), 7.21-6.52 (m, 6H), 6.02-5.91 (m, 1H), 4.61-4.21 (m, 2H), 4.15-3.78 (m, 5H), 3.71-3.65 (m, 2H), 3.38-3.13 (m, 8H), 2.88-2.72 (m, 2H), 2.35-2.30 (m, 2H), 2.13-1.94 (m, 2H), 1.92-1.59 (m, 3H), 1.51-1.23 (m, 8H), 0.96 (s, 6H).

### Compound 2-14: Preparation of 4-(4-[[2-(4-chlorophenyl)-4,4-dimethylcyclohex-1-en-1-yl]methyl]piperazin-1-yl)-N-[(3S)-3-[(2S)-1,4-dioxan-2-yl]-5-nitro-3,4-dihydro-2H-1,4-benzoxazin-7-ylsulfonyl]-2-[(12R)-12-methyl-13-oxa-2,4,10-triazatricyclo[7.4.0.0\hat{ }[3,7]]trideca-1(9),2,5,7-tetraen-10-yl]benzamide hydrochloride, and 4-(4-[[2-(4-chlorophenyl)-4,4-dimethylcyclohex-1-en-1-yl]methyl]piperazin-1-yl)-N-[(3R)-3-[(2S)-1,4-dioxan-2-yl]-5-nitro-3,4-dihydro-2H-1,4-benzoxazin-7-ylsulfonyl]-2-[(12R)-12-methyl-13-oxa-2,4,10-triazatricyclo[7.4.0.0\hat{ }[3,7]]trideca-1(9),2,5,7-tetraen-10-yl]benzamide hydrochloride

Synthesis of (2R)-1,4-dioxane-2-carbaldehyde: Into a 100-mL round-bottom flask, was placed (2S)-1,4-dioxan-2-ylmethanol (2.36 g, 19.978 mmol, 1.00equiv), CH₃CN (50.00 mL, 1.218 mmol, 0.0 equiv), IBX (9.51 g, 33.962 mmol, 1.70equiv). The resulting solution was stirred for 4 h at 70 degrees C. in an oil bath. The solids were filtered out. The resulting mixture was concentrated. This resulted in 2.0 g (86.22%) of (2R)-1,4-dioxane-2-carbaldehyde as colorless oil. H-NMR-1: ¹H NMR (300 MHz, CDCl₃, ppm) δ9.66 (s, 1H), 4.12-3.77 (m, 7H).

Synthesis of 2-amino-2-[(2S)-1,4-dioxan-2-yl]acetonitrile: Into a 100-mL pressure tank reactor, was placed (2R)-1,4-dioxane-2-carbaldehyde (2.00 g, 17.224 mmol, 1.00equiv). NH₃/MeOH(7M) (40.00 mL). This was followed by the addition of TMSCN (2.97 g, 29.970 mmol, 1.74equiv). The resulting solution was stirred for overnight at 70 degrees C. in an oil bath. The resulting mixture was concentrated. The residue was applied onto a silica gel column and eluted with ethyl acetate/petroleum ether (3:1). The collected fractions were combined and concentrated. This resulted in 2.0 g (81.68%) of 2-amino-2-[(2S)-1,4-dioxan-2-yl]acetonitrile as brown oil. LCMS-2 (ES, m/z): M+1: 143.

Synthesis of amino((2S)-1,4-dioxan-2-yl)acetic acid: Into a 20-mL round-bottom flask, was placed 2-amino-2-[(2S)-1,4-dioxan-2-yl]acetonitrile (2.00 g, 14.069 mmol, 1.00equiv), NaOH(4M) (5.00 mL). The resulting solution was stirred for overnight at 70 degrees C. in an oil bath. The pH value of the solution was adjusted to 6 with HOAc. The solids were collected by filtration. The solid was dried in an oven under reduced pressure. This resulted in 1.2 g (52.93%) of amino((2S)-1,4-dioxan-2-yl)acetic acid as a white solid.LCMS-3 (ES, m/z): M+1: 162.

Synthesis of 2-amino-2-[(2S)-1,4-dioxan-2-yl]ethanol: Into a 100-mL round-bottom flask, was placed amino((2S)-1,4-dioxan-2-yl)acetic acid (1.20 g, 7.446 mmol, 1.00equiv), THE (40.00 mL, 0.555 mmol, 0.07equiv), LiAlH₄ (0.85 g, 22.396 mmol, 3.01equiv). The resulting solution was stirred for 4 h at 60 degrees C. in an oil bath. The reaction was then quenched by the addition of 2.4 g of Na₂SO₄.10H₂O. The solids were filtered out. The resulting mixture was concentrated. This resulted in 1.8 g (crude) of 2-amino-2-[(2S)-1,4-dioxan-2-yl]ethanol as colorless oil. LCMS-4 (ES, m/z): M+1: 148.

### Synthesis of 3-bromo-4-([1-[(2S)-1,4-dioxan-2-yl]-2-hydroxyethyl]amino)-5-nitrobenzenesulfonamide: Into a 50-mL round-bottom flask, was placed 3-bromo-4-chloro-5-nitrobenzenesulfonamide (1.19 g, 3.772 mmol, 1.00equiv), 2-amino-2-[(2S)-1,4-dioxan-2-yl]ethanol (0.72 g, 0.005 mmol, 1.3equiv), CH₃CN (30.00 mL), DIEA (1.46 g, 0.011 mmol, 3equiv). The resulting solution was stirred for 48 h at 80 degrees C. in an oil bath. The resulting mixture was concentrated. The residue was applied onto a silica gel column and eluted with ethyl acetate/petroleum ether (9:1). The collected fractions were combined and concentrated. This resulted in 120 mg (7.46%) of 3-bromo-4-([1-[(2S)-1,4-dioxan-2-yl]-2-hydroxyethyl]amino)-5-nitrobenzenesulfonamide as a yellow solid. LCMS-5 (ES, m/z): M-1: 424

Synthesis of (3S)-3-[(2S)-1,4-dioxan-2-yl]-5-nitro-3,4-dihydro-2H-1,4-benzoxazine-7-sulfonamide &(3R)-3-[(2S)-1,4-dioxan-2-yl]-5-nitro-3,4-dihydro-2H-1,4-benzoxazine-7-sulfonamide(assumed): Into a 8-mL round-bottom flask purged and maintained with an inert atmosphere of nitrogen, was placed 3-bromo-4-([1-[(2S)-1,4-dioxan-2-yl]-2-hydroxyethyl]amino)-5-nitrobenzenesulfonamide (120.00 mg, 0.282 mmol, 1.00equiv), dioxane (8.00 mL), Cs₂CO₃ (229.32 mg, 0.704 mmol, 2.50equiv), t-BuXPhos Pd G3 (22.33 mg, 0.028 mmol, 0.1 equiv). The resulting solution was stirred for 8 h at 100 degrees C. in an oil bath. The reaction was then quenched by the addition of water (8 mL). The resulting solution was extracted with 3×8 mL of ethyl acetate concentrated. The crude product was purified by Prep-HPLC with the following conditions: Column, X-bridge RP18; mobile phase, 0.05% ammonia in water and CH₃CN (45% CH₃CN up to 60% in 5 min); Detector, UV 254 nm. This resulted in 7 mg (7.20%) of (3S or 3R)-3-[(2S)-1,4-dioxan-2-yl]-5-nitro-3,4-dihydro-2H-1,4-benzoxazine-7-sulfonamide as a yellow solid. This resulted in 10 mg (10.29%) of (3R or 3S)-3-[(2S)-1,4-dioxan-2-yl]-5-nitro-3,4-dihydro-2H-1,4-benzoxazine-7-sulfonamide as a yellow solid. LC-MS-6: (ES, m/z): M-1:344.

### Synthesis of 4-(4-[[2-(4-chlorophenyl)-4,4-dimethylcyclohex-1-en-1-yl]methyl]piperazin-1-yl)-N-[(3S)-3-[(2S)-1,4-dioxan-2-yl]-5-nitro-3,4-dihydro-2H-1,4-benzoxazin-7-ylsulfonyl]-2-[(12R)-12-methyl-13-oxa-2,4,10-triazatricyclo[7.4.0.0\hat{ }[3,7]]trideca-1(9),2,5,7-tetraen-10-yl]benzamide hydrochloride(assumed): Into a 8-mL round-bottom flask, was placed 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid (12.69 mg, 0.020 mmol, 1.00equiv), (3S)-3-[(2S)-1,4-dioxan-2-yl]-5-nitro-3,4-dihydro-2H-1,4-benzoxazine-7-sulfonamide (7.00 mg, 0.020 mmol, 1.00equiv), DCM (5.00 mL), EDCI (7.77 mg, 0.041 mmol, 2.00equiv), DMAP (7.43 mg, 0.061 mmol, 3.00equiv). The resulting solution was stirred for overnight at room temperature. The resulting mixture was concentrated. The crude product was purified by Prep-HPLC with the following conditions (2 #SHIMADZU (HPLC-01)): Column, Sun Fire Prep C18 OBD Column, 19*150 mm Sum 10 nm; mobile phase, Water (0.05% TFA) and ACN (38% Phase B up to 58% in 7 min); Detector, UV 254 nm. The collected solution was concentrated under vacuum to remove CH₃CN and the resulting solution was dried by lyophilization (added with con.HCl (1 drop)). This resulted in 5 mg (24.92%) of 4-(4-[[2-(4-chlorophenyl)-4,4-dimethylcyclohex-1-en-1-yl]methyl]piperazin-1-yl)-N-[(3S)-3-[(2S)-1,4-dioxan-2-yl]-5-nitro-3,4-dihydro-2H-1,4-benzoxazin-7-ylsulfonyl]-2-[(12R)-12-methyl-13-oxa-2,4,10-triazatricyclo[7.4.0.0\hat{ }[3,7]]trideca-1(9),2,5,7-tetraen-10-yl]benzamide hydrochloride as a yellow solid. LC-MS-OA: (ES, m/z): M+1-HCl: 953

Synthesis of 4-(4-[[2-(4-chlorophenyl)-4,4-dimethylcyclohex-1-en-1-yl]methyl]piperazin-1-yl)-N-[(3R)-3-[(2S)-1,4-dioxan-2-yl]-5-nitro-3,4-dihydro-2H-1,4-benzoxazin-7-ylsulfonyl]-2-[(12R)-12-methyl-13-oxa-2,4,10-triazatricyclo[7.4.0.0\hat{ }[3,7]]trideca-1(9),2,5,7-tetraen-10-yl]benzamide hydrochloride(assumed): Into a 8-mL round-bottom flask, was placed (3R)-3-[(2S)-1,4-dioxan-2-yl]-5-nitro-3,4-dihydro-2H-1,4-benzoxazine-7-sulfonamide (10.00 mg, 0.029 mmol, 1.00equiv), 4-(4-[[2-(4-chlorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]benzoic acid (18.13 mg, 0.029 mmol, 1.00equiv), DCM (5.00 mL), EDCI (11.10 mg, 0.058 mmol, 2equiv), DMAP (14.15 mg, 0.116 mmol, 4equiv). The resulting solution was stirred for overnight at room temperature. The resulting mixture was concentrated. The crude product was purified by Prep-HPLC with the following conditions (2 #SHIMADZU (HPLC-01)): Column, Sun Fire Prep C18 OBD Column, 19??150 mm Sum 10 nm; mobile phase, Water (0.05% TFA) and ACN (38% Phase B up to 58% in 7 min); Detector, UV=254 nm. The collected solution was concentrated under vacuum to remove CH₃CN and the resulting solution was dried by lyophilization (added with con.HCl(1 drop)). This resulted in 10 mg of 4-(4-[[2-(4-chlorophenyl)-4,4-dimethylcyclohex-1-en-1-yl]methyl]piperazin-1-yl)-N-[(3R)-3-[(2S)-1,4-dioxan-2-yl]-5-nitro-3,4-dihydro-2H-1,4-benzoxazin-7-ylsulfonyl]-2-[(12R)-12-methyl-13-oxa-2,4,10-triazatricyclo[7.4.0.0\hat{ }[3,7]]trideca-1(9),2,5,7-tetraen-10-yl]benzamide hydrochloride as a yellow solid. LC-MS-OB: (ES, m/z): M+1-HCl: 953; H-NMR-OB: ¹H NMR (300 MHz, Methanol-d4, ppm) δ8.01 (s, 1H), 7.65 (d, J=8.4 Hz, 1H), 7.41 (d, J=8.4 Hz, 1H), 7.25 (s, 1H), 7.13 (d, J=8.4 Hz, 2H), 6.94 (s, 2H), 6.82 (s, 1H), 6.66 (s, 1H), 5.94 (s, 1H), 4.70 (s, 1H), 4.31 (s, 1H), 4.04 (s, 1H), 3.98-3.58 (m, 12H), 3.48 (s, 3H), 3.22 (s, 2H), 2.90 (s, 2H), 2.33 (s, 2H), 2.16 (s, 2H), 1.62 (t, J=6.3 Hz, 2H), 1.51 (d, J=6.3 Hz, 3H), 1.32 (s, 1H), 1.05 (s, 6H).

### Compound 2-15: Preparation of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(11S,15S)-16-oxa-2,4,10-triazatetracyclo[7.7.0.0\hat{ }[3,7]0.0\hat{ }[11,15]]hexadeca-1(9),2,5,7-tetraen-10-yl]benzamide(Assumed) and 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(11R,15R)-16-oxa-2,4,10-triazatetracyclo[7.7.0.0\hat{ }[3,7]0.0\hat{ }[11,15]]hexadeca-1(9),2,5,7-tetraen-10-yl]benzamide(Assumed)

Synthesis of N-[(trans)-2-hydroxycyclopentyl]-4-methylbenzenesulfonamide: Into a 250-mL round-bottom flask purged and maintained with an inert atmosphere of nitrogen, was placed (trans)-2-aminocyclopentan-1-ol (2.10 g, 20.761 mmol, 1.00 equiv), DCM (30.00 mL), TEA (3.18 g, 31.426 mmol, 1.51 equiv). This was followed by the addition of P-toluenesulfonyl chloride (4.35 g, 22.818 mmol, 1.10 equiv), in portions at 0 degrees C. The resulting solution was stirred for 3 hr at 25 degrees C. The resulting mixture was concentrated under vacuum. The residue was applied onto a silica gel column with ethyl acetate/petroleum ether (1.5:1). This resulted in 5 g (94.32%) of N-[(trans)-2-hydroxycyclopentyl]-4-methylbenzenesulfonamide as light yellow oil. ¹H NMR (300 MHz, Chloroform-d, ppm) δ7.84-7.77 (m, 2H), 7.38-7.30 (m, 2H), 5.27 (s, 1H), 4.06 (dt, J=7.2, 6.3 Hz, 1H), 3.25 (q, J=7.7 Hz, 1H), 2.91 (s, 1H), 2.45 (s, 3H), 2.06-1.81 (m, 2H), 1.72-1.46 (m, 3H), 1.45-1.23 (m, 1H).

Synthesis of N-[(trans)-2-[(5-bromo-1-[[2-(trimethylsilyl)ethoxy]methyl]pyrrolo[2,3-b]pyridin-6-yl)oxy]cyclopentyl]-4-methylbenzenesulfonamide: Into a 250-mL round-bottom flask purged and maintained with an inert atmosphere of nitrogen, was placed N-[(trans)-2-hydroxycyclopentyl]-4-methylbenzenesulfonamide (5.00 g, 19.583 mmol, 1.30 equiv), THE (50.00 mL). This was followed by the addition of NaH (1.81 g, 45.254 mmol, 3.01 equiv, 60%), in portions at 0 degrees C. The resulting solution was stirred for 0.5 h at 0 degrees C. To this was added 5-bromo-6-fluoro-1-[[2-(trimethylsilyl)ethoxy]methyl]pyrrolo[2,3-b]pyridine (5.19 g, 15.031 mmol, 1.00 equiv) at 0 degrees C. The resulting solution was stirred for 2 hr at 70 degrees C. in an oil bath. The reaction mixture was cooled to room temperature. The reaction was then quenched by the addition of aqueous NH₄Cl. The resulting solution was extracted with 3×200 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:3). This resulted in 4.8 g (55.00%) of N-[(trans)-2-[(5-bromo-1-[[2-(trimethylsilyl)ethoxy]methyl]pyrrolo[2,3-b]pyridin-6-yl)oxy]cyclopentyl]-4-methylbenzenesulfonamide as yellow oil. ¹H NMR (300 MHz, Chloroform-d, ppm) δ8.00 (s, 1H), 7.56-7.47 (m, 2H), 7.19 (d, J=3.6 Hz, 1H), 7.11-7.02 (m, 2H), 6.42 (d, J=3.6 Hz, 1H), 5.70 (d, J=10.9 Hz, 1H), 5.49 (d, J=10.9 Hz, 1H), 5.33-5.20 (m, 1H), 3.69-3.43 (m, 3H), 2.31 (s, 3H), 1.93-1.63 (m, 4H), 1.01-0.93 (m, 2H), 0.89 (dd, J=7.0, 1.5 Hz, 2H), 0.01 (s, 9H).

Synthesis of (trans)-10-(4-methylbenzenesulfonyl)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene: Into a 100-mL round-bottom flask purged and maintained with an inert atmosphere of nitrogen, was placed N-[(trans)-2-[(5-bromo-1-[[2-(trimethylsilyl)ethoxy]methyl]pyrrolo[2,3-b]pyridin-6-yl)oxy]cyclopentyl]-4-methylbenzenesulfonamide (2.90 g, 4.995 mmol, 1.00 equiv), DMF (30.00 mL), phen (902.00 mg, 5.005 mmol, 1.00 equiv), CuI (952.00 mg, 4.999 mmol, 1.00 equiv), K₂CO₃ (2.07 g, 14.978 mmol, 3.00 equiv). The resulting solution was stirred overnight at 120 degrees C. in an oil bath. The reaction mixture was cooled to room temperature. The resulting solution was diluted with 500 mL of water. The resulting solution was extracted with 3×200 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:3). This resulted in 2.1 g (84.14%) of (trans)-10-(4-methylbenzenesulfonyl)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene as colorless oil. ¹H NMR (300 MHz, Chloroform-d, ppm) δ8.68 (s, 1H), 7.43-7.34 (m, 2H), 7.24 (d, J=3.6 Hz, 1H), 7.15 (d, J=8.0 Hz, 2H), 6.53 (d, J=3.6 Hz, 1H), 5.54 (d, J=1.4 Hz, 2H), 4.00 (td, J=10.0, 7.6 Hz, 1H), 3.52 (t, J=8.2 Hz, 2H), 3.43-3.28 (m, 1H), 2.68-2.49 (m, 1H), 2.35 (s, 3H), 2.21-1.88 (m, 4H), 1.77 (td, J=10.8, 7.6 Hz, 1H), 0.97-0.86 (m, 2H), -0.05 (s, 9H).

Synthesis of (11S,15S)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene(Assumed) and (11R,15R)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene(Assumed): Into a 100-mL round-bottom flask, was placed Mg (1.92 g, 78.996 mmol, 19.74 equiv), MeOH (20.00 mL), (trans)-10-(4-methylbenzenesulfonyl)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene (2.00 g, 4.002 mmol, 1.00 equiv). The resulting solution was stirred for 2 hr at 60 degrees C. in an oil bath. The reaction mixture was cooled to room temperature. The resulting solution was diluted with 300/300 mL of NaHCO₃and CH₂Cl₂. The solids were filtered out and the organic was separated. The mixture was dried over anhydrous sodium sulfate and concentrated under vacuum. The residue was applied onto a silica gel column with ethyl acetate/petroleum ether (1:3). The crude product was purified by Chiral-Prep-HPLC with the following conditions: Mobile phase: A: n-Hexane B: ETOH; Flow rate: 20 mL/min; Column: DAICEL CHIRALPAK OD, 250*20 mm, 5 um; Gradient: 20% B in 15 min; 220 nm. This resulted in 360 mg (26.03%) of (11S,15S)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene(Assumed) as yellow oil. This resulted in 400 mg (28.92%) of (11R,15R)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene(Assumed) as yellow oil. Peak 1: LC-MS (ES, m/z): M+1=346, ¹H NMR (300 MHz, Chloroform-d, ppm) δ7.28 (s, 1H), 7.15 (d, J=3.5 Hz, 1H), 6.33 (d, J=3.5 Hz, 1H), 5.56 (s, 2H), 4.30-4.07 (m, 1H), 3.55 (dd, J=8.8, 7.5 Hz, 2H), 3.29 (dt, J=11.4, 7.5 Hz, 1H), 2.37-2.19 (m, 1H), 2.18-2.04 (m, 1H), 2.04-1.79 (m, 3H), 1.68-1.40 (m, 1H), 0.90 (dd, J=8.7, 7.5 Hz, 2H), -0.06 (s, 9H). Peak 2: LC-MS (ES, m/z): M+1=346, ¹H NMR (300 MHz, Chloroform-d, ppm) δ7.28 (s, 1H), 7.15 (d, J=3.5 Hz, 1H), 6.33 (d, J=3.5 Hz, 1H), 5.56 (s, 2H), 4.30-4.07 (m, 1H), 3.55 (dd, J=8.8, 7.5 Hz, 2H), 3.29 (dt, J=11.4, 7.5 Hz, 1H), 2.37-2.19 (m, 1H), 2.18-2.04 (m, 1H), 2.04-1.79 (m, 3H), 1.68-1.40 (m, 1H), 0.90 (dd, J=8.7, 7.5 Hz, 2H), -0.06 (s, 9H).

Synthesis of methyl 4-(4-[[2-(4-chlorophenyl)-4,4-dimethylcyclohex-1-en-1-yl]methyl]piperazin-1-yl)-2-[(11S,15S)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed): Into a 40-mL vial purged and maintained with an inert atmosphere of nitrogen, was placed (11S,15S)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene(Assumed) (340.00 mg, 0.984 mmol, 1.00 equiv), toluene (15.00 mL), methyl 2-bromo-4-(4-[[2-(4-chlorophenyl)-4,4-dimethylcyclohex-1-en-1-yl]methyl]piperazin-1-yl)benzoate (1.04 g, 1.955 mmol, 1.99 equiv), Pd₂(dba)₃.CHCl₃ (204.00 mg, 0.197 mmol, 0.20 equiv), Xantphos (228.00 mg, 0.394 mmol, 0.40 equiv), Cs₂CO₃ (961.00 mg, 2.949 mmol, 3.00 equiv). The resulting solution was stirred for 4 hr at 100 degrees C. in an oil bath. The reaction mixture was cooled to room temperature. The resulting mixture was concentrated under vacuum. The residue was applied onto a silica gel column with ethyl acetate/petroleum ether (1:2). This resulted in 630 mg (80.38%) of methyl 4-(4-[[2-(4-chlorophenyl)-4,4-dimethylcyclohex-1-en-1-yl]methyl]piperazin-1-yl)-2-[(11 S,15S)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) as a yellow solid. LC-MS (ES, m/z): M+1=796.

Synthesis of methyl 4-(4-[[2-(4-chlorophenyl)-4,4-dimethylcyclohex-1-en-1-yl]methyl]piperazin-1-yl)-2-[(11S,15S)-16-oxa-2,4,10-triazatetracyclo[7.7.0.0\hat{ }[3,7]0.0\hat{ }11,15]]hexadeca-1(9),2,5,7-tetraen-10-yl]benzoate(Assumed): Into a 40-mL vial, was placed methyl 4-(4-[[2-(4-chlorophenyl)-4,4-dimethylcyclohex-1-en-1-yl]methyl]piperazin-1-yl)-2-[(11S,15S)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) (630.00 mg, 0.791 mmol, 1.00 equiv), TBAF in THE (10.00 mL, 1.0 M), ethylenediamine (1.30 g, 21.631 mmol, 27.35 equiv). The resulting solution was stirred for 8 hr at 70 degrees C. in an oil bath. The reaction mixture was cooled to room temperature. The resulting mixture was concentrated under vacuum. The residue was applied onto a silica gel column with ethyl acetate/petroleum ether (2:1). This resulted in 300 mg (56.93%) of methyl 4-(4-[[2-(4-chlorophenyl)-4,4-dimethylcyclohex-1-en-1-yl]methyl]piperazin-1-yl)-2-[(11 S,15S)-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) as a yellow solid. LC-MS: (ES, m/z): M+1=666.

Synthesis of 4-(4-[[2-(4-chlorophenyl)-4,4-dimethylcyclohex-1-en-1-yl]methyl]piperazin-1-yl)-2-[(11S,15S)-16-oxa-2,4,10-triazatetracyclo[7.7.0.0\hat{ }[3,7]0.0\hat{ }11,15]]hexadeca-1(9),2,5,7-tetraen-10-yl]benzoic acid(Assumed): Into a 40-mL vial, was placed methyl 4-(4-[[2-(4-chlorophenyl)-4,4-dimethylcyclohex-1-en-1-yl]methyl]piperazin--yl)-2-[(11S,15S)-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) (300.00 mg, 0.450 mmol, 1.00 equiv), dioxane (5.00 mL), MeOH (5.00 mL), NaOH (1.00 mL, 4.000 mmol, 8.88 equiv). The resulting solution was stirred for 4 hr at 70 degrees C. in an oil bath. The reaction mixture was cooled to room temperature. The resulting mixture was concentrated under vacuum. The pH value of the solution was adjusted to 6-7 with HCl (2 mol/L). The resulting solution was extracted with 3×50 mL of ethyl acetate and the organic layers combined. The resulting mixture was washed with 1×300 ml of brine. The mixture was dried over anhydrous sodium sulfate and concentrated under vacuum. The residue was purified by Prep-TLC with ethyl acetate. This resulted in 130 mg (44.26%) of 4-(4-[[2-(4-chlorophenyl)-4,4-dimethylcyclohex-1-en-1-yl]methyl]piperazin-1-yl)-2-[(11 S,15S)-16-oxa-2,4,10-triazatetracyclo[7.7.0.0\hat{ }[3,7]0.0\hat{ }[11,15]]hexadeca-1(9),2,5,7-tetraen-10-yl]benzoic acid(Assumed) as a white solid. LC-MS: (ES, m/z): M+1=652.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(11S,15S or 11R, 15R)-16-oxa-2,4,10-triazatetracyclo[7.7.0.0\hat{ }[3,7]0.0\hat{ }11,15]]hexadeca-1(9),2,5,7-tetraen-10-yl]benzamide(Assumed): Into a 40-mL vial, was placed 4-(4-[[2-(4-chlorophenyl)-4,4-dimethylcyclohex-1-en-1-yl]methyl]piperazin-1-yl)-2-[(11S,15S)-16-oxa-2,4,10-triazatetracyclo[7.7.0.0\hat{ }[3,7]0.0\hat{ }[11,15]]hexadeca-1(9),2,5,7-tetraen-10-yl]benzoic acid(Assumed) (50.00 mg, 0.077 mmol, 1.00 equiv), DCM (5.00 mL), 4-[[(2S)-1,4-dioxan-2-ylmethyl]amino]-3-nitrobenzenesulfonamide (24.00 mg, 0.076 mmol, 0.99 equiv), EDCI (30.00 mg, 0.156 mmol, 2.04 equiv), DMAP (38.00 mg, 0.311 mmol, 4.06 equiv). The resulting solution was stirred overnight at 30 degrees C. in an oil bath. The resulting mixture was concentrated under vacuum. The crude product was purified by Prep-HPLC with the following conditions (Prep-HPLC-006): Column, X Bridge Shield RP18 OBD Column, Sum, 19*150 mm; mobile phase, Water (0.05% NH₃.H₂O) and ACN (40% Phase B up to 70% in 7 min); Detector, UV 254/220 nm. This resulted in 25 mg (34.27%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(11S,15S or 11R, 15R)-16-oxa-2,4,10-triazatetracyclo[7.7.0.0\hat{ }[3,7]0.0\hat{ }[11,15]]hexadeca-1(9),2,5,7-tetraen-10-yl]benzamide(Assumed) as a yellow solid. LC-MS (ES, m/z): M+1=951, ¹H NMR (300 MHz, DMSO-d₆, ppm) δ12.90 (s, 1H), 11.21 (s, 1H), 8.54 (s, 1H), 8.35 (d, J=2.3 Hz, 1H), 7.75 (d, J=8.6 Hz, 1H), 7.46-7.30 (m, 3H), 7.18-6.99 (m, 3H), 6.93 (d, J=9.2 Hz, 1H), 6.80 (s, 2H), 6.49 (s, 1H), 6.07-5.99 (m, 1H), 4.33 (d, J=7.2 Hz, 1H), 3.79 (dd, J=9.9, 5.9 Hz, 4H), 3.73-3.33 (m, 7H), 3.27 (d, J=13.6 Hz, 4H), 3.12 (d, J=6.5 Hz, 1H), 2.75 (s, 2H), 2.20 (s, 6H), 1.97 (s, 2H), 1.83 (s, 2H), 1.68 (s, 1H), 1.40 (s, 3H), 0.94 (d, J=2.2 Hz, 6H).

Synthesis of methyl 4-(4-[[2-(4-chlorophenyl)-4,4-dimethylcyclohex-1-en-1-yl]methyl]piperazin-1-yl)-2-[(11R,15R)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed): Into a 40-mL vial purged and maintained with an inert atmosphere of nitrogen, was placed (11R,15R)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraene(Assumed) (400.00 mg, 1.158 mmol, 1.00 equiv), toluene (15 mL), methyl 2-bromo-4-(4-[[2-(4-chlorophenyl)-4,4-dimethylcyclohex-1-en-1-yl]methyl]piperazin-1-yl)benzoate (1.23 g, 2.312 mmol, 2.00 equiv), Pd₂(dba)₃.CHCl₃ (239.00 mg, 0.231 mmol, 0.20 equiv), Xantphos (268.00 mg, 0.463 mmol, 0.40 equiv), Cs₂CO₃ (1.13 g, 3.468 mmol, 3.00 equiv). The resulting solution was stirred for 4 hr at 100 degrees C. in an oil bath. The reaction mixture was cooled to room temperature. The resulting mixture was concentrated under vacuum. The residue was applied onto a silica gel column with ethyl acetate/petroleum ether (1:2). This resulted in 680 mg (73.74%) of methyl 4-(4-[[2-(4-chlorophenyl)-4,4-dimethylcyclohex-1-en-1-yl]methyl]piperazin-1-yl)-2-[(11R,15R)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) as a yellow solid. LC-MS (ES, m/z): M+1=796.

Synthesis of methyl 4-(4-[[2-(4-chlorophenyl)-4,4-dimethylcyclohex-1-en-1-yl]methyl]piperazin-1-yl)-2-[(11R,15R)-16-oxa-2,4,10-triazatetracyclo[7.7.0.0\hat{ }[3,7]0.0\hat{ }11,15]]hexadeca-1(9),2,5,7-tetraen-10-yl]benzoate(Assumed): Into a 40-mL vial, was placed methyl 4-(4-[[2-(4-chlorophenyl)-4,4-dimethylcyclohex-1-en-1-yl]methyl]piperazin-1-yl)-2-[(11R,15R)-4-[[2-(trimethylsilyl)ethoxy]methyl]-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) (670.00 mg, 0.841 mmol, 1.00 equiv), TBAF in THE (10.00 mL, 1.0 M), ethylenediamine (1.30 g, 21.631 mmol, 25.72 equiv). The resulting solution was stirred for 8 hr at 70 degrees C. in an oil bath. The reaction mixture was cooled to room temperature. The resulting mixture was concentrated under vacuum. The residue was applied onto a silica gel column with ethyl acetate/petroleum ether (2:1). This resulted in 320 mg (57.10%) of methyl 4-(4-[[2-(4-chlorophenyl)-4,4-dimethylcyclohex-1-en-1-yl]methyl]piperazin-1-yl)-2-[(11R,15R)-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) as a yellow solid. LC-MS (ES, m/z): M+1=666.

Synthesis of 4-(4-[[2-(4-chlorophenyl)-4,4-dimethylcyclohex-1-en-1-yl]methyl]piperazin-1-yl)-2-[(11R,15R)-16-oxa-2,4,10-triazatetracyclo[7.7.0.0\hat{ }[3,7]0.0\hat{ }11,15]]hexadeca-1(9),2,5,7-tetraen-10-yl]benzoic acid(Assumed): Into a 40-mL vial, was placed methyl 4-(4-[[2-(4-chlorophenyl)-4,4-dimethylcyclohex-1-en-1-yl]methyl]piperazin-1-yl)-2-[(11R,15R)-16-oxa-2,4,10-triazatetracyclo[7.7.0.0\hat{ }[3,7]0.0\hat{ }[11,15]]hexadeca-1(9),2,5,7-tetraen-10-yl]benzoate(Assumed) (140.00 mg, 0.210 mmol, 1.00 equiv), dioxane (3.00 mL), MeOH (3.00 mL), NaOH (0.60 mL, 2.400 mmol, 11.42 equiv). The resulting solution was stirred for 4 hr at 70 degrees C. in an oil bath. The reaction mixture was cooled to room temperature. The resulting mixture was concentrated under vacuum. The pH value of the solution was adjusted to 6-7 with HCl (2 mol/L). The resulting solution was extracted with 3×50 mL of ethyl acetate and the organic layers combined. The resulting mixture was washed with 1×300 ml of brine. The mixture was dried over anhydrous sodium sulfate and concentrated under vacuum. The residue was purified by Prep-TLC with ethyl acetate. This resulted in 80 mg (58.37%) of 4-(4-[[2-(4-chlorophenyl)-4,4-dimethylcyclohex-1-en-1-yl]methyl]piperazin-1-yl)-2-[(11R,15R)-16-oxa-2,4,10-triazatetracyclo[7.7.0.0\hat{ }[3,7]0.0\hat{ }[11,15]]hexadeca-1(9),2,5,7-tetraen-10-yl]benzoic acid(Assumed) as a white solid. LC-MS (ES, m/z): M+1=652.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(11R,15R or 11S, 15S)-16-oxa-2,4,10-triazatetracyclo[7.7.0.0\hat{ }[3,7]0.0\hat{ }11,15]]hexadeca-1(9),2,5,7-tetraen-10-yl]benzamide: Into a 40-mL vial, was placed 4-(4-[[2-(4-chlorophenyl)-4,4-dimethylcyclohex-1-en-1-yl]methyl]piperazin-1-yl)-2-[(11R,15R)-16-oxa-2,4,10-triazatetracyclo[7.7.0.0\hat{ }[3,7]0.0\hat{ }[11,15]]hexadeca-1(9),2,5,7-tetraen-10-yl]benzoic acid (20.00 mg, 0.031 mmol, 1.00 equiv), DCM (5.00 mL), 4-[[(2S)-1,4-dioxan-2-ylmethyl]amino]-3-nitrobenzenesulfonamide(Assumed) (9.70 mg, 0.031 mmol, 1.00 equiv), EDCI (12.00 mg, 0.063 mmol, 2.04 equiv), DMAP (15.00 mg, 0.123 mmol, 4.00 equiv).

The resulting solution was stirred overnight at 30 degrees C. in an oil bath. The resulting mixture was concentrated under vacuum. The crude product was purified by Prep-HPLC with the following conditions (Prep-HPLC-006): Column, X Bridge Shield RP18 OBD Column, Sum, 19*150 mm; mobile phase, Water (0.05% NH₃.H₂O) and ACN (40% Phase B up to 70% in 7 min); Detector, UV 254/220 nm. This resulted in 4.7 mg (16.11%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(11R,15R or 11S, 15S)-16-oxa-2,4,10-triazatetracyclo[7.7.0.0\hat{ }[3,7]0.0\hat{ }[11,15]]hexadeca-1(9),2,5,7-tetraen-10-yl]benzamide(Assumed) as a yellow solid. LC-MS (ES, m/z): M+1=951, ¹H NMR (300 MHz, DMSO-d₆, ppm) δ12.90 (s, 1H), 11.21 (s, 1H), 8.54 (s, 1H), 8.35 (d, J=2.3 Hz, 1H), 7.75 (d, J=8.6 Hz, 1H), 7.46-7.30 (m, 3H), 7.18-6.99 (m, 3H), 6.93 (d, J=9.2 Hz, 1H), 6.80 (s, 2H), 6.49 (s, 1H), 6.07-5.99 (m, 1H), 4.33 (d, J=7.2 Hz, 1H), 3.79 (dd, J=9.9, 5.9 Hz, 4H), 3.73-3.33 (m, 7H), 3.27 (d, J=13.6 Hz, 4H), 3.12 (d, J=6.5 Hz, 1H), 2.75 (s, 2H), 2.20 (s, 6H), 1.97 (s, 2H), 1.83 (s, 2H), 1.68 (s, 1H), 1.40 (s, 3H), 0.94 (d, J=2.2 Hz, 6H).

### Compound 2-16: Preparation of 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]-N-(4-[[(2S)-1,4-dioxan-2-ylmethyl]amino]-3-nitrobenzenesulfonyl)benzamide (assumed) and 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7].0\hat{ }[11,15]]hexadeca-1(9),2,5,7-tetraen-10-yl]-N-(4-[[(2S)-1,4-dioxan-2-ylmethyl]amino]-3-nitrobenzenesulfonyl)benzamide (assumed)

Synthesis of N-[(trans)-4-hydroxyoxolan-3-yl]-4-methylbenzenesulfonamide: Into a 250-mL round-bottom flask purged and maintained with an inert atmosphere of nitrogen, was placed 3,6-dioxabicyclo[3.1.0]hexane (5.00 g, 58.079 mmol, 1.00 equiv), dioxane (100.00 mL), p-toluenesulfonamide (11.93 g, 69.681 mmol, 1.20 equiv), TEBAC (1.33 g, 5.833 mmol, 0.10 equiv), K₂CO₃ (0.80 g, 5.788 mmol, 0.10 equiv). The resulting solution was stirred for 3 days at 90 degrees C. in an oil bath. The reaction mixture was cooled to room temperature. The solids were filtered out. The resulting mixture was concentrated under vacuum. The residue was applied onto a silica gel column with ethyl acetate/petroleum ether (2:1). This resulted in 10.69 g (25.75%) of N-[(trans) -4-hydroxyoxolan-3-yl]-4-methylbenzenesulfonamide as a white solid. LC-MS: (ES, m/z): M+1=258.

Synthesis of N-[(trans) -4-[(5-bromo-1-[[2-(trimethylsilyl)ethoxy]methyl]pyrrolo[2,3-b]pyridin-6-yl)oxy]oxolan-3-yl]-4-methylbenzenesulfonamide: Into a 250-mL round-bottom flask purged and maintained with an inert atmosphere of nitrogen, was placed N-[(trans)-4-hydroxyoxolan-3-yl]-4-methylbenzenesulfonamide (10.50 g, 14.691 mmol, 1.30 equiv, 36%), THE (100 mL). This was followed by the addition of NaH (2.71 g, 67.756 mmol, 6.01 equiv, 60%), in portions at 0 degrees C. The resulting solution was stirred for 30 min at 0 degrees C. To this was added 5-bromo-6-fluoro-1-[[2-(trimethylsilyl)ethoxy]methyl]pyrrolo[2,3-b]pyridine (3.89 g, 11.266 mmol, 1.00 equiv) at 0 degrees C. The resulting solution was stirred for 4 hr at 70 degrees C. in an oil bath. The reaction mixture was cooled to room temperature. The reaction was then quenched by the addition of 500 mL of aqueous NH₄Cl. The resulting solution was extracted with 3×300 mL of ethyl acetate and the organic layers combined. The resulting mixture was washed with 1×1500 mL of brine. The mixture was dried over anhydrous sodium sulfate and concentrated under vacuum. The residue was applied onto a silica gel column with ethyl acetate/petroleum ether (1:2). This resulted in 3 g (45.71%) of N-[(trans) -4-[(5-bromo-1-[[2-(trimethylsilyl)ethoxy]methyl]pyrrolo[2,3-b]pyridin-6-yl)oxy]oxolan-3-yl]-4-methylbenzenesulfonamide as light yellow oil. ¹H NMR (300 MHz, Chloroform-d, ppm) δ8.04 (s, 1H), 7.78-7.61 (m, 2H), 7.25-7.10 (m, 3H), 6.43 (d, J=3.6 Hz, 1H), 5.67 (d, J=10.8 Hz, 1H), 5.60 (d, J=5.7 Hz, 1H), 5.49 (d, J=10.8 Hz, 1H), 5.40 (dt, J=6.1, 3.1 Hz, 1H), 4.27 (dd, J=10.5, 5.9 Hz, 1H), 4.21-4.03 (m, 1H), 4.03-3.86 (m, 2H), 3.70-3.52 (m, 3H), 2.32 (s, 3H), 0.93 (ddt, J=10.6, 5.5, 2.6 Hz, 2H), -0.02 (s, 9H).

Synthesis of (trans) -10-(4-methylbenzenesulfonyl)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene: Into a 250-mL round-bottom flask purged and maintained with an inert atmosphere of nitrogen, was placed N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide (3.00 g, 5.149 mmol, 1.00 equiv), DMF (50.00 mL), phen (743.00 mg, 4.123 mmol, 0.80 equiv), CuI (785.00 mg, 4.122 mmol, 0.80 equiv), K₂CO₃ (2.14 g, 15.484 mmol, 3.01 equiv). The resulting solution was stirred for 2 days at 120 degrees C. in an oil bath. The reaction mixture was cooled to room temperature.

The resulting solution was diluted with 500 mL of water. The resulting solution was extracted with 3×200 mL of ethyl acetate and the organic layers combined. The resulting mixture was washed with 1×1000 mL of brine. The mixture was dried over anhydrous sodium sulfate and concentrated under vacuum. The residue was applied onto a silica gel column with ethyl acetate/petroleum ether (1:2). This resulted in 2.5 g (82.26%) of (trans) -10-(4-methylbenzenesulfonyl)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene as yellow oil. LC-MS: (ES, m/z) M+1=502.

Synthesis of (11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) and (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed): Into a 100-mL round-bottom flask, was placed Mg (2.04 g, 83.933 mmol, 19.81 equiv), MeOH (30.00 mL), (trans) -10-(4-methylbenzenesulfonyl)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (2.50 g, 4.236 mmol, 1.00 equiv, 85%). The resulting solution was stirred for 2 hr at 60 degrees C. in an oil bath. The reaction mixture was cooled to room temperature. The resulting solution was diluted with 300/300 mL of NaHCO₃and CH₂Cl₂. The solids were filtered out and the organic was separated. The mixture was dried over anhydrous sodium sulfate and concentrated under vacuum. The residue was applied onto a silica gel column with ethyl acetate/petroleum ether (1:3). The crude product was purified by Chiral-Prep-HPLC with the following conditions: Mobile phase: A:n-Hexane (0.1% DEA) B:ETOH; Flow rate: 20 mL/min; Column: DAICEL CHIRALPAK IA, 250*20 mm, Sum; Gradient:12% B in 20 min; 220 nm. This resulted in 350 mg (23.78%) of (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) as yellow oil. and 400 mg (27.18%) of (11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) as yellow oil. Peak 1: LC-MS: (ES, m/z) M+1=348. ¹H NMR (300 MHz, Chloroform-d, ppm) δ7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H), -0.04 (s, 9H). Peak 2: LC-MS: (ES, m/z) M+1=348. ¹H NMR (300 MHz, Chloroform-d, ppm) δ7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H), -0.04 (s, 9H).

Synthesis of methyl 4-(4-[[2-(4-chlorophenyl)-4,4-dimethylcyclohex-1-en-1-yl]methyl]piperazin-1-yl)-2-[(11R,15S)-4-[[2-(trimethylsilyl)ethoxy]methyl]-13,16-dioxa-2,4,10-triazatetracyclo[7.7.0.0\hat{ }[3,7]0.0\hat{ }11,15]]hexadeca-1(9),2,5,7-tetraen-10-yl]benzoate (assumed): Into a 40-mL vial purged and maintained with an inert atmosphere of nitrogen, was placed (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) (350.00 mg, 1.007 mmol, 1.00 equiv), toluene (15.00 mL), methyl 2-bromo-4-(4-[[2-(4-chlorophenyl)-4,4-dimethylcyclohex-1-en-1-yl]methyl]piperazin-1-yl)benzoate (1.07 g, 2.012 mmol, 2.00 equiv), Pd₂(dba)₃.CHCl₃ (208.00 mg, 0.201 mmol, 0.20 equiv), Xantphos (234.00 mg, 0.404 mmol, 0.40 equiv), Cs₂CO₃ (985.00 mg, 3.023 mmol, 3.00 equiv). The resulting solution was stirred for 3 hr at 100 degrees C. in an oil bath. The reaction mixture was cooled to room temperature. The resulting mixture was concentrated under vacuum. The residue was applied onto a silica gel column with ethyl acetate/petroleum ether (1:2). This resulted in 720 mg (89.52%) of methyl 4-(4-[[2-(4-chlorophenyl)-4,4-dimethylcyclohex-1-en-1-yl]methyl]piperazin-1-yl)-2-[(11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) as a yellow solid. LC-MS: (ES, m/z) M+1=798.

Synthesis of methyl 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }11,15]]hexadeca-1(9),2,5,7-tetraen-10-yl]benzoate (assumed): Into a 40-mL vial, was placed methyl 4-(4-[[2-(4-chlorophenyl)-4,4-dimethylcyclohex-1-en-1-yl]methyl]piperazin-1-yl)-2-[(11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) (720.00 mg, 0.902 mmol, 1.00 equiv), 1.0 M TBAF/THF (15.00 mL), ethylenediamine (1.30 g, 21.631 mmol, 23.99 equiv). The resulting solution was stirred for 8 hr at 70 degrees C. in an oil bath. The reaction mixture was cooled to room temperature.

The resulting mixture was concentrated under vacuum. The residue was applied onto a silica gel column with ethyl acetate/petroleum ether (2:1). This resulted in 350 mg (58.09%) of methyl 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) as a light yellow solid. LC-MS: (ES, m/z) M+1=668.

Synthesis of 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed): Into a 40-mL vial, was placed methyl 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) (150.00 mg, 0.224 mmol, 1.00 equiv), dioxane (3.00 mL), MeOH (3.00 mL), NaOH (0.60 mL, 2.400 mmol, 10.69 equiv). The resulting solution was stirred for 4 hr at 70 degrees C. in an oil bath. The reaction mixture was cooled to room temperature. The resulting mixture was concentrated under vacuum. The pH value of the solution was adjusted to 5-6 with HCl (2 mol/L). The resulting solution was extracted with 3×50 mL of ethyl acetate and the organic layers combined. The resulting mixture was washed with 1×300 mL of brine. The mixture was dried over anhydrous sodium sulfate and concentrated under vacuum. The residue was purified by Prep-TLC with dichloromethane/methanol (10:1). This resulted in 80 mg (54.48%) of 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed) as a white solid. LC-MS: (ES, m/z) M+1=654.

Synthesis of 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }11,15]]hexadeca-1(9),2,5,7-tetraen-10-yl]-N-(4-[[(2S)-1,4-dioxan-2-ylmethyl]amino]-3-nitrobenzenesulfonyl)benzamide (assumed): Into a 40-mL vial, was placed 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed) (50.00 mg, 0.076 mmol, 1.00 equiv), DCM (5.00 mL), 4-[[(2S)-1,4-dioxan-2-ylmethyl]amino]-3-nitrobenzenesulfonamide (24.00 mg, 0.076 mmol, 0.99 equiv), EDCI (29.00 mg, 0.151 mmol, 1.98 equiv), DMAP (37.00 mg, 0.303 mmol, 3.96 equiv). The resulting solution was stirred overnight at 30 degrees C. in an oil bath. The resulting mixture was concentrated under vacuum. The crude product was purified by Prep-HPLC with the following conditions (Prep-HPLC-006): Column, XBridge Shield RP18 OBD Column, Sum, 19*150 mm; mobile phase, Water (0.05% NH₃.H₂O) and ACN (40% Phase B up to 70% in 7 min); Detector, UV 254/220 nm. This resulted in 25 mg (34.31%) of 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7].0\hat{ }[11,15]]hexadeca-1(9),2,5,7-tetraen-10-yl]-N-(4-[[(2S)-1,4-dioxan-2-ylmethyl]amino]-3-nitrobenzenesulfonyl)benzamide (assumed) as a yellow solid. LC-MS: (ES, m/z) M+1=953. ¹H NMR (300 MHz, DMSO-d₆, ppm) δ12.27 (s, 1H), 11.05 (d, J=66.1 Hz, 1H), 8.42 (d, J=30.6 Hz, 1H), 8.32 (d, J=2.3 Hz, 1H), 7.57 (s, 1H), 7.36 (dd, J=8.4, 4.1 Hz, 3H), 7.08 (ddd, J=14.1, 5.9, 2.6 Hz, 3H), 6.78 (t, J=29.3 Hz, 3H), 6.57 (s, 1H), 6.06-5.94 (m, 1H), 4.45 (dd, J=98.7, 9.1 Hz, 3H), 3.96-3.34 (m, 12H), 3.19 (d, J=16.7 Hz, 4H), 2.81-2.71 (m, 2H), 2.24 (d, J=19.4 Hz, 6H), 1.98 (s, 2H), 1.41 (s, 2H), 0.94 (s, 6H).

Synthesis of methyl 4-(4-[[2-(4-chlorophenyl)-4,4-dimethylcyclohex-1-en-1-yl]methyl]piperazin-1-yl)-2-[(11S,15R)-4-[[2-(trimethylsilyl)ethoxy]methyl]-13,16-dioxa-2,4,10-triazatetracyclo[7.7.0.0\hat{ }[3,7]0.0\hat{ }11,15]]hexadeca-1(9),2,5,7-tetraen-10-yl]benzoate (assumed): Into a 40-mL vial purged and maintained with an inert atmosphere of nitrogen, was placed (11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) (400.00 mg, 1.151 mmol, 1.00 equiv), toluene (15.00 mL), methyl 2-bromo-4-(4-[[2-(4-chlorophenyl)-4,4-dimethylcyclohex-1-en-1-yl]methyl]piperazin-1-yl)benzoate (1.22 g, 2.294 mmol, 1.99 equiv), Pd₂(dba)₃.CHCl₃ (237.00 mg, 0.229 mmol, 0.20 equiv), Xantphos (266.00 mg, 0.460 mmol, 0.40 equiv), Cs₂CO₃ (1.12 g, 3.437 mmol, 2.99 equiv). The resulting solution was stirred for 3 hr at 100 degrees C. in an oil bath. The reaction mixture was cooled to room temperature. The resulting mixture was concentrated under vacuum. The residue was applied onto a silica gel column with ethyl acetate/petroleum ether (1:2). This resulted in 820 mg (89.21%) of methyl 4-(4-[[2-(4-chlorophenyl)-4,4-dimethylcyclohex-1-en-1-yl]methyl]piperazin-1-yl)-2-[(11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) as a yellow solid. LC-MS: (ES, m/z) M+1=798.

Synthesis of methyl 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }11,15]]hexadeca-1(9),2,5,7-tetraen-10-yl]benzoate (assumed): Into a 40-mL vial, was placed methyl 4-(4-[[2-(4-chlorophenyl)-4,4-dimethylcyclohex-1-en-1-yl]methyl]piperazin-1-yl)-2-[(11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) (820.00 mg, 1.027 mmol, 1.00 equiv), TBAF in THE (15.00 mL), ethylenediamine (1.30 g, 21.631 mmol, 21.06 equiv). The resulting solution was stirred for 8 hr at 70 degrees C. in an oil bath. The reaction mixture was cooled to room temperature. The resulting mixture was concentrated under vacuum. The residue was applied onto a silica gel column with ethyl acetate/petroleum ether (2:1). This resulted in 380 mg (55.37%) of methyl 4-(4-[[2-(4-chlorophenyl)-4,4-dimethylcyclohex-1-en-1-yl]methyl]piperazin-1-yl)-2-[(11 S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) as a light yellow solid. LC-MS: (ES, m/z) M+1=668.

Synthesis of 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed): Into a 40-mL vial, was placed methyl 4-(4-[[2-(4-chlorophenyl)-4,4-dimethylcyclohex-1-en-1-yl]methyl]piperazin--yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) (150.00 mg, 0.224 mmol, 1.00 equiv), dioxane (3.00 mL), MeOH (3.00 mL), NaOH (0.60 mL, 2.400 mmol, 10.69 equiv). The resulting solution was stirred for 4 hr at 70 degrees C. in an oil bath. The reaction mixture was cooled to room temperature. The resulting mixture was concentrated under vacuum. The pH value of the solution was adjusted to 5-6 with HCl (2 mol/L). The resulting solution was extracted with 3×50 mL of ethyl acetate and the organic layers combined. The resulting mixture was washed with 1×300 mL of brine. The mixture was dried over anhydrous sodium sulfate and concentrated under vacuum. The residue was purified by Prep-TLC with dichloromethane/methanol (10:1). This resulted in 80 mg (54.48%) of 4-(4-[[2-(4-chlorophenyl)-4,4-dimethylcyclohex-1-en-1-yl]methyl]piperazin-1-yl)-2-[(11 S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed) as a white solid. LC-MS: (ES, m/z) M+1=654.

Synthesis of 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }11,15]]hexadeca-1(9),2,5,7-tetraen-10-yl]-N-(4-[[(2S)-1,4-dioxan-2-ylmethyl]amino]-3-nitrobenzenesulfonyl)benzamide (assumed): Into a 40-mL vial, was placed 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed) (50.00 mg, 0.076 mmol, 1.00 equiv), DCM (5.00 mL), 4-[[(2S)-1,4-dioxan-2-ylmethyl]amino]-3-nitrobenzenesulfonamide (24.00 mg, 0.076 mmol, 0.99 equiv), EDCI (29.00 mg, 0.151 mmol, 1.98 equiv), DMAP (37.00 mg, 0.303 mmol, 3.96 equiv). The resulting solution was stirred overnight at 30 degrees C. in an oil bath. The resulting mixture was concentrated under vacuum. The crude product was purified by Prep-HPLC with the following conditions (Prep-HPLC-006): Column, XBridge Shield RP18 OBD Column, 5 um, 19*150 mm; mobile phase, Water (0.05% NH₃.H₂O) and ACN (40% Phase B up to 70% in 7 min); Detector, UV 254/220 nm. This resulted in 25 mg (34.31%) of 4-(4-[[2-(4-chlorophenyl)-4,4-dimethylcyclohex-1-en-1-yl]methyl]piperazin-1-yl)-2-[(11 S,15R)-13,16-dioxa-2,4,10-triazatetracyclo[7.7.0.0\hat{ }[3,7].0\hat{ }[11,15]]hexadeca-1(9),2,5,7-tetraen-10-yl]-N-(4-[[(2S)-1,4-dioxan-2-ylmethyl]amino]-3-nitrobenzenesulfonyl)benzamide (assumed) as a yellow solid. LC-MS: (ES, m/z) M+1=953. ¹H NMR (300 MHz, DMSO-d₆, ppm) δ12.27 (s, 1H), 11.05 (d, J=66.1 Hz, 1H), 8.42 (d, J=30.6 Hz, 1H), 8.32 (d, J=2.3 Hz, 1H), 7.57 (s, 1H), 7.36 (dd, J=8.4, 4.1 Hz, 3H), 7.08 (ddd, J=14.1, 5.9, 2.6 Hz, 3H), 6.78 (t, J=29.3 Hz, 3H), 6.57 (s, 1H), 6.06-5.94 (m, 1H), 4.45 (dd, J=98.7, 9.1 Hz, 3H), 3.96-3.34 (m, 12H), 3.19 (d, J=16.7 Hz, 4H), 2.81-2.71 (m, 2H), 2.24 (d, J=19.4 Hz, 6H), 1.98 (s, 2H), 1.41 (s, 2H), 0.94 (s, 6H).

### Compound 2-17: Preparation of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(12R)-12-methyl-13-oxa-2,4,10-triazatricyclo[7.4.0.0\hat{ }[3,7]]trideca-1(9),2,5,7-tetraen-10-yl]benzamide

Synthesis of (E)-2-(2-ethoxyvinyl)-4,4,5,5-tetramethyl-1,3,2-dioxaborolane: Into a 3L 4-necked round-bottom flask, was placed ethyl vinyl ether (2000.00 g, 27777.778 mmol, 3.00 equiv), 4,4,5,5-tetramethyl-1,3,2-dioxaborolane (508.00 g, 9259.259 mmol, 1.00 equiv). This solution was cooled to 5 degrees C. in an ice/salt bath. This followed by the addition of Pd(OAc)₂ (10.39 g, 46.296 mmol, 0.005 equiv) in portions at 0-5 degrees C. The resulting solution was stirred for 18h at room temperature. The resulting mixture was concentrated. This resulted in 1500 g (crude) and purify by rectification, collect 65-70 degree at 25 mm Hg, got 890 g(Y=50%, LCMS OK, Q-NMR=87%) of 2-[(Z,E mixtures)-2-ethoxyethenyl]-4,4,5,5-tetramethyl-1,3-dioxolane as light yellow oil. LC-MS: (ES, m/z): M+1=199, R.T=1.979 min, 2.440 min. H-NMR: (300 MHz, DMSO-d₆, ppm): δ6.71-6.95 (m, 1H), 4.29-4.34 (m, 1H), 3.80-3.93 (m, 2H), 1.15-1.21 (m, 16H).

Synthesis of N-[(2R)-2-hydroxypropyl]acetamide: Into a 250-mL 3-necked round-bottom flask purged and maintained with an inert atmosphere of nitrogen, was placed (2R)-1-aminopropan-2-ol (10 g, 133.136 mmol, 1 equiv), DCM (100 mL), TEA (16 g, 159.764 mmol, 1.2 equiv). This was followed by the addition of a solution of acetyl acetate (13.6 g, 133.136 mmol, 1.0 equiv) in DCM (10 mL) dropwise with stirring at 0° C. The resulting solution was stirred for 14 hr at room temperature. The resulting mixture was concentrated. The residue was applied onto a silica gel column with dichloromethane/methanol (100:5). This resulted in 13.5 g (69.25%) of N-[(2R)-2-hydroxypropyl]acetamide as a yellow oil. LC-MS: (ES, m/z): M+1=118.

Synthesis of 4-([[(2S)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as a yellow solid. LC-MS: (ES, m/z): M+1=318, R,T=0.741 min.

Synthesis of methyl 2-bromo-4-(4-((2-(4-chlorophenyl)-4,4-dimethylcyclohex-1-enyl)methyl)piperazin-1-yl)benzoate: Into a 20000-mL round-bottom flask, was placed 1-((2-(4-chlorophenyl)-4,4-dimethylcyclohex-1-enyl)methyl)piperazine dihydrochloride (600 g, 1.53 mol, 1 equiv), methyl 2-bromo-4-fluorobenzoate (357 g, 1.53 mol, 1 equiv), DBU (319 g, 6.12 mol, 4 equiv) and DMSO (8000 mL). The resulting solution was stirred for 20 h at 70 degrees C. LCMS showed material was completely consumed. The resulting mixture was cooled to R,T and poured into water (32 L). The mixture was filtrated, collection of filter cake and the filter cake was washed by water (3000 mL×3) and dried by oven to give product 740 g (Y=91%) methyl 2-bromo-4-(4-((2-(4-chlorophenyl)-4,4-dimethylcyclohex-1-enyl)methyl)piperazin-1-yl)benzoate as a white solid. H-NMR: (300 MHz, DMSO-d₆,ppm) δ: 7.73 (d, J=9.0 Hz, 1H), 7.42-7.39 (m, 2H), 7.18-7.12 (m, 3H), 6.97-6.94 (m, 1H), 4.00-3.84 (m, 2H), 3.76 (s, 2H), 3.57 (s, 3H), 3.51-3.33 (m, 4H), 2.79-2.60 (m, 2H), 2.32-2.30 (m, 2H), 2.03-1.97 (m, 2H), 1.47-1.45 (m, 2H), 0.96 (s, 6H).

Synthesis of 2-bromo-4-(4-((2-(4-chlorophenyl)-4,4-dimethylcyclohex-1-enyl)methyl)piperazin-1-yl)benzoic acid: Into a 20000-mL round-bottom flask, was placed methyl 2-bromo-4-(4-((2-(4-chlorophenyl)-4,4-dimethylcyclohex-1-enyl)methyl)piperazin-1-yl)benzoate (730 g, 1.37 mol, 1 equiv), LiOH (131.5 g, 5.48 mol, 4 equiv) and MeOH/THF/water (4500 mL/3000 mL/1000 mL). The resulting solution was stirred for 16 h at 70 degrees C. LCMS showed material was completely consumed. The resulting mixture was cooled to R,T and concentrated. The residue was diluted with water (5000 mL) and the mixture was adjust PH to 3-5 with HCl (6 M), followed by filtrated, collection of filter cake and dried by oven to give product 650 g 2-bromo-4-(4-((2-(4-chlorophenyl)-4,4-dimethylcyclohex-1-enyl)methyl)piperazin-1-yl)benzoic acid as a white solid. H-NMR-PH-PHNW-4-55-400: (300 MHz, DMSO-d₆,ppm) δ: 10.60 (bs, 1H), 7.73 (d, J=8.4 Hz, 1H), 7.42-7.39 (m, 2H), 7.14-7.11 (m, 3H), 6.95-6.92 (m, 1H), 4.00-3.84 (m, 2H), 3.76 (s, 2H), 3.51-3.33 (m, 4H), 2.79-2.60 (m, 2H), 2.32-2.30 (m, 2H), 2.03-1.97 (m, 2H), 1.47-1.45 (m, 2H), 0.97 (s, 6H).

Synthesis of 5-bromo-6-fluoropyridin-2-amine: Into a 1OOL 4-necked round-bottom flask, was placed 6-fluoropyridin-2-amine (4500.00 g, 40178.571 mmol, 1.00 equiv), ACN (25000.00 mL). This was followed by the addition of NBS (7100.00 g, 41764.706 mmol, 1.03 equiv) in portions (2 h) at 5-15 degrees C. The resulting solution was stirred for 3h at room temperature. The resulting solution was diluted with 50 L of water. The resulting solution was extracted with 2×32 L of ethyl acetate. The resulting mixture was washed with 10 L of brine. The mixture was dried over anhydrous sodium sulfate and concentrated. The resulting mixture was washed with 3×13L of PE. This mixture was dried by oven to give product 6900 g(Y=90%) of 5-bromo-6-fluoropyridin-2-amine as a light brown solid. LC-MS: (ES, m/z): M+1=191, 193, R.T=0.851 min. H-NMR: (300 MHz, DMSO-d₆, ppm): δ7.63-7.71 (m, 1H), 6.57 (s, 2H), 6.27-631 (m, 1H).

Synthesis of 5-bromo-6-fluoro-3-iodopyridin-2-amine: Into a 100 L 4-necked round-bottom flask, was placed 5-bromo-6-fluoropyridin-2-amine (6500.00 g, 34219.531 mmol, 1.00 equiv), AcOH (39000.00 mL). The solution was cooled to 15 degree C. in a water/ice bath. This was followed by the addition of NIS (8470.00 g, 37641.484 mmol, 1.10 equiv) in portions (3h) at 10-20 degrees C. The resulting solution was stirred for 3h at room temperature. The resulting solution was added into 100 L of water. The mixture was filtrated, collection of filter cake and the filter cake was washed by water (25L x 3) and dried by oven to give product 9840 g(Y=85%) of 5-bromo-6-fluoro-3-iodopyridin-2-amine as a brown solid. LC-MS: (ES, m/z): M+1=317, 319, R.T=1.072 min. H-NMR: (300 MHz, DMSO-d₆, ppm): δ8.17-8.20 (m, 1H), 6.69 (s, 2H).

Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO4 (1720.00 g, 8113.207 mmol, 3.00 equiv), Ruphos (12.00 g, 27.060 mmol, 0.02 equiv), Pd(OAc)₂ (10.00 g, 44.643 mmol, 0.02 equiv). The resulting solution was stirred for 12h at room temperature in a liquid nitrogen bath. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(Z,E mixtures)-2-ethoxyethenyl]-6-fluoropyridin-2-amine as dark brown oil. LC-MS: (ES, m/z): M+1=261, 263, R.T=1.090 min.

Synthesis of 5-bromo-6-fluoro-1H-pyrrolo [2, 3-b]pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 410 g(Y=75%) of 5-bromo-6-fluoro-1H-pyrrolo [2, 3-b]pyridine as a light brown solid.LC-MS: (ES, m/z): M+1=215, 217, R.T=0.993 min. H-NMR: (300 MHz, DMSO-d₆, ppm): δ9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 5L 4-necked round-bottom flask was placed 5-bromo-6-fluoro-1H-pyrrolo [2, 3-b]pyridine (200.00 g, 930.232 mmol, 1.00 equiv), DMF (2500.00 mL). This solution was cooled to 0 degrees C. in a water/ice bath. This was followed by the addition of NaH (75.00 g, 1860.464 mmol, 2.00 equiv) in portions at 0 degrees C. To this was added SEM-Cl (233.00 g, 1395.210 mmol, 1.50 equiv) dropwise with stirring at 0 degrees C. The resulting solution was stirred for 1 h at room temperature. The reaction was then quenched by the addition of 1000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 298 g (87%) of 5-bromo-6-fluoro-1-[[2-(trimethylsilyl) ethoxy]methyl]pyrrolo [2, 3-b]pyridine as light yellow oil. LC-MS: (ES, m/z): M+1=345, 347, R.T=1.435 min. H-NMR (300 MHz, DMSO-d₆, ppm): 6 8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

Synthesis of (R)-N-(2-(5-bromo-1-((2-(trimethylsilyl)ethoxy)methyl)-1H-pyrrolo[2,3-b]pyidin-6-yloxy)propyl)acetamide: Into a 50-mL round-bottom flask, was placed N-[(2R)-2-hydroxypropyl]acetamide (680 mg, 5.8 mmol, 2.00 equiv), dioxane (10 mL). This was followed by the addition of NaH (348 mg, 8.7 mmol, 3 equiv), in portions at 15° C. The resulting solution was stirred for 10 min at RT. To this was added a solution of 5-bromo-6-fluoro-1-((2-(trimethylsilyl)ethoxy)methyl)-1H-pyrrolo[2,3-b]pyridine (1 g, 2.9 mmol, 1 equiv) in dioxane (5 mL). The resulting solution was stirred for 4 hr at 80° C. in an oil bath. The reaction was cooled to r.t then quenched by the addition of 5 mL of water. The resulting solution was extracted with 3×10 mL of ethyl acetate. The resulting mixture was washed with 3×5 ml of H₂O. The resulting mixture was washed with 1×10 mL of NaCl (aq). The mixture was dried over anhydrous sodium sulfate and concentrated. The residue was applied onto a silica gel column with PE/EA (100:20). This resulted in 880 mg (73%) of (R)-N-(2-(5-bromo-1-((2-(trimethylsilyl)ethoxy)methyl)-1H-pyrrolo[2,3-b]pyridin-6-yloxy)propyl)acetamide as a white solid.

Synthesis of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one: Into a 25-mL round-bottom flask purged and maintained with an inert atmosphere of nitrogen, was placed (R)-N-(2-(5-bromo-1-((2-(trimethylsilyl)ethoxy)methyl)-1H-pyrrolo[2,3-b]pyridin-6-yloxy)propyl)acetamide (500 mg, 1.13 mmol, 1 equiv), dioxane (8 mL), Cs₂CO₃ (1.1 g, 3.4 mmol, 3 equiv), BrettPhos Pd G3 (102 mg, 0.11 mmol, 0.10 equiv). The resulting solution was stirred for 14 hr at 80° C. in an oil bath. The resulting mixture was concentrated. The residue was applied onto a silica gel column with ethyl acetate/petroleum ether (50:50). This resulted in 300 mg (73.3%) of 1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one as light yellow oil.

Synthesis of (12R)-4-(trimethylsilyl)ethoxy)methyl)-12-methyl-13-oxa-2,4,10-triazatricyclo[7.4.0.0\hat{ }[3,7]]trideca-1(9),2,5,7-tetraene: Into a 8-mL vial, was placed1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one (50 mg), methanol (0.5 mL), NaOH/H₂O (1M, 0.5 ml). The resulting solution was stirred for 14 hr at 80° C. in an oil bath. The resulting mixture was concentrated. The residue was applied onto a silica gel column with ethyl acetate/petroleum ether (1:1). This resulted in 15 mg (34%) of (12R)-4-(trimethylsilyl)ethoxy) methyl)-12-methyl-13-oxa-2,4,10-triazatricyclo[7.4.0.0\hat{ }[3,7]]trideca-1(9),2,5,7-tetraene as brown oil.

Synthesis of 4-(4-[[2-(4-chlorophenyl)-4,4-dimethylcyclohex-1-en-1-yl]methyl]piperazin-1-yl)-N- (4-[[(2S)-1,4-dioxan-2-ylmethyl]amino]-3-nitrobenzenesulfonyl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide: Into a 8-mL vial purged and maintained with an inert atmosphere of nitrogen, was placed (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (100.00 mg, 0.313 mmol, 1.00 equiv), 2-bromo-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide (255.79 mg, 0.313 mmol, 1.00 equiv), DMF (2.00 mL), CuI (23.84 mg, 0.125 mmol, 0.40 equiv), N₁,N₂-bis(4-hydroxy-2,6-dimethylphenyl)oxalamide (20.53 mg, 0.063 mmol, 0.20 equiv), K₂CO₃ (129.78 mg, 0.939 mmol, 3.00 equiv). The resulting solution was stirred for 4 hr at 100 degrees C. in an oil bath. The resulting solution was diluted with 10 mL of H₂O. The resulting solution was extracted with 3×5 mL of ethyl acetate. The resulting mixture was washed with 3×5 ml of H₂O. The mixture was dried over anhydrous sodium sulfate. The residue was applied onto a silica gel column and eluted with dichloromethane/methanol (10:1). This resulted in 220 mg (66.57%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide as a yellow solid. LC-MS: (ES, m/z): 1055.5 [M+H].

Synthesis of 4-(4-[[2-(4-chlorophenyl)-4,4-dimethylcyclohex-1-en-1-yl]methyl]piperazin-1-yl) -N-(4-[[(2S)-1,4-dioxan-2-ylmethyl]amino]-3-nitrobenzenesulfonyl)-2-[(12R)-12-methyl-13-oxa-2,4,10-triazatricyclo[7.4.0.0\hat{ }[3,7]]trideca-1(9),2,5,7-tetraen-10-yl]benzamide: Into a 25-mL round-bottom flask, was placed 4-(4-[[2-(4-chlorophenyl)-4,4-dimethylcyclohex-1-en-1-yl]methyl]piperazin-1-yl)-N- (4-[[(2S)-1,4-dioxan-2-ylmethyl]amino]-3-nitrobenzenesulfonyl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide (210.00 mg), ethane-1,2-diamine (1.00 mL), TBAF in THE (3M, 5.00 mL). The resulting solution was stirred for 14 hr at 70 degrees C. in an oil bath. The resulting mixture was concentrated. The resulting solution was diluted with 20 mL of DCM. The resulting mixture was washed with 3×5 ml of H₂O. The mixture was dried over anhydrous sodium sulfate. The residue was purified with Prep-TLC with dichloromethane/methanol (100:5). This resulted in 70 mg (38.02%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(12R)-12-methyl-13-oxa-2,4,10-triazatricyclo[7.4.0.0\hat{ }[3,7]]trideca-1(9),2,5,7-tetraen-10-yl]benzamide as a yellow solid. LC-MS: (ES, m/z): 925.4 [M+H]. ¹H NMR (300 MHz, CDCl₃, ppm): δ12.38 (s, 1H), 8.68-8.46 (m, 3H), 8.16-7.97 (m, 1H), 7.90-7.81 (m, 1H), 7.28-7.05 (m, 3H), 7.02-6.78 (m, 3H), 6.77-6.63 (m, 2H), 6.54 (s, 1H), 6.11-6.03 (m, 1H), 4.98-4.83 (m, 1H), 4.10-2.99 (m, 17H), 2.91-2.79 (m, 2H), 2.40-2.17 (m, 6H), 2.10-2.02 (m, 2H), 1.70-1.61 (m, 3H), 0.97 (s, 6H).

### Compound 2-18: Preparation of 4-(4-[[2-(4-chlorophenyl)-4,4-dimethylcyclohex-1-en-1-yl]methyl]piperaz in-1-yl)-N-(4-[[(2S)-1,4-dioxan-2-ylmethyl]amino]-3-nitrobenzenesulfonyl)-2-[11-oxo-13-oxa-2,4,10-triazatricyclo[7.4.0. 0\hat{ }[3,7]]trideca-1(9),2,5,7-tetraen-10-yl]benzamide

Synthesis of 5-bromo-6-fluoropyridin-2-amine. Into a 5000-mL 3-necked round-bottom flask, was placed 6-fluoropyridin-2-amine (220 g, 2 mol, 1.00 equiv), CH₃CN (2.0 L), NBS (420 g, 2.2 mol, 1.20 equiv). The resulting solution was stirred for overnight at room temperature. The reaction was then quenched by the addition of 2 L of water. The resulting solution was extracted with 3×1 L of ethyl acetate and the organic layers combined. The resulting organic phase was washed with 3×1 L of brine. The mixture was dried over anhydrous sodium sulfate, then filtered and concentrated under vacuum. The residue was applied onto a silica gel column with ethyl acetate/petroleum ether (0:1-1:5). This resulted in 200 g (60%) of 5-bromo-6-fluoropyridin-2-amine as a white solid. LCMS: (ES, m/z): M+1: 191, 193.

Synthesis of 5-bromo-6-fluoro-3-iodopyridin-2-amine. Into a 2000-mL 4-necked round-bottom flask, was placed 5-bromo-6-fluoropyridin-2-amine (200 g, 1.05 mol, 1.00 equiv) in AcOH (1500 mL), iodo(sulfanyl)amine(NIS) (200 g, 1.15 mol, 1.10 equiv). The resulting solution was stirred for overnight at room temperature. The reaction was then quenched by the addition of 3000 mL of water. The solids were collected by filtration and wash by Et₂O. This resulted in 170 g (50%) of 5-bromo-6-fluoro-3-iodopyridin-2-amine as a white solid. The organic phase was concentrated under vacuum, result in 150 g crude product oil. ¹H-NMR: (CDCl₃, 300 MHz) δ: 7.98 (d, J=14.4 Hz, 1H), 4.94-5.00 (bs, 2H).

Synthesis of 5-bromo-6-fluoro-3-[2-(trimethylsilyl)ethynyl]pyridin-2-amine. Into a 3000-mL 3-necked round-bottom flask, was placed a solution of 5-bromo-6-fluoro-3-iodopyridin-2-amine (170 g, 536.45 mmol, 1.00 equiv) in tetrahydrofuran (1500 mL), CuI (10.2 g, 53.56 mmol, 0.10 equiv), TEA (500 mL), dichloropalladium; bis(triphenylphosphane) (11.2 g, 15.96 mmol, 0.03 equiv), ethyl(ethynyl)dimethylsilane (63 g, 561.27 mmol, 1.20 equiv). The resulting solution was stirred for 16 hours at room temperature. The reaction was then quenched by the addition of 2000 mL of water. The resulting solution was extracted with 3×1000 mL of ethyl acetate and the organic layers combined. The resulting mixture was washed with 3×1000 mL of brine. The mixture was dried over anhydrous sodium sulfate, then filtered and concentrated under vacuum. The residue was applied onto a silica gel column with ethyl acetate/petroleum ether (0:1-1:1). This resulted in 120 g (78%) of 5-bromo-6-fluoro-3-[2-(trimethylsilyl)ethynyl]pyridin-2-amine as yellow oil. LC-MS (ES, m/z): M+1: 289, 287.

Synthesis of N-[5-bromo-6-fluoro-3-[2-(trimethylsilyl)ethynyl]pyridin-2-yl]acetamide. Into a 2000-mL 4-necked round-bottom flask, was placed a solution of 5-bromo-6-fluoro-3-[2-(trimethylsilyl)ethynyl]pyridin-2-amine (84 g, 292.48 mmol, 1.00 equiv) in dichloromethane (1000 mL), pyridine (57.8 g, 730.72 mmol, 2.50 equiv). This was followed by the addition of acetyl chloride (50.2 g, 639.51 mmol, 2.20 equiv) dropwise with stirring at 0 degree. The resulting solution was stirred for overnight at room temperature. The reaction was then quenched by the addition of 1000 mL of water. The resulting mixture was washed with 2×1000 mL of water. The mixture was dried over anhydrous sodium sulfate and concentrated under vacuum. The residue was applied onto a silica gel column with ethyl acetate/petroleum ether (0:1-1:10). This resulted in 80 g (83%) of N-[5-bromo-6-fluoro-3-[2-(trimethyl silyl)ethynyl]pyridin-2-yl]acetamide as a white solid. LC-MS: (ES, m/z): M+1=331.

Synthesis of 5-bromo-6-fluoro-1H-pyrrolo[2,3-b]pyridine. Into a 2000-mL round-bottom flask, was placed a solution of N-[5-bromo-6-fluoro-3-[2-(trimethylsilyl)ethynyl]pyridin-2-yl]acetamide (80 g, 242.98 mmol, 1.00 equiv) in tetrahydrofuran (300 mL), TBAF(1M in tetrahydrofuran) (729 mL, 3.00 equiv). The resulting solution was stirred for 12 h at 70 degree. The reaction mixture was cooled to room temperature. The resulting mixture was concentrated under vacuum. The reaction was then quenched by the addition of 500 mL of water. The resulting solution was extracted with 3×300 mL of ethyl acetate and the organic layers combined. The resulting mixture was washed with 3×300 mL of brine. The mixture was dried over anhydrous sodium sulfate and concentrated under vacuum. The residue was applied onto a silica gel column with ethyl acetate/petroleum ether (0:1-4:1). This resulted in 15 g (29%) of 5-bromo-6-fluoro-1H-pyrrolo[2,3-b]pyridine as a white solid. LC-MS: (ES, m/z): M+1=213.

Synthesis of 5-bromo-6-fluoro-1-[[2-(trimethylsilyl)ethoxy]methyl]-1H-pyrrolo[2,3-b]pyridine. Into a 250-mL 3-necked round-bottom flask purged and maintained with an inert atmosphere of nitrogen, was placed a solution of 5-bromo-6-fluoro-1H-pyrrolo[2,3-b]pyridine (15 g, 69.76 mmol, 1.00 equiv) in N,N-dimethylformamide (150 mL). This was followed by the addition of sodium hydride (4.2 g, 175.00 mmol, 1.50 equiv), in portions at 0 degree. After 0.5 h stirring, to this was added SEM-Cl (14 g, 84.34 mmol, 1.20 equiv) dropwise with stirring at 0 degree. The resulting solution was allowed to react, with stirring, for an additional 3 h at room temperature. The reaction was then quenched by the addition of 300 mL of water. The resulting solution was extracted with 3×200 mL of ethyl acetate and the organic layers combined. The resulting mixture was washed with 3×200 mL of brine. The mixture was dried over anhydrous sodium sulfate and concentrated under vacuum. The residue was applied onto a silica gel column with ethyl acetate/petroleum ether (0:1-1:10). This resulted in 15 g (62%) of 5-bromo-6-fluoro-1-[[2-(trimethylsilyl)ethoxy]methyl]-1H-pyrrolo[2,3-b]pyridine as yellow oil.

Synthesis of 6-(tert-butoxy)-N-(diphenylmethylidene)-1-[[2-(trimethylsilyl)ethoxy]methyl]-1H-pyrrolo[2,3-b]pyridin-5-amine. Into a 250-mL round-bottom flask, was placed a solution of 5-bromo-6-fluoro-1-[[2-(trimethylsilyl)ethoxy]methyl]-1H-pyrrolo[2,3-b]pyridine (20.7 g, 60 mmol, 1.00 equiv) in dioxane (300 mL), t-BuOK (20.5 g, 180 mmol, 3.00 equiv), xantphos (6.9 g, 12 mmol, 0.20 equiv), Pd₂(dba)₃.CHCl₃ (5.7 g, 0.10 equiv), diphenylmethanimine (14.04 g, 78 mmol, 1.20 equiv). The resulting solution was stirred for overnight at 100° C. The resulting mixture was concentrated under vacuum. The residue was applied onto a silica gel column with ethyl acetate/petroleum ether (0:1-1:20). This resulted in 15 g (crude) of 6-(tert-butoxy)-N-(diphenylmethylidene)-1-[[2-(trimethylsilyl)ethoxy]methyl]-1H-pyrrolo[2,3-b]pyridin-5-amine as white oil. LC-MS: (ES, m/z): M+1=500.

Synthesis of 5-amino-1-[[2-(trimethylsilyl)ethoxy]methyl]-1H-pyrrolo[2,3-b]pyridin-6-ol hydrogen chloride salt. Into a 500-mL round-bottom flask, was placed 6-(tert-butoxy)-N-(diphenylmethylidene)-1-[[2-(trimethylsilyl)ethoxy]methyl]-1H-pyrrolo[2,3-b]pyridin-5-amine (15 g, 30.02 mmol, 1.00 equiv) in dioxane (120 mL), HCl/dioxane(4M, 30 mL). The resulting solution was stirred for 5 h at room temperature. The resulting solution was diluted with 500 mL of ether. The solids were collected by filtration. This resulted in 5 g (crude) of 5-amino-1-[[2-(trimethylsilyl)ethoxy]methyl]-1H-pyrrolo[2,3-b]pyridin-6-ol hydrogen chloride salt as a red solid, Q-NMR show it might converted into 3 hydrogen chloride salt. LC-MS: (ES, m/z): M+1=280.

Synthesis of 4-[[2-(trimethylsilyl)ethoxy]methyl]-13-oxa-2,4,10-triazatricyclo[7.4.0.0\hat{ }[3,7]]trideca-1,3(7),5,8-tetraen-11-one. Into a 250-mL round-bottom flask, was placed 5-amino-1-[[2-(trimethylsilyl) ethoxy]methyl]-1H-pyrrolo[2,3-b]pyridin-6-ol hydrogen chloride salt (5 g, 17.89 mmol, 1.00 equiv) in N,N-dimethylformamide (100 mL), potassium carbonate (7.4 g, 53.54 mmol, 3.00 equiv), and add 2-chloroacetyl chloride (4 g, 35.42 mmol, 2.00 equiv) at 0 degree by dropwise. The resulting solution was stirred for overnight at 70° C. The reaction mixture was cooled to room temperature. The reaction was then quenched by the addition of 200 mL of water. The resulting solution was extracted with 3×100 mL of ethyl acetate and the organic layers combined. The resulting mixture was washed with 3×100 mL of brine. The mixture was dried over anhydrous sodium sulfate and concentrated under vacuum. The residue was applied onto a silica gel column with ethyl acetate/petroleum ether (0:1-1:1). This resulted in 2.5 g (44%) of 4-[[2-(trimethylsilyl)ethoxy]methyl]-13-oxa-2,4,10-triazatricyclo[7.4.0.0\hat{ }[3,7]]trideca-1,3(7),5,8-tetraen-11-one as a white solid. LC-MS: (ES, m/z): M+1=320.

Synthesis of methyl 2-bromo-4-(4-((2-(4-chlorophenyl)-4,4-dimethylcyclohex-1-enyl)meth yl)piperazin-1-yl)benzoate. Into a 2000-mL round-bottom flask, was placed 1-((2-(4-chlorophenyl)-4,4-dimethylcyclohex-1-enyl)methyl)piperazine dihydrochloride (60 g, 0.153 mol, 1 equiv), methyl 2-bromo-4-fluorobenzoate (35.7 g, 0.153 mol, 1 equiv), DBU (31.9 g, 0.612 mol, 4 equiv) and DMSO (800 mL). The resulting solution was stirred for 20 h at 70 degrees C. LCMS showed material was completely consumed. The resulting mixture was cooled to R.T and poured into water (3 L). The mixture was filtrated, collection of filter cake and the filter cake was washed by water (300 mL×3) and dried by oven to give product 74 g (Y: 91%) methyl 2-bromo-4-(4-((2-(4-chlorophenyl)-4,4-dimethylcyclohex-1-enyl)methyl)piperazin-1-yl)benzoate as a white solid.

Synthesis of 2-bromo-4-(4-((2-(4-chlorophenyl)-4,4-dimethylcyclohex-1-enyl)methyl)piperazin-1-yl)benzoic acid. Into a 2000-mL round-bottom flask, was placed methyl 2-bromo-4-(4-((2-(4-chlorophenyl)-4,4-dimethylcyclohex-1-enyl)methyl)piperazin-1-yl)benzoate (73 g, 0.137 mol, 1 equiv), LiOH (13.15 g, 0.548 mol, 4 equiv) and MeOH/THF/water (450 mL/300 mL/100 mL). The resulting solution was stirred for 16 h at 70 degrees C. LCMS showed material was completely consumed. The resulting mixture was cooled to R.T and concentrated. The residue was diluted with water (500 mL) and the mixture was adjust PH to 3-5 with HCl (6 M), followed by filtrated, collection of filter cake and dried by oven to give product 65 g (Y: 93%)₂-bromo-4-(4-((2-(4-chlorophenyl)-4,4-dimethylcyclohex-1-enyl)methyl)piperazin -1-yl)benzoic acid as a white solid.

Synthesis of 2-bromo-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide. Into a 2000-mL round-bottom flask, was placed 2-bromo-4-(4-((2-(4-chlorophenyl)-4,4-dimethylcyclohex-1-enyl)methyl) piperazin-1-yl)benzoic acid (55 g, 0.107 mol, 1 equiv), DCM (1 L), (S)-4-(((1,4-dioxan-2-yl)methyl)amino)-3-nitrobenzenesulfonamide (32 g, 0.102 mol, 0.95 equiv), EDCI (30.8 g, 0.161 mol, 1.5 equiv), DMAP (52.2 g, 0.428 mol, 4 equiv). The resulting solution was stirred for overnight at 25 degrees C. LCMS showed material was completely consumed. The resulting mixture is followed by dilute hydrochloric acid (1.0 M) (100 mL×3), saturated sodium bicarbonate (100 mL×3) and brine (100 mL×1), and then the organic phase was dried by Na₂SO₄, filtrated. The filtrate was concentrated to give product 81 g (Y: 93%) as a light brown yellow solid 2-bromo-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide as a brown yellow solid. LC-MS: (ES, m/z): M+1=816/819, R.T=2.01 min.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(11-oxo-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl)benzamide. Into a 40-mL round-bottom flask, was placed 2-bromo-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide (214.89 mg, 0.263 mmol, 1.2 equiv), 4-[[2-(trimethylsilyl)ethoxy]methyl]-13-oxa-2,4,10-triazatricyclo[7.4.0.0\hat{ }[3,7]]trideca-1(9),2,5,7-tetraen-11-one (70.00 mg, 0.219 mmol, 1.00 equiv), 4,7-dimethoxy-1,10-phenanthroline (26.33 mg, 0.110 mmol, 0.5 equiv), Cs₂CO₃ (214.20 mg, 0.657 mmol, 3 equiv), Dioxane (10.00 mL), CuI (20.87 mg, 0.110 mmol, 0.5 equiv). The resulting solution was stirred for 3 h at 110 degrees C. The resulting mixture was concentrated. The residue was applied onto a silica gel column with ethyl acetate/petroleum ether (0:1-1:1). This resulted in 200 mg (86.45%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(11-oxo-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl)benzamide as a yellow crude solid. LC-MS: (ES, m/z): M+1=1055.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[11-oxo-13-oxa-2,4,10-triazatricyclo [7.4.0.0\hat{ }[3,7]]trideca-1(9),2,5,7-tetraen-10-yl]benzamide. Into a 40-mL round-bottom flask, was placed 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(11-oxo-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl)benzamide (200.00 mg, 0.189 mmol, 1.00 equiv), THE (10 mL), ethylenediamine (227.71 mg, 3.789 mmol, 20.00 equiv), TBAF (990.65 mg, 3.789 mmol, 20 equiv). The resulting solution was stirred for 12 h at 70 degrees C. The reaction was then quenched by the addition of 10 mL of water. The resulting solution was extracted with 2×10 mL of ethyl acetate concentrated. The crude product was purified by Prep-HPLC with the following conditions (Waters-2767): Column, X-bridge RP18, 5 um, 19*100 mm; mobile phase, 0.03% acerbity in water (0.03% HCl) and CH₃CN (32% CH₃CN up to 52% in 6 min); Detector, UV 254 nm. This resulted in 25 mg (14.26%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[11-oxo-13-oxa-2,4,10-triazatricyclo[7.4.0.0\hat{ }[3,7]]trideca-1(9),2,5,7-tetraen-10-yl]benzamide as a yellow solid. LC-MS: (ES, m/z): M+1=925. ¹H NMR (300 MHz, DMSO-d₆) δ 11.32 (d, J=7.2 Hz, 1H), 8.52 (s, 1H), 8.37 (dd, J=4.6, 2.3 Hz, 1H), 7.72 (d, J=8.5 Hz, 1H), 7.65 (s, 1H), 7.41 (d, J=8.1 Hz, 2H), 7.19 (d, J=5.8 Hz, 1H), 7.11 (d, J=8.3 Hz, 2H), 6.92 (d, J=9.7 Hz, 2H), 6.60 (d, J=1.8 Hz, 1H), 6.15-6.07 (m, 1H), 4.67 (d, J=15.0 Hz, 1H), 4.36 (s, 1H), 3.98-3.75 (m, 5H), 3.75-3.47 (m, 7H), 3.32 (s, 4H), 2.80 (s, 2H), 2.05 (s, 2H), 1.48 (s, 2H), 0.96 (s, 6H).

### Compound 3-1: Preparation of 4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide

Synthesis of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 100-mL round-bottom flask purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (999.0 mg, 1.88 mmol, 4.00 equiv), toluene (20 mL), 4-[[2-(trimethylsilyl)ethoxy]methyl]-14-oxa-2,4,10-triazatricyclo[7.5.0.0\hat{ }[3,7]]tetradeca-1(9),2,5,7-tetraene (150 mg, 0.47 mmol, 1 equiv), Cs₂CO₃ (764.9 mg, 2.35 mmol, 5 equiv), XantPhos Pd 2G (333.2 mg, 0.38 mmol, 0.8 equiv). The resulting solution was stirred for 1 overnight at 110° C. The resulting solution was diluted with 300 mL of water. The resulting solution was extracted with 2×100 mL of ethyl acetate. The resulting mixture was washed with 1×300 mL of brine. The mixture was dried over anhydrous sodium sulfate and concentrated under vacuum. The residue was applied onto a silica gel column with ethyl acetate/petroleum ether (1:2). This resulted in 200 mg (55.29%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as a yellow solid. LC-MS: (ES, m/z): M+H=769, R,T=3.076 min. The measurements of the retention were done with a reversed phase column (C18). Shimadzu LCMS 2020; 50*3.0 Shim-pack XR-ODS, 2.2 microm; Eluent A: water (0.05% TFA); Eluent B: Acetonitrile; linear gradient from 5% acetonitrile to 100% acetonitrile in 5.0 minutes; Oven temperature 40° C.; flow: 1.5 mL/min.

Synthesis of methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate: Into a 40-mL vial, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-1 (200 mg, 0.26 mmol, 1 equiv), THE (20 mL), TBAF.3H₂O (2.5 g), ethane-1,2-diamine (1.5 g, 24.96 mmol, 96.15 equiv). The resulting solution was stirred for 2 overnight at 70° C. in an oil bath. The resulting solution was diluted with 200 mL of water. The resulting solution was extracted with 3×30 mL of ethyl acetate. The resulting mixture was washed with 2×200 mL of brine. The mixture was dried over anhydrous sodium sulfate and concentrated under vacuum. The residue was applied onto a silica gel column with ethyl acetate/petroleum ether (2:1). This resulted in 130 mg (78.22%) of methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate as a light yellow solid. LC-MS: (ES, m/z): M+H=639, R,T=1.388 min. The measurements of the retention were done with a reversed phase column (C18). Shimadzu LCMS 2020; 50*3.0 Shim-pack XR-ODS, 2.2 microm; Eluent A: water (0.05% TFA); Eluent B: Acetonitrile; linear gradient from 5% acetonitrile to 100% acetonitrile in 2.6 minutes; Oven temperature 40° C.; flow: 1.5 mL/min.

Synthesis of 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid: Into a 40-mL vial, was placed methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate (130 mg, 0.20 mmol, 1 equiv), MeOH (6 mL), THE (6 mL), H₂O (2 mL), NaOH (81.2 mg, 2.03 mmol, 10.00 equiv). The resulting solution was stirred for 1 overnight at 60° C. in an oil bath. The resulting mixture was concentrated under vacuum. The pH value of the solution was adjusted to 5-6 with HCl (2 mol/L). The resulting solution was extracted with 2×50 mL of dichloromethane/MeOH (v:v=10:1). The resulting mixture was washed with 2×200 mL of brine. The mixture was dried over anhydrous sodium sulfate and concentrated under vacuum. This resulted in 80 mg (62.92%) of 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid as a light yellow solid. LC-MS: (ES, m/z): M+H=625, R,T=1.336 min. The measurements of the retention were done with a reversed phase column (C18). Shimadzu LCMS 2020; 50*3.0 Shim-pack XR-ODS, 2.2 microm; Eluent A: water (0.05% TFA); Eluent B: Acetonitrile; linear gradient from 5% acetonitrile to 100% acetonitrile in 2.6 minutes; Oven temperature 40° C.; flow: 1.5 mL/min.

Synthesis of 4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)-2-[14-oxa-2,4,10-triazatricyclo[7.5.0.0\hat{ }[3,7]]tetradeca-1(9),2,5,7-tetraen-10-yl]benzamide: Into a 40-mL round-bottom flask, was placed 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid (50 mg, 0.08 mmol, 1 equiv), DCM (3 mL), 3-nitro-4-[[(oxan-4-yl)methyl]amino]benzene-1-sulfonamide (25.2 mg, 0.08 mmol, 1.00 equiv), EDCI (30.6 mg, 0.16 mmol, 2 equiv), DMAP (39.0 mg, 0.32 mmol, 4 equiv). The resulting solution was stirred for overnight at 25 degrees C. The resulting mixture was concentrated under vacuum. The residue was applied onto a silica gel column with dichloromethane/methanol (10:1). The crude product was purified by Flash-Prep-HPLC with the following conditions (IntelFlash-1): Column, C18 reversed phase column; mobile phase, Water (10 MMOL/L NH₄HCO₃+0.05% NH₃.H₂O) and CH₃CN (20.0% CH₃CN up to 90.0% in 30 min); Detector, UV 220 nm. This resulted in 19.1 mg (25.90%) of 4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)-2-[14-oxa-2,4,10-triazatricyclo[7.5.0.0\hat{ }[3,7]]tetradeca-1(9),2,5,7-tetraen-10-yl]benzamide as a yellow solid. LC-MS: (ES, m/z): M+1=923, R,T=3.463 min. The measurements of the retention were done with a reversed phase column (C18). Shimadzu LCMS 2020; 50*3.0 Agilent Poroshell HPH-C18, 2.7 um; Eluent A: water (0.05% ammonia water); Eluent B: Acetonitrile; linear gradient from 5% acetonitrile to 95% acetonitrile in 7.0 minutes; Oven temperature 40° C.; flow: 1.5 mL/min. ¹H NMR (300 MHz, DMSO-d₆,ppm) δ11.91 (s, 1H), 11.26 (s, 1H), 8.56 (s, 1H), 8.47 (d, J=2.1 Hz, 1H), 7.61 (d, J=9.0 Hz, 1H), 7.48 (d, J=9.2 Hz, 1H), 7.37 (d, J=8.3 Hz, 2H), 7.20 (s, 1H), 7.07 (d, J=8.3 Hz, 2H), 6.99-6.83 (m, 2H), 6.76 (d, J=29.2 Hz, 2H), 6.14 (s, 1H), 4.21 (s, 2H), 3.85 (d, J=9.3 Hz, 2H), 3.52 (s, 2H), 3.30-3.14 (m, 8H), 2.79 (s, 1H), 2.23 (d, J=20.0 Hz, 5H), 1.99 (s, 4H), 1.85 (s, 1H), 1.61 (d, J=11.3 Hz, 2H), 1.42 (s, 2H), 1.25 (s, 2H), 1.03-0.79 (m, 6H). The measurements of the NMR spectra were done with Bruker AvanceIII HD 300 MHz with a probe head of BBOF.

### Compound 3-2 Preparation of 4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide

Into a 40-mL vial, was placed 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid (80 mg, 0.13 mmol, 1 equiv), DCM (3 mL), 4-[[(4-fluorooxan-4-yl)methyl]amino]-3-nitrobenzene-1-sulfonamide (42.6 mg, 0.13 mmol, 1.00 equiv), EDCI (49.0 mg, 0.26 mmol, 2 equiv), DMAP (62.4 mg, 0.51 mmol, 4 equiv). The resulting solution was stirred for 1 overnight at 25° C. The resulting mixture was concentrated under vacuum. The crude product was purified by Prep-HPLC with the following conditions (Prep-HPLC-006): Column, X Bridge Prep C18 OBD Column, 19×150 mm Sum; mobile phase, Water (10 MMOL/L NH₄HCO₃+0.1% NH₃.H₂O) and CH₃CN (41.0% CH₃CN up to 61.0% in 6 min, hold 95.0% in 1 min, down to 41.0% in 1 min, hold 41.0% in 1 min); Detector, UV 210 nm. This resulted in 17 mg (14.13%) of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[14-oxa-2,4,10-triazatricyclo[7.5.0.0\hat{ }[3,7]]tetradeca-1(9),2,5,7-tetraen-10-ylbenzamide as a yellow solid.

LC-MS: (ES, m/z): M+1=940, R,T=1.583 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 3.0 minutes; Oven temperature 40° C.; flow: 1.5 mL/min. ¹H NMR (300 MHz, DMSO-d₆,ppm) δ11.94 (s, 1H), 11.25 (s, 1H), 8.59 (s, 1H), 8.48 (d, J=2.3 Hz, 1H), 7.66 (d, J=9.1 Hz, 1H), 7.47 (d, J=8.9 Hz, 1H), 7.37 (d, J=8.2 Hz, 2H), 7.20 (d, J=3.1 Hz, 1H), 7.07 (dd, J=8.9, 3.8 Hz, 3H), 6.94 (s, 1H), 6.71 (s, 2H), 6.13 (d, J=3.1 Hz, 1H), 4.20 (d, J=6.5 Hz, 2H), 3.80-3.70 (m, 3H), 3.68-3.60 (m, 1H), 3.58-3.45 (m, 4H), 3.25-3.05 (m, 4H), 2.83-2.69 (m, 2H), 2.33-2.10 (m, 6H), 1.98 (s, 4H), 1.84-1.68 (m, 4H), 1.49-1.35 (m, 2H), 0.95 (s, 6H). The measurements of the NMR spectra were done with Bruker AvanceIII HD 300 MHz with a probe head of BBOF.

### Compound 3-3: Preparation of 4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide

Synthesis of 4,4,4-trifluoro-3-hydroxybutanamide: Into a 50-mL round-bottom flask, was placed ethyl 4,4,4-trifluoro-3-hydroxybutanoate (500 mg, 2.7 mmol, 1 equiv), NH₃ in MeOH (5 mL, 4.0 M). The resulting solution was stirred for 16 hr at 60 degrees C. The resulting mixture was concentrated. This resulted in 500 mg of 4,4,4-trifluoro-3-hydroxybutanamide as a white solid. ¹H NMR (300 MHz, DMSO-d₆, ppm) δ7.62 (ds, 1H), 7.01 (ds, 1H), 3.36-6.34 (m, 1H), 4.27-4.40 (m, 1H), 2.39-2.36 (m, 2H). The measurements of the NMR spectra were done with Bruker AvanceIII HD 300 MHz with a probe head of BBOF.

### Synthesis of 4-amino-1,1,1-trifluorobutan-2-ol: Into a 50-mL 3-necked round-bottom flask, was placed 4,4,4-trifluoro-3-hydroxybutanamide (500 mg, 3.2 mmol, 1 equiv) and THE (10 mL), LAH (242 mg, 6.4 mmol, 2.00 equiv) was added little by little at ice-bath. The resulting solution was stirred for 16 hr at R,T. After the reaction was completed, the reaction mixture was quenched by the addition of 0.24 mL of water, 0.24 mL of NaOH (10% in H₂O) and 0.72 mL of water was added into the solution successively at ice-bath. The solids were filtered out. The resulting mixture was concentrated under vacuum. This resulted in 380 mg (83.43%) of 4-amino-1,1,1-trifluorobutan-2-ol as a colorless oil. ¹HNMR (300 MHz, CDCL3, ppm) 4.10-4.01 (m, 1H), 2.70-2.66 (m, 2H), 1.51-1.47 (m, 2H). The measurements of the NMR spectra were done with Bruker AvanceIII HD 300 MHz with a probe head of BBOF

Synthesis of 4-methyl-N-(4,4,4-trifluoro-3-hydroxybutyl)benzenesulfonamide. Into a 100-mL round-bottom flask, was placed 4-amino-1,1,1-trifluorobutan-2-ol (350 mg, 2.4 mmol, 1 equiv), TEA (480 mg, 4.8 mmol, 2.0 equiv) and DCM (10 mL), TsCl (470 mg, 2.4 mmol, 1.0 equiv) was added at ice-bath. The resulting solution was stirred for 4 hr at R,T degrees C. The resulting solution was diluted with 50 mL of DCM. The resulting mixture was washed with 2×20 ml of water and 1×20 mL of brine. The mixture was dried over anhydrous sodium sulfate. The solids were filtered out. The resulting mixture was concentrated under vacuum. The residue was applied onto a silica gel column with ethyl acetate/petroleum ether (0-50%). This resulted in 600 mg (82.52%) of 4-methyl-N-(4,4,4-trifluoro-3-hydroxybutyl)benzene-1-sulfonamide as colorless oil. ¹H NMR (300 MHz, DMSO-d₆, ppm) δ7.70-7.68 (m, 2H), 7.42-7.40 (m, 2H), 6.23-6.21 (m, 1H), 4.01-3.96 (m, 1H), 2.87-2.83 (m, 2H), 2.39 (s, 3H), 1.70-1.49 (m, 2H). The measurements of the NMR spectra were done with Bruker AvanceIII HD 300 MHz with a probe head of BBOF.

Synthesis of N-(3-(5-bromo-1-((2-(trimethylsilyl)ethoxy)methyl)-1H-pyrrolo[2,3-b]pyridin-6-yloxy)-4,4,4-trifluorobutyl)-4-methylbenzenesulfonamide. Into a 100-mL round-bottom flask, was placed 5-bromo-6-fluoro-1-[[2-(trimethylsilyl)ethoxy]methyl]-1H-pyrrolo[2,3-b]pyridine (300 mg, 0.9 mmol, 1 equiv), 4-methyl-N-(4,4,4-trifluoro-3-hydroxybutyl)benzene-1-sulfonamide (310 mg, 1.0 mmol, 1.2 equiv), Cs₂CO₃ (566 mg, 1.7 mmol, 2.0 equiv) and 1,4-Dioxane (10 mL). The resulting solution was stirred for 16 hr at 90 degrees C. in an oil bath. The reaction mixture was cooled. The solids were filtered out. The resulting solution was diluted with 100 mL of DCM. The resulting mixture was washed with 5×50 ml of water and 1×50 mL of brine. The mixture was dried over anhydrous sodium sulfate. The solids were filtered out. The resulting mixture was concentrated under vacuum. The residue was applied onto a silica gel column with ethyl acetate/petroleum ether (0-30%). This resulted in 400 mg (73.95%) of N-[3-[(5-bromo-1-[[2-(trimethylsilyl)ethoxy]methyl]-1H-pyrrolo[2,3-b]pyridin-6-yl)oxy]-4,4,4-trifluorobutyl]-4-methylbenzene-1-sulfonamide as a light yellow solid. ¹H NMR (300 MHz, CDCL3, ppm) 8.17 (bs, 1H), 7.76-7.73 (m, 2H), 7.29-7.27 (m, 3H), 6.51-6.50 (m, 1H), 5.93-5.91 (m, 1H), 5.75-5.72 (m, 2H), 5.63-5.58 (m, 1H), 3.60-3.57 (m, 2H), 3.34-3.32 (m, 1H), 3.13-3.11 (m, 1H), 2.46 (s, 3H), 2.31-2.29 (m, 1H), 2.10-2.07 (m, 1H), 1.00-0.85 (m, 2H), 0.01 (s, 9H). The measurements of the NMR spectra were done with Bruker AvanceIII HD 300 MHz with a probe head of BBOF.

Synthesis of 1-tosyl-4-(trifluoromethyl)-7-((2-(trimethylsilyl)ethoxy)methyl)-1,3,4,7-tetrahydro-2H-pyrrolo[3′,2′:5,6]pyrido[2,3-b][1,4]oxazepane. Into a 250-mL round-bottom flask purged and maintained with an inert atmosphere of nitrogen, was placed N-[3-[(5-bromo-1-[[2-(trimethylsilyl)ethoxy]methyl]-1H-pyrrolo[2,3-b]pyridin-6-yl)oxy]-4,4,4-trifluorobutyl]-4-methylbenzene-1-sulfonamide (700 mg, 1.13 mmol, 1 equiv), Cs₂CO₃ (1.1 g, 3.39 mmol, 3.00 equiv), CuI (214 mg, 1.13 mmol,1.0 equiv), 2-isobutyrylcyclohexan-1-one (80 mg, 0.56 mmol, 0.5 equiv), DMSO (10 mL), The resulting solution was stirred for 24 hr at 120° C. in an oil bath. The resulting solution was diluted with 20 mL of water. The resulting solution was extracted with 2×50 mL of ethyl acetate. The resulting mixture was washed with 1×50 mL of brine. The mixture was dried over anhydrous sodium sulfate and concentrated under vacuum. The residue was applied onto a silica gel column with ethyl acetate/petroleum ether (0-30%). This resulted in 350 mg (57.38%) of 1-tosyl-4-(trifluoromethyl)-7-((2-(trimethylsilyl)ethoxy)methyl)-1,3,4,7-tetrahydro-2H-pyrrolo[3′,2′:5,6]pyrido[2,3-b][1,4]oxazepine as light yellow solid. ¹H NMR (300 MHz, CDCL3, ppm) 8.19 (bs, 1H), 7.50-7.47 (m, 2H), 7.39 (s, 1H), 7.24-7.22 (m, 2H), 6.58-6.57 (m, 1H), 5.69-5.66 (m, 1H), 5.55-5.51 (m, 1H), 4.57-4.52 (m, 1H), 3.96-3.94 (m, 1H), 3.59-3.56 (m, 2H), 3.48-3.44 (m, 1H), 2.41 (s, 3H), 2.31-2.29 (m, 1H), 1.95-1.91 (m, 1H), 0.97-0.91 (m, 2H), 0.05 (s, 9H). The measurements of the NMR spectra were done with Bruker AvanceIII HD 300 MHz with a probe head of BBOF.

Synthesis of 4-(trifluoromethyl)-7-((2-(trimethylsilyl)ethoxy)methyl)-1,3,4,7-tetrahydro-2H-pyrrolo[3′,2′:5,6]pyrido[2,3-b][1,4]oxazepane. Into a 250 mL 3-necked round-bottom flask, was placed Na (150 mg, 6.5 mmol, 1.0 equiv), naphthalene (833 mg, 6.5 mmol, 10 equiv) and DME(3 ml) under N₂. The reaction mixture was stirred at room temperature until Na and naphthalene completely dissolved. To this was added the solution 1-tosyl-4-(trifluoromethyl)-7-((2-(trimethylsilyl)ethoxy)methyl)-1,3,4,7-tetrahydro-2H-pyrrolo[3′,2′:5,6]pyrido[2,3-b][1,4]oxazepine (350 mg, 0.65 mmol, 1 equiv) in THE (5 mL) at ˜78° C. The resulting solution was stirred for 2-3 hr at ˜60° C. to ˜40° C. unstill the starting material consumed by TLC. The reaction was then quenched by the addition of 5 mL of NH₄Cl at ˜10° C. The resulting solution was extracted with 3×10 mL of ethyl acetate. The mixture was dried over anhydrous sodium sulfate and concentrated under vacuum. The residue was applied onto a silica gel column and eluted with ethyl acetate/petroleum ether (⅓). This resulted in 220 mg (88%) of 4-(trifluoromethyl)-7-((2-(trimethylsilyl)ethoxy)methyl)-1,3,4,7-tetrahydro-2H-pyrrolo[3′,2′:5,6]pyrido[2,3-b][1,4]oxazepine as a white solid.

Synthesis of methyl methyl 4-(4-((4′-chloro-5,5-dimethyl-3,4,5,6-tetrahydro-[1,1′-biphenyl]-2-yl)methyl)piperazin-1-yl)-2-(4-(trifluoromethyl)-7-((2-(trimethylsilyl)ethoxy)methyl)-3,4-dihydro-2H-pyrrolo[3′,2′:5,6]pyrido[2,3-b][1,4]oxazepin-1(7H)-yl)benzoate. Into a 100-mL round-bottom flask purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (1.2 g, 2.28 mmol, 4.00 equiv), toluene (20 mL), 4-(trifluoromethyl)-7-((2-(trimethylsilyl)ethoxy)methyl)-1,3,4,7-tetrahydro-2H-pyrrolo[3′,2′:5,6]pyrido[2,3-b][1,4]oxazepine (220 mg, 0.57 mmol, 1 equiv), Cs₂CO₃ (923 mg, 2.84 mmol, 5 equiv), XantPhos Pd 2G (250 mg, 0.46 mmol, 0.8 equiv). The resulting solution was stirred for overnight at 110° C. The resulting solution was diluted with 30 mL of water. The resulting solution was extracted with 2×30 mL of ethyl acetate. The resulting mixture was washed with 1×30 mL of brine. The mixture was dried over anhydrous sodium sulfate and concentrated under vacuum. The residue was applied onto a silica gel column with ethyl acetate/petroleum ether (0-50%). This resulted in 380 mg crude (80.0%) of methyl 4-(4-((4′-chloro-5,5-dimethyl-3,4,5,6-tetrahydro-[1,1′-biphenyl]-2-yl)methyl)piperazin-1-yl)-2-(4-(trifluoromethyl)-7-((2-(trimethylsilyl)ethoxy)methyl)-3,4-dihydro-2H-pyrrolo[3′,2′:5,6]pyrido[2,3-b][1,4]oxazepin-1(7H)-yl)benzoate as a yellow solid. LC-MS: (ES, m/z): M+H=838, R,T=3.33 min. The measurements of the retention were done with a reversed phase column (C18). Shimadzu LCMS 2020; 50*3.0, Poroshell HPH-C18, 2.7 microm; Eluent A: water (0.05% NH₄HCO₃); Eluent B: Acetonitrile; linear gradient from 5% acetonitrile to 100% acetonitrile in 5.0 minutes; Oven temperature 40° C.; flow: 1.5 mL/min.

Synthesis of methyl 4-(4-((4′-chloro-5,5-dimethyl-3,4,5,6-tetrahydro-[1,1′-biphenyl]-2-yl)methyl)piperazin-1-yl)-2-(4-(trifluoromethyl)-3,4-dihydro-2H-pyrrolo[3′,2′:5,6]pyrido[2,3-b][1,4]oxazepin-1(7H)-yl)benzoate. Into a 40-mL vial, was placed methyl 4-(4-((4′-chloro-5,5-dimethyl-3,4,5,6-tetrahydro-[1,1′-biphenyl]-2-yl)methyl)piperazin-1-yl)-2-(4-(trifluoromethyl)-7-((2-(trimethylsilyl)ethoxy)methyl)-3,4-dihydro-2H-pyrrolo[3′,2′:5,6]pyrido[2,3-b][1,4]oxazepin-1(7H)-yl)benzoate (380 mg, 0.45 mmol, 1 equiv), THE (20 mL), TBAF.3H₂O (708 mg, 2.25 mmol, 5 equiv), ethane-1,2-diamine (540 mg, 9.0 mmol, 20 equiv). The resulting solution was stirred for overnight at 70° C. in an oil bath. The resulting solution was diluted with 20 mL of water. The resulting solution was extracted with 3×30 mL of ethyl acetate. The resulting mixture was washed with 2×20 mL of brine. The mixture was dried over anhydrous sodium sulfate and concentrated under vacuum. The residue was applied onto a silica gel column with ethyl acetate/petroleum ether (0-50%). This resulted in 300 mg (93%) of methyl 4-(4-((4′-chloro-5,5-dimethyl-3,4,5,6-tetrahydro-[1,1′-biphenyl]-2-yl)methyl)piperazin-1-yl)-2-(4-(trifluoromethyl)-3,4-dihydro-2H-pyrrolo[3′,2′:5,6]pyrido[2,3-b][1,4]oxazepin-1(7H)-yl)benzoate as a light yellow solid. ¹H NMR (300 MHz, CDCL3, ppm) 10.78 (bs, 1H), 7.75-7.72 (m, 1H), 7.29-7.24 (m, 3H), 7.00-6.97 (m, 2H), 6.59-6.56 (m, 2H), 6.28-6.26 (m, 1H), 5.28-5.20 (m, 1H), 3.98-3.96 (m, 1H), 3.79-3.77 (m, 1H), 3.59 (s, 1H), 3.24-3.23 (m, 3H), 2.84 (m, 2H), 2.31-2.28 (m, 4H), 2.26-2.24 (m, 4H), 1.49-1.47 (m, 2H), 1.34-1.24 (m, 4H), 1.00 (s, 6H), 0.96-0.90 (m, 2H).

Synthesis of 4-(4-((4′-chloro-5,5-dimethyl-3,4,5,6-tetrahydro-[1,1′-biphenyl]-2-yl)methyl)piperazin-1-yl)-2-(4-(trifluoromethyl)-3,4-dihydro-2H-pyrrolo[3′,2′:5,6]pyrido[2,3-b][1,4]oxazepin-1(7H)-yl)benzoic acid. Into a 40-mL vial, was placed methyl 4-(4-((4′-chloro-5,5-dimethyl-3,4,5,6-tetrahydro-[1,1′-biphenyl]-2-yl)methyl)piperazin-1-yl)-2-(4-(trifluoromethyl)-3,4-dihydro-2H-pyrrolo[3′,2′:5,6]pyrido[2,3-b][1,4]oxazepin-1(7H)-yl)benzoate (200 mg, 0.28 mmol, 1 equiv), MeOH (6 mL), 1,4-dioxane (6 mL), H₂O (2 mL), NaOH (67 mg, 1.68 mmol, 6.00 equiv). The resulting solution was stirred for overnight at 60° C. in an oil bath. The resulting mixture was concentrated under vacuum. The pH value of the solution was adjusted to 5-6 with HCl (2 mol/L). The resulting solution was extracted with 2×50 mL of dichloromethane/MeOH (v:v=10:1). The resulting mixture was washed with 2×200 mL of brine. The mixture was dried over anhydrous sodium sulfate and concentrated under vacuum. This resulted in 157 mg (81.0%) of 4-(4-((4′-chloro-5,5-dimethyl-3,4,5,6-tetrahydro-[1,1′-biphenyl]-2-yl)methyl)piperazin-1-yl)-2-(4-(trifluoromethyl)-3,4-dihydro-2H-pyrrolo[3′,2′:5,6]pyrido[2,3-b][1,4]oxazepin-1(7H)-yl)benzoic acid as a light yellow solid. LC-MS: (ES, m/z): M+H=694, R,T=2.43 min. The measurements of the retention were done with a reversed phase column (C18). Shimadzu LCMS 2020; 50*3.0, Poroshell HPH-C18, 2.7 microm; Eluent A: water (0.05% NH₄HCO₃); Eluent B: Acetonitrile; linear gradient from 5% acetonitrile to 100% acetonitrile in 5.0 minutes; Oven temperature 40° C.; flow: 1.5 mL/min.

Synthesis of 4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide. Into a 40-mL round-bottom flask, was placed 4-(4-((4′-chloro-5,5-dimethyl-3,4,5,6-tetrahydro-[1,1′-biphenyl]-2-yl)methyl)piperazin-1-yl)-2-(4-(trifluoromethyl)-3,4-dihydro-2H-pyrrolo[3′,2′:5,6]pyrido[2,3-b][1,4]oxazepin-1(7H)-yl)benzoic acid (57 mg, 0.08 mmol, 1 equiv), DCM (3 mL), 3-nitro-4-[[(oxan-4-yl)methyl]amino]benzene-1-sulfonamide (25.2 mg, 0.08 mmol, 1.00 equiv), EDCI (30.6 mg, 0.16 mmol, 2 equiv), DMAP (39.0 mg, 0.32 mmol, 4 equiv). The resulting solution was stirred for overnight at 25 degrees C. The resulting mixture was concentrated under vacuum. The residue was applied onto a silica gel column with dichloromethane/methanol (10:1). The crude product was purified by Flash-Prep-HPLC with the following conditions (IntelFlash-1): Column, C18 reversed phase column; mobile phase, Water (10MMOL/L NH₄HCO₃+0.05% NH₃.H₂O) and CH₃CN (20.0% CH₃CN up to 90.0% in 30 min); Detector, UV 220 nm. This resulted in 32 mg (40.0%) of 4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide as a yellow solid. LC-MS: (ES, m/z): M+1=991, R,T=2.41 min. The measurements of the retention were done with a reversed phase column (C18). Shimadzu LCMS 2020; 50*3.0 Agilent Poroshell HPH-C18, 2.7 um; Eluent A: water (0.05% ammonia water); Eluent B: Acetonitrile; linear gradient from 5% acetonitrile to 95% acetonitrile in 7.0 minutes; Oven temperature 40° C.; flow: 1.5 mL/min. ¹H NMR (300 MHz, CDCL3, ppm) δ11.43 (ds, 1H), 9.99 (ds, 1H), 8.72 (s, 1H), 8.40 (ds, 1H), 7.93-7.85 (m, 2H), 7.37-7.35 (m, 2H), 7.26 (s, 1H), 7.02-6.93 (m, 3H), 6.71-6..64 (m, 3H), 6.27 (s, 1H), 4.58 (s, 1H), 4.05-3.15 (m, 23H), 2.70-2.35 (m, 7H), 2.03 (s, 3H), 1.92-1.90 (m, 1H), 1.89-1.87 (m, 2H), 1.71-1.67 (m, 2H), 1.53-1.45 (m, 3H), 1.00 (s, 6H). The measurements of the NMR spectra were done with Bruker AvanceIII HD 300 MHz with a probe head of BBOF.

### Compound 3-4: Preparation of 4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]thiazepin-1(7H)-yl)-N-((3-nitro-4-(((tetrahydro-2H-pyran-4-yl)methyl)amino)phenyl)sulfonyl)benzamide

Synthesis of methyl 3-[(5-bromo-1-[[2-(trimethylsilyl)ethoxy]methyl]-1H-pyrrolo[2,3-b]pyridin-6-yl)sulfanyl]propanoate. Into a 40-mL round-bottom flask, was placed 5-bromo-6-fluoro-1-[[2-(trimethylsilyl)ethoxy]methyl]-1H-pyrrolo[2,3-b]pyridine (3 g, 8.7 mmol, 1 equiv), methyl 3-sulfanylpropanoate (2.1 g, 17.4 mmol, 2.0 equiv), ACN (30 mL), Cs₂CO₃ (7.1 g, 21.7 mmol, 2.5 equiv). The resulting solution was stirred for 1 hr at 70° C.

The solids were filtered out. The resulting mixture was concentrated. The residue was applied onto a silica gel column with ethyl acetate/petroleum ether (0-10%). This resulted in 300 mg (7.75%) of methyl 3-[(5-bromo-1-[[2-(trimethylsilyl)ethoxy]methyl]-1H-pyrrolo[2,3-b]pyridin-6-yl)sulfanyl]propanoate as a colorless oil. LC-MS: (ES, m/z): M+1=445/447, R,T=1.47 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Synthesis of 3-[(5-bromo-1-[[2-(trimethylsilyl)ethoxy]methyl]-1H-pyrrolo[2,3-b]pyridin-6-yl)sulfanyl]propan-1-ol. Into a 8-mL round-bottom flask purged and maintained with an inert atmosphere of nitrogen, was placed methyl 3-[(5-bromo-1-[[2-(trimethylsilyl)ethoxy]methyl]-1H-pyrrolo[2,3-b]pyridin-6-yl)sulfanyl]propanoate (300 mg, 0.67 mmol, 1 equiv) and THE (10 mL). This was followed by the addition of LiAlH₄ (51 mg, 1.3 mmol, 2.00 equiv) at ˜78° C. carefully. The resulting solution was stirred for 1 hr at room temperature. After the reaction was completed, the reaction mixture was quenched by the addition of 0.5 mL of water, 0.5 mL of NaOH (10% in H₂O) and 1.5 mL of water was added into the solution successively at ice-bath. The solids were filtered out. The resulting mixture was concentrated under vacuum. This resulted in 200 mg (71.14%) of 3-[(5-bromo-1-[[2-(trimethylsilyl)ethoxy]methyl]-1H-pyrrolo[2,3-b]pyridin-6-yl)sulfanyl]propan-1-ol as yellow oil. H-NMR: (CDCl₃,300 ppm): 7.99 (s, 1H), 7.24-7.20 (m, 1H), 6.49-6.41 (m, 1H), 5.32 (s, 2H), 3.84-3.78 (m, 2H), 3.58-3.50 (m, 2H), 3.46-3.40 (m, 2H), 2.09-2.01 (m, 2H), 0.96-0.88 (m, 2H), 0.05 (s, 9H).

### Synthesis of N-[6-[(3-hydroxypropyl)sulfanyl]-1-[[2-(trimethylsilyl)ethoxy]methyl]-1H-pyrrolo[2,3-b]pyridin-5-yl]-4-methylbenzene-1-sulfonamide. Into a 100-mL round-bottom flask purged and maintained with an inert atmosphere of nitrogen was placed 4-methylbenzene-1-sulfonamide (1.6 g, 9.6 mmol, 2 equiv), 3-[(5-bromo-1-[[2-(trimethylsilyl)ethoxy]methyl]-1H-pyrrolo[2,3-b]pyridin-6-yl)sulfanyl]propan-1-ol (2 g, 4.8 mmol, 1 equiv), 1,10-phenanthroline (0.17 g, 0.94 mmol, 0.20 equiv), CuI (0.18 g, 0.96 mmol, 0.2 equiv), Cs₂CO₃ (3.1 g, 9.6 mmol, 2 equiv) under DMSO (30 mL). The resulting solution was stirred for 72 hr at 120° C. The resulting solution was diluted with 30 mL of H₂O. The solids were filtered out. The resulting solution was extracted with 3×50 mL of ethyl acetate, The mixture was dried over anhydrous sodium sulfate and concentrated under vacuum. The residue was applied onto a silica gel column with ethyl acetate/petroleum ether (0:1-1:1). This resulted in 1.0 g (41.11%) of N-[6-[(3-hydroxypropyl)sulfanyl]-1-[[2-(trimethylsilyl)ethoxy]methyl]-1H-pyrrolo[2,3-b]pyridin-5-yl]-4-methylbenzene-1-sulfonamide as a white solid. LC-MS: (ES, m/z): M+1=508, R,T=2.845 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient

Synthesis of 10-(4-methylbenzenesulfonyl)-4-[[2-(trimethylsilyl)ethoxy]methyl]-14-thia-2,4,10-triazatricyclo[7.5.0.0\hat{ }[3,7]]tetradeca-1(9),2,5,7-tetraene. Into a 8-mL round-bottom flask, was placed N-[6-[(3-hydroxypropyl)sulfanyl]-1-[[2-(trimethylsilyl)ethoxy]methyl]-1H-pyrrolo[2,3-b]pyridin-5-yl]-4-methylbenzene-1-sulfonamide (240 mg, 0.47 mmol, 1 equiv), THE (5 ml), PPh₃ (248 mg, 0.95 mmol, 2 equiv). This was followed by the addition of DEAD (164 mg, 0.95 mmol, 2.00 equiv) dropwise with stirring at 0° C. The resulting solution was stirred for 12 hrs at room temperature. The resulting mixture was concentrated. The residue was applied onto a silica gel column with ethyl acetate/petroleum ether (0:1-1:2). This resulted in 200 mg (86.40%) of 10-(4-methylbenzenesulfonyl)-4-[[2-(trimethylsilyl)ethoxy]methyl]-14-thia-2,4,10-triazatricyclo[7.5.0.0\hat{ }[3,7]]tetradeca-1(9),2,5,7-tetraene as a colorless solid. H-NMR:(CDCl₃,300 ppm): 7.82 (s, 1H), 7.69-7.58 (m, 3H), 7.38-7.36 (d, J=6 Hz, 2H), 6.55-6.54 (d, J=3 Hz, 2H), 5.55 (s, 2H), 4.30-4.23 (m, 2H)4.06-4.02 (m, 2H), 3.57-3.49 (m, 2H), 2.77 (s, 2H), 2.50 (s, 3H), 2.07-1.99 (m, 3H), 1.24-1.04 (m, 9H), 0.87-0.84 (m, 3H), 0.02 (s, 9H).

Synthesis of 4-[[2-(trimethylsilyl)ethoxy]methyl]-14-thia-2,4,10-triazatricyclo[7.5.0.0\hat{ }[3,7]]tetradeca-1(9),2,5,7-tetraene. Into a 8-mL round-bottom flask, was placed naphthalene (314 mg, 2.4 mmol, 6 equiv), Na (90 mg, 3.9 mmol, 9.6 equiv) and DME (5 mL), The resulting solution was stirred for 0.5 hr at room temperature. The resulting solution was added to 40-mL round-bottom flask of 10-(4-methylbenzenesulfonyl)-4-[[2-(trimethylsilyl)ethoxy]methyl]-14-thia-2,4,10-triazatricyclo[7.5.0.0\hat{ }[3,7]]tetradeca-1(9),2,5,7-tetraene (200 mg, 0.41 mmol, 1 equiv), THE (10 mL). The resulting solution was stirred for 3 hr at room temperature. The reaction was then quenched by the addition of 1 mL of NH₄Cl. The resulting mixture was concentrated. The residue was applied onto a silica gel column with ethyl acetate/petroleum ether (0-50%). This resulted in 120 mg (87.57%) of 4-[[2-(trimethylsilyl)ethoxy]methyl]-14-thia-2,4,10-triazatricyclo[7.5.0.0\hat{ } [3,7]]tetradeca-1(9),2,5,7-tetraene as a yellow solid. LC-MS:(ES, m/z): M+1=649, R,T=0.80 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Synthesis of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-thia-2,4,10-triazatricyclo[7.5.0.0\hat{ }[3,7]]tetradeca-1(9),2,5,7-tetraen-10-yl)benzoate. Into a 8-mL round-bottom flask purged and maintained with an inert atmosphere of nitrogen, was placed 4-[[2-(trimethylsilyl)ethoxy]methyl]-14-thia-2,4,10-triazatricyclo[7.5.0.0\hat{ }[3,7]]tetradeca-1(9),2,5,7-tetraene (120 mg, 0.36 mmol, 1 equiv), methyl 2-bromo-4-(4-[[2-(4-chlorophenyl)-4,4-dimethylcyclohex-1-en-1-yl]methyl]piperazin-1-yl)benzoate (380 mg, 0.71 mmol, 2.00 equiv), caesio methaneperoxoate caesium (233 mg, 0.71 mmol, 2.00 equiv), toluene (3 mL), XantPhosPd (34 mg, 0.04 mmol, 0.1 equiv). The resulting solution was stirred for overnight at 110° C. The resulting mixture was concentrated. The residue was applied onto a silica gel column with ethyl acetate/petroleum ether (0-50%). This resulted in 150 mg (53.32%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-thia-2,4,10-triazatricyclo[7.5.0.0\hat{ }[3,7]]tetradeca-1(9),2,5,7-tetraen-10-yl)benzoate as a yellow solid. LC-MS: (ES, m/z): M+1=786, R,T=1.26 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient. H-NMR:(CDCl₃,300 ppm):7.64-7.61 (d, J=9 Hz, 1H), 7.35 (s, 1H), 7.28-7.26 (m, 2H), 7.19-7.18 (m, 1H), 6.99-6.97 (m, 2H), 6.52-6.47 (m, 2H), 6.27-6.26 (d, J=3 Hz, 1H), 5.61 (s, 2H), 3.96-3.84 (m, 2H), 3.60-3.54 (m, 5H), 3.28-3.16 (m, 6H), 2.82 (s, 2H), 2.33-2.24 (m, 6H), 2.06-2.02 (m, 4H), 1.49-1.45 (m, 2H), 1.28-1.19 (m, 4H), 1.03-0.88 (m, 6H), 0.00 (s, 9H).

Synthesis of methyl 4-(4-[[2-(4-chlorophenyl)-4,4-dimethylcyclohex-1-en-1-yl]methyl]piperazin-1-yl)-2-[14-thia-2,4,10-triazatricyclo[7.5.0.0\hat{ }[3,7]]tetradeca-1(9),2,5,7-tetraen-10-yl]benzoate. Into a 8-mL round-bottom flask, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-thia-2,4,10-triazatricyclo[7.5.0.0\hat{ }[3,7]]tetradeca-1(9),2,5,7-tetraen-10-yl)benzoate (150 mg, 0.19 mmol, 1 equiv), ethane-1,2-diamine (229 mg, 3.8 mmol, 20 equiv), TBAF (997 mg, 3.8 mmol, 20 equiv), THE (10 mL). The resulting solution was stirred for 14 hr at 70° C. The resulting mixture was concentrated. The residue was applied onto a silica gel column with ethyl acetate/petroleum ether (0-50%). This resulted in 50 mg (39.95%) of methyl 4-(4-[[2-(4-chlorophenyl)-4,4-dimethylcyclohex-1-en-1-yl]methyl]piperazin-1-yl)-2-[14-thia-2,4,10-triazatricyclo[7.5.0.0\hat{ }[3,7]]tetradeca-1(9),2,5,7-tetraen-10-yl]benzoate as a yellow solid. LC-MS: (ES, m/z): M+1=656, R,T=1.05 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Synthesis of 4-(4-[[2-(4-chlorophenyl)-4,4-dimethylcyclohex-1-en-1-yl]methyl]piperazin-1-yl)-2-[14-thia-2,4,10-triazatricyclo[7.5.0.0\hat{ }[3,7]]tetradeca-1(9),2,5,7-tetraen-10-yl]benzoic acid. Into a 8-mL round-bottom flask, was placed methyl 4-(4-[[2-(4-chlorophenyl)-4,4-dimethylcyclohex-1-en-1-yl]methyl]piperazin-1-yl)-2-[14-thia-2,4,10-triazatricyclo[7.5.0.0\hat{ }[3,7]]tetradeca-1(9),2,5,7-tetraen-10-yl]benzoate (65 mg, 0.10 mmol, 1 equiv), 1,4-dioxane (1 mL), H₂O (1 mL), NaOH (23.95 mg, 0.60 mmol, 6.00 equiv). The resulting solution was stirred for 14 hr at 90° C. The pH value of the solution was adjusted to 5 with HCl (2 mol/L). The resulting mixture was concentrated. The residue was applied onto a silica gel column with dichloromethane/methanol (0-10%). This resulted in 30 mg (46.80%) of 4-(4-[[2-(4-chlorophenyl)-4,4-dimethylcyclohex-1-en-1-yl]methyl]piperazin-1-yl)-2-[14-thia-2,4,10-triazatricyclo[7.5.0.0\hat{ }[3,7]]tetradeca-1(9),2,5,7-tetraen-10-yl]benzoic acid as a yellow solid.

Synthesis of 4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)-2-[14-thia-2,4,10-triazatricyclo[7.5.0.0\hat{ }[3,7]]tetradeca-1(9),2,5,7-tetraen-10-yl]benzamide. Into a 8-mL round-bottom flask, was placed 4-(4-[[2-(4-chlorophenyl)-4,4-dimethylcyclohex-1-en-1-yl]methyl]piperazin-1-yl)-2-[14-thia-2,4,10-triazatricyclo[7.5.0.0\hat{ }[3,7]]tetradeca-1(9),2,5,7-tetraen-10-yl]benzoic acid (30 mg, 0.05 mmol, 1 equiv), 3-nitro-4-[[(oxan-4-yl)methyl]amino]benzene-1-sulfonamide (17 mg, 0.06 mmol, 1.20 equiv), EDCI (18 mg, 0.09 mmol, 2 equiv), DMAP (23 mg, 0.19 mmol, 4 equiv), DCM (3 mL). The resulting solution was stirred for overnight at room temperature. The resulting mixture was concentrated. The crude product was purified by Flash-Prep-HPLC with the following conditions (IntelFlash-1): Column, C18 silica gel; mobile phase, Water(0.1% FA) and ACN (48.0% ACN up to 53.0% in 7 min, hold 95.0% in 1 min, down to 48.0% in 1 min within 5; Detector, UV 254 nm. This resulted in 10.6 mg (24.15%) of 4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)-2-[14-thia-2,4,10-triazatricyclo[7.5.0.0\hat{ }[3,7]]tetradeca-1(9),2,5,7-tetraen-10-yl]benzamide as a yellow solid. LC-MS: (ES, m/z): M+1=939, R,T=3.55 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient. H-NMR: (CDCl₃,300 ppm): 8.71 (s, 1H), 8.49 (s, 1H), 7.99-7.97 (m, 1H), 7.81-7.77 (m, 1H), 7.38-7.28 (m, 4H), 7.01-6.93 (m, 2H), 6.88-6.72 (m, 3H), 3.36 (s, 1H), 4.06-3.26 (m, 18H), 2.73-2.22 (m, 6H), 2.13-1.73 (m, 3H), 1.79-1.25 (m, 6H), 1.00 (s, 6H).

### Compound 3-5: Preparation of 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide

Synthesis of methyl 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]benzoate: Into a 8-mL round-bottom flask, was placed methyl 4-(4-[[2-(4-chlorophenyl)-4,4-dimethylcyclohex-1-en-1-yl]methyl]piperazin-1-yl)-2-[14-thia-2,4,10-triazatricyclo[7.5.0.0\hat{ }[3,7]]tetradeca-1(9),2,5,7-tetraen-10-yl]benzoate (30 mg, 0.046 mmol, 1 equiv), DCM (3 mL), m-CPBA (19.72 mg, 0.114 mmol, 2.50 equiv). The resulting solution was stirred for overnight at room temperature. The reaction was then quenched by the addition of 1 mL of water. The resulting solution was extracted with 2×5 mL of dichloromethane concentrated. The residue was applied onto a silica gel column with ethyl acetate/petroleum ether (0:1-1:1). This resulted in 20 mg (63.57%) of methyl 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]benzoate as a white solid. LC-MS-PH-PHNW-4-65-8: (ES, m/z): M+1=688, R,T=0.967 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Synthesis of 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]benzoic acid. Into a 8-mL round-bottom flask, was placed methyl 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]benzoate(20 mg, 0.029 mmol, 1 equiv), 1,4-dioxane (1 mL), water (1 mL), NaOH(6.97 mg, 0.174 mmol, 6.00 equiv). The resulting solution was stirred for overnight at 70° C. The pH value of the solution was adjusted to 5 with HCl (1 mol/L). The resulting solution was extracted with 2×3 mL of ethyl acetate. The organic layer was washed with 2×3 ml of Brine. The mixture was dried over anhydrous sodium sulfate and concentrated. This resulted in 13 mg (66.35%) of 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]benzoic acid as a white solid. LC-MS-PH-PHNW-4-65-9: (ES, m/z): M+1=674, R,T=1.945 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

### Synthesis of 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide. Into a 8-mL round-bottom flask, was placed 3-nitro-4-[[(oxan-4-yl)methyl]amino]benzene-1-sulfonamide (6.08 mg, 0.019 mmol, 1 equiv), 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]benzoic acid (13 mg, 0.019 mmol, 1 equiv), DCM (0.47 mL, 5.506 mmol, 381.56 equiv), DMAP (9.42 mg, 0.077 mmol, 4 equiv), EDCI (7.39 mg, 0.039 mmol, 2 equiv). The resulting solution was stirred for overnight at room temperature. The resulting mixture was concentrated. The crude product was further purified by Prep-HPLC with the following conditions (Waters I): Column, Xbridge Prep C18 OBD column, Sum, 19*150 mm; mobile phase, Water (0.05% TFA) and CH₃CN (46% CH₃CN up to 51% in 7 min); Detector, UV 220&254 nm. This resulted in 2.5 mg (6.24%) of 4-(4-[[2-(4-chlorophenyl)-4,4-dimethylcyclohex-1-en-1-yl]methyl]piperazin-1-yl)-2-[14,14-dioxo-141ambda6-thia-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide as a yellow solid. LC-MS-PH-PHNW-4-65-0: (ES, m/z): M+1=971.5, R,T=3.243 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient

### Compound 3-6: Preparation of 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide

Synthesis of 2-methoxypropane-1,3-diol. Into a 1000-mL 3-necked round-bottom flask purged and maintained with an inert atmosphere of nitrogen, was placed 1,3-dimethyl 2-methoxypropanedioate (20 g, 123 mmol, 1 equiv) and THE (250 mL). LiAlH₄ (23.4 g, 616 mmol, 5.0 equiv) was added little by little at ice-bath. The resulting solution was stirred for 16 hr at R,T. After the reaction was completed, the reaction mixture was quenched by the addition of 23.4 mL of water, 23.4 mL of NaOH (10% in H₂O) and 70 mL of water was added into the solution successively at ice-bath. The solids were filtered out. The resulting mixture was concentrated under vacuum. This resulted in 12 g (91.67%) of 2-methoxypropane-1,3-diol as colorless oil. ¹H NMR (300 MHz, CDCL3, ppm) δ3.80-3.63 (m, 4H), 3.47 (s, 3H), 3.36-3.32 (m, 1H), 3.25 (bs, 2H).

Synthesis of 3-(5-bromo-1-((2-(trimethylsilyl)ethoxy)methyl)-1H-pyrrolo[2,3-b]pyridin-6-yloxy)-2-methoxypropan-1-ol. I Into a 250-mL 3-necked round-bottom flask, was placed 2-methoxypropane-1,3-diol (1.84 g, 17.4 mmol, 1.2 equiv) and THF (70 mL), NaH (0.87 g, 36.2 mmol, 2.5 equiv) was added at ice-bath, after the mixture was stirred at R,T for 30 min, and then 5-bromo-6-fluoro-1-[[2-(trimethylsilyl)ethoxy]methyl]-1H-pyrrolo[2,3-b]pyridine (5.0 g, 14.5 mmol, 1 equiv) was added at R,T. The resulting solution was stirred for 4 hr at 80 degrees C. The reaction was then quenched by the addition of 20 mL of water. The resulting solution was extracted with 3×50 mL of dichloromethane and the organic layers combined. The resulting mixture was washed with 1×50 mL of brine. The mixture was dried over anhydrous sodium sulfate. The solids were filtered out. The resulting mixture was concentrated. The residue was applied onto a silica gel column with ethyl acetate/petroleum ether (0-40%). This resulted in 4.0 g (64.03%) of 3-[(5-bromo-1-[[2-(trimethylsilyl)ethoxy]methyl]-1H-pyrrolo[2,3-b]pyridin-6-yl)oxy]-2-methoxypropan-1-ol as a white solid. ¹H NMR (300 MHz, CDCL3, ppm) 8.09 (s, 1H), 7.20 (d, J=3.6 Hz, 1H), 6.44 (d, J=3.6 Hz, 1H), 5.62 (s, 2H), 4.60-4.58 (m, 2H), 3.97-3.95 (m, 1H), 3.92-3.90 (m, 2H), 3.79 (s, 3H), 3.65-3.60 (m, 2H), 0.98-0.93 (m, 2H), 0.01 (s, 9H).

### Synthesis of N-(6-(3-hydroxy-2-methoxypropoxy)-1-((2-(trimethylsilyl)ethoxy)methyl)-1H-pyrrolo[2,3-b]pyridin-5-yl)-4-methylbenzenesulfonamide

Into a 250-mL 3-necked round-bottom flask purged and maintained with an inert atmosphere of nitrogen, was placed 3-[(5-bromo-1-[[2-(trimethylsilyl)ethoxy]methyl]-1H-pyrrolo[2,3-b]pyridin-6-yl)oxy]-2-methoxypropan-1-ol (4.0 g, 9.3 mmol, 1 equiv), Ts-NH₂ (4.76 g, 27.8 mmol, 3.00 equiv), Cs₂CO₃ (9.06 g, 27.8 mmol, 3.0 equiv), CuI (0.88 g, 4.6 mmol, 0.5 equiv), 1,10-phenanthroline (0.50 g, 2.8 mmol, 0.3 equiv) and DMSO (100 mL). The resulting solution was stirred for 24 hr at 120 degrees C. in an oil bath. The reaction mixture was cooled. The resulting solution was diluted with 500 mL of DCM. The solids were filtered out. The mixture was dried over anhydrous sodium sulfate. The solids were filtered out. The resulting mixture was concentrated. The residue was applied onto a silica gel column with ethyl acetate/petroleum ether (0-60%). This resulted in 2.7 g (55.82%) of N-[6-(3-hydroxy-2-methoxypropoxy)-1-[[2-(trimethylsilyl)ethoxy]methyl]-1H-pyrrolo[2,3-b]pyridin-5-yl]-4-methylbenzene-1-sulfonamide as a solid. ¹H NMR (300 MHz, CDCL3, ppm) 8.09 (s, 1H), 7.61-7.59 (m, 2H), 7.17-7.15 (m, 3H), 6.75 (s, 1H), 6.46 (d, J=3.3 Hz, 1H), 5.48 (s, 2H), 4.28-4.23 (m, 2H), 3.59-3.44 (m, 8H), 2.37 (s, 3H), 0.92-0.86 (m, 3H), 0.01 (s, 9H).

Synthesis of 3-methoxy-1-tosyl-7-((2-(trimethylsilyl)ethoxy)methyl)-1,3,4,7-tetrahydro-2H-pyrrolo[3′,2′:5,6]pyrido[2,3-b][1,4]oxazepane. Into a 100-mL 3-necked round-bottom flask purged and maintained with an inert atmosphere of nitrogen, was placed N-[6-(3-hydroxy-2-methoxypropoxy)-1-[[2-(trimethylsilyl)ethoxy]methyl]-1H-pyrrolo[2,3-b]pyridin-5-yl]-4-methylbenzene-1-sulfonamide (500 mg, 0.96 mmol, 1 equiv), PPh₃ (1.2 g, 4.79 mmol, 5.0 equiv) and THE (10 mL), DEAD (834 mg, 4.79 mmol, 5.0 equiv) was added dropwise at ice-bath. The resulting solution was stirred for 2 hr at R,T. The resulting solution was diluted with 50 mL of DCM. The resulting mixture was washed with 3×20 ml of water and 1×20 mL of brine. The mixture was dried over anhydrous sodium sulfate. The solids were filtered out. The resulting mixture was concentrated under vacuum. The residue was applied onto a silica gel column with ethyl acetate/petroleum ether (0-60%). This resulted in 370 mg (76.65%) of 3-methoxy-1-tosyl-7-((2-(trimethylsilyl)ethoxy)methyl)-1,3,4,7-tetrahydro-2H-pyrrolo[3′,2′:5,6]pyrido[2,3-b][1,4]oxazepine as a white solid. LC-MS: (ES, m/z): M+H=504, R,T=2.40 min.

Synthesis of 3-methoxy-7-((2-(trimethylsilyl)ethoxy)methyl)-1,3,4,7-tetrahydro-2H-pyrrolo[3′,2′:5,6]pyrido[2,3-b][1,4]oxazepane. Into a 250 mL 3-necked round-bottom flask, was placed Na (158 mg, 6.9 mmol, 1.0 equiv), naphthalene (884 mg, 6.9 mmol, 10 equiv) and DME(3 ml) under N₂. The reaction mixture was stirred at room temperature until Na and naphthalene completely dissolved. To this was added the solution 3-methoxy-1-tosyl-7-((2-(trimethylsilyl)ethoxy)methyl)-1,3,4,7-tetrahydro-2H-pyrrolo[3′,2′:5,6]pyrido[2,3-b][1,4]oxazepine (350 mg, 0.69 mmol, 1 equiv) in THE (5 mL) at ˜78° C. The resulting solution was stirred for 2-3 hr at ˜60° C. to ˜40° C. unstill the starting material consumed by TLC. The reaction was then quenched by the addition of 5 mL of NH₄Cl at ˜10° C. The resulting solution was extracted with 3×10 mL of ethyl acetate. The mixture was dried over anhydrous sodium sulfate and concentrated under vacuum. The residue was applied onto a silica gel column and eluted with ethyl acetate/petroleum ether (⅓). This resulted in 214 mg (88%) of 3-methoxy-7-((2-(trimethylsilyl)ethoxy)methyl)-1,3,4,7-tetrahydro-2H-pyrrolo[3′,2′:5,6]pyrido[2,3-b][1,4]oxazepine as a white solid. LC-MS: (ES, m/z): M+H=350, R,T=2.26 min.

Synthesis of methyl 4-(4-((4′-chloro-5,5-dimethyl-3,4,5,6-tetrahydro-[1,1′-biphenyl]-2-yl)methyl)piperazin-1-yl)-2-(3-methoxy-7-((2-(trimethylsilyl)ethoxy)methyl)-3,4-dihydro-2H-pyrrolo[3′,2′:5,6]pyrido[2,3-b][1,4]oxazepin-1(7H)-yl)benzoate. Into a 100-mL round-bottom flask purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (1.34 g, 2.52 mmol, 4.00 equiv), toluene (20 mL), 3-methoxy-7-((2-(trimethylsilyl)ethoxy)methyl)-1,3,4,7-tetrahydro-2H-pyrrolo[3′,2′:5,6]pyrido[2,3-b][1,4]oxazepine (220 mg, 0.63 mmol, 1 equiv), Cs₂CO₃ (1.02 g, 3.15 mmol, 5 equiv), XantPhos Pd 2G (25 mg, 0.06 mmol, 0.1 equiv). The resulting solution was stirred for overnight at 110° C. The resulting solution was diluted with 30 mL of water. The resulting solution was extracted with 2×30 mL of ethyl acetate. The resulting mixture was washed with 1×30 mL of brine. The mixture was dried over anhydrous sodium sulfate and concentrated under vacuum. The residue was applied onto a silica gel column with ethyl acetate/petroleum ether (0-50%). This resulted in 403 mg crude (80.0%) of methyl 4-(4-((4′-chloro-5,5-dimethyl-3,4,5,6-tetrahydro-[1,1′-biphenyl]-2-yl)methyl)piperazin-1-yl)-2-(3-methoxy-7-((2-(trimethylsilyl)ethoxy)methyl)-3,4-dihydro-2H-pyrrolo[3′,2′:5,6]pyrido[2,3-b][1,4]oxazepin-1(7H)-yl)benzoate as a yellow solid.

Synthesis of methyl 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)benzoate. Into a 40-mL vial, was placed methyl 4-(4-((4′-chloro-5,5-dimethyl-3,4,5,6-tetrahydro-[1,1′-biphenyl]-2-yl)methyl)piperazin-1-yl)-2-(3-methoxy-7-((2-(trimethylsilyl)ethoxy)methyl)-3,4-dihydro-2H-pyrrolo[3′,2′:5,6]pyrido[2,3-b][1,4]oxazepin-1(7H)-yl)benzoate (380 mg, 0.48 mmol, 1 equiv), THE (20 mL), TBAF.3H₂O (756 mg, 2.4 mmol, 5 equiv), ethane-1,2-diamine (576 mg, 9.6 mmol, 20 equiv). The resulting solution was stirred for overnight at 70° C. in an oil bath. The resulting solution was diluted with 20 mL of water. The resulting solution was extracted with 3×30 mL of ethyl acetate. The resulting mixture was washed with 2×20 mL of brine. The mixture was dried over anhydrous sodium sulfate and concentrated under vacuum. The residue was applied onto a silica gel column with ethyl acetate/petroleum ether (0-50%). This resulted in 338 mg (93%) of methyl 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)benzoate as a light yellow solid. LC-MS: (ES, m/z): M+H=670, R,T=2.00 min.

Synthesis of 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)benzoic acid. Into a 40-mL vial, was placed methyl 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)benzoate (200 mg, 0.30 mmol, 1 equiv), MeOH (6 mL), 1,4-dioxane (6 mL), H₂O (2 mL), NaOH (72 mg, 1.8 mmol, 6.00 equiv). The resulting solution was stirred for overnight at 60° C. in an oil bath. The resulting mixture was concentrated under vacuum. The pH value of the solution was adjusted to 5-6 with HCl (2 mol/L). The resulting solution was extracted with 2×50 mL of dichloromethane/MeOH (v:v=10:1). The resulting mixture was washed with 2×200 mL of brine. The mixture was dried over anhydrous sodium sulfate and concentrated under vacuum. This resulted in 152 mg (81.0%) of 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)benzoic acid as a light yellow solid. LC-MS: (ES, m/z): M+H=656, R,T=2.43 min.

### Synthesis of 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide. Into a 40-mL round-bottom flask, was placed 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)benzoic acid (50 mg, 0.08 mmol, 1 equiv), DCM (3 mL), 3-nitro-4-[[(oxan-4-yl)methyl]amino]benzene-1-sulfonamide (25.2 mg, 0.08 mmol, 1.00 equiv), EDCI (30.6 mg, 0.16 mmol, 2 equiv), DMAP (39.0 mg, 0.32 mmol, 4 equiv). The resulting solution was stirred for overnight at 25 degrees C. The resulting mixture was concentrated under vacuum. The residue was applied onto a silica gel column with dichloromethane/methanol (10:1). The crude product was purified by Flash-Prep-HPLC with the following conditions (IntelFlash-1): Column, C18 reversed phase column; mobile phase, Water (10 MMOL/L NH₄HCO₃+0.05% NH₃.H₂O) and CH₃CN (20.0% CH₃CN up to 90.0% in 30 min); Detector, UV 220 nm. This resulted in 32 mg (40.0%) of 4-(4-((4′-chloro-5,5-dimethyl-3,4,5,6-tetrahydro-[1,1′-biphenyl]-2-yl)methyl)piperazin-1-yl)-2-(3-methoxy-3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide as a yellow solid. LC-MS: (ES, m/z): M+1=953, R,T=3.44 min. ¹H NMR (300 MHz, DMSO-d₆, ppm) δ11.00 (ds, 1H), 8.56 (s, 2H), 7.47-7.44 (m, 2H), 7.36-7.33 (m, 2H), 7.08-7.04 (m, 3H), 6.78-6..65 (m, 2H), 6.57-6.54 (m, 1H), 6.43 (s, 1H), 6.04 (s, 1H), 4.05-3.15 (m, 20H), 3.13 (s, 1H), 2.70-2.35 (m, 4H), 2.03 (s, 3H), 1.92-1.90 (m, 1H), 1.89-1.87 (m, 2H), 1.71-1.67 (m, 2H), 1.53-1.45 (m, 3H), 0.97 (s, 6H)

### Compound 3-7: Preparation of 4-(4-[[2-(4-chlorophenyl)-4,4-dimethylcyclohex-1-en-1-yl]methyl]piperazin-1-yl)-N-[4-([[(2R)-1,4-dioxan-2-yl]methyl]amino)-3-nitrobenzenesulfonyl]-2-[14-oxa-2,4,10-triazatricyclo[7.5.0.0\hat{ }[3,7]]tetradeca-1(9),2,5,7-tetraen-10-yl]benzamide

Synthesis of 4-([[(2R)-1,4-dioxan-2-yl]methyl]amino)-3-nitrobenzene-1-sulfonamide. Into a 100-mL round-bottom flask, was placed 4-fluoro-3-nitrobenzene-1-sulfonamide (1.43 g, 0.007 mmol, 1 equiv), 1-[(2R)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.8 g (87.14%) of 4-([[(2R)-1,4-dioxan-2-yl]methyl]amino)-3-nitrobenzene-1-sulfonamide as a yellow solid. LC-MS: (ES, m/z): M+1=318, R,T=0.740 min.

Synthesis of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate. Into a 100-mL round-bottom flask purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (4.35 g, 8.21 mmol, 1.87 equiv), toluene (50 mL), 4-[[2-(trimethylsilyl)ethoxy]methyl]-14-oxa-2,4,10-triazatricyclo[7.5.0.0\hat{ }[3,7]]tetradeca-1(9),2,5,7-tetraene (1.4 g, 4.39 mmol, 1 equiv), Cs₂CO₃ (7.1 g, 21.85 mmol, 4.98 equiv), XantPhos Pd 2G (584 mg, 0.66 mmol, 0.15 equiv). The resulting solution was stirred overnight at 110° C. The resulting solution was diluted with 300 mL of water. The resulting solution was extracted with 2×100 mL of ethyl acetate. The resulting mixture was washed with 1×300 mL of brine. The mixture was dried over anhydrous sodium sulfate and concentrated under vacuum. The residue was applied onto a silica gel column with ethyl acetate/petroleum ether (1:2). This resulted in 2.1 g (62%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as a brown solid. LC-MS: (ES, m/z): M+H=770, R,T=1.318 min.

Synthesis of methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate. Into a 40-mL vial, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-1 (2.1 g, 3.29 mmol, 1 equiv), THE (20 mL), TBAF.3H₂O (5 g), ethane-1,2-diamine (1.4 g). The resulting solution was stirred overnight at 70° C. in an oil bath. The resulting solution was diluted with 200 mL of water. The resulting solution was extracted with 3×30 mL of ethyl acetate. The resulting mixture was washed with 2×200 mL of brine. The mixture was dried over anhydrous sodium sulfate and concentrated under vacuum. The residue was recrystallization with ethyl acetate/petroleum ether (1:5). This resulted in 1.4 g (80%) of methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate as a white solid. LC-MS: (ES, m/z): M+H=640, R,T=1.023 min.

Synthesis of 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid. Into a 40-mL vial, was placed methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate (1.4 g, 2.24 mmol, 1 equiv), EtOH (25 mL), dioxane (25 mL), 4M NaOH (5 mL). The resulting solution was stirred for 4 h at 80° C. in an oil bath. The resulting mixture was concentrated under vacuum. The pH value of the solution was adjusted to 5-6 with HCl (2 mol/L). The solids were collected by filtration. This resulted in 1.1 g (80%) of 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid as a white solid. LC-MS: (ES, m/z): M+H=626, R,T=2.138 min.

Synthesis of 4-(4-[[2-(4-chlorophenyl)-4,4-dimethylcyclohex-1-en-1-yl]methyl]piperazin-1-yl)-N-[4-([[(2R)-1,4-dioxan-2-yl]methyl]amino)-3-nitrobenzenesulfonyl]-2-[14-oxa-2,4,10-triazatricyclo[7.5.0.0\hat{ }[3,7]]tetradeca-1(9),2,5,7-tetraen-10-yl]benzamide. Into a 40-mL vial, was placed 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid (150 mg, 0.240 mmol, 1 equiv), DCM (3 mL), 4-([[(2R)-1,4-dioxan-2-yl]methyl]amino)-3-nitrobenzene-1-sulfonamide (76 mg, 0.240 mmol, 1.00 equiv), EDCI (92 mg, 0.480 mmol, 2.00 equiv), DMAP (117 mg, 0.958 mmol, 4.00 equiv). The resulting solution was stirred overnight at 25° C. The resulting mixture was concentrated under vacuum. The crude product was purified by Prep-HPLC with the following conditions (2 #SHIMADZU (HPLC-01)): Column, XBridge Prep C18 OBD Column, 5 um,19*150 mm; mobile phase, ACN and Water (0.05% NH₃.H₂O) (20% Phase B up to 75% in 1 min, up to 95% in 7 min, hold 95% in 1 min, down to 20% in 1 min); Detector, 254/220 nm. This resulted in 28 mg (12.63%) of 4-(4-[[2-(4-chlorophenyl)-4,4-dimethylcyclohex-1-en-1-yl]methyl]piperazin-1-yl)-N-[4-([[(2R)-1,4-dioxan-2-yl]methyl]amino)-3-nitrobenzenesulfonyl]-2-[14-oxa-2,4,10-triazatricyclo[7.5.0.0\hat{ }[3,7]]tetradeca-1(9),2,5,7-tetraen-10-yl]benzamide as a yellow solid.

LC-MS: (ES, m/z): M+1=925, R,T=3.425 min. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 40° C.; flow: 1.5 mL/min. ¹H NMR (300 MHz, DMSO-d₆,ppm) δ11.90 (s, 1H), 11.20 (s, 1H), 8.60-8.41 (m, 2H), 7.64 (d, J=8.7 Hz, 1H), 7.47 (d, J=8.7 Hz, 1H), 7.36 (d, J=8.1 Hz, 2H), 7.20 (t, J=2.9 Hz, 1H), 7.07 (d, J=8.1 Hz, 2H), 6.90 (d, J=12.1 Hz, 2H), 6.70 (d, J=8.6 Hz, 2H), 6.19-6.06 (m, 1H), 4.20 (d, J=6.5 Hz, 2H), 3.88-3.71 (m, 3H), 3.69-3.32 (m, 8H), 3.28 (s, OH), 3.20 (s, 4H), 2.78 (s, 2H), 2.22 (d, J=18.1 Hz, 6H), 1.98 (s, 4H), 1.51-1.34 (m, 2H), 0.95 (s, 6H).

### Compound 3-8: Preparation of 4-(4-[[2-(4-chlorophenyl)-4,4-dimethylcyclohex-1-en-1-yl]methyl]piperazin-1-yl)-N-[4-([[(2S)-1,4-dioxan-2-yl]methyl]amino)-3-nitrobenzenesulfonyl]-2-[14-oxa-2,4,10-triazatricyclo[7.5.0.0\hat{ }[3,7]]tetradeca-1(9),2,5,7-tetraen-10-yl]benzamide

Synthesis of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate. Into a 100-mL round-bottom flask purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (4.35 g, 8.21 mmol, 1.87 equiv), toluene (50 mL), 4-[[2-(trimethylsilyl)ethoxy]methyl]-14-oxa-2,4,10-triazatricyclo[7.5.0.0\hat{ }[3,7]]tetradeca-1(9),2,5,7-tetraene (1.4 g, 4.39 mmol, 1 equiv), Cs₂CO₃ (7.1 g, 21.85 mmol, 4.98 equiv), XantPhos Pd 2G (584 mg, 0.66 mmol, 0.15 equiv). The resulting solution was stirred overnight at 110° C. The resulting solution was diluted with 300 mL of water. The resulting solution was extracted with 2×100 mL of ethyl acetate. The resulting mixture was washed with 1×300 mL of brine. The mixture was dried over anhydrous sodium sulfate and concentrated under vacuum. The residue was applied onto a silica gel column with ethyl acetate/petroleum ether (1:2). This resulted in 2.1 g (62%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as a brown solid. LC-MS: (ES, m/z): M+H=770, R,T=1.318 min.

Synthesis of methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate. Into a 40-mL vial, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-1 (2.1 g, 3.29 mmol, 1 equiv), THE (20 mL), TBAF.3H₂O (5 g), ethane-1,2-diamine (1.4 g). The resulting solution was stirred overnight at 70° C. in an oil bath. The resulting solution was diluted with 200 mL of water. The resulting solution was extracted with 3×30 mL of ethyl acetate. The resulting mixture was washed with 2×200 mL of brine. The mixture was dried over anhydrous sodium sulfate and concentrated under vacuum. The residue was recrystallization with ethyl acetate/petroleum ether (1:5). This resulted in 1.4 g (80%) of methyl 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoate as a white solid. LC-MS: (ES, m/z): M+H=640, R,T=1.023 min.

Synthesis of 4-([[(2S)-1,4-dioxan-2-yl]methyl]amino)-3-nitrobenzene-1-sulfonamide. Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as a yellow solid. LC-MS: (ES, m/z): M+1=318, R,T=0.741 min.

Synthesis of 4-(4-[[2-(4-chlorophenyl)-4,4-dimethylcyclohex-1-en-1-yl]methyl]piperazin-1-yl)-N-[4-([[(2S)-1,4-dioxan-2-yl]methyl]amino)-3-nitrobenzenesulfonyl]-2-[14-oxa-2,4,10-triazatricyclo[7.5.0.0\hat{ }[3,7]]tetradeca-1(9),2,5,7-tetraen-10-yl]benzamide. Into a 40-mL vial, was placed 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid (150 mg, 0.240 mmol, 1 equiv), DCM (3 mL), 4-([[(2S)-1,4-dioxan-2-yl]methyl]amino)-3-nitrobenzene-1-sulfonamide (76 mg, 0.240 mmol, 1.00 equiv), EDCI (92 mg, 0.480 mmol, 2.00 equiv), DMAP (117 mg, 0.958 mmol, 4.00 equiv). The resulting solution was stirred overnight at 25° C. The resulting mixture was concentrated under vacuum. The crude product was purified by Prep-HPLC with the following conditions (2 #SHIMADZU (HPLC-01)): Column, XBridge Prep C18 OBD Column, 5 um,19*150 mm; mobile phase, ACN and Water (0.05% NH₃.H₂O) (20% Phase B up to 75% in 1 min, up to 95% in 7 min, hold 95% in 1 min, down to 20% in 1 min); Detector, 254/220 nm. This resulted in 29 mg (13.08%) of 4-(4-[[2-(4-chlorophenyl)-4,4-dimethylcyclohex-1-en-1-yl]methyl]piperazin-1-yl)-N-[4-([[(2S)-1,4-dioxan-2-yl]methyl]amino)-3-nitrobenzenesulfonyl]-2-[14-oxa-2,4,10-triazatricyclo[7.5.0.0\hat{ }[3,7]]tetradeca-1(9),2,5,7-tetraen-10-yl]benzamide as a yellow solid. LC-MS: (ES, m/z): M+1=925, R,T=3.426 min. ¹H NMR (300 MHz, DMSO-d₆,ppm) δ11.90 (s, 1H), 11.20 (s, 1H), 8.60-8.41 (m, 2H), 7.64 (d, J=8.7 Hz, 1H), 7.47 (d, J=8.7 Hz, 1H), 7.36 (d, J=8.1 Hz, 2H), 7.20 (t, J=2.9 Hz, 1H), 7.07 (d, J=8.1 Hz, 2H), 6.90 (d, J=12.1 Hz, 2H), 6.70 (d, J=8.6 Hz, 2H), 6.19-6.06 (m, 1H), 4.20 (d, J=6.5 Hz, 2H), 3.88-3.71 (m, 3H), 3.69-3.32 (m, 8H), 3.28 (s, OH), 3.20 (s, 4H), 2.78 (s, 2H), 2.22 (d, J=18.1 Hz, 6H), 1.98 (s, 4H), 1.51-1.34 (m, 2H), 0.95 (s, 6H).

### Compound 3-9: Preparation of 4-(4-[[2-(4-chlorophenyl)-4, 4-dimethylcyclohex-1-en-1-yl]methyl]piperazin-1-yl)-N-[3-nitro-4-[(oxan-4-ylmethyl)amino]benzenesulfonyl]-2-[2H,3H,4H-pyrido[2,3-b][1,4]oxazepin-1-yl]benzamide

Into a 250 mL 4-necked round-bottom flask purged and maintained with an inert atmosphere of nitrogen, was placed 3, 3-diethoxypropan-1-ol (7.70 g, 64.437 mmol, 1.50 equiv), THE (60.00 mL). This was followed by the addition of NaH (3.40 g, 85.915 mmol, 2.00 equiv) in portions at 0 degrees C. To this was added 2-fluoro-3-nitropyridine (6.10 g, 42.958 mmol, 1.00 equiv) in portions at 0 degrees C. The resulting solution was stirred for 18 h at room temperature. The reaction was then quenched by the addition of 60 mL of water/ice. The resulting solution was extracted with 2×50 mL of ethyl acetate and the organic layers combined. The resulting mixture was washed with 30 ml of water. The resulting mixture was washed with 30 mL of brine. The mixture was dried over anhydrous sodium sulfate, filtered and concentrated. The residue was applied onto a silica gel column and eluted with ethyl acetate/petroleum ether (1:3). This resulted in 6.0 g(Y=51.7%) of 2-(3, 3-diethoxypropoxy)-3-methylpyridine as light yellow oil.

Into a 250-mL 3-necked round-bottom flask purged and maintained with an inert atmosphere of H₂ (2atm), was placed 1-(3, 3-diethoxypropoxy)-2-nitrobenzene (6.00 g, 22.222 mmol, 1.00 equiv), Pd/C (700.00 mg, 2.222 mmol, 0.10 equiv) and EtOH (80 ml). The resulting solution was stirred for 3h at room temperature. The solids were filtered out. The resulting mixture was concentrated. The residue was applied onto a silica gel column and eluted with ethyl acetate/petroleum ether (1:3). This resulted in 5.0 g(Y=94.3%) of 2-(3,3-diethoxypropoxy)aniline as light yellow oil.

Into a 100-mL 3-necked round-bottom flask, was placed 2-(3, 3-diethoxypropoxy) pyridin-3-amine (650.00 mg, 2.708 mmol, 1.00 equiv), DCM (10.00 mL). This was followed by the addition of TFA (1.50 g, 13.542 mmol, 5.00 equiv) dropwise with stirring at ˜10 degrees C. To this was added Triethylsilane (1.50 g, 13.542 mmol, 5.00 equiv) dropwise with stirring at ˜10 degrees C. The resulting solution was stirred for 3h at ˜10 degrees C. in an ice/salt bath. The reaction was then quenched by the addition of 10 mL of water/ice. The resulting solution was extracted with 2×20 mL of dichloromethane and the organic layers combined. The resulting mixture was washed with 20 ml of brine. The mixture was dried over anhydrous sodium sulfate and concentrated. The residue was applied onto a silica gel column and eluted with dichloromethane/methanol (10:1). This resulted in 80 mg(Y=19.7%) of 1H,2H,3H,4H-pyrido[2,3-b][1,4]oxazepine as colorless oil.

Into a 50-mL round-bottom flask purged and maintained with an inert atmosphere of nitrogen, was placed 1H,2H,3H,4H-pyrido[2,3-b][1,4]oxazepine (60.00 mg, 0.400 mmol, 1.00 equiv), methyl 2-bromo-4-(4-[[2-(4-chlorophenyl)-4,4-dimethylcyclohex-1-en-1-yl]methyl]piperazin-1-yl)benzoate (350.00 mg, 0.600 mmol, 1.50 equiv), Cs₂CO₃ (455.00 mg, 1.000 mmol, 2.50 equiv), Toluene (12 ml), 2G-Xantphos-Pd Precatalyst (11.00 mg, 0.004 mmol, 0.01 equiv). The resulting solution was stirred for 18 h at 110 degrees C. in an oil bath. The reaction mixture was cooled to room temperature with a water bath. The solids were filtered out. The resulting mixture was concentrated. The residue was applied onto a silica gel column and eluted with ethyl acetate/petroleum ether (1:3). This resulted in 125 mg(crude) of methyl 4-(4-[[2-(4-chlorophenyl)-4, 4-dimethylcyclohex-1-en-1-yl]methyl]piperazin-1-yl)-2-[2H,3H,4H-pyrido[2,3-b][1,4]oxazepin-1-yl]benzoate as colorless oil.

Into a 50-mL round-bottom flask, was placed methyl 4-(4-[[2-(4-chlorophenyl)-4, 4-dimethylcyclohex-1-en-1-yl]methyl]piperazin-1-yl)-2-[2H,3H,4H-pyrido[2,3-b][1,4]oxazepin-1-yl]benzoate (30.00 mg, 0.050 mmol, 1.00 equiv), 4 N NaOH (1.00 mL), EtOH (5.00 mL), Dioxane (5.00 mL). The resulting solution was stirred for 18h at 50 degrees C. in an oil bath. The resulting mixture was concentrated. The pH value of the solution was adjusted to 2-3 with HCl (4 mol/L). The resulting solution was extracted with 2×20 mL of ethyl acetate and the organic layers combined. The resulting mixture was washed with 20 ml of brine. The mixture was dried over anhydrous sodium sulfate and concentrated. This resulted in 20 mg(Y=68.3%) of 4-(4-[[2-(4-chlorophenyl)-4, 4-dimethylcyclohex-1-en-1-yl]methyl]piperazin-1-yl)-2-[2H,3H,4H-pyrido[2,3-b][1,4]oxazepin-1-yl]benzoic acid as colorless oil.

Into a 50-mL round-bottom flask, was placed 4-(4-[[2-(4-chlorophenyl)-4, 4-dimethylcyclohex-1-en-1-yl]methyl]piperazin-1-yl)-2-[2H,3H,4H-pyrido[2,3-b][1,4]oxazepin-1-yl]benzoic acid (20.00 mg, 0.034 mmol, 1.00 equiv), 3-nitro-4-[(oxan-4-ylmethyl) amino]benzenesulfonamide (12.00 mg, 0.038 mmol, 1.10 equiv), EDCI (13.00 mg, 0.068 mmol, 2.00 equiv), DMAP (9.00 mg, 0.068 mmol, 2.00 equiv), DCM (5.00 mL). The resulting solution was stirred for 4h at room temperature. The resulting solution was diluted with 10 mL of water. The resulting solution was extracted with 2×20 mL of dichloromethane and the organic layers combined. The resulting mixture was washed with 10 ml of brine. The mixture was dried over anhydrous sodium sulfate and concentrated. The crude product was purified by Prep-HPLC with the following conditions: column, X-Bridge Prep C18 19*150 mm 5 um; mobile phase, A: water (it contains 10 mM NH₄HCO₃ 0.05% ammonia); B: ACN; Gradient: 20-45% B in 8 min; Flow rate: 20 mL/min; detector, UV 220 nm. The collected solution was concentrated under vacuum to remove CH₃CN and the resulting solution was dried by lyophilization. This resulted in 7 mg(Y=23.2%) of 4-(4-[[2-(4-chlorophenyl)-4, 4-dimethylcyclohex-1-en-1-yl]methyl]piperazin-1-yl)-N-[3-nitro-4-[(oxan-4-ylmethyl)amino]benzenesulfonyl]-2-[2H,3H,4H-pyrido[2,3-b][1,4]oxazepin-1-yl]benzamide as a yellow solid. LC-MS-PH-PHNW-4-108-0: (ES, m/z): M+1=884, R.T=2.065 min. (DMSO, 300 ppm): 11.86 (s, 1H), 8.65 (s, 1H), 8.38 (s, 1H), 7.63-7.66 (d, J=9.0 Hz, 1H), 7.36-7.38 (m, 3H), 7.06-7.09 (m, 3H), 6.65-6.72 (m, 2H), 6.46-6.57 (m, 2H), 4.26-4.28 (m, 2H), 3.86-3.90 (m, 2H), 3.53-3.60 (m, 2H), 3.32 (s, 3H), 3.17 (s, 4H), 2.73-2.78 (m, 2H), 2.19-2.28 (m, 6H), 1.99 (s, 3H), 1.85 (s, 2H), 1.65-1.74 (m, 2H), 1.42 (s, 2H), 1.12-1.38 (m, 4H), 0.98 (s, 6H).

### Compound 3-10: Preparation of 4-(4-[[2-(4-chlorophenyl)-4,4-dimethylcyclohex-1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide

Synthesis of N-(2,2-difluoro-3-hydroxypropyl)-4-methylbenzene-1-sulfonamide: Into a 40-mL round-bottom flask, was placed 3-amino-2, 2-difluoropropan-1-ol (1 g, 9.002 mmol, 1 equiv), DCM (10 mL), Et₃N (1.37 g, 13.539 mmol, 1.50 equiv), TsCl (1.72 g, 9.002 mmol, 1.0 equiv). The resulting solution was stirred for overnight at room temperature. The resulting mixture was concentrated. The residue was applied onto a silica gel column with ethyl acetate/petroleum ether (0:1-1:2). This resulted in 600 mg (25.13%) of N-(2,2-difluoro-3-hydroxypropyl)-4-methylbenzene-1-sulfonamide as a white solid. ¹H-NMR-1(300 MHz, CDCl₃, ppm) δ7.76-7.73 (d, J=9.0 Hz, 2H), 7.35-7.32 (d, J=9.0 Hz, 2H), 5.04-5.00 (m, 1H), 3.91-3.83 (m, 2H), 3.46-3.35 (m, 2H), 2.45 (s, 3H).

Synthesis of N-[3-[(5-bromo-1-[[2-(trimethylsilyl)ethoxy]methyl]-1H-pyrrolo[2,3-b]pyridin-6-yl)oxy]-2,2-difluoropropyl]-4-met hylbenzene-1-sulfonamide: Into a 10-mL round-bottom flask, was placed 5-bromo-6-fluoro-1-[[2-(trimethylsilyl)ethoxy]methyl]-1H-pyrrolo[2,3-b]pyridine (2.60 g, 7.530 mmol, 1.00 equiv), N-(2,2-difluoro-3-hydroxypropyl)-4-methylbenzene-1-sulfonamide (2 g, 7.539 mmol, 1 equiv), dioxane (20 mL, 236.082 mmol, 31.31 equiv), This was followed by the addition of NaH (453 mg, 11.3 mmol, 1.5 equiv, 60%),in portions at 0 degrees C. The resulting solution was stirred for overnight at 90 degrees C. The resulting mixture was concentrated. The residue was applied onto a silica gel column with ethyl acetate/petroleum ether (0:1-1:5). This resulted in 2.4 g (53.90%) of N-[3-[(5-bromo-1-[[2-(trimethylsilyl)ethoxy]methyl]-1H-pyrrolo[2,3-b]pyridin-6-yl)oxy]-2,2-difluoropropyl]-4-met hylbenzene-1-sulfonamide as colorless oil. LC-MS (ES, m/z): M+1=590.

Synthesis of 12,12-difluoro-10-(4-methylben zenesulfonyl)-4-[[2-(trimethylsilyl)ethoxy]methyl]-14-oxa-2,4,10-triazatricyclo[7.5.0.0\hat{ }[3,7]]tetradeca-1,3(7),5,8-tetraene: Into a 100-mL round-bottom flask, was placed N-[3-[(5-bromo-1-[[2-(trimethylsilyl)ethoxy]methyl]-1H-pyrrolo[2,3-b]pyridin-6-yl)oxy]-2,2-difluoropropyl]-4-methylbenzene-1-sulfonamide (2.4 g, 4.064 mmol, 1 equiv), 2-(2-methylpropanoyl)cyclohexan-1-one (0.14 g, 0.832 mmol, 0.20 equiv), CuI (0.15 g, 0.813 mmol, 0.2 equiv), Cs₂CO₃ (2.65 g, 8.128 mmol, 2 equiv), DMSO (25 mL, 351.964 mmol, 86.61 equiv). The resulting solution was stirred for overnight at 120 degrees C. The resulting solution was diluted with 50 mL H₂O and 50 mL EA. The solids were filtered out. The resulting solution was extracted with 3×50 mL of ethyl acetate. The organic layer was washed with 3×50 mL of Brine. The mixture was dried over anhydrous sodium sulfate and concentrated. The residue was applied onto a silica gel column with ethyl acetate/petroleum ether (0:1-1:1). This resulted in 1.5 g (72.42%) of 12,12-difluoro-10-(4-methylben zenesulfonyl)-4-[[2-(trimethylsilyl)ethoxy]methyl]-14-oxa-2,4,10-triazatricyclo[7.5.0.0\hat{ }[3,7]]tetradeca-1,3(7),5,8-tetraene as yellow oil. ¹H NMR (300 MHz, CDCl₃, ppm): δ8.37 (s, 1H), 7.42-7.39 (m, 3H), 7.16-7.14 (d, J=6.0 Hz, 2H), 6.61-6.60 (d, J=3.0 Hz, 1H), 5.58 (s, 1H), 4.23-4.15 (m, 2H), 3.98-3.91 (m, 1H), 3.58-3.52 (m, 2H), 2.37 (s, 3H), 0.95-0.89 (m, 2H), 0.00 (s, 9H).

Synthesis of 12,12-difluoro-4-[[2-(trimethylsilyl)ethoxy]methyl]-14-oxa-2,4, 10-triazatricyclo[7.5.0.0\hat{ }[3,7]]tetradeca-1(9),2,5,7-tetraene: Into a 8-mL round-bottom flask, was placed naphthalene (2.26 g, 17.660 mmol, 6 equiv), DME (20 mL, 206.633 mmol, 70.21 equiv), Na (0.65 g, 28.255 mmol, 9.6 equiv), The resulting solution was stirred for 0.5 h at room temperature. The resulting solution was added to a 40-mL round-bottom flask, was placed 12,12-difluoro-10-(4-methylbenzenesulfonyl)-4-[[2-(trimethylsilyl)ethoxy]methyl]-14-oxa-2,4,10- triazatricyclo[7.5.0.0\hat{ }[3,7]]tetradeca-1(9),2,5,7-tetraene (1.5 g, 2.943 mmol, 1 equiv), THE (20 mL, 246.860 mmol, 83.87 equiv). The resulting solution was stirred for 1 h at room temperature. The reaction was then quenched by the addition of 10 mL of NH₄Cl. The resulting solution was extracted with 3×50 mL of ethyl acetate concentrated. The residue was applied onto a silica gel column with ethyl acetate/petroleum ether (0:1-1:1). This resulted in 0.5 g (47.79%) of 12,12-difluoro-4-[[2-(trimethylsilyl)ethoxy]methyl]-14-oxa-2,4, 10-triazatricyclo[7.5.0.0\hat{ }[3,7]]tetradeca-1(9),2,5,7-tetraene as a white solid. ¹H-NMR (300 MHz, CDCl₃, ppm): δ7.34-7.28 (m, 1H), 7.25-7.23 (d, J=6.0 Hz, 1H), 6.37-6.36 (m, 1H), 5.58-5.53 (m, 2H), 4.57-4.48 (m, 2H), 3.72-3.64 (m, 2H), 3.59-3.52 (m, 2H), 1.30-1.28 (m, 2H), 0.00 (s, 9H).

### Synthesis of methyl 4-chloro-2-(12,12-difluoro-4-[[2-(trimethylsilyl)ethoxy]methyl]-14-oxa-2,4, 10-triazatricyclo[7.5.0.0\hat{ }[3,7]]tetradeca-1(9),2,5,7-tetraen-10-yl)benzoate: Into a 8-mL round-bottom flask, was placed methyl 2-bromo-4-chlorobenzoate (210.56 mg, 0.844 mmol, 1.5equiv), 12,12-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraene (200 mg, 0.563 mmol, 1 equiv), pyridine-2-carboxylic acid (13.85 mg, 0.113 mmol, 0.2 equiv), DMSO (5 mL), Cs₂CO₃ (549.97 mg, 1.688 mmol, 3 equiv), CuI (21.43 mg, 0.113 mmol, 0.2 equiv). The resulting solution was stirred for 12 h at 130 degrees C. The reaction was then quenched by the addition of 10 mL of water. The resulting solution was extracted with 3×20 mL of ethyl acetate concentrated. The residue was applied onto a silica gel column with ethyl acetate/petroleum ether (0:1-1:10). This resulted in 180 mg (46.40%) of methyl 4-chloro-2-(12,12-difluoro-4-[[2-(trimethylsilyl)ethoxy]methyl]-14-oxa-2,4, 10-triazatricyclo[7.5.0.0\hat{ }[3,7]]tetradeca-1(9),2,5,7-tetraen-10-yl)benzoate as a white crude solid. LC-MS (ES, m/z): M+1=524

Synthesis of methyl 4-(4-[[2-(4-chlorophenyl)-4,4-dimethylcyclohex-1-en-1-yl]methyl]piperazin-1-yl)-2-(12,12-difluoro -4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 8-mL round-bottom flask, was placed methyl 4-chloro-2-(12,12-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate(180.00 mg, 0.343 mmol, 1.00 equiv), 1-[[2-(4-chlorophenyl)-4,4-dimethylcyclohex -1-en-1-yl]methyl]piperazine (131.44 mg, 0.412 mmol, 1.2 equiv), toluene (3.00 mL), Cs₂CO₃ (335.74 mg, 1.030 mmol, 3 equiv), tBuXPhos Pd G3 Precatalyst (27.29 mg, 0.034 mmol, 0.1 equiv). The resulting solution was stirred for overnight at room temperature. The resulting mixture was concentrated. The residue was applied onto a silica gel column with ethyl acetate/petroleum ether (0:1-1:1). This resulted in 100 mg of methyl 4-(4-[[2-(4-chlorophenyl)-4,4-dimethylcyclohex-1-en-1-yl]methyl]piperazin-1-yl)-2-(12,12-difluoro -4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as a white solid. LC-MS (ES, m/z): M+1=806.4

Synthesis of methyl 4-(4-[[2-(4-chlorophenyl)-4,4-dimethylcyclohex -1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]benzoate: Into a 40-mL round-bottom flask, was placed methyl 4-(4-[[2- (4-chlorophenyl)-4,4-dimethylcyclohex-1-en-1-yl]methyl]piperazin-1-yl)-2-(12,12-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (100 mg, 0.124 mmol, 1 equiv), TBAF (648.41 mg, 2.480 mmol, 20 equiv), THE (10 mL), ethane-1,2-diamine (149.04 mg, 2.480 mmol, 20 equiv). The resulting solution was stirred for 12 h at 70 degrees C. The resulting mixture was concentrated. The residue was applied onto a silica gel column with ethyl acetate/petroleum ether (0:1-1:1). This resulted in 50 mg (59.63%) of methyl 4-(4-[[2-(4-chlorophenyl)-4,4-dimethylcyclohex -1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]benzoate as a white solid. LC-MS (ES, m/z): M+1=676.3.

Synthesis of 4-(4-[[2-(4-chlorophenyl)-4,4-dimethylcyclohex-1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]benzoic acid: Into a 8-mL round-bottom flask, was placed methyl 4-(4-[[2-(4-chlorophenyl)-4,4-dimethylcyclohex-1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]benzoate (50 mg, 0.074 mmol, 1 equiv), Dioxane (1 mL), MeOH (1 mL), H₂O (1 mL), NaOH (17.74 mg, 0.444 mmol, 6.00 equiv). The resulting solution was stirred for 12 h at 70 degrees C. The pH value of the solution was adjusted to 6 with HCl (1 mol/L). The resulting mixture was concentrated. The residue was applied onto a silica gel column with ethyl acetate/petroleum ether (0:1-1:1). This resulted in 20 mg (40.85%) of 4-(4-[[2-(4-chlorophenyl)-4,4-dimethylcyclohex-1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]benzoic acid as a white solid. H-NMR (300 MHz, CDCl₃, ppm): 67.70-7.63 (m, 1H), 7.31-7.25 (m, 2H), 7.03-6.93 (m, 2H), 6.77 (s, 1H), 6.67-6.63 (m, 2H), 6.06 (s, 1H), 3.64-3.47 (m, 3H), 3.40-3.31 (m, 4H), 2.88 (s, 1H), 2.42-2.27 (m, 5H), 2.10-2.07 (m, 3H), 1.38-1.26 (m, 8H), 1.25-1.24 (m, 2H), 1.02 (s, 6H), 1.42 (m, 2H), 0.88-0.85 (m, 2H).

Synthesis of 4-(4-[[2-(4-chlorophenyl)-4,4-dimethylcyclohex-1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide: Into a 8-mL round-bottom flask, was placed 4-(4-[[2-(4-chlorophenyl)-4,4-dimethylcyclohex -1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]benzoic acid (20.00 mg, 0.030 mmol, 1.00 equiv), 3-nitro-4-[[(oxan-4-yl)methyl]amino]benzene-1-sulfonamide (11.43 mg, 0.036 mmol, 1.2 equiv), DCM (3 mL), DMAP (14.76 mg, 0.121 mmol, 4 equiv), EDCI (11.58 mg, 0.060 mmol, 2 equiv). The resulting solution was stirred for 12 h at room temperature. The crude product was purified by Prep-HPLC with the following conditions (Waters-2767): Column, X-bridge RP18, Sum, 19*100 mm; mobile phase, 0.03% ammonia in water (0.03% NH₄HCO₃ &NH₄OH) and CH₃CN (32% CH₃CN up to 52% in 6 min); Detector, UV 254 nm. This resulted in 11.5 mg (39.68%) of 4-(4-[[2-(4-chlorophenyl)-4,4-dimethylcyclohex-1-en-1-yl]methyl]piperazin-1-yl)-2-[12,12-difluoro-14-oxa-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide as a white solid. LC-MS-0: (ES, m/z): M+1=961.47. ¹H-NMR-0(300 MHz, d-DMSO, ppm): 611.01 (s, 1H), 8.34-8.33 (d, J=3.0 Hz, 1H), 7.47-7.45 (m, 2H), 7.38-7.35 (m, 2H), 6.82-6.80 (m, 3H), 6.02 (s, 1H), 3.86-3.51 (m, 7H), 3.39-3.17 (m, 5H), 2.77-2.73 (m, 2H), 2.24-2.20 (m, 6H), 2.00 (s, 2H), 1.42 (m, 2H), 1.30 (s, 1H), 0.95 (s, 6H).

### Compound 3-11: Preparation of N-(4-[[(2S)-1,4-dioxan-2-ylmethyl]amino]-3-nitrobenzenesulfonyl)-4-(4-[[2-(4-hydroxyphenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzamide

Synthesis of 4-([[(2S)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as a yellow solid. LC-MS (ES, m/z): M+1=318.

Synthesis of 2-[3-[(5-bromo-1-[[2-(trimethylsilyl)ethoxy]methyl]pyrrolo[2,3-b]pyridin-6-yl)oxy]propyl]isoindole-1,3-dione: Into a 1000-mL 3-necked round-bottom flask purged and maintained with an inert atmosphere of nitrogen, was placed 5-bromo-6-fluoro-1-[[2-(trimethylsilyl)ethoxy]methyl]pyrrolo[2,3-b]pyridine (53.50 g, 154.944 mmol, 1 equiv). This was followed by the addition of 2-(3-hydroxypropyl)isoindole-1,3-dione (31.80 g, 154.944 mmol, 1 equiv), in portions at degrees C.. To this was added Dioxane (500.00 mL) at degrees C., NaH (9.30 g, 232.415 mmol, 1.50 equiv, 60%). The resulting solution was stirred for 4 h at 80 degrees C. The reaction mixture was cooled with a water/ice bath. The reaction was then quenched by the addition of 500 mL AcOH/ice/water. The resulting solution was extracted with 2×500 mL of ethyl acetate. The resulting mixture was washed with 3×500 ml of Brine. The mixture was dried over anhydrous sodium sulfate and concentrated. The residue was applied onto a silica gel column with ethyl acetate/petroleum ether (0:1-1:10). This resulted in 58 g (70.56%) of 2-[3-[(5-bromo-1-[[2-(trimethylsilyl)ethoxy]methyl]pyrrolo[2,3-b]pyridin-6-yl)oxy]propyl]isoindole-1,3-dione as colorless oil. LC-MS: (ES, m/z): M+1=554.

Synthesis of 3-[(5-bromo-1-[[2-(trimethylsilyl)ethoxy]methyl]pyrrolo[2,3-b]pyridin-6-yl)oxy]propan-1-amine: Into a 500-mL round-bottom flask, was placed 2-[3-[(5-bromo-1-[[2-(trimethylsilyl)ethoxy]methyl]pyrrolo[2,3-b]pyridin-6-yl)oxy]propyl]isoindole-1,3-dione (58.00 g, 109.332 mmol, 1.00 equiv), EtOH (300.00 mL), NH₂NH₂H₂O (68.42 g, 1093.321 mmol, 10 equiv, 80%). The resulting solution was stirred for 4 h at room temperature. The resulting mixture was concentrated. The reaction was then quenched by the addition of 200 mL of water. The resulting solution was extracted with 2×500 mL of ethyl acetate The resulting mixture was washed with 2×300 ml of Brine. The mixture was dried over anhydrous sodium sulfate and concentrated. The residue was applied onto a silica gel column with ethyl acetate/petroleum ether (0:1-1:2). This resulted in 37.5 g (85.66%) of 3-[(5-bromo-1-[[2-(trimethylsilyl)ethoxy]methyl]pyrrolo[2,3-b]pyridin-6-yl)oxy]propan-1-amine as yellow oil.

Synthesis of 4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 1000-mL round-bottom flask, was placed 3-[(5-bromo-1-[[2-(trimethylsilyl)ethoxy]methyl]pyrrolo[2,3-b]pyridin-6-yl)oxy]propan-1-amine (37.50 g, 93.658 mmol, 1.00 equiv), toluene (500.00 mL), t-BuONa (27.00 g, 280.947 mmol, 3.00 equiv), BrettPhos Pd G3 (4.25 g, 4.688 mmol, 0.05 equiv). The resulting solution was stirred for 4 h at 110 degrees C. The solids were filtered out. The resulting mixture was concentrated. The residue was applied onto a silica gel column with ethyl acetate/petroleum ether (0:1-1:1). This resulted in 16.1 g (53.81%) of 4-[[2-(trimethylsilyl)ethoxy]methyl]-14-oxa-2,4,10-triazatricyclo[7.5.0.0\hat{ }[3,7]]tetradeca-1(9),2,5,7-tetraene as a brown solid. LC-MS: (ES, m/z): M+1=320.

Synthesis of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 500-mL round-bottom flask, was placed methyl 2-bromo-4-(4-[[2-(4-chlorophenyl)-4,4-dimethylcyclohex-1-en-1-yl]methyl]piperazin-1-yl)benzoate (46.95 g, 88.268 mmol, 2 equiv), 4-[[2-(trimethylsilyl)ethoxy]methyl]-14-oxa-2,4,10-triazatricyclo[7.5.0.0\hat{ }[3,7]]tetradeca-1(9),2,5,7-tetraene (14.10 g, 44.134 mmol, 1.00 equiv), Cs₂CO₃ (43.14 g, 132.403 mmol, 3 equiv), toluene (300 mL), xantphos Pd (2136.10 mg, 2.207 mmol, 0.05 equiv). The resulting solution was stirred for 6 h at 100 degrees C. The resulting mixture was concentrated. The residue was applied onto a silica gel column with ethyl acetate/petroleum ether (0:1-1:1). This resulted in 35 g (102.93%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as a brown crude solid. LC-MS: (ES, m/z): M+1=770.

Synthesis of 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid: Into a 1000-mL round-bottom flask, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo [7.5.0.0\hat{ }[3,7]]tetradeca-1(9),2,5,7-tetraen-10-yl)benzoate (35.00 g, 34.523 mmol, 1.00 equiv, 76%), Dioxane (200 mL), MeOH (200 mL), H₂O (100 mL), NaOH (11.05 g, 276.270 mmol, 8.00 equiv). The resulting solution was stirred for overnight at 70 degrees C. The resulting mixture was concentrated. The reaction was then quenched by the addition of 100 mL of water. The pH value of the solution was adjusted to 6 with AcOH. The solids were collected by filtration. This resulted in 31 g (118.70%) of 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid as a white crude solid. LC-MS (ES, m/z): M+1=756.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide: Into a 250-mL Into a 2000-mL round-bottom flask, was placed 4-[[(2S)-1,4-dioxan-2-ylmethyl]amino]-3-nitrobenzenesulfonamide (13.00 g, 40.980 mmol, 1 equiv), 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (31.00 g, 40.980 mmol, 1.00 equiv), DCM (1200.00 mL), DMAP (20.03 g, 163.921 mmol, 4 equiv), EDCI (15.71 g, 81.961 mmol, 2 equiv). The resulting solution was stirred for 5 days at 30 degrees C. The reaction was then quenched by the addition of 500 mL of water. The resulting solution was extracted with 2×300 mL of dichloromethane The resulting mixture was washed with 3×500 ml of Brine. The mixture was dried over anhydrous sodium sulfate and concentrated. The residue was applied onto a silica gel column with ethyl acetate/petroleum ether (0:1-1:1). This resulted in 17 g (39.29%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide as a yellow solid. LC-MS: (ES, m/z): M+1=1056.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }3,7]]tetradeca-1(9),2,5,7-tetraen-10-yl]benzamide and N-(4-[[(2S)-1,4-dioxan-2-ylmethyl]amino]-3-nitrobenzenesulfonyl)-4-(4-[[2-(4-hydroxyphenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzamide: Into a 250-mL round-bottom flask, was placed 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide (17.00 g, 16.102 mmol, 1.00 equiv), THE (100 mL), TBAF (84.20 g, 322.035 mmol, 20.00 equiv), ethane-1,2-diamine (19.35 g, 322.043 mmol, 20 equiv). The resulting solution was stirred for 3 days at 70 degrees C. The reaction was then quenched by the addition of 200 mL of water. The resulting solution was extracted with 3×100 mL of ethyl acetate. The resulting mixture was washed with 3×100 ml of Brine. The mixture was dried over anhydrous sodium sulfate and concentrated. The residue was applied onto a silica gel column with ethyl acetate/petroleum ether (0:1-1:0). This resulted in 200 mg (1.37%) of N-(4-[[(2S)-1,4-dioxan-2-ylmethyl]amino]-3-nitrobenzenesulfonyl)-4-(4-[[2-(4-hydroxyphenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzamide as a white solid. ¹H NMR-200 (300 MHz, DMSO-d₆) δ 12.29 (s, 1H), 12.05 (s, 1H), 11.34 (s, 1H), 8.00 (s, 1H), 7.52-7.42 (m, 3H), 7.37 (d, J=8.4 Hz, 2H), 7.25 (d, J=3.0 Hz, 1H), 7.07 (d, J=8.3 Hz, 2H), 6.96 (s, 1H), 6.72-6.70 (m, 2H), 6.15 (d, J=3.4, 1.9 Hz, 1H), 4.98-4.94 (m, 1H), 4.18 (s, 2H), 3.96-3.94 (m, 2H), 3.91-3.76 (m, 3H), 3.69-3.65 (m, 1H), 3.47 (s, 2H), 3.21 (s, 4H), 2.77-2.73 (m, 2H), 2.24-2.19 (m, 6H), 1.98 (s, 4H), 1.43-1.39 (m, 2H), 0.95 (s, 6H).

### Compound 3-12: Preparation of 2-[7-amino-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide

Synthesis of 5-bromo-6-fluoropyridin-2-amine: Into a 1000-mL round-bottom flask, was placed 6-fluoropyridin-2-amine (30 g, 267.601 mmol, 1 equiv), CH₃CN (500 mL), NBS (52 g, 292.161 mmol, 1.09 equiv). The resulting solution was stirred for 6 hr at 25 degrees C. The resulting solution was diluted with 1000 mL of water. The resulting solution was extracted with 3×500 mL of ethyl acetate and the organic layers combined. The resulting mixture was washed with 2×2000 ml of brine. The mixture was dried over anhydrous sodium sulfate and concentrated under vacuum. The crude product was re-crystallized from PE:EA in the ratio of 5:1. The solids were collected by filtration. This resulted in 33 g (64.56%) of 5-bromo-6-fluoropyridin-2-amine as a white solid. ¹H NMR (300 MHz, Chloroform-d, ppm) δ7.61 (t, J=8.6 Hz, 1H), 6.28 (dd, J=8.3, 1.4 Hz, 1H), 4.45 (s, 2H).

Synthesis of tert-butyl N-(5-bromo-6-fluoropyridin-2-yl)carbamate: Into a 500-mL 3-necked round-bottom flask purged and maintained with an inert atmosphere of nitrogen, was placed 5-bromo-6-fluoropyridin-2-amine (10.00 g, 52.355 mmol, 1.00 equiv), THE (100.00 mL). This was followed by the addition of NaHMDS (42.00 mL, 84.000 mmol, 1.60 equiv) dropwise with stirring at 0 degrees C. The resulting solution was stirred for 0.5 h at 0 degrees C. To this was added Boc₂O (11.47 g, 52.555 mmol, 1.00 equiv) at 0 degrees C. The resulting solution was stirred overnight at 25 degrees C. The reaction was then quenched by the addition of aqueous NH₄Cl. The resulting solution was extracted with 3×300 mL of ethyl acetate and the organic layers combined. The resulting mixture was washed with 2×1000 ml of brine. The mixture was dried over anhydrous sodium sulfate. The solids were filtered out. The resulting mixture was concentrated under vacuum. The residue was applied onto a silica gel column with ethyl acetate/petroleum ether (1:3). This resulted in 8.7 g (57.08%) of tert-butyl N-(5-bromo-6-fluoropyridin-2-yl)carbamate as a white solid. ¹H NMR (300 MHz, DMSO-d₆, ppm) δ10.23 (s, 1H), 8.17-8.11 (t, J=8.7 Hz, 1H), 7.67 (dd, J=8.6, 1.5 Hz, 1H), 1.47 (s, 9H).

Synthesis of tert-butyl N-[5-bromo-6-[3-(1,3-dioxoisoindol-2-yl)propoxy]pyridin-2-yl]carbamate: Into a 250-mL round-bottom flask purged and maintained with an inert atmosphere of nitrogen, was placed 2-(3-hydroxypropyl)isoindole-1,3-dione (6.53 g, 31.821 mmol, 1.10 equiv), THE (100.00 mL). This was followed by the addition of NaH (1.74 g, 43.504 mmol, 1.51 equiv, 60%), in portions at 0 degrees C. The resulting solution was stirred for 0.5 h at 0 degrees C. To this was added tert-butyl N-(5-bromo-6-fluoropyridin-2-yl)carbamate (8.40 g, 28.854 mmol, 1.00 equiv) at 0 degrees C. The resulting solution was stirred overnight at 70 degrees C. in an oil bath. The reaction mixture was cooled to room temperature. The reaction was then quenched by the addition of aqueous NH₄Cl. The resulting solution was extracted with 3×200 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:4). This resulted in 10.9 g (79.31%) of tert-butyl N-[5-bromo-6-[3-(1,3-dioxoisoindol-2-yl)propoxy]pyridin-2-yl]carbamate as a white solid. ¹H-NMR (300 MHz, DMSO-d₆, ppm) δ9.72 (s, 1H), 7.94-7.71 (m, 5H), 7.27 (d, J=8.4 Hz, 1H), 4.33 (t, J=5.9 Hz, 2H), 3.76 (t, J=6.7 Hz, 2H), 2.07 (p, J=6.4 Hz, 2H), 1.46 (s, 9H).

Synthesis of tert-butyl N-[6-(3-aminopropoxy)-5-bromopyridin-2-yl]carbamate: Into a 250-mL round-bottom flask, was placed tert-butyl N-[5-bromo-6-[3-(1,3-dioxoisoindol-2-yl)propoxy]pyridin-2-yl]carbamate (5.00 g, 10.497 mmol, 1.00 equiv), EtOH (50.00 mL), N₂H₄.H₂O (6.60 g, 105.600 mmol, 10.06 equiv, 80%). The resulting solution was stirred overnight at 50 degrees C. in an oil bath. The reaction mixture was cooled to room temperature. The resulting solution was diluted with 500 mL of water. The resulting solution was extracted with 3×200 mL of ethyl acetate and the organic layers combined. The resulting mixture was washed with 1×1000 ml of brine. The mixture was dried over anhydrous sodium sulfate and concentrated under vacuum. The residue was applied onto a silica gel column with dichloromethane/methanol (10:1). This resulted in 3.4 g (93.55%) of tert-butyl N-[6-(3-aminopropoxy)-5-bromopyridin-2-yl]carbamate as a yellow solid. ¹H NMR (300 MHz, DMSO-d₆, ppm) δ7.87 (d, J=8.4 Hz, 1H), 7.28 (d, J=8.4 Hz, 1H), 4.35 (t, J=6.4 Hz, 2H), 2.67 (t, J=6.7 Hz, 2H), 1.77 (p, J=6.5 Hz, 2H), 1.47 (s, 9H).

Synthesis of tert-butyl N-[1H,2H,3H,4H-pyrido[2,3-b][1,4]oxazepin-7-yl]carbamate: Into a 100-mL round-bottom flask purged and maintained with an inert atmosphere of nitrogen, was placed tert-butyl N-[6-(3-aminopropoxy)-5-bromopyridin-2-yl]carbamate (3.40 g, 9.820 mmol, 1.00 equiv), toluene (40.00 mL), t-BuONa (2.84 g, 29.552 mmol, 3.01 equiv), Xphos Pd G3 (702.00 mg, 0.829 mmol, 0.08 equiv). The resulting solution was stirred for 2 hr at 90 degrees C. in an oil bath. The reaction mixture was cooled to room temperature. The resulting mixture was concentrated under vacuum. The residue was applied onto a silica gel column with ethyl acetate/petroleum ether (1:1). This resulted in 1.11 g (42.60%) of tert-butyl N-[1H,2H,3H,4H-pyrido[2,3-b][1,4]oxazepin-7-yl]carbamate as a yellow solid. ¹H-NMR (300 MHz, DMSO-d₆, ppm) δ9.19 (s, 1H), 7.24 (d, J=8.3 Hz, 1H), 7.16 (d, J=8.3 Hz, 1H), 5.26 (t, J=3.2 Hz, 1H), 4.13-4.02 (m, 2H), 3.06 (td, J=5.8, 3.1 Hz, 2H), 1.87 (p, J=5.6 Hz, 2H), 1.44 (s, 9H).

Synthesis of methyl 2-[7-[(tert-butoxycarbonyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoate: Into a 40-mL vial purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (956.33 mg, 1.798 mmol, 1.80 equiv), toluene (10.00 mL), tert-butyl N-[1H,2H,3H,4H-pyrido[2,3-b][1,4]oxazepin-7-yl]carbamate (265.00 mg, 0.999 mmol, 1.00 equiv), Pd₂(dba)₃.CHCl₃ (206.78 mg, 0.200 mmol, 0.20 equiv), Xantphos (231.17 mg, 0.400 mmol, 0.40 equiv), Cs₂CO₃ (976.31 mg, 2.996 mmol, 3.00 equiv). The resulting solution was stirred for 2 hr at 100 degrees C. in an oil bath. The reaction mixture was cooled to room temperature. The resulting solution was diluted with 200 mL of water. The resulting solution was extracted with 3×50 mL of ethyl acetate and the organic layers combined. The resulting mixture was washed with 1×300 ml of brine. The mixture was dried over anhydrous sodium sulfate and concentrated under vacuum. The residue was applied onto a silica gel column with ethyl acetate/petroleum ether (1:2). This resulted in 430 mg (60.10%) of methyl 2-[7-[(tert-butoxycarbonyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoate as a light yellow solid. LC-MS (ES, m/z): M+1=716.

Synthesis of 2-[7-[(tert-butoxycarbonyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoic acid: Into a 40-mL vial, was placed methyl 2-[7-[(tert-butoxycarbonyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoate (300.00 mg, 0.419 mmol, 1.00 equiv), MeOH (5.00 mL), dioxane (5.00 mL), NaOH (4M, 1.00 mL). The resulting solution was stirred for 4 hr at 70 degrees C. in an oil bath. The reaction mixture was cooled to room temperature. The resulting mixture was concentrated under vacuum. The pH value of the solution was adjusted to 6-7 with HCl (2 mol/L). The resulting solution was extracted with 3×50 mL of ethyl acetate and the organic layers combined. The resulting mixture was washed with 1×300 ml of brine. The mixture was dried over anhydrous sodium sulfate and concentrated under vacuum. The residue was applied onto a silica gel column with ethyl acetate/petroleum ether (2:1). This resulted in 190 mg (64.60%) of 2-[7-[(tert-butoxycarbonyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoic acid as yellow oil. LC-MS (ES, m/z): M+1=702.

Synthesis of tert-butyl N-[1-[5-(4-[[2-(4-chlorophenyl)-4,4-dimethylcyclohex-1-en-1-yl]methyl]piperazin-1-yl)-2-[(4-[[(2S)-1,4-dioxan-2-ylmethyl]amino]-3-nitrobenzenesulfonyl)carbamoyl]phenyl]-2H,3H,4H-pyrido[2,3-b][1,4]oxazepin-7-yl]carbamate: Into a 40-mL vial, was placed 2-[7-[(tert-butoxycarbonyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoic acid (180.00 mg, 0.256 mmol, 1.00 equiv), DCM (5.00 mL), 4-[[(2S)-1,4-dioxan-2-ylmethyl]amino]-3-nitrobenzenesulfonamide (81.40 mg, 0.257 mmol, 1.00 equiv), EDCI (99.00 mg, 0.516 mmol, 2.01 equiv), DMAP (125.00 mg, 1.023 mmol, 3.99 equiv). The resulting solution was stirred overnight at 30 degrees C. in an oil bath. The resulting mixture was concentrated under vacuum. The residue was applied onto a silica gel column with dichloromethane/ethyl acetate (included 5% CH₃OH) (4:1). This resulted in 120 mg (46.75%) of tert-butyl N-[1-[5-(4-[[2-(4-chlorophenyl)-4,4-dimethylcyclohex-1-en-1-yl]methyl]piperazin-1-yl)-2-[(4-[[(2S)-1,4-dioxan-2-ylmethyl]amino]-3-nitrobenzenesulfonyl)carbamoyl]phenyl]-2H,3H,4H-pyrido[2,3-b][1,4]oxazepin-7-yl]carbamate as a yellow solid. LC-MS (ES, m/z): M+1=1001.

Synthesis of 2-[7-amino-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide: Into a 8-mL vial, was placed tert-butyl N-[1-[5-(4-[[2-(4-chlorophenyl)-4,4-dimethylcyclohex-1-en-1-yl]methyl]piperazin-1-yl)-2-[(4-[[(2S)-1,4-dioxan-2-ylmethyl]amino]-3-nitrobenzenesulfonyl)carbamoyl]phenyl]-2H,3H,4H-pyrido[2,3-b][1,4]oxazepin-7-yl]carbamate (120.00 mg, 0.120 mmol, 1.00 equiv), HCl (gas) in 1,4-dioxane (3.00 mL). The resulting solution was stirred for 4 hr at 25 degrees C. The resulting mixture was concentrated under vacuum. The pH value of the solution was adjusted to 7 with NH₃.H₂O (28%). The crude product was purified by Prep-HPLC with the following conditions (Prep-HPLC-006): Column, X Bridge Shield RP18 OBD Column, 5 um, 19*150 mm; mobile phase, Water (0.05% NH₃.H₂O) and CH₃CN (40% Phase B up to 70% in 7 min); Detector, UV 254/220 nm. This resulted in 4.1 mg (3.80%) of 2-[7-amino-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)benzamide as a yellow solid. LC-MS(ES, m/z): M+1=901, ¹H-NMR (300 MHz, DMSO-d₆, ppm) δ11.85 (s, 1H), 8.73-8.47 (m, 2H), 7.81 (dd, J=9.2, 2.3 Hz, 1H), 7.46-7.30 (m, 3H), 7.17 (d, J=9.3 Hz, 1H), 7.08 (d, J=8.4 Hz, 2H), 6.69-6.42 (m, 3H), 5.91 (d, J=8.4 Hz, 1H), 5.66 (s, 2H), 4.10 (s, 2H), 3.88-3.73 (m, 3H), 3.69-3.60 (m, 2H), 3.59-3.33 (m, 6H), 3.15 (s, 4H), 2.75 (d, J=13.5 Hz, 2H), 2.22 (d, J=16.2 Hz, 6H), 1.99 (s, 2H), 1.88 (s, 2H), 1.42 (t, J=6.4 Hz, 2H), 0.95 (s, 6H).

### Compound 3-13: Preparation of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[13-(pyridin-2-yl)-14-oxa-2,4,10-triazatricyclo[7.5.0.0\hat{ }[3,7]]tetradeca-1(9),2,5,7-tetraen-10-yl]benzamide hydrochloride

Synthesis of 3-hydroxy-3-(pyridin-2-yl)propanenitrile: Into a 100-mL round-bottom flask, was placed 3-oxo-3-(pyridin-2-yl)propanenitrile (4.00 g, 27.369 mmol, 1.00 equiv), MeOH (40.00 mL). This was followed by the addition of NaBH₄ (2.07 g, 54.739 mmol, 2.00 equiv), in portions at 0 degrees C. The resulting solution was stirred for 3 hr at room temperature. The reaction was cooled to 0 degrees C. and quenched by the addition of 5 mL of water. The resulting mixture was concentrated. The residue was applied onto a silica gel column and eluted with ethyl acetate/petroleum ether (1:3). This resulted in 2.3 g (56.72%) of 3-hydroxy-3-(pyridin-2-yl)propanenitrile as a white solid. LC-MS-1: (ES, m/z): 149.1 [M+H]⁺.

Synthesis of 3-amino-1-(pyridin-2-yl)propan-1-ol: Into a 100-mL round-bottom flask, was placed 3-hydroxy-3-(pyridin-2-yl)propanenitrile (2.30 g, 15.523 mmol, 1.00 equiv), THF (30.00 mL). This was followed by the addition of LiAlH₄ (1.18 g, 31.046 mmol, 2.00 equiv), in portions at 0 degrees C. The resulting solution was stirred for 2 hr at 0 degrees C. in a water/ice bath. The reaction was then quenched by the addition of 3 mL of water. The resulting mixture was concentrated. This resulted in 2.1 g (crude) of 3-amino-1-(pyridin-2-yl)propan-1-ol as a brown solid. LC-MS-2: (ES, m/z): 148.2 [M+H]⁺.

Synthesis of tert-butyl N-[3-hydroxy-3-(pyridin-2-yl)propyl]carbamate: Into a 100-mL round-bottom flask, was placed 3-amino-1-(pyridin-2-yl)propan-1-ol (2.00 g, crude), DCM (30 mL), Boc₂O (5.74 g, 26.282 mmol, 2.00 equiv), TEA (2.66 g, 26.287 mmol, 2.00 equiv). The resulting solution was stirred for 14 hr at room temperature. The resulting mixture was concentrated. The residue was applied onto a silica gel column and eluted with ethyl acetate/petroleum ether (1:2). This resulted in 700 mg (21.11%) of tert-butyl N-[3-hydroxy-3-(pyridin-2-yl)propyl]carbamate as an off-white solid. LC-MS-2: (ES, m/z): 253.1 [M+H]⁺.

Synthesis of ert-butyl N-[3-[(5-bromo-1-[[2-(trimethylsilyl)ethoxy]methyl]pyrrolo[2,3-b]pyridin-6-yl) oxy]-3-(pyridin-2-yl)propyl]carbamate as brown oil: Into a 50-mL round-bottom flask, was placed tert-butyl N-[3-hydroxy-3-(pyridin-2-yl)propyl]carbamate (700.00 mg, 2.774 mmol, 1.00 equiv), dioxane (15.00 mL), NaH (222.00 mg, 9.251 mmol, 3.33 equiv), 5-bromo-6-fluoro-1-[[2-(trimethylsilyl)ethoxy]methyl]pyrrolo[2,3-b]pyridine (957.94 mg, 2.774 mmol, 1.00 equiv). The resulting solution was stirred for 3 hr at 70 degrees C. The reaction was cooled to room temperature then quenched by the addition of 2 mL of water. The resulting mixture was concentrated. The residue was applied onto a silica gel column and eluted with ethyl acetate/petroleum ether (1:3). This resulted in 600 mg (37.44%) of tert-butyl N-[3-[(5-bromo-1-[[2-(trimethylsilyl)ethoxy]methyl]pyrrolo[2,3-b]pyridin-6-yl)oxy]-3-(pyridin-2-yl)propyl]carbamate as brown oil. LC-MS-4 (ES, m/z):577.2 [M+H]⁺.

Synthesis of 3-[(5-bromo-1-[[2-(trimethylsilyl)ethoxy]methyl]pyrrolo[2,3-b]pyridin-6-yl)oxy]-3- (pyridin-2-yl)propan-1-amine: Into a 50-mL round-bottom flask, was placed tert-butyl N-[3-[(5-bromo-1-[[2-(trimethylsilyl) ethoxy]methyl]pyrrolo[2,3-b]pyridin-6-yl)oxy]-3-(pyridin-2-yl)propyl]carbamate (600.00 mg, 1.039 mmol, 1.00 equiv), CH₃CN (5.00 mL), ZnBr₂ (2.34 g, 10.388 mmol, 10.00 equiv). The resulting solution was stirred for 14 hr at 80 degrees C. in an oil bath. The resulting mixture was concentrated. The residue was applied onto a silica gel column and eluted with ethyl acetate/petroleum ether (1:2). This resulted in 350 mg (70.56%) of 3-[(5-bromo-1-[[2-(trimethylsilyl)ethoxy]methyl]pyrrolo[2,3-b]pyridin-6-yl)oxy]-3-(pyridin-2-yl)propan -1-amine as yellow oil. LC-MS-4: (ES, m/z):477.2 [M+H]⁺.

Synthesis of 13-(pyridin-2-yl)-4-[[2-(trimethylsilyl)ethoxy]methyl]-14-oxa-2,4,10-triazatricyclo [7.5.0.0\hat{ }[3,7]]tetradeca-1(9),2,5,7-tetraene: Into a 50-mL round-bottom flask purged and maintained with an inert atmosphere of nitrogen, was placed 3-[(5-bromo-1-[[2-(trimethylsilyl)ethoxy]methyl]pyrrolo[2,3-b]pyridin-6-yl)oxy]-3-(pyridin-2-yl)propan-1-amine (300.00 mg, 0.628 mmol, 1.00 equiv), toluene (5.00 mL, 0.054 mmol, 0.09 equiv), BrettPhos Pd G3 (56.96 mg, 0.063 mmol, 0.10 equiv), t-BuONa (181.15 mg, 1.885 mmol, 3.00 equiv). The resulting solution was stirred for 6 hr at 80 degrees C. in an oil bath. The resulting mixture was concentrated. The residue was applied onto a silica gel column and eluted with ethyl acetate/petroleum ether (1:3). This resulted in 150 mg (60.20%) of 13-(pyridin-2-yl)-4-[[2-(trimethylsilyl)ethoxy]methyl]-14-oxa-2,4,10-triazatricyclo [7.5.0.0\hat{ }[3,7]]tetradeca-1(9),2,5,7-tetraene as a brown solid. LC-MS-6: (ES, m/z):397.2 [M+H]⁺.

### Synthesis of methyl 4-(4-[[2-(4-chlorophenyl)-4,4-dimethylcyclohex-1-en-1-yl]methyl]piperazin-1-yl) -2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate as light yellow oil: Into a 8-mL vial purged and maintained with an inert atmosphere of nitrogen, was placed 13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraene (80.00 mg, 0.202 mmol, 1.00 equiv), toluene (3.00 mL, 0.033 mmol, 0.16 equiv), methyl 2-bromo-4-(4-[[2-(4-chlorophenyl)-4,4-dimethylcyclohex-1-en-1-yl]methyl]piperazin-1-yl)benzoate (128.77 mg, 0.242 mmol, 1.20 equiv), Cs₂CO₃ (197.18 mg, 0.605 mmol, 3.00 equiv), xanphos Pd 2G (17.89 mg, 0.020 mmol, 0.10 equiv). The resulting solution was stirred for 3 hr at 110 degrees C. in an oil bath. The resulting mixture was concentrated. The residue was applied onto a silica gel column and eluted with ethyl acetate/petroleum ether (1:1). This resulted in 75 mg (43.86%) of methyl 4-(4-[[2-(4-chlorophenyl)-4,4-dimethylcyclohex-1-en-1-yl]methyl]piperazin-1-yl)-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate as light yellow oil. LC-MS-7 (ES, m/z):847.5 [M+H]⁺

Synthesis of 4-(4-[[2-(4-chlorophenyl)-4,4-dimethylcyclohex-1-en-1-yl]methyl]piperazin-1-yl)-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid: Into a 8-mL round-bottom flask, was placed methyl 4-(4-[[2-(4-chlorophenyl)-4,4-dimethylcyclohex -1-en-1-yl]methyl]piperazin-1-yl)-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate (80.00 mg, 0.094 mmol, 1.00 equiv), MeOH (0.50 mL), Dioxane (0.50 mL), H₂O (0.25 mL, 0.014 mmol, 0.15 equiv), NaOH (15.10 mg, 0.378 mmol, 4.00 equiv). The resulting solution was stirred for 14 hr at 70 degrees C. in an oil bath. The resulting mixture was concentrated. The resulting solution was diluted with 2 mL of H₂O. The pH value of the solution was adjusted to 6 with AcOH. The resulting solution was extracted with 3×2 mL of dichloromethane concentrated. The residue was applied onto a silica gel column and eluted with dichloromethane/methanol (10:1). This resulted in 60 mg (76.26%) of 4-(4-[[2-(4-chlorophenyl)-4,4-dimethylcyclohex-1-en-1-yl]methyl]piperazin-1-yl)-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid as a white solid. LC-MS-8 (ES, m/z):833.4 [M+H]⁺.

### Synthesis of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide: Into a 8-mL vial, was placed 4-(4-[[2-(4-chlorophenyl)-4,4-dimethylcyclohex-1-en-1-yl]methyl]piperazin-1-yl)-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid (20.00 mg, 0.024 mmol, 1.00 equiv), 3-nitro-4-[(oxan-4-ylmethyl)amino]benzenesulfonamide (7.57 mg, 0.024 mmol, 1.00 equiv), DCM (1.00 mL), EDCI (9.20 mg, 0.048 mmol, 2.00 equiv), DMAP (5.86 mg, 0.048 mmol, 2.00 equiv). The resulting solution was stirred for 14 hr at room temperature. The resulting mixture was concentrated. The residue was purified by Prep-TLC with dichloromethane/methanol (10:1). This resulted in 17 mg (62.65%) of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide as a yellow solid. LC-MS-8 (ES, m/z):1130.7 [M+H]⁺

Synthesis of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[13-(pyridin-2-yl)-14-oxa-2,4,10-triazatricyclo[7.5.0.0\hat{ }[3,7]]tetradeca-1(9),2,5,7-tetraen-10-yl]benzamide hydrochloride: Into a 8-mL round-bottom flask, was placed 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[13-(pyridin-2-yl)-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide (15.00 mg), TBAF in THF(2M, 1.00 mL), ethane-1,2-diamine (0.25 mL). The resulting solution was stirred for 14 hr at 70 degrees C. in an oil bath. The resulting mixture was concentrated. The residue was purified by Prep-TLC with dichloromethane/methanol (10:1). The crude product was purified by Prep-HPLC with the following conditions: Column, XBridge Prep C18 OBD 19*150 mm 5 um; mobile phase, A: 0.1% HCl in water; B: ACN; Gradient: 6-95% B in 7.9 min; Flow rate: 20 ml/min; Detector, 220 nm. This resulted in 3.2 mg (23.8%) of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[13-(pyridin-2-yl)-14-oxa-2,4,10-triazatricyclo[7.5.0.0\hat{ }[3,7]]tetradeca-1(9),2,5,7-tetraen-10-yl]benzamide hydrochloride as a yellow solid. LC-MS-0 (ES, m/z): 1000.5 [M-HCl+H],H NMR-0 (300 MHz, CDCl₃, ppm): δ8.81 (s, 1H), 8.62-8.57 (m, 1H), 8.42-8.25 (m, 1H), 8.01-8.75 (m, 3H), 7.45-7.34 (m, 3H), 7.18-7.10 (m, 1H), 7.05-6.88 (m, 3H), 6.83-6.55 (m, 3H), 6.27-6.17 (m, 1H), 5.19 (s, 1H), 4.13-3.90 (m, 3H), 3.82-3.33 (m, 10H), 3.24-3.02 (m, 2H), 2.70-2.48 (m, 2H), 2.43-2.31 (m, 2H), 2.17-2.08 (m, 2H), 1.96-1.8 (m, 1H), 1.70-1.63 (m, 2H), 1.58-1.52 (m, 2H), 1.46-1.20 (m, 6H), 1.01 (d, J=2.1 Hz, 6H).

### Compound 3-14: Preparation of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[13-oxa-2,4,10-triazatricyclo[7.5.0.0\hat{ }[3,7]]tetradeca-1(9),2,5,7-tetraen-10-yl]benzamide hydrochloride

Synthesis of methyl 6-amino-3-bromo-5-iodopyridine-2-carboxylate: Into a 500-mL round-bottom flask, was placed methyl 6-amino-3-bromopyridine-2-carboxylate (20.00 g, 86.562 mmol, 1.00 equiv), HOAc (200.00 mL), TFA (10.00 mL). This was followed by the addition of NIS (29.35 g, 130.453 mmol, 1.51 equiv), in portions at 25 degrees C. The resulting solution was stirred overnight at 25 degrees C. The resulting solution was diluted with 2000 mL of water. The resulting solution was extracted with 3×500 mL of ethyl acetate and the organic layers combined. The resulting mixture was washed with 2×2000 mL of brine. The mixture was dried over anhydrous sodium sulfate and concentrated under vacuum. The crude product was re-crystallized from PE:EA in the ratio of 1:1. The solids were collected by filtration. This resulted in 25.8 g (83.50%) of methyl 6-amino-3-bromo-5-iodopyridine-2-carboxylate as a red solid. ¹H-NMR (300 MHz, Chloroform-d, ppm) δ8.14 (s, 1H), 5.21 (br, 2H), 3.99 (s, 3H).

Synthesis of methyl 6-amino-3-bromo-5-(2-ethoxyethenyl)pyridine-2-carboxylate: Into a 500-mL round-bottom flask purged and maintained with an inert atmosphere of nitrogen, was placed methyl 6-amino-3-bromo-5-iodopyridine-2-carboxylate (24.00 g, 67.237 mmol, 1.00 equiv), 2-(2-ethoxyethenyl)-4,4,5,5-tetramethyl-1,3,2-dioxaborolane (26.50 g, 133.791 mmol, 1.99 equiv), i-PrOH (300 mL), K₃PO₄ (42.60 g, 200.691 mmol, 2.98 equiv), Pd(OAc)₂ (1.50 g, 6.681 mmol, 0.10 equiv), Ruphos (3.13 g, 6.708 mmol, 0.10 equiv). The resulting solution was stirred overnight at 30 degrees C. in an oil bath. The reaction mixture was cooled to room temperature. The solids were filtered out. The resulting mixture was concentrated. The residue was applied onto a silica gel column with ethyl acetate/petroleum ether (1:1). This resulted in 10.7 g (52.85%) of methyl 6-amino-3-bromo-5-(2-ethoxyethenyl)pyridine-2-carboxylate as brown oil. ¹H NMR (300 MHz, Chloroform-d, ppm) δ8.10 (s, 0.5H), 7.57 (s, 0.5H), 6.90 (d, J=12.6 Hz, 0.5H), 6.41 (d, J=7.1 Hz, 0.5H), 3.97 (s, 3H).

Synthesis of methyl 5-bromo-1H-pyrrolo[2,3-b]pyridine-6-carboxylate: Into a 250-mL round-bottom flask, was placed methyl 6-amino-3-bromo-5-(2-ethoxyethenyl)pyridine-2-carboxylate (10.70 g, 35.532 mmol, 1.00 equiv), MeOH (100.00 mL), Conc.HCl (20.00 mL, 12 N). The resulting solution was stirred overnight at 25 degrees C. The resulting mixture was concentrated under vacuum. The pH value of the solution was adjusted to 7 with NaOH (2 mol/L). The solids were collected by filtration. This resulted in 9 g (99.30%) of methyl 5-bromo-1H-pyrrolo[2,3-b]pyridine-6-carboxylate as a brown solid. ¹H NMR (300 MHz, Chloroform-d, ppm) δ11.60 (s, 1H), 8.29 (s, 1H), 7.69 (t, J=3.0 Hz, 1H), 6.54 (dd, J=3.4, 1.7 Hz, 1H), 4.10 (s, 3H).

Synthesis of methyl 5-bromo-1-[[2-(trimethylsilyl)ethoxy]methyl]pyrrolo[2,3-b]pyridine-6-carboxylate: Into a 250-mL round-bottom flask purged and maintained with an inert atmosphere of nitrogen, was placed methyl 5-bromo-1H-pyrrolo[2,3-b]pyridine-6-carboxylate (9.00 g, 35.284 mmol, 1.00 equiv), DMF (100.00 mL). This was followed by the addition of NaH (2.83 g, 70.757 mmol, 2.01 equiv, 60%), in portions at 0 degrees C. The resulting solution was stirred for 30 min at 0 degrees C. To this was added SEM-Cl (8.82 g, 52.903 mmol, 1.50 equiv) at 0 degrees C. The resulting solution was stirred for 2 hr at 25 degrees C. The reaction was then quenched by the addition of 500 mL of water. The resulting solution was extracted with 2×200 mL of ethyl acetate and the organic layers combined. The resulting mixture was washed with 1×500 mL of brine. The mixture was dried over anhydrous sodium sulfate and concentrated under vacuum. The residue was applied onto a silica gel column with ethyl acetate/petroleum ether (1:3). This resulted in 8.2 g (60.31%) of methyl 5-bromo-1-[[2-(trimethylsilyl)ethoxy]methyl]pyrrolo[2,3-b]pyridine-6-carboxylate as yellow oil. ¹H NMR (300 MHz, Chloroform-d, ppm) δ8.17 (s, 1H), 7.50 (d, J=3.7 Hz, 1H), 6.53 (d, J=3.6 Hz, 1H), 5.68 (s, 2H), 4.02 (s, 3H), 3.60-3.45 (m, 2H), 0.99-0.88 (m, 2H),-0.05 (s, 9H).

Synthesis of (5-bromo-1-[[2-(trimethylsilyl)ethoxy]methyl]pyrrolo[2,3-b]pyridin-6-yl)methanol: Into a 250-mL round-bottom flask, was placed methyl 5-bromo-1-[[2-(trimethylsilyl)ethoxy]methyl]pyrrolo[2,3-b]pyridine-6-carboxylate (5.00 g, 12.976 mmol, 1.00 equiv), THE (50.00 mL). This was followed by the addition of LAH (990.00 mg, 26.084 mmol, 2.01 equiv), in portions at 0 degrees C. The resulting solution was stirred for 2 hr at 25 degrees C. The reaction was then quenched by the addition of Na₂SO₄.10H₂O. The solids were filtered out. The resulting mixture was concentrated under vacuum. The residue was applied onto a silica gel column with ethyl acetate/petroleum ether (1:3). This resulted in 1.7 g (36.67%) of (5-bromo-1-[[2-(trimethylsilyl)ethoxy]methyl]pyrrolo[2,3-b]pyridin-6-yl)methanol as light yellow oil. ¹H NMR (300 MHz, Chloroform-d, ppm) δ8.10 (s, 1H), 7.36 (d, J=3.6 Hz, 1H), 6.51 (d, J=3.6 Hz, 1H), 5.68 (s, 2H), 4.85 (s, 2H), 3.63-3.50 (m, 2H), 0.98-0.88 (m, 2H), -0.05 (s, 9H).

Synthesis of ethyl 2-[(5-bromo-1-[[2-(trimethylsilyl)ethoxy]methyl]pyrrolo[2,3-b]pyridin-6-yl)methoxy]acetate: Into a 100-mL round-bottom flask purged and maintained with an inert atmosphere of nitrogen, was placed (5-bromo-1-[[2-(trimethylsilyl)ethoxy]methyl]pyrrolo[2,3-b]pyridin-6-yl)methanol (1.70 g, 4.758 mmol, 1.00 equiv), THE (20.00 mL). This was followed by the addition of NaH (669.00 mg, 16.727 mmol, 3.52 equiv, 60%), in portions at 0 degrees C. The resulting solution was stirred for 30 min at 0 degrees C. To this was added ethyl bromoacetate (2.38 g, 14.251 mmol, 3.00 equiv) at 0 degrees C. The resulting solution was stirred overnight at 70 degrees C. in an oil bath. The reaction mixture was cooled to room temperature. The reaction was then quenched by the addition of 200 mL of aqueous NH₄Cl. The resulting solution was extracted with 2×100 mL of ethyl acetate and the organic layers combined. The resulting mixture was washed with 1×500 mL of brine. The mixture was dried over anhydrous sodium sulfate and concentrated under vacuum. The residue was applied onto a silica gel column with ethyl acetate/petroleum ether (1:3). This resulted in 1.7 g (80.58%) of ethyl 2-[(5-bromo-1-[[2-(trimethylsilyl)ethoxy]methyl]pyrrolo[2,3-b]pyridin-6-yl)methoxy]acetate as light yellow oil. ¹H NMR (300 MHz, Chloroform-d, ppm) δ8.10 (s, 1H), 7.39 (d, J=3.6 Hz, 1H), 6.48 (d, J=3.6 Hz, 1H), 5.68 (s, 2H), 4.99 (s, 2H), 4.28 (s, 2H), 4.23 (t, J=7.1 Hz, 2H), 3.58-3.51 (m, 2H), 1.34-1.27 (m, 3H), 0.95-0.88 (m, 2H), -0.05 (s, 9H).

Synthesis of 2-[(5-bromo-1-[[2-(trimethylsilyl)ethoxy]methyl]pyrrolo[2,3-b]pyridin-6-yl)methoxy]acetamide: Into a 50-mL pressure tank reactor, was placed ethyl 2-[(5-bromo-1-[[2-(trimethylsilyl)ethoxy]methyl]pyrrolo[2,3-b]pyridin-6-yl)methoxy]acetate (1.70 g, 3.834 mmol, 1.00 equiv), NH₃(g) in MeOH (20.00 mL). The resulting solution was stirred overnight at 60 degrees C. in an oil bath. The reaction mixture was cooled to room temperature. The resulting mixture was concentrated under vacuum. The crude product was re-crystallized from PE:EA in the ratio of 5:1. The solids were collected by filtration. This resulted in 1.2 g (75.53%) of 2-[(5-bromo-1-[[2-(trimethylsilyl)ethoxy]methyl]pyrrolo[2,3-b]pyridin-6-yl)methoxy]acetamide as a white solid. ¹H NMR (300 MHz, Chloroform-d, ppm) δ8.13 (s, 1H), 7.40 (d, J=3.6 Hz, 1H), 6.50 (d, J=3.6 Hz, 1H), 5.65 (s, 2H), 4.95 (s, 2H), 4.19 (s, 2H), 3.64-3.46 (m, 2H), 1.03-0.85 (m, 2H), -0.04 (s, 9H).

### Synthesis of 4-[[2-(trimethylsilyl)ethoxy]methyl]-13-oxa-2,4,10-triazatricyclo[7.5.0.0\hat{ }[3,7]]tetradeca-1(9),2,5,7-tetraen-11-one: Into a 100-mL round-bottom flask purged and maintained with an inert atmosphere of nitrogen, was placed 2-[(5-bromo-1-[[2-(trimethylsilyl)ethoxy]methyl]pyrrolo[2,3-b]pyridin-6-yl)methoxy]acetamide (1.20 g, 2.896 mmol, 1.00 equiv), dioxane (20.00 mL), methyl[2-(methylamino)ethyl]amine (256.00 mg, 2.904 mmol, 1.00 equiv), CuI (553.00 mg, 2.904 mmol, 1.00 equiv), K₂CO₃ (1.20 g, 8.683 mmol, 3.00 equiv). The resulting solution was stirred overnight at 100 degrees C. in an oil bath. The reaction mixture was cooled to room temperature. The resulting mixture was concentrated under vacuum. The residue was applied onto a silica gel column with ethyl acetate/petroleum ether (1:3). This resulted in 527 mg (54.57%) of 4-[[2-(trimethylsilyl)ethoxy]methyl]-13-oxa-2,4,10-triazatricyclo[7.5.0.0\hat{ }[3,7]]tetradeca-1(9),2,5,7-tetraen-11-one as a white solid. ¹H NMR (300 MHz, Chloroform-d, ppm) δ8.65 (br, 1H), 7.55 (s, 1H), 7.38 (d, J=3.6 Hz, 1H), 6.49 (d, J=3.6 Hz, 1H), 5.65 (s, 2H), 5.00 (s, 2H), 4.64 (s, 2H), 3.66-3.45 (m, 2H), 0.98-0.87 (m, 2H), -0.04 (s, 9H)

Synthesis of 4-[[2-(trimethylsilyl)ethoxy]methyl]-13-oxa-2,4,10-triazatricyclo[7.5.0.0\hat{ }[3,7]]tetradeca-1(9),2,5,7-tetraene: Into a 100-mL round-bottom flask, was placed 4-[[2-(trimethylsilyl)ethoxy]methyl]-13-oxa-2,4,10-triazatricyclo[7.5.0.0\hat{ }[3,7]]tetradeca-1(9),2,5,7-tetraen-11-one (527.00 mg, 1.580 mmol, 1.00 equiv), THE (10.00 mL), LAH (150.00 mg, 3.952 mmol, 2.50 equiv). The resulting solution was stirred for 3 hr at 25 degrees C. The reaction was then quenched by the addition of Na₂SO₄.10H₂O. The solids were filtered out. The resulting mixture was concentrated under vacuum. The residue was applied onto a silica gel column with ethyl acetate/petroleum ether (1:3). This resulted in 350 mg (69.32%) of 4-[[2-(trimethylsilyl)ethoxy]methyl]-13-oxa-2,4,10-triazatricyclo[7.5.0.0\hat{ }[3,7]]tetradeca-1(9),2,5,7-tetraene as yellow oil. LC-MS (ES, m/z): M+1=320.

Synthesis of methyl 4-(4-[[2-(4-chlorophenyl)-4,4-dimethylcyclohex-1-en-1-yl]methyl]piperazin-1-yl)-2-(4-j[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzoate: Into a 40-mL vial purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (1.16 g, 2.181 mmol, 1.99 equiv), toluene (20.00 mL), 4-[[2-(trimethylsilyl)ethoxy]methyl]-13-oxa-2,4,10-triazatricyclo[7.5.0.0\hat{ }[3,7]]tetradeca-1(9),2,5,7-tetraene (350.00 mg, 1.096 mmol, 1.00 equiv), Cs₂CO₃ (1.07 g, 3.284 mmol, 3.00 equiv), Xantphos Pd 2G (146.00 mg, 0.165 mmol, 0.15 equiv). The resulting solution was stirred for 2 days at 110 degrees C. in an oil bath. The reaction mixture was cooled to room temperature. The resulting mixture was concentrated under vacuum. The residue was applied onto a silica gel column with ethyl acetate/petroleum ether (1:1). The crude product was purified by Flash-Prep-HPLC with the following conditions (IntelFlash-1): Column, C18 reversed phase column; mobile phase, water (0.01% TFA) and CH₃CN; Gradient: 20% CH₃CN increasing to 80% within 15 min; Flow rate: 80 mL/min; Detector, 254/220 nm. This resulted in 150 mg (17.77%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzoate as yellow oil. LC-MS (ES, m/z): M+1=770.

Synthesis of 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzoic acid: Into a 100-mL round-bottom flask, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzoate (150.00 mg, 0.195 mmol, 1.00 equiv), MeOH (5.00 mL), dioxane (5.00 mL), NaOH (1.00 mL, 4.000 mmol, 20.55 equiv). The resulting solution was stirred for 4 hr at 70 degrees C. in an oil bath. The reaction mixture was cooled to room temperature. The resulting mixture was concentrated under vacuum. The pH value of the solution was adjusted to 6-7 with HCl (2 mol/L). The resulting solution was extracted with 3×50 mL of ethyl acetate and the organic layers combined. The resulting mixture was washed with 1×200 mL of brine. The mixture was dried over anhydrous sodium sulfate and concentrated under vacuum. The residue was applied onto a silica gel column with ethyl acetate/petroleum ether (1:1). This resulted in 100 mg (67.90%) of 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzoic acid as light yellow oil. LC-MS: (ES, m/z): M+1=756.

### Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzamide: Into a 40-mL vial, was placed 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzoic acid (100.00 mg, 0.132 mmol, 1.00 equiv), DCM (5.00 mL), 4-[[(2S)-1,4-dioxan-2-ylmethyl]amino]-3-nitrobenzenesulfonamide (42.00 mg, 0.132 mmol, 1.00 equiv), EDCI (51.00 mg, 0.266 mmol, 2.01 equiv), DMAP (64.00 mg, 0.524 mmol, 3.96 equiv). The resulting solution was stirred overnight at 30 degrees C. in an oil bath. The resulting mixture was concentrated under vacuum. The residue was applied onto a silica gel column with dichloromethane/ethyl acetate (1:1). This resulted in 50 mg (35.83%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzamide as a yellow solid

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[13-oxa-2,4,10-triazatricyclo[7.5.0.0\hat{ }[3,7]]tetradeca-1(9),2,5,7-tetraen-10-yl]benzamide hydrochloride: Into a 40-mL vial, was placed 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-13-oxa-2,4,10-triazatricyclo[7.5.0.0\hat{ }[3,7]]tetradeca-1(9),2,5,7-tetraen-10-yl)benzamide (50.00 mg, 0.047 mmol, 1.00 equiv), 1.0 M TBAF/THF (5.00 mL), ethylenediamine (100.00 mg, 1.664 mmol, 35.13 equiv). The resulting solution was stirred for 3.5 hr at 70 degrees C. in an oil bath. The reaction mixture was cooled to room temperature. The resulting solution was diluted with 100 mL of H₂O. The resulting solution was extracted with 3×30 mL of dichloromethane and the organic layers combined. The resulting mixture was washed with 1×200 mL of brine. The mixture was dried over anhydrous sodium sulfate and concentrated under vacuum. The residue was purified by Prep-TLC with dichloromethane/ethyl acetate (included 10% MeOH) (1:1). The crude product was purified by Prep-HPLC with the following conditions: Column, SunFire Prep C18, 19×150 mm, Sum; Moile phase, water (it contains 0.1% FA) and CH₃CN; Gradient: 20% up to 85% in 15 min; Flow rate: 15 mL/min; Detector: 254/220 nm. The collected solution was concentrated under vacuum to remove CH₃CN and the resulting solution was dried by lyophilization (added with Conc.HCl (1 drop)). This resulted in 4.5 mg (9.88%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[13-oxa-2,4,10-triazatricyclo[7.5.0.0\hat{ }[3,7]]tetradeca-1(9),2,5,7-tetraen-10-yl]benzamide hydrochloride as a yellow solid. LC-MS (ES, m/z): M+1=925. ¹H NMR (300 MHz, DMSO-d₆, ppm) δ11.80 (s, 1H), 11.22 (s, 1H), 10.71 (d, J=10.0 Hz, 1H), 8.62 (t, J=5.2 Hz, 1H), 8.32 (d, J=2.0 Hz, 1H), 7.55-7.21 (m, 5H), 7.14 (d, J=7.9 Hz, 2H), 6.98-6.58 (m, 4H), 6.13 (s, 1H), 4.76 (s, 2H), 4.03-3.46 (m, 17H), 3.13 (d, J=30.4 Hz, 2H), 2.78 (q, J=11.2, 10.0 Hz, 2H), 2.40 (s, 2H), 2.05 (s, 2H), 1.46 (q, J=6.3 Hz, 2H), 1.23 (s, 2H), 0.97 (s, 6H).

### Compound 3-15: Preparation of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[7-(methylamino)-2H,3H,4H-pyrido[2,3-b][1,4]oxazepin-1-yl]benzamide hydrochloride

Synthesis of tert-butyl N-(5-bromo-6-fluoropyridin-2-yl)-N-methylcarbamate: Into a 250-mL round-bottom flask purged and maintained with an inert atmosphere of nitrogen, was placed tert-butyl N-(5-bromo-6-fluoropyridin-2-yl)carbamate (3.30 g, 11.336 mmol, 1.00 equiv), DMF (50.00 mL). This was followed by the addition of NaH (570.00 mg, 14.251 mmol, 1.26 equiv, 60%), in portions at 0 degrees C. The resulting solution was stirred for 30 min at 0 degrees C. To this was added CH₃I (1.86 g, 13.104 mmol, 1.16 equiv) at 0 degrees C. The resulting solution was stirred for 1 hr at 25 degrees C. The reaction was then quenched by the addition of 500 mL of aqueous NH₄Cl. The resulting solution was extracted with 2×200 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:3). This resulted in 3.48 g (100.61%) of tert-butyl N-(5-bromo-6-fluoropyridin-2-yl)-N-methylcarbamate as a white solid. LC-MS (ES, m/z): M-t-Bu+1=249.

Synthesis of tert-butyl N-[5-bromo-6-[3-(1,3-dioxoisoindol-2-yl)propoxy]pyridin-2-yl]-N-methylcarbamate: Into a 250-mL round-bottom flask purged and maintained with an inert atmosphere of nitrogen, was placed 2-(3-hydroxypropyl)isoindole-1,3-dione (2.58 g, 12.572 mmol, 1.10 equiv), THE (40.00 mL). This was followed by the addition of NaH (687.00 mg, 17.177 mmol, 1.50 equiv, 60%), in portions at 0 degrees C. The resulting solution was stirred for 30 min at 0 degrees C. To this was added tert-butyl N-(5-bromo-6-fluoropyridin-2-yl)-N-methylcarbamate (3.49 g, 11.437 mmol, 1.00 equiv) at 0 degrees C. The resulting solution was stirred for 2 hr at 70 degrees C. in an oil bath. The reaction mixture was cooled to room temperature. The reaction was then quenched by the addition of 500 mL of aqueous NH₄Cl. The resulting solution was extracted with 2×200 mL of ethyl acetate and the organic layers combined. The resulting mixture was washed with 1×1000 ml of brine. The mixture was dried over anhydrous sodium sulfate and concentrated under vacuum. The residue was applied onto a silica gel column with ethyl acetate/petroleum ether (1:3). This resulted in 4.2 g (74.89%) of tert-butyl N-[5-bromo-6-[3-(1,3-dioxoisoindol-2-yl)propoxy]pyridin-2-yl]-N-methylcarbamate as a white solid. ¹H NMR (300 MHz, DMSO-d₆, ppm) δ7.90-7.78 (m, 5H), 7.20 (d, J=8.4 Hz, 1H), 4.35 (t, J=6.0 Hz, 2H), 3.77 (t, J=6.6 Hz, 2H), 3.22 (s, 3H), 2.09 (p, J=6.4 Hz, 2H), 1.46 (s, 9H).

Synthesis of tert-butyl tert-butyl N-[6-(3-aminopropoxy)-5-bromopyridin-2-yl]-N-methylcarbamate: Into a 250-mL round-bottom flask, was placed tert-butyl N-[5-bromo-6-[3-(1,3-dioxoisoindol-2-yl)propoxy]pyridin-2-yl]-N-methylcarbamate (4.20 g, 8.565 mmol, 1.00 equiv), EtOH (50.00 mL), N₂H₄.H₂O (5.37 g, 85.909 mmol, 10.03 equiv, 80%). The resulting solution was stirred overnight at 50 degrees C. in an oil bath. The reaction mixture was cooled to room temperature. The resulting solution was diluted with 500 mL of water. The resulting solution was extracted with 2×200 mL of ethyl acetate and the organic layers combined. The resulting mixture was washed with 1×1000 ml of brine. The mixture was dried over anhydrous sodium sulfate and concentrated under vacuum. This resulted in 3.02 g (97.87%) of tert-butyl N-[6-(3-aminopropoxy)-5-bromopyridin-2-yl]-N-methylcarbamate as yellow oil. LC-MS (ES, m/z): M+1=360.

Synthesis of tert-butyl N-methyl-N-[1H,2H,3H,4H-pyrido[2,3-b][1,4]oxazepin-7-yl]carbamate: Into a 100-mL round-bottom flask purged and maintained with an inert atmosphere of nitrogen, was placed tert-butyl N-[6-(3-aminopropoxy)-5-bromopyridin-2-yl]-N-methylcarbamate (3.00 g, 8.328 mmol, 1.00 equiv), toluene (30.00 mL), Xphos Pd G3 (600.00 mg, 0.709 mmol, 0.09 equiv), t-BuONa (2.41 g, 25.077 mmol, 3.01 equiv). The resulting solution was stirred for 2 hr at 90 degrees C. in an oil bath. The reaction mixture was cooled to room temperature. The resulting mixture was concentrated under vacuum. The residue was applied onto a silica gel column with ethyl acetate/petroleum ether (1:2). This resulted in 1 g (42.99%) of tert-butyl N-methyl-N-[1H,2H,3H,4H-pyrido[2,3-b][1,4]oxazepin-7-yl]carbamate as a brown solid. ¹H NMR (300 MHz, DMSO-d₆, ppm) δ7.16 (d, J=8.2 Hz, 1H), 6.97 (d, J=8.2 Hz, 1H), 5.47 (t, J=3.2 Hz, 1H), 4.11 (dd, J=6.1, 4.8 Hz, 2H), 3.18-3.08 (m, 2H), 3.12 (s, 3H), 1.89 (p, J=5.7 Hz, 2H), 1.42 (s, 9H).

Synthesis of methyl 2-[7-[(tert-butoxycarbonyl)(methyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoate: Into a 40-mL vial purged and maintained with an inert atmosphere of nitrogen, was placed tert-butyl N-methyl-N-[1H,2H,3H,4H-pyrido[2,3-b][1,4]oxazepin-7-yl]carbamate (500.00 mg, 1.790 mmol, 1.00 equiv), toluene (20.00 mL), methyl 2-bromo-4-(4-[[2-(4-chlorophenyl)-4,4-dimethylcyclohex-1-en-1-yl]methyl]piperazin-1-yl)benzoate (1.71 g, 3.215 mmol, 1.80 equiv), Pd₂(dba)₃.CHCl₃ (369.00 mg, 0.356 mmol, 0.20 equiv), Xantphos (414.00 mg, 0.715 mmol, 0.40 equiv), Cs₂CO₃ (1.75 g, 5.371 mmol, 3.00 equiv). The resulting solution was stirred for 2 hr at 100 degrees C. in an oil bath. The reaction mixture was cooled to room temperature. The resulting mixture was concentrated under vacuum. The residue was applied onto a silica gel column with ethyl acetate/petroleum ether (1:2). This resulted in 1.1 g (84.14%) of methyl 2-[7-[(tert-butoxycarbonyl)(methyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoate as a yellow solid. LC-MS (ES, m/z): M+1=730.

Synthesis of 2-[7-[(tert-butoxycarbonyl)(methyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoic acid: Into a 100-mL round-bottom flask, was placed methyl 2-[7-[(tert-butoxycarbonyl)(methyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoate (700.00 mg, 0.958 mmol, 1.00 equiv), dioxane (5.00 mL), MeOH (5.00 mL), NaOH (1.00 mL, 4.000 mmol, 4.17 equiv). The resulting solution was stirred for 4 hr at 70 degrees C. in an oil bath. The reaction mixture was cooled to room temperature. The resulting mixture was concentrated under vacuum. The resulting solution was diluted with 100 mL of water. The pH value of the solution was adjusted to 6-7 with HCl (2 mol/L). The resulting solution was extracted with 3×100 mL of ethyl acetate and the organic layers combined. The resulting mixture was washed with 1×500 ml of brine. The mixture was dried over anhydrous sodium sulfate and concentrated under vacuum. The crude product was re-crystallized from EtOAc:PE in the ratio of 1:10. The solids were collected by filtration. This resulted in 415 mg (60.45%) of 2-[7-[(tert-butoxycarbonyl)(methyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoic acid as a yellow solid.LC-MS (ES, m/z): M+1=716.

Synthesis of tert-butyl N-[1-[5-(4-[[2-(4-chlorophenyl)-4,4-dimethylcyclohex-1-en-1-yl]methyl]piperazin-1-yl)-2-[(4-[[(2S)-1,4-dioxan-2-ylmethyl]amino]-3-nitrobenzenesulfonyl)carbamoyl]phenyl]-2H,3H,4H-pyrido[2,3-b][1,4]oxazepin-7-yl]-N-methylcarbamate: Into a 40-mL vial, was placed 2-[7-[(tert-butoxycarbonyl)(methyl)amino]-2H,3H,4H-pyrido[2,3-b][1,4]oxazepin-1-yl]-4-(4-[[2-(4-chlorophenyl)-4,4-dimethylcyclohex-1-en-1-yl]methyl]piperazin-1-yl)benzoic acid (200.00 mg, 0.279 mmol, 1.00 equiv), DCM (5.00 mL), 4-[[(2S)-1,4-dioxan-2-ylmethyl]amino]-3-nitrobenzenesulfonamide (89.00 mg, 0.280 mmol, 1.00 equiv), EDCI (108.00 mg, 0.563 mmol, 2.02 equiv), DMAP (137.00 mg, 1.121 mmol, 4.02 equiv). The resulting solution was stirred overnight at 30 degrees C. in an oil bath. The reaction mixture was cooled to room temperature. The resulting mixture was concentrated under vacuum. The residue was applied onto a silica gel column with dichloromethane/methanol (10:1). This resulted in 120 mg (42.32%) of tert-butyl N-[1-[5-(4-[[2-(4-chlorophenyl)-4,4-dimethylcyclohex-1-en-1-yl]methyl]piperazin-1-yl)-2-[(4-[[(2S)-1,4-dioxan-2-ylmethyl]amino]-3-nitrobenzenesulfonyl)carbamoyl]phenyl]-2H,3H,4H-pyrido[2,3-b][1,4]oxazepin-7-yl]-N-methylcarbamate as a yellow solid. LC-MS (ES, m/z): M+1=1015.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[7-(methylamino)-2H,3H,4H-pyrido[2,3-b][1,4]oxazepin-1-yl]benzamide hydrochloride: Into a 50-mL round-bottom flask, was placed tert-butyl N-[1-[5-(4-[[2-(4-chlorophenyl)-4,4-dimethylcyclohex-1-en-1-yl]methyl]piperazin-1-yl)-2-[(4-[[(2S)-1,4-dioxan-2-ylmethyl]amino]-3-nitrobenzenesulfonyl)carbamoyl]phenyl]-2H,3H,4H-pyrido[2,3-b][1,4]oxazepin-7-yl]-N-methylcarbamate (120.00 mg, 0.118 mmol, 1.00 equiv), HCl (gas) in 1,4-dioxane (5.00 mL). The resulting solution was stirred for 4 hr at 25 degrees C. The resulting mixture was concentrated under vacuum. The pH value of the solution was adjusted to 8 with NH₃.H₂O. The crude product was purified by Prep-HPLC with the following conditions (Prep-HPLC-006): Column, XBridge Shield RP18 OBD Column, 5 um, 19*150 mm; mobile phase, Water (0.05% FA) and ACN (40% Phase B up to 70% in 7 min); Detector, UV 254/220 nm. The collected solution was concentrated under vacuum to remove CH₃CN and the resulting solution was dried by lyophilization (added with Conc.HCl (1 drop)). This resulted in 20 mg (18.49%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[7-(methylamino)-2H,3H,4H-pyrido[2,3-b][1,4]oxazepin-1-yl]benzamide hydrochloride as a yellow solid. LC-MS (ES, m/z): M-HCl+1=915, ¹H NMR (300 MHz, DMSO-d₆, ppm) δ11.95 (s, 1H), 10.87 (s, 1H), 8.63 (s, 1H), 8.55 (d, J=2.1 Hz, 1H), 7.93-7.76 (m, 1H), 7.42 (d, J=7.9 Hz, 2H), 7.29 (d, J=8.7 Hz, 1H), 7.22 (d, J=9.3 Hz, 1H), 7.14 (d, J=7.9 Hz, 2H), 6.91 (s, 1H), 6.59 (d, J=7.9 Hz, 2H), 6.12 (s, 1H), 4.41 (s, 2H), 4.08-3.06 (m, 19H), 2.73 (s, 5H), 2.41 (s, 2H), 2.01 (d, J=21.8 Hz, 4H), 1.47 (s, 2H), 0.97 (s, 6H).

### Compound 3-16: Preparation of 4-(4-[[2-(4-chlorophenyl)-4,4-dimethylcyclohex-1-en-1-yl]methyl]piperazin-1-yl)-N- [(3S or 3R)-5-nitro-3-(oxan-4-yl)-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide & 4-(4-[[2-(4-chlorophenyl)-4,4-dimethylcyclohex-1-en-1-yl]methyl]piperazin-1-yl)-N-[(3R or 3S)-5-nitro-3-(oxan-4-yl)-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide

Synthesis of 4-(4-[[2-(4-chlorophenyl)-4,4-dimethylcyclohex-1-en-1-yl]methyl]piperazin-1-yl) -N-[(3S)-5-nitro-3-(oxan-4-yl)-3,4-dihydro-2H-1,4-benzoxazin-7-ylsulfonyl]-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide(Assumed) Into a 8-mL vial, was placed 4-(4-[[2-(4-chlorophenyl)-4,4-dimethylcyclohex-1-en-1-yl]methyl]iperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (25.00 mg, 0.033 mmol, 1.00 equiv), (3S)-5-nitro-3-(oxan-4-yl)-3,4-dihydro-2H-1,4-benzoxazine-7-sulfonamide (11.35 mg, 0.033 mmol, 1.00 equiv), EDCI (12.67 mg, 0.066 mmol, 2.00 equiv), DMAP (12.11 mg, 0.099 mmol, 3.00 equiv), DCM (1.00 mL). The resulting solution was stirred for 14 hr at 35 degrees C. in an oil bath. The resulting mixture was concentrated. The residue was purified by Prep-TLC with dichloromethane/methanol (10:1). This resulted in 22 mg (61.53%) of 4-(4-[[2-(4-chlorophenyl)-4,4-dimethylcyclohex-1-en-1-yl]methyl]piperazin-1-yl)-N-[(3S)-5-nitro-3-(oxan-4-yl)-3,4-dihydro-2H-1,4-benzoxazin-7-ylsulfonyl]-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide as a yellow solid. LC-MS (ES, m/z): 1081 [M+H].

Synthesis of 4-(4-[[2-(4-chlorophenyl)-4,4-dimethylcyclohex-1-en-1-yl]methyl]piperazin-1-yl)-N- [(3S)-5-nitro-3-(oxan-4-yl)-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide Into a 8-mL vial, was placed 4-(4-[[2-(4-chlorophenyl)-4,4-dimethylcyclohex-1-en-1-yl]methyl]pipera-zin-1-yl)-N-[(3S)-5-nitro-3-(oxan-4-yl)-3,4-dihydro-2H-1,4-benzoxazin-7-ylsulfonyl]-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide (20.00 mg, 0.018 mmol, 1.00 equiv), ethane-1,2-diamine (0.20 mL), TBAF in THF(1 ml, 2M). The resulting solution was stirred for 48 hr at 70 degrees C. in an oil bath. The resulting mixture was concentrated. The residue was purified by Prep-TLC with dichloromethane/methanol (10:1).The crude product was purified by Prep-HPLC with the following conditions: Column, X Bridge Prep C18 OBD 19*150 mm 5 um; mobile phase, A: 0.1% NH₃-H₂O in water; B: ACN; Gradient: 6-85% B in 7.9 min; Flow rate: 20 ml/min; Detector, 220 nm. This resulted in 6 mg (34.11%) of 4-(4-[[2-(4-chlorophenyl)-4,4-dimethylcyclohex-1-en-1-yl]methyl]piperazin-1-yl)-N-[(3S)-5-nitro-3-(oxan-4-yl)-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide as a yellow solid. LC-MS (ES, m/z): 951.4 [M+H]¹H NMR (300 MHz, CDCl₃, ppm): δ12.22 (s, 1H), 8.73 (s, 1H), 8.47 (d, J=2.1 Hz, 1H), 8.37 (s, 1H), 8.00 (d, J=9.6 Hz, 1H), 7.50 (s, 1H), 7.27 (s, 1H), 7.18 (s, 1H), 7.07 (s, 1H), 7.02-6.98 (m, 2H), 6.76-6.72 (m, 2H), 6.25 (s, 1H), 4.40 (s, 1H), 4.10-3.90 (m, 4H), 3.78-3.20 (m, 9H), 2.85 (s, 2H), 2.45-2.14 (m, 6H), 2.03 (s, 2H), 1.90-1.32 (m, 10H), 0.96 (s, 6H).

Synthesis of 4-(4-[[2-(4-chlorophenyl)-4,4-dimethylcyclohex-1-en-1-yl]methyl]piperazin-1-yl)-N- [(3R)-5-nitro-3-(oxan-4-yl)-3,4-dihydro-2H-1,4-benzoxazin-7-ylsulfonyl]-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide. Into a 8-mL vial, was placed 4-(4-[[2-(4-chlorophenyl)-4,4-dimethylcyclohex-1-en-1-yl]methyl]pipera -zin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (25.00 mg, 0.033 mmol, 1.00 equiv), (3R)-5-nitro-3-(oxan-4-yl)-3,4-dihydro-2H-1,4-benzoxazine-7-sulfonamide (11.35 mg, 0.033 mmol, 1.00 equiv), EDCI (12.67 mg, 0.066 mmol, 2.00 equiv), DMAP (12.11 mg, 0.099 mmol, 3.00 equiv), DCM (1.00 mL). The resulting solution was stirred for 14 hr at 35 degrees C. in an oil bath. The resulting mixture was concentrated. The residue was purified with Prep-TLC with dichloromethane/methanol (10:1). This resulted in 20 mg (55.94%) of 4-(4-[[2-(4-chlorophenyl)-4,4-dimethylcyclohex-1-en-1-yl]methyl]piperazin-1-yl)-N- [(3R)-5-nitro-3-(oxan-4-yl)-3,4-dihydro-2H-1,4-benzoxazin-7-ylsulfonyl]-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide as a yellow solid. LC-MS (ES, m/z): 1081 [M+H].

Synthesis of 4-(4-[[2-(4-chlorophenyl)-4,4-dimethylcyclohex-1-en-1-yl]methyl]piperazin-1-yl)-N- [(3R)-5-nitro-3-(oxan-4-yl)-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide Into a 8-mL vial, was placed 4-(4-[[2-(4-chlorophenyl)-4,4-dimethylcyclohex-1-en-1-yl]methyl]pipera-zin-1-yl)-N-[(3R)-5-nitro-3-(oxan-4-yl)-3,4-dihydro-2H-1,4-benzoxazin-7-ylsulfonyl]-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide (18.00 mg, 1 equiv), ethane-1,2-diamine (0.20 mL), TBAF in THF(1 ml, 2M). The resulting solution was stirred for 48 hr at 70 degrees C. in an oil bath. The resulting mixture was concentrated. The residue was purified by Prep-TLC with dichloromethane/methanol (10:1).The crude product was purified by Prep-HPLC with the following conditions: Column, X Bridge Prep C18 OBD 19*150 mm 5 um; mobile phase, A: 0.1% NH₃-H₂O in water; B: ACN; Gradient: 6-80% B in 7.9 min; Flow rate: 20 ml/min; Detector, 220 nm. This resulted in 7 mg (44.21%) of 4-(4-[[2-(4-chlorophenyl)-4,4-dimethylcyclohex-1-en-1-yl]methyl]piperazin-1-yl)-N-[(3R)-5-nitro-3-(oxan-4-yl)-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide as a yellow solid. LC-MS-PH-PHNW-4-141-0: (ES, m/z): 951.4 [M+H]¹H NMR- PH-PHNW-4-141-0 (300 MHz, CDCl₃, ppm): δ12.23 (s, 1H), 8.68 (s, 1H), 8.48 (d, J=2.1 Hz, 1H), 8.37 (s, 1H), 8.00 (d, J=9.6 Hz, 1H), 7.51 (s, 1H), 7.27 (s, 1H), 7.17 (s, 1H), 7.07 (s, 1H), 7.02-6.98 (m, z, 2H), 6.77-6.71 (m, 2H), 6.25 (s, 1H), 4.40 (s, 1H), 4.10-3.90 (m, 4H), 3.78-3.20 (m, 9H), 2.45-2.13 (m, 6H), 2.03 (s, 2H), 1.89-1.37 (m, 10H), 0.98 (s, 6H).

### Compound 3-17: Preparation of 4-(4-[[2-(4-chlorophenyl)-4,4-dimethylcyclohex-1-en-1-yl]methyl]piperazin-1-yl)-N-[(3S or 3R)-3-[(2S)-1,4-dioxan-2-yl]-5-nitro-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide & 4-(4-[[2-(4-chlorophenyl)-4,4-dimethylcyclohex-1-en-1-yl]methyl]piperazin-1-yl)-N-[(3R or 3S)-3-[(2S)-1,4-dioxan-2-yl]-5-nitro-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide

Synthesis of (2R)-1,4-dioxane-2-carbaldehyde, Into a 500-mL round-bottom flask, was placed (2S)-1,4-dioxan-2-ylmethanol (30.00 g, 253.953 mmol, 1.00equiv), CH₃CN (250.00 mL), IBX (120.89 g, 431.724 mmol, 1.70equiv). The resulting solution was stirred for 4 h at 70 degrees C. in an oil bath. The solids were filtered out. The resulting mixture was concentrated. This resulted in 30 g (crude) of (2R)-1,4-dioxane-2-carbaldehyde as colorless oil. H-NMR-1: ¹H NMR (300 MHz, CDCl₃, ppm) δ9.66 (s, 1H), 4.12-3.77 (m, 7H).

Synthesis of 2-amino-2-[(2S)-1,4-dioxan-2-yl]acetonitrile, Into a 500-mL pressure tank reactor, was placed (2R)-1,4-dioxane-2-carbaldehyde (30.00 g, 258.362 mmol, 1.00equiv). NH₃/MeOH(7M) (300.00 mL). This was followed by the addition of TMSCN (38.40 g, 387.071 mmol, 1.50equiv). The resulting solution was stirred for overnight at 70 degrees C. in an oil bath. The resulting mixture was concentrated. The residue was applied onto a silica gel column and eluted with ethyl acetate/petroleum ether (3:1). The collected fractions were combined and concentrated. This resulted in 18 g (49.01%) of (2S)-2-amino-2-[(2S)-1,4-dioxan-2-yl]acetonitrile as yellow oil. LCMS-2 (ES, m/z): M+1: 143.

Synthesis of amino((2S)-1,4-dioxan-2-yl)acetic acid: Into a 100-mL round-bottom flask, was placed 2-amino-2-[(2S)-1,4-dioxan-2-yl]acetonitrile (18.00 g, 126.620 mmol, 1.00equiv), NaOH(4M) (40.00 mL). The resulting solution was stirred for overnight at 70 degrees C. in an oil bath. The pH value of the solution was adjusted to 6 with HOAc. The solids were collected by filtration. The solid was dried in an oven under reduced pressure.

This resulted in 8.8 g (43.13%) of amino((2S)-1,4-dioxan-2-yl)acetic acid as a white solid LCMS-3 (ES, m/z): M+1: 162.

Synthesis of 2-amino-2-[(2S)-1,4-dioxan-2-yl]ethanol: Into a 250-mL round-bottom flask, was placed amino((2S)-1,4-dioxan-2-yl)acetic acid (8.80 g, 54.605 mmol, 1.00equiv), THE (120.00 mL, 1.664 mmol, 0.03equiv). This was followed by the addition of LiAlH₄ (6.22 g, 163.883 mmol, 3.00equiv) in several batches at 0 degrees C. The resulting solution was stirred for 6 h at 60 degrees C. in an oil bath. The reaction was then quenched by the addition of 12 g of Na₂SO₄.10H₂O. The solids were filtered out. The resulting mixture was concentrated. This resulted in 4.5 g (55.99%) of 2-amino-2-[(2S)-1,4-dioxan-2-yl]ethanol as a white solid. LCMS-4 (ES, m/z): M+1: 148.

Synthesis of 3-bromo-4-([1-[(2S)-1,4-dioxan-2-yl]-2-hydroxyethyl]amino)-5-nitrobenzenesulfonamide: Into a 250-mL round-bottom flask, was placed 3-bromo-4-chloro-5-nitrobenzenesulfonamide (4.00 g, 12.677 mmol, 1.00equiv), CH₃CN (120.00 mL), DIEA (6.55 g, 0.051 mmol, 4equiv), 2-amino-2-[(2S)-1,4-dioxan-2-yl]ethanol (4.48 g, 0.030 mmol, 2.4equiv). The resulting solution was stirred for 48 h at 80 degrees C. in an oil bath. The resulting mixture was concentrated. The residue was applied onto a silica gel column and eluted with ethyl acetate/petroleum ether (9:1). The collected fractions were combined and concentrated. This resulted in 3.2 g (59.22%) of 3-bromo-4-([1-[(2S)-1,4-dioxan-2-yl]-2-hydroxyethyl]amino)-5-nitrobenzenesulfonamide as a yellow solid. LCMS-5 (ES, m/z): M-1: 424.

Synthesis of (3S or 3R)-3-[(2S)-1,4-dioxan-2-yl]-5-nitro-3,4-dihydro-2H-1,4-benzoxazine-7-sulfonamide& (3R or 3S)-3-[(2S)-1,4-dioxan-2-yl]-5-nitro-3,4-dihydro-2H-1,4-benzoxazine-7-sulfonamide: Into a 100-mL round-bottom flask purged and maintained with an inert atmosphere of nitrogen, was placed 3-bromo-4-([1-[(2S)-1,4-dioxan-2-yl]-2-hydroxyethyl]amino)-5-nitrobenzenesulfonamide (1.00 g, 2.346 mmol, 1.00equiv), dioxane (40.00 mL), Cs₂CO₃ (1911.01 mg, 5.865 mmol, 2.50equiv), t-BuXPhos Pd G3 (186.05 mg, 0.235 mmol, 0.10equiv). The resulting solution was stirred for 8 h at 100 degrees C. in an oil bath. The reaction was then quenched by the addition of 40 mL of water. The resulting solution was extracted with 3×40 mL of ethyl acetate dried over anhydrous sodium sulfate and concentrated. The residue was applied onto a silica gel column and eluted with ethyl acetate/petroleum ether (1:1). The collected fractions were combined and concentrated. The crude product was purified by Prep-HPLC with the following conditions: Column, X-bridge RP18; mobile phase, 0.05% ammonia in water and CH₃CN (45% CH₃CN up to 60% in 5 min); Detector, UV 254 nm. This resulted in 20 mg (2.47%) of (3S or 3R)-3-[(2S)-1,4-dioxan-2-yl]-5-nitro-3,4-dihydro-2H-1,4-benzoxazine-7-sulfonamide as a yellow solid and 40 mg (4.94%) of (3R or 3S)-3-[(2S)-1,4-dioxan-2-yl]-5-nitro-3,4-dihydro-2H-1,4-benzoxazine-7-sulfonamide as a yellow solid LC-MS-6: (ES, m/z): M-1:344.

Synthesis of 4-(4-[[2-(4-chlorophenyl)-4,4-dimethylcyclohex-1-en-1-yl]methyl]piperazin-1-yl)-N-[(3S or 3R)-3-[(2S)-1,4-dioxan-2-yl]-5-nitro-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide: Into a 8-mL vial, was placed 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid (18.13 mg, 0.029 mmol, 1 equiv), (3S or 3R)-3-[(2S)-1,4-dioxan-2-yl]-5-nitro-3,4-dihydro-2H-1,4-benzoxazine-7-sulfonamide (10.00 mg, 0.029 mmol, 1.00equiv), DCM (3.00 mL), EDCI (11.10 mg, 0.058 mmol, 2.00equiv), DMAP (14.15 mg, 0.116 mmol, 4.00equiv). The resulting solution was stirred for overnight at room temperature. The resulting mixture was concentrated. The crude product was purified by Prep-HPLC with the following conditions: Column, X-bridge RP18; mobile phase, 0.05% ammonia in water and CH₃CN (45% CH₃CN up to 60% in 5 min); Detector, UV 254 nm. This resulted in 3.2 mg (11.59%) of 4-(4-[[2-(4-chlorophenyl)-4,4-dimethylcyclohex-1-en-1-yl]methyl]piperazin-1-yl)-N-[(3S or 3R)-3-[(2S)-1,4-dioxan-2-yl]-5-nitro-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide as a yellow solid. LC-MS-0: (ES, m/z): M+1: 953. ¹H NMR (300 MHz, Chloroform-d, ppm) δ12.09 (d, J=26.1 Hz, 1H), 8.61 (s, 1H), 8.50 (s, 1H), 8.23 (s, 1H), 7.99 (d, J=8.4 Hz, 1H), 7.63 (s, 1H), 7.35 (s, 2H), 7.16 (s, 1H), 7.00 (d, J=8.4 Hz, 3H), 6.74 (d, J=8.1 Hz, 2H), 6.25 (d, J=15.9 Hz, 1H), 4.38 (s, 1H), 4.00-3.37 (m, 14H), 3.31 (s, 2H), 2.86 (s, 1H), 2.63 (s, 2H), 2.32 (d, J=35.6 Hz, 5H), 2.08 (d, J=25.8 Hz, 2H), 1.48 (s, 1H), 1.28 (s, 2H), 1.01 (s, 6H).

Synthesis of 4-(4-[[2-(4-chlorophenyl)-4,4-dimethylcyclohex-1-en-1-yl]methyl]piperazin-1-yl)-N-[(3R or 3S)-3-[(2S)-1,4-dioxan-2-yl]-5-nitro-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide: Into a 8-mL vial, was placed 4-(4-[[2-(4-chlorophenyl)-4,4-dimethylcyclohex-1-en-1-yl]methyl]piperazin-1-yl)-2-[14-oxa-2,4,10-triazatricyclo[7.5.0.0\hat{ }[3,7]]tetradeca-1(9),2,5,7-tetraen-10-yl]benzoic acid (18.13 mg, 0.029 mmol, 1 equiv), (3S or 3S)-3-[(2S)-1,4-dioxan-2-yl]-5-nitro-3,4-dihydro-2H-1,4-benzoxazine-7-sulfonamide (10.00 mg, 0.029 mmol, 1.00equiv), DCM (3.00 mL), EDCI (11.10 mg, 0.058 mmol, 2.00equiv), DMAP (14.15 mg, 0.116 mmol, 4.00equiv). The resulting solution was stirred for overnight at room temperature. The resulting mixture was concentrated. The crude product was purified by Prep-HPLC with the following conditions: Column, X-bridge RP18; mobile phase, 0.05% ammonia in water and CH₃CN (45% CH₃CN up to 60% in 5 min); Detector, UV 254 nm. This resulted in 3.1 mg (11.23%) of 4-(4-[[2-(4-chlorophenyl)-4,4-dimethylcyclohex-1-en-1-yl]methyl]piperazin-1-yl)-N-[(3R or 3S)-3-[(2S)-1,4-dioxan-2-yl]-5-nitro-3,4-dihydro-2H-1,4-benzoxazin-7-ylsulfonyl]-2-[14-oxa-2,4,10-triazatricyclo[7.5.0.0\hat{ }[3,7]]tetradeca-1(9),2,5,7-tetraen-10-yl]benzamide as a yellow solid.LC-MS-0: (ES, m/z): M+1: 953. ¹H NMR (300 MHz, Chloroform-d, ppm) δ12.05 (d, J=29.4 Hz, 1H), 8.82 (s, 1H), 8.49 (s, 2H), 7.98 (d, J=9.0 Hz, 1H), 7.58 (s, 1H), 7.36 (s, 1H), 7.17 (s, 1H), 7.00 (d, J=7.5 Hz, 3H), 6.74 (d, J=8.1 Hz, 2H), 6.25 (d, J=14.4 Hz, 1H), 4.15-3.44 (m, 16H), 3.31 (s, 2H), 2.86 (s, 1H), 2.64 (s, 2H), 2.32 (d, J=37.5 Hz, 4H), 2.08 (d, J=25.3 Hz, 2H), 1.50 (s, 4H), 1.28 (s, 1H), 1.01 (s, 6H).

### Compound 3-30: Synthesis of (R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide

Into a 50-mL round-bottom flask, was placed a solution of 4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide (15 mg, 0.015 mmol, 1.00 equiv) in methanol (5 mL). The resulting solution was purified by Chiral-Prep-HPLC with the following conditions (SHIMADZU LC-20AT): Column, CHIRALPAK ID-3, 4.6*50 mm, 3 m; mobile phase A: n-Hexane/DCM=5/1; Phase B: EtOH/MeOH=1/1; Detector, PDA. This resulted in 2.5 mg (33%) of (R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide as a yellow solid. LC-MS: (ES, m/z): M+1=991, R,T=1.61 min. The measurements of the retention were done with a reversed phase column (C18). Shimadzu LCMS 2020; 50*3.0 Agilent Poroshell HPH-C18, 2.7 um; Eluent A: water (0.05% ammonia water); Eluent B: Acetonitrile; linear gradient from 5% acetonitrile to 95% acetonitrile in 7.0 minutes; Oven temperature 40° C.; flow: 1.5 mL/min. ¹H NMR (300 MHz, CDCL3, ppm) δ11.43 (ds, 1H), 9.99 (ds, 1H), 8.72 (s, 1H), 8.40 (ds, 1H), 7.93-7.85 (m, 2H), 7.37-7.35 (m, 2H), 7.26 (s, 1H), 7.02-6.93 (m, 3H), 6.71-6..64 (m, 3H), 6.27 (s, 1H), 4.58 (s, 1H), 4.05-3.15 (m, 23H), 2.70-2.35 (m, 7H), 2.03 (s, 3H), 1.92-1.90 (m, 1H), 1.89-1.87 (m, 2H), 1.71-1.67 (m, 2H), 1.53-1.45 (m, 3H), 1.00 (s, 6H).

### Compound 3-31: Synthesis of (S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide

Into a 50-mL round-bottom flask, was placed a solution of 4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide (15 mg, 0.015 mmol, 1.00 equiv) in methanol (5 mL). The resulting solution was purified by Chiral-Prep-HPLC with the following conditions (SHIMADZU LC-20AT): Column, CHIRALPAK ID-3, 4.6*50 mm, 3 m; mobile phase A: n-Hexane/DCM=5/1; Phase B: EtOH/MeOH=1/1; Detector, PDA. This resulted in 2.5 mg (33%) of (S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide as a yellow solid LC-MS: (ES, m/z): M+1=991, R,T=1.61 min. The measurements of the retention were done with a reversed phase column (C18). Shimadzu LCMS 2020; 50*3.0 Agilent Poroshell HPH-C18, 2.7 um; Eluent A: water (0.05% ammonia water); Eluent B: Acetonitrile; linear gradient from 5% acetonitrile to 95% acetonitrile in 7.0 minutes; Oven temperature 40° C.; flow: 1.5 mL/min. ¹H NMR (300 MHz, CDCL3, ppm) δ11.43 (ds, 1H), 9.99 (ds, 1H), 8.72 (s, 1H), 8.40 (ds, 1H), 7.93-7.85 (m, 2H), 7.37-7.35 (m, 2H), 7.26 (s, 1H), 7.02-6.93 (m, 3H), 6.71-6..64 (m, 3H), 6.27 (s, 1H), 4.58 (s, 1H), 4.05-3.15 (m, 23H), 2.70-2.35 (m, 7H), 2.03 (s, 3H), 1.92-1.90 (m, 1H), 1.89-1.87 (m, 2H), 1.71-1.67 (m, 2H), 1.53-1.45 (m, 3H), 1.00 (s, 6H).

### Compound 3-32: Preparation of 4-(4-[[2-(4-chlorophenyl)-4,4-dimethylcyclohex-1-en-1-yl]methyl]piperazin-1-yl)-2-[13,13-difluoro-14-oxa-2,4,10-triazatricyclo[7.5.0.0\hat{ }[3,7]]tetradeca-1(9),2,5,7-tetraen-10-yl]-N-(4-[[(2R)-1,4-dioxan-2-ylmethyl]amino]-2-nitrobenzenesulfonyl)benzamide hydrochloride

Synthesis of 1-bromo-3,3-diethoxy-1,1-difluoropropane: Into a 250-mL sealed tube, was placed ethoxyethene (14.40 g, 199.703 mmol, 1.00equiv), EtOH (80.00 mL), dibromodifluoromethane (62.85 g, 0.300 mmol, 1.5equiv), Na₂S₂O₄ (52.15 g, 0.300 mmol, 1.5equiv), NaHCO₃(50.33 g, 0.599 mmol, 3equiv). The resulting solution was stirred for 4 h at 60 degrees C. in an oil bath. The reaction was then quenched by the addition of 200 mL of water/ice. The resulting solution was extracted with 3×200 mL of petroleum ether. The resulting mixture was washed with 2×100 ml of water. The mixture was dried over anhydrous sodium sulfate. The solids were filtered out. The resulting mixture was concentrated under vacuum. This resulted in 12 g (24.32%) of 1-bromo-3,3-diethoxy-1,1-difluoropropane as yellow oil. ¹H NMR (300 MHz, DMSO-d₆): δ 4.82 (t, J=5.2 Hz, 1H), 3.7-3.55 (m, 2H), 3.54-3.43 (m, 2H), 2.83 (td, J=14.7, 5.1 Hz, 2H), 1.13 (t, J=7.2 Hz, 6H).

Synthesis of ethyl 3-bromo-3,3-difluoropropanoate: Into a 250-mL round-bottom flask, was placed 1-bromo-3,3-diethoxy-1,1-difluoropropane (12.00 g, 48.567 mmol, 1.00 equiv), DCM (120.00 mL), m-CPBA (11.73 g, 0.068 mmol, 1.4 quiv), H₂SO₄ (0.10 g, 0.001 mmol, 0.02equiv). The resulting solution was stirred for overnight at 55 degrees C. in an oil bath. The solids were filtered out. The pH value of the solution was adjusted to 8 with NaHCO₃(5 mol/L). The resulting solution was extracted with 2×120 mL of dichloromethane. The solution was concentrated under vacuum. This resulted in 8 g (75.90%) of ethyl 3-bromo-3,3-difluoropropanoate as light yellow oil.

Synthesis of 5-bromo-1-[[2-(trimethylsilyl)ethoxy]methyl]-1H-pyrrolo[2,3-b]pyridin-6-ol: Into a 100-mL round-bottom flask, was placed 5-bromo-6-fluoro-1-[[2-(trimethylsilyl)ethoxy]methyl]-1H-pyrrolo[2,3-b]pyridine (3.00 g, 8.688 mmol, 1.00 quiv), NMP (50.00 mL), H₂O (5.00 mL), KOH (1.46 g, 0.026 mmol, 3equiv). The resulting solution was stirred for 3 h at 135 degrees C. in an oil bath. The reaction was then quenched by the addition of 50 mL of water. The resulting solution was extracted with 4×50 mL of ethyl acetate and concentrated under vacuum. The residue was applied onto a silica gel column and eluted with ethyl acetate/petroleum ether (1:5). The collected fractions were combined and concentrated. This resulted in 1.5 g (50.29%) of 5-bromo-1-[[2-(trimethylsilyl)ethoxy]methyl]-1H-pyrrolo[2,3-b]pyridin-6-ol as a white solid. LCMS (ES, m/z): M+1: 343/345.

Synthesis of ethyl 3-[(5-bromo-1-[[2-(trimethylsilyl)ethoxy]methyl]-1H-pyrrolo[2,3-b]pyridin-6-yl)oxy]-3,3-difluoropropanoate: Into a 50-mL round-bottom flask, was placed 5-bromo-1-[[2-(trimethylsilyl)ethoxy]methyl]-1H-pyrrolo[2,3-b]pyridin-6-ol (1.02 g, 2.971 mmol, 1.00equiv), DMF (10.00 mL), TEA (0.75 g, 0.007 mmol, 2.5equiv), ethyl 3-bromo-3,3-difluoropropanoate (0.64 g, 0.003 mmol, 1 equiv). The resulting solution was stirred for overnight at 0 degrees C. to room temperature. The reaction was then quenched by the addition of 20 mL of water. The resulting solution was extracted with 3×20 mL of ethyl acetate and concentrated. The residue was applied onto a silica gel column and eluted with ethyl acetate/petroleum ether (1:10). The collected fractions were combined and concentrated. This resulted in 1.6 g (112.33%) of ethyl 3-[(5-bromo-1-[[2-(trimethylsilyl)ethoxy]methyl]-1H-pyrrolo[2,3-b]pyridin-6-yl)oxy]-3,3-difluoropropanoate as yellow oil. LC-MS: (ES, m/z): M+1:479/481.

Synthesis of 3-[(5-bromo-1-[[2-(trimethylsilyl)ethoxy]methyl]-1H-pyrrolo[2,3-b]pyridin-6-yl)oxy]-3,3-difluoropropan-1-ol: Into a 100-mL 3-necked round-bottom flask purged and maintained with an inert atmosphere of nitrogen, was placed ethyl 3-[(5-bromo-1-[[2-(trimethylsilyl)ethoxy]methyl]-1H-pyrrolo[2,3-b]pyridin-6-yl)oxy]-3,3-difluoropropanoate (1.60 g, 3.338 mmol, 1.00equiv), THE (20.00 mL), LiAlH₄ (0.51 g, 0.013 mmol, 4equiv). The resulting solution was stirred for 2h at -78- to ˜30 degrees C. The reaction was then quenched by the addition of 20 mL of water. The solids were filtered out.

The resulting solution was extracted with 3×20 mL of ethyl acetate and concentrated. The residue was applied onto a silica gel column and eluted with ethyl acetate/petroleum ether (1:4). The collected fractions were combined and concentrated under vacuum. This resulted in 900 mg (61.66%) of 3-[(5-bromo-1-[[2-(trimethylsilyl)ethoxy]methyl]-1H-pyrrolo[2,3-b]pyridin-6-yl)oxy]-3,3-difluoropropan-1-ol as light yellow oil. LC-MS: (ES, m/z): M+1:437/439.

Synthesis of 3-[(5-bromo-1-[[2-(trimethylsilyl)ethoxy]methyl]-1H-pyrrolo[2,3-b]pyridin-6-yl)oxy]-3,3-difluoropropyl methanesulfonate: Into a 100-mL round-bottom flask, was placed 3-[(5-bromo-1-[[2-(trimethylsilyl)ethoxy]methyl]-1H-pyrrolo[2,3-b]pyridin-6-yl)oxy]-3,3-difluoropropan-1-ol (900.00 mg, 2.058 mmol, 1.00equiv), DCM (20.00 mL), TEA (624.69 mg, 6.173 mmol, 3equiv), methanesulfonyl chloride (259.27 mg, 2.26 mmol, 1.10 equiv). The resulting solution was stirred for 3 h at 0 degrees C. to room temperature. The reaction was then quenched by the addition of 20 mL of water. The resulting solution was extracted with 3×20 mL of ethyl acetate and concentrated. The residue was applied onto a silica gel column and eluted with ethyl acetate/petroleum ether (1:5). The collected fractions were combined and concentrated under vacuum. This resulted in 1 g (94.28%) of 3-[(5-bromo-1-[[2-(trimethylsilyl)ethoxy]methyl]-1H-pyrrolo[2,3-b]pyridin-6-yl)oxy]-3,3-difluoropropyl methanesulfonate as light yellow oil. LC-MS: (ES, m/z): M+1:515/517.

Synthesis of 3-[(5-bromo-1-[[2-(trimethylsilyl)ethoxy]methyl]-1H-pyrrolo[2,3-b]pyridin-6-yl)oxy]-3,3-difluoropropan-1-amine: Into a 50-mL sealed tube, was placed 3-[(5-bromo-1-[[2-(trimethylsilyl)ethoxy]methyl]-1H-pyrrolo[2,3-b]pyridin-6-yl)oxy]-3,3-difluoropropyl methanesulfonate (1.00 g, 1.94 mmol, 1.00equiv). NH₃/MeOH(7M) (20.00 mL). The resulting solution was stirred for overnight at 70 degrees C. in an oil bath. The resulting mixture was concentrated under vacuum. This resulted in 600 mg (70.87%) of 3-[(5-bromo-1-[[2-(trimethylsilyl)ethoxy]methyl]-1H-pyrrolo[2,3-b]pyridin-6-yl)oxy]-3,3-difluoropropan-1-amine as yellow oil. LC-MS: (ES, m/z): M+1:436/438.

Synthesis of 13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 40-mL vial purged and maintained with an inert atmosphere of nitrogen, was placed 3-[(5-bromo-1-[[2-(trimethylsilyl)ethoxy]methyl]-1H-pyrrolo[2,3-b]pyridin-6-yl)oxy]-3,3-difluoropropan-1-amine (600.00 mg, 1.375 mmol, 1.00equiv), toluene (10.00 mL), t-BuONa (396.42 mg, 4.125 mmol, 3equiv), XPhos Pd G3 Precatalyst(116.38 mg, 0.137 mmol, 0.10equiv). The resulting solution was stirred for 2 h at 80 degrees C. in an oil bath. The resulting mixture was concentrated under vacuum. The residue was applied onto a silica gel column and eluted with ethyl acetate/petroleum ether (1:3). The collected fractions were combined and concentrated. This resulted in 400 mg (81.84%) of 13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraene as light yellow oil. LC-MS: (ES, m/z): M+1:356.

Synthesis of methyl 4-(4-[[2-(4-chlorophenyl)-4,4-dimethylcyclohex-1-en-1-yl]methyl]piperazin-1-yl)-2-(13,13-difluoro-4- [[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 100-mL round-bottom flask purged and maintained with an inert atmosphere of nitrogen, was placed 13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraene (400.00 mg, 1.125 mmol, 1.00equiv), toluene (30.00 mL), methyl 2-bromo-4-(4-[[2-(4-chlorophenyl)-4,4-dimethylcyclohex-1-en-1-yl]methyl]piperazin-1-yl)benzoate (838.00 mg, 1.575 mmol, 1.40equiv), Cs₂CO₃ (1099.93 mg, 3.376 mmol, 3equiv), XantPhos Pd G2 Precatalyst(65.11 mg, 0.113 mmol, 0.1 equiv). The resulting solution was stirred for overnight at 110 degrees C. in an oil bath. The solids were filtered out. The resulting mixture was concentrated. The residue was applied onto a silica gel column and eluted with ethyl acetate/petroleum ether (1:3). The collected fractions were combined and concentrated. This resulted in 400 mg (44.08%) of methyl 4-(4-[[2-(4-chlorophenyl)-4,4-dimethylcyclohex-1-en-1-yl]methyl]piperazin-1-yl)-2-(13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as yellow oil. LC-MS: (ES, m/z): M+1:806.

Synthesis of 4-(4-[[2-(4-chlorophenyl)-4,4-dimethylcyclohex-1-en-1-yl]methyl]piperazin-1-yl)-2-(13,13-difluoro-4- [[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid: Into a 40-mL vial, was placed methyl 4-(4-[[2-(4-chlorophenyl)-4,4-dimethylcyclohex-1-en-1-yl]methyl]piperazin-1-yl)-2-(13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (200.00 mg, 0.248 mmol, 1.00equiv), dioxane (2.00 mL), MeOH (2.00 mL), NaOH(4M) (0.40 mL, 1.600 mmol, 6.45equiv). The resulting solution was stirred for overnight at 70 degrees C. in an oil bath. The resulting mixture was concentrated. The pH value of the solution was adjusted to 6 with HCl (2 mol/L). The resulting solution was extracted with 3×20 mL of ethyl acetate concentrated under vacuum. The residue was applied onto a silica gel column and eluted with dichloromethane/methanol (10:1). The collected fractions were combined and concentrated. This resulted in 100 mg (508.85%) of 4-(4-[[2-(4-chlorophenyl)-4,4-dimethylcyclohex-1-en-1-yl]methyl]piperazin-1-yl)-2-(13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid as a white solid. LC-MS: (ES, m/z): M+1:792.

Synthesis of 4-(4-[[2-(4-chlorophenyl)-4,4-dimethylcyclohex-1-en-1-yl]methyl]piperazin-1-yl)-2-(13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)-N-(4-[[(2S)-1,4-dioxan-2-ylmethyl]amino]-3-nitrobenzenesulfonyl)benzamide: Into a 50-mL round-bottom flask, was placed 4-(4-[[2-(4-chlorophenyl)-4,4-dimethylcyclohex-1-en-1-yl]methyl]piperazin-1-yl)-2-(13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (100.00 mg, 0.126 mmol, 1.00equiv), 4-[[(2S)-1,4-dioxan-2-ylmethyl]amino]-3-nitrobenzenesulfonamide (40.04 mg, 0.126 mmol, 1.00equiv), DCM (20.00 mL), EDCI (48.38 mg, 0.25 mmol, 2.00equiv), DMAP (61.67 mg, 0.505 mmol, 4.00equiv). The resulting solution was stirred for overnight at room temperature. The resulting mixture was concentrated. The residue was applied onto a silica gel column and eluted with ethyl acetate/petroleum ether (1:1). The collected fractions were combined and concentrated under vacuum. This resulted in 80 mg (58.07%) of 4-(4-[[2-(4-chlorophenyl)-4,4-dimethylcyclohex-1-en-1-yl]methyl]piperazin-1-yl)-2-(13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)-N-(4-[[(2S)-1,4-dioxan-2-ylmethyl]amino]-3-nitrobenzenesulfonyl)benzamide as a yellow solid. LC-MS: (ES, m/z): M+1:1091.

Synthesis of 4-(4-[[2-(4-chlorophenyl)-4,4-dimethylcyclohex-1-en-1-yl]methyl]piperazin-1-yl)-2-[13,13-difluoro-14-oxa-2,4,10-triazatricyclo[7.5.0.0\hat{ }[3,7]]tetradeca-1(9),2,5,7-tetraen-10-yl]-N-(4-[[(2R)-1,4-dioxan-2-ylmethyl]amino]-2-nitrobenzenesulfonyl)benzamide hydrochloride: Into a 8-mL vial, was placed 4-(4-[[2-(4-chlorophenyl)-4,4-dimethylcyclohex-1-en-1-yl]methyl]piperazin-1-yl)-2-(13,13-difluoro-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)-N-(4-[[(2S)-1,4-dioxan-2-ylmethyl]amino]-3-nitrobenzenesulfonyl)benzamide (50.00 mg, 0.046 mmol, 1.00equiv), THE (5.00 mL), TBAF (119.75 mg, 0.458 mmol, 10.00equiv), ethylene diamine (27.52 mg, 0.458 mmol, 10.00 quiv). The resulting solution was stirred for overnight at 70 degrees C. in an oil bath. The resulting mixture was concentrated. The crude product was purified by Prep-HPLC with the following conditions: Column, X-bridge RP18; mobile phase, 0.05% HCl in water and CH₃CN (45% CH₃CN up to 60% in 5 min); Detector, UV 254 nm. This resulted in 15.1 mg (33.04%) of 4-(4-[[2-(4-chlorophenyl)-4,4-dimethylcyclohex-1-en-1-yl]methyl]piperazin-1-yl)-2-[13,13-difluoro-14-oxa-2,4,10-triazatricyclo[7.5.0.0\hat{ }[3,7]]tetradeca-1(9),2,5,7-tetraen-10-yl]-N-(4-[[(2R)-1,4-dioxan-2-ylmethyl]amino]-2-nitrobenzenesulfonyl)benzamide hydrochloride as a yellow solid. LC-MS: (ES, m/z): M+1-HCl: 961. ¹H NMR (300 MHz, DMSO-d₆) δ 11.68 (s, 1H), 11.43 (s, 1H), 9.73 (s, 1H), 8.60 (t, J=5.7 Hz, 1H), 8.40 (d, J=2.4 Hz, 1H), 7.62 (dd, J=9.3, 2.4 Hz, 1H), 7.48-7.28 (m, 4H), 7.24-7.07 (m, 3H), 7.02 (d, J=9.3 Hz, 1H), 6.70-6.56 (m, 2H), 6.22 (dd, J=3.3, 1.8 Hz, 1H), 3.94-3.74 (m, 5H), 3.71-3.58 (m, 4H), 3.56-3.39 (m, 5H), 3.31 (s, 2H), 3.22 (d, J=12.3 Hz, 2H), 2.89-2.67 (m, 2H), 2.35 (d, J=36.3 Hz, 4H), 2.06 (s, 2H), 1.50 (d, J=7.5 Hz, 2H), 0.97 (s, 6H).

### Compound 3-33: Synthesis of 4-(4-[[2-(4-chlorophenyl)-4,4-dimethylcyclohex-1-en-1-yl]methyl]piperazin-1-yl)-N-[4-([[(2R)-1,4-dioxan-2-yl]methyl]amino)-3-nitrobenzenesulfonyl]-2-[(13R)-13-methyl-14-oxa-2,4,10-triazatricyclo[7.5.0.0\hat{ }[3,7]]tetradeca-1(9),2,5,7-tetraen-10-yl]benzamide(Assumed

### Synthesis of methyl 2-bromo-4-(4-[[2-(4-chlorophenyl)-4,4-dimethylcyclohex-1-en-1-yl]methyl]piperazin-1-yl)benzoate: Into a 250-mL round-bottom flask, was placed a solution of 1-[[2-(4-chlorophenyl)-4,4-dimethylcyclohex-1-en-1-yl]methyl]piperazine (15.09 g, 47.32 mmol, 1.00 equiv) in DMA (150 mL), DIEA (12.9 g, 99.81 mmol, 2.00 equiv), methyl 2-bromo-4-fluorobenzoate (11.6 g, 49.78 mmol, 1.00 equiv). The resulting solution was stirred for 12 h at 100 degree. The reaction mixture was cooled to room temperature. The reaction was then quenched by the addition of 50 mL of water. The resulting solution was extracted with 3×100 mL of ethyl acetate and the organic layers combined. The resulting mixture was washed with 3×100 mL of brine. The mixture was dried over anhydrous sodium sulfate, then filtered and concentrated under vacuum. The residue was applied onto a silica gel column with ethyl acetate/petroleum ether (0:1-1:5). This resulted in 7 g (crude) of methyl 2-bromo-4-(4-[[2-(4-chlorophenyl)-4,4-dimethylcyclohex-1-en-1-yl]methyl]piperazin-1-yl)benzoate as yellow oil. LC-MS: (ES, m/z): M+1=533, 531

Synthesis of 4-([[(2R)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2R)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.8 g (87.14%) of 4-([[(2R)-1,4-dioxan-2-yl]methyl]amino)-3-nitrobenzene-1-sulfonamide as a yellow solid. LC-MS: (ES, m/z): M+1=318, R,T=0.740 min.

Synthesis of N-(3-hydroxybutyl)-4-methylbenzene-1-sulfonamide: Into a 250-mL round-bottom flask purged and maintained with an inert atmosphere of nitrogen, was placed 4-aminobutan-2-ol hydrochloride (4 g, 31.847 mmol, 1 equiv), DCM (40 mL), TEA (3.56 g, 35.181 mmol, 1.10 equiv). This was followed by the addition of 4-methylbenzene-1-sulfonyl chloride (6.08 g, 31.893 mmol, 1.00 equiv) in portions at 0 degrees C. The resulting solution was stirred for 2 h at 25 degrees C. The resulting mixture was concentrated under vacuum. The residue was applied onto a silica gel column with ethyl acetate/petroleum ether (1:1). This resulted in 4.4 g (56.78%) of N-(3-hydroxybutyl)-4-methylbenzene-1-sulfonamide as colorless oil. ¹H NMR (300 MHz, CDCl₃-d,ppm) δ7.82-7.73 (d, J=9.0 Hz, 2H), 7.39-7.30 (d, J=9.0 Hz, 2H), 4.06-3.82 (m, 1H), 3.24-3.16 (m, 1H), 3.06-3.00 (m, 1H), 2.45 (s, 3H), 1.70-1.55 (m, 2H), 1.20 (d, J=6.2 Hz, 3H).

Synthesis of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide: Into a 250-mL 3-necked round-bottom flask purged and maintained with an inert atmosphere of nitrogen, was placed N-(3-hydroxybutyl)-4-methylbenzene-1-sulfonamide (4.4 g, 18.083 mmol, 1.10 equiv), THE (60 mL). This was followed by the addition of NaH (1.97 g, 49.255 mmol, 3.00 equiv, 60%), in portions at 0 degrees C. The resulting solution was stirred for 0.5 h at 0 degrees C. To this was added 5-bromo-6-fluoro-1-[[2-(trimethylsilyl)ethoxy]methyl]-1H-pyrrolo[2,3-b]pyridine (5.66 g, 16.392 mmol, 1 equiv). The resulting solution was stirred overnight at 50 degrees C. in an oil bath. The reaction mixture was cooled to 25 degree C. The reaction was then quenched by the addition of 500 mL of NH₄Cl. The resulting solution was extracted with 2×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 6 g (64.37%) of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide as colorless oil. ¹H NMR (300 MHz, CDCl₃-d,ppm) δ8.04 (s, 1H), 7.74-7.63 (m, 2H), 7.23-7.11 (m, 3H), 6.41 (d, J=3.6 Hz, 1H), 5.56 (d, J=3.3 Hz, 2H), 5.89-5.33 (m, 1H), 3.59-3.46 (m, 2H), 3.17 (t, J=6.0 Hz, 2H), 2.37 (s, 3H), 2.08-1.78 (m, 2H), 1.37 (d, J=6.2 Hz, 3H), 0.94-0.85 (m, 2H), -0.06 (s, 9H).

Synthesis of 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 250-mL round-bottom flask purged and maintained with an inert atmosphere of nitrogen, was placed N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide (6 g, 10.552 mmol, 1 equiv), DMSO (60 mL), pyridine-2-carboxylic acid (1.04 g, 8.448 mmol, 0.80 equiv), CuI (2.41 g, 12.654 mmol, 1.20 equiv), K₂CO₃ (4.38 g, 31.692 mmol, 3.00 equiv). The resulting solution was stirred for 2 days at 125 degrees C. in an oil bath. The reaction mixture was cooled to 25 degree C. The resulting solution was diluted with 500 mL of water. The resulting solution was extracted with 3×200 mL of ethyl acetate and the organic layers combined. The resulting mixture was washed with 2×1000 ml of brine. The mixture was dried over anhydrous sodium sulfate and concentrated under vacuum. The residue was applied onto a silica gel column with ethyl acetate/petroleum ether (1:3). This resulted in 2.8 g (54.41%) of 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, CDCl₃-d,ppm) δ8.10 (s, 1H), 7.53-7.40 (m, 2H), 7.30 (d, J=3.6 Hz, 1H), 7.24-7.12 (m, 2H), 6.50 (d, J=3.6 Hz, 1H), 5.65 (d, J=10.7 Hz, 1H), 5.49 (d, J=10.7 Hz, 1H), 4.37-4.23 (m, 1H), 3.94-3.80 (m, 1H), 3.62-3.36 (m, 3H), 2.37 (s, 3H), 1.89-1.64 (m, 2H), 1.23 (d, J=6.3 Hz, 3H), 1.01-0.76 (m, 2H), -0.07 (s, 9H).

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 250-mL round-bottom flask purged and maintained with an inert atmosphere of nitrogen, was placed Na (793.5 mg, 34.51 mmol, 6.01 equiv), naphthalene (4.42 g, 34.48 mmol, 6.01 equiv), DME (20 mL). The mixture was stirred at room temperature for 40 min until the formation of Na/naphthalene was complete. Another 250-mL round-bottom flask purged and maintained with an inert atmosphere of nitrogen, was placed 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene (2.8 g, 5.741 mmol, 1 equiv), THE (20 mL). This was followed by the addition of above solution at ˜78 degrees C. The resulting solution was stirred for 2 hr at room temperature. The reaction was then quenched by the addition of 500 mL of NH₄Cl. The resulting solution was extracted with 3×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 1.2 g (62.67%) of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, DMSO-d₆,ppm) δ7.43-7.26 (m, 2H), 6.27 (d, J=3.5 Hz, 1H), 5.43 (d, J=3.4 Hz, 2H), 5.13 (s, 1H), 4.14-4.04 (m, 1H), 3.58-3.39 (m, 3H), 3.28-3.10 (m, 1H), 2.95-2.77 (m, 1H), 2.09-1.85 (m, 1H), 1.85-1.64 (m, 1H), 1.37 (d, J=6.3 Hz, 3H), 0.88-078 (m, 2H),-0.09 (s, 9H).

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene: The crude product (0.5 g) was purified by Chiral-Prep-HPLC with the following conditions: Instrument Name: SHIMADZU LC-20AD, LC parameters: Pump Mode: Binary gradient, Start Conc. of Pump B: 50.0%, Total Flow: 15 mL/min, Phase A: n-Hexane (0.1% DEA), Phase B: Eethanol, Column Name: CHIRALpak IA-3, Length: 50 mm, Internal Diameter: 4.6 mm, Particle Size: 3.0 um, Column Temp: 25° C., PDA Model: SPD-M20A, Wavelength: from 190 nm to 500 nm. This resulted in 220 mg (peak 1 Assumed R) [a]=−6.78° (C=0.129 g/100 mL in CH₂Cl₂, T=27° C.) of (13R or S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil and 230 mg (peak 2 Assumed S) [a]=+11.84° (C=0.106 g/100 mL in CH₂Cl₂,T=27° C.) of (13S or R)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil.

Synthesis of methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate: Into a 40-mL vial purged and maintained with an inert atmosphere of nitrogen, was placed (13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene (210.00 mg, 0.630 mmol, 1.00 equiv), toluene (5.00 mL), methyl 2-bromo-4-(4-[[2-(4-chlorophenyl)-4,4-dimethylcyclohex-1-en-1-yl]methyl]piperazin-1-yl)benzoate (669.87 mg, 1.259 mmol, 2.00 equiv), Cs₂CO₃ (1025.80 mg, 3.148 mmol, 5.00 equiv), Xantphos Pd 2G Precatalyst (111.70 mg, 0.126 mmol, 0.20 equiv). The resulting solution was stirred overnight at 110 degrees C. in an oil bath. The reaction mixture was cooled to room temperature. The resulting mixture was concentrated under vacuum. The residue was applied onto a silica gel column with ethyl acetate/petroleum ether (1:1). This resulted in 240 mg (31.09%) of methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate as yellow oil. LC-MS: (ES, m/z): M+1=784.

Synthesis of 4-(4-[[2-(4-chlorophenyl)-4,4-dimethylcyclohex-1-en-1-yl]methyl]piperazin-1-yl) -2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid: Into a 8-mL round-bottom flask, was placed methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate (200 mg, 0.255 mmol, 1.00 equiv), H₂O (1.00 mL), MeOH (2.00 mL), dioxane (2.00 mL), NaOH (61.18 mg, 1.530 mmol, 6.00 equiv). The resulting solution was stirred for 12 h at 70 degrees C. The resulting mixture was concentrated. The pH value of the solution was adjusted to 5 with HCl (1 mol/L). The solids were collected by filtration. This resulted in 180 mg (91.64%) of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid as a white solid. H-NMR (CDCl₃,300 ppm): δ8.17 (s, 1H), 7.28 (s, 1H), 7.00-6.79 (m, 4H), 6.35 (s, 1H), 5.70-5.67 (d, J=9 Hz, 1H), 5.56-5.52 (d, J=12 Hz, 1H), 4.38-4.35 (m, 1H), 3.81 (s, 1H), 3.58-3.56 (m, 4H), 3.26 (s, 3H), 2.84 (s, 1H), 2.35 (s, 3H), 2.24 (s, 2H), 2.12 (s, 2H), 2.04 (s, 2H), 1.60 (s, 6H), 1.28 (s, 1H), 1.01 (s, 7H), 0.98-0.95 (m, 3H), 0.00 (s, 9H).

Synthesis of 4-(4-[[2-(4-chlorophenyl)-4,4-dimethylcyclohex-1-en1-yl]methyl]iperazin-1-yl)-N-[4-([[(2R)-1,4-dioxan-2-yl]methyl]amino)-3-nitrobenzenesulfonyl]-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide: Into a 8-mL round-bottom flask, was placed 4-([[(2R)-1,4-dioxan-2-yl]methyl]mino)-3-nitrobenzene-1-sulfonamide (41.18 mg, 0.130 mmol, 1 equiv), 4-(4-[[2-(4-chlorophenyl) 4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid (100.00 mg, 0.130 mmol, 1.00 equiv), DCM (3 mL), DMAP (63.42 mg, 0.519 mmol, 4.00 equiv), EDCI (49.76 mg, 0.260 mmol, 2 equiv). The resulting solution was stirred for 12 h at room temperature. The resulting mixture was concentrated. The residue was applied onto a silica gel column with ethyl acetate/petroleum ether (0:1-1:0). This resulted in 80 mg (57.62%) of 4-(4-[[2-(4-chlorophenyl)-4,4-dimethylcyclohex-1-en1-yl]methyl]iperazin-1-yl)-N-[4-([[(2R)-1,4-dioxan-2-yl]methyl]amino)-3-nitrobenzenesulfonyl]-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide as a yellow solid.

### Synthesis of 4-(4-[[2-(4-chlorophenyl)-4,4-dimethylcyclohex-1-en-1-yl]methyl]piperazin-1-yl)-N-[4-([[(2R)-1,4-dioxan-2-yl]methyl]amino)-3-nitrobenzenesulfonyl]-2-[(13R)-13-methyl-14-oxa-2,4,10-triazatricyclo[7.5.0.0\hat{ }[3,7]]tetradeca-1(9),2,5,7-tetraen-10-yl]benzamide: Into a 8-mL round-bottom flask, was placed4-4-[[2-(4-chlorophenyl)4,4-dimethylcyclohex1-en-1-yl]methyl]piperazin-1-yl)-N-[4-([[(2R)-1,4-dioxan-2-yl]methyl]amino)-3-nitrobenzenesulfonyl]-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide (80.00 mg, 0.075 mmol, 1.00 equiv), THE (5.00 mL), ethane-1,2-diamine (89.89 mg, 1.496 mmol, 20 equiv), TBAF (391.05 mg, 1.496 mmol, 20 equiv). The resulting solution was stirred for 48 h at 70 degrees C. The resulting mixture was concentrated. The crude product was purified by Prep-HPLC with the following conditions (Waters-2767): Column, X-bridge RP18, Sum, 19*100 mm; mobile phase, 0.03% ammonia in water (0.03% NH₄HCO₃ & NH₄OH) and CH₃CN (32% CH₃CN up to 52% in 6 min); Detector, UV 254 nm. This resulted in 25 mg (35.58%) of 4-(4-[[2-(4-chlorophenyl)-4,4-dimethylcyclohex-1-en-1-yl]methyl]piperazin-1-yl)-N-[4-([[(2R)-1,4-dioxan-2-yl]methyl]amino)-3-nitrobenzenesulfonyl]-2-[(13R)-13-methyl-14-oxa-2,4,10-triazatricyclo[7.5.0.0\hat{ }[3,7]]tetradeca-1(9),2,5,7-tetraen-10-yl]benzamide as a yellow solid. H-NMR: (d-DMSO, 300M Hz, ppm): δ8.87-8.86 (m, 1H), 8.69 (s, 1H), 8.47 (s, 1H), 8.00-7.92 (m, 2H), 7.13 (s, 1H), 7.00-6.93 (m, 2H), 6.75-6.72 (m, 2H), 6.60-6.50 (s, 1H), 6.19 (s, 1H), 3.92-3.67 (m, 7H), 3.45-3.31 (m, 8H), 2.85 (s, 2H), 2.38-2.25 (m, 6H), 2.09-2.03 (m, 3H), 1.71-1.69 (m, 3H), 1.50-1.46 (m, 2H), 1.06-0.99 (m, 6H)

### Compound 3-34: Synthesis of 4-(4-[[2-(4-chlorophenyl)-4,4-dimethylcyclohex-1-en-1-yl]methyl]piperazin-1-yl)-N-(4- [[(4-fluorooxan-4-yl)methyl]amino]-3-nitrobenzenesulfonyl)-2-[(13S)-13-methyl-14-oxa-2,4,10-triazatricyclo[7.5.0.0\hat{ }[3,7]]tetradeca-1(9),2,5,7-tetraen-10-yl]benzamide

Synthesis of methyl 2-bromo-4-(4-[[2-(4-chlorophenyl)-4,4-dimethylcyclohex-1-en-1-yl]methyl]piperazin-1-yl)benzoate: Into a 250-mL round-bottom flask, was placed a solution of 1-[[2-(4-chlorophenyl)-4,4-dimethylcyclohex-1-en-1-yl]methyl]piperazine (15.09 g, 47.32 mmol, 1.00 equiv) in DMA (150 mL), DIEA (12.9 g, 99.81 mmol, 2.00 equiv), methyl 2-bromo-4-fluorobenzoate (11.6 g, 49.78 mmol, 1.00 equiv). The resulting solution was stirred for 12 h at 100 degree. The reaction mixture was cooled to room temperature. The reaction was then quenched by the addition of 50 mL of water. The resulting solution was extracted with 3×100 mL of ethyl acetate and the organic layers combined. The resulting mixture was washed with 3×100 mL of brine. The mixture was dried over anhydrous sodium sulfate, then filtered and concentrated under vacuum. The residue was applied onto a silica gel column with ethyl acetate/petroleum ether (0:1-1:5). This resulted in 7 g (crude) of methyl 2-bromo-4-(4-[[2-(4-chlorophenyl)-4,4-dimethylcyclohex-1-en-1-yl]methyl]piperazin-1-yl)benzoate as yellow oil. LC-MS: (ES, m/z): M+1=533, 531.

### Synthesis of 1,6-dioxaspiro[2.5]octane-2-carbonitrile: Into a 3000-mL round-bottom flask, was placed oxan-4-one (204.2 g, 2039.74 mmol, 1.1 equiv), 2-chloroacetonitrile (140 g, 1854.30 mmol, 1 equiv), t-BuOH (200 mL). The resulting solution was stirred for 30 min at 25 degrees C. This was followed by the addition of a solution of t-BuOK (249.7 g, 2225.25 mmol, 1.20 equiv) in tert-Butanol (2000 mL) dropwise with stirring at 25 degrees C. in 40 min. The resulting solution was stirred for overnight at 25 degrees C. The resulting solution was diluted with 400 mL of water and quenched with 80 mL of 10% hydrogen chloride. The resulting mixture was concentrated to one-third of its volume. The resulting solution was extracted with 3×800 mL of ether and the organic layers combined. The resulting mixture was washed with 3000 mL of brine. The mixture was dried over anhydrous sodium sulfate and concentrated under vacuum. This resulted in 162 g (62.78%) of 1,6-dioxaspiro[2.5]octane-2-carbonitrile as brown oil. ¹H NMR (300 MHz, CDCl₃-d,ppm) δ3.95-3.81 (m, 4H), 2.17-2.02 (m, 1H), 1.99-1.77 (m, 2H), 1.66-1.54 (m, 1H)

Synthesis of 2-(4-fluorooxan-4-yl)-2-hydroxyacetonitrile: Into a 2000-mL round-bottom flask, was placed 1,6-dioxaspiro[2.5]octane-2-carbonitrile (162 g, 1164.18 mmol, 1 equiv), DCM (800 mL). This was followed by the addition of 70% HF/Py (150 mL, 0.95 equiv) dropwise with stirring at 0 degrees C. The resulting solution was stirred for overnight at 40 degrees C. in an oil bath. The resulting solution was diluted with 1500 mL of ethyl acetate and poured into Sat. aqueous NaHCO₃. Additional solid NaHCO₃was used to neutralize the mixture carefully until bubbling ceased. The resulting solution was extracted with 2×1000 mL of ethyl acetate and the organic layers combined. The resulting mixture was washed with 1×3000 mL of 1% hydrogen chloride and 1×3000 mL of brine. The mixture was dried over anhydrous sodium sulfate and concentrated under vacuum. The residue was applied onto a silica gel column with ethyl acetate/petroleum ether (1:2). This resulted in 95 g (51.27%) of 2-(4-fluorooxan-4-yl)-2-hydroxyacetonitrile as a yellow solid. ¹H NMR (300 MHz, CDCl₃-d,ppm) δ4.39 (d, J=15.6 Hz, 1H), 4.05-3.90 (m, 2H), 3.83-3.70 (m, 2H), 2.08-1.77 (m, 4H).

Synthesis of (4-fluorooxan-4-yl)methanol: Into a 2000-mL round-bottom flask, was placed 2-(4-fluorooxan-4-yl)-2-hydroxyacetonitrile (95 g, 596.88 mmol, 1 equiv), i-PrOH (1500 mL), H₂O (375 mL). This was followed by the addition of NaBH₄ (35 g, 925.12 mmol, 1.55 equiv), in portions at 0 degrees C. The resulting solution was stirred for 2 hr at 25 degrees C. The reaction was then quenched by the addition of 100 mL of acetone and stirred for another 1 h. The solids were filtered out. EtOAc (200 mL) was used to wash the solid. The filtration was concentrated under vacuum. The residue was applied onto a silica gel column with ethyl acetate/petroleum ether (1:1). This resulted in 66 g (82.43%) of (4-fluorooxan-4-yl)methanol as yellow oil. ¹H NMR (300 MHz, CDCl₃-d,ppm) δ3.90-3.70 (m, 2H), 3.62 (d, J=20.6 Hz, 1H), 1.88-1.61 (m, 2H).

Synthesis of (4-fluorooxan-4-yl)methyl methanesulfonate: Into a 2000-mL round-bottom flask, was placed (4-fluorooxan-4-yl)methanol (66 g, 491.99 mmol, 1 equiv), DCM (600 mL), TEA (74.7 g, 737.98 mmol, 1.5 equiv). This was followed by the addition of MsCl (84.5 g, 737.66 mmol, 1.50 equiv) dropwise with stirring at 0 degrees C. The resulting solution was stirred for 2 hr at 25 degrees C. The resulting solution was diluted with 2000 mL of water. The resulting solution was extracted with 2×500 mL of dichloromethane. The combined organic layer was dried over anhydrous sodium sulfate and concentrated under vacuum. This resulted in 100 g (95.77%) of (4-fluorooxan-4-yl)methyl methanesulfonate as yellow oil. ¹H NMR (300 MHz, DMSO) δ 4.32 (d, J=21.6 Hz, 1H), 3.83-3.49 (m, 2H), 1.92-1.62 (m, 2H).

Synthesis of 4-(azidomethyl)-4-fluorooxane: Into a 2000-mL 4-necked round-bottom flask, was placed (4-fluorooxan-4-yl)methyl methanesulfonate (100 g, 471.16 mmol, 1 equiv), DMF (1000 mL), NaHCO₃(93.0 g, 1107.06 mmol, 2.35 equiv), NaN₃ (90 g, 1384.40 mmol, 2.94 equiv). The resulting solution was stirred for overnight at 120 degrees C. in an oil bath. The reaction mixture was cooled to room temperature. The reaction was then quenched by the addition of 3000 mL of water. The resulting solution was extracted with 3×500 mL of ethyl acetate and the organic layers combined. The resulting mixture was washed with 3×3000 mL of brine. The mixture was dried over anhydrous sodium sulfate and concentrated under vacuum. This resulted in 70 g (93.34%) of 4-(azidomethyl)-4-fluorooxane as yellow oil without any further purification.

Synthesis of 1-(4-fluorooxan-4-yl)methanamine: Into a 2000-mL pressure tank reactor, was placed 4-(azidomethyl)-4-fluorooxane (70 g, 439.80 mmol, 1 equiv), EtOAc (700 mL), 10% Pd/C (7 g, 3472.43 mmol). The flask was evacuated and flushed three times with nitrogen, followed by flushing with hydrogen(2-5 atm pressure). The resulting solution was stirred for overnight at 25 degrees C. The solids were filtered out. The resulting mixture was concentrated under vacuum. This resulted in 52 g (crude) of 1-(4-fluorooxan-4-yl)methanamine as yellow oil. ¹H NMR (300 MHz, MeOD-d4,ppm) δ3.88-3.69 (m, 2H), 2.76 (d, J=20.5 Hz, 1H), 1.85-1.68 (m, 2H).

### Synthesis of 4-[[(4-fluorooxan-4-yl)methyl]amino]-3-nitrobenzene-1-sulfonamide: Into a 2000-mL round-bottom flask, was placed 1-(4-fluorooxan-4-yl)methanamine (52.0 g, 390.49 mmol, 1.00 equiv), THE (1000 mL), 4-fluoro-3-nitrobenzene-1-sulfonamide (86 g, 390.61 mmol, 1 equiv), Cs₂CO₃ (254.5 g, 781.21 mmol, 2 equiv). The resulting solution was stirred for 4 hr at 50 degrees C. in an oil bath. The resulting solution was diluted with 3000 mL of water. The solids were collected by filtration. This resulted in 50.6 g (38.86%) of 4-[[(4-fluorooxan-4-yl)methyl]amino]-3-nitrobenzene-1-sulfonamide as a yellow solid, and some crude product from aqueous phase. ¹H NMR (300 MHz, DMSO-d₆,ppm) δ8.58 (t, J=6.4 Hz, 1H), 8.48 (d, J=2.2 Hz, 1H), 7.83 (dd, J=9.1, 2.0 Hz, 1H), 7.41 (d, J=9.2 Hz, 1H), 7.34 (s, 2H), 3.84-3.70 (m, 4H), 3.59-3.45 (m, 2H), 1.87-1.66 (m, 4H)

Synthesis of N-(3-hydroxybutyl)-4-methylbenzene-1-sulfonamide: Into a 250-mL round-bottom flask purged and maintained with an inert atmosphere of nitrogen, was placed 4-aminobutan-2-ol hydrochloride (4 g, 31.847 mmol, 1 equiv), DCM (40 mL), TEA (3.56 g, 35.181 mmol, 1.10 equiv). This was followed by the addition of 4-methylbenzene-1-sulfonyl chloride (6.08 g, 31.893 mmol, 1.00 equiv) in portions at 0 degrees C. The resulting solution was stirred for 2 h at 25 degrees C. The resulting mixture was concentrated under vacuum. The residue was applied onto a silica gel column with ethyl acetate/petroleum ether (1:1). This resulted in 4.4 g (56.78%) of N-(3-hydroxybutyl)-4-methylbenzene-1-sulfonamide as colorless oil. ¹H NMR (300 MHz, CDCl₃-d,ppm) δ7.82-7.73 (d, J=9.0 Hz, 2H), 7.39-7.30 (d, J=9.0 Hz, 2H), 4.06-3.82 (m, 1H), 3.24-3.16 (m, 1H), 3.06-3.00 (m, 1H), 2.45 (s, 3H), 1.70-1.55 (m, 2H), 1.20 (d, J=6.2 Hz, 3H).

Synthesis of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide: Into a 250-mL 3-necked round-bottom flask purged and maintained with an inert atmosphere of nitrogen, was placed N-(3-hydroxybutyl)-4-methylbenzene-1-sulfonamide (4.4 g, 18.083 mmol, 1.10 equiv), THE (60 mL). This was followed by the addition of NaH (1.97 g, 49.255 mmol, 3.00 equiv, 60%), in portions at 0 degrees C. The resulting solution was stirred for 0.5 h at 0 degrees C. To this was added 5-bromo-6-fluoro-1-[[2-(trimethylsilyl)ethoxy]methyl]-1H-pyrrolo[2,3-b]pyridine (5.66 g, 16.392 mmol, 1 equiv). The resulting solution was stirred overnight at 50 degrees C. in an oil bath. The reaction mixture was cooled to 25 degree C. The reaction was then quenched by the addition of 500 mL of NH₄Cl. The resulting solution was extracted with 2×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 6 g (64.37%) of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide as colorless oil. ¹H NMR (300 MHz, CDCl₃-d,ppm) δ8.04 (s, 1H), 7.74-7.63 (m, 2H), 7.23-7.11 (m, 3H), 6.41 (d, J=3.6 Hz, 1H), 5.56 (d, J=3.3 Hz, 2H), 5.89-5.33 (m, 1H), 3.59-3.46 (m, 2H), 3.17 (t, J=6.0 Hz, 2H), 2.37 (s, 3H), 2.08-1.78 (m, 2H), 1.37 (d, J=6.2 Hz, 3H), 0.94-0.85 (m, 2H), -0.06 (s, 9H).

Synthesis of 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 250-mL round-bottom flask purged and maintained with an inert atmosphere of nitrogen, was placed N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide (6 g, 10.552 mmol, 1 equiv), DMSO (60 mL), pyridine-2-carboxylic acid (1.04 g, 8.448 mmol, 0.80 equiv), CuI (2.41 g, 12.654 mmol, 1.20 equiv), K₂CO₃ (4.38 g, 31.692 mmol, 3.00 equiv).

The resulting solution was stirred for 2 days at 125 degrees C. in an oil bath. The reaction mixture was cooled to 25 degree C. The resulting solution was diluted with 500 mL of water. The resulting solution was extracted with 3×200 mL of ethyl acetate and the organic layers combined. The resulting mixture was washed with 2×1000 ml of brine. The mixture was dried over anhydrous sodium sulfate and concentrated under vacuum. The residue was applied onto a silica gel column with ethyl acetate/petroleum ether (1:3). This resulted in 2.8 g (54.41%) of 13-methyl-10-(4-methylbenzenesulfonyl)-4-[[2-(trimethylsilyl) ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, CDCl₃-d,ppm) δ8.10 (s, 1H), 7.53-7.40 (m, 2H), 7.30 (d, J=3.6 Hz, 1H), 7.24-7.12 (m, 2H), 6.50 (d, J=3.6 Hz, 1H), 5.65 (d, J=10.7 Hz, 1H), 5.49 (d, J=10.7 Hz, 1H), 4.37-4.23 (m, 1H), 3.94-3.80 (m, 1H), 3.62-3.36 (m, 3H), 2.37 (s, 3H), 1.89-1.64 (m, 2H), 1.23 (d, J=6.3 Hz, 3H), 1.01-0.76 (m, 2H), -0.07 (s, 9H).

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 250-mL round-bottom flask purged and maintained with an inert atmosphere of nitrogen, was placed Na (793.5 mg, 34.51 mmol, 6.01 equiv), naphthalene (4.42 g, 34.48 mmol, 6.01 equiv), DME (20 mL). The mixture was stirred at room temperature for 40 min until the formation of Na/naphthalene was complete. Another 250-mL round-bottom flask purged and maintained with an inert atmosphere of nitrogen, was placed 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene (2.8 g, 5.741 mmol, 1 equiv), THE (20 mL). This was followed by the addition of above solution at ˜78 degrees C. The resulting solution was stirred for 2 hr at room temperature. The reaction was then quenched by the addition of 500 mL of NH₄Cl. The resulting solution was extracted with 3×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 1.2 g (62.67%) of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, DMSO-d₆,ppm) δ7.43-7.26 (m, 2H), 6.27 (d, J=3.5 Hz, 1H), 5.43 (d, J=3.4 Hz, 2H), 5.13 (s, 1H), 4.14-4.04 (m, 1H), 3.58-3.39 (m, 3H), 3.28-3.10 (m, 1H), 2.95-2.77 (m, 1H), 2.09-1.85 (m, 1H), 1.85-1.64 (m, 1H), 1.37 (d, J=6.3 Hz, 3H), 0.88-078 (m, 2H),-0.09 (s, 9H).

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene: The crude product (0.5 g) was purified by Chiral-Prep-HPLC with the following conditions: Instrument Name: SHIMADZU LC-20AD, LC parameters: Pump Mode: Binary gradient, Start Conc. of Pump B: 50.0%, Total Flow: 15 mL/min, Phase A: n-Hexane (0.1% DEA), Phase B: Eethanol, Column Name: CHIRALpak IA-3, Length: 50 mm, Internal Diameter: 4.6 mm, Particle Size: 3.0 um, Column Temp: 25° C., PDA Model: SPD-M20A, Wavelength: from 190 nm to 500 nm. This resulted in 220 mg (peak 1 Assumed R) [a]=−6.78° (C=0.129 g/100 mL in CH₂Cl₂, T=27° C.) of (13R or S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil and 230 mg (peak 2 Assumed S) [a]=+11.84° (C=0.106 g/100 mL in CH₂Cl₂, T=27° C.) of (13S or R)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil.

### Synthesis of methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate: Into a 40-mL vial purged and maintained with an inert atmosphere of nitrogen, was placed (13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene (210.00 mg, 0.630 mmol, 1.00 equiv), toluene (5.00 mL), methyl 2-bromo-4-(4-[[2-(4-chlorophenyl)-4,4-dimethylcyclohex-1-en-1-yl]methyl]piperazin-1-yl)benzoate (669.87 mg, 1.259 mmol, 2.00 equiv), Cs₂CO₃ (1025.80 mg, 3.148 mmol, 5.00 equiv), Xantphos Pd 2G Precatalyst (111.70 mg, 0.126 mmol, 0.20 equiv). The resulting solution was stirred overnight at 110 degrees C. in an oil bath. The reaction mixture was cooled to room temperature. The resulting mixture was concentrated under vacuum. The residue was applied onto a silica gel column with ethyl acetate/petroleum ether (1:1). This resulted in 240 mg (31.09%) of methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate as yellow oil.LC-MS: (ES, m/z): M+1=784

### Synthesis of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl]benzoic acid: Into a 8-mL round-bottom flask, was placed methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate (200 mg, 0.255 mmol, 1.00 equiv), H₂O (1.00 mL), MeOH (2.00 mL), dioxane (2.00 mL), NaOH (61.18 mg, 1.530 mmol, 6.00 equiv). The resulting solution was stirred for 12 h at 70 degrees C. The resulting mixture was concentrated. The pH value of the solution was adjusted to 5 with HCl (1 mol/L). The solids were collected by filtration. This resulted in 180 mg (91.64%) of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid as a white solid. H-NMR: (CDCl₃, 300 ppm): δ8.17 (s, 1H), 7.28 (s, 1H), 7.00-6.79 (m, 4H), 6.35 (s, 1H), 5.70-5.67 (d, J=9 Hz, 1H), 5.56-5.52 (d, J=12 Hz, 1H), 4.38-4.35 (m, 1H), 3.81 (s, 1H), 3.58-3.56 (m, 4H), 3.26 (s, 3H), 2.84 (s, 1H), 2.35 (s, 3H), 2.24 (s, 2H), 2.12 (s, 2H), 2.04 (s, 2H), 1.60 (s, 6H), 1.28 (s, 1H), 1.01 (s, 7H), 0.98-0.95 (m, 3H), 0.00 (s, 9H)

Synthesis of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide: Into a 8-mL round-bottom flask, was placed 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid (100.00 mg, 0.130 mmol, 1.00 equiv), 4-[[(4-fluorooxan-4-yl)methyl]amino]-3-nitrobenzene-1-sulfonamide (43.26 mg, 0.130 mmol, 1.00 equiv), DCM (3 mL), DMAP (63.42 mg, 0.519 mmol, 4.00 equiv), EDCI (49.76 mg, 0.260 mmol, 2.00 equiv). The resulting solution was stirred for 12 h at room temperature. The resulting mixture was concentrated. The residue was applied onto a silica gel column with ethyl acetate/petroleum ether (0:1-1:0). This resulted in 80 mg (56.77%) of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzamide as a yellow solid.

Synthesis of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(13S)-13-methyl-14-oxa-2,4,10-triazatricyclo[7.5.0.0\hat{ }[3,7]]tetradeca-1(9),2,5,7-tetraen-10-yl]benzamide: Into a 8-mL round-bottom flask, was placed 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(13S)-13-methyl-4-[[2-(trimethylsilyl)ethoxy]methyl]-14-oxa-2,4,10-triazatricyclo[7.5.0.0 \hat{ }[3,7]]tetradeca-1(9),2,5,7-tetraen-10-yl]benzamide (80.00 mg, 0.074 mmol, 1.00 equiv), THE (5 mL), TBAF (385.28 mg, 1.474 mmol, 20 equiv), ethane-1,2-diamine (88.56 mg, 1.474 mmol, 20.00 equiv). The resulting solution was stirred for 48 h at 70 degrees C. The resulting mixture was concentrated. The crude product was purified by Prep-HPLC with the following conditions (Waters-2767): Column, X-bridge RP18, Sum, 19*100 mm; mobile phase, 0.03% ammonia in water (0.03% NH₄HCO₃ & NH₄OH) and CH₃CN (32% CH₃CN up to 52% in 6 min); Detector, UV 254 nm. This resulted in 25 mg (35.51%) of 4-(4-[[2-(4-chlorophenyl)-4,4-dimethylcyclohex-1-en-1-yl]methyl]piperazin-1-yl)-N-(4-[[(4-fluorooxan-4-yl)methyl]amino]-3-nitrobenzenesulfonyl)-2-[(13S)-13-methyl-14-oxa-2,4,10-triazatricyclo[7.5.0.0\hat{ }[3,7]]tetradeca-1(9),2,5,7-tetraen-10-yl]benzamide as a yellow solid. H-NMR: (d-DMSO, 300M Hz, ppm): δ8.59-8.67 (m, 1H), 8.69 (s, 1H), 8.48 (s, 1H), 8.03-7.94 (m, 2H), 7.15-7.14 (m, 2H), 6.97-6.93 (m, 3H), 6.76-6.73 (m, 3H), 6.20 (s, 1H), 3.96-3.69 (m, 5H), 3.49-3.25 (m, 7H), 2.85 (s, 2H), 2.37-2.25 (m, 6H), 2.15-2.04 (m, 3H), 1.89-1.83 (m, 3H), 1.71-1.69 (m, 4H), 1.50-1.46 (m, 2H), 1.06-0.99 (m, 6H).

### Compound 3-35: Synthesis of 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]-N-[3-nitro-4-[(oxan-4-ylmethyl)amino]benzenesulfonyl]benzamide

Synthesis of ethyl 2-cyano-3-hydroxy-2-methylpropanoate: Into a 500-mL round-bottom flask, was placed ethyl 2-cyanopropionate (20.00 g, 157.303 mmol, 1.00 equiv), CH₃CN (200.00 mL), formaldehyde (32.00 g, 394.325 mmol, 2.51 equiv, 37%), TEA (800.00 mg, 7.906 mmol, 0.05 equiv). The resulting solution was stirred overnight at 50 degrees C. in an oil bath. The reaction mixture was cooled to room temperature. The resulting mixture was concentrated under vacuum. The residue was applied onto a silica gel column and eluted with ethyl acetate/petroleum ether (1:4). This resulted in 20 g (80.90%) of ethyl 2-cyano-3-hydroxy-2-methylpropanoate as colorless oil. ¹H NMR (300 MHz, CDCl₃-d,ppm) δ5.01-4.72 (m, 1H), 4.38-4.23 (m, 2H), 4.04-3.82 (m, 2H), 1.60 (d, J=2.6 Hz, 3H), 1.35 (td, J=7.1, 2.7 Hz, 3H).

Synthesis of 3-hydroxy-2-(hydroxymethyl)-2-methylpropanenitrile: Into a 250-mL round-bottom flask, was placed ethyl 2-cyano-3-hydroxy-2-methylpropanoate (12.00 g, 76.351 mmol, 1.00 equiv), MeOH (120.00 mL). This was followed by the addition of NaBH₄ (4.40 g, 116.301 mmol, 1.52 equiv), in portions at 0 degrees C. The resulting solution was stirred for 1.5 hr at 25 degrees C. The reaction was then quenched by the addition of 20 mL of water. The resulting mixture was concentrated under vacuum. The residue was applied onto a silica gel column and eluted with dichloromethane/methanol (10:1). This resulted in 8 g (91.01%) of 3-hydroxy-2-(hydroxymethyl)-2-methylpropanenitrile as light yellow oil. ¹H NMR (300 MHz, MeOD-d4,ppm) δ3.74-3.50 (m, 4H), 1.29 (s, 3H).

### Synthesis of 2-[[(5-bromo-1-[[2-(trimethylsilyl)ethoxy]methyl]pyrrolo[2,3-b]pyridin-6-yl)oxy]methyl]-3-hydroxy-2-methylpropanenitrile: Into a 500-mL round-bottom flask, was placed 5-bromo-6-fluoro-1-[[2-(trimethylsilyl)ethoxy]methyl]pyrrolo[2,3-b]pyridine (12.00 g, 34.754 mmol, 1.00 equiv), CH₃CN (200.00 mL), 3-hydroxy-2-(hydroxymethyl)-2-methylpropanenitrile (8.00 g, 69.485 mmol, 2.00 equiv), Cs₂CO₃ (28.34 g, 86.981 mmol, 2.50 equiv). The resulting solution was stirred for 12 hr at 80 degrees C. in an oil bath. The reaction mixture was cooled to room temperature. The resulting solution was diluted with 500 mL of H₂O. The resulting solution was extracted with 2×300 mL of ethyl acetate and the organic layers combined. The resulting mixture was washed with 1 x1000 ml of brine. The mixture was dried over anhydrous sodium sulfate. The solids were filtered out. The resulting mixture was concentrated under vacuum. The residue was applied onto a silica gel column and eluted with ethyl acetate/petroleum ether (1:5). This resulted in 6 g (39.20%) of 2-[[(5-bromo-1-[[2-(trimethylsilyl)ethoxy]methyl]pyrrolo[2,3-b]pyridin-6-yl)oxy]methyl]-3-hydroxy-2-methylpropanenitrile as light yellow oil. ¹H NMR (300 MHz, CDCl₃-d,ppm) δ8.08 (s, 1H), 7.18 (d, J=3.6 Hz, 1H), 6.42 (d, J=3.6 Hz, 1H), 5.55 (s, 2H), 4.70 (d, J=10.8 Hz, 1H), 4.53 (d, J=10.9 Hz, 1H), 3.85 (d, J=4.0 Hz, 2H), 3.62-3.46 (m, 2H), 1.53 (s, 3H), 1.05-0.85 (m, 2H),-0.03 (s, 8H)

Synthesis of N-[6-[2-cyano-2-(hydroxymethyl)-2-methylethoxy]-1-[[2-(trimethylsilyl)ethoxy]methyl]pyrrolo[2,3-b]pyridin-5-yl]-4-methylbenzenesulfonamide: Into a 250-mL round-bottom flask purged and maintained with an inert atmosphere of nitrogen, was placed 2-[[(5-bromo-1-[[2-(trimethylsilyl)ethoxy]methyl]pyrrolo[2,3-b]pyridin-6-yl)oxy]methyl]-3-hydroxy-2-methylpropanenitrile (6.00 g, 13.624 mmol, 1.00 equiv), DMSO (50.00 mL), p-toluenesulfonamide (4.67 g, 27.276 mmol, 2.00 equiv), picolinic acid (1.35 g, 10.966 mmol, 0.80 equiv), CuI (3.12 g, 16.382 mmol, 1.20 equiv), K2CO₃ (5.66 g, 40.954 mmol, 3.01 equiv). The resulting solution was stirred for 2 days at 120 degrees C. in an oil bath. The reaction mixture was cooled to room temperature. The resulting solution was diluted with 500 mL of H₂O. The resulting solution was extracted with 3×200 mL of ethyl acetate and the organic layers combined. The resulting mixture was washed with 2 x1000 ml of brine.

The mixture was dried over anhydrous sodium sulfate and concentrated under vacuum. The residue was applied onto a silica gel column and eluted with ethyl acetate/petroleum ether (1:1). This resulted in 2.45 g (33.89%) of N-[6-[2-cyano-2-(hydroxymethyl)-2-methylethoxy]-1-[[2-(trimethylsilyl)ethoxy]methyl]pyrrolo[2,3-b]pyridin-5-yl]-4-methylbenzenesulfonamide as a white solid. ¹H NMR (300 MHz, CDCl₃-d,ppm) δ8.14 (s, 1H), 7.68-7.58 (m, 2H), 7.25-7.13 (m, 3H), 6.76 (s, 1H), 6.48 (d, J=3.6 Hz, 1H), 5.47 (d, J=4.4 Hz, 2H), 4.41 (d, J=11.2 Hz, 1H), 4.29 (d, J=11.3 Hz, 1H), 3.61 (dd, J=7.9, 6.3 Hz, 1H), 3.56-3.43 (m, 4H), 2.38 (s, 3H), 1.34 (s, 3H), 0.97-0.84 (m, 2H),-0.05 (s, 9H).

Synthesis of 12-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene-12-carbonitrile: Into a 250-mL round-bottom flask purged and maintained with an inert atmosphere of nitrogen, was placed N-[6-[2-cyano-2-(hydroxymethyl)-2-methylethoxy]-1-[[2-(trimethylsilyl)ethoxy]methyl]pyrrolo[2,3-b]pyridin-5-yl]-4-methylbenzenesulfonamide (2.45 g, 4.616 mmol, 1.00 equiv), THE (30 mL), PPh₃ (2.42 g, 9.227 mmol, 2.00 equiv). This was followed by the addition of DEAD (1.61 g, 9.245 mmol, 2.00 equiv) dropwise with stirring at 0 degrees C. The resulting solution was stirred for 1 hr at 25 degrees C. The resulting solution was diluted with 500 mL of H₂O. The resulting solution was extracted with 2×200 mL of ethyl acetate and the organic layers combined. The resulting mixture was washed with 1×500 ml of brine. The mixture was dried over anhydrous sodium sulfate and concentrated under vacuum. The residue was applied onto a silica gel column and eluted with ethyl acetate/petroleum ether (1:3). This resulted in 2 g (84.50%) of 12-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene-12-carbonitrile as a yellow solid.

Synthesis of 12-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene-12-carbonitrile: Into a 40-mL vial purged and maintained with an inert atmosphere of nitrogen, was placed Na (539.00 mg, 23.445 mmol, 6.01 equiv), Naphthalene (3.00 g, 23.406 mmol, 6.00 equiv), DME (10.00 mL). The mixture was stirred at room temperature for 40 min until the formation of Na/naphthalene was complete. This was followed by the addition of above solution to the 12-methyl-10-(4-methylbenzenesulfonyl)-4-[[2-(trimethylsilyl)ethoxy]methyl]-14-oxa-2,4,10-triazatricyclo[7.5.0.0 \hat{ }[3,7]]tetradeca-1(9),2,5,7-tetraene-12-carbonitrile (2.00 g, 3.901 mmol, 1.00 equiv) in THE (10.00 mL) at ˜78 degrees C. The resulting solution was stirred for 2 hr at 25 degrees C. The reaction was then quenched by the addition of 500 mL of aqueous NH₄Cl. The resulting solution was extracted with 3×200 mL of ethyl acetate and the organic layer was combined. The resulting mixture was washed with 1×500 ml of brine. The mixture was dried over anhydrous sodium sulfate and concentrated under vacuum. The residue was applied onto a silica gel column and eluted with ethyl acetate/petroleum ether (1:3). This resulted in 1 g (71.50%) of 12-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene-12-carbonitrile as yellow oil. ¹H NMR (300 MHz, DMSO-d_{6, ppm}) δ7.44 (s, 1H), 7.40 (d, J=3.5 Hz, 1H), 6.34 (d, J=3.5 Hz, 1H), 5.65-5.52 (m, 1H), 5.44 (s, 2H), 4.51-4.36 (m, 1H), 3.86 (d, J=12.2 Hz, 1H), 3.56-3.42 (m, 3H), 2.94 (dd, J=13.2, 2.3 Hz, 1H), 1.31 (s, 3H), 0.89-0.71 (m, 2H),-0.08 (s, 9H).

Synthesis of methyl 4-(4-[[2-(4-chlorophenyl)-4,4-dimethylcyclohex-1-en-1-yl]methyl]piperazin-1-yl)-2-(12-cyano-12-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 100-mL round-bottom flask purged and maintained with an inert atmosphere of nitrogen, was placed methyl 2-bromo-4-(4-[[2-(4-chlorophenyl)-4,4-dimethylcyclohex-1-en-1-yl]methyl]piperazin-1-yl)benzoate (1.18 g, 2.218 mmol, 1.99 equiv), toluene (10.00 mL), 12-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene-12-carbonitrile (400.00 mg, 1.116 mmol, 1.00 equiv), Cs₂CO₃ (1.82 g, 5.586 mmol, 5.01 equiv), Xantphos Pd 2G Precatalyst (149.00 mg, 0.168 mmol, 0.15 equiv). The resulting solution was stirred 2 days at 110 degrees C. in an oil bath. The reaction mixture was cooled to room temperature. The resulting mixture was concentrated under vacuum. The residue was applied onto a silica gel column and eluted with ethyl acetate/petroleum ether (1:1). This resulted in 330 mg (36.54%) of methyl 4-(4-[[2-(4-chlorophenyl)-4,4-dimethylcyclohex-1-en-1-yl]methyl]piperazin-1-yl)-2-(12-cyano-12-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as yellow oil. LC-MS: (ES, m/z): M+1=809.

Synthesis of methyl 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]benzoate: Into a 100-mL round-bottom flask, was placed methyl 4-(4-[[2-(4-chlorophenyl)-4,4-dimethylcyclohex-1-en-1-yl]methyl]piperazin-1-yl)-2-(12-cyano-12-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (330.00 mg, 0.408 mmol, 1.00 equiv), THE (20.00 mL), TBAF.3H₂O (5.00 g), ethylenediamine (2.50 g, 41.597 mmol, 102.04 equiv). The resulting solution was stirred for 2 days at 70 degrees C. in an oil bath. The reaction mixture was cooled to room temperature. The resulting mixture was concentrated under vacuum. The residue was applied onto a silica gel column with ethyl acetate/petroleum ether (2:1). This resulted in 220 mg (79.45%) of methyl 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]benzoate as a light yellow solid. LC-MS: (ES, m/z): M+1=679.

### Synthesis of 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]benzoic acid Into a 40-mL vial, was placed methyl 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]benzoate (190.00 mg, 0.280 mmol, 1.00 equiv), MeOH (12.00 mL), dioxane (12.00 mL), H₂O (4.00 mL), LiOH.H₂O (353.00 mg, 8.412 mmol, 30.07 equiv). The resulting solution was stirred overnight at 60 degrees C. in an oil bath. The reaction mixture was cooled to room temperature. The resulting mixture was concentrated under vacuum. The pH value of the solution was adjusted to 6-7 with HCl (2 mol/L). The resulting solution was extracted with 2×100 mL of ethyl acetate and the organic layers combined. The resulting mixture was washed with 1×500 ml of brine. The mixture was dried over anhydrous sodium sulfate and concentrated under vacuum. The residue was purified by Prep-TLC with ethyl acetate. This resulted in 80 mg (42.99%) of 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]benzoic acid as a light yellow solid. LC-MS: (ES, m/z): M+1=665

Synthesis of 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]-N-[3-nitro-4-[(oxan-4-ylmethyl)amino]benzenesulfonyl]benzamide: Into a 40-mL vial, was placed 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]benzoic acid (40.00 mg, 0.060 mmol, 1.00 equiv), DCM (2.00 mL), 3-nitro-4-[(oxan-4-ylmethyl)amino]benzenesulfonamide (19.00 mg, 0.060 mmol, 1.00 equiv), EDCI (23.00 mg, 0.120 mmol, 2.00 equiv), DMAP (29.00 mg, 0.237 mmol, 3.95 equiv). The resulting solution was stirred overnight at 25 degrees C. The resulting mixture was concentrated under vacuum. The crude product was purified by Prep-HPLC with the following conditions (2 #SHIMADZU (HPLC-01)): Column, XBridge Prep C18 OBD Column, 5 um,19*150 mm; mobile phase, ACN and Water (0.05% NH₃.H₂O) (20% Phase B up to 75% in 1 min, up to 95% in 7 min, hold 95% in 1 min, down to 20% in 1 min); Detector, 254/220 nm. This resulted in 18.8 mg (32.48%) of 4-(4-[[2-(4-chlorophenyl)-4,4-dimethylcyclohex-1-en-1-yl]methyl]piperazin-1-yl)-2-[12-cyano-12-methyl-14-oxa-2,4,10-triazatricyclo[7.5.0.0\hat{ }[3,7]]tetradeca-1(9),2,5,7-tetraen-10-yl]-N-[3-nitro-4-[(oxan-4-ylmethyl)amino]benzenesulfonyl]benzamide as a yellow solid.LC-MS: (ES, m/z): M+1=962, R.T=1.461 min. The measurements of the retention were done with a reversed phase column (C18). Shimadzu LCMS 2020; SUPELCO Ascentis Sxpress C18, 50*3.0 mm, 2.7 microm; Eluent A: water (0.05% TFA); Eluent B: Acetonitrile; linear gradient from 5% acetonitrile to 100% acetonitrile in 3.0 minutes; Oven temperature 40° C.; flow: 1.5 mL/min. H NMR (300 MHz, DMSO-d_{6, ppm}) δ8.55 (s, 1H), 8.34 (s, 1H), 7.60-7.31 (m, 4H), 7.23-7.00 (m, 3H), 6.76 (d, J=26.1 Hz, 4H), 6.04 (s, 1H), 3.88 (dd, J=11.7, 4.0 Hz, 2H), 3.38-3.12 (m, 7H), 2.77 (d, J=19.8 Hz, 2H), 2.24 (d, J=20.9 Hz, 6H), 1.99 (s, 4H), 1.66 (d, J=12.4 Hz, 2H), 1.42 (t, J=6.3 Hz, 2H), 1.36-1.03 (m, 8H), 0.96 (s, 6H).

### Compound 3-36: Synthesis of 4-(4-[[2-(4-chlorophenyl)-4,4-dimethylcyclohex-1-en-1-yl]methyl]piperazin-1-yl)-2-[(13S)-13-methyl-14-oxa-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide

Synthesis of N-(3-hydroxybutyl)-4-methylbenzene-1-sulfonamide: Into a 250-mL round-bottom flask purged and maintained with an inert atmosphere of nitrogen, was placed 4-aminobutan-2-ol hydrochloride (4 g, 31.847 mmol, 1 equiv), DCM (40 mL), TEA (3.56 g, 35.181 mmol, 1.10 equiv). This was followed by the addition of 4-methylbenzene-1-sulfonyl chloride (6.08 g, 31.893 mmol, 1.00 equiv) in portions at 0 degrees C. The resulting solution was stirred for 2 h at 25 degrees C. The resulting mixture was concentrated under vacuum. The residue was applied onto a silica gel column with ethyl acetate/petroleum ether (1:1). This resulted in 4.4 g (56.78%) of N-(3-hydroxybutyl)-4-methylbenzene-1-sulfonamide as colorless oil. 1H NMR (300 MHz, CDCl₃-d,ppm) δ7.82-7.73 (d, J=9.0 Hz, 2H), 7.39-7.30 (d, J=9.0 Hz, 2H), 4.06-3.82 (m, 1H), 3.24-3.16 (m, 1H), 3.06-3.00 (m, 1H), 2.45 (s, 3H), 1.70-1.55 (m, 2H), 1.20 (d, J=6.2 Hz, 3H).

Synthesis of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide: Into a 250-mL 3-necked round-bottom flask purged and maintained with an inert atmosphere of nitrogen, was placed N-(3-hydroxybutyl)-4-methylbenzene-1-sulfonamide (4.4 g, 18.083 mmol, 1.10 equiv), THE (60 mL). This was followed by the addition of NaH (1.97 g, 49.255 mmol, 3.00 equiv, 60%), in portions at 0 degrees C. The resulting solution was stirred for 0.5 h at 0 degrees C. To this was added 5-bromo-6-fluoro-1-[[2-(trimethylsilyl)ethoxy]methyl]-1H-pyrrolo[2,3-b]pyridine (5.66 g, 16.392 mmol, 1 equiv). The resulting solution was stirred overnight at 50 degrees C. in an oil bath. The reaction mixture was cooled to 25 degree C. The reaction was then quenched by the addition of 500 mL of NH₄Cl. The resulting solution was extracted with 2×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 6 g (64.37%) of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide as colorless oil. ¹H NMR (300 MHz, CDCl₃-d,ppm) δ8.04 (s, 1H), 7.74-7.63 (m, 2H), 7.23-7.11 (m, 3H), 6.41 (d, J=3.6 Hz, 1H), 5.56 (d, J=3.3 Hz, 2H), 5.89-5.33 (m, 1H), 3.59-3.46 (m, 2H), 3.17 (t, J=6.0 Hz, 2H), 2.37 (s, 3H), 2.08-1.78 (m, 2H), 1.37 (d, J=6.2 Hz, 3H), 0.94-0.85 (m, 2H),-0.06 (s, 9H).

Synthesis of 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene. Into a 250-mL round-bottom flask purged and maintained with an inert atmosphere of nitrogen, was placed N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide (6 g, 10.552 mmol, 1 equiv), DMSO (60 mL), pyridine-2-carboxylic acid (1.04 g, 8.448 mmol, 0.80 equiv), CuI (2.41 g, 12.654 mmol, 1.20 equiv), K2CO₃ (4.38 g, 31.692 mmol, 3.00 equiv). The resulting solution was stirred for 2 days at 125 degrees C. in an oil bath. The reaction mixture was cooled to 25 degree C. The resulting solution was diluted with 500 mL of water. The resulting solution was extracted with 3×200 mL of ethyl acetate and the organic layers combined. The resulting mixture was washed with 2×1000 ml of brine. The mixture was dried over anhydrous sodium sulfate and concentrated under vacuum. The residue was applied onto a silica gel column with ethyl acetate/petroleum ether (1:3). This resulted in 2.8 g (54.41%) of 13-methyl-10-(4-methylbenzenesulfonyl)-4-[[2-(trimethylsilyl) ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, CDCl₃-d,ppm) S 8.10 (s, 1H), 7.53-7.40 (m, 2H), 7.30 (d, J=3.6 Hz, 1H), 7.24-7.12 (m, 2H), 6.50 (d, J=3.6 Hz, 1H), 5.65 (d, J=10.7 Hz, 1H), 5.49 (d, J=10.7 Hz, 1H), 4.37-4.23 (m, 1H), 3.94-3.80 (m, 1H), 3.62-3.36 (m, 3H), 2.37 (s, 3H), 1.89-1.64 (m, 2H), 1.23 (d, J=6.3 Hz, 3H), 1.01-0.76 (m, 2H),-0.07 (s, 9H).

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene; Into a 250-mL round-bottom flask purged and maintained with an inert atmosphere of nitrogen, was placed Na (793.5 mg, 34.51 mmol, 6.01 equiv), naphthalene (4.42 g, 34.48 mmol, 6.01 equiv), DME (20 mL).

The mixture was stirred at room temperature for 40 min until the formation of Na/naphthalene was complete. Another 250-mL round-bottom flask purged and maintained with an inert atmosphere of nitrogen, was placed 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene (2.8 g, 5.741 mmol, 1 equiv), THE (20 mL). This was followed by the addition of above solution at ˜78 degrees C. The resulting solution was stirred for 2 hr at room temperature. The reaction was then quenched by the addition of 500 mL of NH₄Cl.

The resulting solution was extracted with 3×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 1.2 g (62.67%) of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, DMSO-d₆,ppm) δ7.43-7.26 (m, 2H), 6.27 (d, J=3.5 Hz, 1H), 5.43 (d, J=3.4 Hz, 2H), 5.13 (s, 1H), 4.14-4.04 (m, 1H), 3.58-3.39 (m, 3H), 3.28-3.10 (m, 1H), 2.95-2.77 (m, 1H), 2.09-1.85 (m, 1H), 1.85-1.64 (m, 1H), 1.37 (d, J=6.3 Hz, 3H), 0.88-078 (m, 2H),-0.09 (s, 9H).

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene: The crude product (0.5 g) was purified by Chiral-Prep-HPLC with the following conditions: Instrument Name: SHIMADZU LC-20AD, LC parameters: Pump Mode: Binary gradient, Start Conc. of Pump B: 50.0%, Total Flow: 15 mL/min, Phase A: n-Hexane (0.1% DEA), Phase B: Eethanol, Column Name: CHIRALpak IA-3, Length: 50 mm, Internal Diameter: 4.6 mm, Particle Size: 3.0 um, Column Temp: 25° C., PDA Model: SPD-M20A, Wavelength: from 190 nm to 500 nm. This resulted in 220 mg (peak 1 Assumed R) [a]=−6.78° (C=0.129 g/100 mL in CH₂Cl2, T=27° C.) of (13R or S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil and 230 mg (peak 2 Assumed S) [a]=+11.84° (C=0.106 g/100 mL in CH₂Cl2, T=27° C.) of (13S or R)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil.

Synthesis of methyl 4-(4-[[2-(4-chlorophenyl)-4,4-dimethylcyclohex-1-en-1-yl]methyl] piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate: Into a 40-mL vial purged and maintained with an inert atmosphere of nitrogen, was placed (13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene (210.00 mg, 0.630 mmol, 1.00 equiv), toluene (5.00 mL), methyl 2-bromo-4-(4-[[2-(4-chlorophenyl)-4,4-dimethylcyclohex-1-en-1-yl]methyl]piperazin-1-yl)benzoate (669.87 mg, 1.259 mmol, 2.00 equiv), Cs₂CO₃ (1025.80 mg, 3.148 mmol, 5.00 equiv), Xantphos Pd 2G Precatalyst (111.70 mg, 0.126 mmol, 0.20 equiv). The resulting solution was stirred overnight at 110 degrees C. in an oil bath. The reaction mixture was cooled to room temperature. The resulting mixture was concentrated under vacuum. The residue was applied onto a silica gel column with ethyl acetate/petroleum ether (1:1). This resulted in 240 mg (31.09%) of methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0 \hat{ }[3,7]]tetradeca-1(9),2,5,7-tetraen-10-yl]benzoate as yellow oil. LC-MS: (ES, m/z): M+1=784.

Synthesis of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl]benzoic acid: Into a 8-mL round-bottom flask, was placed methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate (200 mg, 0.255 mmol, 1.00 equiv), H₂O (1.00 mL), MeOH (2.00 mL), dioxane (2.00 mL), NaOH (61.18 mg, 1.530 mmol, 6.00 equiv). The resulting solution was stirred for 12 h at 70 degrees C. The resulting mixture was concentrated. The pH value of the solution was adjusted to 5 with HCl (1 mol/L). The solids were collected by filtration. This resulted in 180 mg (91.64%) of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl]benzoic acid as a white solid. H-NMR-PH-PHNW-4-82-1: (CDCl₃, 300 ppm): δ8.17 (s, 1H), 7.28 (s, 1H), 7.00-6.79 (m, 4H), 6.35 (s, 1H), 5.70-5.67 (d, J=9 Hz, 1H), 5.56-5.52 (d, J=12 Hz, 1H), 4.38-4.35 (m, 1H), 3.81 (s, 1H), 3.58-3.56 (m, 4H), 3.26 (s, 3H), 2.84 (s, 1H), 2.35 (s, 3H), 2.24 (s, 2H), 2.12 (s, 2H), 2.04 (s, 2H), 1.60 (s, 6H), 1.28 (s, 1H), 1.01 (s, 7H), 0.98-0.95 (m, 3H), 0.00 (s, 9H).

Synthesis of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl]-N-(3-nitro-4-[[(oxan-4-yl)methyl]amino]benzenesulfonyl)benzamide: Into a 8-mL round-bottom flask, was placed 4-(4-[[2-(4-chlorophenyl)-4,4-dimethylcyclohex-1-en-1-yl]methyl] piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoic acid (100.00 mg, 0.130 mmol, 1.00 equiv), 3-nitro-4-[[(oxan-4-yl)methyl]amino]benzene-1-sulfonamide (40.93 mg, 0.130 mmol, 1 equiv), DCM (3 mL), DMAP (63.42 mg, 0.519 mmol, 4 equiv), EDCI (49.76 mg, 0.260 mmol, 2.00 equiv). The resulting solution was stirred for 12 hr at room temperature. The resulting mixture was concentrated. The residue was applied onto a silica gel column with ethyl acetate/petroleum ether (0:1-1:0). This resulted in 80 mg (57.72%) of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide as a yellow solid. LC-MS: (ES, m/z): M+1=1066.46.

Synthesis of 4-(4-[[2-(4-chlorophenyl)-4,4-dimethylcyclohex-1-en-1-yl]methyl]piperazin-1-yl)-2-[(13S)-13-methyl-14-oxa-2,4,10-triazatricyclo[7.5.0.0\hat{ }[3,7]]tetradeca-1(9),2,5,7-tetraen-10-yl]-N-(3-nitro-4-[[(oxan-4-yl)methyl]amino]benzenesulfonyl)benzamide: Into a 8-mL round-bottom flask, was placed 4-(4-[[2-(4-chlorophenyl)-4,4-dimethylcyclohex-1-en-1-yl] methyl]piperazin-1-yl)-2-[(13S)-13-methyl-4-[[2-(trimethylsilyl)ethoxy]methyl]-14-oxa-2,4,10-triazatricyclo [7.5.0.0\hat{ }[3,7]]tetradeca-1(9),2,5,7-tetraen-10-yl]-N-(3-nitro-4-[[(oxan-4-yl)methyl]amino]benzenesulfonyl) benzamide (80.00 mg, 0.075 mmol, 1.00 equiv), THE (5 mL), TBAF (391.77 mg, 1.498 mmol, 20.00 equiv), ethane-1,2-diamine (90.05 mg, 1.498 mmol, 20 equiv). The resulting solution was stirred for 48 hr at 70 degrees C. The resulting mixture was concentrated. The crude product was purified by Prep-HPLC with the following conditions (Waters-2767): Column, X-bridge RP18, Sum, 19*100 mm; mobile phase, 0.03% ammonia in water (0.03% NH₄HCO₃ & NH₄OH) and CH₃CN (32% CH₃CN up to 52% in 6 min); Detector, UV 254 nm. This resulted in 25 mg (35.59%) of 4-(4-[[2-(4-chlorophenyl)-4,4-dimethylcyclohex-1-en-1-yl]methyl]piperazin-1-yl)-2-[(13S)-13-methyl-14-oxa-2,4,10-triazatricyclo [7.5.0.0\hat{ }[3,7]]tetradeca-1(9),2,5, 7-tetraen-10-yl]-N-(3-nitro-4-[[(oxan-4-yl)methyl]amino] benzenesulfonyl) benzamide as a yellow solid. LC-MS-PH-PHNW-4-77-OB: (ES, m/z): M+1=937.54 The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient. H-NMR-PH-PHNW-4-77-OB: (300 MHz, Chloroform-d) δ 8.84-8.78 (m, 2H), 8.34 (s, 1H), 7.98-7.96 (d, J=6.0 Hz, 2H), 7.28-7.27 (m, 1H), 7.17-7.15 (m, 1H), 7.04-6.94 (m, 3H), 6.76-6.73 (m, 2H), 6.64 (s, 1H), 6.22 (s, 1H), 4.02-3.93 (m, 3H), 3.46-3.30 (m, 7H), 3.10 (s, 2H), 2.85 (s, 2H), 2.37 (s, 4H), 2.25 (s, 2H), 2.15-2.03 (m, 3H), 185-1.80 (m, 1H), 1.70 (s, 4H), 1.60 (s, 2H), 1.50-1.46 (m, 4H), 1.00 (s, 6H).

### Compound 3-37: Synthesis of 4-(4-[[2-(4-chlorophenyl)-4,4-dimethylcyclohex-1-en-1-yl]methyl]piperazin-1-yl)-N-[4-([[(2S)-1,4-dioxan-2-yl]methyl]amino)-3-nitrobenzenesulfonyl]-2-[(13S)-13-methyl-4-[[2-(trimethylsilyl ethoxy]methyl]-14-oxa-2,4,10-triazatricyclo[7.5.0.0\hat{ }[3,7]] tetradeca-1(9),2,5,7-tetraen-10-yl]benzamide, and Synthesis of 4-(4-[[2-(4-chlorophenyl)-4,4-dimethylcyclohex-1-en-1-yl]methyl]piperazin-1-yl)-N-(4-[[(1,4-dioxan-2-yl)methyl] amino]-3-nitrobenzenesulfonyl)-2-[(13S)-13-methyl-14-oxa-2,4,10-triazatricyclo[7.5.0.0\hat{ }[3,7]]tetradeca-1(9),2,5,7-tetraen-10-yl]benzamide

Synthesis of N-(3-hydroxybutyl)-4-methylbenzene-1-sulfonamide: Into a 250-mL round-bottom flask purged and maintained with an inert atmosphere of nitrogen, was placed 4-aminobutan-2-ol hydrochloride (4 g, 31.847 mmol, 1 equiv), DCM (40 mL), TEA (3.56 g, 35.181 mmol, 1.10 equiv). This was followed by the addition of 4-methylbenzene-1-sulfonyl chloride (6.08 g, 31.893 mmol, 1.00 equiv) in portions at 0 degrees C. The resulting solution was stirred for 2 h at 25 degrees C. The resulting mixture was concentrated under vacuum. The residue was applied onto a silica gel column and eluted with ethyl acetate/petroleum ether (1:1). This resulted in 4.4 g (56.78%) of N-(3-hydroxybutyl)-4-methylbenzene-1-sulfonamide as colorless oil. ¹H NMR (300 MHz, CDCl₃-d,ppm) δ7.82-7.73 (d, J=9.0 Hz, 2H), 7.39-7.30 (d, J=9.0 Hz, 2H), 4.06-3.82 (m, 1H), 3.24-3.16 (m, 1H), 3.06-3.00 (m, 1H), 2.45 (s, 3H), 1.70-1.55 (m, 2H), 1.20 (d, J=6.2 Hz, 3H). The measurements of the NMR spectra were done with Bruker AvanceIII HD 300 MHz with a probe head of BBOF.

Synthesis of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide: Into a 250-mL 3-necked round-bottom flask purged and maintained with an inert atmosphere of nitrogen, was placed N-(3-hydroxybutyl)-4-methylbenzene-1-sulfonamide (4.4 g, 18.083 mmol, 1.10 equiv), THE (60 mL). This was followed by the addition of NaH (1.97 g, 49.255 mmol, 3.00 equiv, 60%), in portions at 0 degrees C. The resulting solution was stirred for 0.5 h at 0 degrees C. To this was added 5-bromo-6-fluoro-1-[[2-(trimethylsilyl)ethoxy]methyl]-1H-pyrrolo[2,3-b]pyridine (5.66 g, 16.392 mmol, 1 equiv). The resulting solution was stirred overnight at 50 degrees C. in an oil bath. The reaction mixture was cooled to 25 degree C. The reaction was then quenched by the addition of 500 mL of aq. NH₄Cl. The resulting solution was extracted with 2×200 mL of ethyl acetate and the organic layers combined. The resulting mixture was washed with 1×500 ml of brine. The mixture was dried over anhydrous sodium sulfate and concentrated under vacuum. The residue was applied onto a silica gel column and eluted with ethyl acetate/petroleum ether (1:3). This resulted in 6 g (64.37%) of N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide as colorless oil. ¹H NMR (300 MHz, CDCl₃-d,ppm) δ8.04 (s, 1H), 7.74-7.63 (m, 2H), 7.23-7.11 (m, 3H), 6.41 (d, J=3.6 Hz, 1H), 5.56 (d, J=3.3 Hz, 2H), 5.89-5.33 (m, 1H), 3.59-3.46 (m, 2H), 3.17 (t, J=6.0 Hz, 2H), 2.37 (s, 3H), 2.08-1.78 (m, 2H), 1.37 (d, J=6.2 Hz, 3H), 0.94-0.85 (m, 2H),-0.06 (s, 9H). The measurements of the NMR spectra were done with Bruker AvanceIII HD 300 MHz with a probe head of BBOF.

### Synthesis of 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 250-mL round-bottom flask purged and maintained with an inert atmosphere of nitrogen, was placed N-[3-[(5-bromo-1-[[2-(trimethylsilyl)ethoxy]methyl]-1H-pyrrolo[2,3-b]pyridin-6-yl)oxy]butyl]-4-methylbenzene-1-sulfonamide (6 g, 10.552 mmol, 1 equiv), DMSO (60 mL), pyridine-2-carboxylic acid (1.04 g, 8.448 mmol, 0.80 equiv), CuI (2.41 g, 12.654 mmol, 1.20 equiv), K2CO₃ (4.38 g, 31.692 mmol, 3.00 equiv). The resulting solution was stirred for 2 days at 125 degrees C. in an oil bath. The reaction mixture was cooled to 25 degree C. The resulting solution was diluted with 500 mL of water. The resulting solution was extracted with 3×200 mL of ethyl acetate and the organic layers combined. The resulting mixture was washed with 2×1000 ml of brine. The mixture was dried over anhydrous sodium sulfate and concentrated under vacuum. The residue was applied onto a silica gel column and eluted with ethyl acetate/petroleum ether (1:3). This resulted in 2.8 g (54.41%) of 13-methyl-10-(4-methylbenzenesulfonyl)-4-[[2-(trimethylsilyl)ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, CDCl₃-d,ppm) δ8.10 (s, 1H), 7.53-7.40 (m, 2H), 7.30 (d, J=3.6 Hz, 1H), 7.24-7.12 (m, 2H), 6.50 (d, J=3.6 Hz, 1H), 5.65 (d, J=10.7 Hz, 1H), 5.49 (d, J=10.7 Hz, 1H), 4.37-4.23 (m, 1H), 3.94-3.80 (m, 1H), 3.62-3.36 (m, 3H), 2.37 (s, 3H), 1.89-1.64 (m, 2H), 1.23 (d, J=6.3 Hz, 3H), 1.01-0.76 (m, 2H),-0.07 (s, 9H). The measurements of the NMR spectra were done with Bruker AvanceIII HD 300 MHz with a probe head of BBOF

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 250-mL round-bottom flask purged and maintained with an inert atmosphere of nitrogen, was placed Na (793.5 mg, 34.51 mmol, 6.01 equiv), naphthalene (4.42 g, 34.48 mmol, 6.01 equiv), DME (20 mL). The mixture was stirred at room temperature for 40 min until the formation of Na/naphthalene was complete. Another 250-mL round-bottom flask purged and maintained with an inert atmosphere of nitrogen, was placed 13-methyl-10-(4-methylbenzenesulfonyl)-4-[[2-(trimethylsilyl)ethoxy]methyl]-14-oxa-2,4,10-triazatricyclo[7.5.0.0 \hat{ }[3,7]]tetradeca-1(9),2,5,7-tetraene (2.8 g, 5.741 mmol, 1 equiv), THE (20 mL). This was followed by the addition of above Na/naphthalene solution at ˜78 degrees C. The resulting solution was stirred for 3 hr at room temperature. The reaction was then quenched by the addition of 500 mL of NH₄Cl. The resulting solution was extracted with 3×200 mL of ethyl acetate and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:3). This resulted in 1.2 g (62.67%) of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as yellow oil. ¹H NMR (300 MHz, DMSO-d₆,ppm) δ7.43-7.26 (m, 2H), 6.27 (d, J=3.5 Hz, 1H), 5.43 (d, J=3.4 Hz, 2H), 5.13 (s, 1H), 4.14-4.04 (m, 1H), 3.58-3.39 (m, 3H), 3.28-3.10 (m, 1H), 2.95-2.77 (m, 1H), 2.09-1.85 (m, 1H), 1.85-1.64 (m, 1H), 1.37 (d, J=6.3 Hz, 3H), 0.88-078 (m, 2H),-0.09 (s, 9H). The measurements of the NMR spectra were done with Bruker AvanceIII HD 300 MHz with a probe head of BBOF.

Synthesis of 13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene The crude product (0.5 g) was purified by Chiral-Prep-HPLC with the following conditions: Instrument Name: SHIMADZU LC-20AD, LC parameters: Pump Mode: Binary gradient, Start Conc. of Pump B: 50.0%, Total Flow: 15 mL/min, Phase A: n-Hexane (0.1% DEA), Phase B: Eethanol, Column Name: CHIRALpak IA-3, Length: 50 mm, Internal Diameter: 4.6 mm, Particle Size: 3.0 um, Column Temp: 25° C., PDA Model: SPD-M20A, Wavelength: from 190 nm to 500 nm. This resulted in 220 mg (peak 1, assumed as R) [a]=−6.78° (C=0.129 g/100 mL in CH₂Cl2, T=27° C.) of (13R or S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil and 230 mg (peak 2, assumed as S) [a]=+11.84° (C=0.106 g/100 mL in CH₂Cl2, T=27° C.) of (13S or R)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene as a yellow oil.

Synthesis of methyl 4-(4-[[2-(4-chlorophenyl)-4,4-dimethylcyclohex-1-en-1-yl]methyl]piperazin-1-yl)-2-[(13R)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate Into a 40-mL vial purged and maintained with an inert atmosphere of nitrogen, was placed (13R)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene (210.00 mg, 0.630 mmol, 1.00 equiv), toluene (5.00 mL), methyl 2-bromo-4-(4-[[2-(4-chlorophenyl)-4,4-dimethylcyclohex-1-en-1-yl]methyl]piperazin-1-yl)benzoate (669.87 mg, 1.259 mmol, 2.00 equiv), Cs₂CO₃ (1025.80 mg, 3.148 mmol, 5.00 equiv), Xantphos Pd 2G Precatalyst (111.70 mg, 0.126 mmol, 0.20 equiv). The resulting solution was stirred overnight at 110 degrees C. in an oil bath. The reaction mixture was cooled to room temperature. The resulting mixture was concentrated under vacuum. The residue was applied onto a silica gel column and eluted with ethyl acetate/petroleum ether (1:1). This resulted in 240 mg (31.09%) of methyl 4-(4-[[2-(4-chlorophenyl)-4,4-dimethylcyclohex-1-en-1-yl]methyl]piperazin-1-yl)-2-[(13R)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate as yellow oil. LC-MS: (ES, m/z): M+1=784.

### Synthesis of methyl 4-(4-[[2-(4-chlorophenyl)-4,4-dimethylcyclohex-1-en-1-yl]methyl]piperazin-1-yl)-2-[(13R)-13-methyl-14-oxa-2,4,10-triazatricyclo[7.5.0.0\hat{ }[3,7]]tetradeca-1(9),2,5,7-tetraen-10-yl]benzoate: Into a 40-mL vial, was placed methyl 4-(4-[[2-(4-chlorophenyl)-4,4-dimethylcyclohex-1-en-1-yl]methyl]piperazin-1-yl)-2-[(13R)-13-methyl-4-[[2-(trimethylsilyl)ethoxy]methyl]-14-oxa-2,4,10-triazatricyclo[7.5.0.0 \hat{ }[3,7]]tetradeca-1(9),2,5,7-tetraen-10-yl]benzoate (240.00 mg, 64%), THE (10.00 mL), TBAF.3H₂O (3.00 g), ethylenediamine (1.50 g). The resulting solution was stirred for 3 days at 70 degrees C. in an oil bath. The reaction mixture was cooled to room temperature. The resulting mixture was concentrated under vacuum. The residue was applied onto a silica gel column with ethyl acetate/petroleum ether (2:1). This resulted in 80 mg (37.47%) of methyl 4-(4-[[2-(4-chlorophenyl)-4,4-dimethylcyclohex-1-en-1-yl]methyl]piperazin-1-yl)-2-[(13R)-13-methyl-14-oxa-2,4,10-triazatricyclo[7.5.0.0\hat{ }[3,7]]tetradeca-1(9),2,5,7-tetraen-10-yl]benzoate as a white solid. LC-MS: (ES, m/z): M+1=654

Synthesis of 4-(4-[[2-(4-chlorophenyl)-4,4-dimethylcyclohex-1-en-1-yl]methyl]piperazin-1-yl)-2-[(13R)-13-methyl-14-oxa-2,4,10-triazatricyclo[7.5.0.0\hat{ }[3,7]]tetradeca-1(9),2,5,7-tetraen-10-yl]benzoic acid: Into a 40-mL vial, was placed methyl 4-(4-[[2-(4-chlorophenyl)-4,4-dimethylcyclohex-1-en-1-yl]methyl]piperazin-1-yl)-2-[(13R)-13-methyl-14-oxa-2,4,10-triazatricyclo[7.5.0.0\hat{ }[3,7]]tetradeca-1(9),2,5,7-tetraen-10-yl]benzoate (80.00 mg, 0.073 mmol, 1.00 equiv, 60%), MeOH (3.00 mL), dioxane (3.00 mL), 4M NaOH (0.60 mL, 2.400 mmol, 32.71 equiv). The resulting solution was stirred overnight at 70 degrees C. in an oil bath. The reaction mixture was cooled to room temperature. The resulting mixture was concentrated under vacuum. The pH value of the solution was adjusted to 6-7 with HCl (2 mol/L). The resulting solution was extracted with 2×30 mL of ethyl acetate and the organic layers combined. The resulting mixture was washed with 1×200 mL of brine. The mixture was dried over anhydrous sodium sulfate and concentrated under vacuum. The residue was purified by Prep-TLC with ethyl acetate. This resulted in 45 mg (47.90%) of 4-(4-[[2-(4-chlorophenyl)-4,4-dimethylcyclohex-1-en-1-yl]methyl] piperazin-1-yl)-2-[(13R)-13-methyl-14-oxa-2,4,10-triazatricyclo[7.5.0.0\hat{ }[3,7]]tetradeca-1(9),2,5,7-tetraen-10-yl]benzoic acid as a white solid.LC-MS: (ES, m/z): M+1=640.

Synthesis of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(13R)-13-methyl-14-oxa-2,4,10-triazatricyclo[7.5.0.0\hat{ }[3,7]]tetradeca-1(9),2,5,7-tetraen-10-yl]benzamide Into a 40-mL vial, was placed 4-(4-[[2-(4-chlorophenyl)-4,4-dimethylcyclohex-1-en-1-yl]methyl]piperazin-1-yl)-2-[(13R)-13-methyl-14-oxa-2,4,10-triazatricyclo[7.5.0.0 \hat{ }[3,7]]tetradeca-1(9),2,5,7-tetraen-10-yl]benzoic acid (40.00 mg, 0.062 mmol, 1.00 equiv), DCM (10.00 mL), 4-[[(2S)-1,4-dioxan-2-ylmethyl]amino]-3-nitrobenzenesulfonamide (20.00 mg, 0.063 mmol, 1.01 equiv), EDCI (24.00 mg, 0.125 mmol, 2.00 equiv), DMAP (31.00 mg, 0.254 mmol, 4.06 equiv). The resulting solution was stirred overnight at 25 degrees C. The resulting mixture was concentrated under vacuum. The crude product was purified by Prep-HPLC with the following conditions (2 #SHIMADZU (HPLC-01)): Column, XBridge Prep C18 OBD Column, 5 um,19*150 mm; mobile phase, ACN and Water (0.05% NH₃.H₂O) (20% Phase B up to 75% in 1 min, up to 95% in 7 min, hold 95% in 1 min, down to 20% in 1 min); Detector, 254/220 nm. This resulted in 12.6 mg (21.47%) of 4-(4-[[2-(4-chlorophenyl)-4,4-dimethylcyclohex-1-en-1-yl]methyl]piperazin-1-yl)-N-(4-[[(2S)-1,4-dioxan-2-ylmethyl]amino]-3-nitrobenzenesulfonyl)-2-[(13R)-13-methyl-14-oxa-2,4,10-triazatricyclo[7.5.0.0\hat{ }[3,7]]tetradeca-1(9),2,5,7-tetraen-10-yl]benzamide as a yellow solid.LC-MS: (ES, m/z): M+1=939, R.T=3.510 min. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 40° C.; flow: 1.5 mL/min. ¹H NMR (300 MHz, DMSO-d₆,ppm) δ11.17 (s, 1H), 8.47 (d, J=2.2 Hz, 2H), 7.63 (dd, J=9.2, 2.3 Hz, 1H), 7.49 (d, J=8.6 Hz, 1H), 7.42-7.31 (m, 2H), 7.21 (t, J=2.9 Hz, 1H), 7.13-7.02 (m, 2H), 6.96-6.79 (m, 2H), 6.70 (s, 2H), 6.12 (dd, J=3.4, 1.8 Hz, 1H), 4.25 (s, 1H), 3.92-3.42 (m, 11H), 3.30-3.10 (m, 4H), 2.78 (s, 2H), 2.22 (d, J=18.1 Hz, 6H), 1.95 (d, J=20.3 Hz, 4H), 1.41 (d, J=6.1 Hz, 5H), 0.95 (s, 6H). The measurements of the NMR spectra were done with Bruker AvanceIII HD 300 MHz with a probe head of BBOF.

### Synthesis of methyl 4-(4-[[2-(4-chlorophenyl)-4,4-dimethylcyclohex-1-en-1-yl]methyl] piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate Into a 40-mL vial purged and maintained with an inert atmosphere of nitrogen, was placed (13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraene (210.00 mg, 0.630 mmol, 1.00 equiv), toluene (5.00 mL), methyl 2-bromo-4-(4-[[2-(4-chlorophenyl)-4,4-dimethylcyclohex-1-en-1-yl]methyl]piperazin-1-yl)benzoate (669.87 mg, 1.259 mmol, 2.00 equiv), Cs₂CO₃ (1025.80 mg, 3.148 mmol, 5.00 equiv), Xantphos Pd 2G Precatalyst(111.70 mg, 0.126 mmol, 0.20 equiv). The resulting solution was stirred overnight at 110 degrees C. in an oil bath. The reaction mixture was cooled to room temperature. The resulting mixture was concentrated under vacuum. The residue was applied onto a silica gel column with ethyl acetate/petroleum ether (1:1). This resulted in 240 mg (31.09%) of methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0 \hat{ }[3,7]]tetradeca-1(9),2,5,7-tetraen-10-yl]benzoate as yellow oil.LC-MS: (ES, m/z): M+1=784

Synthesis of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl]benzoic acid: Into a 8-mL round-bottom flask, was placed methyl 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]benzoate (200 mg, 0.255 mmol, 1.00 equiv), H₂O (1.00 mL), MeOH (2.00 mL), dioxane (2.00 mL), NaOH (61.18 mg, 1.530 mmol, 6.00 equiv). The resulting solution was stirred for 12 h at 70 degrees C. The resulting mixture was concentrated. The pH value of the solution was adjusted to 5 with HCl (1 mol/L). The solids were collected by filtration. This resulted in 180 mg (91.64%) of 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl]benzoic acid as a white solid. H-NMR-PH-PHNW-4-82-1: (CDCl₃, 300 ppm): δ8.17 (s, 1H), 7.28 (s, 1H), 7.00-6.79 (m, 4H), 6.35 (s, 1H), 5.70-5.67 (d, J=9 Hz, 1H), 5.56-5.52 (d, J=12 Hz, 1H), 4.38-4.35 (m, 1H), 3.81 (s, 1H), 3.58-3.56 (m, 4H), 3.26 (s, 3H), 2.84 (s, 1H), 2.35 (s, 3H), 2.24 (s, 2H), 2.12 (s, 2H), 2.04 (s, 2H), 1.60 (s, 6H), 1.28 (s, 1H), 1.01 (s, 7H), 0.98-0.95 (m, 3H), 0.00 (s, 9H). The measurements of the NMR spectra were done with Bruker AvanceIII HD 300 MHz with a probe head of BBOF.

Synthesis of 4-(4-[[2-(4-chlorophenyl)-4,4-dimethylcyclohex-1-en-1-yl]methyl]piperazin-1-yl)-N-[4-([[(2S)-1,4-dioxan-2-yl]methyl]amino)-3-nitrobenzenesulfonyl]-2-[(13S)-13-methyl-4-[[2-(trimethylsilyl ethoxy]methyl]-14-oxa-2,4,10-triazatricyclo[7.5.0.0\hat{ }[3,7]] tetradeca-1(9),2,5,7-tetraen-10-yl]benzamide: Into a 40-mL round-bottom flask, was placed 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0 \hat{ }[3,7]]tetradeca-1(9),2,5,7-tetraen-10-yl]benzoic acid (180.00 mg, 0.234 mmol, 1.00 equiv), 4-[[(1,4-dioxan-2-yl)methyl]amino]-3-nitrobenzene-1-sulfonamide (88.96 mg, 0.280 mmol, 1.20 equiv), DCM (20.00 mL), DMAP (114.16 mg, 0.934 mmol, 4 equiv), EDC.HCl (89.57 mg, 0.467 mmol, 2 equiv). The resulting solution was stirred for 12 h at room temperature. The resulting mixture was concentrated. The residue was applied onto a silica gel column with ethyl acetate/petroleum ether (0:1-1:1). This resulted in 180 mg (72.02%) of 4-(4-[[2-(4-chlorophenyl)-4,4-dimethylcyclohex-1-en-1-yl]methyl]piperazin-1-yl)-N-[4-([[(2S)-1,4-dioxan-2-yl]methyl]amino)-3-nitrobenzenesulfonyl]-2-[(13S)-13-methyl-4-[[2-(trimethylsilyl ethoxy]methyl]-14-oxa-2,4,10-triazatricyclo[7.5.0.0\hat{ }[3,7]] tetradeca-1(9),2,5,7-tetraen-10-yl]benzamide as a yellow solid. LC-MS-PH-PHNW-4-82-2: (ES, m/z): M+1=1069.6. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient.

Synthesis of 4-(4-[[2-(4-chlorophenyl)-4,4-dimethylcyclohex-1-en-1-yl]methyl]piperazin-1-yl)-N-(4-[[(1,4-dioxan-2-yl)methyl] amino]-3-nitrobenzenesulfonyl)-2-[(13S)-13-methyl-14-oxa-2,4,10-triazatricyclo[7.5.0.0\hat{ }[3,7]]tetradeca-1(9),2,5,7-tetraen-10-yl]benzamide: Into a 40-mL round-bottom flask, was placed 4-(4-[[2-(4-chlorophenyl)-4,4-dimethylcyclohex-1-en-1-yl]methyl]piperazin-1-yl)-2-[(13S)-13-methyl-4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl]-N-(3-nitro-4-[[(oxan-3-yl)methyl]amino]benzenesulfonyl)benzamide (180.00 mg, 0.169 mmol, 1.00 equiv), THE (10.00 mL), ethane-1,2-diamine (202.61 mg, 3.371 mmol, 20.00 equiv), TBAF (881.48 mg, 3.371 mmol, 20 equiv). The resulting solution was stirred for 12 h at 70 degrees C. The resulting mixture was concentrated. The crude product was purified by Prep-HPLC with the following conditions (Waters-2767): Column, X-bridge RP18, Sum, 19*100 mm; mobile phase, 0.03% ammonia in water (0.03% NH₄HCO₃ & NH₄OH) and CH₃CN (32% CH₃CN up to 52% in 6 min); Detector, UV 254 nm. This resulted in 30 mg (18.94%) of 4-(4-[[2-(4-chlorophenyl)-4,4-dimethylcyclohex-1-en-1-yl]methyl]piperazin-1-yl)-N-(4-[[(1,4-dioxan-2-yl)methyl] amino]-3-nitrobenzenesulfonyl)-2-[(13S)-13-methyl-14-oxa-2,4,10-triazatricyclo[7.5.0.0\hat{ }[3,7]]tetradeca-1(9),2,5,7-tetraen-10-yl]benzamide as a yellow solid.LC-MS-PH-PHNW-4-82-OB: (ES, m/z): M+1=939.52. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient H-NMR-PH-PHNW-4-82-OB: (CDCl₃, 300 ppm): δ8.84-8.83 (m, 2H), 8.45 (s, 1H), 7.99-7.94 (m, 2H), 7.15 (s, 1H), 6.97-6.93 (m, 2H), 6.75-6.62 (m, 3H), 6.20 (s, 1H), 3.94-3.62 (m, 7H), 3.49-3.42 (m, 2H), 3.30 (s, 5H), 2.85 (s, 2H), 2.37 (s, 3H), 2.26 (s, 2H), 2.11-2.03 (m, 3H), 1.68-1.48 (m, 4H), 1.28 (s, 1H), 1.00 (s, 6H).

### Compound 3-38: Preparation of 4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)-2-[15-oxa-2,4,10-triazatricyclo[7.6.0.0\hat{ }[3,7]]pentadeca-1(9),2,5,7-tetraen-10-yl]benzamide

Synthesis of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-15-oxa-2,4,10-triazatricyclo[7.6.0.0\hat{ }[3,7]]pentadeca-1(9),2,5,7-tetraen-10-yl)benzoate: Into a 40-mL vial purged and maintained with an inert atmosphere of nitrogen, was placed 4-[[2-(trimethylsilyl)ethoxy]methyl]-15-oxa-2,4,10-triazatricyclo[7.6.0.0\hat{ }[3,7]]pentadeca-1(9),2,5,7-tetraene (125 mg, 0.375 mmol, 1 equiv), toluene (5 mL), methyl 2-bromo-4-(4-[[2-(4-chlorophenyl)-4,4-dimethylcyclohex-1-en-1-yl]methyl]piperazin-1-yl)benzoate (359 mg, 0.675 mmol, 1.80 equiv), Cs₂CO₃ (610 mg, 1.874 mmol, 5 equiv), XantPhos Pd 2G (266 mg, 0.300 mmol, 0.8 equiv). The resulting solution was stirred overnight at 110° C. in an oil bath. The reaction mixture was cooled to room temperature. The resulting mixture was concentrated under vacuum. The residue was applied onto a silica gel column with ethyl acetate/petroleum ether (1:1). This resulted in 140 mg (47.61%) of methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-15-oxa-2,4,10-triazatricyclo[7.6.0.0\hat{ }[3,7]]pentadeca-1(9),2,5,7-tetraen-10-yl)benzoate as a yellow solid. LC-MS: (ES, m/z): M+H=784, R.T=1.236 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile; linear gradient from 5% acetonitrile to 100% acetonitrile in 2.0 minutes; Oven temperature 40° C.; flow: 1.5 mL/min.

Synthesis of methyl 4-(4-[[2-(4-chlorophenyl)-4,4-dimethylcyclohex-1-en-1-yl]methyl]piperazin-1-yl)-2-[15-oxa-2,4,10-triazatricyclo[7.6.0.0\hat{ }[3,7]]pentadeca-1(9),2,5,7-tetraen-10-yl]benzoate. Into a 40-mL vial, was placed methyl 4-(4-[[2-(4-chlorophenyl)-4,4-dimethylcyclohex-1-en-1-yl]methyl]piperazin-1-yl)-2-(4-[[2-(trimethylsilyl)ethoxy]methyl]-15-oxa-2,4,10-triazatricyclo[7.6.0.0\hat{ }[3,7]]pentadeca-1(9),2,5,7-tetraen-10-yl)benzoate (140 mg, 0.178 mmol, 1 equiv), THE (10 mL), ethane-1,2-diamine (500 mg, 8.319 mmol, 46.62 equiv), TBAF.3H₂O (1 g). The resulting solution was stirred overnight at 70° C. in an oil bath. The reaction mixture was cooled to room temperature. The resulting solution was diluted with 100 mL of water. The resulting solution was extracted with 2×50 mL of ethyl acetate and the organic layers combined. The resulting mixture was washed with 1×200 mL of brine. The mixture was dried over anhydrous sodium sulfate and concentrated under vacuum. The residue was purified by Prep-TLC with ethyl acetate/petroleum ether (2:1). This resulted in 60 mg (51.39%) of methyl 4-(4-[[2-(4-chlorophenyl)-4,4-dimethylcyclohex-1-en-1-yl]methyl]piperazin-1-yl)-2-[15-oxa-2,4,10-triazatricyclo[7.6.0.0\hat{ }[3,7]]pentadeca-1(9),2,5,7-tetraen-10-yl]benzoate as a yellow solid. LC-MS: (ES, m/z): M+H=654, R.T=1.099 min. The measurements of the retention were done with a reversed phase column (C18). Shimadzu LCMS 2020; 50*3.0 Kinetex 2.6u XB-C18, 2.6 microm; Eluent A: water (0.05% TFA); Eluent B: Acetonitrile (0.05% TFA); linear gradient from 5% acetonitrile to 100% acetonitrile in 2.0 minutes; Oven temperature 40° C.; flow: 1.5 mL/min.

Synthesis of 4-(4-[[2-(4-chlorophenyl)-4,4-dimethylcyclohex-1-en-1-yl]methyl]piperazin-1-yl)-2-[15-oxa-2,4,10-triazatricyclo[7.6.0.0\hat{ }[3,7]]pentadeca-1(9),2,5,7-tetraen-10-yl]benzoic acid: Into a 40-mL vial, was placed methyl 4-(4-[[2-(4-chlorophenyl)-4,4-dimethylcyclohex-1-en-1-yl]methyl]piperazin-1-yl)-2-[15-oxa-2,4,10-triazatricyclo[7.6.0.0\hat{ }[3,7]]pentadeca-1(9),2,5,7-tetraen-10-yl]benzoate (60 mg, 0.092 mmol, 1 equiv), MeOH (3 mL), THE (1 mL), H₂O (1 mL), NaOH (37 mg, 0.917 mmol, 10.00 equiv). The resulting solution was stirred overnight at 60° C. in an oil bath. The reaction mixture was cooled to room temperature. The resulting mixture was concentrated under vacuum. The pH value of the solution was adjusted to 4-5 with HCl (2 mol/L). The resulting solution was extracted with 2×30 mL of ethyl acetate and the organic layers combined. The resulting mixture was washed with 1×100 mL of brine. The mixture was dried over anhydrous sodium sulfate and concentrated under vacuum. The residue was purified by Prep-TLC with ethyl acetate. This resulted in 16 mg (27.25%) of 4-(4-[[2-(4-chlorophenyl)-4,4-dimethylcyclohex-1-en-1-yl]methyl]piperazin-1-yl)-2-[15-oxa-2,4,10-triazatricyclo[7.6.0.0\hat{ }[3,7]]pentadeca-1(9),2,5,7-tetraen-10-yl]benzoic acid as a white solid. LC-MS: (ES, m/z): M+H=640, R.T=1.396 min. The measurements of the retention were done with a reversed phase column (C18). Shimadzu LCMS 2020; 50*3.0, Shinadzu shim-pack XR-ODS, 2.2 microm; Eluent A: water (0.05% TFA); Eluent B: Acetonitrile; linear gradient from 5% acetonitrile to 100% acetonitrile in 2.6 minutes; Oven temperature 40° C.; flow: 1.0 mL/min.

Synthesis of 4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)-2-[15-oxa-2,4,10-triazatricyclo[7.6.0.0\hat{ }[3,7]] pentadeca-1(9),2,5,7-tetraen-10-yl]benzamide. Into a 8-mL vial, was placed 4-(4-[[2-(4-chlorophenyl)-4,4-dimethylcyclohex-1-en-1-yl]methyl]piperazin-1-yl)-2-[15-oxa-2,4,10-triazatricyclo[7.6.0.0\hat{ }[3,7]]pentadeca-1(9),2,5,7-tetraen-10-yl]benzoic acid (16 mg, 0.025 mmol, 1 equiv), DCM (3 mL), 3-nitro-4-[[(oxan-4-yl)methyl]amino]benzene-1-sulfonamide (7.9 mg, 0.025 mmol, 1 equiv), EDCI (9.6 mg, 0.050 mmol, 2 equiv), DMAP (12.2 mg, 0.100 mmol, 4 equiv). The resulting solution was stirred overnight at room temperature. The resulting mixture was concentrated under vacuum. The crude product was purified by Prep-HPLC with the following conditions (2 #SHIMADZU (HPLC-01)): Column, X Bridge Prep C18 OBD Column, 5 um, 19*150 mm; mobile phase, ACN and Water (0.05% NH₃.H₂O) (20% Phase B up to 75% in 1 min, up to 95% in 7 min, hold 95% in 1 min, down to 20% in 1 min); Detector, 254 and 220 nm. This resulted in 4.5 mg (19.21%) of 4-(4-[[2-(4-chlorophenyl)-4,4-dimethylcyclohex-1-en-1-yl]methyl]piperazin-1-yl)-N-(3-nitro-4-[[(oxan-4-yl)methyl]amino]benzenesulfonyl)-2-[15-oxa-2,4,10-triazatricyclo[7.6.0.0\hat{ }[3,7]]pentadeca-1(9),2,5,7-tetraen-10-yl]benzamide as a yellow solid. LC-MS: (ES, m/z): M+1=937, R.T=2.522 min. The measurements of the retention were done with a reversed phase column (C18). Shimadzu LCMS 2020; SUPELCO Ascentis Sxpress C18, 50*3.0 mm, 2.7 microm; Eluent A: water (0.05% TFA); Eluent B: Acetonitrile; linear gradient from 5% acetonitrile to 100% acetonitrile in 5 minutes; Oven temperature 40° C.; flow: 1.5 mL/min.

### Compound 3-39: Preparation of 4-(4-[[4-(4-chlorophenyl)-6,6-dimethyl-2,5-dihydropyran-3-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide

Synthesis of 4-bromo-6,6-dimethyl-2,5-dihydropyran-3-carbaldehyde: Into a 250-mL 3-necked round-bottom flask, was added CHCl₃ (80.00 mL), dimethylformamide (7.13 g, 97.5 mmol, 2.5 equiv). This was followed by the addition of phosphorus tribromide (24.28 g, 89.7 mmol, 2.30 equiv) dropwise with stirring at 0° C. The resulting solution was stirred for 1 h at Rt. To this was added 2,2-dimethyloxan-4-one (5.00 g, 39.01 mmol, 1.00 equiv) at RT.. The resulting solution was allowed to react an additional 12 h at RT. The reaction was then quenched by the addition of 200 mL of water/ice. The pH value of the solution was adjusted to 5 with Na₂CO₃(s). The resulting solution was extracted with 3×50 mL of dichloromethane and the organic layers combined and concentrated. The residue was dissolved in 100 mL of EA. The resulting mixture was washed with 2×50 mL of brine. The mixture was dried over anhydrous sodium sulfate and concentrated. This resulted in 5.6 g (65.53%) of 4-bromo-6,6-dimethyl-2,5-dihydropyran-3-carbaldehyde as light yellow oil. ¹H NMR (300 MHz, DMSO-d₆, ppm) δ9.84 (s, 1H), 4.23 (t, J=2.5 Hz, 2H), 2.73 (t, J=2.5 Hz, 2H), 1.20 (s, 6H).

Synthesis of 4-(4-chlorophenyl)-6,6-dimethyl-2,5-dihydropyran-3-carbaldehyde: Into a 100-mL round-bottom flask purged and maintained with an inert atmosphere of nitrogen, was added 4-bromo-6,6-dimethyl-2,5-dihydropyran-3-carbaldehyde (5.40 g, 24.6 mmol, 1.00 equiv), DCE (50.00 mL), p-chloro-benzeneboronic acid (4.63 g, 29.6 mmol, 1.2 equiv), sodium methaneperoxoate sodium (5.27 g, 49.2 mmol, 2 equiv), Pd(PPh₃)₂Cl₂ (1.73 g, 2.46 mmol, 0.1 equiv), water (5 mL). The resulting solution was stirred for 12 h at 60° C. The reaction mixture was cooled and diluted with 50 ml water. The resulting solution was extracted with 3×50 mL of ethyl acetate and the organic layers combined. The resulting mixture was washed with 50 mL of brine. The mixture was dried over anhydrous sodium sulfate and concentrated. The residue was applied onto a silica gel column with ethyl acetate/petroleum ether (1:10-2:5). This resulted in 5.2 g (84.14%) of 4-(4-chlorophenyl)-6,6-dimethyl-2,5-dihydropyran-3-carbaldehyde as light yellow oil. LC-MS: (ES, m/z): M+1: 251.

Synthesis of methyl 2-bromo-4-(4-[[4-(4-chlorophenyl)-6,6-dimethyl-2,5-dihydropyran-3-yl]methyl]piperazin-1-yl)benzoate: Into a 100-mL 3-necked round-bottom flask, was added 4-(4-chlorophenyl)-6,6-dimethyl-2,5-dihydropyran-3-carbaldehyde (2.00 g, 7.9 mmol, 1.00 equiv), DCE (20.00 mL), methyl 2-bromo-4-(piperazin-1-yl)benzoate (2.36 g, 7.9 mmol, 1 equiv), Ti(Oi-Pr)₄ (6.74 g, 23.7 mmol, 3equiv). The resulting solution was stirred for 3 h at RT. This was followed by the addition of NaBH(OAc)₃ (3.35 g, 15.8 mmol, 2 equiv) in several batches at RT. The resulting solution was allowed to react for overnight at RT. The reaction was then quenched by the addition of 10 mL of. The resulting mixture was concentrated. The residue was applied onto a silica gel column with ethyl acetate/petroleum ether (1:10-2:5). This resulted in 3 g (70.44%) of methyl 2-bromo-4-(4-[[4-(4-chlorophenyl)-6,6-dimethyl-2,5-dihydropyran-3-yl]methyl]piperazin-1-yl)benzoate as light yellow oil. LC-MS: (ES, m/z): M+1: 533.

Synthesis of 2-[3-[(5-bromo-1-[[2-(trimethylsilyl)ethoxy]methyl]pyrrolo[2,3-b]pyridine-6-yl)oxy] propyl]isoindole-1,3-dione: Into a 1000-mL 3-necked round-bottom flask purged and maintained with an inert atmosphere of nitrogen, was placed 5-bromo-6-fluoro-1-[[2-(trimethylsilyl)ethoxy]methyl]pyrrolo[2,3-b]pyridine (53.50 g, 154.944 mmol, 1 equiv). This was followed by the addition of 2-(3-hydroxypropyl)isoindole-1,3-dione (31.80 g, 154.944 mmol, 1 equiv), in portions at RT. To this was added Dioxane (500.00 mL) at RT, NaH (9.30 g, 232.415 mmol, 1.50 equiv, 60%). The resulting solution was stirred for 4 h at 80° C. The reaction mixture was cooled with a water/ice bath. The reaction was then quenched by the addition of 500 mL AcOH/ice/water. The resulting solution was extracted with 2×500 mL of ethyl acetate. The resulting mixture was washed with 3×500 ml of Brine. The mixture was dried over anhydrous sodium sulfate and concentrated. The residue was applied onto a silica gel column with ethyl acetate/petroleum ether (0:1-1:10). This resulted in 58 g (70.56%) of 2-[3-[(5-bromo-1-[[2-(trimethylsilyl)ethoxy]methyl]pyrrolo[2,3-b]pyridin-6-yl)oxy]propyl]isoindole-1,3-dione as colorless oil. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min. LC-MS: (ES, m/z): M+1:554.

Synthesis of 3-[(5-bromo-1-[[2-(trimethylsilyl)ethoxy]methyl]pyrrolo[2,3-b]pyridin-6-yl)oxy] propan-1-amine: Into a 500-mL round-bottom flask, was placed ethoxy]methyl]propyl]isoindole-pyrrolo[2,3-b]pyridin-6-yl)oxy]2-[3-[(5-bromo-1-[[2-(trimethylsilyl)1,3-dione (58.00 g, 109.332 mmol, 1.00 equiv), EtOH (300.00 mL), NH₂NH₂.H₂O (68.42 g, 1093.32 mmol, 10 equiv, 80%). The resulting solution was stirred for 4 h at room temperature. The resulting mixture was concentrated. The reaction was then quenched by the addition of 200 mL of water. The resulting solution was extracted with 2×500 mL of ethyl acetate. The resulting mixture was washed with 2×300 ml of Brine. The mixture was dried over anhydrous sodium sulfate and concentrated. The residue was applied onto a silica gel column with ethyl acetate/petroleum ether (0:1-1:2). This resulted in 37.5 g (85.66%) of 3-[(5-bromo-1-[[2-(trimethylsilyl)ethoxy]methyl]pyrrolo[2,3-b]pyridin-6-yl)oxy]propan-1-amine as yellow oil.

Synthesis of 4-[[2-(trimethylsilyl)ethoxy]methyl]-14-oxa-2,4,10-triazatricyclo[7.5.0.0\hat{ }[3,7]] tetradeca-1(9),2,5,7-tetraene: Into a 1000-mL round-bottom flask, was placed 3-[(5-bromo-1-[[2-(trimethylsilyl)ethoxy]methyl] pyrrolo[2,3-b]pyridin-6-yl)oxy]propan-1-amine (37.50 g, 93.658 mmol, 1.00 equiv), toluene (500.00 mL), t-BuONa (27.00 g, 280.947 mmol, 3.00 equiv), BrettPhos Pd G3 (4.25 g, 4.688 mmol, 0.05 equiv). The resulting solution was stirred for 4 h at 110° C. The solids were filtered out. The resulting mixture was concentrated. The residue was applied onto a silica gel column with ethyl acetate/petroleum ether (0:1-1:1). This resulted in 16.1 g (53.81%) of 4-[[2-(trimethylsilyl)ethoxy]methyl]-14-oxa-2,4,10-triazatricyclo[7.5.0.0\hat{ }[3,7]]tetradeca-1(9),2,5,7-tetraene as a brown solid. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min.LC-MS: (ES, m/z): M+1:320.

Synthesis of methyl4-(4-[[4-(4-chlorophenyl)-6,6-dimethyl-2,5-dihydropyran-3-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 40-mL vial purged and maintained with an inert atmosphere of nitrogen, was added methyl 2-bromo-4-(4-[[4-(4-chlorophenyl)-6,6-dimethyl-2,5-dihydropyran-3-yl]methyl]piperazin-1-yl)benzoate (500.00 mg, 0.939 mmol, 2.00 equiv), Toluene (5.00 mL), 4-[[2-(trimethylsilyl)ethoxy]methyl]-14-oxa-2,4,10-triazatricyclo[7.5.0.0\hat{ }[3,7]]tetradeca-1(9),2,5,7-tetraene (150.00 mg, 0.47 mmol, 1.00 equiv), Cs₂CO₃ (306.00 mg, 0.94 mmol, 2.00 equiv), Chloro[9,9-dimethyl-4,5-bis(diphenylphosphino)xanthene][2-amino-1,1-biphenyl-2-yl]palladium(II) (41.65 mg, 0.047 mmol, 0.10 equiv). The resulting solution was stirred for overnight at 110° C. The reaction mixture was cooled to RT. The resulting solution was diluted with 10 mL of H₂O. The resulting solution was extracted with 4×10 mL of ethyl acetate and the organic layers combined. The resulting mixture was washed with 20 mL of brine. The mixture was dried over anhydrous sodium sulfate and concentrated. The residue was applied onto a silica gel column with ethyl acetate/petroleum ether (1:5-1:1). This resulted in 250 mg (68.93%) of methyl 4-(4-[[4-(4-chlorophenyl)-6,6-dimethyl-2,5-dihydropyran-3-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as yellow oil. LC-MS: (ES, m/z): M+1: 772.

### Synthesis of 4-(4-[[4-(4-chlorophenyl)-6,6-dimethyl-2,5-dihydropyran-3-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid: Into a 40-mL vial, was added methyl 4-(4-[[4-(4-chlorophenyl)-6,6-dimethyl-2,5-dihydropyran-3-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (250 mg crude, 0.324 mmol, 1.00 equiv), methanol (3 mL), Dioxane (3.00 mL), NaOH(0.48 mL, 1.940 mmol, 6equiv). The resulting solution was stirred for 3 h at 70° C. The reaction mixture was cooled to RT. The resulting solution was diluted with 10 mL of H₂O. HOAc was employed to adjust the pH to 5. The resulting solution was extracted with 4×10 mL of ethyl acetate and the organic layers combined. The resulting mixture was washed with 20×20 mL of brine. The mixture was dried over anhydrous sodium sulfate and concentrated. The residue was applied onto a silica gel column with ethyl acetate/petroleum ether (1:2-2:1). This resulted in 125 mg (50.92%) of 4-(4-[[4-(4-chlorophenyl)-6,6-dimethyl-2,5-dihydropyran-3-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid as brown solid. LC-MS: (ES, m/z): M+1: 758

Synthesis of 4-([[(2S)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as yellow solid. LC-MS: (ES, m/z): M+1:318.

Synthesis of 4-(4-[[4-(4-chlorophenyl)-6,6-dimethyl-2,5-dihydropyran-3-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide: Into a 40-mL vial, was added 4-(4-[[4-(4-chlorophenyl)-6,6-dimethyl-2,5-dihydropyran-3-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (120.00 mg, 0.158 mmol, 1.00 equiv), DCM, 4-[[(2S)-1,4-dioxan-2-ylmethyl]amino]-3-nitrobenzenesulfonamide (60.00 mg, 0.189 mmol, 1.20 equiv), DMAP (38.66 mg, 0.316 mmol, 2.00 equiv), EDCI (36.40 mg, 0.189 mmol, 1.20 equiv). The resulting solution was stirred for 12 h at 30° C. The resulting solution was diluted with 10 mL of H₂O. The resulting solution was extracted with 4×10 mL of ethyl acetate and the organic layers combined. The resulting mixture was washed with 20 mL of brine. The mixture was dried over anhydrous sodium sulfate and concentrated. The residue was applied onto Pre-TLC with dichloromethane/methanol (5:95). This resulted in 100 mg (59.75%) of 4-(4-[[4-(4-chlorophenyl)-6,6-dimethyl-2,5-dihydropyran-3-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0 \hat{ }[3,7]]tetradeca-1(9),2,5,7-tetraen-10-yl)benzamide as yellow solid. LC-MS: (ES, m/z): M+1: 1057.

Synthesis of 4-(4-[[4-(4-chlorophenyl)-6,6-dimethyl-2,5-dihydropyran-3-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide: Into a 40-mL vial, was added 4-(4-[[4-(4-chlorophenyl)-6,6-dimethyl-2,5-dihydropyran-3-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide (100 mg, 0.095 mmol, 1.00 equiv), tetrahydrofuran (4.00 mL), ethylenediamine (113.00 mg, 1.89 mmol, 20 equiv), TBAF (1.89 mL, 1.89 mmol, 20 equiv). The resulting solution was stirred for 8 h at 700° C. The reaction mixture was cooled. The resulting solution was diluted with 10 mL of H₂O. The resulting solution was extracted with 4×10 mL of ethyl acetate and the organic layers combined. The resulting mixture was washed with 20 mL of brine. The mixture was dried over anhydrous sodium sulfate and concentrated. The residue was applied onto Pre-TLC with dichloromethane/methanol (95:5). The residue was applied onto Pre-TLC with dichloromethane/methanol (95:5). The crude product (50 mg) was purified by Flash-Prep-HPLC with column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min. This resulted in 15 mg mg of 4-(4-[[4-(4-chlorophenyl)-6,6-dimethyl-2,5-dihydropyran-3-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0 \hat{ }[3,7]]tetradeca-1(9),2,5,7-tetraen-10-yl]benzamide as a yellow solid. LC-MS: (ES, m/z): M+1:927. ¹H NMR (300 MHz, DMSO-d₆, ppm) δ11.84 (s, 1H), 8.88-8.71 (m, 2H), 8.47 (s, 1H), 7.97 (m, 2H), 7.38 (s, 3H), 7.14 (s, 1H), 7.06 (m, 2H), 6.97 (s, 1H), 6.80-6.63 (m, 3H), 6.22 (s, 1H), 4.58 (s, 4H), 4.04-3.75 (m, 7H), 3.75-3.55 (m, 5H), 3.47 (m, 2H), 3.31 (m, 4H), 2.43 (m, 7H), 1.33 (m, 7H).

### Compound 3-40: Preparation of 4-(4-[[2-(4-chlorophenyl)-5,5-dimethylcyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide

Synthesis of 4-([[(2S)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as a yellow solid. LC-MS: (ES, m/z): M+1=318.

Synthesis of 2-[3-[(5-bromo-1-[[2-(trimethylsilyl)ethoxy]methyl]pyrrolo[2,3-b]pyridin-6-yl)oxy]propyl] isoindole-1,3-dione: Into a 1000-mL 3-necked round-bottom flask purged and maintained with an inert atmosphere of nitrogenn, was placed 5-bromo-6-fluoro-1-[[2-(trimethylsilyl)ethoxy]methyl]pyrrolo [2,3-b]pyridine (53.50 g, 154.944 mmol, 1 equiv). This was followed by the addition of 2-(3-hydroxypropyl)isoindole-1,3-dione (31.80 g, 154.944 mmol, 1 equiv), in portions at degrees C. To this was added Dioxane (500.00 mL) at degrees C., NaH (9.30 g, 232.415 mmol, 1.50 equiv, 60%). The resulting solution was stirred for 4 h at 80 degrees C. The reaction mixture was cooled with a water/ice bath. The reaction was then quenched by the addition of 500 mL AcOH/ice/water. The resulting solution was extracted with 2×500 mL of ethyl acetate. The resulting mixture was washed with 3×500 ml of Brine. The mixture was dried over anhydrous sodium sulfate and concentrated. The residue was applied onto a silica gel column with ethyl acetate/petroleum ether (0:1-1:10). This resulted in 58 g (70.56%) of 2-[3-[(5-bromo-1-[[2-(trimethylsilyl)ethoxy]methyl]pyrrolo [2,3-b]pyridin-6-yl)oxy]propyl]isoindole-1,3-dione as colorless oil. LC-MS: (ES, m/z): M+1=554.

Synthesis of 3-[(5-bromo-1-[[2-(trimethylsilyl)ethoxy]methyl]pyrrolo[2,3-b]pyridin-6-yl)oxy]propan-1-amine: Into a 500-mL round-bottom flask, was placed 2-[3-[(5-bromo-1-[[2-(trimethylsilyl)ethoxy]methyl] pyrrolo[2,3-b]pyridin-6-yl)oxy]propyl]isoindole-1,3-dione (58.00 g, 109.332 mmol, 1.00 equiv), EtOH (300.00 mL), NH₂NH₂.H₂O (68.42 g, 1093.321 mmol, 10 equiv, 80%). The resulting solution was stirred for 4 h at room temperature. The resulting mixture was concentrated. The reaction was then quenched by the addition of 200 mL of water. The resulting solution was extracted with 2×500 mL of ethyl acetate The resulting mixture was washed with 2×300 ml of Brine. The mixture was dried over anhydrous sodium sulfate and concentrated. The residue was applied onto a silica gel column with ethyl acetate/petroleum ether (0:1-1:2). This resulted in 37.5 g (85.66%) of 3-[(5-bromo-1-[[2-(trimethylsilyl)ethoxy]methyl]pyrrolo[2,3-b]pyridin-6-yl)oxy]propan-1-amine as yellow oil.

### Synthesis of 4-[[2-(trimethylsilyl)ethoxy]methyl]-14-oxa-2,4,10-triazatricyclo[7.5.0.0\hat{ }[3,7]] tetradeca-1(9),2,5,7-tetraene: Into a 1000-mL round-bottom flask, was placed 3-[(5-bromo-1-[[2-(trimethylsilyl)ethoxy]methyl] pyrrolo[2,3-b]pyridin-6-yl)oxy]propan-1-amine (37.50 g, 93.658 mmol, 1.00 equiv), toluene (500.00 mL), t-BuONa (27.00 g, 280.947 mmol, 3.00 equiv), BrettPhos Pd G3 (4.25 g, 4.688 mmol, 0.05 equiv). The resulting solution was stirred for 4 h at 110 degrees C. The solids were filtered out. The resulting mixture was concentrated. The residue was applied onto a silica gel column with ethyl acetate/petroleum ether (0:1-1:1). This resulted in 16.1 g (53.81%) of 4-[[2-(trimethylsilyl)ethoxy]methyl]-14-oxa-2,4,10-triazatricyclo [7.5.0.0\hat{ }[3,7]]tetradeca-1(9),2,5,7-tetraene as a brown solid. LC-MS: (ES, m/z): M+1=320

Synthesis of 2-bromo-5,5-dimethylcyclohex-1-ene-1-carbaldehyde Into a 250-mL 3-necked round-bottom flask, was placed DMF (7.24 g, 99.050 mmol, 2.5 equiv), DCM (100 mL). This was followed by the addition of PBr3 (24.67 g, 91.139 mmol, 2.30 equiv) dropwise with stirring at 0 degrees C. The resulting solution was stirred for 1 h at 0 degrees C. To this was added 4,4-dimethylcyclohexan-1-one (5.00 g, 39.620 mmol, 1.00 equiv), in portions at 0 degrees C. The resulting solution was stirred for overnight at room temperature.

The reaction was then quenched by the addition of 100 mL of water/ice. The pH value of the solution was adjusted to 5 with Na₂CO₃. The resulting solution was extracted with 3×100 mL of dichloromethane dried over anhydrous sodium sulfate and concentrated. This resulted in 5 g (58.13%) of 2-bromo-5,5-dimethylcyclohex-1-ene-1-carbaldehyde as yellow oil. LC-MS: (ES, m/z): M+1=217/219.

Synthesis of 2-(4-chlorophenyl)-5,5-dimethylcyclohex-1-ene-1-carbaldehyde Into a 100-mL round-bottom flask, was placed 2-bromo-5,5-dimethylcyclohex-1-ene-1-carbaldehyde (2.50 g, 11.515 mmol, 1.00 equiv), benzeneboronic acid, p-chloro-(1.80 g, 11.515 mmol, 1 equiv), DME (25 mL), H₂O (25 mL), Na₂CO₃ (2.44 g, 23.030 mmol, 2 equiv), Pd(dppf)Cl₂ CH₂Cl2 (0.94 g, 1.151 mmol, 0.10 equiv). The resulting solution was stirred for 12 h at 60 degrees C. The reaction mixture was cooled to room temperature. The reaction was then quenched by the addition of 50 mL of water. The resulting solution was extracted with 3×50 mL of ethyl acetate dried over anhydrous sodium sulfate and concentrated. The residue was applied onto a silica gel column with ethyl acetate/petroleum ether (0:1-1:1). This resulted in 1.2 g (41.89%) of 2-(4-chlorophenyl)-5,5-dimethylcyclohex-1-ene-1-carbaldehyde as yellow oil.LC-MS: (ES, m/z): M+1=249.

### Synthesis of methyl 2-bromo-4-(4-[[2-(4-chlorophenyl)-5,5-dimethylcyclohex-1-en-1-yl]methyl]piperazin-1-yl)benzoate: Into a 100-mL round-bottom flask, was placed 2-(4-chlorophenyl)-5,5-dimethylcyclohex-1-ene-1-carbaldehyde (1.20 g, 4.824 mmol, 1.00 equiv), methyl 2-bromo-4-(piperazin-1-yl)benzoate (1.44 g, 4.824 mmol, 1.00 equiv), DCE (50.00 mL), Ti(Oi-Pr)₄ (4.11 g, 14.472 mmol, 3.00 equiv). The resulting solution was stirred for 3 h at room temperature. This was followed by the addition of NaBH(OAc)₃ (2.04 g, 9.648 mmol, 2.00 equiv), in portions at room temperature. The resulting solution was stirred for overnight at room temperature. The reaction was then quenched by the addition of 10 mL of water. The resulting mixture was concentrated. The residue was applied onto a silica gel column with ethyl acetate/petroleum ether (0:1-1:1). This resulted in 600 mg (23.38%) of methyl 2-bromo-4-(4-[[2-(4-chlorophenyl)-5, 5-dimethylcyclohex-1-en-1-yl]methyl]piperazin-1-yl)benzoate as off-white oil. LC-MS: (ES, m/z): M+1=531/533

Synthesis of methyl 4-(4-[[2-(4-chlorophenyl)-5,5-dimethylcyclohex-1-en-1-yl]methyl]piperazin-1-yl)-2-(4-j[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 40-mL round-bottom flask, was placed 4-[[2-(trimethylsilyl) thoxy]methyl]-14-oxa-2,4,10-triazatricyclo[7.5.0.0\hat{ }[3,7]]tetradeca-1(9),2,5,7-tetraene (150.15 mg, 0.470 mmol, 1.00 equiv), methyl 2-bromo-4-(4-[[2-(4-chlorophenyl)-5,5-dimethylcyclohex-1-en-1-yl]methyl]piper zin-1-yl)benzoate (250.00 mg, 0.470 mmol, 1.00 equiv), Cs₂CO₃ (459.40 mg, 1.410 mmol, 3.00 equiv), Toluene (5.00 mL), xantphos Pd 2G (27.19 mg, 0.047 mmol, 0.10 equiv). The resulting solution was stirred for 12 h at 110 degrees C. The resulting mixture was concentrated. The residue was applied onto a silica gel column with ethyl acetate/petroleum ether (0:1-1:1). This resulted in 120 mg (33.14%) of methyl 4-(4-[[2-(4-chlorophenyl)-5,5-dimethylcyclohex-1-en-1-yl]methyl]piperazin-1-yl)-2-(4-[[2-(trimethylsilyl)ethoxy] ethyl]-14-oxa-2,4,10-triazatricyclo[7.5.0.0\hat{ }[3,7]]tetradeca-1(9),2,5,7-tetraen-10-yl)benzoate as a white solid. LC-MS: (ES, m/z): M+1=770.

Synthesis of 4-(4-[[2-(4-chlorophenyl)-5,5-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid: Into a 8-mL round-bottom flask, was placed methyl 4-(4-[[2-(4-chlorophenyl)-5,5-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (120.00 mg, 0.156 mmol, 1.00 equiv), MeOH (1.00 mL), Dioxane (1.00 mL), H₂O (1.00 mL), NaOH (37.38 mg, 0.934 mmol, 6 equiv). The resulting solution was stirred for 12 h at 70 degrees C. The reaction was then quenched by the addition of 5 mL of water. The pH value of the solution was adjusted to 5 with HCl (1 mol/L). The solids were collected by filtration. This resulted in 100 mg (84.88%) of 4-(4-[[2-(4-chlorophenyl)-5,5-dimethylcyclohex-1-en-1-yl]methyl]piperazin-1-yl)-2-(4-[[2-(trimethylsilyl)ethoxy]methyl]-14-oxa-2,4,10-triazatricyclo [7.5.0.0\hat{ }[3,7]] tetradeca-1(9),2,5,7-tetraen-10-yl)benzoic acid as a white crude solid. LC-MS: (ES, m/z): M+1=756, R.T=1.288 min.

Synthesis of 4-(4-[[2-(4-chlorophenyl)-5,5-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide: Into a 8-mL round-bottom flask, was placed 4-(4-[[2-(4-chlorophenyl)-5,5-dimethylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (100.00 mg, 0.132 mmol, 1.00 equiv), DCM (5 mL), DMAP (64.60 mg, 0.529 mmol, 4 equiv), EDCI (50.68 mg, 0.264 mmol, 2 equiv), 4-[[(2S)-1,4-dioxan-2-ylmethyl]amino]-3-nitrobenzenesulfonamide (46.14 mg, 0.145 mmol, 1.10 equiv). The resulting solution was stirred for overnight at room temperature. The resulting mixture was concentrated. The residue was applied onto a silica gel column with dichloromethane/methanol (1:0-10:1). This resulted in 100 mg (71.65%) of 4-(4-[[2-(4-chlorophenyl)-5,5-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide as a yellow solid. LC-MS: (ES, m/z): M+1=1056.

### Synthesis of 4-(4-[[2-(4-chlorophenyl)-5,5-dimethylcyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0 \hat{ }[3,7]]tetradeca-1(9),2,5,7-tetraen-10-yl]benzamide: Into a 40-mL round-bottom flask, was placed 4-(4-[[2-(4-chlorophenyl)-5,5-dimethylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide (100.00 mg, 0.095 mmol, 1.00 eq), ethylenediamine (113.85 mg, 1.894 mmol, 20.00 eq), TBAF (495.31 mg, 1.894 mmol, 20 eq), THF (10 ml). The resulting solution was stirred for 12 h at 70 degrees C. The reaction was then quenched by the addition of 10 mL of water. The resulting solution was extracted with 2×10 mL of ethyl acetate concentrated. The crude product was purified by Prep-HPLC with the following conditions (Waters-2767): Column, X-bridge RP18, Sum, 19*100 mm; mobile phase, 0.03% ammonia in water (0.03% NH₄HCO₃ &NH₄OH) and CH₃CN (32% CH₃CN up to 52% in 6 min); Detector, UV 254 nm. This resulted in 25 mg (28.52%) of 4-(4-[[2-(4-chlorophenyl)-5,5-dimethylcyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide as a yellow solid. LC-MS: (ES, m/z): M+1=925. ¹H-NMR: (DMSO-d₆,300 ppm) δ: 11.91 (s, 1H), 11.22 (s, 1H), 8.52-8.48 (m, 2H), 7.68-7.63 (m, 1H), 7.47-7.45 (d, J=6 Hz, 1H), 7.38-7.35 (d, J=9 Hz, 2H), 7.20 (s, 1H), 7.12-7.09 (d, J=9 Hz, 2H), 6.92-6.89 (m, 2H), 6.72 (s, 1H), 6.13-6.11 (m, 1H), 4.21 (s, 2H), 3.82-3.75 (m, 3H), 3.68-3.51 (m, 8H), 3.22-3.19 (m, 2H), 2.73 (s, 2H), 2.3-2.10 (m, 4H), 2.08-1.90 (m, 3H), 1.40 (m, 2H), 1.21 (m, 1H) 0.97 (s, 6H)

### Compound 3-41. Preparation of 4-(4-[[2-(4-chlorophenyl)cyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide

Synthesis of 2-bromocyclohex-1-ene-1-carbaldehyde: Into a 250-mL 3-necked round-bottom flask, was added CHCl₃ (80.00 mL, 991.80 mmol, 19.47 equiv). This was followed by the addition of DMF (9.31 g, 0.12 mmol, 2.5 equiv) dropwise with stirring at 0° C. To this was added PBr₃ (31.72 g, 0.11 mmol, 2.3 equiv) dropwise with stirring at 0° C. The resulting solution was stirred for 1 h at 0° C. To the mixture was added cyclohexanone (5.00 g, 50.94 mmol, 1.00 equiv) dropwise with stirring at RT. The resulting solution was allowed to react for overnight at RT. The reaction was then quenched by the addition of 100 mL of water/ice. The pH value of the solution was adjusted to 5 with Na₂CO₃. The resulting solution was extracted with 3×100 mL of dichloromethane dried over anhydrous sodium sulfate and concentrated. This resulted in 5.2 g (53.99%) of 2-bromocyclohex-1-ene-1-carbaldehyde as light yellow oil. LC-MS: (ES, m/z): M+1: 189.

Synthesis of 2-(4-chlorophenyl)cyclohex-1-ene-1-carbaldehyde: Into a 100-mL round-bottom flask, was added 2-bromocyclohex-1-ene-1-carbaldehyde (5.00 g, 26.44 mmol, 1.00 equiv), DME (50.00 mL), p-chloro-benzeneboronic acid (4.14 g, 26.44 mmol, 1 equiv), Na₂CO₃(5.66 g, 52.89 mmol, 2.0 equiv), Pd₂(dba)₃ (2.42 g, 2.64 mmol, 1 equiv), water(5 mL). The resulting solution was stirred for 12 h at 60° C. The reaction mixture was cooled to RT. The resulting solution was diluted with 50 mL of H₂O. The resulting solution was extracted with 3×50 mL of ethyl acetate. The resulting mixture was washed with 50 mL of brine. The mixture was dried over anhydrous sodium sulfate and concentrated. The residue was applied onto a silica gel column with ethyl acetate/petroleum ether (1:10-2:5). This resulted in 3.5 g (59.96%) of 2-(4-chlorophenyl) cyclohex-1-ene-1-carbaldehyde as light yellow oil. LC-MS: (ES, m/z): M+1: 221.

Synthesis of methyl 2-bromo-4-(4-[[2-(4-chlorophenyl)cyclohex-1-en-1-yl]methyl]piperazin-1-yl)benzoate: Into a 100-mL 3-necked round-bottom flask, was added 2-(4-chlorophenyl) cyclohex-1-ene-1-carbaldehyde (3.50 g, 15.85 mmol, 1.00 equiv), DCE (35.00 mL, 442.10 mmol, 27.88 equiv), methyl 2-bromo-4-(piperazin-1-yl) benzoate (4.74 g, 15.85 mmol, 1 equiv), Ti(Oi-Pr)₄ (13.52 g, 47.57 mmol, 3 equiv). The resulting solution was stirred for 3 h at RT. This was followed by the addition of NaBH(AcO)₃ (6.72 g, 31.71 mmol, 2 equiv) in several batches at RT. The resulting solution was allowed to react for an additional 16 h at RT. The reaction was then quenched by the addition of 10 mL of MeOH. The resulting mixture was concentrated. The residue was applied onto a silica gel column with ethyl acetate/petroleum ether (1:10-2:5). This resulted in 4.2 g (52.56%) of methyl 2-bromo-4-(4-[[2-(4-chlorophenyl) cyclohex-1-en-1-yl]methyl]piperazin-1-yl)benzoate as light yellow oil. LC-MS: (ES, m/z): M+1: 503.

Synthesis of 2-[3-[(5-bromo-1-[[2-(trimethylsilyl)ethoxy]methyl]pyrrolo[2,3-b]pyridine-6-yl)oxy] propyl]isoindole-1,3-dione: Into a 1000-mL 3-necked round-bottom flask purged and maintained with an inert atmosphere of nitrogen, was placed 5-bromo-6-fluoro-1-[[2-(trimethylsilyl)ethoxy]methyl]pyrrolo[2,3-b]pyridine (53.50 g, 154.944 mmol, 1 equiv). This was followed by the addition of 2-(3-hydroxypropyl) isoindole-1,3-dione (31.80 g, 154.944 mmol, 1 equiv), in portions at RT. To this was added Dioxane (500.00 mL) at RT, NaH (9.30 g, 232.415 mmol, 1.50 equiv, 60%). The resulting solution was stirred for 4 h at 80° C. The reaction mixture was cooled with a water/ice bath. The reaction was then quenched by the addition of 500 mL AcOH/ice/water. The resulting solution was extracted with 2×500 mL of ethyl acetate. The resulting mixture was washed with 3×500 ml of Brine. The mixture was dried over anhydrous sodium sulfate and concentrated. The residue was applied onto a silica gel column with ethyl acetate/petroleum ether (0:1-1:10). This resulted in 58 g (70.56%) of 2-[3-[(5-bromo-1-[[2-(trimethylsilyl)ethoxy]methyl]pyrrolo[2,3-b]pyridin-6-yl)oxy]propyl]isoindole-1,3-dione as colorless oil. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min.LC-MS: (ES, m/z): M+1:554.

Synthesis of 3-[(5-bromo-1-[[2-(trimethylsilyl)ethoxy]methyl]pyrrolo[2,3-b]pyridin-6-yl)oxy]propan-1-amine: Into a 500-mL round-bottom flask, was placed ethoxy]methyl] propyl]isoindole-pyrrolo[2,3-b]pyridin-6-yl)oxy]2-[3-[(5-bromo-1-[[2-(trimethylsilyl)1,3-dione(58.00 g,109.33 mmol,1.0equiv), EtOH (300.00 mL), NH₂NH₂.H₂O (68.42 g, 1093.32 mmol, 10 equiv, 80%). The resulting solution was stirred for 4 h at RT. The resulting mixture was concentrated. The reaction was then quenched by the addition of 200 mL of water. The resulting solution was extracted with 2×500 mL of ethyl acetate. The resulting mixture was washed with 2×300 ml of Brine. The mixture was dried over anhydrous sodium sulfate and concentrated. The residue was applied onto a silica gel column with ethyl acetate/petroleum ether (0:1-1:2). This resulted in 37.5 g(85.66%) of 3-[(5-bromo-1-[[2-(trimethylsilyl)ethoxy]methyl]pyrrolo[2,3-b]pyridin-6-yl)oxy] propan-1-amine as yellow oil.

Synthesis of 4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 1000-mL round-bottom flask, was placed 3-[(5-bromo-1-[[2-(trimethylsilyl)ethoxy]methyl] pyrrolo[2,3-b]pyridin-6-yl)oxy]propan-1-amine (37.50 g, 93.658 mmol, 1.00 equiv), toluene (500.00 mL), t-BuONa (27.00 g, 280.947 mmol,3.00 equiv), BrettPhos Pd G3 (4.25 g, 4.688 mmol, 0.05 equiv). The resulting solution was stirred for 4 h at 110 degrees C. The solids were filtered out. The resulting mixture was concentrated. The residue was applied onto a silica gel column with ethyl acetate/petroleum ether (0:1-1:1). This resulted in 16.1 g (53.81%) of 4-[[2-(trimethylsilyl)ethoxy]methyl]-14-oxa-2,4,10-triazatricyclo[7.5.0.0\hat{ }[3,7]]tetradeca-1(9),2,5,7-tetraene as a brown solid. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min.LC-MS: (ES, m/z): M+1:320.

### Synthesis of methyl 4-(4-[[2-(4-chlorophenyl)cyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 40-mL flask purged and maintained with an inert atmosphere of nitrogen, was added methyl 2-bromo-4-(4-[[2-(4-chlorophenyl)cyclohex-1-en-1-yl]methyl]piperazin-1-yl)benzoate (473.8 mg, 2 equiv), toluene (5 mL), 4-[[2-(trimethylsilyl)ethoxy]methyl]-14-oxa-2,4,10-triazatricyclo[7.5.0.0\hat{ }[3,7]]tetradeca-1(9),2,5,7-tetraene (150 mg, 1.00 equiv), Cs₂CO₃(306.4 mg, 2 equiv), X-antphos G2 (50 mg). The resulting solution was stirred for 12 h at 110° C. The resulting solution was diluted with 10 mL of H₂O. The resulting solution was extracted with 4×10 mL of ethyl acetate. The resulting mixture was washed with 20 mL of brine. The mixture was dried over anhydrous sodium sulfate and concentrated. The residue was applied onto a silica gel column with ethyl acetate/petroleum ether (1:5-1:1). This resulted in 280 mg (80.33%) of methyl 4-(4-[[2-(4-chlorophenyl)cyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as light yellow oil. LC-MS: (ES, m/z): M+1: 742

Synthesis of 4-(4-[[2-(4-chlorophenyl)cyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid: Into a 40-mL round-bottom flask, was added methyl 4-(4-[[2-(4-chlorophenyl)cyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (280.00 mg, 0.37 mmol, 1.00 equiv), methanol (3.00 mL, 0.094 mmol, 0.25 equiv), dioxane (3.00 mL, 35.41 mmol, 93.90 equiv), NaOH (0.57 mL, 2.262 mmol, 6.00 equiv). The resulting solution was stirred for 3 h at 70° C. The reaction mixture was cooled to RT. The resulting solution was diluted with 10 mL of H₂O. The pH value of the solution was adjusted to 5 with HOAc. The resulting solution was extracted with 4×10 mL of ethyl acetate. The resulting mixture was washed with 20 mL of brine. The mixture was dried over anhydrous sodium sulfate and concentrated. The residue was applied onto a silica gel column with ethyl acetate/petroleum ether (1:2-2:1). This resulted in 100 mg (36.40%) of 4-(4-[[2-(4-chlorophenyl)cyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid as a brown solid. LC-MS: (ES, m/z): M+1: 728.

Synthesis of 4-([[(2S)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as yellow solid. LC-MS: (ES, m/z): M+1:318.

Synthesis of 4-(4-[[2-(4-chlorophenyl)cyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide: Into a 40-mL round-bottom flask, was added 4-(4-[[2-(4-chlorophenyl)cyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (95.00 mg, 0.130 mmol, 1.00 equiv), DCM (2.00 mL), DMAP (31.87 mg, 0.261 mmol, 2 equiv), 4-[[(2S)-1,4-dioxan-2-ylmethyl]amino]-3-nitrobenzenesulfonamide (49.60 mg, 0.156 mmol, 1.20 equiv), EDCI (30.00 mg, 0.15 mmol, 1.2 equiv). The resulting solution was stirred for 12 h at 30° C. The resulting solution was diluted with 10 mL of H₂O. The resulting solution was extracted with 4×10 mL of ethyl acetate and the organic layers combined. The resulting mixture was washed with 20 mL of brine. The mixture was dried over anhydrous sodium sulfate and concentrated. The residue was applied onto a silica gel column with dichloromethane/methanol (95:5). This resulted in 80 mg (59.69%) of 4-(4-[[2-(4-chlorophenyl)cyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide as yellow solid. LC-MS: (ES, m/z): M+1: 1027.

Synthesis of 4-(4-[[2-(4-chlorophenyl)cyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide: Into a 40-mL round-bottom flask, was added 4-(4-[[2-(4-chlorophenyl)cyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide (80.00 mg, 0.078 mmol, 1.00 equiv), THE (4 mL), ethylenediamine (93.57 mg, 1.55 mmol, 20 equiv), TBAF (1.55 g, 5.92 mmol, 76.16 equiv). The resulting solution was stirred for 8 h at 70° C. The reaction mixture was cooled to RT. The resulting solution was diluted with 10 mL of H₂O. The resulting solution was extracted with 4×10 mL of ethyl acetate and the organic layers combined. The resulting mixture was washed with 20 mL of brine. The mixture was dried over anhydrous sodium sulfate and concentrated. The residue was applied onto a silica gel column with dichloromethane/methanol (95:5). The crude product (35 mg) was purified by Prep-HPLC with the following conditions: column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min. This resulted in 19 mg (27.20%) of 4-(4-[[2-(4-chlorophenyl)cyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide as yellow solid. LC-MS: (ES, m/z): M+1: 897, ¹H-NMR: (300 MHz, DMSO-d₆, ppm) δ11.94 (s, 1H), 8.88-8.60 (m, 2H), 8.45 (s, 1H), 8.02 (m, 1H), 7.95 (m, 1H), 7.13 (m, 1H), 7.09-6.93 (m, 3H), 6.74 (s, 2H), 6.20 (s, 1H), 4.13-3.56 (m, 8H), 3.60-3.39 (m, 2H), 3.31 (s, 6H), 2.84 (s, 2H), 2.38 (s, 5H), 2.19 (d, J=30.5 Hz, 5H), 1.73 (s, 5H).

### Compound 3-42: Preparation of 4-(4-[[2-(4-chlorophenyl)cyclohept-1-en-1-yl]methyl]piperazin-1-yl)-N-(4-[[(2S)-1,4-dioxan-2-ylmethyl]amino]-3-nitro benzenesulfonyl)-2-[14-oxa-2,4,10-triazatricyclo [7.5.0.0\hat{ }[3,7]]tetradeca-1(9),2,5,7-tetraen-10-yl]benzamide

Synthesis of 4-([[(2S)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as a yellow solid. LC-MS: (ES, m/z): M+1=554.

Synthesis of 2-[3-[(5-bromo-1-[[2-(trimethylsilyl)ethoxy]methyl]pyrrolo[2,3-b]pyridin-6-yl)oxy] propyl]isoindole-1,3-dione: Into a 1000-mL 3-necked round-bottom flask purged and maintained with an inert atmosphere of nitro gen, was placed 5-bromo-6-fluoro-1-[[2-(trimethylsilyl)ethoxy]methyl]pyrrolo[2,3-b]pyridine (53.50 g, 154.944 mmol, 1 equiv). This was followed by the addition of 2-(3-hydroxypropyl)isoindole-1,3-dione (31.80 g, 154.944 mmol, 1 equiv), in portions at 0 degrees C. To this was added Dioxane (500.00 mL) at degrees C., NaH (9.30 g, 232.415 mmol, 1.50 equiv, 60%). The resulting solution was stirred for 4 h at 80 degrees C. The reaction mixture was cooled with a water/ice bath. The reaction was then quenched by the addition of 500 mL AcOH/ice/water. The resulting solution was extracted with 2×500 mL of ethyl acetate. The resulting mixture was washed with 3×500 ml of Brine. The mixture was dried over anhydrous sodium sulfate and concentrated. The residue was applied onto a silica gel column with ethyl acetate/petroleum ether (0:1-1:10). This resulted in 58 g (70.56%) of 2-[3-[(5-bromo-1-[[2-(trimethylsilyl)ethoxy]methyl]pyrrolo [2,3-b]pyridin-6-yl)oxy]propyl]isoindole-1,3-dione as colorless oil. LC-MS: (ES, m/z): M+1=554.

Synthesis of 3-[(5-bromo-1-[[2-(trimethylsilyl)ethoxy]methyl]pyrrolo[2,3-b]pyridin-6-yl)oxy] propan-1-amine Into a 500-mL round-bottom flask, was placed 2-[3-[(5-bromo-1-[[2-(trimethylsilyl)ethoxy]methyl] pyrrolo[2,3-b]pyridin-6-yl)oxy]propyl]isoindole-1,3-dione (58.00 g, 109.332 mmol, 1.00 equiv), EtOH (300.00 mL), NH₂NH₂.H₂O (68.42 g, 1093.321 mmol, 10 equiv, 80%). The resulting solution was stirred for 4 h at room temperature. The resulting mixture was concentrated. The reaction was then quenched by the addition of 200 mL of water. The resulting solution was extracted with 2×500 mL of ethyl acetate. The resulting mixture was washed with 2×300 ml of Brine. The mixture was dried over anhydrous sodium sulfate and concentrated. The residue was applied onto a silica gel column with ethyl acetate/petroleum ether (0:1-1:2). This resulted in 37.5 g (85.66%) of 3-[(5-bromo-1-[[2-(trimethylsilyl)ethoxy]methyl]pyrrolo[2,3-b]pyridin-6-yl)oxy]propan-1-amine as yellow oil.

Synthesis of 4-[[2-(trimethylsilyl)ethoxy]methyl]-14-oxa-2,4,10-triazatricyclo[7.5.0.0\hat{ }[3,7]] tetradeca-1(9),2,5,7-tetraene: Into a 1000-mL round-bottom flask, was placed 3-[(5-bromo-1-[[2-(trimethylsilyl)ethoxy]methyl] pyrrolo[2,3-b]pyridin-6-yl)oxy]propan-1-amine (37.50 g, 93.658 mmol, 1.00 equiv), toluene (500.00 mL), t-BuONa (27.00 g, 280.947 mmol, 3.00 equiv), BrettPhos Pd G3 (4.25 g, 4.688 mmol, 0.05 equiv). The resulting solution was stirred for 4 h at 110 degrees C. The solids were filtered out. The resulting mixture was concentrated. The residue was applied onto a silica gel column with ethyl acetate/petroleum ether (0:1-1:1). This resulted in 16.1 g (53.81%) of 4-[[2-(trimethylsilyl)ethoxy]methyl]-14-oxa-2,4,10-triazatricyclo[7.5.0.0\hat{ }[3,7]]tetradeca-1(9),2,5,7-tetraene as a brown solid. LC-MS: (ES, m/z): M+1=320.

Synthesis of 2-bromocyclohept-1-ene-1-carbaldehyde: Into a 250-mL 3-necked round-bottom flask, was placed DMF (8.15 g, 111.436 mmol, 2.5 equiv), DCM (100.00 mL). This was followed by the addition of PBr₃ (27.75 g, 102.521 mmol, 2.3 equiv) dropwise with stirring at 0 degrees C. The resulting solution was stirred for 1 h at 0 degrees C. To this was added cycloheptanone (5.00 g, 44.574 mmol, 1.00 equiv), in portions at degrees C. The resulting solution was stirred for overnight at room temperature. The reaction was then quenched by the addition of 100 mL of water/ice. The pH value of the solution was adjusted to 5 with Na₂CO₃. The resulting solution was extracted with 3×100 mL of dichloromethane dried over anhydrous sodium sulfate and concentrated. This resulted in 5 g (55.24%) of 2-bromocyclohept-1-ene-1-carbaldehyde as yellow oil. LC-MS: (ES, m/z): M+1=203/205.

Synthesis of 2-(4-chlorophenyl)cyclohept-1-ene-1-carbaldehyde: Into a 100-mL round-bottom flask, was placed 2-bromocyclohept-1-ene-1-carbaldehyde (2.50 g, 12.310 mmol, 1.00 equiv), (4-chlorophenyl)boronic acid (1.92 g, 12.279 mmol, 1.00 equiv), DME (25.00 mL), H₂O (25.00 mL), Na₂CO₃ (2.61 g, 24.621 mmol, 2 equiv), Pd(pph₃)₂Cl₂ (1.01 g, 1.231 mmol, 0.1 equiv). The resulting solution was stirred for 12 h at 60 degrees C. The reaction mixture was cooled to room temperature. The reaction was then quenched by the addition of 50 mL of water. The resulting solution was extracted with 3×50 mL of ethyl acetate. The resulting mixture was washed with 3×50 ml of Brine. The mixture was dried over anhydrous sodium sulfate and concentrated. The residue was applied onto a silica gel column with ethyl acetate/petroleum ether (0:1-1:1). This resulted in 0.6 g (20.76%) of 2-(4-chlorophenyl)cyclohept-1-ene-1-carbaldehyde as yellow oil.LC-MS: (ES, m/z): M+1=235.

Synthesis of methyl 2-bromo-4-(4-[[2-(4-chlorophenyl)cyclohept-1-en-1-yl]methyl]piperazin-1-yl) benzoate: Into a 100-mL round-bottom flask, was placed 2-(4-chlorophenyl)cyclohept-1-ene-1-carbaldehyde (0.60 g, 2.556 mmol, 1.00 equiv), methyl 2-bromo-4-(piperazin-1-yl)benzoate (0.76 g, 2.540 mmol, 0.99 equiv), DCM (30.00 mL, 471.901 mmol, 184.61 equiv), Ti(Oi-Pr)₄ (2.18 g, 7.669 mmol, 3.00 equiv). This was followed by the addition of NaBH(OAc)₃ (1.08 g, 5.112 mmol, 2.00 equiv), in portions at room temperature. The resulting solution was stirred for overnight at room temperature. The reaction was then quenched by the addition of 10 mL of water. The resulting mixture was concentrated. The residue was applied onto a silica gel column with ethyl acetate/petroleum ether (0:1-1:1). This resulted in 500 mg (37.77%) of methyl 2-bromo-4-(4-[[2-(4-chlorophenyl)cyclohept-1-en-1-yl]methyl]piperazin-1-yl)benzoate as off-white oil. LC-MS: (ES, m/z): M+1=517/519.

Synthesis of methyl 4-(4-[[2-(4-chlorophenyl)cyclohept-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 40-mL round-bottom flask, was placed 4-[[2-(trimethylsilyl)ethoxy]methyl]-14-oxa-2,4,10-triazatricyclo[7.5.0.0\hat{ }[3,7]]tetradeca-1(9),2,5,7-tetraene (154.22 mg, 0.483 mmol, 1.00 equiv), methyl 2-bromo-4-(4-[[2-(4-chlorophenyl)cyclohept-1-en-1-yl]methyl]piperazin-1-yl)benzoate (250.00 mg, 0.483 mmol, 1.00 equiv), Cs₂CO₃ (471.85 mg, 1.448 mmol, 3 equiv), Toluene (5.00 mL), Xantphos Pd 2G (46.00 mg, 0.1 equiv). The resulting solution was stirred for 11 h at 110 degrees C. The resulting mixture was concentrated. The residue was applied onto a silica gel column with ethyl acetate/petroleum ether (0:1-1:1). This resulted in 110 mg (30.12%) of methyl 4-(4-[[2-(4-chlorophenyl)cyclohept-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as a solid. LC-MS: (ES, m/z): M+1=756.

Synthesis of 4-(4-[[2-(4-chlorophenyl)cyclohept-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl) benzoic acid. Into a 8-mL round-bottom flask, was placed methyl 4-(4-[[2-(4-chlorophenyl)cyclohept-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (110.00 mg, 0.145 mmol, 1.00 equiv), MeOH (1.00 mL), Dioxane (1.00 mL), H₂O (1.00 mL, 55.508 mmol, 381.73 equiv), NaOH (34.90 mg, 0.873 mmol, 6.00 equiv). The resulting solution was stirred for 12 hr at 70 degrees C. The reaction was then quenched by the addition of 5 mL of water. The pH value of the solution was adjusted to 5 with HCl (1 mol/L). The solids were collected by filtration. This resulted in 100 mg (92.63%) of 4-(4-[[2-(4-chlorophenyl)cyclohept-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl)benzoic acid as a white crude solid. LC-MS: (ES, m/z): M+1=742.

Synthesis of 4-(4-[[2-(4-chlorophenyl)cyclohept-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4, 10-triazatricyclo[7.5.0.0\hat{ }[3,7]]tetradeca-1(9),2,5,7-tetraen-10-yl)benzamide. Into a 8-mL round-bottom flask, was placed 4-(4-[[2-(4-chlorophenyl)cyclohept-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl)benzoic acid (100.00 mg, 0.135 mmol, 1.00 equiv), DCM (5.00 mL), DMAP (65.82 mg, 0.539 mmol, 4 equiv), EDCI (51.64 mg, 0.269 mmol, 2 equiv), 4-[[(2S)-1,4-dioxan-2-ylmethyl]amino]-3-nitrobenzenesulfonamide (47.01 mg, 0.148 mmol, 1.1 equiv). The resulting solution was stirred for overnight at room temperature. The resulting mixture was concentrated. The residue was applied onto a silica gel column with dichloromethane/methanol (1:0-10:1). This resulted in 120 mg (85.52%) of 4-(4-[[2-(4-chlorophenyl)cyclohept-1-en-1-yl]methyl]piperazin-1-yl)-N-(4-[[(2S)-1,4-dioxan-2-ylmethyl] amino]-3-nitrobenzenesulfonyl)-2-(4-[[2-(trimethylsilyl)ethoxy]methyl]-14-oxa-2,4,10-triazatricyclo[7.5.0.0\hat{ }[3,7]] tetradeca-1(9),2,5,7-tetraen-10-yl)benzamide as a yellow crude solid. LC-MS: (ES, m/z): M+1=1041.

Synthesis of 4-(4-[[2-(4-chlorophenyl)cyclohept-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo [7.5.0.0\hat{ }[3,7]]tetrad eca-1(9),2,5,7-tetraen-10-yl]benzamide. Into a 40-mL round-bottom flask, was placed 4-(4-[[2-(4-chlorophenyl)cyclohept-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy] methyl]-14-oxa-2,4,10-triazatricyclo[7.5.0.0\hat{ }[3,7]] tetradeca-1(9),2,5,7-tetraen-10-yl)benzamide (120.00 mg, 0.115 mmol, 1.00 equiv), ethylenediamine (138.46 mg, 2.304 mmol, 20.00 equiv), THE (10 mL), TBAF (602.37 mg, 2.304 mmol, 20 equiv). The resulting solution was stirred for 12 hr at 70 degrees C. The reaction was then quenched by the addition of 10 mL of water. The resulting solution was extracted with 2×10 mL of ethyl acetate concentrated. The crude product was purified by Prep-HPLC with the following conditions (Waters-2767): Column, X-bridge RP18, 5 um, 19*100 mm; mobile phase, 0.03% ammonia in water (0.03% NH₄HCO₃ & NH₄OH) and CH₃CN (32% CH₃CN up to 52% in 6 min); Detector, UV 254 nm. This resulted in 26 mg (24.76%) of 4-(4-[[2-(4-chlorophenyl)cyclohept-1-en-1-yl]methyl]piperazin-1-yl)-N-(4-[[(2S)-1,4-dioxan-2-ylmethyl]amino]-3-nitro benzenesulfonyl)-2-[14-oxa-2,4,10-triazatricyclo [7.5.0.0\hat{ }[3,7]]tetradeca-1(9),2,5,7-tetraen-10-yl]benzamide as a solid. LC-MS: (ES, m/z): M+1=911. ¹H NMR (300 MHz, DMSO-d₆) δ 11.90 (s, 1H), 11.20 (s, 1H), 8.46 (s, 2H), 7.65 (dd, J=9.1, 1.8 Hz, 2H), 7.45 (s, 1H), 7.36 (d, J=8.3 Hz, 4H), 7.20 (s, 2H), 7.08 (d, J=8.4 Hz, 4H), 6.97-6.86 (m, 4H), 6.70 (s, 2H), 6.12 (d, J=2.5 Hz, 2H), 4.21 (s, 3H), 3.86-3.72 (m, 6H), 3.71-3.41 (m, 13H), 3.20 (s, 4H), 2.76 (s, 4H), 2.39 (s, 7H), 2.28 (s, 7H), 1.98 (s, 2H), 1.57 (s, 3H), 1.50 (s, 3H), 1.24 (s, 1H).

### Compound 3-43: Preparation of 4-(4-[[2-(4-chlorophenyl)-5-methoxy-5-methylcyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0 \hat{ }[3,7]]tetradeca-1(9),2,5,7-tetraen-10-yl]benzamide

### Synthesis of methyl 2-bromo-4-(4-[[2-(4-chlorophenyl)-5-methoxy-5-methylcyclohex-1-en-1-yl]methyl]piperazin-1-yl)benzoate: Into a 100-mL 3-necked round-bottom flask, was added methyl 2-bromo-4-(piperazin-1-yl)benzoate (2.00 g, 6.68 mmol, 1.00 equiv), DCE (20 mL), 2-(4-chlorophenyl)-5-methoxy-5-methylcyclohex-1-ene-1-carbaldehyde (1.77 g, 6.685 mmol, 1 equiv), Ti(Oi-Pr)₄ (5.70 g, 20.05 mmol, 3.00 equiv). The resulting solution was stirred for 3 h at RT. This was followed by the addition of NaBH(OAc)₃ (2.83 g, 13.37 mmol, 2.00 equiv) in several batches at RT. The resulting solution was allowed to react, for an additional 16 h at Rt. The reaction was then quenched by the addition of 10 mL of water. The resulting mixture was concentrated. The residue was applied onto a silica gel column with ethyl acetate/petroleum ether (1:10-2:5). This resulted in 3.0 g (81.90%) of methyl 2-bromo-4-(4-[[2-(4-chlorophenyl)-5-methoxy-5-methylcyclohex-1-en-1-yl]methyl]piperazin-1-yl)benzoate as light yellow oil. LC-MS: (ES, m/z): 547 [M+H]⁺

Synthesis of 2-[3-[(5-bromo-1-[[2-(trimethylsilyl)ethoxy]methyl]pyrrolo[2,3-b]pyridine-6-yl)oxy] propyl]isoindole-1,3-dione: Into a 1000-mL 3-necked round-bottom flask purged and maintained with an inert atmosphere of nitrogen, was placed 5-bromo-6-fluoro-1-[[2-(trimethylsilyl)ethoxy]methyl]pyrrolo[2,3-b]pyridine (53.50 g, 154.944 mmol, 1 equiv). This was followed by the addition of 2-(3-hydroxypropyl)isoindole-1,3-dione (31.80 g, 154.944 mmol, 1 equiv), in portions at RT. To this was added Dioxane (500.00 mL) at RT, NaH (9.30 g, 232.415 mmol, 1.50 equiv, 60%). The resulting solution was stirred for 4 h at 80° C. The reaction mixture was cooled with a water/ice bath. The reaction was then quenched by the addition of 500 mL AcOH/ice/water. The resulting solution was extracted with 2×500 mL of ethyl acetate. The resulting mixture was washed with 3×500 ml of Brine.

The mixture was dried over anhydrous sodium sulfate and concentrated. The residue was applied onto a silica gel column with ethyl acetate/petroleum ether (0:1-1:10). This resulted in 58 g (70.56%) of 2-[3-[(5-bromo-1-[[2-(trimethylsilyl)ethoxy]methyl]pyrrolo[2,3-b]pyridin-6-yl)oxy]propyl]isoindole-1,3-dione as colorless oil. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min.LC-MS: (ES, m/z): M+1:554.

Synthesis of 3-[(5-bromo-1-[[2-(trimethylsilyl)ethoxy]methyl]pyrrolo[2,3-b]pyridin-6-yl)oxy]propan-1-amine: Into a 500-mL round-bottom flask, was placed ethoxy]methyl] propyl]isoindole-pyrrolo[2,3-b]pyridin-6-yl)oxy]2-[3-[(5-bromo-1-[[2-(trimethylsilyl)1,3-dione(58.00 g,109.33 mmol,1.0equiv), EtOH (300.00 mL), NH₂NH₂.H₂O (68.42 g, 1093.32 mmol, 10 equiv, 80%). The resulting solution was stirred for 4 h at RT. The resulting mixture was concentrated. The reaction was then quenched by the addition of 200 mL of water. The resulting solution was extracted with 2×500 mL of ethyl acetate The resulting mixture was washed with 2×300 ml of Brine. The mixture was dried over anhydrous sodium sulfate and concentrated. The residue was applied onto a silica gel column with ethyl acetate/petroleum ether (0:1-1:2). This resulted in 37.5 g (85.66%) of 3-[(5-bromo-1-[[2-(trimethylsilyl)ethoxy]methyl]pyrrolo[2,3-b]pyridin-6-yl)oxy] propan-1-amine as yellow oil.

Synthesis of 4-[[2-(trimethylsilyl)ethoxy]methyl]-14-oxa-2,4,10-triazatricyclo[7.5.0.0\hat{ }[3,7]]tetradeca-1(9),2,5,7-tetraene: Into a 1000-mL round-bottom flask, was placed 3-[(5-bromo-1-[[2-(trimethylsilyl)ethoxy]methyl] pyrrolo[2,3-b]pyridin-6-yl)oxy]propan-1-amine (37.50 g, 93.658 mmol, 1.00 equiv), toluene (500.00 mL), t-BuONa (27.00 g, 280.947 mmol, 3.00 equiv), BrettPhos Pd G3 (4.25 g, 4.688 mmol, 0.05 equiv). The resulting solution was stirred for 4 h at 110 degrees C. The solids were filtered out. The resulting mixture was concentrated. The residue was applied onto a silica gel column with ethyl acetate/petroleum ether (0:1-1:1). This resulted in 16.1 g (53.81%) of 4-[[2-(trimethylsilyl)ethoxy]methyl]-14-oxa-2,4,10-triazatricyclo[7.5.0.0\hat{ }[3,7]]tetradeca-1(9),2,5,7-tetraene as a brown solid. The measurements of the retention were done with a reversed phase column (C18). Shimadzu LCMS 2020; 50*3.0 SUPELCO Ascentis Express C18, 2.7 um; Eluent A: water (0.05% TFA); Eluent B: Acetonitrile; linear gradient from 5% acetonitrile to 95% acetonitrile in 7.0 minutes; Oven temperature 4° C.; flow: 1.5 mL/min.LC-MS: (ES, m/z): M+1:320.

Synthesis of methyl 4-(4-[[2-(4-chlorophenyl)-5-methoxy-5-methylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate: Into a 40-mL round-bottom flask purged and maintained with an inert atmosphere of nitrogen, was added methyl 2-bromo-4-(4-[[2-(4-chlorophenyl)-5-methoxy-5-methylcyclohex-1-en-1-yl]methyl]piperazin-1-yl)benzoate (515 mg, 0.940 mmol,1.00 equiv), Toluene (5 mg), 4-[[2-(trimethylsilyl)ethoxy]methyl]-14-oxa-2,4,10-triazatricyclo[7.5.0.0\hat{ }[3,7]]tetradeca-1(9),2,5,7-tetraene (150.14 mg, 0.47 mmol, 0.50 equiv), Cs₂CO₃ (306.24 mg, 0.94 mmol, 1.00 equiv), X-antphos Pd G3 (50.00 mg, 0.05 mmol, 0.06 equiv). The resulting solution was stirred for 12 h at 110° C. The resulting solution was diluted with 10 mL of H₂O. The resulting solution was extracted with 4×10 mL of ethyl acetate and the organic layers combined. The resulting mixture was washed with 20 mL of brine. The mixture was dried over anhydrous sodium sulfate and concentrated. The residue was applied onto a silica gel column with ethyl acetate/petroleum ether (1:5-1:1). This resulted in 230 mg (62.29%) of methyl 4-(4-[[2-(4-chlorophenyl)-5-methoxy-5-methylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate as light yellow oil. LC-MS: (ES, m/z): 786 [M+H]⁺.

Synthesis of 4-(4-[[2-(4-chlorophenyl)-5-methoxy-5-methylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid: Into a 40-mL vial, was added methyl 4-(4-[[2-(4-chlorophenyl)-5-methoxy-5-methylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoate (230 mg, 0.29 mmol, 1.00 equiv), methanol (3.00 mL), dioxane (3.00 mL), 4M NaOH(0.44 mL, 1.75 mmol, 6.Oequiv). The resulting solution was stirred for 3 h at 70° C. The reaction mixture was cooled to RT. The resulting solution was diluted with 10 mL of H₂O. The pH value of the solution was adjusted to 5 with HOAc. The resulting solution was extracted with 4×10 mL of ethyl acetate and the organic layers combined. The resulting mixture was washed with 20 mL of brine. The mixture was dried over anhydrous sodium sulfate. The residue was applied onto a silica gel column with ethyl acetate/petroleum ether (1:2-2:1). This resulted in 150 mg (66.40%) of 4-(4-[[2-(4-chlorophenyl)-5-methoxy-5-methylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid as brown solid. LC-MS: (ES, m/z): 772 [M+H]⁺.

Synthesis of 4-([[(2S)-1,4-dioxan-2-yl]methyl]amino)-3-nitrobenzene-1-sulfonamide: Into a 100-mL round-bottom flask, was placed 4-fluoro-3-nitrobenzene-1-sulfonamide (1.43 g, 0.007 mmol, 1 equiv), 1-[(2S)-1,4-dioxan-2-yl]methanamine hydrochloride (1 g, 6.510 mmol, 1 equiv), THE (30 mL), Cs₂CO₃ (8.48 g, 0.026 mmol, 4 equiv). The resulting solution was stirred overnight at 50° C. in an oil bath. The solids were collected by filtration. The solid was dried in an oven under reduced pressure. This resulted in 1.82 g (88.10%) of 4-([[(2S)-1,4-dioxan-2-yl]methyl]amino)-3-nitrobenzene-1-sulfonamide as yellow solid. LC-MS: (ES, m/z): M+1:318.

Synthesis of 4-(4-[[2-(4-chlorophenyl)-5-methoxy-5-methylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide: Into a 40-mL vial, was added 4-(4-[[2-(4-chlorophenyl)-5-methoxy-5-methylcyclohex-1-en-1-yl]methyl]piperazin-1-yl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzoic acid (140.00 mg, 0.18 mmol, 1.00 equiv), DCM (2.00 mL), DMAP (44.28 mg, 0.36 mmol, 2 equiv), 4-[[(2S)-1,4-dioxan-2-ylmethyl]amino]-3-nitrobenzenesulfonamide (69.00 mg, 0.21 mmol, 1.20 equiv), EDCI (41.8 mg, 0.21 mmol, 1.20 equiv). The resulting solution was stirred for 12 h at 30° C. The resulting solution was diluted with 10 mL of H₂O. The resulting solution was extracted with 4×10 mL of ethyl acetate and the organic layers combined. The resulting mixture was washed with 1×20 mL of brine. The mixture was dried over anhydrous sodium sulfate and concentrated. The residue was applied onto Pre-TLC with dichloromethane/methanol (95:5).

This resulted in 135 mg (69.50%) of 4-(4-[[2-(4-chlorophenyl)-5-methoxy-5-methylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide as yellow solid. LC-MS: (ES, m/z): 1071 [M+H]⁺.

Synthesis of 4-(4-[[2-(4-chlorophenyl)-5-methoxy-5-methylcyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide: Into a 40-mL vial, was added 4-(4-[[2-(4-chlorophenyl)-5-methoxy-5-methylcyclohex-1-en-1-yl]methyl]piperazin-1-yl)-N-(4-[[(2S)-1,4-dioxan-2-ylmethyl]amino]-3-nitrobenzenesulfonyl)-2-(4-[[2-(trimethylsilyl)ethoxy]methyl]-14-oxa-2,4,10-triazatricyclo[7.5.0.0\hat{ }[3,7]]tetradeca-1(9),2,5,7-tetraen-10-yl)benzamide (135 mg, 0.125 mmol, 1.00 equiv), THE (4.00 mL), ethylenediamine (150.00 mg, 2.50 mmol, 20.Oequiv), TBAF 1M in THF (2.50 mL, 2.50 mmol, 20.Oequiv). The resulting solution was stirred for 8 h at 700° C. The reaction mixture was cooled to RT. The resulting solution was diluted with 10 mL of H₂O. The resulting solution was extracted with 4×10 mL of ethyl acetate and the organic layers combined. The resulting mixture was washed with 20 mL of brine. The mixture was dried over anhydrous sodium sulfate and concentrated. The residue was applied onto a silica gel column with dichloromethane/methanol (95:5). The crude product (50 mg) was purified by Flash-Prep-HPLC with the following conditions (IntelFlash-1): Column, C18 silica gel; mobile phase, CH₃CN: H₂O (0.05NH₃.H₂O)=10% increasing to CH₃CN: H₂O(0.05NH₃.H₂O)=50 within 6; Detector, 220. 23 mg product was obtained. This resulted in 23 mg (25.30%) of 4-(4-[[2-(4-chlorophenyl)-5-methoxy-5-methylcyclohex-1-en-1-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide as yellow solid. LC-MS: (ES, m/z): 941 [M+H]⁺ ¹H NMR (300 MHz, DMSO, ppm): δ11.93 (s, 1H), 8.81 (s, 1H), 8.70 (s, 1H), 8.45 (s, 1H), 7.98 (m, 2H), 7.31 (s, 1H), 7.13 (s, 1H), 7.08-6.94 (m, 3H), 6.74 (s, 2H), 6.66 (m, 1H), 6.20 (s, 1H), 4.08-3.54 (m, 8H), 3.47 (m, 2H), 3.29 (s, 8H), 2.84 (s, 2H), 2.61-2.04 (m, 10H), 1.89 (m, 1H), 1.80-1.63 (m, 2H), 1.28 (s, 3H).

### Compound 4-1: Preparation of 4-(4-[[4-(4-chlorophenyl)-6,6-dimethyl-2,5-dihydropyran-3-yl]methyl]piperazin-1-yl)-N-(4-[[(2S)-1,4-dioxan-2-ylmethyl]amino]-3-nitrobenzenesulfonyl)-2-[14-oxa-2,4,10-triazatricyclo[7.5.0.0\hat{ }[3,7]]tetradeca-1(9),2,5,7-tetraen-10-yl]benzamide

Synthesis of (E)-2-(2-ethoxyvinyl)-4,4,5,5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil. LC-MS: (ES, m/z): M+1=199; H-NMR (300 MHz, DMSO-d₆, ppm) δ6.91-6.95 (m, 1H), 4.30-4.34 (m, 1H), 3.78-3.82 (m, 2H), 1.14-1.24 (m, 15H).

Synthesis of N-[(2R)-2-hydroxypropyl]acetamide: Into a 250-mL 3-necked round-bottom flask purged and maintained with an inert atmosphere of nitrogen, was placed (2R)-1-aminopropan-2-ol (100 g, 1.3 mol, 1 equiv), DCM (1 L), TEA (160 g, 1.6 mol, 1.2 equiv). This was followed by the addition of a solution of acetyl acetate (136 g, 1.3 mol, 1.0 equiv) in 100 ml (DCM) dropwise with stirring at 0-10° C. The resulting solution was stirred for 14 hr at room temperature. The resulting mixture was concentrated. The residue was applied onto a silica gel column with dichloromethane/methanol (100:5). This resulted in 250 g (crude) of N-[(2R)-2-hydroxypropyl]acetamide as a yellow oil.

Synthesis of 5-bromo-6-fluoropyridin-2-amine: Into a 20L 4-necked round-bottom flask, was placed 6-fluoropyridin-2-amine (4500.00 g, 40.178 mol, 1.00 equiv), ACN (20.00 L). This was followed by the addition of NBS (7035.17 g, 41.383 mol, 1.03 equiv) in portions at R.T (25) degrees C. The resulting solution was stirred for 2 h at room temperature. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as a light brown solid. LC-MS: (ES, m/z): M+1=190; H-NMR (300 MHz, DMSO-d₆, ppm) δ7.63-7.71 (m, 1H), 6.56 (s, 2H), 6.30-631 (m, 1H).

Synthesis of 5-bromo-6-fluoro-3-iodopyridin-2-amine: Into a 20L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 2h at room temperature. The resulting solution was diluted with 100 L of water. The mixture was filtrated, collection of filter cake and the filter cake was washed by water (35L×2) and dried by oven to give product 9840 g (Y=90%) of 5-bromo-6-fluoro-3-iodopyridin-2-amine as a brown solid. LC-MS: (ES, m/z): M+1=317; H-NMR (300 MHz, DMSO-d₆, ppm) δ8.16-8.23 (m, 1H), 6.69 (s, 2H).

Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature in a liquid nitrogen bath. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil. LC-MS: (ES, m/z) M+1=261; H-NMR (300 MHz, DMSO-d₆, ppm) δ7.96-7.99 (m, 1H), 6.72-6.76 (m, 1H), 5.49-5.53 (m, 1H), 3.99-4.06 (m, 3H).

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as a light brown solid. LC-MS: (ES, m/z): M+1=215; H-NMR (300 MHz, DMSO-d₆, ppm) δ9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS (ES, m/z) M+1=345; H-NMR (300 MHz, DMSO-d₆, ppm) δ8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

Synthesis of (R)-N-(2-(5-bromo-1-((2-(trimethylsilyl)ethoxy)methyl)-1H-pyrrolo[2,3-b]pyidin-6-yloxy)propyl)acetamide: Into a 50-mL round-bottom flask, was placed N-[(2R)-2-hydroxypropyl] acetamide (crude, 81.6 g, 697 mmol, 3.00 equiv), dioxane (800 ml). This was followed by the addition of NaH (28 g, 697 mmol, 3 equiv), in portions at 5° C. 15° C. The resulting solution was stirred for 30 min at RT. To this was added a solution of 5-bromo-6-fluoro-1-((2-(trimethylsilyl)ethoxy)methyl)-1H-pyrrolo[2,3-b] pyridine (80 g, 232 mmol, 1 equiv) in dioxane (100 ml). The resulting solution was stirred for 4 hr at 80° C. in an oil bath. The reaction was then quenched by the addition of 50 ml of water at 10° C. The resulting solution was concentrated and diluted with 500 ml H₂O. The resulting solution was extracted with 3×500 ml of ethyl acetate. The resulting mixture was washed with 1×300 ml of NaCl (aq). The mixture was dried over anhydrous sodium sulfate and concentrated. The residue was applied onto a silica gel column with PE/EA (100:20). This resulted in 40 g (pure) and 30 g (70%) of (R)-N-(2-(5-bromo-1-((2-(trimethylsilyl)ethoxy)methyl)-1H-pyrrolo[2,3-b]pyridin-6-yloxy)propyl)acetamide as a yellow oil.

### Synthesis of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one: Into a 1-mL round-bottom flask purged and maintained with an inert atmosphere of nitrogen, was placed (R)-N-(2-(5-bromo-1-((2-(trimethylsilyl)ethoxy)methyl)-1H-pyrrolo[2,3-b]pyridin-6-yloxy)propyl)acetamide (40 g, 90.7 mmol, 1 equiv), dioxane (500 mL), Cs₂CO₃ (88 g, 272 mmol, 3 equiv), BrettPhos Pd G3 (4.1 g, 4.5 mmol, 0.05 equiv). The resulting solution was concentrated and diluted with 300 ml H₂O. The resulting solution was extracted with 3×300 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (40:100). This resulted in 33 g(crude, 70%) of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one as brown oil

Synthesis of (12R)-4-(trimethylsilyl)ethoxy)methyl)-12-methyl-13-oxa-2,4,10-triazatricyclo[7.4.0.0\hat{ }[3,7]]trideca-1(9),2,5,7-tetraene: Into a 1-L vial, was placed 1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one (33 g), methanol (300 mL), sodium hydroxide (2M, 300 ml). The resulting solution was stirred for 6 hr at 80° C. in an oil bath. v). The resulting solution was concentrated and remove MeOH, then extracted with 3×200 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (1:1). This resulted in 16.2 g of (12R)-4-(trimethylsilyl)ethoxy)methyl)-12-methyl-13-oxa-2,4,10-triazatricyclo [7.4.0.0\hat{ }[3,7]]trideca-1(9),2,5,7-tetraene as brown oil.

Synthesis of 2-bromo-4-[4-(tert-butoxycarbonyl)piperazin-1-yl]benzoic acid: Into a 100-mL 3-necked round-bottom flask, was placed tert-butyl 4-[3-bromo-4-(methoxycarbonyl)phenyl]piperazine-1-carboxylate (4.00 g, 10.0 mmol, 1.00 equiv,), MeOH(20 ml), THF(20 ml), H₂O (20 ml), NaOH (1.60 g, 40.0 mmol, 4.00 equiv,). The resulting solution was stirred for 3 h at 30 degrees C. The resulting solution was diluted with 40 mL of H₂O. The pH value of the solution was adjusted to 5 with HCl (0.5 mol/L). The resulting solution was extracted with 3×30 mL of ethyl acetateand the organic layers combined. The resulting mixture was washed with 1×50 mL of brine. The mixture was dried over anhydrous sodium sulfate and concentrated. This resulted in 3.8 g (98.45%) of 2-bromo-4-[4-(tert-butoxycarbonyl)piperazin-1-yl]benzoic acid as a white solid. LC-MS: (ES, m/z): M+1: 385.

Synthesis of tert-butyl 4-[3-bromo-4-([3-nitroso-4-[(oxan-4-ylmethyl)amino]benzenesulfonyl]carbamoyl)phenyl]piperazine-1-carboxylate hydrate: Into a 100-mL 3-necked round-bottom flask, was placed 2-bromo-4-[4-(tert-butoxycarbonyl)piperazin-1-yl]benzoic acid (3.80 g, 9.864 mmol, 1.00 equiv), 3-nitro-4-[(oxan-4-ylmethyl)amino]benzenesulfonamide (3.11 g, 9.864 mmol, 1 equiv), EDCI (2.27 g, 11.836 mmol, 1.2 equiv), DMAP (2.41 g, 19.727 mmol, 2 equiv), DCM (40.00 mL). The resulting solution was stirred for 12 h at 30 degrees C. The resulting solution was diluted with 50 mL of H₂O. The resulting solution was extracted with 3×50 mL of dichloromethane and the organic layers combined. The resulting mixture was washed with 1×50 mL of brine. The mixture was dried over anhydrous sodium sulfate and concentrated. The residue was applied onto a silica gel column with dichloromethane/methanol (90:10). This resulted in 5.4 g (79.73%) of tert-butyl 4-[3-bromo-4-([3-nitroso-4-[(oxan-4-ylmethyl)amino]benzenesulfonyl]carbamoyl)phenyl]piperazine-1-carboxylate hydrate as yellow solid.LC-MS: (ES, m/z): M+1:682.

Synthesis of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitroso-4-[(oxan-4-ylmethyl)amino]benzenesulfonyl]carbamoyl)phenyl]piperazine-1-carboxylate hydrate: Into a 100-mL round-bottom flask purged and maintained with an inert atmosphere of nitrogen, was placed tert-butyl 4-[3-bromo-4-([3-nitro-4-[(oxan-4-ylmethyl)amino]benzenesulfonyl]carbamoyl)phenyl]piperazine-1-carboxylate (4.00 g, 5.860 mmol, 1.00 equiv), (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (1.87 g, 5.860 mmol, 1.00 equiv), DMF (60.00 mL), CuI (0.22 g, 1.172 mmol, 0.20 equiv), Cs₂CO₃ (3.82 g, 11.720 mmol, 2.00 equiv), N1,N2-bis(4-hydroxy-2,6-dimethylphenyl)oxalamide (0.58 g, 1.766 mmol, 0.30 equiv). The resulting solution was stirred for 2 h at 100 degrees C. The reaction mixture was cooled to room temperature with a water bath. The resulting solution was diluted with 100 mL of H₂O. The resulting solution was extracted with 3×100 mL of ethyl acetate and the organic layers combined and dried over anhydrous sodium sulfate and concentrated. The residue was applied onto a silica gel column with ethyl acetate/petroleum ether (1:3-1:0). This resulted in 3.2 g (59.02%) of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitroso-4-[(oxan-4-ylmethyl)amino]benzenesulfonyl]carbamoyl)phenyl]piperazine-1-carboxylate hydrate as yellow solid. LC-MS: (ES, m/z): M+1:921.

Synthesis of 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide: Into a 100-mL round-bottom flask, was placed tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitro-4-[(oxan-4-ylmethyl)amino]benzenesulfonyl]carbamoyl)phenyl]piperazine-1-carboxylate (2.40 g, 2.605 mmol, 1.00 equiv), DCM (30.00 mL), TFA (10.00 mL),The resulting solution was stirred for 4 h at RT. The resulting mixture was concentrated. Added CH₃CN (30.00 mL), ethylenediamine (0.79 g, 13.145 mmol, 5.05 equiv). The resulting solution was allowed to react for an additional 6 h at 60 degrees C. The resulting mixture was concentrated. The crude product (3.0 g) was purified by Flash-Prep-HPLC with the following conditions (CombiFlash-1): Column, C18 silica gel; mobile phase, CH₃CN: H₂O (0.5% FA)=5% increasing to CH₃CN:H₂O(0.5% FA)=60 within 7 min; Detector, UV 254 nm. This resulted in 600 mg (33.34%) of 2-[(12R)-12-methyl-3-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide as yellow solid. LC-MS: (ES, m/z): M+1:691.

Synthesis of 2-bromo-1,4,4-trimethylcyclohex-1-ene hydrate: Into a 250-mL 3-necked round-bottom flask, was placed CHCl₃(100.00 mL), DMF (7.24 g, 99.050 mmol, 2.5 equiv). This was followed by the addition of PBr₃ (24.65 g, 91.065 mmol, 2.30 equiv) dropwise with stirring. The resulting solution was stirred for 1 hr at room temperature. Then to this was added cyclohexanone, 3,3-dimethyl-(5.00 g, 39.620 mmol, 1.00 equiv) dropwise with stirring at 0 degrees C. The resulting solution was allowed to react, for 12 hr at RT. The reaction was then pulled into 200 mL of water/ice. The pH value of the solution was adjusted to 5 with Sat.Na₂CO₃. The resulting solution was extracted with 3×100 mL of dichloromethane and the organic layers combined and dried over anhydrous sodium sulfate and concentrated. This resulted in 3.1 g (35.38%) of 2-bromo-1,4,4-trimethylcyclohex-1-ene hydrate as colorless oil.LC-MS: (ES, m/z): M+1:217/219.

Synthesis of 2-(4-chloro-3-fluorophenyl)-4,4-dimethylcyclohex-1-ene-1-carbaldehyde: Into a 40-mL vial purged and maintained with an inert atmosphere of nitrogen, was placed 2-bromo-4,4-dimethylcyclohex-1-ene-1-carbaldehyde (300.00 mg, 1.382 mmol, 1.00 equiv), 4-chloro-3-fluorophenylboronic acid (289.12 mg, 1.658 mmol, 1.2 equiv), DME (4.00 mL), Na₂CO₃ (292.91 mg, 2.764 mmol, 2 equiv), Pd(PPh₃)₂Cl₂ (48.49 mg, 0.069 mmol, 0.05 equiv), H₂O (0.20 mL). The resulting solution was stirred for 12 h at 100 degrees C. The reaction mixture was cooled to room temperature. The resulting mixture was concentrated. The residue was applied onto a silica gel column with ethyl acetate/petroleum ether (0:100-1:6). This resulted in 170 mg (46.12%) of 2-(4-chloro-3-fluorophenyl)-4,4-dimethylcyclohex-1-ene-1-carbaldehyde as colorless oil.LC-MS: (ES, m/z): M+1:267.

Synthesis of 4-(4-[[2-(4-chloro-3-fluorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial, was placed 2-(4-chloro-3-fluorophenyl)-4,4-dimethylcyclohex-1-ene-1-carbaldehyde (55.00 mg, 0.21 mmol, 1.00 equiv), [(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide (142.43 mg, 0.206 mmol, 1 equiv), MeOH (1.50 mL), DCM (1.50 mL), ZnCl₂ (56.21 mg, 0.412 mmol, 2 equiv), NaBH₃CN (25.92 mg, 0.412 mmol, 2 equiv). The resulting solution was stirred for 5 h at 60 degrees C. The reaction mixture was cooled to room temperature. The resulting mixture was concentrated. The residue was applied onto Pre-TLC with dichloromethane/methanol (95:5). The crude product (50 mg) was purified by Flash-Prep-HPLC with the following conditions (IntelFlash-1): Column, C18 silica gel; mobile phase, CH₃CN: H₂O(0.05% NH₃.H₂O)=10% increasing to CH₃CN:H₂O(0.05% NH₃.H₂O)=60% within 7 min; Detector, UV 254 nm. This resulted in 6.0 mg (3.09%) of 4-(4-[[2-(4-chloro-3-fluorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as yellow solid.LC-MS: (ES, m/z): M+1:941. ¹H NMR (300 MHz, DMSO-d₆, ppm) δ10.89 (s, 1H), 8.48-8.29 (m, 2H), 7.51 (dd, J=8.6, 7.1 Hz, 2H), 7.33 (s, 1H), 7.11 (dd, J=10.3, 1.8 Hz, 1H), 7.00 (t, J=2.8 Hz, 1H), 6.92 (dd, J=8.2, 1.9 Hz, 1H), 6.69 (d, J=24.5 Hz, 4H), 5.99 (s, 1H), 4.41 (s, 1H), 3.86 (dd, J=11.5, 4.1 Hz, 2H), 3.29-3.17 (m, 3H), 3.10 (d, J=11.7 Hz, 6H), 2.77 (s, 2H), 2.21 (d, J=19.8 Hz, 6H), 1.99 (s, 2H), 1.89 (d, J=12.7 Hz, 1H), 1.63 (d, J=13.0 Hz, 2H), 1.40 (t, J=6.2 Hz, 2H), 1.35-1.10 (m, 6H), 0.94 (s, 6H).

### Compound 4-2: Preparation of 4-(4-[[2-(4-chloro-2-fluorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide

Synthesis of (E)-2-(2-ethoxyvinyl)-4,4,5,5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil. LC-MS: (ES, m/z): M+1=199; H-NMR (300 MHz, DMSO-d₆, ppm) S 6.91-6.95 (m, 1H), 4.30-4.34 (m, 1H), 3.78-3.82 (m, 2H), 1.14-1.24 (m, 15H).

Synthesis of N-[(2R)-2-hydroxypropyl]acetamide: Into a 250-mL 3-necked round-bottom flask purged and maintained with an inert atmosphere of nitrogen, was placed (2R)-1-aminopropan-2-ol (100 g, 1.3 mol, 1 equiv), DCM (1 L), TEA (160 g, 1.6 mol, 1.2 equiv). This was followed by the addition of a solution of acetyl acetate (136 g, 1.3 mol, 1.0 equiv) in 100 ml (DCM) dropwise with stirring at 0-10° C. The resulting solution was stirred for 14 hr at room temperature. The resulting mixture was concentrated. The residue was applied onto a silica gel column with dichloromethane/methanol (100:5). This resulted in 250 g (crude) of N-[(2R)-2-hydroxypropyl]acetamide as a yellow oil.

Synthesis of 5-bromo-6-fluoropyridin-2-amine: Into a 20L 4-necked round-bottom flask, was placed 6-fluoropyridin-2-amine (4500.00 g, 40.178 mol, 1.00 equiv), ACN (20.00 L). This was followed by the addition of NBS (7035.17 g, 41.383 mol, 1.03 equiv) in portions at R.T (25) degrees C. The resulting solution was stirred for 2 h at room temperature. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as a light brown solid. LC-MS: (ES, m/z): M+1=190; H-NMR (300 MHz, DMSO-d₆, ppm) δ7.63-7.71 (m, 1H), 6.56 (s, 2H), 6.30-631 (m, 1H).

Synthesis of 5-bromo-6-fluoro-3-iodopyridin-2-amine: Into a 20L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 2h at room temperature. The resulting solution was diluted with 100 L of water. The mixture was filtrated, collection of filter cake and the filter cake was washed by water (35L×2) and dried by oven to give product 9840 g (Y=90%) of 5-bromo-6-fluoro-3-iodopyridin-2-amine as a brown solid. LC-MS: (ES, m/z): M+1=317; H-NMR (300 MHz, DMSO-d₆, ppm) δ8.16-8.23 (m, 1H), 6.69 (s, 2H).

Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature in a liquid nitrogen bath. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil. LC-MS: (ES, m/z) M+1=261; H-NMR (300 MHz, DMSO-d₆, ppm) δ7.96-7.99 (m, 1H), 6.72-6.76 (m, 1H), 5.49-5.53 (m, 1H), 3.99-4.06 (m, 3H).

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as a light brown solid. LC-MS: (ES, m/z): M+1=215; H-NMR (300 MHz, DMSO-d₆, ppm) δ9.53 (brs, 1H), 8.19-8.22 (d, J=9.0 Hz, 1H), 7.32-7.34 (m, 1H), 6.50-6.52 (m, 1H).

### Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS (ES, m/z) M+1=345; H-NMR (300 MHz, DMSO-d₆, ppm) δ8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H)

Synthesis of (R)-N-(2-(5-bromo-1-((2-(trimethylsilyl)ethoxy)methyl)-1H-pyrr olo[2,3-b]pyidin-6-yloxy)propyl)acetamide: Into a 50-mL round-bottom flask, was placed N-[(2R)-2-hydroxypropyl] acetamide (crude, 81.6 g, 697 mmol, 3.00 equiv), dioxane (800 ml). This was followed by the addition of NaH (28 g, 697 mmol, 3 equiv), in portions at 5° C. 15° C. The resulting solution was stirred for 30 min at RT. To this was added a solution of 5-bromo-6-fluoro-1-((2-(trimethylsilyl)ethoxy)methyl)-1H-pyrrolo[2,3-b] pyridine (80 g, 232 mmol, 1 equiv) in dioxane (100 ml). The resulting solution was stirred for 4 hr at 80° C. in an oil bath. The reaction was then quenched by the addition of 50 ml of water at 10° C. The resulting solution was concentrated and diluted with 500 ml H₂O. The resulting solution was extracted with 3×500 ml of ethyl acetate. The resulting mixture was washed with 1×300 ml of NaCl (aq). The mixture was dried over anhydrous sodium sulfate and concentrated. The residue was applied onto a silica gel column with PE/EA (100:20). This resulted in 40 g (pure) and 30 g (70%) of (R)-N-(2-(5-bromo-1-((2-(trimethylsilyl)ethoxy)methyl)-1H-pyrrolo[2,3-b]pyridin-6-yloxy)propyl)acetamide as a yellow oil.

Synthesis of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one: Into a 1-mL round-bottom flask purged and maintained with an inert atmosphere of nitrogen, was placed (R)-N-(2-(5-bromo-1-((2-(trimethylsilyl)ethoxy)methyl)-1H-pyrrolo[2,3-b]pyridin-6-yloxy)propyl)acetamide (40 g, 90.7 mmol, 1 equiv), dioxane (500 mL), Cs₂CO₃ (88 g, 272 mmol, 3 equiv), BrettPhos Pd G3 (4.1 g, 4.5 mmol, 0.05 equiv). The resulting solution was concentrated and diluted with 300 ml H₂O. The resulting solution was extracted with 3×300 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (40:100). This resulted in 33 g(crude, 70%) of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one as brown oil.

Synthesis of (12R)-4-(trimethylsilyl)ethoxy)methyl)-12-methyl-13-oxa-2,4,10-triazatricyclo[7.4.0.0\hat{ }[3,7]]trideca-1(9),2,5,7-tetraene: Into a 1-L vial, was placed1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one (33 g), methanol (300 mL), sodium hydroxide (2M, 300 ml). The resulting solution was stirred for 6 hr at 80° C. in an oil bath. v). The resulting solution was concentrated(MeOH) and extracted with 3×200 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (1:1). This resulted in 16.2 g of (12R)-4-(trimethylsilyl)ethoxy)methyl)-12-methyl-13-oxa-2,4,10-triazatricyclo [7.4.0.0\hat{ }[3,7]]trideca-1(9),2,5,7-tetraene as brown oil.

Synthesis of 2-bromo-4-[4-(tert-butoxycarbonyl)piperazin-1-yl]benzoic acid: Into a 100-mL 3-necked round-bottom flask, was placed tert-butyl 4-[3-bromo-4-(methoxycarbonyl)phenyl]piperazine-1-carboxylate (4.00 g, 10.0 mmol, 1.00 equiv,), MeOH(20 ml), THF(20 ml), H₂O (20 ml), NaOH (1.60 g, 40.0 mmol, 4.00 equiv,). The resulting solution was stirred for 3 h at 30 degrees C. The resulting solution was diluted with 40 mL of H₂O. The pH value of the solution was adjusted to 5 with HCl (0.5 mol/L). The resulting solution was extracted with 3×30 mL of ethyl acetateand the organic layers combined. The resulting mixture was washed with 1×50 mL of brine. The mixture was dried over anhydrous sodium sulfate and concentrated. This resulted in 3.8 g (98.45%) of 2-bromo-4-[4-(tert-butoxycarbonyl)piperazin-1-yl]benzoic acid as a white solid. LC-MS: (ES, m/z): M+1: 385.

Synthesis of tert-butyl 4-[3-bromo-4-([3-nitro-4-[(oxan-4-ylmethyl)amino]benzenesulfonyl]carbamoyl)phenyl]piperazine-1-carboxylate hydrate: Into a 100-mL 3-necked round-bottom flask, was placed 2-bromo-4-[4-(tert-butoxycarbonyl)piperazin-1-yl]benzoic acid (3.80 g, 9.864 mmol, 1.00 equiv), 3-nitro-4-[(oxan-4-ylmethyl)amino]benzenesulfonamide (3.11 g, 9.864 mmol, 1 equiv), EDCI (2.27 g, 11.836 mmol, 1.2 equiv), DMAP (2.41 g, 19.727 mmol, 2 equiv), DCM (40.00 mL). The resulting solution was stirred for 12 h at 30 degrees C. The resulting solution was diluted with 50 mL of H₂O. The resulting solution was extracted with 3×50 mL of dichloromethane and the organic layers combined. The resulting mixture was washed with 1×50 mL of brine. The mixture was dried over anhydrous sodium sulfate and concentrated. The residue was applied onto a silica gel column with dichloromethane/methanol (90:10). This resulted in 5.4 g (79.73%) of tert-butyl 4-[3-bromo-4-([3-nitro-4-[(oxan-4-ylmethyl)amino]benzenesulfonyl]carbamoyl)phenyl]piperazine-1-carboxylate hydrate as yellow solid.LC-MS: (ES, m/z): M+1:682.

Synthesis of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitro-4-[(oxan-4-ylmethyl)amino]benzenesulfonyl]carbamoyl)phenyl]piperazine-1-carboxylate hydrate:Into a 100-mL round-bottom flask purged and maintained with an inert atmosphere of nitrogen, was placed tert-butyl 4-[3-bromo-4-([3-nitro-4-[(oxan-4-ylmethyl)amino]benzenesulfonyl]carbamoyl)phenyl]piperazine-1-carboxylate (4.00 g, 5.860 mmol, 1.00 equiv), (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (1.87 g, 5.860 mmol, 1.00 equiv), DMF (60.00 mL), CuI (0.22 g, 1.172 mmol, 0.20 equiv), Cs₂CO₃ (3.82 g, 11.720 mmol, 2.00 equiv), N1,N2-bis(4-hydroxy-2,6-dimethylphenyl)oxalamide (0.58 g, 1.766 mmol, 0.30 equiv). The resulting solution was stirred for 2 hr at 100 degrees C. The reaction mixture was cooled to room temperature with a water bath. The resulting solution was diluted with 100 mL of H₂O. The resulting solution was extracted with 3×100 mL of ethyl acetate and the organic layers combined and dried over anhydrous sodium sulfate and concentrated. The residue was applied onto a silica gel column with ethyl acetate/petroleum ether (1:3-1:0). This resulted in 3.2 g (59.02%) of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitroso-4-[(oxan-4-ylmethyl)amino]benzenesulfonyl]carbamoyl)phenyl]piperazine-1-carboxylate hydrate as yellow solid.LC-MS: (ES, m/z): M+1:921.

### Synthesis of 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide: Into a 100-mL round-bottom flask, was placed tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitro-4-[(oxan-4-ylmethyl)amino]benzenesulfonyl]carbamoyl)phenyl]piperazine-1-carboxylate (2.40 g, 2.605 mmol, 1.00 equiv), DCM (30.00 mL), TFA (10.00 mL),The resulting solution was stirred for 4 h at RT. The resulting mixture was concentrated. Added CH₃CN (30.00 mL), ethylenediamine (0.79 g, 13.145 mmol, 5.05 equiv). The resulting solution was allowed to react for an additional 6 h at 60 degrees C. The resulting mixture was concentrated. The crude product (3.0 g) was purified by Flash-Prep-HPLC with the following conditions (CombiFlash-1): Column, C18 silica gel; mobile phase, CH₃CN: H₂O(0.5% FA)=5% increasing to CH₃CN:H₂O(0.5% FA)=60 within 7 min; Detector, UV 254 nm. This resulted in 600 mg (33.34%) of 2-[(12R)-12-methyl-3-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide as yellow solid.LC-MS: (ES, m/z): M+1:691

Synthesis of 2-bromo-1,4,4-trimethylcyclohex-1-ene hydrate: Into a 250-mL 3-necked round-bottom flask, was placed CHCl₃(100.00 mL), DMF (7.24 g, 99.050 mmol, 2.5 equiv). This was followed by the addition of PBr₃ (24.65 g, 91.065 mmol, 2.30 equiv) dropwise with stirring. The resulting solution was stirred for 1 hr at room temperature. Then to this was added cyclohexanone, 3,3-dimethyl-(5.00 g, 39.620 mmol, 1.00 equiv) dropwise with stirring at 0 degrees C. The resulting solution was allowed to react, for 12 hr at RT. The reaction was then pulled into 200 mL of water/ice. The pH value of the solution was adjusted to 5 with Sat.Na₂CO₃. The resulting solution was extracted with 3×100 mL of dichloromethane and the organic layers combined and dried over anhydrous sodium sulfate and concentrated. This resulted in 3.1 g (35.38%) of 2-bromo-1,4,4-trimethylcyclohex-1-ene hydrate as colorless oil. LC-MS: (ES, m/z): M+1:217/219.

### Synthesis of 2-(4-chloro-2-fluorophenyl)-4,4-dimethylcyclohex-1-ene-1-carbaldehyde: Into a 40-mL vial purged and maintained with an inert atmosphere of nitrogen, was placed 2-bromo-4,4-dimethylcyclohex-1-ene-1-carbaldehyde (200.00 mg, 0.921 mmol, 1.00 equiv), 4-chloro-2-fluorophenylboronic acid (192.75 mg, 1.105 mmol, 1.2 equiv), Na₂CO₃ (195.27 mg, 1.842 mmol, 2.00 equiv), Pd(PPh₃)₂Cl₂ (32.33 mg, 0.046 mmol, 0.05 equiv), DME (4.00 mL), H₂O (0.20 mL). The resulting solution was stirred for 12 h at 100 degrees C. The reaction mixture was cooled to room temperature. The resulting mixture was concentrated. The residue was applied onto a silica gel column with ethyl acetate/petroleum ether (0:100-1:6). This resulted in 130 mg (52.91%) of 2-(4-chloro-2-fluorophenyl)-4,4-dimethylcyclohex-1-ene-1-carbaldehyde as colorless oil. LC-MS: (ES, m/z): M+1:267

Synthesis of 4-(4-[[2-(4-chloro-2-fluorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial, was placed 2-(4-chloro-2-fluorophenyl)-4,4-dimethylcyclohex-1-ene-1-carbaldehyde (55.00 mg, 0.21 mmol, 1.00 equiv), 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide (142.43 mg, 0.206 mmol, 1 equiv), MeOH (1.50 mL), DCM (1.50 mL), ZnCl₂ (56.21 mg, 0.412 mmol, 2 equiv), NaBH₃CN (25.92 mg, 0.412 mmol, 2 equiv). The resulting solution was stirred for 5 h at 60 degrees C. The reaction mixture was cooled to room temperature. The resulting mixture was concentrated. The residue was applied Pre-TLC with dichloromethane/methanol (95:5). The crude product (38 mg) was purified by Flash-Prep-HPLC with the following conditions (IntelFlash-1): Column, C18 silica gel; mobile phase, CH₃CN:H₂O90.5% NH₃.H₂O)=10% increasing to CH₃CN:H₂O90.5% NH3.H₂O)=60% within 7 min; Detector, UV 254 nm. This resulted in 3.1 mg (1.60%) of 4-(4-[[2-(4-chloro-2-fluorophenyl)-4,4-dimethylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as yellow solid. LC-MS: (ES, m/z): M+1:941. ¹H NMR (300 MHz, DMSO-d₆, ppm) δ12.49 (s, 1H), 8.70-8.56 (m, 2H), 8.45 (s, 1H), 8.11 (d, J=10.2 Hz, 2H), 7.78 (d, J=9.2 Hz, 1H), 7.14-6.99 (m, 2H), 6.98-6.81 (m, 2H), 6.75 (s, 1H), 6.67 (d, J=9.3 Hz, 1H), 6.55 (s, 1H), 6.12 (s, 1H), 3.44 (t, J=12.6 Hz, 4H), 3.22 (d, J=5.9 Hz, 4H), 2.75 (s, 2H), 2.49-2.14 (m, 4H), 2.12-1.86 (m, 3H), 1.68 (dd, J=37.2, 10.2 Hz, 4H), 1.57-1.40 (m, 4H), 0.98 (s, 6H), 0.89 (q, J=8.1, 7.0 Hz, 8H).

### Compound 4-3: Preparation of 4-[4-([4,4-dimethyl-2-[4-(trifluoromethyl)phenyl]cyclohex-1-en-1-yl]methyl)piperazin-1-yl]-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide

Synthesis of (E)-2-(2-ethoxyvinyl)-4,4,5,5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil. LC-MS: (ES, m/z): M+1=199; H-NMR (300 MHz, DMSO-d₆, ppm) δ6.91-6.95 (m, 1H), 4.30-4.34 (m, 1H), 3.78-3.82 (m, 2H), 1.14-1.24 (m, 15H).

Synthesis of N-[(2R)-2-hydroxypropyl]acetamide: Into a 250-mL 3-necked round-bottom flask purged and maintained with an inert atmosphere of nitrogen, was placed (2R)-1-aminopropan-2-ol (100 g, 1.3 mol, 1 equiv), DCM (1 L), TEA (160 g, 1.6 mol, 1.2 equiv). This was followed by the addition of a solution of acetyl acetate (136 g, 1.3 mol, 1.0 equiv) in 100 ml (DCM) dropwise with stirring at 0-10° C. The resulting solution was stirred for 14 hr at room temperature. The resulting mixture was concentrated. The residue was applied onto a silica gel column with dichloromethane/methanol (100:5). This resulted in 250 g (crude) of N-[(2R)-2-hydroxypropyl]acetamide as a yellow oil.

Synthesis of 5-bromo-6-fluoropyridin-2-amine: Into a 20L 4-necked round-bottom flask, was placed 6-fluoropyridin-2-amine (4500.00 g, 40.178 mol, 1.00 equiv), ACN (20.00 L). This was followed by the addition of NBS (7035.17 g, 41.383 mol, 1.03 equiv) in portions at R.T (25) degrees C. The resulting solution was stirred for 2 h at room temperature. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as a light brown solid. LC-MS: (ES, m/z): M+1=190; H-NMR (300 MHz, DMSO-d₆, ppm) δ7.63-7.71 (m, 1H), 6.56 (s, 2H), 6.30-631 (m, 1H).

Synthesis of 5-bromo-6-fluoro-3-iodopyridin-2-amine: Into a 20L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 2h at room temperature. The resulting solution was diluted with 100 L of water. The mixture was filtrated, collection of filter cake and the filter cake was washed by water (35L×2) and dried by oven to give product 9840 g (Y=90%) of 5-bromo-6-fluoro-3-iodopyridin-2-amine as a brown solid. LC-MS: (ES, m/z): M+1=317; H-NMR (300 MHz, DMSO-d₆, ppm) δ8.16-8.23 (m, 1H), 6.69 (s, 2H).

### Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature in a liquid nitrogen bath. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil. LC-MS: (ES, m/z) M+1=261; H-NMR (300 MHz, DMSO-d₆, ppm) δ7.96-7.99 (m, 1H), 6.72-6.76 (m, 1H), 5.49-5.53 (m, 1H), 3.99-4.06 (m, 3H)

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as a light brown solid. LC-MS: (ES, m/z): M+1=215; H-NMR (300 MHz, DMSO-d₆, ppm) δ9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS (ES, m/z) M+1=345; H-NMR (300 MHz, DMSO-d₆, ppm) δ8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

Synthesis of (R)-N-(2-(5-bromo-1-((2-(trimethylsilyl)ethoxy)methyl)-1H-pyrrolo[2,3-b]pyidin-6-yloxy)propyl)acetamide: Into a 50-mL round-bottom flask, was placed N-[(2R)-2-hydroxypropyl] acetamide (crude, 81.6 g, 697 mmol, 3.00 equiv), dioxane (800 ml). This was followed by the addition of NaH (28 g, 697 mmol, 3 equiv), in portions at 5° C. 15° C. The resulting solution was stirred for 30 min at RT. To this was added a solution of 5-bromo-6-fluoro-1-((2-(trimethylsilyl)ethoxy)methyl)-1H-pyrrolo[2,3-b] pyridine (80 g, 232 mmol, 1 equiv) in dioxane (100 ml). The resulting solution was stirred for 4 hr at 80° C. in an oil bath. The reaction was then quenched by the addition of 50 ml of water at 10° C. The resulting solution was concentrated and diluted with 500 ml H₂O. The resulting solution was extracted with 3×500 ml of ethyl acetate. The resulting mixture was washed with 1×300 ml of NaCl (aq). The mixture was dried over anhydrous sodium sulfate and concentrated. The residue was applied onto a silica gel column with PE/EA (100:20). This resulted in 40 g (pure) and 30 g (70%) of (R)-N-(2-(5-bromo-1-((2-(trimethylsilyl)ethoxy)methyl)-1H-pyrrolo[2,3-b]pyridin-6-yloxy)propyl)acetamide as a yellow oil.

Synthesis of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one: Into a 1-mL round-bottom flask purged and maintained with an inert atmosphere of nitrogen, was placed (R)-N-(2-(5-bromo-1-((2-(trimethylsilyl)ethoxy)methyl)-1H-pyrrolo[2,3-b]pyridin-6-yloxy)propyl)acetamide (40 g, 90.7 mmol, 1 equiv), dioxane (500 mL), Cs₂CO₃ (88 g, 272 mmol, 3 equiv), BrettPhos Pd G3 (4.1 g, 4.5 mmol, 0.05 equiv). The resulting solution was concentrated and diluted with 300 ml H₂O. The resulting solution was extracted with 3×300 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (40:100). This resulted in 33 g(crude, 70%) of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one as brown oil.

Synthesis of (12R)-4-(trimethylsilyl)ethoxy)methyl)-12-methyl-13-oxa-2,4,10-triazatricyclo[7.4.0.0\hat{ }[3,7]]trideca-1(9),2,5,7-tetraene: Into a 1-L vial, was placed1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one (33 g), methanol (300 mL), sodium hydroxide (2M, 300 ml). The resulting solution was stirred for 6 hr at 80° C. in an oil bath. v). The resulting solution was concentrated(MeOH) and extracted with 3×200 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (1:1). This resulted in 16.2 g of (12R)-4-(trimethylsilyl)ethoxy)methyl)-12-methyl-13-oxa-2,4,10-triazatricyclo [7.4.0.0\hat{ }[3,7]]trideca-1(9),2,5,7-tetraene as brown oil.

Synthesis of 2-bromo-4-[4-(tert-butoxycarbonyl)piperazin-1-yl]benzoic acid: Into a 100-mL 3-necked round-bottom flask, was placed tert-butyl 4-[3-bromo-4-(methoxycarbonyl)phenyl]piperazine-1-carboxylate (4.00 g, 10.0 mmol, 1.00 equiv,), MeOH(20 ml), THF(20 ml), H₂O (20 ml), NaOH (1.60 g, 40.0 mmol, 4.00 equiv,). The resulting solution was stirred for 3 h at 30 degrees C. The resulting solution was diluted with 40 mL of H₂O. The pH value of the solution was adjusted to 5 with HCl (0.5 mol/L). The resulting solution was extracted with 3×30 mL of ethyl acetateand the organic layers combined. The resulting mixture was washed with 1×50 mL of brine. The mixture was dried over anhydrous sodium sulfate and concentrated. This resulted in 3.8 g (98.45%) of 2-bromo-4-[4-(tert-butoxycarbonyl)piperazin-1-yl]benzoic acid as a white solid. LC-MS: (ES, m/z): M+1: 385.

Synthesis of tert-butyl 4-[3-bromo-4-([3-nitroso-4-[(oxan-4-ylmethyl)amino]benzenesulfonyl]carbamoyl)phenyl]piperazine-1-carboxylate hydrate: Into a 100-mL 3-necked round-bottom flask, was placed 2-bromo-4-[4-(tert-butoxycarbonyl)piperazin-1-yl]benzoic acid (3.80 g, 9.864 mmol, 1.00 equiv), 3-nitro-4-[(oxan-4-ylmethyl)amino]benzenesulfonamide (3.11 g, 9.864 mmol, 1 equiv), EDCI (2.27 g, 11.836 mmol, 1.2 equiv), DMAP (2.41 g, 19.727 mmol, 2 equiv), DCM (40.00 mL). The resulting solution was stirred for 12 h at 30 degrees C. The resulting solution was diluted with 50 mL of H₂O. The resulting solution was extracted with 3×50 mL of dichloromethane and the organic layers combined. The resulting mixture was washed with 1×50 mL of brine. The mixture was dried over anhydrous sodium sulfate and concentrated. The residue was applied onto a silica gel column with dichloromethane/methanol (90:10). This resulted in 5.4 g (79.73%) of tert-butyl 4-[3-bromo-4-([3-nitroso-4-[(oxan-4-ylmethyl)amino]benzenesulfonyl]carbamoyl)phenyl]piperazine-1-carboxylate hydrate as yellow solid.LC-MS: (ES, m/z): M+1:682.

Synthesis of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitroso-4-[(oxan-4-ylmethyl)amino]benzenesulfonyl]carbamoyl)phenyl]piperazine-1-carboxylate hydrate: Into a 100-mL round-bottom flask purged and maintained with an inert atmosphere of nitrogen, was placed tert-butyl 4-[3-bromo-4-([3-nitro-4-[(oxan-4-ylmethyl)amino]benzenesulfonyl]carbamoyl)phenyl]piperazine-1-carboxylate (4.00 g, 5.860 mmol, 1.00 equiv), (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (1.87 g, 5.860 mmol, 1.00 equiv), DMF (60.00 mL), CuI (0.22 g, 1.172 mmol, 0.20 equiv), Cs₂CO₃ (3.82 g, 11.720 mmol, 2.00 equiv), N1,N2-bis(4-hydroxy-2,6-dimethylphenyl)oxalamide (0.58 g, 1.766 mmol, 0.30 equiv). The resulting solution was stirred for 2 h at 100 degrees C. The reaction mixture was cooled to room temperature with a water bath. The resulting solution was diluted with 100 mL of H₂O. The resulting solution was extracted with 3×100 mL of ethyl acetate and the organic layers combined and dried over anhydrous sodium sulfate and concentrated. The residue was applied onto a silica gel column with ethyl acetate/petroleum ether (1:3-1:0). This resulted in 3.2 g (59.02%) of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitroso-4-[(oxan-4-ylmethyl)amino]benzenesulfonyl]carbamoyl)phenyl]piperazine-1-carboxylate hydrate as yellow solid.LC-MS: (ES, m/z): M+1:921.

Synthesis of 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide:Into a 100-mL round-bottom flask, was placed tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitro-4-[(oxan-4-ylmethyl)amino]benzenesulfonyl]carbamoyl)phenyl]piperazine-1-carboxylate (2.40 g, 2.605 mmol, 1.00 equiv), DCM (30.00 mL), TFA (10.00 mL),The resulting solution was stirred for 4 h at RT. The resulting mixture was concentrated. Added CH₃CN (30.00 mL), ethylenediamine (0.79 g, 13.145 mmol, 5.05 equiv). The resulting solution was allowed to react for an additional 6 h at 60 degrees C. The resulting mixture was concentrated. The crude product (3.0 g) was purified by Flash-Prep-HPLC with the following conditions (CombiFlash-1): Column, C18 silica gel; mobile phase, CH₃CN:H₂O(0.5% FA)=5% increasing to CH₃CN:H₂O(0.5% FA)=60 within 7 min; Detector, UV 254 nm. This resulted in 600 mg (33.34%) of 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide as yellow solid.LC-MS: (ES, m/z): M+1:691.

Synthesis of 2-bromo-1,4,4-trimethylcyclohex-1-ene hydrate: Into a 250-mL 3-necked round-bottom flask, was placed CHCl₃(100.00 mL), DMF (7.24 g, 99.050 mmol, 2.5 equiv). This was followed by the addition of PBr₃ (24.65 g, 91.065 mmol, 2.30 equiv) dropwise with stirring. The resulting solution was stirred for 1 hr at room temperature. Then to this was added cyclohexanone, 3,3-dimethyl-(5.00 g, 39.620 mmol, 1.00 equiv) dropwise with stirring at 0 degrees C. The resulting solution was allowed to react, for 12 hr at RT. The reaction was then pulled into 200 mL of water/ice. The pH value of the solution was adjusted to 5 with Sat.Na₂CO₃. The resulting solution was extracted with 3×100 mL of dichloromethane and the organic layers combined and dried over anhydrous sodium sulfate and concentrated. This resulted in 3.1 g (35.38%) of 2-bromo-1,4,4-trimethylcyclohex-1-ene hydrate as colorless oil.LC-MS: (ES, m/z): M+1:217/219.

Synthesis of 4,4-dimethyl-2-[4-(trifluoromethyl)phenyl]cyclohex-1-ene-1-carbaldehyde: Into a 40-mL vial purged and maintained with an inert atmosphere of nitrogen, was placed 2-bromo-4,4-dimethylcyclohex-1-ene-1-carbaldehyde (300.00 mg, 1.382 mmol, 1.00 equiv), 4-(trifluoromethyl)phenylboronic acid (313.30 mg, 1.650 mmol, 1.20 equiv), Pd(PPh₃)₂Cl₂ (48.49 mg, 0.069 mmol, 0.05 equiv), Na₂CO₃ (292.91 mg, 2.764 mmol, 2 equiv), DME (4.00 mL), H₂O (0.20 mL). The resulting solution was stirred for 12 hr at 100 degrees C. The reaction mixture was cooled to room temperature. The resulting mixture was concentrated. The residue was applied onto a silica gel column with ethyl acetate/petroleum ether (0:100-1:6). This resulted in 190 mg (48.71%) of 4,4-dimethyl-2-[4-(trifluoromethyl)phenyl]cyclohex-1-ene-1-carbaldehyde as colorless oil. LC-MS-PH-PHNW-4-175-1: (ES, m/z): M+1:283.

Synthesis of 4-[4-([4,4-dimethyl-2-[4-(trifluoromethyl)phenyl]cyclohex-1-en-1-yl]methyl)piperazin-1-yl]-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial, was placed 4,4-dimethyl-2-[4-(trifluoromethyl)phenyl]cyclohex-1-ene-1-carbaldehyde (57.00 mg, 0.2 mmol, 1.00 equiv), 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide (139.47 mg, 0.202 mmol, 1 equiv), MeOH (1.50 mL), DCM (1.50 mL), ZnCl₂ (55.05 mg, 0.404 mmol, 2 equiv), NaBH₃CN (25.38 mg, 0.404 mmol, 2 equiv). The resulting solution was stirred for 5 h at 60 degrees C. The reaction mixture was cooled to room temperature. The resulting mixture was concentrated. The residue was applied onto Pre-TLC with dichloromethane/methanol (95:5). The crude product (45 mg) was purified by Flash-Prep-HPLC with the following conditions (IntelFlash-1): Column, C18 silica gel; mobile phase, CH₃CN: H₂O(0.5% NH₃H₂O)=10% increasing to CH₃CN:H₂O(0.5% NH₃H₂O)=60 within 7 min; Detector, UV 254 nm. This resulted in 4.1 mg (2.12%) of 4-[4-([4,4-dimethyl-2-[4-(trifluoromethyl)phenyl]cyclohex-1-en-1-yl]methyl)piperazin-1-yl]-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as yellow solid.LC-MS (ES, m/z): M+1:957. ¹H NMR (300 MHz, DMSO-d₆, ppm) δ12.48 (s, 1H), 8.74-8.61 (m, 2H), 8.46 (d, J=5.4 Hz, 1H), 8.18-8.06 (m, 2H), 7.79 (d, J=9.7 Hz, 1H), 7.55 (d, J=7.8 Hz, 2H), 7.22-7.06 (m, 2H), 7.02-6.89 (m, 2H), 6.67 (d, J=9.3 Hz, 1H), 6.55 (s, 1H), 6.11 (s, 1H), 3.59-3.35 (m, 4H), 3.35-3.06 (m, 4H), 2.81 (s, 2H), 2.46-2.16 (m, 4H), 2.12-1.86 (m, 3H)1.68 (dd, J=38.2, 9.6 Hz, 4H), 1.57-1.39 (m, 4H), 0.99 (s, 6H), 0.96-0.78 (m, 8H).

### Compound 4-4: Preparation of 4-(4-[[2-(4-chlorophenyl)-4-(trifluoromethyl)cyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide

Synthesis of (E)-2-(2-ethoxyvinyl)-4,4,5,5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil. LC-MS: (ES, m/z): M+1=199; H-NMR (300 MHz, DMSO-d₆, ppm) δ6.91-6.95 (m, 1H), 4.30-4.34 (m, 1H), 3.78-3.82 (m, 2H), 1.14-1.24 (m, 15H).

Synthesis of N-[(2R)-2-hydroxypropyl]acetamide: Into a 250-mL 3-necked round-bottom flask purged and maintained with an inert atmosphere of nitrogen, was placed (2R)-1-aminopropan-2-ol (100 g, 1.3 mol, 1 equiv), DCM (1 L), TEA (160 g, 1.6 mol, 1.2 equiv). This was followed by the addition of a solution of acetyl acetate (136 g, 1.3 mol, 1.0 equiv) in 100 ml (DCM) dropwise with stirring at 0-10° C. The resulting solution was stirred for 14 hr at room temperature. The resulting mixture was concentrated. The residue was applied onto a silica gel column with dichloromethane/methanol (100:5). This resulted in 250 g (crude) of N-[(2R)-2-hydroxypropyl]acetamide as a yellow oil.

Synthesis of 5-bromo-6-fluoropyridin-2-amine: Into a 20L 4-necked round-bottom flask, was placed 6-fluoropyridin-2-amine (4500.00 g, 40.178 mol, 1.00 equiv), ACN (20.00 L). This was followed by the addition of NBS (7035.17 g, 41.383 mol, 1.03 equiv) in portions at R.T (25) degrees C. The resulting solution was stirred for 2 h at room temperature. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as a light brown solid. LC-MS: (ES, m/z): M+1=190; H-NMR (300 MHz, DMSO-d₆, ppm) δ7.63-7.71 (m, 1H), 6.56 (s, 2H), 6.30-631 (m, 1H).

Synthesis of 5-bromo-6-fluoro-3-iodopyridin-2-amine: Into a 20L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 2h at room temperature. The resulting solution was diluted with 100 L of water. The mixture was filtrated, collection of filter cake and the filter cake was washed by water (35L×2) and dried by oven to give product 9840 g (Y=90%) of 5-bromo-6-fluoro-3-iodopyridin-2-amine as a brown solid. LC-MS: (ES, m/z): M+1=317; H-NMR (300 MHz, DMSO-d₆, ppm) δ8.16-8.23 (m, 1H), 6.69 (s, 2H).

Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature in a liquid nitrogen bath. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil. LC-MS: (ES, m/z) M+1=261; H-NMR (300 MHz, DMSO-d₆, ppm) δ7.96-7.99 (m, 1H), 6.72-6.76 (m, 1H), 5.49-5.53 (m, 1H), 3.99-4.06 (m, 3H).

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as a light brown solid. LC-MS: (ES, m/z): M+1=215; H-NMR (300 MHz, DMSO-d₆, ppm) δ9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS (ES, m/z) M+1=345; H-NMR (300 MHz, DMSO-d₆, ppm) δ8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

### Synthesis of (R)-N-(2-(5-bromo-1-((2-(trimethylsilyl)ethoxy)methyl)-1H-pyrr olo[2,3-b]pyidin-6-yloxy)propyl)acetamide: Into a 50-mL round-bottom flask, was placed N-[(2R)-2-hydroxypropyl] acetamide (crude, 81.6 g, 697 mmol, 3.00 equiv), dioxane (800 ml). This was followed by the addition of NaH (28 g, 697 mmol, 3 equiv), in portions at 5° C. 15° C. The resulting solution was stirred for 30 min at RT. To this was added a solution of 5-bromo-6-fluoro-1-((2-(trimethylsilyl)ethoxy)methyl)-1H-pyrrolo[2,3-b] pyridine (80 g, 232 mmol, 1 equiv) in dioxane (100 ml). The resulting solution was stirred for 4 hr at 80° C. in an oil bath. The reaction was then quenched by the addition of 50 ml of water at 10° C. The resulting solution was concentrated and diluted with 500 ml H₂O. The resulting solution was extracted with 3×500 ml of ethyl acetate. The resulting mixture was washed with 1×300 ml of NaCl (aq). The mixture was dried over anhydrous sodium sulfate and concentrated. The residue was applied onto a silica gel column with PE/EA (100:20). This resulted in 40 g (pure) and 30 g (70%) of (R)-N-(2-(5-bromo-1-((2-(trimethylsilyl)ethoxy)methyl)-1H-pyrrolo[2,3-b]pyridin-6-yloxy)propyl)acetamide as a yellow oil

Synthesis of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one: Into a 1-mL round-bottom flask purged and maintained with an inert atmosphere of nitrogen, was placed (R)-N-(2-(5-bromo-1-((2-(trimethylsilyl)ethoxy)methyl)-1H-pyrrolo[2,3-b]pyridin-6-yloxy)propyl)acetamide (40 g, 90.7 mmol, 1 equiv), dioxane (500 mL), Cs₂CO₃ (88 g, 272 mmol, 3 equiv), BrettPhos Pd G3 (4.1 g, 4.5 mmol, 0.05 equiv). The resulting solution was concentrated and diluted with 300 ml H₂O. The resulting solution was extracted with 3×300 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (40:100). This resulted in 33 g(crude, 70%) of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one as brown oil.

Synthesis of (12R)-4-(trimethylsilyl)ethoxy)methyl)-12-methyl-13-oxa-2,4,10-triazatricyclo[7.4.0.0\hat{ }[3,7]]trideca-1(9),2,5,7-tetraene: Into a 1-L vial, was placed1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one (33 g), methanol (300 mL), sodium hydroxide (2M, 300 ml). The resulting solution was stirred for 6 hr at 80° C. in an oil bath. v). The resulting solution was concentrated(MeOH) and extracted with 3×200 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (1:1). This resulted in 16.2 g of (12R)-4-(trimethylsilyl)ethoxy)methyl)-12-methyl-13-oxa-2,4,10-triazatricyclo [7.4.0.0\hat{ }[3,7]]trideca-1(9),2,5,7-tetraene as brown oil.

### Synthesis of 2-bromo-4-[4-(tert-butoxycarbonyl)piperazin-1-yl]benzoic acid: Into a 100-mL 3-necked round-bottom flask, was placed tert-butyl 4-[3-bromo-4-(methoxycarbonyl)phenyl]piperazine-1-carboxylate (4.00 g, 10.0 mmol, 1.00 equiv,), MeOH(20 ml), THF(20 ml), H₂O (20 ml), NaOH (1.60 g, 40.0 mmol, 4.00 equiv,). The resulting solution was stirred for 3 h at 30 degrees C. The resulting solution was diluted with 40 mL of H₂O. The pH value of the solution was adjusted to 5 with HCl (0.5 mol/L). The resulting solution was extracted with 3×30 mL of ethyl acetateand the organic layers combined. The resulting mixture was washed with 1×50 mL of brine. The mixture was dried over anhydrous sodium sulfate and concentrated. This resulted in 3.8 g (98.45%) of 2-bromo-4-[4-(tert-butoxycarbonyl)piperazin-1-yl]benzoic acid as a white solid. LC-MS (ES, m/z): M+1: 385 Synthesis of tert-butyl 4-[3-bromo-4-([3-nitroso-4-[(oxan-4-ylmethyl)amino]benzenesulfonyl]carbamoyl)phenyl]piperazine-1-carboxylate hydrate: Into a 100-mL 3-necked round-bottom flask, was placed 2-bromo-4-[4-(tert-butoxycarbonyl)piperazin-1-yl]benzoic acid (3.80 g, 9.864 mmol, 1.00 equiv), 3-nitro-4-[(oxan-4-ylmethyl)amino]benzenesulfonamide (3.11 g, 9.864 mmol, 1 equiv), EDCI (2.27 g, 11.836 mmol, 1.2 equiv), DMAP (2.41 g, 19.727 mmol, 2 equiv), DCM (40.00 mL). The resulting solution was stirred for 12 h at 30 degrees C. The resulting solution was diluted with 50 mL of H₂O. The resulting solution was extracted with 3×50 mL of dichloromethane and the organic layers combined. The resulting mixture was washed with 1×50 mL of brine. The mixture was dried over anhydrous sodium sulfate and concentrated. The residue was applied onto a silica gel column with dichloromethane/methanol (90:10). This resulted in 5.4 g (79.73%) of tert-butyl 4-[3-bromo-4-([3-nitroso-4-[(oxan-4-ylmethyl)amino]benzenesulfonyl]carbamoyl)phenyl]piperazine-1-carboxylate hydrate as yellow solid. LC-MS (ES, m/z): M+1:682

Synthesis of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitroso-4-[(oxan-4-ylmethyl)amino]benzenesulfonyl]carbamoyl)phenyl]piperazine-1-carboxylate hydrate: Into a 100-mL round-bottom flask purged and maintained with an inert atmosphere of nitrogen, was placed tert-butyl 4-[3-bromo-4-([3-nitro-4-[(oxan-4-ylmethyl)amino]benzenesulfonyl]carbamoyl)phenyl]piperazine-1-carboxylate (4.00 g, 5.860 mmol, 1.00 equiv), (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (1.87 g, 5.860 mmol, 1.00 equiv), DMF (60.00 mL), CuI (0.22 g, 1.172 mmol, 0.20 equiv), Cs₂CO₃ (3.82 g, 11.720 mmol, 2.00 equiv), N1,N2-bis(4-hydroxy-2,6-dimethylphenyl)oxalamide (0.58 g, 1.766 mmol, 0.30 equiv). The resulting solution was stirred for 2 h at 100 degrees C. The reaction mixture was cooled to room temperature with a water bath. The resulting solution was diluted with 100 mL of H₂O. The resulting solution was extracted with 3×100 mL of ethyl acetate and the organic layers combined and dried over anhydrous sodium sulfate and concentrated. The residue was applied onto a silica gel column with ethyl acetate/petroleum ether (1:3-1:0). This resulted in 3.2 g (59.02%) of tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitroso-4-[(oxan-4-ylmethyl)amino]benzenesulfonyl]carbamoyl)phenyl]piperazine-1-carboxylate hydrate as yellow solid. LC-MS (ES, m/z): M+1:921

### Synthesis of 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide: Into a 100-mL round-bottom flask, was placed tert-butyl 4-[3-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-4-([3-nitro-4-[(oxan-4-ylmethyl)amino]benzenesulfonyl]carbamoyl)phenyl]piperazine-1-carboxylate (2.40 g, 2.605 mmol, 1.00 equiv), DCM (30.00 mL), TFA (10.00 mL),The resulting solution was stirred for 4 h at RT. The resulting mixture was concentrated. Added CH₃CN (30.00 mL), ethylenediamine (0.79 g, 13.145 mmol, 5.05 equiv). The resulting solution was allowed to react for an additional 6 h at 60 degrees C. The resulting mixture was concentrated. The crude product (3.0 g) was purified by Flash-Prep-HPLC with the following conditions (CombiFlash-1): Column, C18 reversed phase column; mobile phase, CH₃CN:H₂O (0.5% FA)=5% increasing to 60% within 7 min; Detector, UV 254 nm. This resulted in 600 mg (33.34%) of 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide as yellow solid. LC-MS: (ES, m/z): M+1:691

Synthesis of 2-bromo-4-(trifluoromethyl)cyclohex-1-ene-1-carbaldehyde: Into a 250-mL 3-necked round-bottom flask, was placed CHCl₃ (40 mL). This was followed by the addition of DMF (4.49 g, 61.428 mmol, 6.00 equiv) dropwise with stirring at 15-20 degrees C. To this was added PBr₃ (14.27 g, 52.718 mmol, 5.15 equiv) dropwise with stirring at 15-20 degrees C. After addition, the mixture was stirred for 1 h. To the mixture was added a solution of 3-(trifluoromethyl)cyclohexan-1-one (1.70 g, 10.232 mmol, 1.00 equiv) in CHCl₃ (20 mL) dropwise with stirring at room temperature. The resulting solution was stirred overnight at room temperature. The resulting solution was poured into 300 mL of water. The pH value of the solution was adjusted to 5 with aqueous NaHCO₃. The resulting solution was extracted with 3×100 mL of dichloromethane and the organic layers combined. The resulting mixture was washed with 1×500 ml of brine. The mixture was dried over anhydrous sodium sulfate and concentrated under vacuum. The residue was applied onto a silica gel column with ethyl acetate/petroleum ether (1:10). This resulted in 1.07 g (40.68%) of 2-bromo-4-(trifluoromethyl)cyclohex-1-ene-1-carbaldehyde as yellow oil. H-NMR (300 MHz, Chloroform-d, ppm) δ10.04 (s, 1H), 2.98-2.87 (m, 2H), 2.75-2.63 (m, 1H), 2.59-2.45 (m, 1H), 2.26-2.15 (m, 2H), 1.64-1.54 (m, 1H).

Synthesis of 2-(4-chlorophenyl)-4-(trifluoromethyl)cyclohex-1-ene-1-carbaldehyde: Into a 40-mL vial purged and maintained with an inert atmosphere of nitrogen, was placed 2-bromo-4-(trifluoromethyl)cyclohex-1-ene-1-carbaldehyde (1.07 g, 4.163 mmol, 1.00 equiv), DME (10.00 mL), H₂O (0.50 mL), benzeneboronic acid, p-chloro-(782.00 mg, 5.001 mmol, 1.20 equiv), Na₂CO₃ (886.00 mg, 8.359 mmol, 2.01 equiv), Pd(PPh₃)₂Cl₂ (147.00 mg, 0.209 mmol, 0.05 equiv). The resulting solution was stirred overnight at 60 degrees C. in an oil bath. The reaction mixture was cooled to room temperature. The resulting mixture was concentrated under vacuum. The residue was applied onto a silica gel column with ethyl acetate/petroleum ether (1:7). This resulted in 893 mg (74.31%) of 2-(4-chlorophenyl)-4-(trifluoromethyl)cyclohex-1-ene-1-carbaldehyde as a light yellow solid. H-NMR (300 MHz, Chloroform-d, ppm) δ9.51 (s, 1H), 7.44-7.40 (m, 2H), 7.24-7.20 (m, 2H), 2.86-2.73 (m, 2H), 2.69-2.62 (m, 1H), 2.26-2.15 (m, 2H), 1.73-1.50 (m, 2H).

Synthesis of 4-(4-[[2-(4-chlorophenyl)-4-(trifluoromethyl)cyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial, was placed 2-(4-chlorophenyl)-4-(trifluoromethyl)cyclohex-1-ene-1-carbaldehyde (12.50 mg, 0.043 mmol, 1.00 equiv), 2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]-4-(piperazin-1-yl)benzamide (30.00 mg, 0.043 mmol, 1.00 equiv), MeOH (3.00 mL), ZnCl₂ (11.70 mg, 0.086 mmol, 1.98 equiv), NaBH₃CN (5.50 mg, 0.088 mmol, 2.02 equiv). The resulting solution was stirred for 2 hr at 80 degrees C. in an oil bath. The reaction mixture was cooled to room temperature. The resulting solution was poured into 100 mL of water. The resulting solution was extracted with 3×30 mL of dichloromethane and the organic layers combined. The resulting mixture was washed with 1×200 ml of brine. The mixture was dried over anhydrous sodium sulfate and concentrated under vacuum. The residue was applied onto a silica gel column with dichloromethane/methanol (20:1). The crude product was purified by Prep-HPLC with the following conditions: Column, XBridge Shield RP18 OBD Column, Sum, 19*150 mm; mobile phase, Water (0.05% NH₃.H₂O) and ACN (27% Phase B up to 50% in 7 min); Detector, UV 254/220 nm. This resulted in 5 mg (11.99%) of 4-(4-[[2-(4-chlorophenyl)-4-(trifluoromethyl)cyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as a yellow solid. LC-MS (ES, m/z): M+1: 963; H-NMR (300 MHz, DMSO-d₆, ppm) δ12.10 (s, 1H), 10.99 (s, 1H), 8.52 (s, 1H), 8.37 (s, 1H), 7.56 (d, J=8.6 Hz, 1H), 7.46-7.34 (m, 2H), 7.25-7.09 (m, 2H), 7.03 (s, 1H), 6.77 (d, J=26.6 Hz, 3H), 5.97 (s, 1H), 3.87 (dd, J=11.1, 4.1 Hz, 2H), 3.29-3.06 (m, 6H), 2.85-2.63 (m, 3H), 2.45-2.12 (m, 7H), 2.00 (q, J=5.9, 4.6 Hz, 2H), 1.90 (d, J=9.2 Hz, 1H), 1.63 (d, J=13.0 Hz, 2H), 1.57-1.44 (m, 1H), 1.41-1.10 (m, 9H).

### Compound 4-5: Preparation of 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-N-[3-nitro-4-[(oxan-4-ylmethyl)amino]benzenesulfonyl]benzamide (assumed

Synthesis of N-[(trans)(3S,4R)-4-hydroxyoxolan-3-yl]-4-methylbenzenesulfonamide: Into a 250-mL round-bottom flask purged and maintained with an inert atmosphere of nitrogen, was placed 3,6-dioxabicyclo[3.1.0]hexane (5.00 g, 58.079 mmol, 1.00 equiv), dioxane (100.00 mL), p-toluenesulfonamide (11.93 g, 69.681 mmol, 1.20 equiv), TEBAC (1.33 g, 5.833 mmol, 0.10 equiv), K₂CO₃ (0.80 g, 5.788 mmol, 0.10 equiv). The resulting solution was stirred for 3 days at 90 degrees C. in an oil bath. The reaction mixture was cooled to room temperature. The solids were filtered out. The resulting mixture was concentrated under vacuum. The residue was applied onto a silica gel column with ethyl acetate/petroleum ether (2:1). This resulted in 10.69 g (25.75%) of N-[(trans)(3S,4R)-4-hydroxyoxolan-3-yl]-4-methylbenzenesulfonamide as a white solid. LC-MS: (ES, m/z): M+1=258.

Synthesis of N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide: Into a 250-mL round-bottom flask purged and maintained with an inert atmosphere of nitrogen, was placed N-[(trans)(3S,4R)-4-hydroxyoxolan-3-yl]-4-methylbenzenesulfonamide (10.50 g, 14.691 mmol, 1.30 equiv, 36%), THE (100 mL). This was followed by the addition of NaH (2.71 g, 67.756 mmol, 6.01 equiv, 60%), in portions at 0 degrees C. The resulting solution was stirred for 30 min at 0 degrees C. To this was added 5-bromo-6-fluoro-1-[[2-(trimethylsilyl)ethoxy]methyl]pyrrolo[2,3-b]pyridine (3.89 g, 11.266 mmol, 1.00 equiv) at 0 degrees C. The resulting solution was stirred for 4 hr at 70 degrees C. in an oil bath. The reaction mixture was cooled to room temperature. The reaction was then quenched by the addition of 500 mL of aqueous NH₄Cl. The resulting solution was extracted with 3×300 mL of ethyl acetate and the organic layers combined. The resulting mixture was washed with 1×1500 mL of brine. The mixture was dried over anhydrous sodium sulfate and concentrated under vacuum. The residue was applied onto a silica gel column with ethyl acetate/petroleum ether (1:2). This resulted in 3 g (45.71%) of N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide as light yellow oil. ¹H NMR (300 MHz, Chloroform-d, ppm) δ8.04 (s, 1H), 7.78-7.61 (m, 2H), 7.25-7.10 (m, 3H), 6.43 (d, J=3.6 Hz, 1H), 5.67 (d, J=10.8 Hz, 1H), 5.60 (d, J=5.7 Hz, 1H), 5.49 (d, J=10.8 Hz, 1H), 5.40 (dt, J=6.1, 3.1 Hz, 1H), 4.27 (dd, J=10.5, 5.9 Hz, 1H), 4.21-4.03 (m, 1H), 4.03-3.86 (m, 2H), 3.70-3.52 (m, 3H), 2.32 (s, 3H), 0.93 (ddt, J=10.6, 5.5, 2.6 Hz, 2H),-0.02 (s, 9H).

Synthesis of (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene: Into a 250-mL round-bottom flask purged and maintained with an inert atmosphere of nitrogen, was placed N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide (3.00 g, 5.149 mmol, 1.00 equiv), DMF (50.00 mL), phen (743.00 mg, 4.123 mmol, 0.80 equiv), CuI (785.00 mg, 4.122 mmol, 0.80 equiv), K₂CO₃ (2.14 g, 15.484 mmol, 3.01 equiv). The resulting solution was stirred for 2 days at 120 degrees C. in an oil bath. The reaction mixture was cooled to room temperature. The resulting solution was diluted with 500 mL of water. The resulting solution was extracted with 3×200 mL of ethyl acetate and the organic layers combined. The resulting mixture was washed with 1×1000 mL of brine. The mixture was dried over anhydrous sodium sulfate and concentrated under vacuum. The residue was applied onto a silica gel column with ethyl acetate/petroleum ether (1:2). This resulted in 2.5 g (82.26%) of (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene as yellow oil. LC-MS: (ES, m/z): M+1=502.

Synthesis of (11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) and (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed): Into a 100-mL round-bottom flask, was placed Mg (2.04 g, 83.933 mmol, 19.81 equiv), MeOH (30.00 mL), (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (2.50 g, 4.236 mmol, 1.00 equiv, 85%). The resulting solution was stirred for 2 hr at 60 degrees C. in an oil bath. The reaction mixture was cooled to room temperature. The resulting solution was diluted with 300/300 mL of NaHCO₃and CH₂Cl2. The solids were filtered out and the organic was separated. The mixture was dried over anhydrous sodium sulfate and concentrated under vacuum. The residue was applied onto a silica gel column with ethyl acetate/petroleum ether (1:3). The crude product was purified by Chiral-Prep-HPLC with the following conditions: Mobile phase: A:n-Hexane (0.1% DEA) B:ETOH; Flow rate: 20 mL/min; Column: DAICEL CHIRALPAK IA, 250*20 mm, Sum; Gradient:12% B in 20 min; 220 nm. This resulted in 350 mg (23.78%) of (11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) as yellow oil. and 400 mg (27.18%) of (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) as yellow oil. Peak 1: LC-MS: (ES, m/z): M+1=348, R,T=1.211 min. ¹H NMR (300 MHz, Chloroform-d, ppm) δ7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H),-0.04 (s, 9H). Peak 2: LC-MS: (ES, m/z): M+1=348, R,T=1.211 min. ¹H NMR (300 MHz, Chloroform-d, ppm) δ7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H),-0.04 (s, 9H).

Synthesis of methyl 2-bromo-4-(4-[[2-(4-chlorophenyl)-4,4-dimethylcyclohex-1-en-1-yl]methyl]piperazin-1-yl)benzoate: Into a 250-mL round-bottom flask, was placed a solution of 1-[[2-(4-chlorophenyl)-4,4-dimethylcyclohex-1-en-1-yl]methyl]piperazine (15.09 g, 47.32 mmol, 1.00 equiv) in DMA (150 mL), DIEA (12.9 g, 99.81 mmol, 2.00 equiv), methyl 2-bromo-4-fluorobenzoate (11.6 g, 49.78 mmol, 1.00 equiv). The resulting solution was stirred for 12 h at 100 degree. The reaction mixture was cooled to room temperature. The reaction was then quenched by the addition of 50 mL of water. The resulting solution was extracted with 3×100 mL of ethyl acetate and the organic layers combined. The resulting mixture was washed with 3×100 mL of brine. The mixture was dried over anhydrous sodium sulfate, then filtered and concentrated under vacuum. The residue was applied onto a silica gel column with ethyl acetate/petroleum ether (0:1-1:5). This resulted in 7 g (crude) of methyl 2-bromo-4-(4-[[2-(4-chlorophenyl)-4,4-dimethylcyclohex-1-en-1-yl]methyl]piperazin-1-yl)benzoate as yellow oil. LC-MS: (ES, m/z): M+1=533, 531.

Synthesis of methyl 4-(4-[[2-(4-chlorophenyl)-4,4-dimethylcyclohex-1-en-1-yl]methyl]piperazin-1-yl)-2-[(11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed): Into a 40-mL vial purged and maintained with an inert atmosphere of nitrogen, was placed (11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraene (assumed) (350.00 mg, 1.007 mmol, 1.00 equiv), toluene (15.00 mL), methyl 2-bromo-4-(4-[[2-(4-chlorophenyl)-4,4-dimethylcyclohex-1-en-1-yl]methyl]piperazin-1-yl)benzoate (1.07 g, 2.012 mmol, 2.00 equiv), Pd₂(dba)3.CHCl₃ (208.00 mg, 0.201 mmol, 0.20 equiv), Xantphos (234.00 mg, 0.404 mmol, 0.40 equiv), Cs₂CO₃ (985.00 mg, 3.023 mmol, 3.00 equiv). The resulting solution was stirred for 3 hr at 100 degrees C. in an oil bath. The reaction mixture was cooled to room temperature. The resulting mixture was concentrated under vacuum. The residue was applied onto a silica gel column with ethyl acetate/petroleum ether (1:2). This resulted in 720 mg (89.52%) of methyl 4-(4-[[2-(4-chlorophenyl)-4,4-dimethylcyclohex-1-en-1-yl]methyl]piperazin-1-yl)-2-[(11S,15R)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) as a yellow solid. LC-MS: (ES, m/z): M+1=798.

Synthesis of methyl 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed): Into a 40-mL vial, was placed methyl 4-(4-[[2-(4-chlorophenyl)-4,4-dimethylcyclohex-1-en-1-yl]methyl]piperazin-1-yl)-2-[(11R,15S)-4-[[2-(trimethylsilyl)ethoxy]methyl]-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) (720.00 mg, 0.902 mmol, 1.00 equiv), 1.0 M TBAF/THF (15.00 mL), ethylenediamine (1.30 g, 21.631 mmol, 23.99 equiv). The resulting solution was stirred for 8 hr at 70 degrees C. in an oil bath. The reaction mixture was cooled to room temperature. The resulting mixture was concentrated under vacuum. The residue was applied onto a silica gel column with ethyl acetate/petroleum ether (2:1). This resulted in 350 mg (58.09%) of methyl 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) as a light yellow solid. LC-MS: (ES, m/z): M+1=668. Synthesis of 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed): Into a 40-mL vial, was placed methyl 4-(4-[[2-(4-chlorophenyl)-4,4-dimethylcyclohex-1-en-1-yl]methyl]piperazin-1-yl)-2-[(11R,15S)-13,16-dioxa-2,4,10-triazatetracyclo[7.7.0.0\hat{ }[3,7]0.0\hat{ }[11,15]]hexadeca-1(9),2,5,7-tetraen-10-yl]benzoate (assumed) (150.00 mg, 0.224 mmol, 1.00 equiv), dioxane (3.00 mL), MeOH (3.00 mL), NaOH (0.60 mL, 2.400 mmol, 10.69 equiv). The resulting solution was stirred for 4 hr at 70 degrees C. in an oil bath. The reaction mixture was cooled to room temperature. The resulting mixture was concentrated under vacuum. The pH value of the solution was adjusted to 5-6 with HCl (2 mol/L). The resulting solution was extracted with 3×50 mL of ethyl acetate and the organic layers combined. The resulting mixture was washed with 1×300 mL of brine. The mixture was dried over anhydrous sodium sulfate and concentrated under vacuum. The residue was purified by Prep-TLC with dichloromethane/methanol (10:1). This resulted in 80 mg (54.48%) of 4-(4-[[2-(4-chlorophenyl)-4,4-dimethylcyclohex-1-en-1-yl]methyl]piperazin-1-yl)-2-[(11 S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (assumed) as a white solid. LC-MS: (ES, m/z): M+1=654.

Synthesis of 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]-N-[3-nitro-4-[(oxan-4-ylmethyl)amino]benzenesulfonyl]benzamide (assumed): Into a 40-mL vial, was placed 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7]0.0\hat{ }[11,15]]hexadeca-1(9),2,5,7-tetraen-10-yl]benzoic acid (45.00 mg, 0.069 mmol, 1.00 equiv), DCM (5.00 mL), 3-nitro-4-[(oxan-4-ylmethyl)amino]benzenesulfonamide (assumed) (22.00 mg, 0.070 mmol, 1.01 equiv), EDCI (26.00 mg, 0.136 mmol, 1.97 equiv), DMAP (34.00 mg, 0.278 mmol, 4.05 equiv). The resulting solution was stirred overnight at 25 degrees C. The resulting mixture was concentrated under vacuum. The crude product was purified by Prep-HPLC with the following conditions (Prep-HPLC-006): Column, XBridge Shield RP18 OBD Column, Sum, 19*150 mm; mobile phase, Water (0.05% NH₃.H₂O) and ACN (40% Phase B up to 70% in 7 min); Detector, UV 254/220 nm. This resulted in 30 mg (45.84%) of 4-(4-[[2-(4-chlorophenyl)-4,4-dimethylcyclohex-1-en-1-yl]methyl]piperazin-1-yl)-2-[(11S,15R)-13,16-dioxa-2,4,10-triazatetracyclo[7.7.0.0\hat{ }[3,7].0 \hat{ }[11,15]]hexadeca-1(9),2,5,7-tetraen-10-yl]-N-[3-nitro-4-[(oxan-4-ylmethyl)amino]benzenesulfonyl]benzamide (assumed) as a yellow solid. LC-MS: (ES, m/z): M+1=951. ¹H NMR (300 MHz, DMSO-d₆,ppm) δ11.06 (d, J=34.5 Hz, 1H), 8.48-8.26 (m, 2H), 7.64-6.99 (m, 7H), 6.89-6.51 (m, 4H), 6.08-5.96 (m, 1H), 4.46 (dd, J=68.8, 60.0 Hz, 2H), 3.94-3.41 (m, 6H), 3.13 (d, J=20.7 Hz, 8H), 2.75 (d, J=10.9 Hz, 2H), 2.23 (s, 6H), 1.98 (s, 2H), 1.88 (s, 1H), 1.64 (d, J=13.1 Hz, 2H), 1.41 (s, 2H), 1.27 (d, J=13.8 Hz, 2H), 0.95 (s, 6H).

### Compound 4-6: Preparation of 4-(4-[[4-(4-chlorophenyl)-6,6-dimethyl-2,5-dihydropyran-3-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide

Synthesis of (E)-2-(2-ethoxyvinyl)-4,4,5,5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil. LC-MS: (ES, m/z): M+1=199. ¹H-NMR: (300 MHz, DMSO-d₆, ppm): δ6.91-6.95 (m, 1H), 4.30-4.34 (m, 1H), 3.78-3.82 (m, 2H), 1.14-1.24 (m, 15H).

Synthesis of N-[(2R)-2-hydroxypropyl] acetamide: Into a 3L 3-necked round-bottom flask purged and maintained with an inert atmosphere of nitrogen, was placed (2R)-1-aminopropan-2-ol (100 g, 1.3 mol, 1 equiv), DCM (1 L), TEA (160 g, 1.6 mol, 1.2 equiv). This was followed by the addition of a solution of acetyl acetate (136 g, 1.3 mol, 1.0 equiv) in 100 ml DCM dropwise with stirring at 0-10° C. The resulting solution was stirred for 14 h at room temperature. The resulting mixture was concentrated. The residue was applied onto a silica gel column with dichloromethane/methanol (100:5). This resulted in 250 g (crude) of N-[(2R)-2-hydroxypropyl]acetamide as yellow oil.

Synthesis of 5-bromo-6-fluoropyridin-2-amine: Into a 50 L 4-necked round-bottom flask, was placed 6-fluoropyridin-2-amine (4500.00 g, 40.178 mol, 1.00 equiv), ACN (10.00 L). This was followed by the addition of NBS (7035.17 g, 41.383 mol, 1.03 equiv) in portions at R.T (25) degrees C. The resulting solution was stirred for 3 h at RT. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as light brown solid. LC-MS: (ES, m/z): M+1=190. ¹H-NMR: (300 MHz, DMSO-d₆, ppm): δ7.63-7.71 (m, 1H), 6.56 (s, 2H), 6.30-631 (m, 1H).

Synthesis of 5-bromo-6-fluoro-3-iodopyridin-2-amine: Into a 50L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 3 h at room temperature. The resulting solution was diluted with 100 L of water. The mixture was filtrated, collection of filter cake and the filter cake was washed by water (35L×2) and dried by oven to give product 9840 g(Y=90%) of 5-bromo-6-fluoro-3-iodopyridin-2-amine as a brown solid. LC-MS: (ES, m/z): M+1=317, 319, R.T=1.087 min. ¹H-NMR: (300 MHz, DMSO-d₆, ppm): δ8.16-8.23 (m, 1H), 6.69 (s, 2H).

Synthesis of 5-bromo-3-(2-ethoxyvinyl)-6-fluoropyridin-2-amine: Into a 20 L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil. LC-MS: (ES, m/z): M+1=261, 263. ¹H-NMR: (300 MHz, DMSO-d₆, ppm): δ7.96-7.99 (m, 1H), 6.72-6.76 (m, 1H), 5.49-5.53 (m, 1H), 3.99-4.06 (m, 3H).

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as light brown solid. LC-MS: (ES, m/z): M+1=215, 217. ¹H-NMR: (300 MHz, DMSO-d₆, ppm): δ9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS: (ES, m/z): M+1=345, 347, R.T=1.435 min. ¹H-NMR: (300 MHz, DMSO-d₆, ppm): δ8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

### Synthesis of (R)-N-(2-(5-bromo-1-((2-(trimethylsilyl)ethoxy)methyl)-1H-pyrr olo[2,3-b]pyidin-6-yloxy)propyl)acetamide: Into a 3 L round-bottom flask, was placed N-[(2R)-2-hydroxypropyl] acetamide (crude, 81.6 g, 697 mmol, 3.00 equiv), dioxane (800 ml). This was followed by the addition of NaH (28 g, 697 mmol, 3 equiv), in portions at 5° C.-15° C. The resulting solution was stirred for 30 min at RT. To this was added a solution of 5-bromo-6-fluoro-1-((2-(trimethylsilyl)ethoxy)methyl)-1H-pyrrolo[2,3-b] pyridine (80 g, 232 mmol, 1 equiv) in dioxane (100 ml). The resulting solution was stirred for 4 h at 80° C. in an oil bath. The reaction was then quenched by the addition of 50 ml of water at 10° C. The resulting solution was concentrated and diluted with 500 ml H₂O. The resulting solution was extracted with 3×500 ml of ethyl acetate. The resulting mixture was washed with 1×300 ml of NaCl (aq). The mixture was dried over anhydrous sodium sulfate and concentrated. The residue was applied onto a silica gel column with PE/EA (100:20). This resulted in 40 g (pure) and 30 g (70%) of (R)-N-(2-(5-bromo-1-((2-(trimethylsilyl)ethoxy)methyl)-1H-pyrrolo[2,3-b]pyridin-6-yloxy)propyl)acetamide as yellow oil

Synthesis of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one: Into a 1-L round-bottom flask purged and maintained with an inert atmosphere of nitrogen, was placed (R)-N-(2-(5-bromo-1-((2-(trimethylsilyl)ethoxy)methyl)-1H-pyrrolo[2,3-b]pyridin-6-yloxy)propyl)acetamide (40 g, 90.7 mmol, 1 equiv), dioxane (500 mL), Cs₂CO₃ (88 g, 272 mmol, 3 equiv), BrettPhos Pd G3 (4.1 g, 4.5 mmol, 0.05 equiv). The resulting solution was stirred for 14 h at 80° C. in an oil bath. The resulting solution was concentrated and diluted with 300 ml H₂O. The resulting solution was extracted with 3×300 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (40:100). This resulted in 33 g(crude, 70%) of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one as brown oil.

Synthesis of (12R)-4-(trimethylsilyl)ethoxy)methyl)-12-methyl-13-oxa-2,4,10-triazatricyclo[7.4.0.0\hat{ }[3,7]]trideca-1(9),2,5,7-tetraene: Into a 1L 3-necked round-bottom flask, was placed1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one (33 g), methanol (300 mL), sodium hydroxide (2M, 300 ml). The resulting solution was stirred for 14 h at 80° C. in an oil bath. The resulting solution was concentrated (MeOH) and extracted with 3×200 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (1:1). This resulted in 16.2 g of (12R)-4-(trimethylsilyl)ethoxy)methyl)-12-methyl-13-oxa-2,4,10-triazatricyclo [7.4.0.0\hat{ }[3,7]]trideca-1(9),2,5,7-tetraene as brown oil.

Synthesis of 4-bromo-6,6-dimethyl-2,5-dihydropyran-3-carbaldehyde: Into a 250-mL 3-necked round-bottom flask, was added CHCl₃ (80.00 mL), DMF (7.13 g, 97.5 mmol, 2.5 equiv). This was followed by the addition of phosphorus tribromide (24.28 g, 89.7 mmol, 2.30 equiv) dropwise with stirring at 0° C. The resulting solution was stirred for 1 h at Rt. To this was added 2,2-dimethyloxan-4-one (5.00 g, 39.01 mmol, 1.00 equiv) at RT. The resulting solution was allowed to react an additional 12 h at RT. The reaction was then quenched by the addition of 200 mL of water/ice. The pH value of the solution was adjusted to 5 with Na₂CO₃(s). The resulting solution was extracted with 3×50 mL of dichloromethane and the organic layers combined and concentrated. The residue was dissolved in 100 mL of EA. The resulting mixture was washed with 2×50 mL of brine. The mixture was dried over anhydrous sodium sulfate and concentrated. This resulted in 5.6 g (65.53%) of 4-bromo-6,6-dimethyl-2,5-dihydropyran-3-carbaldehyde as light yellow oil. ¹H NMR (300 MHz, DMSO-d₆, ppm): 6 9.84 (s, 1H), 4.23 (t, J=2.5 Hz, 2H), 2.73 (t, J=2.5 Hz, 2H), 1.20 (s, 6H).

### Synthesis of 4-(4-chlorophenyl)-6,6-dimethyl-2,5-dihydropyran-3-carbaldehyde: Into a 100-mL round-bottom flask purged and maintained with an inert atmosphere of nitrogen, was added 4-bromo-6,6-dimethyl-2,5-dihydropyran-3-carbaldehyde (5.40 g, 24.6 mmol, 1.00 equiv), DCE (50.00 mL), benzeneboronic acid, p-chloro-(4.63 g, 29.6 mmol, 1.2 equiv), Na₂CO₃ (5.27 g, 49.2 mmol, 2 equiv), Pd(PPh₃)₂Cl₂ (1.73 g, 2.46 mmol, 0.1 equiv), water (5 mL). The resulting solution was stirred for 12 h at 60° C. The reaction mixture was cooled and diluted with 50 ml water. The resulting solution was extracted with 3×50 mL of ethyl acetate and the organic layers combined. The resulting mixture was washed with 50 mL of brine. The mixture was dried over anhydrous sodium sulfate and concentrated. The residue was applied onto a silica gel column with ethyl acetate/petroleum ether (1:10-2:5). This resulted in 5.2 g (84.14%) of 4-(4-chlorophenyl)-6,6-dimethyl-2,5-dihydropyran-3-carbaldehyde as light yellow oil. LC-MS: (ES, m/z): M+1: 251

Synthesis of methyl 2-bromo-4-(4-[[4-(4-chlorophenyl)-6,6-dimethyl-2,5-dihydropyran-3-yl]methyl]piperazin-1-yl)benzoate: Into a 100-mL 3-necked round-bottom flask, was added 4-(4-chlorophenyl)-6,6-dimethyl-2,5-dihydropyran-3-carbaldehyde (2.00 g, 7.9 mmol, 1.00 equiv), DCE (20.00 mL), methyl 2-bromo-4-(piperazin-1-yl)benzoate (2.36 g, 7.9 mmol, 1 equiv), Ti(Oi-Pr)₄ (6.74 g, 23.7 mmol, 3 equiv). The resulting solution was stirred for 3 h at Rt. This was followed by the addition of NaBH(OAc)₃ (3.35 g, 15.8 mmol, 2 equiv) in several batches at Rt. The resulting solution was allowed to react for 12 at 60° C. The reaction was then quenched by the addition of 10 mL of water. The resulting mixture was concentrated. The residue was applied onto a silica gel column with ethyl acetate/petroleum ether (1:10-2:5). This resulted in 3 g (70.44%) of methyl 2-bromo-4-(4-[[4-(4-chlorophenyl)-6,6-dimethyl-2,5-dihydropyran-3-yl]methyl]piperazin-1-yl)benzoate as light yellow oil. LC-MS: (ES, m/z): M+1: 533

### Synthesis of 2-bromo-4-(4-[[4-(4-chlorophenyl)-6,6-dimethyl-2,5-dihydropyran-3-yl]methyl]piperazin-1-yl)benzoic acid: Into a 40-mL vial, was placed methyl 2-bromo-4-(4-[[4-(4-chlorophenyl)-6,6-dimethyl-2,5-dihydropyran-3-yl]methyl]piperazin-1-yl)benzoate (500.00 mg, 0.937 mmol, 1.00 equiv), MeOH (2.50 mL), THE (2.50 mL), H₂O (2.50 mL), NaOH (150.00 mg, 3.750 mmol, 4.00 equiv). The resulting solution was stirred for 3 h at 30 degrees C. The resulting solution was diluted with 50 mL of H₂O. The pH value of the solution was adjusted to 5 with HCl (0.5 mol/L). The resulting solution was extracted with 3×5 mL of ethyl acetate and the organic layers combined. The resulting mixture was washed with 1×5 mL of brine. The mixture was dried over anhydrous sodium sulfate and concentrated. This resulted in 460 mg (94.48%) of 2-bromo-4-(4-[[4-(4-chlorophenyl)-6,6-dimethyl-2,5-dihydropyran-3-yl]methyl]piperazin-1-yl)benzoic acid as white solid. LC-MS: (ES, m/z): M+1:519/521

Synthesis of 2-bromo-4-(4-[[4-(4-chlorophenyl)-6,6-dimethyl-2,5-dihydropyran-3-yl]methyl]piperazin-1-yl)-N-[3-nitro-4-[(oxan-4-ylmethyl)amino]benzenesulfonyl]benzamide: Into a 40-mL vial, was placed 2-bromo-4-(4-[[4-(4-chlorophenyl)-6,6-dimethyl-2,5-dihydropyran-3-yl]methyl]piperazin-1-yl)benzoic acid (300.00 mg, 0.577 mmol, 1.00 equiv), 4-[(4-methoxy-2-methylbutyl)amino]-3-nitrobenzenesulfonamide (182.00 mg, 0.573 mmol, 0.99 equiv), DCM (5.00 mL), DMAP (281.80 mg, 2.307 mmol, 4.00 equiv), EDCI (221.60 mg, 1.156 mmol, 2.00 equiv). The resulting solution was stirred for 12 h at 30 degrees C. The resulting mixture was concentrated. The residue was applied onto a silica gel column with dichloromethane/methanol (100:1-10:1). This resulted in 200 mg (42.41%) of 2-bromo-4-(4-[[4-(4-chlorophenyl)-6,6-dimethyl-2,5-dihydropyran-3-yl]methyl]piperazin-1-yl)-N-[3-nitro-4-[(oxan-4-ylmethyl)amino]benzenesulfonyl]benzamide as yellow solid. LC-MS: (ES, m/z): M+1: 816/818

### Synthesis of 4-(4-[[4-(4-chlorophenyl)-6,6-dimethyl-2,5-dihydropyran-3-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial purged and maintained with an inert atmosphere of nitrogen, was placed 2-bromo-4-(4-[[4-(4-chlorophenyl)-6,6-dimethyl-2,5-dihydropyran-3-yl]methyl]piperazin-1-yl)-N-[3-nitro-4-[(oxan-4-ylmethyl)amino]benzenesulfonyl]benzamide (250.00 mg, 0.306 mmol, 1.00 equiv), (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (97.80 mg, 0.306 mmol, 1.00 equiv), DMF (4.00 mL), CuI (11.60 mg, 0.061 mmol, 0.20 equiv), Cs₂CO₃ (199.50 mg, 0.612 mmol, 2.00 equiv), N1,N2-bis(4-hydroxy-2,6-dimethylphenyl)oxalamide (30.14 mg, 0.092 mmol, 0.30 equiv). The resulting solution was stirred for 2 h at 100 degrees C. The reaction mixture was cooled to room temperature. The resulting solution was diluted with 10 mL of H₂O. The resulting solution was extracted with 3×10 mL of ethyl acetate and the organic layers combined and dried over anhydrous sodium sulfate. The residue was applied onto a silica gel column with ethyl acetate/petroleum ether (3:1-0:1). This resulted in 120 mg (37.15%) of 4-(4-[[4-(4-chlorophenyl)-6,6-dimethyl-2,5-dihydropyran-3-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as yellow solid. LC-MS: (ES, m/z): M+1: 1055

Synthesis of 4-(4-[[4-(4-chlorophenyl)-6,6-dimethyl-2,5-dihydropyran-3-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial, was placed 4-(4-[[4-(4-chlorophenyl)-6,6-dimethyl-2,5-dihydropyran-3-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide (120.00 mg, 0.114 mmol, 1.00 equiv), DCM (3.00 mL), TFA (1.00 mL),The resulting solution was stirred for 4 h at room temperature.

The resulting mixture was concentrated. Then added CH₃CN (4.00 mL), ethylenediamine (34.50 mg, 0.568 mmol, 5.00 equiv). The resulting solution was allowed to react for an additional 6 h at 60 degrees C. The resulting mixture was concentrated. The residue was applied onto Pre-TLC with dichloromethane/methanol (95:5). The crude product (80 mg) was purified by Prep-HPLC with the following conditions (IntelFlash-1): Column, C18; mobile phase, CH₃CN:H₂O=65% 0.5% NH₃.H₂O; Detector, UV 254 nm. This resulted in 30 mg (28.52%) of 4-(4-[[4-(4-chlorophenyl)-6,6-dimethyl-2,5-dihydropyran-3-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as yellow solid. LC-MS: (ES, m/z): M+1: 925. ¹H-NMR (300 MHz, DMSO-d₆, ppm): δ12.12 (s, 1H), 10.99 (s, 1H), 8.52 (s, 1H), 8.36 (s, 1H), 7.56 (d, J=8.9 Hz, 1H), 7.39 (d, J=8.4 Hz, 2H), 7.15 (d, J=8.4 Hz, 2H), 7.03 (s, 1H), 6.78 (d, J=26.3 Hz, 2H), 6.53 (s, 1H), 5.97 (s, 1H), 4.15 (s, 2H), 3.93-3.78 (m, 2H), 3.31-3.03 (m, 10H), 2.87 (s, 2H), 2.21 (d, J=25.5 Hz, 6H), 1.89 (s, 1H), 1.63 (d, J=13.0 Hz, 2H), 1.50-1.25 (m, 4H), 1.20 (s, 6H).

### Compound 4-7: Preparation of 4-(4-[[2-(4-chlorophenyl)-5-methoxy-5-methylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide

Synthesis of (E)-2-(2-ethoxyvinyl)-4,4,5,5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil.LC-MS: (ES, m/z): M+1=199, R.T=1.007 min, 1.148 min. H-NMR-PH-PHNW-INT-A: (300 MHz, DMSO-d₆, ppm): δ6.91-6.95 (m, 1H), 4.30-4.34 (m, 1H), 3.78-3.82 (m, 2H), 1.14-1.24 (m, 15H).

Synthesis of N-[(2R)-2-hydroxypropyl]acetamide: Into a 5-mL 3-necked round-bottom flask purged and maintained with an inert atmosphere of nitrogen, was placed (2R)-1-aminopropan-2-ol (100 g, 1.3 mol, 1 equiv), DCM (1 L), TEA (160 g, 1.6 mol, 1.2 equiv). This was followed by the addition of a solution of acetyl acetate (136 g, 1.3 mol, 1.0 equiv) in 100 ml (DCM) dropwise with stirring at 0-10° C. The resulting solution was stirred for 14 hr at room temperature. The resulting mixture was concentrated. The residue was applied onto a silica gel column with dichloromethane/methanol (100:5). This resulted in 250 g (crude) of N-[(2R)-2-hydroxypropyl]acetamide as a yellow oil.

Synthesis of 5-bromo-6-fluoropyridin-2-amine: Into a 50L 4-necked round-bottom flask, was placed 6-fluoropyridin-2-amine (4500.00 g, 40.178 mol, 1.00 equiv), ACN (20.00 L). This was followed by the addition of NBS (7035.17 g, 41.383 mol, 1.03 equiv) in portions at R.T (25) degrees C. The resulting solution was stirred for 2 h at room temperature. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as a light brown solid.LC-MS: (ES, m/z): M+1=190, 193, R.T=0.926 min. H-NMR: (300 MHz, DMSO-d₆, ppm): δ7.63-7.71 (m, 1H), 6.56 (s, 2H), 6.30-631 (m, 1H).

Synthesis of 5-bromo-6-fluoro-3-iodopyridin-2-amine: Into a 50L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 2h at room temperature. The resulting solution was diluted with 100 L of water. The mixture was filtrated, collection of filter cake and the filter cake was washed by water (35L×2) and dried by oven to give product 9840 g(Y=90%) of 5-bromo-6-fluoro-3-iodopyridin-2-amine as a brown solid.LC-MS: (ES, m/z): M+1=317, 319, R.T=1.087 min. H-NMR: (300 MHz, DMSO-d₆, ppm): δ8.16-8.23 (m, 1H), 6.69 (s, 2H).

Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature in a liquid nitrogen bath. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil.LC-MS: (ES, m/z): M+1=261, 263, R.T=1.090 min. ¹H-NMR (300 MHz, DMSO-d₆, ppm): δ7.96-7.99 (m, 1H), 6.72-6.76 (m, 1H), 5.49-5.53 (m, 1H), 3.99-4.06 (m, 3H).

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as a light brown solid. LC-MS: (ES, m/z): M+1=215, 217, R.T=0.993 min. ¹H-NMR (300 MHz, DMSO-d₆, ppm): δ9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS: (ES, m/z): M+1=345, 347, R.T=1.435 min. ¹H-NMR (300 MHz, DMSO-d₆, ppm): δ8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

### Synthesis of (R)-N-(2-(5-bromo-1-((2-(trimethylsilyl)ethoxy)methyl)-1H-pyrrolo[2,3-b]pyidin-6-yloxy)propyl)acetamide: Into a 1L round-bottom flask, was placed N-[(2R)-2-hydroxypropyl] acetamide (crude, 81.6 g, 697 mmol, 3.00 equiv), dioxane (800 ml). This was followed by the addition of NaH (28 g, 697 mmol, 3 equiv), in portions at 5° C.-15° C. The resulting solution was stirred for 30 min at RT. To this was added a solution of 5-bromo-6-fluoro-1-((2-(trimethylsilyl)ethoxy)methyl)-1H-pyrrolo[2,3-b] pyridine (80 g, 232 mmol, 1 equiv) in dioxane (100 ml). The resulting solution was stirred for 4 hr at 80° C. in an oil bath. The reaction was then quenched by the addition of 50 ml of water at 10° C. The resulting solution was concentrated and diluted with 500 ml H₂O. The resulting solution was extracted with 3×500 ml of ethyl acetate. The resulting mixture was washed with 1×300 ml of NaCl (aq). The mixture was dried over anhydrous sodium sulfate and concentrated. The residue was applied onto a silica gel column with PE/EA (100:20). This resulted in 40 g (pure) and 30 g (70%) of (R)-N-(2-(5-bromo-1-((2-(trimethylsilyl)ethoxy)methyl)-1H-pyrrolo[2,3-b]pyridin-6-yloxy)propyl)acetamide as a yellow oil

Synthesis of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one: Into a 1L round-bottom flask purged and maintained with an inert atmosphere of nitrogen, was placed (R)-N-(2-(5-bromo-1-((2-(trimethylsilyl)ethoxy)methyl)-1H-pyrrolo[2,3-b]pyridin-6-yloxy)propyl)acetamide (40 g, 90.7 mmol, 1 equiv), dioxane (500 mL), Cs₂CO₃ (88 g, 272 mmol, 3 equiv), BrettPhos Pd G3 (4.1 g, 4.5 mmol, 0.05 equiv). The resulting solution was stirred for 6 hr at 80° C. in an oil bath. The resulting solution was concentrated and diluted with 300 ml H₂O. The resulting solution was extracted with 3×300 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (40:100). This resulted in 33 g(crude, 70%) of 1-[(12R)-4-(trimethylsilyl)ethoxy)methyl)-12-methyl-13-oxa-2,4,10-triazatricyclo[7.4.0.0\hat{ }[3,7]]trideca-1(9),2,5,7-tetraen-10-yl]ethan-1-one as brown oil.

Synthesis of (12R)-4-(trimethylsilyl)ethoxy)methyl)-12-methyl-13-oxa-2,4,10-triazatricyclo[7.4.0.0\hat{ }[3,7]]trideca-1(9),2,5,7-tetraene: Into a 1-L vial, was placed 1-[(12R)-4-(trimethylsilyl)ethoxy) methyl)-12-methyl-13-oxa-2,4,10-triazatricyclo[7.4.0.0\hat{ }[3,7]]trideca-1(9),2,5,7-tetraen-10-yl]ethan-1-one (33 g), methanol (300 mL), sodium hydroxide (2M, 300 ml). The resulting solution was stirred for 6 hr at 80° C. in an oil bath. The resulting solution was concentrated(MeOH) and extracted with 3×200 ml of ethyl acetate. The resulting mixture was washed with 1×200 mL of NaCl (aq). The mixture was dried over anhydrous sodium sulfate and concentrated. The residue was applied onto a silica gel column with ethyl acetate/petroleum ether (1:1). This resulted in 16.2 g of (12R)-4-(trimethylsilyl)ethoxy)methyl)-12-methyl-13-oxa-2,4,10-triazatricyclo [7.4.0.0\hat{ }[3,7]]trideca-1(9),2,5,7-tetraene as brown oil.

### Synthesis of methyl 2-bromo-4-(4-[[2-(4-chlorophenyl)-5-methoxy-5-methylcyclohex-1-en-1-yl]methyl]piperazin-1-yl)benzoate: Into a 100-mL 3-necked round-bottom flask, was added methyl 2-bromo-4-(piperazin-1-yl)benzoate (2.00 g, 6.68 mmol, 1.00 equiv), DCE (20 mL), 2-(4-chlorophenyl)-5-methoxy-5-methylcyclohex-1-ene-1-carbaldehyde (1.77 g, 6.685 mmol, 1 equiv), Ti(Oi-Pr)₄ (5.70 g, 20.05 mmol, 3.00 equiv). The resulting solution was stirred for 3 h at RT. This was followed by the addition of NaBH(OAc)₃ (2.83 g, 13.37 mmol, 2.00 equiv) in several batches at RT. The resulting solution was allowed to react, for an additional 16 h at RT. The reaction was then quenched by the addition of 10 mL of water. The resulting mixture was concentrated. The residue was applied onto a silica gel column with ethyl acetate/petroleum ether (1:10-2:5). This resulted in 3.0 g (81.90%) of methyl 2-bromo-4-(4-[[2-(4-chlorophenyl)-5-methoxy-5-methylcyclohex-1-en-1-yl]methyl]piperazin-1-yl)benzoate as light yellow oil.LC-MS (ES, m/z): 547 [M+H]⁺

Synthesis of 2-bromo-4-(4-[[2-(4-chlorophenyl)-5-methoxy-5-methylcyclohex-1-en-1-yl]methyl]piperazin-1-yl)benzoic acid: Into a 40-mL vial, was placed methyl 2-bromo-4-(4-[[2-(4-chlorophenyl)-5-methoxy-5-methylcyclohex-1-en-1-yl]methyl]piperazin-1-yl)benzoate (500.00 mg, 0.913 mmol, 1.00 equiv), MeOH (2.50 mL), THE (2.50 mL), H₂O (2.50 mL), NaOH (146.00 mg, 3.650 mmol, 4 equiv). The resulting solution was stirred for 3 hr at 30 degrees C. The resulting solution was diluted with 5 mL of H₂O. The pH value of the solution was adjusted to 5 with HCl (0.5 mol/L). The resulting solution was extracted with 3×5 mL of ethyl acetate. The resulting mixture was washed with 1×5 mL of brine. The mixture was dried over anhydrous sodium sulfate and concentrated. This resulted in 450 mg (92.37%) of 2-bromo-4-(4-[[2-(4-chlorophenyl)-5-methoxy-5-methylcyclohex-1-en-1-yl]methyl]piperazin-1-yl)benzoic acid as white solid. LC-MS (ES, m/z): M+1: 533/535

### Synthesis of 2-bromo-4-(4-[[2-(4-chlorophenyl)-5-methoxy-5-methylcyclohex-1-en-1-yl]methyl]piperazin-1-yl)-N-[3-nitro-4-[(oxan-4-ylmethyl)amino]benzenesulfonyl]benzamide: Into a 40-mL vial, was placed 2-bromo-4-(4-[[2-(4-chlorophenyl)-5-methoxy-5-methylcyclohex-1-en-1-yl]methyl]piperazin-1-yl)benzoic acid (490.00 mg, 0.918 mmol, 1.00 equiv), 3-nitro-4-[(oxan-4-ylmethyl)amino]benzenesulfonamide (289.50 mg, 0.918 mmol, 1.00 equiv), DCM (8.00 mL), DMAP (448.50 mg, 3.671 mmol, 4 equiv), EDCI (352.50 mg, 1.839 mmol, 2.00 equiv). The resulting solution was stirred for 12 hr at 30 degrees C. The resulting mixture was concentrated. The residue was applied onto a silica gel column with dichloromethane/methanol (10:1). This resulted in 500 mg (65.54%) of 2-bromo-4-(4-[[2-(4-chlorophenyl)-5-methoxy-5-methylcyclohex-1-en-1-yl]methyl]piperazin-1-yl)-N-[3-nitro-4-[(oxan-4-ylmethyl)amino]benzenesulfonyl]benzamide as yellow solid. LC-MS (ES, m/z): M+1: 830/832

Synthesis of 4-(4-[[2-(4-chlorophenyl)-5-methoxy-5-methylcyclohex-1-en-1-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial purged and maintained with an inert atmosphere of nitrogen, was placed 2-bromo-4-(4-[[2-(4-chlorophenyl)-5-methoxy-5-methylcyclohex-1-en-1-yl]methyl]piperazin-1-yl)-N-[3-nitro-4-[(oxan-4-ylmethyl)amino]benzenesulfonyl]benzamide (400.00 mg, 0.481 mmol, 1.00 equiv), (12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraene (153.70 mg, 0.481 mmol, 1.00 equiv), CuI (18.33 mg, 0.096 mmol, 0.2 equiv), Cs₂CO₃ (313.60 mg, 962.495 mmol, 2000.11 equiv), 4-hydroxy-2,6-dimethylphenyl [(4-hydroxy-2,6-dimethylphenyl)carbamoyl]formate (47.40 mg, 0.144 mmol, 0.30 equiv), DMF (6.00 mL, 0.082 mmol, 0.17 equiv). The resulting solution was stirred for 2 hr at 100 degrees C. The reaction mixture was cooled to room temperature. The resulting solution was diluted with 10 mL of H₂O. The resulting solution was extracted with 3×10 mL of ethyl acetate and the organic layers combined and dried over anhydrous sodium sulfate and concentrated. The residue was applied onto a silica gel column with ethyl acetate/petroleum ether (1:3-0:1). This resulted in 250 mg (48.56%) of 4-(4-[[2-(4-chlorophenyl)-5-methoxy-5-methylcyclohex-1-en-1-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as a yellow solid. LC-MS (ES, m/z): M+1: 1069

Synthesis of 4-(4-[[2-(4-chlorophenyl)-5-methoxy-5-methylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide: Into a 40-mL vial, was placed 4-(4-[[2-(4-chlorophenyl)-5-methoxy-5-methylcyclohex-1-en-1-yl]methyl]piperazin-1-yl)-2-[(12R)-12-methyl-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide (200.00 mg, 0.187 mmol, 1.00 equiv), DCM (3.00 mL), TFA (1.00 mL), The resulting solution was stirred for 4 hr at Rt degrees C. The resulting mixture was concentrated. Then added CH₃CN (4.00 mL), ethane-1,2-diamine (56.70 mg, 0.943 mmol, 5.05 equiv). The resulting solution was allowed to react for 6 hr at 60 degrees C. The resulting mixture was concentrated. The residue was applied onto Pre-TLC with dichloromethane/methanol (95:5). The crude product (130 mg) was purified by Prep-HPLC with the following conditions (IntelFlash-1): Column, C18; mobile phase, CH₃CN:H₂O=65% 0.5% NH₃.H₂O; Detector, UV 254 nm. This resulted in 50 mg (28.47%) of 4-(4-[[2-(4-chlorophenyl)-5-methoxy-5-methylcyclohex-1-en-1-yl]methyl]piperazin-1-yl)-2-[(12R)-12-methyl-13-oxa-2,4,10-triazatricyclo[7.4.0.0\hat{ }[3,7]]trideca-1(9),2,5,7-tetraen-10-yl]-N-[3-nitro-4-[(oxan-4-ylmethyl)amino]benzenesulfonyl]benzamide as yellow solid.LC-MS (ES, m/z): M+1:939. ¹H-NMR (300 MHz, DMSO-d₆, ppm) δ12.11 (s, 1H), 10.99 (s, 1H), 8.52 (s, 1H), 8.37 (s, 1H), 7.56 (d, J=8.7 Hz, 1H), 7.42-7.32 (m, 2H), 7.15-7.06 (m, 2H), 7.03 (s, 1H), 6.81 (s, 1H), 6.73 (s, 1H), 6.64-6.41 (m, 2H)5.97 (s, 1H), 4.54 (s, 2H), 3.96-3.76 (m, 2H), 3.30 (s, 6H), 3.16-3.10 (m, 4H), 2.74 (s, 2H), 2.42-2.05 (m, 8H), 1.90 (d, J=9.1 Hz, 1H), 1.83-1.71 (m, 1H), 1.63 (d, J=12.5 Hz, 3H), 1.28 (tt, J=12.1, 6.1 Hz, 5H), 1.17 (s, 3H).

### Compound 4-8: Preparation of 4-(4-[[2-(4-chlorophenyl)-4, 4-dimethylcyclohex-1-en-1-yl] methyl] piperazin-1-yl)-N-[3-nitro-4-[(oxan-4-ylmethyl) amino] benzenesulfonyl]-2-[(12R)-12-(trifluoromethyl)-13-oxa-2,4,10-triazatricyclo[7.4.0.0\hat{ }[3,7]]trideca-1(9),2,5,7-tetraen-10-yl]benzamide(Assumed

Synthesis of 5-bromo-6-fluoropyridin-2-amine: Into a 50L 4-necked round-bottom flask, was placed 6-fluoropyridin-2-amine (4500.00 g, 40.178 mol, 1.00 equiv), ACN (20.00 L). This was followed by the addition of NBS (7035.17 g, 41.383 mol, 1.03 equiv) in portions at R.T (25) degrees C. The resulting solution was stirred for 3 h at room temperature. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as a light brown solid. LC-MS: M+1=190, 193. H-NMR: 6 7.63-7.71 (m, 1H), 6.56 (s, 2H), 6.30-631 (m, 1H).

Synthesis of 5-bromo-6-fluoro-3-iodopyridin-2-amine: Into a 50L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 3h at room temperature. The resulting solution was diluted with 100 L of water. The mixture was filtrated, collection of filter cake and the filter cake was washed by water (35L×2) and dried by oven to give product 9840 g(Y=90%) of 5-bromo-6-fluoro-3-iodopyridin-2-amine as a brown solid. LC-MS: M+1=317, 319. H-NMR: 6 8.16-8.23 (m, 1H), 6.69 (s, 2H).

Synthesis of (E)-2-(2-ethoxyvinyl)-4, 4, 5, 5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil. LC-MS: M+1=199. H-NMR: 6 6.91-6.95 (m, 1H), 4.30-4.34 (m, 1H), 3.78-3.82 (m, 2H), 1.14-1.24 (m, 15H).

Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil. LC-MS: M+1=261, 263. H-NMR: 6 7.96-7.99 (m, 1H), 6.72-6.76 (m, 1H), 5.49-5.53 (m, 1H), 3.99-4.06 (m, 3H).

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as a light brown solid. LC-MS: M+1=215, 217. H-NMR: 6 9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS: M+1=345, 347. H-NMR: 6 8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

Synthesis of methyl 2-bromo-4-(4-((2-(4-chlorophenyl)-4, 4-dimethylcyclohex-1-enyl) methyl) piperazin-1-yl) benzoate: Into a 20000-mL round-bottom flask, was placed 1-((2-(4-chlorophenyl)-4,4-dimethylcyclohex-1-enyl)methyl)piperazine dihydrochloride (600 g, 1.53 mol, 1 equiv), methyl 2-bromo-4-fluorobenzoate (357 g, 1.53 mol, 1 equiv), DBU (319 g, 6.12 mol, 4 equiv) and DMSO (8000 mL). The resulting solution was stirred for 20 h at 70 degrees C. LCMS showed material was completely consumed. The resulting mixture was cooled to R.T and poured into water (32 L). The mixture was filtrated, collection of filter cake and the filter cake was washed by water (3000 mL×3) and dried by oven to give product 740 g (Y: 91%) methyl 2-bromo-4-(4-((2-(4-chlorophenyl)-4,4-dimethylcyclohex-1-enyl)methyl)piperazin-1-yl)benzoate as a white solid. LC-MS: M+1=531. H-NMR: 6 7.73 (d, J=9.0 Hz, 1H), 7.42-7.39 (m, 2H), 7.18-7.12 (m, 3H), 6.97-6.94 (m, 1H), 4.00-3.84 (m, 2H), 3.76 (s, 2H), 3.57 (s, 3H), 3.51-3.33 (m, 4H), 2.79-2.60 (m, 2H), 2.32-2.30 (m, 2H), 2.03-1.97 (m, 2H), 1.47-1.45 (m, 2H), 0.96 (s, 6H).

Synthesis of 2-bromo-4-(4-((2-(4-chlorophenyl)-4, 4-dimethylcyclohex-1-enyl) methyl) piperazin-1-yl) benzoic acid: Into a 20000-mL round-bottom flask, was placed methyl 2-bromo-4-(4-((2-(4-chlorophenyl)-4, 4-dimethylcyclohex-1-enyl) methyl) piperazin-1-yl) benzoate (730 g, 1.37 mol, 1 equiv), LiOH (131.5 g, 5.48 mol, 4 equiv) and MeOH/THF/water (4500 mL/3000 mL/1000 mL). The resulting solution was stirred for 16 h at 70 degrees C. LCMS showed material was completely consumed. The resulting mixture was cooled to R.T and concentrated. The residue was diluted with water (5000 mL) and the mixture was adjust PH to 3-5 with HCl (6 M), followed by filtrated, collection of filter cake and dried by oven to give product 650 g (Y=93%) 2-bromo-4-(4-((2-(4-chlorophenyl)-4, 4-dimethylcyclohex-1-enyl) methyl) piperazin-1-yl) benzoic acid as a white solid. LC-MS: M+1=517. H-NMR: 6: 10.60 (bs, 1H), 7.73 (d, J=8.4 Hz, 1H), 7.42-7.39 (m, 2H), 7.14-7.11 (m, 3H), 6.95-6.92 (m, 1H), 4.00-3.84 (m, 2H), 3.76 (s, 2H), 3.51-3.33 (m, 4H), 2.79-2.60 (m, 2H), 2.32-2.30 (m, 2H), 2.03-1.97 (m, 2H), 1.47-1.45 (m, 2H), 0.97 (s, 6H).

Synthesis of 2-bromo-4-(4-((2-(4-chlorophenyl)-4, 4-dimethylcyclohex-1-enyl) methyl) piperazin-1-yl)-N-(3-nitro-4-((tetrahydro-2H-pyran-4-yl) methylamino) phenylsulfonyl) benzamide: Into a 20000-mL round-bottom flask, was placed 2-bromo-4-(4-((2-(4-chlorophenyl)-4, 4-dimethylcyclohex-1-enyl)methyl)piperazin-1-yl)benzoic acid (550 g, 1.07 mol, 1 equiv), DCM (10 L), 3-nitro-4-[[(oxan-4-yl)methyl]amino]benzene-1-sulfonamide (320 g, 1.02 mol, 0.95 equiv), EDCI (308 g, 1.61 mol, 1.5 equiv), DMAP (522 g, 4.28 mol, 4 equiv). The resulting solution was stirred for overnight at 25 degrees C. LCMS showed material was completely consumed. The resulting mixture is followed by dilute hydrochloric acid (1.0 M) (1000 mL×3), saturated sodium bicarbonate (1000 mL×3) and brine (1000 mL×1), and then the organic phase was dried by Na₂SO₄, filtrated. The filtrate was concentrated to give product 810 g (Y: 93%) as a light brown yellow solid 2-bromo-4-(4-((2-(4-chlorophenyl)-4, 4-dimethylcyclohex-1-enyl) methyl) piperazin-1-yl)-N-(3-nitro-4-((tetrahydro-2H-pyran-4-yl) methylamino) phenylsulfonyl) benzamide as a brown yellow solid. LC-MS: M+1=814. H-NMR: 6 8.63-8.61 (m, 2H), 7.94-7.92 (m, 1H), 7.37-7.35 (m, 3H), 7.27-7.24 (m, 1H), 7.05-7.02 (m, 3H), 6.86-6.83 (m, 1H), 3.87-3.82 (m, 2H), 3.37-3.23 (m, 8H), 2.92 (s, 2H), 2.50-2.38 (m, 4H), 2.22-2.20 (m, 2H), 2.00-1.97 (m, 2H), 1.64-1.60 (m, 2H), 1.48-1.46 (m, 2H), 1.26-1.20 (m, 2H), 0.97 (s, 6H).

Synthesis of 4-methyl-N-(3, 3, 3-trifluoro-2-hydroxypropyl)benzenesulfonamide: Into a 100-mL 3-necked round-bottom flask, was placed 3-amino-1, 1, 1-trifluoropropan-2-ol (950.00 mg, 7.364 mmol, 1.00 equiv), TEA (1.49 g, 14.729 mmol, 2.00 equiv), DCM (25.00 mL). This was followed by the addition of P-toluenesulfonyl chloride (1.40 g, 7.364 mmol, 1.00 equiv) dropwise with stirring at 0 degrees C. The resulting solution was stirred for 3h at room temperature in a water bath. The reaction was then quenched by the addition of 50 mL of water. The resulting solution was extracted with 3×50 mL of dichloromethane and the organic layers combined. The resulting mixture was washed with 2×50 ml of water and 1×50 mL of brine. The mixture was dried over anhydrous sodium sulfate and concentrated. The residue was applied onto a silica gel column with ethyl acetate/petroleum ether (1:1). This resulted in 2 g of 4-methyl-N-(3, 3, 3-trifluoro-2-hydroxypropyl) benzenesulfonamide as colorless oil. LC-MS: M+1=284.

Synthesis of N-[2-[(5-bromo-1-[[2-(trimethylsilyl) ethoxy] methyl] pyrrolo [2, 3-b] pyridin-6-yl) oxy]-3, 3, 3-trifluoropropyl]-4-methylbenzenesulfonamide: Into a 250-mL round-bottom flask, was placed 4-methyl-N-(3, 3, 3-trifluoro-2-hydroxypropyl) benzenesulfonamide (2.00 g, 7.067 mmol, 1.00 equiv), 5-bromo-6-fluoro-1-[[2-(trimethylsilyl) ethoxy] methyl] pyrrolo[2,3-b] pyridine (2.40 g, 7.067 mmol, 1.00 equiv), Cs₂CO₃ (4.60 g, 14.134 mmol, 2.00 equiv), Dioxane (50.00 mL). The resulting solution was stirred for 16h at 90 degrees C. in an oil bath. The reaction mixture was cooled to room temperature with a water bath. The reaction was then quenched by the addition of 50 mL of water. The solution was filtrated and filter cake was washed by EA (50 ml) three times. The resulting mixture was concentrated. The residue was applied onto a silica gel column with ethyl acetate/petroleum ether (1:3). This resulted in 2.7 g of N-[2-[(5-bromo-1-[[2-(trimethylsilyl) ethoxy] methyl] pyrrolo[2, 3-b] pyridin-6-yl) oxy]-3, 3, 3-trifluoropropyl]-4-methylbenzenesulfonamide as colorless oil. LC-MS: M+1=608.

Synthesis of 10-(4-methylbenzenesulfonyl)-12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene: Into a 100-mL round-bottom flask purged and maintained with an inert atmosphere of nitrogen, was placed N-[2-[(5-bromo-1-[[2-(trimethylsilyl)ethoxy]methyl]pyrrolo[2, 3-b]pyridin-6-yl) oxy]-3, 3, 3-trifluoropropyl]-4-methylbenzenesulfonamide (2.70 g, 4.448 mmol, 1.00 equiv), 2-(2-methylpropanoyl) cyclohexan-1-one (0.37 g, 2.224 mmol, 0.50 equiv), CuI (0.84 g, 4.448 mmol, 1.00 equiv), DMSO (50.00 mL), Cs₂CO₃ (4.30 g, 13.344 mmol, 3.00 equiv). The resulting solution was stirred for 2h at 120 degrees C. in an oil bath. The reaction mixture was cooled to room temperature with a water bath. The resulting solution was diluted with 200 mL of water. The resulting solution was extracted with 3×150 mL of ethyl acetate and the organic layers combined. The resulting mixture was washed with 3×100 ml of water and 1×100 mL of brine. The mixture was dried over anhydrous sodium sulfate and concentrated. The residue was applied onto a silica gel column with ethyl acetate/petroleum ether (1:3). This resulted in 2.3 g of 10-(4-methylbenzenesulfonyl)-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraene as light yellow oil. LC-MS: M+1=528.

Synthesis of 12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene: Into a 50-mL round-bottom flask purged and maintained with an inert atmosphere of nitrogen, was placed 10-(4-methylbenzenesulfonyl)-12-(trifluoromethyl)-4-[[2-(trimethylsilyl) ethoxy] methyl]-13-oxa-2, 4, 10-triazatricyclo[7.4.0.0\hat{ }[3,7]] trideca-1(9),2,5,7-tetraene (1.30 g, 2.467 mmol, 1.00 equiv), MeOH (10.00 mL), Mg (200.00 mg, 4.934 mmol, 2.00 equiv). The resulting solution was stirred for 18h at room temperature. The solids were filtered out. The resulting mixture was concentrated. The residue was applied onto a silica gel column with ethyl acetate/petroleum ether (1:3). This resulted in 700 mg of 12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene as colorless oil. LC-MS: M+1=374. ¹H-NMR: 6 7.31-7.32 (d, J=3.0 Hz, 1H), 7.25 (s, 1H), 6.28-6.28 (d, J=3.0 Hz, 1H), 5.83 (s, 1H), 5.43 (s, 2H), 5.05-5.06 (d, J=3.0 Hz, 1H), 3.56-3.41 (m, 3H), 3.31-3.18 (m, 1H), 0.92-0.73 (m, 3H),-0.09 (s, 9H).

Synthesis of (12R)-12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene(Assumed): The 300 mg 12-(trifluoromethyl)-4-[[2-(trimethylsilyl) ethoxy] methyl]-13-oxa-2, 4, 10-triazatricyclo [7.4.0.0\hat{ }[3, 7]]trideca-1(9),2,5,7-tetraene was purified by Chiral-Prep-HPLC with the following conditions: Column, CHIRALPAK OJ-3, 50*4.6 mm, 3 um, OJ30CC-QK005; mobile phase A: n-Hexane, Mobile Phase B: Ethanol; Flow rate: 1.0 mL/min; Gradient: 0% B to 5% B in 6 min; Detector, 220 nm. This resulted in 130 mg (98%) of (12Rassumed)-12-(trifluoromethyl)-4-[[2-(trimethylsilyl) ethoxy] methyl]-13-oxa-2,4,10-triazatricyclo [7.4.0.0\hat{ }[3,7]]trideca-1(9),2,5,7-tetraene(Assumed). LC-MS: M+1=374. ee=98.78%.

Synthesis of 4-(4-[[2-(4-chlorophenyl)-4, 4-dimethylcyclohex-1-en-1-yl] methyl]piperazin-1-yl)-N-[3-nitro-4-[(oxan-4-ylmethyl)amino]benzenesulfonyl]-2-[(12R)-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide(Assumed): Into a 50-mL round-bottom flask purged and maintained with an inert atmosphere of nitrogen, was placed 2-bromo-4-(4-[[2-(4-chlorophenyl)-4, 4-dimethylcyclohex-1-en-1-yl] methyl]piperazin-1-yl)-N-[3-nitro-4-[(oxan-4-ylmethyl)amino]benzenesulfonyl]benzamide (140.00 mg, 0.172 mmol, 1.30 equiv), (12R)-12-(trifluoromethyl)-4-[[2-(trimethylsilyl) ethoxy]methyl]-13-oxa-2, 4, 10-triazatricyclo [7.4.0.0\hat{ }[3,7]] trideca-1(9),2,5,7-tetraene (Assumed) (50.00 mg, 0.133 mmol, 1.00 equiv), N₁,N₂-bis(4-hydroxy-2,6-dimethylphenyl)oxalamide (10.00 mg, 0.026 mmol, 0.20 equiv), Cs₂CO₃ (110.00 mg, 0.331 mmol, 2.50 equiv), CuI (10.00 mg, 0.053 mmol, 0.40 equiv), DMF (10.00 mL). The resulting solution was stirred for 2h at 100 degrees C. in an oil bath. The reaction mixture was cooled to room temperature with a water bath. The resulting solution was diluted with 60 ml of EA. The solids were filtered out. The mixture was dried over anhydrous sodium sulfate and concentrated. The residue was applied onto a silica gel column with dichloromethane/methanol (10:1). This resulted in 120 mg of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[(12Rassumed)-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide(Assumed) as a yellow solid. LC-MS: M+1=1107.

Synthesis of 4-(4-[[2-(4-chlorophenyl)-4, 4-dimethylcyclohex-1-en-1-yl] methyl]piperazin-1-yl)-N-[3-nitro-4-[(oxan-4-ylmethyl)amino]benzenesulfonyl]-2-[(12R)-12-(trifluoromethyl)-13-oxa-2,4,10-triazatricyclo[7.4.0.0\hat{ }[3,7]]trideca-1(9),2,5,7-tetraen-10-yl]benzamide(Assumed): Into a 50-mL round-bottom flask, was placed 4-(4-[[2-(4-chlorophenyl)-4, 4-dimethylcyclohex-1-en-1-yl] methyl] piperazin-1-yl)-N-[3-nitro-4-[(oxan-4-ylmethyl) amino] benzenesulfonyl]-2-[(12R_{d})-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide(Assumed) (120.00 mg, 0.108 mmol, 1.00 equiv), ethylenediamine (1.00 mL), THE (5.00 mL), TBAF.3H₂O (43.00 mg, 0.163 mmol, 1.50 equiv). The resulting solution was stirred for 72h at 80 degrees C. in an oil bath. The reaction mixture was cooled to room temperature with a water bath. The resulting solution was diluted with 10 mL of water. The resulting solution was extracted with 3×10 mL of ethyl acetate and the organic layers combined. The resulting mixture was washed with 1×10 ml of brine. The mixture was dried over anhydrous sodium sulfate and concentrated. The residue was applied onto a silica gel column with dichloromethane/methanol (5:1). The crude product was purified by Prep-HPLC with the following conditions: column, X-Bridge Prep C18 19*150 mm Sum; mobile phase, A: water (it contains 10 mM NH₄HCO₃ 0.05% ammonia); B: ACN; Gradient: 20-45% B in 8 min; Flow rate: 20 mL/min; detector, UV 220 nm. The collected solution was concentrated under vacuum to remove CH₃CN and the resulting solution was dried by lyophilization. This resulted in 11 mg of 4-(4-[[2-(4-chlorophenyl)-4, 4-dimethylcyclohex-1-en-1-yl] methyl]piperazin-1-yl)-N-[3-nitro-4-[(oxan-4-ylmethyl) amino]benzenesulfonyl]-2-[(12R)-12-(trifluoromethyl)-13-oxa-2,4,10-triazatricyclo[7.4.0.0\hat{ }[3,7]]trideca-1(9),2,5,7-tetraen-10-yl]benzamide(Assumed) as a yellow solid. LC-MS: M+1=977. ¹H-NMR: 6 11.07 (s, 1H), 8.52 (s, 1H), 8.35 (s, 1H), 7.49 (s, 1H), 7.39 (s, 2H), 7.26-7.28 (d, J=6.0 Hz, 1H), 7.09 (s, 1H), 6.81-6.85 (d, J=12.0 Hz, 2H), 6.65 (s, 2H), 5.97 (s, 1H), 5.10 (s, 1H), 3.87-3.89 (d, J=6.0 Hz, 4H), 3.65-3.67 (m, 2H), 3.26-3.35 (m, 6H), 2.73-2.81 (m, 2H), 2.23-2.28 (m, 3H), 1.98-2.04 (m, 3H), 1.90 (s, 2H), 1.62-1.67 (m, 2H), 1.40-1.50 (m, 2H), 1.24-1.30 (m, 6H), 0.96 (s, 6H).

### Compound 4-9: Preparation of 4-(4-[[2-(4-chlorophenyl)-4, 4-dimethylcyclohex-1-en-1-yl] methyl] piperazin-1-yl)-N-[3-nitro-4-[(oxan-4-ylmethyl) amino] benzenesulfonyl]-2-[(12Sassumed)-12-(trifluoromethyl)-13-oxa-2,4,10-triazatricyclo[7.4.0.0\hat{ }[3,7]]trideca-1(9),2,5,7-tetraen-10-yl]benzamide

Synthesis of 5-bromo-6-fluoropyridin-2-amine: Into a 50L 4-necked round-bottom flask, was placed 6-fluoropyridin-2-amine (4500.00 g, 40.178 mol, 1.00 equiv), ACN (20.00 L). This was followed by the addition of NBS (7035.17 g, 41.383 mol, 1.03 equiv) in portions at R.T (25) degrees C. The resulting solution was stirred for 3 h at room temperature. The resulting solution was diluted with 40 L of water. The resulting solution was extracted with 2×36 L of ethyl acetate. The resulting mixture was washed with 10 L of brine. The mixture was dried over anhydrous sodium sulfate and concentrated. The resulting mixture was washed with 3×9L of PE. This mixture was dried by oven to give product 7634 g(Y=90%) of 5-bromo-6-fluoropyridin-2-amine as a light brown solid. LC-MS: M+1=190, 193. H-NMR: 6 7.63-7.71 (m, 1H), 6.56 (s, 2H), 6.30-631 (m, 1H).

Synthesis of 5-bromo-6-fluoro-3-iodopyridin-2-amine: Into a 50L 4-necked round-bottom flask, was placed 5-bromo-6-fluoropyridin-2-amine (7000.00 g, 36.842 mol, 1.00 equiv), AcOH (34.00 L). The solution was cooled to 10 degree C. in a water/ice bath. This was followed by the addition of NIS (8290 g, 36.8 mol, 1.00 equiv) in portions at 10 degrees C. The resulting solution was stirred for 3h at room temperature. The resulting solution was diluted with 100 L of water. The mixture was filtrated, collection of filter cake and the filter cake was washed by water (35L×2) and dried by oven to give product 9840 g(Y=90%) of 5-bromo-6-fluoro-3-iodopyridin-2-amine as a brown solid. LC-MS: M+1=317, 319. H-NMR: 6 8.16-8.23 (m, 1H), 6.69 (s, 2H).

Synthesis of (E)-2-(2-ethoxyvinyl)-4, 4, 5, 5-tetramethyl-1,3,2-dioxaborolane: Into a 3L 4-necked round-bottom flask, was placed ethyl vinyl ether (1000.00 g, 13.9 mol, 3.50 equiv), 4,4,5,5-tetramethyl-1,3,2-dioxaborolane (508.00 g, 3.97 mol, 1.00 equiv). This solution was cooled to 5 degrees C. in an ice/salt bath. This followed by the addition of Pd(OAc)₂ (50.00 g, 222 mmol, 0.02 equiv) in portions at 5 degrees C. The resulting solution was stirred for 18h at room temperature. The resulting solution was diluted with 5 L of PE. The resulting mixture was concentrated. This resulted in 1100 g (crude, Y=50%) of 2-[(E)-2-ethoxyethenyl]-4,4,5,5-tetramethyl-1,3-dioxolane as light yellow oil. LC-MS: M+1=199. H-NMR: 6 6.91-6.95 (m, 1H), 4.30-4.34 (m, 1H), 3.78-3.82 (m, 2H), 1.14-1.24 (m, 15H).

Synthesis of 5-bromo-3-(2-ethoxyvinyl)-6-fluoropyridin-2-amine: Into a 20L 4-necked round-bottom flask purged and maintained with an inert atmosphere of nitrogen, was placed 5-bromo-6-fluoro-3-iodopyridin-2-amine (855.00 g, 2705.696 mmol, 1.00 equiv), i-PrOH (10000.00 mL), 2-[(E)-2-ethoxyethenyl]-4,4,5,5-tetramethyl-1,3-dioxolane (1000.00 g, 5050.505 mmol, 1.87 equiv), K₃PO₄ (1720.00 g, 8113.207 mmol, 3.00 equiv), Ruphos (12.00 g, 27.060 mmol, 0.02 equiv), Pd(OAc)₂ (20.00 g, 88 mmol, 0.02 equiv). The resulting solution was stirred for 12h at room temperature. The solids were filtered out. The filter cake was washed by 3×2L DCM. The organic layer was collected and concentrated. The residue was applied onto a silica gel column and eluted with ethyl acetate/petroleum ether (1:5). This concentrated and resulted in 680 g (80%) of 5-bromo-3-[(E,Z)-2-ethoxyethenyl]-6-fluoropyridin-2-amine(Z,E mixtures) as dark brown oil. LC-MS: M+1=261, 263. H-NMR: 6 7.96-7.99 (m, 1H), 6.72-6.76 (m, 1H), 5.49-5.53 (m, 1H), 3.99-4.06 (m, 3H).

Synthesis of 5-bromo-6-fluoro-1H-pyrrolo [2, 3-b] pyridine: Into a 10 L 4-necked round-bottom flask purged and maintained with an inert atmosphere of nitrogen, was placed 5-bromo-3-[(E)-2-ethoxyethenyl]-6-fluoropyridin-2-amine (680.00 g, 2605.364 mmol, 1.00 equiv), EtOH (5000.00 mL), HCl (1000.00 mL). The resulting solution was stirred for 5h at room temperature. The resulting mixture was concentrated. The pH value of the solution was adjusted to 6 with NaOH (4 mol/L). The solids were collected by filtration and washed with 3×500 mL water. This resulted in 560 g (Q-NMR=70%) of 5-bromo-6-fluoro-1H-pyrrolo [2, 3-b] pyridine as a light brown solid. LC-MS: M+1=215, 217. H-NMR: 6 9.53 (brs, 1H), 8.19-8.22 (d, J=9.0 Hz, 1H), 7.32-7.34 (m, 1H), 6.50-6.52 (m, 1H).

Synthesis of 5-bromo-6-fluoro-1-((2-(trimethylsilyl)ethoxy)methyl)-1H-pyrrolo[2,3-b]pyridine: Into a 10 L 4-necked round-bottom flask was placed 5-bromo-6-fluoro-1H-pyrrolo [2, 3-b] pyridine (560.00 g, 2.6 mol, 1.00 equiv), DMF (5000.00 mL). This solution was cooled to 0 degrees C. in a water/ice bath. This was followed by the addition of NaH (156 g, 3.9 mol, 1.5 equiv) in portions at 0 degrees C. To this was added SEM-Cl (561 g, 3.38 mol, 1.3 equiv) dropwise with stirring at 0 degrees C. The resulting solution was stirred for 1 h at room temperature. The reaction was then quenched by the addition of 2000 mL of water/ice. The resulting solution was diluted with 5 L of water. The resulting solution was extracted with 2×10 L of ethyl acetate and the organic layers combined. The resulting mixture was washed with 3×5L of water. The resulting mixture was washed with 3 L of brine. The mixture was dried over anhydrous sodium sulfate and concentrated. The residue was applied onto a silica gel column and eluted with ethyl acetate/petroleum ether (1:20). This resulted in 550 g (87%) of 5-bromo-6-fluoro-1-[[2-(trimethylsilyl) ethoxy] methyl] pyrrolo [2, 3-b]pyridine as light yellow oil. LC-MS: M+1=345, 347. H-NMR: 6 8.46-8.49 (d, J=9.0 Hz, 1H), 7.68-7.69 (m, 1H), 6.57-6.58 (m, 1H), 5.52-5.55 (m, 2H), 3.47-3.60 (m, 2H), 0.79-0.90 (m, 2H), 0.01 (s, 9H).

Synthesis of methyl 2-bromo-4-(4-((2-(4-chlorophenyl)-4, 4-dimethylcyclohex-1-enyl) methyl) piperazin-1-yl) benzoate: Into a 20000-mL round-bottom flask, was placed 1-((2-(4-chlorophenyl)-4,4-dimethylcyclohex-1-enyl)methyl)piperazine dihydrochloride (600 g, 1.53 mol, 1 equiv), methyl 2-bromo-4-fluorobenzoate (357 g, 1.53 mol, 1 equiv), DBU (319 g, 6.12 mol, 4 equiv) and DMSO (8000 mL). The resulting solution was stirred for 20 h at 70 degrees C. LCMS showed material was completely consumed. The resulting mixture was cooled to R.T and poured into water (32 L). The mixture was filtrated, collection of filter cake and the filter cake was washed by water (3000 mL×3) and dried by oven to give product 740 g (Y: 91%) methyl 2-bromo-4-(4-((2-(4-chlorophenyl)-4,4-dimethylcyclohex-1-enyl)methyl)piperazin-1-yl)benzoate as a white solid. LC-MS: M+1=531. H-NMR: 6 7.73 (d, J=9.0 Hz, 1H), 7.42-7.39 (m, 2H), 7.18-7.12 (m, 3H), 6.97-6.94 (m, 1H), 4.00-3.84 (m, 2H), 3.76 (s, 2H), 3.57 (s, 3H), 3.51-3.33 (m, 4H), 2.79-2.60 (m, 2H), 2.32-2.30 (m, 2H), 2.03-1.97 (m, 2H), 1.47-1.45 (m, 2H), 0.96 (s, 6H).

Synthesis of 2-bromo-4-(4-((2-(4-chlorophenyl)-4, 4-dimethylcyclohex-1-enyl) methyl) piperazin-1-yl) benzoic acid: Into a 20000-mL round-bottom flask, was placed methyl 2-bromo-4-(4-((2-(4-chlorophenyl)-4, 4-dimethylcyclohex-1-enyl) methyl) piperazin-1-yl) benzoate (730 g, 1.37 mol, 1 equiv), LiOH (131.5 g, 5.48 mol, 4 equiv) and MeOH/THF/water (4500 mL/3000 mL/1000 mL). The resulting solution was stirred for 16 h at 70 degrees C. LCMS showed material was completely consumed. The resulting mixture was cooled to R.T and concentrated. The residue was diluted with water (5000 mL) and the mixture was adjust PH to 3-5 with HCl (6 M), followed by filtrated, collection of filter cake and dried by oven to give product 650 g (Y=93%) 2-bromo-4-(4-((2-(4-chlorophenyl)-4, 4-dimethylcyclohex-1-enyl) methyl) piperazin-1-yl) benzoic acid as a white solid. LC-MS: M+1=517. H-NMR: 6: 10.60 (bs, 1H), 7.73 (d, J=8.4 Hz, 1H), 7.42-7.39 (m, 2H), 7.14-7.11 (m, 3H), 6.95-6.92 (m, 1H), 4.00-3.84 (m, 2H), 3.76 (s, 2H), 3.51-3.33 (m, 4H), 2.79-2.60 (m, 2H), 2.32-2.30 (m, 2H), 2.03-1.97 (m, 2H), 1.47-1.45 (m, 2H), 0.97 (s, 6H).

Synthesis of 2-bromo-4-(4-((2-(4-chlorophenyl)-4, 4-dimethylcyclohex-1-enyl) methyl) piperazin-1-yl)-N-(3-nitro-4-((tetrahydro-2H-pyran-4-yl) methylamino) phenylsulfonyl) benzamide: Into a 20000-mL round-bottom flask, was placed 2-bromo-4-(4-((2-(4-chlorophenyl)-4, 4-dimethylcyclohex-1-enyl)methyl)piperazin-1-yl)benzoic acid (550 g, 1.07 mol, 1 equiv), DCM (10 L), 3-nitro-4-[[(oxan-4-yl)methyl]amino]benzene-1-sulfonamide (320 g, 1.02 mol, 0.95 equiv), EDCI (308 g, 1.61 mol, 1.5 equiv), DMAP (522 g, 4.28 mol, 4 equiv). The resulting solution was stirred for overnight at 25 degrees C. LCMS showed material was completely consumed. The resulting mixture is followed by dilute hydrochloric acid (1.0 M) (1000 mL×3), saturated sodium bicarbonate (1000 mL×3) and brine (1000 mL×1), and then the organic phase was dried by Na₂SO₄, filtrated. The filtrate was concentrated to give product 810 g (Y: 93%) as a light brown yellow solid 2-bromo-4-(4-((2-(4-chlorophenyl)-4, 4-dimethylcyclohex-1-enyl) methyl) piperazin-1-yl)-N-(3-nitro-4-((tetrahydro-2H-pyran-4-yl) methylamino) phenylsulfonyl) benzamide as a brown yellow solid. LC-MS: M+1=814. H-NMR: 6 8.63-8.61 (m, 2H), 7.94-7.92 (m, 1H), 7.37-7.35 (m, 3H), 7.27-7.24 (m, 1H), 7.05-7.02 (m, 3H), 6.86-6.83 (m, 1H), 3.87-3.82 (m, 2H), 3.37-3.23 (m, 8H), 2.92 (s, 2H), 2.50-2.38 (m, 4H), 2.22-2.20 (m, 2H), 2.00-1.97 (m, 2H), 1.64-1.60 (m, 2H), 1.48-1.46 (m, 2H), 1.26-1.20 (m, 2H), 0.97 (s, 6H).

Synthesis of 4-methyl-N-(3, 3, 3-trifluoro-2-hydroxypropyl)benzenesulfonamide: Into a 100-mL 3-necked round-bottom flask, was placed 3-amino-1, 1, 1-trifluoropropan-2-ol (950.00 mg, 7.364 mmol, 1.00 equiv), TEA (1.49 g, 14.729 mmol, 2.00 equiv), DCM (25.00 mL). This was followed by the addition of P-toluenesulfonyl chloride (1.40 g, 7.364 mmol, 1.00 equiv) dropwise with stirring at 0 degrees C. The resulting solution was stirred for 3h at room temperature in a water bath. The reaction was then quenched by the addition of 50 mL of water. The resulting solution was extracted with 3×50 mL of dichloromethane and the organic layers combined. The resulting mixture was washed with 2×50 ml of water and 1×50 mL of brine. The mixture was dried over anhydrous sodium sulfate and concentrated. The residue was applied onto a silica gel column with ethyl acetate/petroleum ether (1:1). This resulted in 2 g of 4-methyl-N-(3, 3, 3-trifluoro-2-hydroxypropyl) benzenesulfonamide as colorless oil. LC-MS: M+1=284.

Synthesis of N-[2-[(5-bromo-1-[[2-(trimethylsilyl) ethoxy] methyl] pyrrolo [2, 3-b] pyridin-6-yl) oxy]-3, 3, 3-trifluoropropyl]-4-methylbenzenesulfonamide: Into a 250-mL round-bottom flask, was placed 4-methyl-N-(3, 3, 3-trifluoro-2-hydroxypropyl) benzenesulfonamide (2.00 g, 7.067 mmol, 1.00 equiv), 5-bromo-6-fluoro-1-[[2-(trimethylsilyl) ethoxy] methyl] pyrrolo[2,3-b] pyridine (2.40 g, 7.067 mmol, 1.00 equiv), Cs₂CO₃ (4.60 g, 14.134 mmol, 2.00 equiv), Dioxane (50.00 mL). The resulting solution was stirred for 16h at 90 degrees C. in an oil bath. The reaction mixture was cooled to room temperature with a water bath. The reaction was then quenched by the addition of 50 mL of water. The solution was filtrated and filter cake was washed by EA (50 ml) three times. The resulting mixture was concentrated. The residue was applied onto a silica gel column with ethyl acetate/petroleum ether (1:3). This resulted in 2.7 g of N-[2-[(5-bromo-1-[[2-(trimethylsilyl) ethoxy] methyl] pyrrolo[2, 3-b] pyridin-6-yl) oxy]-3, 3, 3-trifluoropropyl]-4-methylbenzenesulfonamide as colorless oil. LC-MS: M+1=608.

Synthesis of 10-(4-methylbenzenesulfonyl)-12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene: Into a 100-mL round-bottom flask purged and maintained with an inert atmosphere of nitrogen, was placed N-[2-[(5-bromo-1-[[2-(trimethylsilyl)ethoxy]methyl]pyrrolo[2, 3-b]pyridin-6-yl) oxy]-3, 3, 3-trifluoropropyl]-4-methylbenzenesulfonamide (2.70 g, 4.448 mmol, 1.00 equiv), 2-(2-methylpropanoyl) cyclohexan-1-one (0.37 g, 2.224 mmol, 0.50 equiv), CuI (0.84 g, 4.448 mmol, 1.00 equiv), DMSO (50.00 mL), Cs₂CO₃ (4.30 g, 13.344 mmol, 3.00 equiv). The resulting solution was stirred for 2h at 120 degrees C. in an oil bath. The reaction mixture was cooled to room temperature with a water bath. The resulting solution was diluted with 200 mL of water. The resulting solution was extracted with 3×150 mL of ethyl acetate and the organic layers combined. The resulting mixture was washed with 3×100 ml of water and 1×100 mL of brine. The mixture was dried over anhydrous sodium sulfate and concentrated. The residue was applied onto a silica gel column with ethyl acetate/petroleum ether (1:3). This resulted in 2.3 g of 10-(4-methylbenzenesulfonyl)-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraene as light yellow oil. LC-MS: M+1=528.

Synthesis of 12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene: Into a 50-mL round-bottom flask purged and maintained with an inert atmosphere of nitrogen, was placed 10-(4-methylbenzenesulfonyl)-12-(trifluoromethyl)-4-[[2-(trimethylsilyl) ethoxy] methyl]-13-oxa-2, 4, 10-triazatricyclo[7.4.0.0\hat{ }[3,7]] trideca-1(9),2,5,7-tetraene (1.30 g, 2.467 mmol, 1.00 equiv), MeOH (10.00 mL), Mg (200.00 mg, 4.934 mmol, 2.00 equiv). The resulting solution was stirred for 18h at room temperatureh. The solids were filtered out. The resulting mixture was concentrated. The residue was applied onto a silica gel column with ethyl acetate/petroleum ether (1:3). This resulted in 700 mg of 12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene as colorless oil. LC-MS: M+1=374. H-NMR: 6 7.31-7.32 (d, J=3.0 Hz, 1H), 7.25 (s, 1H), 6.28-6.28 (d, J=3.0 Hz, 1H), 5.83 (s, 1H), 5.43 (s, 2H), 5.05-5.06 (d, J=3.0 Hz, 1H), 3.56-3.41 (m, 3H), 3.31-3.18 (m, 1H), 0.92-0.73 (m, 3H),-0.09 (s, 9H).

Synthesis of (12S)-12-(trifluoromethyl)-4-[[2-(trimethylsilyl) ethoxy] methyl]-13-oxa-2, 4, 10-triazatricyclo [7.4.0.0\hat{ }[3, 7]] trideca-1(9), 2, 5, 7-tetraene(Assumed): The 300 mg 12-(trifluoromethyl)-4-[[2-(trimethylsilyl) ethoxy] methyl]-13-oxa-2, 4, 10-triazatricyclo [7.4.0.0\hat{ }[3, 7]]trideca-1(9),2,5,7-tetraene was purified by Chiral-Prep-HPLC with the following conditions: Column, CHIRALPAK OJ-3, 50*4.6 mm, 3 um, OJ30CC-QK005; mobile phase A: n-Hexane, Mobile Phase B: Ethanol; Flow rate: 1.0 mL/min; Gradient: 0% B to 5% B in 6 min; Detector, 220 nm. This resulted in 130 mg (98%) of (12S)-12-(trifluoromethyl)-4-[[2-(trimethylsilyl) ethoxy] methyl]-13-oxa-2,4,10-triazatricyclo [7.4.0.0\hat{ }[3,7]]trideca-1(9),2,5,7-tetraene(Assumed). LC-MS: M+1=374. ee=99.95%.

Synthesis of 4-(4-[[2-(4-chlorophenyl)-4, 4-dimethylcyclohex-1-en-1-yl] methyl]piperazin-1-yl)-N-[3-nitro-4-[(oxan-4-ylmethyl)amino]benzenesulfonyl]-2-[(12S)-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide(Assumed): Into a 50-mL round-bottom flask purged and maintained with an inert atmosphere of nitrogen, was placed 2-bromo-4-(4-[[2-(4-chlorophenyl)-4, 4-dimethylcyclohex-1-en-1-yl] methyl]piperazin-1-yl)-N-[3-nitro-4-[(oxan-4-ylmethyl)amino]benzenesulfonyl]benzamide (140.00 mg, 0.172 mmol, 1.30 equiv), (12S)-12-(trifluoromethyl)-4-[[2-(trimethylsilyl) ethoxy]methyl]-13-oxa-2, 4, 10-triazatricyclo [7.4.0.0\hat{ }[3,7]] trideca-1(9),2,5,7-tetraene (Assumed) (50.00 mg, 0.133 mmol, 1.00 equiv), N₁,N₂-bis(4-hydroxy-2,6-dimethylphenyl)oxalamide (10.00 mg, 0.026 mmol, 0.20 equiv), Cs₂CO₃ (110.00 mg, 0.331 mmol, 2.50 equiv), CuI (10.00 mg, 0.053 mmol, 0.40 equiv), DMF (10.00 mL). The resulting solution was stirred for 2h at 100 degrees C. in an oil bath. The reaction mixture was cooled to room temperature with a water bath. The resulting solution was diluted with 60 ml of EA. The solids were filtered out. The mixture was dried over anhydrous sodium sulfate and concentrated. The residue was applied onto a silica gel column with dichloromethane/methanol (10:1). This resulted in 120 mg of 4-(4-[[2-(4-chlorophenyl)-4,4-dimethylcyclohex-1-en-1-yl]methyl]piperazin-1-yl)-N-[3-nitro-4-[(oxan-4-ylmethyl)amino]benzenesulfonyl]-2-[(12S)-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide(Assumed) as a yellow solid. LC-MS: M+1=1107.

Synthesis of 4-(4-[[2-(4-chlorophenyl)-4, 4-dimethylcyclohex-1-en-1-yl] methyl]piperazin-1-yl)-N-[3-nitro-4-[(oxan-4-ylmethyl)amino]benzenesulfonyl]-2-[(12S)-12-(trifluoromethyl)-13-oxa-2,4,10-triazatricyclo[7.4.0.0\hat{ }[3,7]]trideca-1(9),2,5,7-tetraen-10-yl]benzamide(Assumed): Into a 50-mL round-bottom flask, was placed 4-(4-[[2-(4-chlorophenyl)-4, 4-dimethylcyclohex-1-en-1-yl] methyl] piperazin-1-yl)-N-[3-nitro-4-[(oxan-4-ylmethyl) amino] benzenesulfonyl]-2-[(12S)-12-(trifluoromethyl)-4-[[2-(trimethylsilyl)ethoxy]methyl]-13-oxa-2,4,10-triazatricyclo[7.4.0.0\hat{ }[3,7]]trideca-1(9),2,5,7-tetraen-10-yl]benzamide(Assumed) (120.00 mg, 0.108 mmol, 1.00 equiv), ethylenediamine (1.00 mL), THE (5.00 mL), TBAF.3H₂O (43.00 mg, 0.163 mmol, 1.50 equiv). The resulting solution was stirred for 72h at 80 degrees C. in an oil bath. The reaction mixture was cooled to room temperature with a water bath. The resulting solution was diluted with 10 mL of water. The resulting solution was extracted with 3×10 mL of ethyl acetate and the organic layers combined. The resulting mixture was washed with 1×10 ml of brine. The mixture was dried over anhydrous sodium sulfate and concentrated. The residue was applied onto a silica gel column with dichloromethane/methanol (5:1). The crude product was purified by Prep-HIPLC with the following conditions: column, X-Bridge Prep C18 19*150 mm Sum; mobile phase, A: water (it contains 10 mM NH₄HCO₃ 0.05% ammonia); B: ACN; Gradient: 20-45% B in 8 min; Flow rate: 20 mL/min; detector, UV 220 nm. The collected solution was concentrated under vacuum to remove CH₃CN and the resulting solution was dried by lyophilization. This resulted in 11 mg of 4-(4-[[2-(4-chlorophenyl)-4, 4-dimethylcyclohex-1-en-1-yl] methyl]piperazin-1-yl)-N-[3-nitro-4-[(oxan-4-ylmethyl) amino]benzenesulfonyl]-2-[(12S)-12-(trifluoromethyl)-13-oxa-2,4,10-triazatricyclo[7.4.0.0\hat{ }[3,7]]trideca-1(9),2,5,7-tetraen-10-yl]benzamide(Assumed) as a yellow solid. LC-MS: M+1=977. ee=99.99%. H-NMR: (300 MHz, DMSO-d₆): δ 11.06 (s, 1H), 8.48 (s, 1H), 8.34 (s, 1H), 7.47-7.50 (d, J=9.0 Hz, 1H), 7.35-7.38 (d, J=9.0 Hz, 2H), 7.06-7.19 (m, 3H), 6.77-6.83 (m, 2H), 6.52-6.64 (m, 1H), 5.98 (s, 1H), 5.13 (s, 1H), 3.85-3.90 (m, 2H), 3.42-3.80 (m, 2H), 3.22-3.32 (m, 6H), 2.71-2.89 (m, 2H), 2.19-2.28 (m, 6H), 1.99 (s, 3H), 1.80-1.92 (m, 1H), 1.62-1.66 (m, 2H), 1.41-1.43 (m, 2H), 1.24-1.30 (m, 6H), 0.95 (s, 6H).

### Part B:

Compounds believed to be particularly useful in preparation of solid dispersions of the invention include without limitation:
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((5aS,8aR)-5a,6,8,8a-tetrahydrofuro[3,4-b]pyrrolo[3′,2′:5,6]pyrido[3,2-e][1,4]oxazin-5(1H)-yl)benzamide,
(R)-4-(4-((4′-chloro-2′-fluoro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-2-(4-methyl-3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(4-methyl-3,4-dihydro-2H-pyrrolo[3′,2′:5,6]pyrido[2,3-b][1,4]oxazepin-1(7H)-yl)benzamide,
N-((4-((((R)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-methyl-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-methyl-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-methyl-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-N-((4-(((1,4-dioxan-2-yl)methyl)amino)-3-nitrophenyl)sulfonyl)-4-(4-((4′-chloro-4,4-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(S)-N-((4-(((1,4-dioxan-2-yl)methyl)amino)-3-nitrophenyl)sulfonyl)-4-(4-((4′-chloro-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((S)-5-nitro-3-(tetrahydro-2H-pyran-4-yl)-3,4-dihydro-2H-benzo[b][1,4]oxazin-7-yl)sulfonyl)benzamide,
4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)-N-(((R)-5-nitro-3-(tetrahydro-2H-pyran-4-yl)-3,4-dihydro-2H-benzo[b][1,4]oxazin-7-yl)sulfonyl)benzamide,
N-(((R)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
N-(((S)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
(S)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((5-nitro-3-(tetrahydro-2H-pyran-4-yl)-3,4-dihydro-2H-benzo[b][1,4]oxazin-7-yl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((5-nitro-3-(tetrahydro-2H-pyran-4-yl)-3,4-dihydro-2H-benzo[b][1,4]oxazin-7-yl)sulfonyl)benzamide,
N-(((R)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
N-(((S)-3-((S)-1,4-dioxan-2-yl)-5-nitro-3,4-dihydro-2H-benzo[b][1,4]oxazin-7-yl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,

### Part C. Compound Forms

In some embodiments, a compound of Formula A is present in the solid dispersion in its parent-compound form, alone or together with a salt form of the compound.

Compounds of Formula A may form acid addition salts, basic addition salts or zwitterions. Salts of compounds of Formula A can be prepared during isolation or following purification of the compounds.

Acid addition salts are those derived from reaction of a compound of Formula I with an acid. For example, salts including the acetate, adipate, alginate, ascorbate, bicarbonate, citrate, aspartate, benzoate, benzenesulfonate (besylate), bisulfate, butyrate, camphorate, camphorsulfonate, digluconate, ethanedisulfonate, formate, fumarate, glycerophosphate, glutamate, hemisulfate, heptanoate, hexanoate, hydrobromide, hydrochloride, hydroiodide, l-hydroxy-2-naphthoate, lactate, lactobionate, malate, maleate, malonate, mesitylenesulfonate, methanesulfonate, naphthalenesulfonate, nicotinate, oxalate, pamoate, pectinate, persulfate, phosphate, picrate, propionate, succinate, sulfate, tartrate, thiocyanate, trichloroacetate, trifluoroacetate, para-toluenesulfonate and undecanoate salts of a compound of Formula I can be used in a composition of the invention.

Basic addition salts, including those derived from reaction of a compound with the bicarbonate, carbonate, hydroxide or phosphate of cations such as lithium, sodium, potassium, calcium and magnesium, can likewise be used.

A compound of Formula A typically has more than one protonatable nitrogen atom and is consequently capable of forming acid addition salts with more than one, for example about 1.2 to about 2, about 1.5 to about 2 or about 1.8 to about 2, equivalents of acid per equivalent of the compound.

Without being bound by theory, it is believed that the therapeutic efficacy of compounds of Formula A is due at least in part to their ability to bind to a Bcl-2 family protein such as Bcl-2, or Bcl-XL in a way that inhibits the anti-apoptotic action of the protein, for example by occupying the BH3 binding groove of the protein. It will generally be found desirable to select a compound having high binding affinity for a Bcl-2 family protein.

A compound of Formula A or a salt thereof is present in a solid dispersion of the invention in an amount that can be therapeutically effective when the composition is administered to a subject in need thereof according to an appropriate regimen. Dosage amounts are expressed herein as parent-compound-equivalent amounts unless the context requires otherwise. Typically, a unit dose (the amount administered at a single time), which can be administered at an appropriate frequency, e.g., twice daily to once weekly, is about 10 to about 1,000 mg, depending on the compound in question. Where frequency of administration is once daily (q.d.), unit dose and daily dose are the same. Illustratively, the unit dose is typically about 25 to about 1,000 mg, more typically about 50 to about 800 mg, for example about 50, about 100, about 150, about 200, about 250, about 300, about 350, about 400, about 450, about 500 mg, about 550 mg, about 600 mg, about 650 mg, about 700 mg, about 750 mg, or about 800 mg. Where the dosage form comprises a capsule shell enclosing the solid dispersion, or a tablet wherein the solid dispersion is formulated with other ingredients, a unit dose can be deliverable in a single dosage form or a plurality of dosage forms, most typically 1 to about 10 dosage forms.

### Part D. Formulations

The higher the unit dose, the more desirable it becomes to prepare a solid dispersion having a relatively high concentration of the drug therein. Typically, the concentration of drug in the solid dispersion is at least about 1%, e.g., about 1% to about 50%, by parent-compound-equivalent weight, but lower and higher concentrations can be acceptable or achievable in specific cases. Illustratively, the drug concentration in various embodiments is at least about 2%, e.g., about 2% to about 50%>, or at least about 5%, e.g., about 5%> to about 40%, for example about 5%, about 10%, about 15%, about 20%, about 25%), about 30%), about 35% or about 40%, by parent-compound-equivalent weight. In some embodiments, the drug concentration may be between about 5% and about 25%, such as between about 5% and about 25%, such as about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%.

The major component of the matrix of a solid dispersion product is a polymer that is hydrophilic or water-soluble at least in a part of the pH scale, more particularly at a pH occurring in the gastrointestinal (GI) tract, or a combination of such polymers. A polymer or polymer mixture useful herein is solid at ambient temperature and, in the interests of good storage stability at a range of temperatures, should remain solid even at the highest temperatures typically experienced during storage, transport and handling of the product. A useful property of a polymer determining its usefulness herein is therefore its glass transition temperature (T_{g}). Suitable water-soluble polymers include, but are not limited to, those having a T_{g} of at least about 40° C., at least about 50° C., at least about 60° C., or more, and more particularly about 80° C. to about 180° C. Methods for determining T_{g} values of organic polymers are described for example in Sperling, ed. (1992) Introduction To Physical Polymer Science, 2nd edition, John Wiley & Sons, Inc.

Polymers are considered water-soluble if they form a clear homogeneous solution in water (e.g., a solution that is essentially uniform throughout, and that appears clear under visual inspection or alternatively using an instrument such as a turbidimeter, the solution for example exhibiting little or no scattering of a light beam going therethrough). When dissolved at 20° C. in an aqueous solution at 2% (w/v), a suitable water-soluble polymer illustratively has an apparent viscosity of about 1 to about 5000 mPa·s, for example about 1 to about 700 mPa·s, or about 5 to about 100 mPa·s. Water-dispersable or water-swellable polymers can also be used. Non-limiting examples of polymeric carriers useful herein include:
homopolymers and copolymers of N-vinyl lactams, especially homopolymers and copolymers of N-vinyl pyrrolidone, e.g., the homopolymer polyvinylpyrrolidone (PVP or povidone) and copolymers such as those comprising monomers of N-vinyl pyrrolidone and vinyl acetate (copovidone) or N-vinyl pyrrolidone and vinyl propionate;
cellulose esters and cellulose ethers, in particular methylcellulose, ethylcellulose, (hydroxyalkyl)celluloses such as hydroxypropylcellulose, (hydroxyalkyl)alkyl-celluloses such as hydroxypropylmethylcellulose (HPMC or hypromellose), cellulose phthalates and succinates such as cellulose acetate phthalate, hydroxypropylmethylcellulose phthalate (HPMC-P), hydroxypropylmethylcellulose succinate (HPMC-S) and hydroxypropylmethylcellulose acetate succinate (HPMC-AS);
high molecular weight polyalkylene oxides such as polyethylene oxides (PEGs or PEOs) and copolymers of ethylene oxide and propylene oxide (poloxamers);
polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates) and poly(hydroxyalkyl methacrylates);
polyacrylamides;
vinyl acetate polymers such as copolymers of vinyl acetate and crotonic acid, polyvinyl acetate, polyvinyl alcohol and partially hydrolyzed polyvinyl acetate (also referred to as partially saponified polyvinyl alcohol);
graft copolymers of polyethylene glycol, polyvinyl caprolactam and polyvinyl acetate (e.g., Soluplus™ of BASF or equivalent product);
oligo- and polysaccharides such as carrageenans, galactomannans and xanthan gum;
and mixtures of two or more thereof.

Suitable povidones include, without limitation, those having a K-value (a measure of viscosity of an aqueous solution of the povidone) of about 12, about 15, about 17, about 25, about 30 or about 90, and mixtures thereof. A particular example of a useful povidone is povidone (or PVP) K30.

Another suitable polymer is a mixture of PVP and polyvinyl acetate such as that sold under the name KOLLIDON® SR by BASF AG. In some embodiments, a vinylpyrrolidone-vinyl acetate copolymer commercially available as KOLLIDON® VA 64, which is soluble both in water and in alcohol, is suitable in the formulations of the present invention.

A particular example of a useful copovidone is one consisting of about 60% N-vinyl pyrrolidone and about 40% vinyl acetate monomers, referred to herein as “copovidone 60/40”.

Suitable HPMCs and derivatives thereof include, without limitation, IPMC E3, IPMC E5, IPMC E6, HPMC E15, IPMC K3, IPMC A4, IPMC A15, HPMC-AS LF, HPMC-AS MF, HPMC-AS HF, HPMC-AS LG, HPMC-AS MG, HPMC-AS HG, HPMC-P 50, HPMC-P 55 and mixtures thereof.

Suitable ethylcelluloses include, without limitation, those sold under the Ethocel™ trademark of Dow Chemical Company as Ethocel™ 4, Ethocel™ 7, Ethocel™ 10, Ethocel™ 14 and Ethocel™ 20, products of other manufacturers equivalent thereto, and mixtures thereof.

Suitable methacrylic acid/methyl methacrylate copolymers include, without limitation, those sold under the Eudragit™ trademark of Rohm GmbH as Eudragit™ LI 00, Eudragit™ L100-55 and Eudragit™ S100, products of other manufacturers equivalent thereto, and mixtures thereof.

Suitable PEGs include, without limitation, PEG 400, PEG 600, PEG 1450, PEG 3350, PEG 4000, PEG 6000, PEG 8000, PEG 20000 and mixtures thereof. Lower molecular-weight PEGs such as PEG 400 and PEG 600 may be unsuitable as the sole polymeric carrier but can be useful in combination with other polymers.

Suitable poloxamers include, without limitation, poloxamer 124, poloxamer 188, poloxamer 237, poloxamer 338, poloxamer 407 and mixtures thereof. It will be noted that poloxamers have surfactant properties and can be included primarily for these properties as well as their polymeric nature.

In one embodiment, the solid dispersion matrix comprises one or more polymeric carriers selected from the group consisting of povidones, copovidones, HPMCs, polyethylene glycol/polyvinyl caprolactam/polyvinyl acetate graft copolymers and mixtures thereof. In a more particular embodiment, the solid dispersion matrix comprises one or more polymeric carriers selected from the group consisting of povidone K30, copovidone 60/40, HPMC E5, Soluplus™ polyethylene glycol/polyvinyl caprolactam/polyvinyl acetate graft copolymer and products equivalent to Soluplus™ One or more polymeric carriers typically constitute in total about 20% to about 95%, such as about 20% to about 90%, for example about 40% to about 85%, or about 60% to about 85%, or about 70% to about 85%, or even about 75% to about 85%, by weight of the solid dispersion.

Upon oral administration and exposure to GI fluid, it is believed without being bound by theory that, through interplay between the polymeric carrier and a surfactant component of the solid dispersion, a suitable release rate and inhibition of crystallization or recrystallization of the active ingredient are provided, thereby permitting bioabsorption.

The surfactant component can be anionic, non-ionic or can comprise a combination of anionic and non-ionic surfactants. Useful pharmaceutically acceptable anionic surfactants include alkyl sulfates (e.g., sodium lauryl sulfate), alkylcarboxylates, alkylbenzole sulfates and secondary alkane sulfonates.

Particularly useful as surfactants herein are pharmaceutically acceptable non-ionic surfactants, especially those having a hydrophilic-lipophilic balance (HLB) value of about 8 to about 29, for example about 9 to about 17, or about 11 to about 15. The HLB system (see Fiedler (2002) Encyclopedia of Excipients, 5th edition, Aulendorf: ECV-Editio-Cantor-Verlag) assigns numeric values to surfactants, with lipophilic substances receiving lower HLB values and hydrophilic substances receiving higher HLB values. Non-limiting examples of non-ionic surfactants useful herein include:
polyoxyethylene castor oil derivatives such as PEG-35 castor oil (e.g., Cremophor EL™ of BASF or equivalent product), PEG-40 hydrogenated castor oil (e.g., Cremophor RH™ 40 or equivalent product) and PEG-60 hydrogenated castor oil (e.g., Cremophor RH™ 60 or equivalent product);
other polyoxyethylene glycerides such as PEG-32 glyceryl laurate (e.g., Gelucire™ 44/14 of Gattefosse or equivalent product) and PEG-32 glyceryl palmitostearate (e.g., Gelucire™ 50/13 or equivalent product), and Labrafil Ml 944 CS (oleoyl macrogol 6 glycerides prepared by transesterification of apricot kernel oil with PEG 300);
fatty acid monoesters of sorbitan, for example sorbitan monooleate (e.g., Span™ 80 or equivalent product), sorbitan monostearate (e.g., Span™ 60 or equivalent product), sorbitan monopalmitate (e.g., Span™ 40 or equivalent product) and sorbitan monolaurate (e.g., Span™ 20 or equivalent product);
other fatty acid esters of sorbitan, for example, sorbitan tristearate and sorbitan trioleate;
fatty acid monoesters of polyoxyethylene sorbitan (polysorbates) such as PEG-20 sorbitan monooleate (polysorbate 80, e.g., Tween™ 80 or equivalent product) PEG-20 sorbitan monostearate (polysorbate 60, e.g., Tween™ 60 or equivalent product), PEG-20 sorbitan monopalmitate (polysorbate 40, e.g., Tween™ 40 or equivalent product), or PEG-20 sorbitan monolaurate (polysorbate 20, e.g., Tween™ 20 or equivalent product);
other fatty acid esters of polyoxyethylene sorbitan, for example, polyoxyethylene (20) sorbitan tristearate (Tween 65), polyoxyethylene (20) sorbitan trioleate (Tween 85);
fatty acid ester of polyalkylene glycols such as, for example, PEG 660 hydroxy-stearic acid (polyglycol ester of 12-hydroxystearic acid (70 mol %) with 30 mol % ethylene glycol);
polyalkoxylated ethers of fatty alcohols such as, for example, PEG (2) stearyl ether (Brij 72), macrogol 6 cetylstearyl ether or macrogol 25 cetylstearyl ether;
A tocopheryl compound, for example, a-tocopheryl polyethylene glycol succinate, which is commonly abbreviated as vitamin E-TPGS. Vitamin E-TPGS is a water-soluble form of natural-source vitamin E prepared by esterifying d-alpha-tocopheryl acid succinate with polyethylene glycol 1000;
and mixtures of two or more thereof.

In one embodiment, the solid dispersion comprises one or more surfactants selected from the group consisting of polyoxyethylene glycerides (including polyoxyethylene castor oil derivatives), polysorbates, TPGS and mixtures thereof. In a more particular embodiment, the solid dispersion matrix comprises one or more polymeric carriers selected from the group consisting of PEG-40 hydrogenated castor oil, polysorbate 80, polysorbate 20 and TPGS.

One or more surfactants typically constitute in total about 2% to about 25%, for example about 5% to about 20%, such as between about 5% and about 15%, or between about 5% and about 10% by weight of the solid dispersion.

One surprisingly beneficial component of the matrix of a solid dispersion product is an antioxidant. Antioxidants are compounds that normally inhibit oxidation which is a chemical reaction that can produce free radicals, thereby leading to chain reactions that may damage the cells of organisms. Antioxidants such as thiols or ascorbic acid (vitamin C) terminate these chain reactions.

In one embodiment, the solid dispersion comprises one or more pharmaceutically acceptable antioxidant such as ascorbic acid, an ascorbate, a bisulfite, a metabisulfate, a sulfite, curcumin, curcumin derivatives, ursolic acid, resveratrol, resveratrol derivatives, alpha-lipoic acid, thioglycerol, a polyphenol, catachins, grapeseed extract, green tea extract, citric acid, methionine, cysteine, glutathione, tocopherol, propyl gallate, sodium mercaptoacetate, sodium formaldehyde sulfoxylate, ascorbyl palmitate, butylated hydroxyanisole, butylated hydroxytoluene, lecithin, vitamin E, uric acid, and/or mixtures thereof.

In certain embodiments, the one or more antioxidant typically constitute in total w/w % of about 0.1% to about 5%, for example about 0.25% to about 2.5%, or 0.5% to about 2.5% by weight of the solid dispersion.

Other, optional, components of the solid dispersion include, without limitation, one or more lubricants, glidants or flow regulators. Such a material, for example colloidal silicon dioxide or fumed silica (e.g., Aerosil), can reduce adhesion of the extrudable mixture to the screw and wall of the extruder and can facilitate the smooth passage of extrudate through the die. Illustratively, colloidal silicon dioxide can constitute zero to about 5%, for example about 0.1% to about 2%, by weight of the solid dispersion. The solid dispersion can also optionally contain one or more bulking agents (fillers), disintegrants, cosolvents such as propylene glycol esters of fatty acids (e.g., propylene glycol laurate), plasticizers and/or stabilizers such as antioxidants, light stabilizers, free radical scavengers or antimicrobial agents.

A dosage form of the invention can consist of, or consist essentially of, a solid dispersion as described above. However, in some embodiments a dosage form contains additional excipients and requires additional processing of the solid dispersion. For example, the solid dispersion can be ground to a powder and filled into a capsule shell or molded or compressed to form a tablet, with additional excipients as may be conventionally used in such dosage forms.

Thus orally deliverable solid dosage forms of the invention include but are not limited to capsules, dragees, granules, pills, powders and tablets. Excipients commonly used to formulate such dosage forms include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, preservatives, propellants, releasing agents, sterilizing agents, sweeteners, solubilizers and mixtures thereof. Many excipients have two or more functions in a pharmaceutical composition. Characterization herein of a particular excipient as having a certain function, e.g., diluent, disintegrant, binding agent, etc., should not be read as limiting to that function. Further information on excipients can be found in standard reference works such as Handbook of Pharmaceutical Excipients, 3rd ed. (Kibbe, ed. (2000), Washington: American Pharmaceutical Association).

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches {e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of a- and amorphous cellulose and powdered cellulose, and cellulose acetate; calcium salts including calcium carbonate, tribasic calcium phosphate, dicalcium phosphate (e.g., dibasic calcium phosphate dihydrate), monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 1% to about 95%, for example about 5% to about 50%, or about 10%> to about 30%>, by weight of the composition. The diluent or diluents selected preferably exhibit suitable flow properties and, where tablets are desired, compressibility.

Microcrystalline cellulose and silicified microcrystalline cellulose are particularly useful diluents, and are optionally used in combination with a water-soluble diluent such as mannitol. Illustratively, a suitable weight ratio of microcrystalline cellulose or silicified microcrystalline cellulose to mannitol is about 10: 1 to about 1: 1, but ratios outside this range can be useful in particular circumstances.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.5% to about 20%, or about 1%) to about 10%), by weight of the composition.

Sodium starch glycolate is a particularly useful disintegrant, and typically constitutes in total about 1% to about 20%, for example about 2% to about 15%, or about 5% to about 10%), by weight of the composition.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone or PVP), for example povidone K-15, K-30 and K-29/32; polyacrylic acids (carbomers); polymethacrylates; and the like. One or more binding agents and/or adhesives, if present, typically constitute in total about 0.5% to about 25%, for example about 1% to about 15%, or about 1.5% to about 10%, by weight of the composition.

Povidone and hydroxypropylcellulose, either individually or in combination, are particularly useful binding agents for tablet formulations, and, if present, typically constitute about 0.5%) to about 15%, for example about 1% to about 10%, or about 2% to about 8%, by weight of the composition.

Wetting agents can be added to the formulation if desired, in addition to the surfactant component of the solid dispersion. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters, for example sorbitan monolaurate, sorbitan monooleate, sorbitan monopalmitate and sorbitan monostearate; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; a-tocopherol polyethylene glycol (1000) succinate (TPGS); tyloxapol; and the like. One or more wetting agents, if present, typically constitute in total about 0.1% to about 15%, for example about 0.2%> to about 10%>, or about 0.5%> to about 7%, by weight of the composition, excluding surfactant present in the solid dispersion.

Nonionic surfactants, more particularly poloxamers, are examples of wetting agents that can be useful herein. Illustratively, a poloxamer such as Pluronic™ F127, if present, can constitute about 0.1% to about 10%>, for example about 0.2%> to about 7%, or about 0.5%) to about 5%, by weight of the composition, excluding surfactant present in the solid dispersion.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 5%, or about 0.2% to about 2%, by weight of the composition. Sodium stearyl fumarate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.05% to about 10%, for example about 0.1%) to about 7%, or about 0.2% to about 5%, by weight of the composition. Colloidal silicon dioxide is a particularly useful anti-adherent.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.05% to about 10%, for example about 0.1% to about 7%, or about 0.2% to about 5%, by weight of the composition, excluding glidant present in the solid dispersion. Colloidal silicon dioxide is a particularly useful glidant.

Other excipients such as buffering agents, stabilizers, antioxidants, antimicrobials, colorants, flavors and sweeteners are known in the pharmaceutical art and can be used in compositions of the present invention. Tablets can be uncoated or can comprise a core that is coated, for example with a nonfunctional film or a release-modifying or enteric coating. Capsules can have hard or soft shells comprising, for example, gelatin (in the form of hard gelatin capsules or soft elastic gelatin capsules), starch, carrageenan and/or HPMC, optionally together with one or more plasticizers.

### Part E. Method of Preparation

As indicated above, the solid dispersion of the present invention can be prepared by a process comprising melt-extrusion. Accordingly, the solid dispersion product is a melt-processed, solidified mixture. The melt-extrusion process comprises the steps of preparing a homogeneous melt of the active ingredient or the combination of active ingredients, the pharmaceutically acceptable polymer and the solubilizers, and cooling the melt until it solidifies. “Melting” refers to a transition from a solid to a liquid (or alternatively from a solid to a softened or rubbery state), such that it is possible for one component to become homogeneously mixed with or embedded in the other. Typically, one component will melt and the other components will dissolve in the melt, thus forming a solution. In accordance with the present disclosure, melting generally involves heating above the softening point of the pharmaceutically acceptable polymer. The preparation of the melt can take place in a variety of ways. The mixing of the components can take place before, during or after the formation of the melt. For example, the components can be mixed first and then melted or simultaneously mixed and melted. Usually, the melt is homogenized in order to disperse the active ingredients efficiently. Also, it may be convenient first to melt the pharmaceutically acceptable polymer and then to admix and homogenize the active ingredients.

In a first step of this process, at least three components are subjected to elevated temperature to provide an extrudable mixture: a compound of Formula I or a pharmaceutically acceptable salt thereof; at least one pharmaceutically acceptable water-soluble polymeric carrier; and at least one pharmaceutically acceptable surfactant. Other components can optionally be included at this stage, for example one or more additional active ingredients and/or one or more additional excipients, for example a glidant.

The components can be mixed together before, during or after melting or softening of the polymeric carrier. In one embodiment, the components are mixed first and then subjected to elevated temperature to form the extrudable mixture. In another embodiment, the components are mixed while at elevated temperature to form the extrudable mixture. In yet another embodiment the polymeric carrier is first melted or softened, optionally with the surfactant component, and the compound of Formula I or salt thereof is then added to the resulting melt. In yet another embodiment, the surfactant is added to the polymer/API melt. According to all these embodiments, it will be found preferable to thoroughly mix the melt while at elevated temperature in order to ensure homogeneous dispersion or dissolution of the active ingredient in the partially or completely molten matrix. In one embodiment, the polymeric carrier, the surfactant, and optionally the glidant are mixed first into a pre-blend. The API is then added and the mixture is blended, in order to achieve a sufficiently homogeneous blend.

The melting and mixing takes place in an apparatus customary for this purpose. Particularly suitable are extruders or kneaders. Suitable extruders include single-screw extruders, intermeshing screw extruders and multiscrew extruders, for example twin-screw extruders, which can be co-rotating or counter-rotating and, optionally, equipped with kneading disks or other screw elements for mixing or dispersing the components of the melt. In some preferred embodiments, a single screw extruder may be used for small (e.g., benchtop) and large scale (commercial) production of extrudates.

The elevated temperature attained during this part of the process can suitably be about 70° C. to about 250° C., preferably 80° C. to about 250° C., such as for example about 80° C. to about 180° C., about 100° C. to about 180° C., or about 90° C. to about 160° C., or 120° C. to about 160° C. Illustratively the temperature at the feeding end of the extruder is maintained at a relatively moderate temperature, for example about 80° C. to about 120° C., about 80° C. to about 110° C., or about 90° C. to about 100° C.; in the middle portion of the extruder at a higher temperature, for example about 130° C. to about 180° C., or about 140° C. to about 160° C.; and near the extrusion die at a somewhat lower temperature, for example about 110° C. to about 160° C., or about 120° C. to about 150° C. It will be appreciated that appropriate working temperatures are determined in part by the kind of extruder and the configuration of elements within the extruder used. Part of the energy needed to melt, mix and dissolve the components in the extruder can be provided by heating elements. However, friction and shearing of the material in the extruder can also impart a substantial amount of thermal energy to the mixture and aid in the formation of a homogeneous melt of the components.

Other process parameters such as screw speed and feed rate will depend on the particular composition and equipment used and can readily be optimized by one of ordinary skill in the art without undue experimentation.

An API such as a compound of Formula A or a salt thereof can be added as a solid powder or in solution or dispersion in a suitable liquid solvent such as an alcohol or aliphatic hydrocarbon or ester, or liquid carbon dioxide. Such solvent is removed, e.g., evaporated, in preparation of the melt.

The extrudate emerging from the extruder die is semi-solid, for example pasty or viscous in texture. Before cooling to ambient temperature, the extrudate can, if desired, be shaped into any desired shape, for example by use of a calender with two counter-rotating rollers having mutually matching depressions on their surface. Typically such a procedure results in “lentils” (small lenticular tablets). If the rollers do not have depressions on their surfaces, films can be obtained. In one embodiment, extrudate is fed directly from the extrusion equipment to the calender. Alternatively, the extrudate can molded into a desired shape by injection-molding, or simply cut into pieces, either before solidification (hot-cut) or after solidification (cold-cut).

In a variant of the above process, a foam can be formed if the extrudate contains a propellant such as a gas, e.g., carbon dioxide, or volatile compound, e.g., a low molecular-weight hydrocarbon, or a compound that is thermally decomposable to a gas. The propellant is dissolved in the extrudate under the relatively high pressure conditions within the extruder and, when the extrudate emerges from the die, the sudden release of pressure causes the propellant to come out of solution and vaporize, resulting in formation of a foam.

According to empirical results to-date, it has been observed that the manufacturability and quality of the extrudates may be affected and improved by the selection of matrix polymer and surfactant, as well as the percentage of API (i.e. drug load) in the extrusion blend. In general, the maximum drug load in a desirably clear, homogenous extrudate may be improved via the selection of polymer and surfactant in the extrusion blend and selection of the concentrations of the API, the polymer, and the surfactant in the extrusion blend. In these or still other preferred embodiments, the API content in the extrusion blend may range from about 1% to about 25%, preferably about 5% to 25%, most preferably about 7.5% to about 25%).

In some embodiments of the invention, copovidone (KOLLIDON® VA64) or Soluplus is used as polymer carrier, at a concentration of for example about 50% to about 90%, about 55% to about 80%, or about 58% to 72%.

In these or other embodiments of the invention, the surfactant (e.g., Vitamin E TPGS, Tween20, Tween80/Polysorbate 80, poloxamer F68NF, Span 20, Sucroester WP15, Lauroglycol FCC, SDS, or PEG 400) may be employed at about 2% to about 15%, preferably about 4% to about 10%, most preferably about 5% to about 7% level.

In these or still other embodiments, the antioxidant content (e.g., in the extrusion blend or in the final formulation) may range from about 0.1% to about 5%, about 0.25% to 2.5%, or about 0.5% to about 2.5%).

Optionally, glidant (e.g., Aerosil) can also be included in the extrusion blend at about 0% to about 3%, preferably about 0.5% to about 1.8% level.

In this regard, it is to be noted that, in one or more embodiments, it may be desirable to control the weight % ratio of two or more of the components (e.g., the API and the surfactant) of the extrusion blend, in order for example to optimize the processability of the blend (e.g., to optimize the physical properties of the blend for manufacturing purposes, such as degradation, glass transition temperature (Tg), release time, etc.). Thus, these ratios may vary as a function of, for example, the process conditions and/or components utilized in the extrusion blend. However, in various exemplary embodiments, the weight % ratio of, for example, the API to surfactant may range from about 15:1 to about 1:15, from about 10:1 to about 1:10, from about 5:1 to about 1:5, from about 4:1 to about 1:4, or from about 2:1 to about 1:2 (e.g., about 2:1,about 1.5:1,about 1:1,about 1:1.5 or about 1:2. In this regard it is to be further noted that, in these or yet additional embodiments, two or more APIs (e.g., two or more compounds falling within the scope of Formula I, or alternatively a compound falling within the scope of Formula I and another compound or active, such as another chemotherapeutic compound disclosed elsewhere herein) may be utilized in the extrusion blend (provided, for example, the compounds are suitable stable under the processing conditions and there is little or no interaction between the compounds that would lead to chemical instability of the compounds).

In this regard it is to be still further noted that selection or choice of the polymer carrier may impact various performance properties of the resulting extrudate (or composition).

In certain embodiments, a formulation of the invention comprises a compound of Formula (A), such as any compound disclosed herein, in either a 10 mg or 100 mg table with the following specific compositions, including w/w % variations that are within 5% of the respective values (e.g., 59.88 wt %+2.994 wt %), is provided below.

### Exemplary Composition of a 10 mg Tablet for a Compound of Formula (A)

| | Unit Formula | | | |
|---|---|---|---|---|
| | mg per | %, | Quality | |
| Components | unit | w/w | Reference | Function |

Intra-granular Ingredients

| | | | | |
|---|---|---|---|---|
| Compound of Formula (A) | 10.00 | 8.98 | In-house | Active |
| | | | | ingredient |
| KOLLIDON ® VA64 | 66.67 | 59.88 | USP | Solubilizer |
| Sodium Ascorbate | 0.80 | 0.72 | USP | Anti-oxidant |
| PB80 | 5.83 | 5.24 | USP | surfactant |
| Colloidal Silicon Dioxide | 0.83 | 0.75 | USP | glidant |
| Total Intra-granules(mg) | 84.13 | — | — | — |

Extra-granular Ingredients

| | | | | |
|---|---|---|---|---|
| Croscarmellose Sodium | 21.32 | 19.15 | USP | Disintegrant |
| Colloidal Silicon Dioxide | 1.06 | 0.95 | USP | glidant |
| Sodium Stearyl Fumarate | 0.53 | 0.48 | USP | Lubricant |
| Core tablet Weight (mg) | 107.05 | — | — | — |

Film Coating Ingredients

| | | | | |
|---|---|---|---|---|
| Opadry ® II 85F92209-CN | 4.28 | 3.85 | In-house | Film Coating |
| Yellow | | | | agent |
| Purified Water | q.s. | — | USP | Solvent |
| Total tablet weight(mg) | 111.33 | 100.00 | | |

| | Unit Formula | | | |
|---|---|---|---|---|
| | mg per | %, | Quality | |
| Components | unit | w/w | Reference | Function |

Intra-granular Ingredients

| | | | | |
|---|---|---|---|---|
| Compound of Formula (A) | 100.00 | 8.98 | In-house | Active |
| | | | | ingredient |
| KOLLIDON ® VA64 | 666.67 | 59.88 | USP | Solubilizer |
| Sodium Ascorbate | 8.00 | 0.72 | USP | Anti-oxidant |
| Polysorbate 80 | 58.33 | 5.24 | USP | surfactant |
| Colloidal Silicon Dioxide | 8.33 | 0.75 | USP | glidant |
| Total Intra-granules(mg) | 841.33 | — | — | — |

Extra-granular Ingredients

| | | | | |
|---|---|---|---|---|
| Croscarmellose Sodium | 213.20 | 19.15 | USP | Disintegrant |
| Colloidal Silicon Dioxide | 10.63 | 0.95 | USP | glidant |
| Sodium Stearyl Fumarate | 5.33 | 0.48 | USP | Lubricant |
| Core tablet Weight (mg) | 1070.49 | — | USP | — |
| Film Coating Ingredients | | | | |
| Opadry ® II 85F92209-CN | 42.82 | 3.85 | In-house | Film Coating |
| Yellow | | | | agent |
| Purified Water | q.s. | — | USP | Solvent |
| Total tablet weight(mg) | 1113.31 | 100.00 | | |

For example, in some instances, it has been observed that as the drug load increases P_{GP}-3₃,T₁ relative to the amount of polymer carrier, the release rate is slower. However, it has also been observed that the release rate may vary, in at least some instances, from one polymer carrier to the other; for example, it has been observed that the release rate using Soluplus was slower than when comparable copovidone polymer carriers were used. Empirical results to-date still further suggest that differences in release rate occur when mixtures of polymer carriers are used, as well; for example, it has been observed that an equal parts mixture of Soluplus and a copovidone polymer carrier results in a release rate that is slower than the release rate observed when only Soluplus is used. In addition, it has been observed that as the amount of the copovidone polymer carrier increases in this mixture, the more the release rate decreases.

Bioavailability of the solid dispersion produced by melt extrusion may be assessed by dispersibility studies. The release rate of API from the extrudates may measured in samples after dispensing the extrudates in acidic environment (e.g., 0.1N HCl) at about physiological temperature (e.g., 37° C.) to simulate drug release in the gastrointestinal tract in human. Generally, current empirical results to-date have shown that the formulations with higher drug load have slower API release rate. It is also observed that although higher surfactant level leads to faster API release rate, excessive surfactant may affect the manufacturability of the extrudates.

Once cooled to ambient temperature, the melt-extruded solid dispersion can be stored until ready for use or further processing. It has been determined, e.g., by differential scanning calorimetry (DSC), that extrudates manufactured by the process of the present invention generally are monophasic systems in which the risk of phase separation is negligible even for extended storage durations. If desired, the solid dispersion can be milled or ground to form granules suitable for filling into capsules or for molding or compression to form a tablet, with additional excipients as may be conventionally used in such dosage forms, for example those listed hereinabove.

In one embodiment, a milling apparatus (e.g., a Quadra U5 Comil) equipped with screens having various aperture sizes (in the range of 100 to 1000 μm) may be used for milling the extrudates. The distribution of the sizes of the particles produced by the milling is substantially consistent throughout the milling process and is not significantly affected by the batch size (e.g., from about 100 g to about 45 kg) of the extrudates. The milled extrudate can be further blended with filler (e.g., dicalcium phosphate (DCP) or crosarmellose sodium), or lubricant (e.g., sodium stearyl fumarate). Tabletability, characterized by the tensile strength as a function of compaction pressure, can be assessed on a tablet press simulator (e.g., Presster, Metropolitan Computing Corporation). In general, formulations made from clear extrudates typically show better tabletability than those made from cloudy extrudates. Further, the addition of DCP or other fillers and reduction of particle size (as determined by the screen aperture sizes of Comil), as well as an increase in moisture content in the milled extrudate, all improve tabletability.

Bioavailability of the tablets produced from the milled extrudates may be assessed by in vitro dissolution studies using, for example, USP apparatus Type 2 method. It has been observed in preferred embodiments that about 90% to about 100% of the API may be released from 50 mg tablets within 6 to 8 hours. Similar results may be obtained using either phosphate buffer or 0.01 N HCl. Based on empirical results to-date, the dissolution rate appears to be independent of the particle size of the milled extrudates before compression and the moisture content in the tablets, and is only slightly decreased by the addition of DCP. This appears to support an erosion-based drug release mechanism in the tablets, with the dissolution rate apparently being a function of the tablet composition.

The following is a typical (non-limiting) Hot Melt Extrusion Chart Flow.

The table above shows an illustrative embodiment of a process of hot melt extrusion useful to form a solid dispersion product.

A granulation step includes combining API, one or more pharmaceutically acceptable polymers, one or more pharmaceutically acceptable antioxidants, and one or more other optional ingredients and transforming these components into a granular form. For example, a pharmaceutically acceptable polymer may be provided in a stock form, such as pellets, which are then ground and mixed with API. Other ingredients, such as colloidal silicon dioxide, can be included in the granulation step.

The granulated API, polymer(s), antioxidant, and optional ingredient(s) are then blended/sieved at with the addition of the surfactant(s). For example, once the surfactant is suitably blended with the granulated API, polymer(s), antioxidant, and optional ingredient(s) to form an essentially uniform mixture, the mixture can be further sieved to select a uniform size.

At this point, the blended and sieved mixture can be processed by an extrusion and calendering where the mixture is subjected to pressure and/or heat suitable to melt at least a portion of the mixture. For example, a screw-type extruder can be used in this step. Suitable screw-type extruders include a Leistritz Micro 18 or Micro 27 twin-screw extruder.

Following extrusion, the mixture or extrudate can be subjected to calendering by pressing with a roller or a series of rollers. That is the mixture from the blending/sieving step is first extruded and then calendered. Calendering can include subjecting the extrudate to a roller having one or more cavities that press the extrudate into a lentil form or other shape.

The extruded and calendered material is cooled and allowed to return to a substantially solid phase prior to being milled, where the solid is ground, pulverized or broken down into smaller particles. The milled material is then blended and/or sieved following addition of one or more optional ingredients such as Silicon Dioxide, and Sodium Steatyl Fumarate.

A compression forces the blended and/or sieved material into the desired final form, such as a tablet providing a particular dosage of the API. Finally, a coating may be applied to the compressed material. The coating, such as a polymer coating, can make the compressed material smoother and easier to swallow, can control the release rate of the API, can make the material more resistant to the environment (extending shelf life), and/or can enhance the tablet's appearance.

### Part F. Dosage Forms and Regimen

The terms “orally deliverable”, “oral administration” and “orally administered” herein refer to administration to a subject per os (p.o.), that is, administration wherein the composition is immediately swallowed, for example with the aid of a suitable volume of water or other potable liquid. “Oral administration” is distinguished herein from intraoral administration, e.g., sublingual or buccal administration or topical administration to intraoral tissues such as periodontal tissues, that does not involve immediate swallowing of the composition.

The active ingredient form (e.g., parent compound or salt), the polymeric carrier(s), surfactant(s) and other optional ingredients should be selected, and relative amounts of these components should be used, to provide a solid dispersion or dosage form having acceptable bioabsorption when administered orally. Such bioabsorption can be evidenced, for example, by the pharmacokinetic (PK) profile of the solid dispersion or dosage form, more particularly by the Cₘₐₓ or AUC, for example AUCo-24 or AUCo-. at a particular dose or over a range of doses. Illustratively, bioavailability can be expressed as a percentage, for example using the parameter F, which computes AUC for oral delivery of a test composition as a percentage of AUC for intravenous (i.v.) delivery of the drug in a suitable solvent, taking into account any difference between oral and i.v. doses.

Bioavailability can be determined by PK studies in humans or in any suitable model species. For present purposes, a dog model is generally suitable. In various illustrative embodiments, compositions of the invention exhibit oral bioavailability of at least about 15%, at least about 20%, at least about 25% or at least about 30%, up to or exceeding about 50%, in a dog model, when administered as a single dose of about 2.5 to about 10 mg/kg to fasting or non-fasting animals.

Compositions embraced herein, including compositions described generally or with specificity herein, are useful for orally delivering a drug that is a compound of Formula I or a pharmaceutically acceptable salt thereof to a subject. Accordingly, a method of the invention for delivering such a drug to a subject comprises orally administering a composition as described above.

The subject can be human or non-human (e.g., a farm, zoo, work or companion animal, or a laboratory animal used as a model) but in an important embodiment the subject is a human patient in need of the drug, for example to treat a disease characterized by apoptotic dysfunction and/or overexpression of an anti-apoptotic Bcl-2 family protein. A human subject can be male or female and of any age. The patient is typically an adult, but a method of the invention can be useful to treat a childhood cancer such as leukemia, for example acute lymphocytic leukemia, in a pediatric patient.

The composition is normally administered in an amount providing a therapeutically effective daily dose of the drug. The term “daily dose” herein means the amount of drug administered per day, regardless of the frequency of administration. For example, if the subject receives a unit dose of 150 mg twice daily, the daily dose is 300 mg. Use of the term “daily dose” will be understood not to imply that the specified dosage amount is necessarily administered once daily. However, in a particular embodiment the dosing frequency is once daily (q.d.), and the daily dose and unit dose are in this embodiment the same thing.

What constitutes a therapeutically effective dose depends on the particular compound, the subject (including species and body weight of the subject), the disease (e.g., the particular type of cancer) to be treated, the stage and/or severity of the disease, the individual subject's tolerance of the compound, whether the compound is administered in monotherapy or in combination with one or more other drugs, e.g., other chemotherapeutics for treatment of cancer, and other factors. Thus the daily dose can vary within wide margins, for example from about 10 to about 1,000 mg. Greater or lesser daily doses can be appropriate in specific situations. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving appropriate frequency and duration of administration. It is strongly preferred that, while the daily dose selected is sufficient to provide benefit in terms of treating the cancer, it should not be sufficient to provoke an adverse side-effect to an unacceptable or intolerable degree. A suitable therapeutically effective dose can be selected by the physician of ordinary skill without undue experimentation based on the disclosure herein and on art cited herein, taking into account factors such as those mentioned above. The physician may, for example, start a cancer patient on a course of therapy with a relatively low daily dose and titrate the dose upwards over a period of days or weeks, to reduce risk of adverse side-effects.

Illustratively, suitable doses of a compound of Formula A are generally about 25 to about 1,000 mg/day, more typically about 50 to about 800 mg/day or about 200 to about 500 mg/day, for example about 50, about 100, about 150, about 200, about 250, about 300, about 350, about 400, about 450, about 500, about 550, about 600, about 650, about 700, about 750, about 800 mg/day, administered at an average dosage interval of about 3 hours to about 7 days, for example about 8 hours to about 3 days, or about 12 hours to about 2 days. In most cases a once-daily (q.d.) administration regimen is suitable.

An “average dosage interval” herein is defined as a span of time, for example one day or one week, divided by the number of unit doses administered over that span of time. For example, where a drug is administered three times a day, around 8 am, around noon and around 6 pm, the average dosage interval is 8 hours (a 24-hour time span divided by 3). If the drug is formulated as a discrete dosage form such as a tablet or capsule, a plurality (e.g., 2 to about 10) of dosage forms administered at one time is considered a unit dose for the purpose of defining the average dosage interval.

Where the composition is in the form of a capsule, one to a small plurality of capsules can be swallowed whole, typically with the aid of water or other imbibable liquid to help the swallowing process. Suitable capsule shell materials include, without limitation, gelatin (in the form of hard gelatin capsules or soft elastic gelatin capsules), starch, carrageenan and HPMC.

Administration can be with or without food, i.e., in a non-fasting or fasting condition. It is generally preferred to administer the present compositions to a non-fasting patient.

### Part G. Montherapies and Combination Therapies

“Combination therapy” includes the administration of the Compositions of the invention in further combination with other biologically active ingredients (such as, but not limited to, a second and different antineoplastic agent) and non-drug therapies (such as, but not limited to, surgery or radiation treatment). For instance, the Compositions of the invention can be used in combination with other pharmaceutically active compounds, or non-drug therapies, preferably compounds that are able to enhance the effect of the Compositions of the invention. The Compositions of the invention can be administered simultaneously (as a single preparation or separate preparation) or sequentially to the other therapies. In general, a combination therapy envisions administration of two or more drugs/treatments during a single cycle or course of therapy.

In one embodiment, the Compositions of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the Compositions may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMKla, CAMK1b, CAMKld, CAMKlg, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CKlal, CK1d, CKlepsilon, CK1 g1, CK1 g2, CK1 g3, CK2a, CK2a2, c-KIT, CLK1, CLK2, CLK3, CLK4, c-MER, c-MET, COT1/MAP3K8, CSK, c-SRC, CSF1R, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRKIA, DYRKIB, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/IiER2, ERBB4/IER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDR/VEGFR2, KHS/MAP4K5, LATS1, LATS2, LCK, LCK2/ICK, LKB1, LIM K1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEKi11, NIK/MAP3K14, NLK, OSR1/OXSR1, P38a/MAPK14, P38b/MAPK11, P38d/MAPK13, P38 g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKgl, PHKg2, PI3Ka, (p110a/p85a), PI3Kb, (p110b/p85a), PI3Kd, (p110d/p85a), PI3Kg(pl20 g), PIM1, PIM2, PIM3, PKA, PKAcb, PKAcg, PKCa, PKCbl, PKCb2, PKCd, PKCepsilon, PKCeta, PKCg, PKCiota, PKCmu/PRKD1, PKCnu/PRKD3, PKCtheta, PKCzeta, PKD2/PRKD2, PKGla, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TAO1, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEEl, WNK1, WNK2, WNK3, YES/YES1, ZAK/MLTK, ZAP70, ZIPK/DAPK3, KINASE, MUTANTS, ABL1(E255K), ABL1(F317I), ABL1(G250E), ABL1(H396P), ABL1(M351T), ABL1(Q252H), ABL1(T315I), ABL1(Y253F), ALK (C1156Y), ALK(L1196M), ALK (F1 174L), ALK (R1275Q), BRAF(V599E), BTK(E41K), CHK2(I157T), c-Kit(A829P), c-KIT(D816H), c-KIT(D816V), c-Kit(D820E), c-Kit(N822K), C-Kit (T670I), c-Kit(V559D), c-Kit(V559D/V654A), c-Kit(V559D/T670I), C-Kit (V560G), c-KIT(V654A), C-MET(D1228H), C-MET(D1228N), C-MET(F1200I), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (I2020T), LRRK2 (R1441C), p38a(T106M), PDGFRa(D842V), PDGFRa(T674I), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the Compositions of the invenstion may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g. Bcl-xL, BCL-2, Bcl-w), histone deacetylases (HDAC), histone acetyltransferases (HAT), and methyltransferase (e.g., histone lysine methyltransferases, histone arginine methyltransferase, DNA methyltransferase, etc), and other immunotherapies(e.g anti-PD1, anti-PDL1, anti-CTLA4, CAR-T, IDO, A2A antagonist etc).

In another aspect of the invention, the Compositions of the invention are administered in combination with one or more of other anti-cancer agents that include, but are not limited to, gene therapy, RNAi cancer therapy, chemoprotective agents (e.g., amfostine, mesna, and dexrazoxane), antibody conjugate(e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

In another aspect of the invention, the Compositions of the invention are administered in combination with one or more of BiTE antibodies. BiTE antibodies are bi-specific antibodies that direct T-cells to attack cancer cells by simultaneously binding the two cells.

The T-cell then attacks the target cancer cell. Examples of BiTE antibodies include, but are not limited to, adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (Sutton et al. (1997) J. Immunol. 158:5783-5790).

In another aspect of the invention, the Compositions of the invention are administered in combination with one or more of siRNAs, which are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths {e.g., 10-200 bps) and structures {e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand. For example, siRNAs targeting Mcl-1 have been shown to enhance the activity of ABT-263 or ABT-737 in various tumor cell lines (Tse et al. (2008) Cancer Res. 68:3421-3428 and references therein).

In another aspect of the invention, the Compositions of the invention are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the Compositions of the invention are administered in combination with one or more of radiation therapy, surgery, or anti-cancer agents that include, but are not limited to, DNA damaging agents, anti-metabolites, topoisomerase inhibitors, anti-microtubule agents, kinase inhibitors, epigenetic agents, HSP90 inhibitors, PARP inhibitors, and antibodies targeting VEGF, HER2, EGFR, CD50, CD20, CD30, CD33, etc.

In certain embodiments, the Compositions of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, pembrolizumab, nivolumab, atezolizumab, durvalumab, avelumab, as tisagenlecleucel, axicabtagene ciloleucel, radiation therapy, or surgery.

The invention further provides methods for the prevention or treatment of a neoplastic disease or autoimmune disease. In one embodiment, the invention relates to a method of treating a neoplastic disease or autoimmune disease, in a subject in need of treatment comprising administering to said subject a therapeutically effective amount of a compound of the invention. In one embodiment, the invention further provides for the use of a compound of the invention in the manufacture of a medicament for halting or decreasing a neoplastic disease or autoimmune disease.

In certain embodiments, the neoplastic disease is a lung cancer, head and neck cancer, central nervous system cancer, prostate cancer, testicular cancer, colorectal cancer, pancreatic cancer, liver cancer, stomach cancer, biliary tract cancer, esophageal cancer, gastrointestinal stromal tumor, breast cancer, cervical cancer, ovarian cancer, uterine cancer, leukemia, lymphomas, multiple myeloma, melanoma, basal cell carcinoma, squamous cell carcinoma, bladder cancer, renal cancer, sarcoma, mesothelioma, thymoma, myelodysplastic syndrome, or myeloproliferative disease.

The autoimmune diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barre syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

### EXAMPLES

The following examples are merely illustrative, and do not limit this disclosure in any way. For example, it will be appreciate that lab-scale compositions or formulations, or extrusion blends, referenced herein may in general be scaled up in view of the details provided without departing from the intended scope of the present application.

Trademarked ingredients used in the examples, which can be substituted with comparable ingredients from other suppliers, include Tween™ 80 of Uniqema (polysorbate 80 surfactant), and Soluplus™ of BASF (graft copolymer of polyethylene glycol, polyvinyl caprolactam and polyvinyl acetate).

In the Examples, “API” (active pharmaceutical ingredient) can be any compound of Formula A. The following compounds are more specifically contemplated for formulation according to the examples:
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-(trifluoromethyl)-3,4-dihydro-2H-pyrrolo[3′,2′:5,6]pyrido[2,3-b][1,4]oxazepin-1(7H)-yl)benzamide.

### Example 1: Melt Extrusion Feasibility Study

Feasibility experiments were performed to evaluate the influence of different excipients on extrudates with embedded amorphous API. In these experiments, the polymers included Eudragit E100, KOLLIDON® VA64, KOLLIDON® 17 PF, KOLLIDON® 25, Hypromellose with viscosity of 15,000 mPaS (HPMC), and API.

The following table shows that KOLLIDON® VA64 (Copovidone VA64) and Eudragit E100 are suitable polymer for hot melt extrusion.

| Polymer | Melting Conditions | Appearance |
|---|---|---|
| KOLLIDON ® 17 | 5 minute, | Clear yellow glass like, brittle, |
| PF | 200° C. | hard and not sticky |
| KOLLIDON ® 25 | Did not melt | Yellow/black powder, unchanged |
| KOLLIDON ® | 15 minute, | Clear yellow glass like, brittle, |
| VA64 | 160° C. | hard and not sticky |
| Eudragit E100 | 20 minute, | Clear yellow glass like, brittle, |
| | 160° C. | hard and not sticky |
| HPMC | Did not melt | White/black powder |

### Example 2: Tablets Formed by a Process Including Melt-Extrusion

The following components are combined and melt-extruded to form an extrudate:

| Extrude Composition | Weight (%) |
|---|---|
| API | 12 |
| Copovidone VA64 | 80 |
| Tween ™ 80 | 7 |
| Silicon Dioxide | 1 |

In particular, these components of Copovidone VA64, API, Tween™ 80, and Silicon Dioxide can be processed as shown in Table 1 by granulation, blending and/or sieving, extrusion and calendering. The extrusion/calendering is performed using a screw-type extruder:
1. Weight out API, KOLLIDON® VA 64, PB80 and Colloidal SiO₂ in a container according to the above table.
2. Mix components together with a spoon till a uniform paste form.
3. Set hot melt extruder typically at temperature 135-155° C. and rotor speed 72/min.
4. Add 5 g KOLLIDON® VA64 into extruder to prime the extruder and clean out the dirty stuff in the system.
5. Add the powder acquired form Step 2 into extruder to get extruded product.
6. Collect the HIMIE product and weight it.

The resulting extrudate can then be processed as shown in Table 1 by milling, blending/sieving, compression, and coating. The extrudate is further combined with the following ingredients to provide the following tablet formulation:

| Tablet Composition | Weight (%) |
|---|---|
| Extrudate | 78.5 |
| Dicalcium Phosphate | 20 |
| Sodium Stearyl Fumarate | 0.5 |
| Colloidal SiO² | 1 |

Procedure of Tablet Granules
7. Use Comil to mill the HIMIE product at 2,200 rpm and cheese grater screen (US mesh #12).
8. Use pestle to grind bigger particles in a mortar.
9. Collect the shattered product and add dicalcium phosphate, sodium stearyl fumarate and colloidal SiO2 according to the above table.
10. Use jet-mill at 80-90 psi to reduce particle size
11. Collect the fine granules for tablet compression.

Tablet Production
12. Choose the right tool set of punches and dies for one-gram tablet.
13. Weight 1.1 g fine granules and transfer it into tablet compression machine.
14. Collet the final tablet and weight again to make sure if the weight is within the range of 1.05 and 1.15.
15. Finally coat the tablets using OPADRY® II Yellow (85F92209CN)

Dissolution Test (Tablet): The dissolution medium is the Simulated gastric fluid (SGF) SGF with 1% w/w PB80 and the 500 mL dissolution medium was used for for each tablet. The following table shows the result dissolution test. F5 with the disintegrant (crospovidone) in SGF with 1% w/w PB80 has instant release within 15 min and it can almost reach 100%. F4 without the disintegrant (crospovidone) in SGF with 1% w/w PB80 also has fast release rate to 70% in 30 min. F1 and F2 (with KOLLIDON® VA64 as the matrix) without disintegrant and have slow release rate about 20% in 120 minutes.

| Time (min) | Tablet F3 |
|---|---|
| 0 | 0 |
| 5 | 0.4% |
| 10 | 1.1% |
| 15 | 2.1% |
| 30 | 5.1% |
| 60 | 10.6% |
| 120 | 22.8% |

To investigate phase separation potentials, differential scanning calorimetry (DSC) cycle tests were performed on extrudate. The extrudate was milled using a standard laboratory ball mill, weighted into closed DSC crucibles and placed into the instrument (Mettler Toledo DSC 1/700/183). The samples were heated from 60° C. to 100° C. following a cooling to 60° C. (1 Cycle). This procedure was repeated up to 5 times (5 Cycles). The results indicated that extrudate was a monophasic system after manufacture and phase separation was not likely to happen when the formulation was stored at long term and accelerated conditions below or even close to its glass transition temperature.

Dog PK study of the tablets (100 mg/tablet): The pharmacokinetics of tablet were evaluated in beagle dog via Oral Administration. The oral doses were administered by gavage. The PK time point for PO arm was 15, 30 min, 1, 2, 4, 6, 8, 12, 24 hours post dose. Approximately 1.5 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at-75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: Co, CL, Va, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary. The dog PK is shown below. The results indicate that this tablet shows satisfying pharmacokinetic profile.

| | T½(h) | Tₘₐₓ(h) | Cₘₐₓ(ng/mL) | AUC₍₀₋ₜ₎(h*ng/ml) |
|---|---|---|---|---|
| Tablet F3 | 12.6 | 4.25 | 4,090 | 54,280 |

### Example 3: The Hot Melt-Extrusion and Tablet Below are Prepared by Methods Substantially Identical, Similar, or Analogous to Those Disclosed in Examples 1 and 2

| Extrude Composition | Weight (%) |
|---|---|
| API | 10 |
| Copovidone VA64 | 82 |
| Tocophersolan (TPGS) | 7 |
| Silicon Dioxide | 1 |

| Tablet Composition | Weight (%) |
|---|---|
| Extrudate | 78.5% |
| Dicalcium Phosphate | 20% |
| Sodium Stearyl Fumarate | 0.5% |
| Colloidal SiO₂ | 1% |

### Example 4: The Hot Melt-Extrusion and Tablet Below are Prepared by Methods Substantially Identical, Similar, or Analogous to Those Disclosed in Examples 1 and 2

| Extrude Composition | Weight (%) |
|---|---|
| API | 12 |
| Copovidone VA64 | 78 |
| Tocophersolan (TPGS) | 7 |
| Lauroglycol 90 | 2 |
| Silicon Dioxide | 1 |

| Tablet Composition | Weight (%) |
|---|---|
| Extrudate | 78.5% |
| AVICEL | 14.5% |
| Crospovidone | 6% |
| Colloidal SiO₂ | 1% |

### Example 5: The Hot Melt-Extrusion and Tablet Below are Prepared by Methods Substantially Identical, Similar, or Analogous to Those Disclosed in Examples 1 and 2

| Extrude Composition | Weight (%) |
|---|---|
| API | 10 |
| Eudragit E100 | 82 |
| Tween ™ 80 | 7 |
| Silicon Dioxide | 1 |

| Tablet Composition | Weight (%) |
|---|---|
| Extrudate | 73.8% |
| Dicalcium Phosphate | 18.8% |
| Sodium Stearyl Fumarate | 0.47% |
| Crospovidone | 6% |
| Colloidal SiO₂ | 0.94% |

### Example 6: The Hot Melt-Extrusion and Tablet Below are Prepared by Methods Substantially Identical, Similar, or Analogous to Those Disclosed in Examples 1 and 2

| Extrude Composition | Weight (%) |
|---|---|
| API | 12 |
| Copovidone VA64 | 81 |
| Tween ™ 80 | 5 |
| Silicon Dioxide | 1 |

| Tablet Composition | Weight (%) |
|---|---|
| Extrudate | 78.5% |
| Dicalcium Phosphate | 20% |
| Sodium Stearyl Fumarate | 0.5% |
| Colloidal SiO₂ | 1% |

### Example 7: The Hot Melt-Extrusion and Tablet Below are Prepared by Methods Substantially Identical, Similar, or Analogous to Those Disclosed in Examples 1 and 2

| Extrude Composition | Weight (%) |
|---|---|
| API | 18 |
| Copovidone VA64 | 74 |
| Tween ™ 80 | 7 |
| Silicon Dioxide | 1 |

| Tablet Composition | Weight (%) |
|---|---|
| Extrudate | 78.5% |
| Dicalcium Phosphate | 20% |
| Sodium Stearyl Fumarate | 0.5% |
| Colloidal SiO₂ | 1% |

### Example 8: The Hot Melt-Extrusion and Tablet Below are Prepared by Methods Substantially Identical, Similar, or Analogous to Those Disclosed in Examples 1 and 2

During the manufacture of the Drug Product (tablet), it is always critical to control the amount of any impurities below 0.2% formed during the hot met extrusion process. The following table shows the impurity profile of API, and the solid disperse without and with antioxidant:

| | Extrude Without | Extrude With antioxidant |
|---|---|---|
| Composition | antioxidant weight (%) | weight (%) |
| API | 12 | 11.9 |
| Copovidone VA64 | 81 | 79.2 |
| Tween ™ 80 | 5 | 6.9 |
| Sodium Ascorbate | | 1 |
| Silicon Dioxide | 1 | 1 |

| | | Extrude | Extrude |
|---|---|---|---|
| | API | Without antioxidant | With antioxidant |
| RRT 0.77 min | 0.06 | 0.07 | 0.04 |
| RRT 0.87 min | 0.09 | 0.06 | 0.13 |
| RRT 0.94 min | 0.14 | 0.15 | 0.11 |
| RRT 0.97 min | 0.21 | 0.12 | 0.10 |
| RRT 1.05 min | | 0.43 | |
| RRT 1.12 min | 0.08 | 0.42 | 0.05 |
| RRT 1.17 min | 0.40 | 0.44 | 0.4 |
| RRT 1.19 min | | | |
| RRT 1.54 min | 0.1 | | |
| Total impurity % | 1.30 | 2.86 | 1.45 |

RRT in the tablet means the PLC retention time. Based on our internal force degradation study, we have know that the impurities at RRT 1.05 and 1.12 are formed by oxidation. The results in the table shows that adding antioxidant during the hot melt extrusion can significantly reduce the oxidative impurity thus the total impurity %.

### Example 9: The Hot Melt-Extrusion and Tablet Below are Prepared by Methods Substantially Identical, Similar, or Analogous to Those Disclosed in Examples 1 and 2

| Extrude Composition | Weight (%) |
|---|---|
| API | 11.9 |
| Copovidone VA64 | 79.2 |
| Tween ™ 80 | 6.9 |
| Sodium Ascorbate | 1 |
| Silicon Dioxide | 1 |

| Tablet Composition | Weight (%) |
|---|---|
| Extrudate | 78.5% |
| Dicalcium Phosphate | 20.0% |
| Sodium Stearyl Fumarate | 0.5% |
| Colloidal SiO₂ | 1% |

### Example 10: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API^{[2]} | 11.89% |
| Copovidone VA64 | 79.23% |
| Tween ™ 80 | 6.93% |
| VC | 0.95% |
| Silicon Dioxide | 1.00% |

^{[1]}: In this study, the HME temprature is 150° C.
^{[2]}: In this study, the compound is Compound 2-9.

| Tablet Composition | Weight (%) |
|---|---|
| Extrudate | 78.59% |
| Croscarmellose Sodium | 19.91% |
| Sodium Stearyl Fumarate | 0.50% |
| Colloidal SiO₂ | 1.00% |

### Example 11: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 12.00% |
| Copovidone VA64 | 79.00% |
| Tween ™ 80 | 7.00% |
| VC | 1.00% |
| Silicon Dioxide | 1.00% |

| Tablet Composition | Weight (%) |
|---|---|
| Extrudate | 79.25% |
| Dicalcium Phosphate | 19.25% |
| Sodium Stearyl Fumarate | 0.50% |
| Colloidal SiO₂ | 1.00% |

^{[1]}: In this study, the HME temperature is 150° C.

### Example 12: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 14.00% |
| Copovidone VA64 | 78.00% |
| Tween ™ 80 | 7.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temperature is 150° C.

### Example 13: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 14.00% |
| Copovidone VA64 | 77.00% |
| Tween ™ 80 | 8.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO2 | 1.00% |

^{[1]}: In this study, the HME temperature is 150° C.

### Example 14: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 14.00% |
| Copovidone VA64 | 76.00% |
| Tween ™ 80 | 9.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temperature is 150° C.

### Example 15: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 16.00% |
| Copovidone VA64 | 76.00% |
| Tween ™ 80 | 7.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temperature is 150° C.

### Example 16: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 16.00% |
| Copovidone VA64 | 75.00% |
| Tween ™ 80 | 8.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temperature is 150° C.

### Example 17: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 16.00% |
| Copovidone VA64 | 74.00% |
| Tween ™ 80 | 9.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temperature is 150° C.

### Example 18: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 18.00% |
| Copovidone VA64 | 74.00% |
| Tween ™ 80 | 7.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 19: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 18.00% |
| Copovidone VA64 | 73.00% |
| Tween ™ 80 | 8.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 20: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 18.00% |
| Copovidone VA64 | 72.00% |
| Tween ™ 80 | 9.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 21: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 72.00% |
| Tween ™ 80 | 7.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 22: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 71.00% |
| Tween ™ 80 | 8.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 23: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 70.00% |
| Tween ™ 80 | 9.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 24: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 22.50% |
| Copovidone VA64 | 69.50% |
| Tween ™ 80 | 7.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 25: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| API | 25.00% |
| Copovidone VA64 | 67.00% |
| Tween ™ 80 | 7.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 150° C.

### Example 26: The Hot Melt-Extrusion^{[1]} and Tablet Below are Prepared by Methods Substantially Identical, Similar, or Analogous to Those Disclosed in Examples 1 and 2

| Extrude Composition | Weight (%) |
|---|---|
| APJ^{[2]} | 15.00% |
| Copovidone VA64 | 75.00% |
| Tween ™ 80 | 9.00% |
| Silicon Dioxide | 1.00% |

^{[1]}: In this study, the HME temperature is 145° C.
^{[2]}: In this study, the compound is Compound 3-8.

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

### Example 27: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 12.00% |
| Copovidone VA64 | 79.00% |
| Tween ™ 80 | 8.00% |
| Silicon Dioxide | 1.00% |

^{[1]}: In this study, the HME temperature is 135° C.

### Example 28: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 76.00% |
| Tween ™ 80 | 8.00% |
| Silicon Dioxide | 1.00% |

^{[1]}: In this study, the HME temperature is 135° C.

### Example 29: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 71.00% |
| Tween ™ 80 | 8.00% |
| Silicon Dioxide | 1.00% |

^{[1]}: In this study, the HME temperature is 135° C.

### Example 30: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 25.00% |
| Copovidone VA64 | 66.00% |
| Tween ™ 80 | 8.00% |
| Silicon Dioxide | 1.00% |

^{[1]}: In this study, the HME temperature is 135° C.

### Example 31: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| Kolliphor HS15 | 9.00% |
| Silicon Dioxide | 1.00% |

^{[1]}: In this study, the HME temperature is 140° C.

### Example 32: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| Kolliphor RH40 | 9.00% |
| Silicon Dioxide | 1.00% |

^{[1]}: In this study, the HME temperature is 140° C.

### Example 33: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| LABRAFIL M 1944CS | 9.00% |
| Silicon Dioxide | 1.00% |

^{[1]}: In this study, the HME temperature is 150° C.

### Example 34: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| Transcutol HP | 9.00% |
| Silicon Dioxide | 1.00% |

^{[1]}: In this study, the HME temperature is 130° C.

### Example 35: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| LABRASOL ALF | 9.00% |
| Silicon Dioxide | 1.00% |

^{[1]}: In this study, the HME temperature is 130° C.

### Example 36: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| Kolliphor ELP | 9.00% |
| Silicon Dioxide | 1.00% |

^{[1]}: In this study, the HME temperature is 135° C.

### Example 37: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 79.00% |
| Transcutol HP | 5.00% |
| Silicon Dioxide | 1.00% |

^{[1]}: In this study, the HME temperature is 120° C.

### Example 38: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 69.00% |
| Transcutol HP | 15.00% |
| Silicon Dioxide | 1.00% |

^{[1]}: In this study, the HME temperature is 80° C.

### Example 39: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 79.00% |
| Kolliphor ELP | 5.00% |
| Silicon Dioxide | 1.00% |

^{[1]}: In this study, the HME temprature is 130° C.

### Example 40: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 64.00% |
| Kolliphor ELP | 20.00% |
| Silicon Dioxide | 1.00% |

^{[1]}: In this study, the HME temprature is 130° C.

### Example 41: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 81.00% |
| LABRASOL ALF | 3.00% |
| Silicon Dioxide | 1.00% |

^{[1]}: In this study, the HME temprature is 140° C.

### Example 42: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 79.00% |
| LABRASOL ALF | 5.00% |
| Silicon Dioxide | 1.00% |

^{[1]}: In this study, the HME temprature is 130° C.

### Example 43: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 78.00% |
| LABRASOL ALF | 6.00% |
| Silicon Dioxide | 1.00% |

^{[1]}: In this study, the HME temprature is 130° C.

### Example 44: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 77.00% |
| LABRASOL ALF | 7.00% |
| Silicon Dioxide | 1.00% |

^{[1]}: In this study, the HME temprature is 130° C.

### Example 45: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 76.00% |
| LABRASOL ALF | 8.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 130° C.

### Example 46: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 71.00% |
| LABRASOL ALF | 8.00% |
| Silicon Dioxide | 1.00% |

^{[1]}: In this study, the HME temprature is 130° C.

### Example 47: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 25.00% |
| Copovidone VA64 | 66.00% |
| LABRASOL ALF | 8.00% |
| Silicon Dioxide | 1.00% |

^{[1]}: In this study, the HME temprature is 130° C.

### Example 48: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| LABRASOL ALF | 9.00% |
| Silicon Dioxide | 1.00% |

^{[1]}: In this study, the HME temprature is 130° C.

### Example 49: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 64.00% |
| LABRASOL ALF | 20.00% |
| Silicon Dioxide | 1.00% |

^{[1]}: In this study, the HME temprature is 80° C.

### Example 50: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 69.00% |
| LABRASOL ALF | 10.00% |
| Silicon Dioxide | 1.00% |

^{[1]}: In this study, the HME temprature is 130° C.

### Example 51: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 67.00% |
| LABRASOL ALF | 12.00% |
| Silicon Dioxide | 1.00% |

^{[1]}: In this study, the HME temprature is 130° C.

### Example 52: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 25.00% |
| Copovidone VA64 | 64.00% |
| LABRASOL ALF | 10.00% |
| Silicon Dioxide | 1.00% |

^{[1]}: In this study, the HME temprature is 130° C.

### Example 53: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 25.00% |
| Copovidone VA64 | 61.50% |
| LABRASOL ALF | 12.50% |
| Silicon Dioxide | 1.00% |

^{[1]}: In this study, the HME temprature is 130° C.

### Example 54: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 25.00% |
| Copovidone VA64 | 59.00% |
| LABRASOL ALF | 15.00% |
| Silicon Dioxide | 1.00% |

^{[1]}: In this study, the HME temprature is 130° C.

### Example 55: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 81.00% |
| Gelucire 44/14 | 3.00% |
| Silicon Dioxide | 1.00% |

^{[1]}: In this study, the HME temprature is 140° C.

### Example 56: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 78.00% |
| Gelucire 44/14 | 6.00% |
| Silicon Dioxide | 1.00% |

^{[1]}: In this study, the HME temprature is 140° C.

### Example 57: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 77.00% |
| Gelucire 44/14 | 7.00% |
| Silicon Dioxide | 1.00% |

| Tablet Composition | Weight (%) |
|---|---|
| Extrudate | 78.50% |
| Dicalcium Phosphate | 20.00% |
| Sodium Stearyl Fumarate | 0.50% |
| Colloidal SiO₂ | 1.00% |

^{[1]}: In this study, the HME temprature is 140° C.

### Example 58: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 20.00% |
| Copovidone VA64 | 72.00% |
| Gelucire 44/14 | 7.00% |
| Silicon Dioxide | 1.00% |

^{[1]}: In this study, the HME temprature is 140° C.

### Example 59: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 25.00% |
| Copovidone VA64 | 67.00% |
| Gelucire 44/14 | 7.00% |
| Silicon Dioxide | 1.00% |

^{[1]}: In this study, the HME temprature is 140° C.

### Example 60: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 76.00% |
| Gelucire 44/14 | 8.00% |
| Silicon Dioxide | 1.00% |

^{[1]}: In this study, the HME temprature is 135° C.

### Example 61: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 75.00% |
| Gelucire 44/14 | 9.00% |
| Silicon Dioxide | 1.00% |

^{[1]}: In this study, the HME temprature is 135° C.

### Example 62: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 74.00% |
| Gelucire 44/14 | 10.00% |
| Silicon Dioxide | 1.00% |

^{[1]}: In this study, the HME temprature is 135° C.

### Example 63: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 81.00% |
| Gelucire 50/13 | 3.00% |
| Silicon Dioxide | 1.00% |

^{[1]}: In this study, the HME temprature is 155° C.

### Example 64: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 78.00% |
| Gelucire 50/13 | 6.00% |
| Silicon Dioxide | 1.00% |

^{[1]}: In this study, the HME temprature is 155° C.

### Example 65: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 76.00% |
| Gelucire 50/13 | 8.00% |
| Silicon Dioxide | 1.00% |

^{[1]}: In this study, the HME temprature is 155° C.

### Example 66: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| LABRASOL ALF | 8.00% |
| Tween ™ 80 | 4.00% |
| Silicon Dioxide | 1.00% |

^{[1]}: In this study, the HME temprature is 130° C.

### Example 67: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| LABRASOL ALF | 8.00% |
| SDS | 4.00% |
| Silicon Dioxide | 1.00% |

^{[1]}: In this study, the HME temprature is 130° C.

### Example 68: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| LABRASOL ALF | 8.00% |
| PEG400 | 4.00% |
| Silicon Dioxide | 1.00% |

^{[1]}: In this study, the HME temprature is 130° C.

### Example 69: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| LABRASOL ALF | 8.00% |
| PVP K17PF | 4.00% |
| Silicon Dioxide | 1.00% |

^{[1]}: In this study, the HME temprature is 130° C.

### Example 70: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| LABRASOL ALF | 8.00% |
| HPMC VLV | 4.00% |
| Silicon Dioxide | 1.00% |

^{[1]}: In this study, the HME temprature is 130° C.

### Example 71: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| Tween ™ 80 | 8.00% |
| LABRASOL ALF | 4.00% |
| Silicon Dioxide | 1.00% |

^{[1]}: In this study, the HME temprature is 140° C.

### Example 72: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| Tween ™ 80 | 8.00% |
| SDS | 4.00% |
| Silicon Dioxide | 1.00% |

^{[1]}: In this study, the HME temprature is 140° C.

### Example 73: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| Tween ™ 80 | 8.00% |
| PEG400 | 4.00% |
| Silicon Dioxide | 1.00% |

^{[1]}: In this study, the HME temprature is 140° C.

### Example 74: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| Tween ™ 80 | 8.00% |
| PVP K17PF | 4.00% |
| Silicon Dioxide | 1.00% |

^{[1]}: In this study, the HME temprature is 140° C.

### Example 75: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 72.00% |
| Tween ™ 80 | 8.00% |
| HPMC VLV | 4.00% |
| Silicon Dioxide | 1.00% |

^{[1]}: In this study, the HME temprature is 140° C.

### Example 76: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 69.00% |
| LABRASOL ALF | 8.00% |
| SDS | 4.00% |
| Tween ™ 80 | 3.00% |
| Silicon Dioxide | 1.00% |

^{[1]}: In this study, the HME temprature is 130° C.

### Example 77: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 69.00% |
| LABRASOL ALF | 8.00% |
| SDS | 4.00% |
| Gelucire44/14 | 3.00% |
| Silicon Dioxide | 1.00% |

^{[1]}: In this study, the HME temprature is 130° C.

### Example 78: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 69.00% |
| LABRASOL ALF | 8.00% |
| SDS | 4.00% |
| TPGS | 3.00% |
| Silicon Dioxide | 1.00% |

^{[1]}: In this study, the HME temprature is 130° C.

### Example 79: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 69.00% |
| LABRASOL ALF | 8.00% |
| SDS | 4.00% |
| Kollophor HS15 | 3.00% |
| Silicon Dioxide | 1.00% |

^{[1]}: In this study, the HME temprature is 130° C.

### Example 80: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 69.00% |
| LABRASOL ALF | 8.00% |
| SDS | 4.00% |
| Transcutol HP | 3.00% |
| Silicon Dioxide | 1.00% |

^{[1]}: In this study, the HME temprature is 130° C.

### Example 81: The Hot Melt-Extrusion^{[1]} Below are Prepared by Methods Substantially Identical, Similar, or Analogous to Those Disclosed in Examples 1 and 2

| Extrude Composition | Weight (%) |
|---|---|
| API | 15.00% |
| Copovidone VA64 | 69.00% |
| LABRASOL ALF | 8.00% |
| SDS | 4.00% |
| Kollophor P188 | 3.00% |
| Silicon Dioxide | 1.00% |

^{[1]}: In this study, the HME temprature is 130° C.

The data above collectively demonstrate that hot-melt extrusion (HME) formulations prepared according to Examples 7-24 and 26-29, are superior, in that they achieved desired PK profiles in animal model (e.g., Beagle dog having an average weight of about 5-10 kg), including desired AUC₍ₒ₋ₜ₎ value of at least about 30,000 h*ng/ml, at least about 40,000 h*ng/ml, or at least about 50,000 h*ng/ml; and/or reduced oxidative impurity (e.g., less than 80%, 70%, 60%, 55%, 50% of the control level) when antioxidants such as Vitamin C (VC)/ascorbic acid was present in the formulations.

Interestingly, some of the formulations tested exhibited relatively high solubility as evidenced by dissolution profile, yet produced undesirable PK profiles such as low AUC values.

As various changes could be made in the above apparatus and methods without departing from the scope of the disclosure, it is intended that all matter contained in the above description and shown in the accompanying figures shall be interpreted as illustrative and not in a limiting sense.

1. A solid dispersion comprising a compound of Formula A, 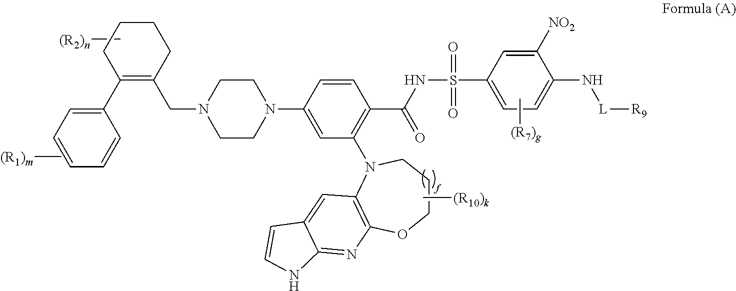
wherein
each of R₁, R₂, R₇, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c},-alkyl-P(O)R_{b}R_{c}, —S(O)(═N(R_{b}))Rₑ, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more R_{d};
Rₐ, R_{b}, Rₑ, R_{bb}, Rec, and R_{d}, independently, is H, D, alkyl, spiroalkyl, alkenyl, alkynyl, alkoxy, alkoxyalkyl, haloalkyl, hydroxyalkyl, aminoalkyl, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;
Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
Z₁ is a bond, (CH₂)p, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;
L is-L₁-L₂-;
L₁ is a bond, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally substituted with one or more R_{d};
L₂ is a bond, or an alkyl in which one or more -Lᵢ-are optionally inserted between any two adjacent carbon atoms;
-Lᵢ-is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;
two of R₁ group, taken together with the atoms to which they are attached, may optionally form a cycloalkyl or heterocycloalkyl, in which said cycloalkyl or heterocycloalkyl of R₁, is optionally substituted with one or more R_{d};
two of R₂ group, taken together with the atom(s) to which they are attached, may optionally form a cycloalkyl or heterocycloalkyl, in which said cycloalkyl or heterocycloalkyl of R₂, is optionally substituted with one or more R_{d};
two of R₇ group, taken together with the atoms to which they are attached, may optionally form a cycloalkyl or heterocycloalkyl, in which said cycloalkyl or heterocycloalkyl of R₇, is optionally substituted with one or more R_{d};
two of R₁₀ group, taken together with the atom(s) to which they are attached, may optionally form a cycloalkyl or heterocycloalkyl, in which said cycloalkyl or heterocycloalkyl of R₁₀, is optionally substituted with one or more R_{d};
R₇ and L group, taken together with the atom to which they are attached, may optionally form a cycloalkyl, or heterocycloalkyl, in which said cycloalkyl or heterocycloalkyl of R₇ and L, is optionally substituted with one or more Rₑ;
R_{b} and R, group, taken together with the atom to which they are attached, may optionally form a cycloalkyl, or heterocycloalkyl, in which said cycloalkyl or heterocycloalkyl of R_{b} and R_{c}, is optionally substituted with one or more Rₑ;
two of R_{d} group, taken together with the atom(s) to which they are attached, may optionally form a cycloalkyl, or heterocycloalkyl, in which said cycloalkyl or heterocycloalkyl of R_{d}, is optionally substituted with one or more Rₑ;
two of Rₑ group, taken together with the atom(s) to which they are attached, may optionally form a cycloalkyl or heterocycloalkyl, in which said cycloalkyl or heterocycloalkyl of Rₑ is optionally substituted with one or more groups selected from H, D, alkyl, alkenyl, alkynyl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, aryl, or heteroaryl;
each of k, g, m, n, p and q is, independently, 0, 1, 2, 3, 4, or 5; and
f is 0 or 1,
or a pharmaceutically acceptable salt thereof; dispersed in a solid matrix that comprises (a) at least one pharmaceutically acceptable water-soluble polymeric carrier, (b) at least one pharmaceutically acceptable surfactant, and optionally, (c) at least one pharmaceutically acceptable antioxidant.

2. The solid dispersion of claim 1, wherein the compound is represented by Formula (A-1), (A-2), or (A-3):

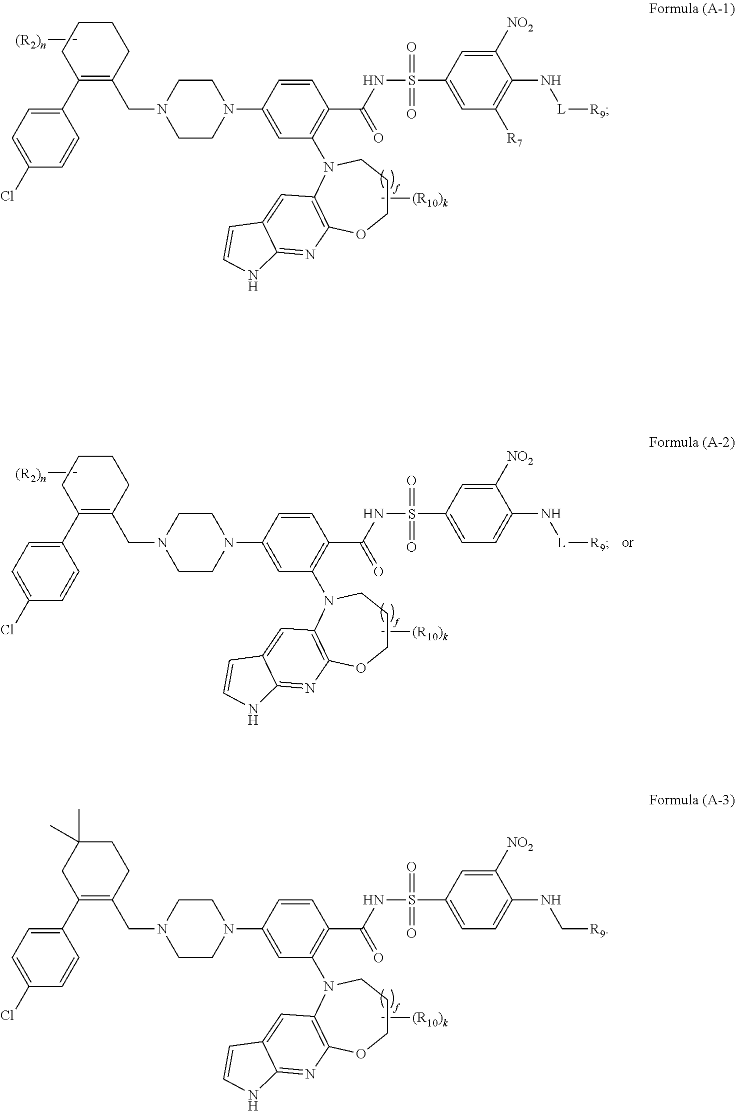

3. (canceled)

4. (canceled)

5. The solid dispersion of claim 1, wherein the compound is selected from the group consisting of
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide, (S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide, (R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-(trifluoromethyl)-3,4-dihydro-2H-pyrrolo[3′,2′:5,6]pyrido[2,3-b][1,4]oxazepin-1(7H)-yl)benzamide.

6. The solid dispersion of claim 5, wherein the compound or salt is present in a parent-compound-equivalent amount of about 5% to about 40% by weight.

7. The solid dispersion of claim 1, wherein i) the at least one polymeric carrier comprises homopolymers and copolymers of N-vinyl lactams, cellulose esters, cellulose ethers, high molecular weight polyalkylene oxides, polyacrylates, polymethacrylates, polyacrylamides, vinyl acetate polymers, graft copolymers of polyethylene glycol, polyvinyl caprolactam and polyvinyl acetate, oligo- and polysaccharides, and/or mixtures thereof; or
ii) the at least one polymeric carrier comprises povidones, copovidones (such as KOLLIDON® VA64 type copovidone), HPMCs, polyethylene glycol/polyvinyl caprolactam/polyvinyl acetate graft copolymers, and/or mixtures thereof; optionally, said at least one polymeric carrier comprises, consists essentially of, or consists of KOLLIDON®

8. (canceled)

9. The solid dispersion of claim 7, wherein
i) the at least one surfactant comprises a non-ionic surfactant,
ii) the at least one surfactant is a non-ionic surfactant; or
iii) the at least one surfactant comprises polyoxyethylene glycerides, fatty acid monoesters of sorbitan, polysorbates (such as TWEEN® 80 brand Polysorbate 80 or Polyoxyethylene (20) sorbitan monooleate), a-tocopheryl polyethylene glycol succinate (TPGS) and/or mixtures thereof.

10. (canceled)

11. (canceled)

12. The solid dispersion of claim 1, wherein
i)said solid dispersion comprises the at least one antioxidant, and wherein said at least one antioxidant comprise ascorbic acid, an ascorbate, a bisulfite, a metabisulfate, a sulfite, curcumin, curcumin derivatives, ursolic acid, resveratrol, resveratrol derivatives, alpha-lipoic acid, thioglycerol, a polyphenol, catachins, grapeseed extract, green tea extract, citric acid, methionine, cysteine, glutathione, tocopherol, propyl gallate, sodium mercaptoacetate, sodium formaldehyde sulfoxylate, ascorbyl palmitate, butylated hydroxyanisole, butylated hydroxytoluene, lecithin, vitamin E, uric acid, and/or mixtures thereof,
ii) the at least one antioxidant comprises, consists essentially of, or consists of ascorbic acid or ascorbate;
iii) the at least one antioxidant is ascorbic acid or ascorbate;
iv) the solid dispersion further comprises at least one glidant: or
v) the solid dispersion further comprises at least one glidant and the at least one glidant comprises colloidal silicon dioxide.

13-16. (canceled)

17. The solid dispersion of claim 1, wherein
i) the compound or salt is present in a parent-compound-equivalent amount of about 5% to about 40% by weight, the at least one polymeric carrier is present in an amount of about 40% to about 85% by weight, the at least one surfactant is present in an amount of about 2.5% to about 20% by weight, and the at least one antioxidant is present in an amount of about 0.25% to about 5% by weight; or
ii) the compound or salt is present in a parent-compound-equivalent amount of about 5% to about 25% (e.g., about 1²-20%, about 15-20%, or about 18%) by weight, the at least one polymeric carrier is present in an amount of about 50% to about 80% (e.g., about 60-80%, or about 70-80%) by weight, the at least one surfactant is present in an amount of about 2.5% to about 15% (e.g., about 5-10%, or about 7-9%) by weight, and the at least one antioxidant is present in an amount of about 0.5% to about 2.5% (e.g., about 0.5-2%, or about 0.5-1%) by weight.

18. (canceled)

19. The solid dispersion of claim 1, further comprising at least one disintegrant (such as 10-30 wt % Croscarmellose Sodium), at least one lubricant (such as 0.2-1.0 wt % Sodium Stearyl Fumarate), and/or at least one coating (such as 2-5 wt % Opadry® II 85F92209-CN Yellow).

20. The solid dispersion of claim 17, wherein the compound is
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,6]pyrido[2,3-b][1,4]oxazin-1(6H)-yl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-(trifluoromethyl)-3,4-dihydro-2H-pyrrolo[3′,2′:5,6]pyrido[2,3-b][1,4]oxazepin-1(7H)-yl)benzamide, or
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-(trifluoromethyl)-3,4-dihydro-2H-pyrrolo[3′,2′:5,6]pyrido[2,3-b][1,4]oxazepin-1(7H)-yl)benzamide.

21. The solid dispersion of claim 20, wherein
i) the at least one polymeric carrier is a copovidone or a vinylpyrrolidone-vinyl acetate copolymer (such as KOLLIDON® VA64 type copovidone),
ii) the at least one surfactant is a polysorbate (such as Polysorbate 80 type surfactant);
iii) the at least one antioxidant is ascorbic acid or sodium ascorbate:
iv) the solid dispersion further comprises at least one glidant; and/or
v) the solid dispersion further comprises at least one glidant and the at least one glidant comprises colloidal silicon dioxide.

22-25. (canceled)

26. The solid dispersion of any one of claim 1, wherein
i) the solid dispersion is a hot-melt-extrusion (HME) formulation,
ii) the solid dispersion comprises any one of the formulations of Examples 7-24 and 26-29, and wherein the API is any one of the compound of claim 5; or
iii) the solid dispersion exhibits an AUC₍ₒ₋ₜ₎ value of at least about 25,000-150,000 h*ng/ml, at least about 30,000-100,000 h*ng/ml, at least about 40,000-80,000 h*ng/ml, or at least about 50,000-60,000 h*ng/ml when a 100 mg oral dose of said solid dispersion is administered by gavage to a 5-10 kg Beagle dog.

27. (canceled)

28. (canceled)

29. A process for preparing a solid dispersion of claim 1, comprising:
(a) subjecting to elevated temperature
(i) an active pharmaceutical ingredient (API) that comprises a compound of Formula (A) or a pharmaceutically acceptable salt thereof,
(ii) a pharmaceutically acceptable water-soluble polymeric carrier,
(iii) a pharmaceutically acceptable surfactant; and
(iv) optionally a pharmaceutically acceptable antioxidant, to create a semi-solid mixture, 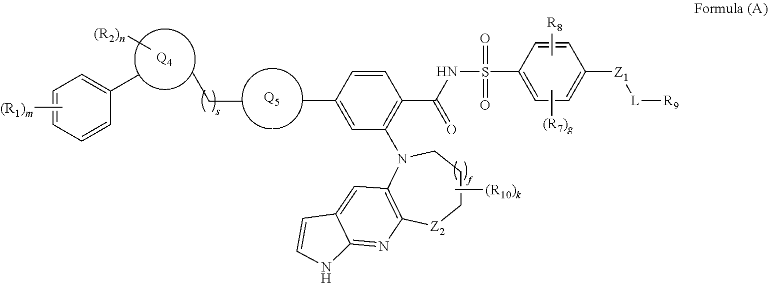
wherein
Q₄ is cycloalkyl, cycloalkenyl, heterocycloalkyl, heterocycloalkenyl, aryl, heteroaryl, or spiro heterocyclic;
Q₅ is cycloalkyl, cycloalkenyl, heterocycloalkyl, heterocycloalkenyl, aryl, heteroaryl, or spiro heterocyclic;
each of R₁, R₂, R₇, R₈, R₉, and R₁₀, independently, is H, D, alkyl, spiroalkyl, alkenyl, alkynyl, cycloalkyl, cycloalkenyl, heterocycloalkyl, spiroheterocycloalkyl, heterocycloalkenyl, aryl, heteroaryl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c},-alkyl-P(O)R_{b}R_{c}, —S(O)(═N(R_{b}))Rₑ, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, aryl, heteroaryl is optionally substituted with one or more R_{d};
Rₐ, R_{b}, Rₑ, R_{bb}, Rec, and R_{d}, independently, is H, D, alkyl, spiroalkyl, alkenyl, alkynyl, alkoxy, alkoxyalkyl, haloalkyl, hydroxyalkyl, aminoalkyl, cycloalkyl, cycloalkenyl, heterocycloalkyl, spiroheterocycloalkyl, heterocycloalkenyl, aryl, or heteroaryl, in which said alkyl, cycloalkyl, cycloalkenyl, heterocycloalkyl, heterocycloalkenyl, aryl, heteroaryl is optionally substituted with one or more Rₑ;
Rₑ, independently, is H, D, alkyl, spiroalkyl, alkenyl, alkynyl, halo, cyano, amine, nitro, hydroxy, ═O, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, spiroheterocycloalkyl, heterocycloalkenyl, aryl, or heteroaryl;
Z₁ is a bond, (CH₂)p, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, OC(O)N(H)(CH₂)ₚ₊₁N(H)(CH₂)_{q}, a bivalent alkenyl group, or a bivalent alkynyl group;
L is-L₁-L₂-;
L₁ is a bond, alkenyl, alkynyl, cycloalkyl, cycloalkenyl, heterocycloalkyl, heterocycloalkenyl, aryl, or heteroaryl, in which said alkenyl, alkynyl, cycloalkyl, cycloalkenyl, heterocycloalkyl, heterocycloalkenyl, aryl, or heteroaryl is optionally substituted with one or more R_{d};
L₂ is a bond, or an alkyl in which one or more -Lᵢ-are optionally inserted between any two adjacent carbon atoms;
-L₁-is —N(Rₐ)—, —O—, —S—, —C(O)—, —S(O₂)—, —OC(O)—, —C(O)O—, —OSO₂—, —S(O₂)O—, —C(O)S—, —SC(O)—, —C(O)C(O)—, —C(O)N(Rₐ)—, —N(Rₐ)C(O)—, —S(O₂)N(Rₐ)—, —N(Rₐ)S(O₂)—, —OC(O)O—, —OC(O)S—, —OC(O)N(Rₐ)—, —N(Rₐ)C(O)O—, —N(Rₐ)C(O)S—, —N(Rₐ)C(O)N(Rₐ)—, a bivalent alkenyl group, a bivalent alkynyl group, a bivalent cycloalkyl group, a bivalent heterocycloalkyl group, a bivalent aryl group, a bivalent heteroaryl group;
two of R₁ group, taken together with the atoms to which they are attached, may optionally form a cycloalkyl or heterocycloalkyl, in which said cycloalkyl or heterocycloalkyl of R₁, is optionally substituted with one or more R_{d};
two of R₂ group, taken together with the atom(s) to which they are attached, may optionally form a cycloalkyl or heterocycloalkyl, in which said cycloalkyl or heterocycloalkyl of R₂, is optionally substituted with one or more R_{d};
two of R₇ group, taken together with the atoms to which they are attached, may optionally form a cycloalkyl or heterocycloalkyl, in which said cycloalkyl or heterocycloalkyl of R₇, is optionally substituted with one or more R_{d};
two of R₁₀ group, taken together with the atom(s) to which they are attached, may optionally form a cycloalkyl or heterocycloalkyl, in which said cycloalkyl or heterocycloalkyl of R₁₀, is optionally substituted with one or more R_{d};
R₇ and L group, taken together with the atom to which they are attached, may optionally form a cycloalkyl, or heterocycloalkyl, in which said cycloalkyl or heterocycloalkyl of R₇ and L, is optionally substituted with one or more Rₑ;
R_{b} and R, group, taken together with the atom to which they are attached, may optionally form a cycloalkyl, or heterocycloalkyl, in which said cycloalkyl or heterocycloalkyl of R_{b} and R_{c}, is optionally substituted with one or more Rₑ;
two of R_{d} group, taken together with the atom(s) to which they are attached, may optionally form a cycloalkyl, or heterocycloalkyl, in which said cycloalkyl or heterocycloalkyl of R_{d}, is optionally substituted with one or more Rₑ;
two of Rₑ group, taken together with the atom(s) to which they are attached, may optionally form a cycloalkyl or heterocycloalkyl, in which said cycloalkyl or heterocycloalkyl of Rₑ is optionally substituted with one or more groups selected from H, D, alkyl, alkenyl, alkynyl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, aryl, or heteroaryl;
each of k, g, m, n, p and q is, independently, 0, 1, 2, 3, 4, or 5;
s is 0 or 1; and
f is 0 or 1;
(b) extruding the semi-solid mixture; and
(c) cooling the resulting extrudate to provide a solid matrix comprising the polymeric carrier and the surfactant and having the compound or salt thereof dispersed in an essentially non-crystalline form therein.

30. The process of claim 29, wherein the compound is represented by Formula (A-1), (A-2), or (A-3):

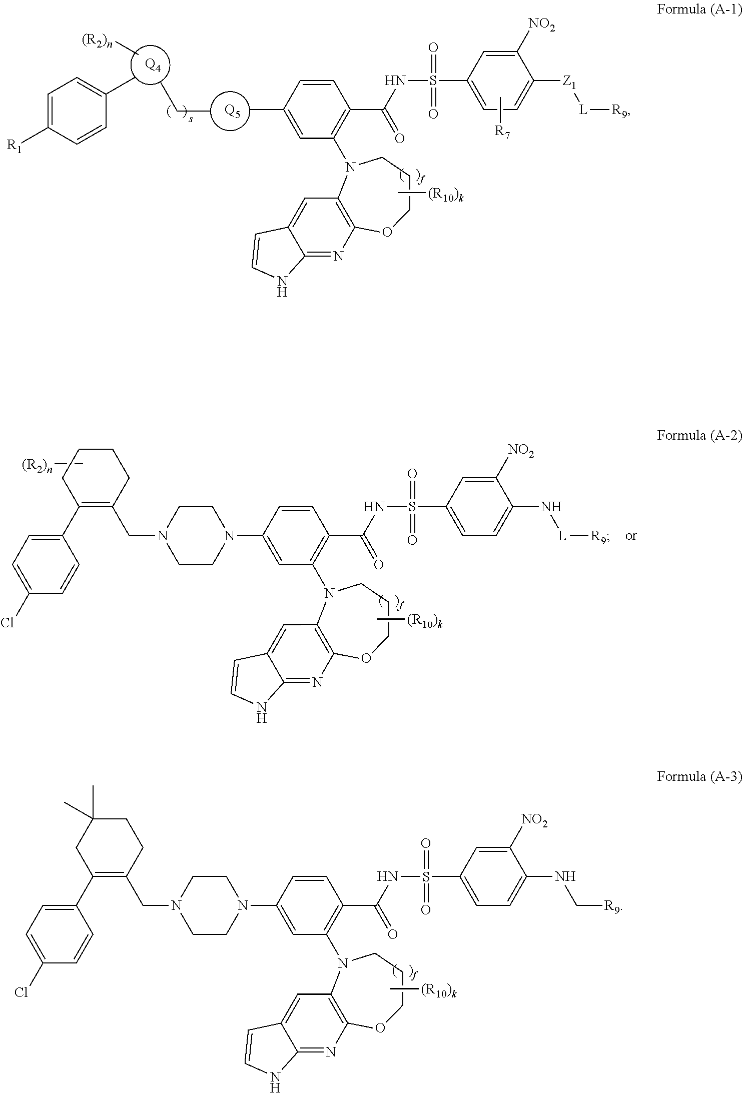

31. (canceled)

32. (canceled)

33. The process of claim 29, wherein
i) the API, polymeric carrier, surfactant, and antioxidant are mixed together before said subjecting to elevated temperature,
ii) the API, polymeric carrier, surfactant, and antioxidant are mixed together while subjecting to elevated temperature:
iii) said elevated temperature is about 100° C. to about 200° C.: or
iv) said elevated temperature is about 125° C. to about 175° C., or about 140-160° C.

34-36. (canceled)

37. The process of claim 33, further comprising calendering the extrudate before or while cooling.

38. The process of claim 37, wherein
i) the polymeric carrier comprises a copovidone, such as a KOLLIDON® VA64 type copovidone;
ii) the surfactant comprises a polysorbate, such as a TWEEN 80 type polysorbate 80: or
iii) the antioxidant comprises ascorbic acid or sodium ascorbate.

39. (canceled)

40. (canceled)

41. An orally deliverable pharmaceutical dosage form comprising the solid dispersion of claim 1.

42. A method for treating a neoplastic, immune or autoimmune disease, comprising orally administering to a subject having the disease a therapeutically effective amount of the solid dispersion of claim 1.

43. The method of claim 42, wherein
i) the disease is a neoplastic disease,
ii) the neoplastic disease is selected from the group consisting of cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, non-Hodgkin's lymphoma, Hodgkin's lymphoma, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma and combinations thereof;
iii) the neoplastic disease is chronic lymphocytic leukemia or acute lymphocytic leukemia;
iv) the neoplastic disease is non-Hodgkin's lymphoma or Hodgkin's lymphoma:
v) the disease is an immune or autoimmune disease:
vi) the solid dispersion is administered in a parent-compound-equivalent dose of about 10 to about 1,000 mg per day of the compound of Formula A or salt thereof at an average treatment interval of about 6 hours to about 7 days: or
vii) the compound is selected from the group consisting of
(S)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(R)-4-(4-((4′-chloro-5,5-dimethyl-3,4,5,6-tetrahydro-[1,1′-biphenyl]-2-yl)methyl)piperazin-1-yl)-2-(3-methyl-2,3-dihydropyrrolo[3′,2′:5,6]pyrido[2,3-b][1,4]oxazin-1(6H)-yl)-N-((3-nitro-4-(((tetrahydro-2H-pyran-4-yl)methyl)amino)phenyl)sulfonyl)benzamide,
(S)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((4-(((4-fluorotetrahydro-2H-pyran-4-yl)methyl)amino)-3-nitrophenyl)sulfonyl)-2-(3-methyl-2,3-dihydropyrrolo[3′,2′:5,6]pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-3-methyl-2,3-dihydropyrrolo[3′,2′:5,61pyrido[2,3-b][1,4]oxazin-1(6H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-3-methyl-2,3-dihydropyrrolo[3′,2′:5,61pyrido[2,3-b][1,4]oxazin-1(6H)-yl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((3-nitro-4-(((tetrahydro-2H-pyran-4-yl)methyl)amino)phenyl)sulfonyl)benzamide,
4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)-N-((4-(((4-fluorotetrahydro-2H-pyran-4-yl)methyl)amino)-3-nitrophenyl)sulfonyl)benzamide,
(S)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-N-((4-(((1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-(3,4-dihydro-2H-pyrrolo[3′,2′:5,6]pyrido[2,3-b][1,4]oxazepin-1(7H)-yl)benzamide,
(R)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
(S)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)-2-(4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((R)-4-(trifluoromethyl)-3,4-dihydro-2H-pyrrolo[3′,2′:5,6]pyrido[2,3-b][1,4]oxazepin-1(7H)-yl)benzamide,
N-((4-((((S)-1,4-dioxan-2-yl)methyl)amino)-3-nitrophenyl)sulfonyl)-4-(4-((4′-chloro-5,5-dimethyl-3,4,5,6-tetrahydro-[1,1′-biphenyl]-2-yl)methyl)piperazin-1-yl)-2-((S)-4-(trifluoromethyl)-3,4-dihydro-2H-pyrrolo[3′,2′:5,6]pyrido[2,3-b][1,4]oxazepin-1(7H)-yl)benzamide.

44-49. (canceled)
